(19) United States
(12) Patent Application Publication
(10) Pub. No.: US 2024/0271116 A1
(43) Pub. Date: Aug. 15, 2024

(54) SUPPRESSION OF PAIN BY GENE EDITING

(71) Applicant: President and Fellows of Harvard College, Cambridge, MA (US)

(72) Inventors: Juan Pablo Maianti, Cambridge, MA (US); David R. Liu, Cambridge, MA (US)

(73) Assignee: President and Fellows of Harvard College, Cambridge, MA (US)

(21) Appl. No.: 18/545,977

(22) Filed: Dec. 19, 2023

Related U.S. Application Data
(63) Continuation of application No. 16/492,548, filed on Sep. 9, 2019, now Pat. No. 11,898,179.
(60) Provisional application No. 62/469,408, filed on Mar. 9, 2017.

(51) Int. Cl. C12N 9/78; A61K 9/00; A61K 31/7088; A61K 38/46; A61K 38/50; A61K 48/00; C12N 9/22; C12N 9/96; C12N 15/11

(52) U.S. Cl. CPC C12N 9/78; A61K 9/0029; A61K 9/0053; A61K 31/7088; A61K 38/465; A61K 38/50; C12N 9/22; C12N 9/96; C12N 15/11; C12Y 305/04; A61K 48/00; C07K 2319/00; C12N 2310/20; C12N 2320/31; C12N 2320/32; C12N 2800/80

(57) ABSTRACT

Provided herein are systems, compositions, kits, and methods for the suppression of pain (e.g., chronic pain). Genes encoding ion channels (e.g., SCN9A) responsible for the propagation pain signals in neurons (e.g., DRG neurons) may be edited using a genome editing agent (e.g., a nucleobase editor). In some embodiments, loss-of-function ion channel mutants are generated, leading to pain suppression. In some embodiments, the genome editing agent is administered locally to the site of pain or to the nerves responsible for propagation of the pain signal.

### RELATED APPLICATIONS

The present application is a national stage filing under 35 U.S.C. § 371 of international PCT application, PCT/US2018/021664, filed Mar. 9, 2018, which claims priority under 35 U.S.C. § 119(e) to U.S. provisional application, U.S. Ser. No. 62/469,408, filed Mar. 9, 2017, each of which is incorporated herein by reference.

### REFERENCE TO A SEQUENCE LISTING SUBMITTED AS A TEXT FILE VIA EFS-WEB

This application contains a Sequence Listing which has been submitted in ASCII format via EFS-Web and is hereby incorporated by reference in its entirety. Said ASCII copy, created on Jul. 17, 2023, is named H082470245US02-SUBSEQ-AZW and is 4,153,363 bytes in size.

### BACKGROUND OF THE INVENTION

Long-term chronic pain due to trauma and associated with advanced cancer as well as other causes remains an unmet medical need. Management of pain using painkillers is inherently limited by the development of tolerance, physiological dependence, progressive addiction, and potential for overdose. Current health care policies in response to the massive demand for painkillers have led to extensive prescription of opioids, inadvertently contributing to broader public challenges associated with substance abuse and drug-related crime. Fundamentally, there is a pressing need for an innovative solution to address chronic pain that is non-addictive, generalizable, and/or permanent.

### SUMMARY OF THE INVENTION

Described herein are systems, compositions, kits, and methods for the suppression of pain (e.g., chronic pain). The strategies rely, at least in part, on the targeted editing of genes encoding proteins (e.g., ion channels such as Nav1.7 encoded by the SCN9A gene) responsible for the propagation of pain signals in sensory neurons that display dysregulated excitability, e.g., in dorsal root ganglia (DRG) neurons. The targeted genome editing may be achieved, in some embodiments, using a genome editing agent, e.g., a nucleobase editor comprising a catalytically inactive Cas9 or a Cas9 nickase and a cytosine deaminase. The nucleobase editor introduces cytosine (C) to thymine (T) mutations in the targeted gene. In some embodiments, loss-of-function ion channel mutants are generated, leading to pain suppression. In some embodiments, the genome editing agent is administered locally to the site of pain. The pain suppression strategies provided herein are effective in long-term pain suppression and have high safety profiles. In some embodiments, neurotropic viral delivery vectors are used to specifically deliver the genome editing agent to neurons. In some embodiments, neuron-specific promoters are used to drive the expression of the genome editing agents specifically in neurons.

Some aspects of the present disclosure provide methods of editing a polynucleotide encoding an ion channel in a dorsal root ganglia (DRG) neuron, the method comprising contacting the ion channel-encoding polynucleotide with: (i) a fusion protein comprising: (a) a guide nucleotide sequence-programmable DNA binding protein domain; and (b) a cytosine deaminase domain; and (ii) a guide nucleotide sequence targeting the fusion protein of (i) to a target cytosine (C) base in the ion channel-encoding polynucleotide, whereby the contacting results in deamination of the target C base by the fusion protein, resulting in a cytosine (C) to thymine (T) change in the ion channel-encoding polynucleotide. In some embodiments, the guide nucleotide sequence-programmable DNA binding protein domain is selected from the group consisting of: nuclease inactive Cas9 (dCas9) domains, nuclease inactive Cpf1 domains, nuclease inactive Argonaute domains, and variants thereof.

In some embodiments, the guide nucleotide sequence-programmable DNA-binding protein domain is a nuclease inactive Cas9 (dCas9) domain. In some embodiments, the dCas9 domain is from Streptococcus pyogenes. In some embodiments, the amino acid sequence of the dCas9 domain includes mutations corresponding to a D10A and/or H840A mutation in SEQ ID NO: 1. In some embodiments, the amino acid sequence of the dCas9 domain includes a mutation corresponding to a D10A mutation in SEQ ID NO: 1, and wherein the dCas9 domain includes a histidine at the position corresponding to amino acid 840 of SEQ ID NO: 1. In some embodiments, the guide nucleotide sequence-programmable DNA-binding protein domain comprises a nuclease inactive Cpf1 (dCpf1) domain. In some embodiments, the dCpf1domain is from a species of Acidaminococcus or Lachnospiraceae. In some embodiments, the guide nucleotide sequence-programmable DNA-binding protein domain comprises a nuclease inactive Argonaute (dAgo) domain. In some embodiments, the dAgo domain is from Natronobacterium gregoryi (dNgAgo).

In some embodiments, the cytosine deaminase domain comprises an apolipoprotein B mRNA-editing complex (APOBEC) family deaminase. In some embodiments, the cytosine deaminase is selected from the group consisting of APOBEC1, APOBEC2, APOBEC3A, APOBEC3B, APOBEC3C, APOBEC3D, APOBEC3F, APOBEC3G deaminase, APOBEC3H deaminase, APOBEC4 deaminase, activation-induced deaminase (AID), and pmCDA1. In some embodiments, the cytosine deaminase comprises the amino acid sequence of any one of SEQ ID NOs: 271-292 and 303.

In some embodiments, the fusion protein further comprises a uracil glycosylase inhibitor (UGI) domain. In some embodiments, the UGI domain comprises the amino acid sequence of SEQ ID NO: 304.

In some embodiments, the cytosine deaminase domain is fused to the N-terminus of the guide nucleotide sequence-programmable DNA-binding protein domain. In some embodiments, the UGI domain is fused to the C-terminus of the guide nucleotide sequence-programmable DNA-binding protein domain.

In some embodiments, the cytosine deaminase and the guide nucleotide sequence-programmable DNA-binding protein domain are fused via an optional linker. In some embodiments, the UGI domain is fused to the dCas9 domain via an optional linker.

In some embodiments, the fusion protein has the structure NH₂-[cytosine deaminase domain]-[optional linker sequence]-[guide nucleotide sequence-programmable DNA-binding protein domain]-[optional linker sequence]-[UGI domain]-COOH. In some embodiments, the fusion protein has the structure NH₂-[UGI domain]-[optional linker sequence]-[cytosine deaminase domain]-[optional linker sequence]-[guide nucleotide sequence-programmable DNA-binding protein domain]-COOH. In some embodiments, the fusion protein has the structure NH₂-[cytosine deaminase domain]-[optional linker sequence]-[guide nucleotide sequence-programmable DNA-binding protein domain]-COOH.

In some embodiments, the linker comprises (GGGS)ₙ (SEQ ID NO: 2430), (GGGGS)ₙ (SEQ ID NO: 308), (G)ₙ (SEQ ID NO: 2498), (EAAAK)ₙ (SEQ ID NO: 309), (GGS)ₙ (SEQ ID NO: 2467), SGSETPGTSESATPES (SEQ ID NO: 310), or (XP)ₙ motif, or a combination of any of these, wherein n is independently an integer between 1 and 30, and wherein X is any amino acid. In some embodiments, the linker comprises the amino acid sequence SGSETPGTSESATPES (SEQ ID NO: 310). In some embodiments, the linker is (GGS)ₙ (SEQ ID NO: 2467), and wherein n is 1, 3, or 7.

In some embodiments, the fusion protein comprises the amino acid sequence of any one of SEQ ID NO: 10, 293-302, and 2495.

In some embodiments, the polynucleotide encoding the ion channel comprises a coding strand and a complementary strand. In some embodiments, the polynucleotide encoding the ion channel comprises a coding region and a non-coding region. In some embodiments, the C to T change occurs in the coding region of the ion channel-encoding polynucleotide. In some embodiments, the C to T change leads to a mutation in the ion channel.

In some embodiments, the mutation introduces a premature stop codon in the ion channel-coding sequence that leads to a truncated or non-functional ion channel. In some embodiments, the premature stop codon is TAG (Amber), TGA (Opal), or TAA (Ochre). In some embodiments, the mutation destabilizes ion-channel protein folding. In some embodiments, the C to T change occurs at a C base-paired with the G base in a start codon (AUG).

In some embodiments, the C to T change occurs at the non-coding region of the ion channel-encoding polynucleotide. In some embodiments, the C to T change occurs at a splicing site in the non-coding region of the ion channel-encoding polynucleotide. In some embodiments, the C to T change occurs at an intron-exon junction. In some embodiments, the C to T change occurs at a splicing donor site. In some embodiments, the C to T change occurs at a splicing acceptor site.

In some embodiments, the ion channel is selected from the group consisting of: NaV1.7, NaV1.8. NaV1.9, NaV1.3, CaV3.2, HCN1, HCN2, and Ano1. In some embodiments, the ion channel is NaV1.7 encoded by the SCN9A gene.

In some embodiments, the mutation is a loss-of-function mutation. In some embodiments, the C to T change occurs in a target codon selected from Tables 2, 4, and 6. In some embodiments, the guide nucleotide sequence is selected from SEQ ID NOs: 339-1456.

In some embodiments, a PAM sequence is located 3′ of the C being changed. In some embodiments, a PAM sequence is located 5′ of the C being changed. In some embodiments, the PAM sequence is selected from the group consisting of: NGG, NGAN, NGNG, NGAG, NGCG, NNGRRT, NGGNG, NGRRN, NNNRRT, NNNGATT, NNAGAA, and NAAAC, wherein Y is pyrimidine, R is purine, and N is any nucleobase. In some embodiments, the PAM sequence is selected from the group consisting of: NNT, NNNT, and YNT, wherein Y is pyrimidine, and N is any nucleobase. In some embodiments, no PAM sequence is located 3′ of the target C base. In some embodiments, no PAM sequence is located 5′ of the target C base.

In some embodiments, at least 1, 2, 3, 4, 5, 6, 7, 8, 9, or 10 mutations are introduced into the ion channel-encoding polynucleotide. In some embodiments, the guide nucleotide sequence is RNA (gRNA). In some embodiments, the guide nucleotide sequence is ssDNA (gDNA).

In some embodiments, the DRG neuron is in a mammal. In some embodiments, the mammal is a rodent. In some embodiments, the rodent is a mouse. In some embodiments, the rodent is a rat. In some embodiments, the mammal is a human.

In some embodiments, a nucleic acid construct encoding the fusion protein is delivered to the DRG neuron via a neurotropic viral delivery vector. In some embodiments, the neurotropic viral delivery vector is derived from Herpesviridae, varicella zoster virus, pseudorabies virus, cyromegalovirus, Epstein-barr virus, encephalitis virus, polio virus, coxsackie virus, echo virus, mumps virus, measles virus, and rabies virus. In some embodiments, the neurotropic viral delivery vector is derived from Herpes Simplex Virus 1 (HSV-1). In some embodiments, the neurotropic viral delivery vector is derived from a recombinant adeno-associated virus (AAV).

Other aspects of the present disclosure provide compositions comprising: (i) a fusion protein comprising: (a) a guide nucleotide sequence-programmable DNA binding protein domain; and (b) a cytosine deaminase domain; and (ii) a guide nucleotide sequence targeting the fusion protein of (i) to a target cytosine (C) base in an ion channel-encoding polynucleotide.

Further provided herein are compositions comprising a neurotropic viral delivery vector comprising a nucleic acid encoding: (i) a fusion protein comprising: (a) a guide nucleotide sequence-programmable DNA binding protein domain; and (b) a cytosine deaminase domain; and (ii) a guide nucleotide sequence targeting the fusion protein of (i) to a target cytosine (C) base in an ion channel-encoding polynucleotide.

In some embodiments, the guide nucleotide sequence comprises the nucleotide sequence of any one of SEQ ID NOs: 339-1456, 1504-2425, and 2443-2445. In some embodiments, the composition further comprises a pharmaceutically acceptable carrier. Kits comprising the compositions described herein are also provided.

Other aspects of the present disclosure provide methods of suppressing pain, the method comprising administering to a subject in need thereof a therapeutically effective amount of the composition described herein.

In some embodiments, the pain is chronic pain. In some embodiments, the pain is selected from the group consisting of: neuropathic pain, allodynia, hyperalgesia, dysesthesia, causalgia, neuralgia, and arthralgia. In some embodiments, the pain is associated with cancer, tumor pressure, bone metastasis, chemotherapy peripheral neuropathy, radiculopathy (sciatica, lumbar, cervical, failed back surgery syndrome), piriformis syndrome, phantom pain, arachnoiditis, fibromyalgia, facet joint mediated pain, sympathetically-mediated pain syndrome such as complex regional pain syndromes (crps), sacroiliac (si) joint mediated pain, meralgia paresthetica, localized myofacial pain syndromes-myofacial trigger points, diffuse myofacial pain syndrome, post-herpetic neuralgia, trigeminal neuralgia, glossopharyngeal neuralgia, scar pain (post-epesiotomy, post-hernia repair, post-surgery, post-radiotherapy), vulvodynia, vaginismus, levator ani syndrome, chronic prostatitis, interstitial cystitis, first bite syndrome, rheumatoid arthritis pain, osteoarthritis pain, atypical odontalgia, phantom tooth pain, neuropathic orofacial pain, primary erythermalgia and atypical facial pain.

In some embodiments, the subject is a mammal. In some embodiments, the mammal is a rodent. In some embodiments, the rodent is a mouse. In some embodiments, the rodent is a rat. In some embodiments, the mammal is a human. In some embodiments, the mammal is a companion animal. In some embodiments, the companion animal is a dog, a cat, a horses, a cattle, a pig, a sheep, a goat, a chicken, a mouse, a rat, a guinea pig, or a hamster. In some embodiments, the composition is administered orally or parenterally.

The details of certain embodiments of the invention are set forth in the Detailed Description of Certain Embodiments, as described below. Other features, objects, and advantages of the invention will be apparent from the Definitions, Examples, Figures, and Claims.

### BRIEF DESCRIPTION OF THE DRAWINGS

The accompanying drawings, which constitute a part of this specification, illustrate several embodiments of the invention and together with the description, serve to explain the principles of the invention.

FIGS. 1A-1C show schematic representations of exemplary ion channels and signal transmission in doral root ganglion (DRG) neurons. FIG. 1A is a schematic representing a DRG neuron extending an axonal projection that expresses specialized ion channels for triggering and propagating action potentials (AP) in response to stimuli. The body of the neuron resides in a ganglion near the spinal cord together with thousands of other neurons. The output from the dendrites of this neuron inside the spinal cord involve the release of the neurotransmitters, such as glutamate and substance P, and become the pain signals propagated by the spinal cord that are interpreted as signals of pain by the brain. Adapted from Reference 1 in the “References” section. FIG. 1B is a schematic representing a programmable genome editing treatment with a localized neurotropic viral vector to deliver an expression construct into the axon, exploiting the retrograde transport mechanisms to the nucleus of a dorsal root ganglion (DRG) neuron to modify one of the specialized genes that mediate the threshold or transmission of action potentials that are interpreted as pain (Table 12). FIG. 1C is a schematic representing the outcome (green arrows) of programmable genome editing treatment with a topologically localized neurotropic viral vector, for example, targeting the NaV1.7/SCN9a gene.

FIG. 2 shows exemplary, non-limiting representative examples of genome modifications using cytidine deaminase base editing, which can be applied to modify DRG neuron genes and afferent pain signals.

FIG. 3 shows a two-dimensional representation of the primary amino acid sequence of an isoform of NaV1.7/SCN9A, highlighting the transmembrane regions. The circles show non-limiting examples of variants that can be generated by genome modifications using cytidine deaminase base editing, which can be applied to modify the NaV1.7/SCN9A gene and afferent pain signals. The NaV1.7/Scn9A ion channel is shown as a non-limiting example of ion channels of DRG neurons. Other possible modifications, such as intron/exon junctions are not shown for clarity (see, e.g., FIG. 4).

FIGS. 4A-4B. FIG. 4A shows non-limiting examples of the results obtained from C-+T base editing treatments using guide-RNAs targeted to the NaV1.7/SCN9A gene in the mouse Neuro-2a cell line, analysed using Illumina MiSeq high-throughput DNA sequencing. The treatments shown generate premature STOP codons or modify intron/exon junctions involved in mRNA splicing. FIG. 4B is a two-dimensional representation of the primary amino acid sequence of an isoform of mouse NaV1.7/SCN9A highlighting the sites targeted in panel A and other representative sites that can be targeted in the same manner (black). Additional possible modifications are not shown for clarity (see, e.g., FIG. 3).

FIGS. 5A-5B show representative plots obtained from C-T base editing treatments targeted to the NaV1.7/SCN9A gene in the mouse Neuro-2a cell line, analysed using Illumina MiSeq high-throughput DNA sequencing. FIG. 5A shows the S. pyogenes Cas9 DNA-binding domain fused to APOBEC and UGI (SpBE4), and FIG. 5B shows the KKH variant of the S. aureus Cas9 DNA-binding domain fused to APOBEC and UGI (KKH-SaBE3). The X axis sequence is underlined at the PAM and the end of the protospacer-targeting region is marked with a horizontal line. A dashed box highlights a target codon that is modified to a premature STOP codon by C to T base-editors acting on either the forward (coding) or reverse (template) strand of genomic DNA. The protospacer sequences in Figure SA correspond from top to bottom to SEQ ID NOs: 2447-2457, with 2457 repeated twice at the end.

FIGS. 6A-6C. FIG. 6A shows representative examples of the results obtained from active wild-type S. pyogenes Cas9 treatments using guide-RNAs targeted to the NaV1.7/SCN9A gene in the mouse Neuro-2a cell line, analysed using Illumina MiSeq high-throughput DNA sequencing. FIG. 6B is a gel electrophoresis analysis of PCR products following wild-type S. pyogenes Cas9 treatment using two or more guide-RNAs targeted to the NaV1.7/SCN9A gene in the mouse Neuro-2a cell line, which generate indels and longer deletions between the predicted target sites. The uncut genomic site (plus small indels) are seen as a high molecular-weight band, and large deletions are seen as the lower molecular-weight bands. FIG. 6C shows a representative analysis by Illumina MiSeq high-throughput DNA showing the large deletion product following wild-type S. pyogenes Cas9 treatment using two guide-RNAs, g3 and g12 from FIG. 6B, targeted to the NaV1.7/SCN9A gene in the mouse Neuro-2a cell line sequencing. The protospacer sequences in FIG. 6A correspond from top to bottom to SEQ ID NOs: 2458-2466 and 2457.

### DEFINITIONS

As used herein and in the claims, the singular forms “a,” “an,” and “the” include the singular and the plural reference unless the context clearly indicates otherwise. Thus, for example, a reference to “an agent” includes a single agent and a plurality of such agents.

A “dorsal root ganglion (DRG),” also referred to as a “spinal ganglion” or “posterior root ganglion,” is a cluster of nerve cell bodies (a ganglion) in the posterior root of a spinal nerve. A neuron in the DRG is referred to herein as a “dorsal root ganglia (DRG) neuron.” The dorsal root ganglia contain the cell bodies of sensory neurons.

A “neuron” is an electrically excitable cell that processes and transmits information through electrical and chemical signals. These signals between neurons occur via synapses, specialized connections with other cells. Neurons can connect to each other to form neural networks. Neurons are the core components of the brain and spinal cord of the central nervous system (CNS), and of the ganglia of the peripheral nervous system (PNS).

There are several types of specialized neurons: sensory neurons, motor neurons, and interneurons. A “sensory neuron” is a neuron that responds to stimuli such as touch, sound, or light, and all other stimuli affecting the cells of the sensory organs that then send signals to the spinal cord and brain. A “motor neuron” is a neuron that receives signals from the brain and spinal cord to cause muscle contractions and affect glandular outputs. A “interneuron” is a neuron that connects neurons to other neurons within the same region of the brain or spinal cord in neural networks.

In the PNS, an afferent nerve fiber is the axon of an afferent sensory neuron. It is a long process extending far from the nerve cell body that carries nerve impulses from sensory receptors or sense organs toward the central nervous system. The opposite direction of neural activity is termed efferent conduction.

Neurons are electrically excitable, maintaining voltage gradients across their membranes by means of metabolically driven ion pumps, which combine with ion channels embedded in the membrane to generate intracellular-versus-extracellular concentration differences of ions, such as sodium, potassium, chloride, and calcium. Changes in the cross-membrane voltage can alter the function of voltage-dependent ion channels. If the voltage changes by a large enough amount, an all-or-none electrochemical pulse called an action potential is generated, which travels rapidly along the cell's axon, and activates synaptic connections with other cells when it arrives.

An “ion channel” is a pore-forming membrane protein expressed on the surface of a cell (e.g., a DRG neuron). Ion channels on the surface of a cell (e.g., a DRG neuron) have various biological functions including: establishing a resting membrane potential, shaping action potentials and other electrical signals by gating the flow of ions across the cell membrane, controlling the flow of ions across secretory and epithelial cells, and regulating cell volume. Activated transmembrane ion channels allow ions into or out of cells. Genes encoding ion channels in DRG neurons that are responsible for propagation of pain are provided in Example 2.

“Hyperalgesia” is an increased sensitivity to pain, which may be caused by damage to nociceptors or peripheral nerves. Temporary increased sensitivity to pain also occurs as part of sickness behavior, the evolved response to infection. Long-term opioid (e.g. heroin, morphine) users and those on high-dose opioid medications for the treatment of chronic pain may experience hyperalgesia and experience pain out of proportion to physical findings, which is a common cause for loss of efficacy of these medications over time.

“Allodynia” refers to central pain sensitization (increased response of neurons) following normally non-painful, often repetitive, stimulation. Allodynia can lead to the triggering of a pain response from stimuli which do not normally provoke pain. Temperature or physical stimuli can provoke allodynia, which may feel like a burning sensation. Allodynia often occurs after injury to a site. Allodynia is different from hyperalgesia, an extreme, exaggerated reaction to a stimulus which is normally painful.

The term “loss-of-function mutation” or “inactivating mutation” refers to a mutation that results in the gene product having less or no function (being partially or wholly inactivated). When the allele has a complete loss of function (null allele), it is often called an amorphic mutation in the Muller's morphs schema. Phenotypes associated with such mutations are most often recessive. Exceptions are when the organism is haploid, or when the reduced dosage of a normal gene product is not enough for a normal phenotype (this is called haploinsufficiency).

The term “gain-of-function mutation” or “activating mutation” refers to a mutation that changes the gene product such that its effect gets stronger (enhanced activation) or even is superseded by a different and abnormal function. A gain of function mutation may also be referred to as a neomorphic mutation. When the new allele is created, a heterozygote containing the newly created allele as well as the original will express the new allele, genetically defining the mutations as dominant phenotypes.

The term “genome” refers to the genetic material of a cell or organism. It typically includes DNA (or RNA in the case of RNA viruses). The genome includes both the genes, the coding regions, the noncoding DNA, and the genomes of the mitochondria and chloroplasts. A genome does not typically include genetic material that is artificially introduced into a cell or organism, e.g., a plasmid that is transformed into a bacteria is not a part of the bacterial genome.

A “programmable DNA-binding protein” refers to DNA binding proteins that can be programmed to target to any desired nucleotide sequence within a genome. To program the DNA-binding protein to bind a desired nucleotide sequence, the DNA binding protein may be modified to change its binding specificity. e.g., zinc finger DNA-binding domain, zinc finger nuclease (ZFN), or transcription activator-like effector proteins (TALE). ZFNs are artificial restriction enzymes generated by fusing a zinc finger DNA-binding domain to a DNA-cleavage domain. Zinc finger domains can be engineered to target specific desired DNA sequences, and this enables zinc-fingers to bind unique sequences within complex genomes. Transcription activator-like effector nucleases (TALEN) are engineered restriction enzymes that can be engineered to cut specific sequences of DNA. They are made by fusing a TAL effector DNA-binding domain to a nuclease domain (e.g. Fok1). Transcription activator-like effectors (TALEs) can be engineered to bind practically any desired DNA sequence. Methods for programming ZFNs and TALEs are familiar to one skilled in the art. For example, such methods are described in Maeder et al., Mol. Cell 31 (2): 294-301, 2008; Carroll et al., Genetics Society of America, 188 (4): 773-782, 2011; Miller et al., Nature Biotechnology 25 (7): 778-785, 2007; Christian et al., Genetics 186 (2): 757-61, 2008; Li et al., Nucleic Acids Res. 39 (1): 359-372, 2010; and Moscou et al., Science 326 (5959): 1501, 2009, each of which are incorporated herein by reference.

A “guide nucleotide sequence-programmable DNA-binding protein” refers to a protein, a polypeptide, or a domain that is able to bind DNA, and the binding to its target DNA sequence is mediated by a guide nucleotide sequence. Thus, it is appreciated that the guide nucleotide sequence-programmable DNA-binding protein binds a guide nucleotide sequence. The “guide nucleotide” may be an RNA or DNA molecule (e.g., a single-stranded DNA or ssDNA molecule) that is complementary to the target sequence and can guide the DNA binding protein to the target sequence. As such, a guide nucleotide sequence-programmable DNA-binding protein may be a RNA-programmable DNA-binding protein (e.g., a Cas9 protein), or an ssDNA-programmable DNA-binding protein (e.g., an Argonaute protein). “Programmable” means the DNA-binding protein may be programmed to bind any DNA sequence that the guide nucleotide targets.

In some embodiments, the guide nucleotide sequence exists as a single nucleotide molecule and comprises two domains: (1) a domain that shares homology to a target nucleic acid (e.g., and directs binding of a guide nucleotide sequence-programmable DNA-binding protein to the target); and (2) a domain that binds a guide nucleotide sequence-programmable DNA-binding protein. In some embodiments, domain (2) corresponds to a sequence known as a tracrRNA and comprises a stem-loop structure. For example, in some embodiments, domain (2) is identical or homologous to a tracrRNA as provided in Jinek et al., Science 337:816-821(2012), which is incorporated herein by reference. Other examples of gRNAs (e.g., those including domain 2) can be found in U.S. Patent Application Publication US 2016/0208288 and U.S. Patent Application Publication US 2016/0200779, each of which is incorporated herein by reference.

Because the guide nucleotide sequence hybridizes to a target DNA sequence, the guide nucleotide sequence-programmable DNA-binding proteins are able to specifically bind, in principle, to any sequence complementary to the guide nucleotide sequence. Methods of using guide nucleotide sequence-programmable DNA-binding protein, such as Cas9, for site-specific editing of the genome (with or without cleaving the double stranded DNA) are known in the art (see e.g., Cong, L. et al. Multiplex genome engineering using CRISPR/Cas systems. Science 339, 819-823 (2013); Mali, P. et al. RNA-guided human genome engineering via Cas9. Science 339, 823-826 (2013); Hwang. W. Y. et al. Efficient genome editing in zebrafish using a CRISPR-Cas system. Nature Biotechnology 31, 227-229 (2013); Jinek, M. et al. RNA-programmed genome editing in human cells. eLife 2, e00471 (2013); Dicarlo, J. E. et al. Genome engineering in Saccharomyces cerevisiae using CRISPR-Cas systems. Nucleic Acids Research (2013); Jiang, W. et al. RNA-guided editing of bacterial genomes using CRISPR-Cas systems. Nature Biotechnology 31, 233-239 (2013); each of which is incorporated herein by reference).

As used herein, the term “Cas9” or “Cas9 nuclease” refers to an RNA-guided nuclease comprising a Cas9 protein, a fragment, or a variant thereof. A Cas9 nuclease is also referred to sometimes as a casn1 nuclease or a CRISPR (clustered regularly interspaced short palindromic repeat)-associated nuclease. CRISPR is an adaptive immune system that provides protection against mobile genetic elements (viruses, transposable elements and conjugative plasmids). CRISPR clusters contain spacers, sequences complementary to antecedent mobile elements, and target invading nucleic acids. CRISPR clusters are transcribed and processed into CRISPR RNA (crRNA). In type II CRISPR systems correct processing of pre-crRNA requires a trans-encoded small RNA (tracrRNA), endogenous ribonuclease 3 (rnc), and a Cas9 protein. The tracrRNA serves as a guide for ribonuclease 3-aided processing of pre-crRNA. Subsequently, Cas9/crRNA/tracrRNA endonucleolytically cleaves a linear or circular dsDNA target complementary to the spacer. The target strand not complementary to the crRNA is first cut endonucleolytically, then trimmed 3′-5′ exonucleolytically. In nature, DNA-binding and cleavage typically requires protein and both RNAs. However, single guide RNAs (“sgRNA”, or simply “gNRA”) can be engineered so as to incorporate aspects of both the crRNA and tracrRNA into a single RNA species. See, e.g., Jinek et al., Science 337:816-821(2012), which is incorporated herein by reference.

Cas9 nuclease sequences and structures are well known to those of skill in the art (see, e.g., Ferretti et al., Proc. Natl. Acad. Sci. 98:4658-4663(2001); Deltcheva E. et al., Nature 471:602-607(2011); and Jinek et al., Science 337:816-821(2012), each of which is incorporated herein by reference). Cas9 orthologs have been described in various species. Additional suitable Cas9 nucleases and sequences will be apparent to those of skill in the art based on this disclosure, and such Cas9 nucleases and sequences include Cas9 sequences from the organisms and loci disclosed in Chylinski et al., (2013) RNA Biology 10:5, 726-737; which are incorporated herein by reference. In some embodiments, wild type Cas9 corresponds to Cas9 from Streptococcus pyogenes (NCBI Reference Sequence: NC_002737.2, SEQ ID NO: 5 (nucleotide); and Uniport Reference Sequence: Q99ZW2, SEQ ID NO: 1 (amino acid).

(SEQ ID NO: 5)
ATGGATAAGAAATACTCAATAGGCTTAGATATCGGCACAAATAGCGTCGGATGGGCGGTGATCAC
TGATGAATATAAGGTTCCGTCTAAAAAGTTCAAGGTTCTGGGAAATACAGACCGCCACAGTATCA
AAAAAAATCTTATAGGGGCTCTTTTATTTGACAGTGGAGAGACAGCGGAAGCGACTCGTCTCAAA
CGGACAGCTCGTAGAAGGTATACACGTCGGAAGAATCGTATTTGTTATCTACAGGAGATTTTTTCA
AATGAGATGGCGAAAGTAGATGATAGTTTCTTTCATCGACTTGAAGAGTCTTTTTTGGTGGAAGAA
GACAAGAAGCATGAACGTCATCCTATTTTTGGAAATATAGTAGATGAAGTTGCTTATCATGAGAAA
TATCCAACTATCTATCATCTGCGAAAAAAATTGGTAGATTCTACTGATAAAGCGGATTTGCGCTTA
ATCTATTTGGCCTTAGCGCATATGATTAAGTTTCGTGGTCATTTTTTGATTGAGGGAGATTTAAATC
CTGATAATAGTGATGTGGACAAACTATTTATCCAGTTGGTACAAACCTACAATCAATTATTTGAAG
AAAACCCTATTAACGCAAGTGGAGTAGATGCTAAAGCGATTCTTTCTGCACGATTGAGTAAATCAA
GACGATTAGAAAATCTCATTGCTCAGCTCCCCGGTGAGAAGAAAAATGGCTTATTTGGGAATCTCA
TTGCTTTGTCATTGGGTTTGACCCCTAATTTTAAATCAAATTTTGATTTGGCAGAAGATGCTAAATT
ACAGCTTTCAAAAGATACTTACGATGATGATTTAGATAATTTATTGGCGCAAATTGGAGATCAATA
TGCTGATTTGTTTTTGGCAGCTAAGAATTTATCAGATGCTATTTTACTTTCAGATATCCTAAGAGTA
AATACTGAAATAACTAAGGCTCCCCTATCAGCTTCAATGATTAAACGCTACGATGAACATCATCAA
GACTTGACTCTTTTAAAAGCTTTAGTTCGACAACAACTTCCAGAAAAGTATAAAGAAATCTTTTTT
GATCAATCAAAAAACGGATATGCAGGTTATATTGATGGGGGAGCTAGCCAAGAAGAATTTTATAA
ATTTATCAAACCAATTTTAGAAAAAATGGATGGTACTGAGGAATTATTGGTGAAACTAAATCGTGA
AGATTTGCTGCGCAAGCAACGGACCTTTGACAACGGCTCTATTCCCCATCAAATTCACTTGGGTGA
GCTGCATGCTATTTTGAGAAGACAAGAAGACTTTTATCCATTTTTAAAAGACAATCGTGAGAAGAT
TGAAAAAATCTTGACTTTTCGAATTCCTTATTATGTTGGTCCATTGGCGCGTGGCAATAGTCGTTTT
GCATGGATGACTCGGAAGTCTGAAGAAACAATTACCCCATGGAATTTTGAAGAAGTTGTCGATAA
AGGTGCTTCAGCTCAATCATTTATTGAACGCATGACAAACTTTGATAAAAATCTTCCAAATGAAAA
AGTACTACCAAAACATAGTTTGCTTTATGAGTATTTTACGGTTTATAACGAATTGACAAAGGTCAA
ATATGTTACTGAAGGAATGCGAAAACCAGCATTTCTTTCAGGTGAACAGAAGAAAGCCATTGTTG
ATTTACTCTTCAAAACAAATCGAAAAGTAACCGTTAAGCAATTAAAAGAAGATTATTTCAAAAAA
ATAGAATGTTTTGATAGTGTTGAAATTTCAGGAGTTGAAGATAGATTTAATGCTTCATTAGGTACC
TACCATGATTTGCTAAAAATTATTAAAGATAAAGATTTTTTGGATAATGAAGAAAATGAAGATATC
TTAGAGGATATTGTTTTAACATTGACCTTATTTGAAGATAGGGAGATGATTGAGGAAAGACTTAAA
ACATATGCTCACCTCTTTGATGATAAGGTGATGAAACAGCTTAAACGTCGCCGTTATACTGGTTGG
GGACGTTTGTCTCGAAAATTGATTAATGGTATTAGGGATAAGCAATCTGGCAAAACAATATTAGAT
TTTTTGAAATCAGATGGTTTTGCCAATCGCAATTTTATGCAGCTGATCCATGATGATAGTTTGACAT
TTAAAGAAGACATTCAAAAAGCACAAGTGTCTGGACAAGGCGATAGTTTACATGAACATATTGCA
AATTTAGCTGGTAGCCCTGCTATTAAAAAAGGTATTTTACAGACTGTAAAAGTTGTTGATGAATTG
GTCAAAGTAATGGGGCGGCATAAGCCAGAAAATATCGTTATTGAAATGGCACGTGAAAATCAGAC
AACTCAAAAGGGCCAGAAAAATTCGCGAGAGCGTATGAAACGAATCGAAGAAGGTATCAAAGAA
TTAGGAAGTCAGATTCTTAAAGAGCATCCTGTTGAAAATACTCAATTGCAAAATGAAAAGCTCTAT
CTCTATTATCTCCAAAATGGAAGAGACATGTATGTGGACCAAGAATTAGATATTAATCGTTTAAGT
GATTATGATGTCGATCACATTGTTCCACAAAGTTTCCTTAAAGACGATTCAATAGACAATAAGGTC
TTAACGCGTTCTGATAAAAATCGTGGTAAATCGGATAACGTTCCAAGTGAAGAAGTAGTCAAAAA
GATGAAAAACTATTGGAGACAACTTCTAAACGCCAAGTTAATCACTCAACGTAAGTTTGATAATTT
AACGAAAGCTGAACGTGGAGGTTTGAGTGAACTTGATAAAGCTGGTTTTATCAAACGCCAATTGG
TTGAAACTCGCCAAATCACTAAGCATGTGGCACAAATTTTGGATAGTCGCATGAATACTAAATACG
ATGAAAATGATAAACTTATTCGAGAGGTTAAAGTGATTACCTTAAAATCTAAATTAGTTTCTGACT
TCCGAAAAGATTTCCAATTCTATAAAGTACGTGAGATTAACAATTACCATCATGCCCATGATGCGT
ATCTAAATGCCGTCGTTGGAACTGCTTTGATTAAGAAATATCCAAAACTTGAATCGGAGTTTGTCT
ATGGTGATTATAAAGTTTATGATGTTCGTAAAATGATTGCTAAGTCTGAGCAAGAAATAGGCAAA
GCAACCGCAAAATATTTCTTTTACTCTAATATCATGAACTTCTTCAAAACAGAAATTACACTTGCA
AATGGAGAGATTCGCAAACGCCCTCTAATCGAAACTAATGGGGAAACTGGAGAAATTGTCTGGGA
TAAAGGGCGAGATTTTGCCACAGTGCGCAAAGTATTGTCCATGCCCCAAGTCAATATTGTCAAGAA
AACAGAAGTACAGACAGGCGGATTCTCCAAGGAGTCAATTTTACCAAAAAGAAATTCGGACAAGC
TTATTGCTCGTAAAAAAGACTGGGATCCAAAAAAATATGGTGGTTTTGATAGTCCAACGGTAGCTT
ATTCAGTCCTAGTGGTTGCTAAGGTGGAAAAAGGGAAATCGAAGAAGTTAAAATCCGTTAAAGAG
TTACTAGGGATCACAATTATGGAAAGAAGTTCCTTTGAAAAAAATCCGATTGACTTTTTAGAAGCT
AAAGGATATAAGGAAGTTAAAAAAGACTTAATCATTAAACTACCTAAATATAGTCTTTTTGAGTTA
GAAAACGGTCGTAAACGGATGCTGGCTAGTGCCGGAGAATTACAAAAAGGAAATGAGCTGGCTCT
GCCAAGCAAATATGTGAATTTTTTATATTTAGCTAGTCATTATGAAAAGTTGAAGGGTAGTCCAGA
AGATAACGAACAAAAACAATTGTTTGTGGAGCAGCATAAGCATTATTTAGATGAGATTATTGAGC
AAATCAGTGAATTTTCTAAGCGTGTTATTTTAGCAGATGCCAATTTAGATAAAGTTCTTAGTGCAT
ATAACAAACATAGAGACAAACCAATACGTGAACAAGCAGAAAATATTATTCATTTATTTACGTTG
ACGAATCTTGGAGCTCCCGCTGCTTTTAAATATTTTGATACAACAATTGATCGTAAACGATATACG
TCTACAAAAGAAGTTTTAGATGCCACTCTTATCCATCAATCCATCACTGGTCTTTATGAAACACGC
ATTGATTTGAGTCAGCTAGGAGGTGACTGA
(SEQ ID NO: 1)
MDKKYSIGLDIGTNSVGWAVITDEYKVPSKKFKVLGNTDRHSIKKNLIGALLFDSGETAEATRLKRTARR
RYTRRKNRICYLQEIFSNEMAKVDDSFFHRLEESFLVEEDKKHERHPIFGNIVDEVAYHEKYPTIYHLR
KKLVDSTDKADLRLIYLALAHMIKFRGHFLIEGDLNPDNSDVDKLFIQLVQTYNQLFEENPINASGVDA
KAILSARLSKSRRLENLIAQLPGEKKNGLFGNLIALSLGLTPNFKSNFDLAEDAKLQLSKDTYDDDLDN
LLAQIGDQYADLFLAAKNLSDAILLSDILRVNTEITKAPLSASMIKRYDEHHQDLTLLKALVRQQLPEK
YKEIFFDQSKNGYAGYIDGGASQLEFYKFIKPILEKMDGTEELLVKLNREDLLRKQRTFDNGSIPHQIH
LGELHAILRRQEDFYPFLKDNREKIEKILTFRIPYYVGPLARGNSRFAWMTRKSEETITPWNFEEVVDK
GASAQSFIERMTNFDKNLPNEKVLPKHSLLYEYFTVYNELTKVKYVTEGMRKPAFLSGEQKKAIVDLLF
KTNRKVTVKQLKEDYFKKIECFDSVEISGVEDRFNASLGTYHDLLKIIKDKDFLDNEENEDILEDIVLT
LTLFEDREMIEERLKTYAHLFDDKVMKQLKRRRYTGWGRLSRKLINGIRDKQSGKTILDFLKSDGFANR
NFMQLIHDDSLTFKEDIQKAQVSGQGDSLHEHIANLAGSPAIKKGILQTVKVVDELVKVMGRHKPENIVI
EMARENQTTQKGQKNSRERMKRIEEGIKELGSQILKEHPVENTQLQNEKLYLYYLQNGRDMYVDQEL
DINRLSDYDVDHIVPQSFLKDDSIDNKVLTRSDKNRGKSDNVPSEEVVKKMKNYWRQLLNAKLITQRK
FDNLTKAERGGLSELDKAGFIKRQLVETRQITKHVAQILDSRMNTKYDENDKLIREVKVITLKSKLVSD
FRKDFQFYKVREINNYHHAHDAYLNAVVGTALIKKYPKLESEFVYGDYKVYDVRKMIAKSEQEIGKA
TAKYFFYSNIMNFFKTEITLANGEIRKRPLIETNGETGEIVWDKGRDFATVRKVLSMPQVNIVKKTEVQ
TGGFSKESILPKRNSDKLIARKKDWDPKKYGGFDSPTVAYSVLVVAKVEKGKSKKLKSVKELLGITIME
RSSFEKNPIDFLEAKGYKEVKKDLIIKLPKYSLFELENGRKRMLASAGELQKGNELALPSKYVNFLYLA
SHYEKLKGSPEDNEQKQLFVEQHKHYLDEIIEQISEFSKRVILADANLDKVLSAYNKHRDKPIREQAEN
IIHLFTLTNLGAPAAFKYFDTTIDRKRYTSTKEVLDATLIHQSITGLYETRIDLSQLGGD
(single underline: HNH domain; double underline: RuvC domain)

In some embodiments, wild type Cas9 corresponds to Cas9 from Streptococcus Aureus. S. aureus Cas9 wild type (SEQ ID NO: 6)

MKRNYILGLDIGITSVGYGIIDYETRDVIDAGVRLFKEANVENNE
GRRSKRGARRLKRRRRHRIQRVKKLLFDYNLLTDHSELSGINPYE
ARVKGLSQKLSEEEFSAALLHLAKRRGVHNVNEVEEDTGNELSTK
EQISRNSKALEEKYVAELQLERLKKDGEVRGSINRFKTSDYVKEA
KQLLKVQKAYHQLDQSFIDTYIDLLETRRTYYEGPGEGSPFGWKD
IKEWYEMLMGHCTYFPEELRSVKYAYNADLYNALNDLNNLVITRD
ENEKLEYYEKFQIIENVFKQKKKPTLKQIAKEILVNEEDIKGYRV
TSTGKPEFTNLKVYHDIKDITARKEIIENAELLDQIAKILTIYQS
SEDIQEELTNLNSELTQLEIEQISNLKGYTGTHNLSLKAINLILD
ELWHTNDNQIAIFNRLKLVPKKVDLSQQKEIPTTLVDDFILSPVV
KRSFIQSIKVINAIIKKYGLPNDIIIELAREKNSKDAQKMINEMQ
KRNRQTNERIEEIIRTTGKENAKYLIEKIKLHDMQEGKCLYSLEA
IPLEDLLNNPFNYEVDHIIPRSVSFDNSFNNKVLVKQEENSKKGN
RTPFQYLSSSDSKISYETFKKHILNLAKGKGRISKTKKEYLLEER
DINRFSVQKDFINRNLVDTRYATRGLMNLLRSYFRVNNLDVKVKS
INGGFTSFLRRKWKFKKERNKGYKHHAEDALIIANADFIFKEWKK
LDKAKKVMENQMFEEKQAESMPEIETEQEYKEIFITPHQIKHIKD
FKDYKYSHRVDKKPNRELINDTLYSTRKDDKGNTLIVNNLNGLYD
KDNDKLKKLINKSPEKLLMYHHDPQTYQKLKLIMEQYGDEKNPLY
KYYEETGNYLTKYSKKDNGPVIKKIKYYGNKLNAHLDITDDYPNS
RNKVVKLSLKPYRFDVYLDNGVYKFVTVKNLDVIKKENYYLVNSK
CYLEAKKLKKISNQAEFIASFYNNDLIKINGELYRVIGVNNDLLN
RIEVNMIDITYREYLENMNDKRPPRIIKTIASKTQSIKKYSTDIL
GNLYEVKSKKHPQIIKKG

In some embodiments, wild type Cas9 corresponds to Cas9 from Streptococcus thermophilus.

Streptococcus thermophilus wild type
CRISPR3 Cas9 (St3Cas9)
(SEQ ID NO: 7)
MTKPYSIGLDIGTNSVGWAVITDNYKVPSKKMKVLGNTSKKYIKKN
LLGVLLFDSGITAEGRRLKRTARRRYTRRRNRILYLQEIFSTEMAT
LDDAFFQRLDDSFLVPDDKRDSKYPIFGNLVEEKVYHDEFPTIYHL
RKYLADSTKKADLRLVYLALAHMIKYRGHFLIEGEFNSKNNDIQKN
FQDFLDTYNAIFESDLSLENSKQLEEIVKDKISKLEKKDRILKLFP
GEKNSGIFSEFLKLIVGNQADFRKCFNLDEKASLHFSKESYDEDLE
TLLGYIGDDYSDVFLKAKKLYDAILLSGFLTVTDNETEAPLSSAMI
KRYNEHKEDLALLKEYIRNISLKTYNEVFKDDTKNGYAGYIDGKTN
QEDFYVYLKNLLAEFEGADYFLEKIDREDFLRKQRTFDNGSIPYQI
HLQEMRAILDKQAKFYPFLAKNKERIEKILTFRIPYYVGPLARGNS
DFAWSIRKRNEKITPWNFEDVIDKESSAEAFINRMTSFDLYLPEEK
VLPKHSLLYETFNVYNELTKVRFIAESMRDYQFLDSKQKKDIVRLY
FKDKRKVTDKDIIEYLHAIYGYDGIELKGIEKQFNSSLSTYHDLLN
IINDKEFLDDSSNEAIIEEIIHTLTIFEDREMIKQRLSKFENIFDK
SVLKKLSRRHYTGWGKLSAKLINGIRDEKSGNTILDYLIDDGISNR
NFMQLIHDDALSFKKKIQKAQIIGDEDKGNIKEVVKSLPGSPAIKK
GILQSIKIVDELVKVMGGRKPESIVVEMARENQYTNQGKSNSQQRL
KRLEKSLKELGSKILKENIPAKLSKIDNNALQNDRLYLYYLQNGKD
MYTGDDLDIDRLSNYDIDHIIPQAFLKDNSIDNKVLVSSASNRGKS
DDFPSLEVVKKRKTFWYQLLKSKLISQRKFDNLTKAERGGLLPEDK
AGFIQRQLVETRQITKHVARLLDEKFNNKKDENNRAVRTVKIITLK
STLVSQFRKDFELYKVREINDFHHAHDAYLNAVIASALLKKYPKLE
PEFVYGDYPKYNSFRERKSATEKVYFYSNIMNIFKKSISLADGRVI
ERPLIEVNEETGESVWNKESDLATVRRVLSYPQVNVVKKVEEQNHG
LDRGKPKGLFNANLSSKPKPNSNENLVGAKEYLDPKKYGGYAGISN
SFAVLVKGTIEKGAKKKITNVLEFQGISILDRINYRKDKLNFLLEK
GYKDIELIIELPKYSLFELSDGSRRMLASILSTNNKRGEIHKGNQI
FLSQKFVKLLYHAKRISNTINENHRKYVENHKKEFEELFYYILEFN
ENYVGAKKNGKLLNSAFQSWQNHSIDELCSSFIGPTGSERKGLFEL
TSRGSAADFEFLGVKIPRYRDYTPSSLLKDATLIHQSVTGLYETRI
DLAKLGEG
Streptococcus thermophilus CRISPR1 Cas9
wild type (St1Cas9)
(SEQ ID NO: 8)
MSDLVLGLDIGIGSVGVGILNKVTGEIIHKNSRIFPAAQAENNLV
RRTNRQGRRLTRRKKHRRVRLNRLFEESGLITDFTKISINLNPYQ
LRVKGLTDELSNEELFIALKNMVKHRGISYLDDASDDGNSSIGDY
AQIVKENSKQLETKTPGQIQLERYQTYGQLRGDFTVEKDGKKHRL
INVFPTSAYRSEALRILQTQQEFNPQITDEFINRYLEILTGKRKY
YHGPGNEKSRTDYGRYRTSGETLDNIFGILIGKCTFYPDEFRAAK
ASYTAQEFNLLNDLNNLTVPTETKKLSKEQKNQIINYVKNEKAMG
PAKLFKYIAKLLSCDVADIKGYRIDKSGKAEIHTFEAYRKMKTLE
TLDIEQMDRETLDKLAYVLTLNTEREGIQEALEHEFADGSFSQKQ
VDELVQFRKANSSIFGKGWHNFSVKLMMELIPELYETSEEQMTIL
TRLGKQKTTSSSNKTKYIDEKLLTEEIYNPVVAKSVRQAIKIVNA
AIKEYGDFDNIVIEMARETNEDDEKKAIQKIQKANKDEKDAAMLK
AANQYNGKAELPHSVFHGHKQLATKIRLWHQQGERCLYTGKTISI
HDLINNSNQFEVDHILPLSITFDDSLANKVLVYATANQEKGQRTP
YQALDSMDDAWSFRELKAFVRESKTLSNKKKEYLLTEEDISKFDV
RKKFIERNLVDTRYASRVVLNALQEHFRAHKIDTKVSVVRGQFTS
QLRRHWGIEKTRDTYHHHAVDALIIAASSQLNLWKKQKNTLVSYS
EDQLLDIETGELISDDEYKESVFKAPYQHFVDTLKSKEFEDSILF
SYQVDSKFNRKISDATIYATRQAKVGKDKADETYVLGKIKDIYTQ
DGYDAFMKIYKKDKSKFLMYRHDPQTFEKVIEPILENYPNKQINE
KGKEVPCNPFLKYKEEHGYIRKYSKKGNGPEIKSLKYYDSKLGNH
IDITPKDSNNKVVLQSVSPWRADVYFNKTTGKYEILGLKYADLQF
EKGTGTYKISQEKYNDIKKKEGVDSDSEFKFTLYKNDLLLVKDTE
TKEQQLFRFLSRTMPKQKHYVELKPYDKQKFEGGEALIKVLGNVA
NSGQCKKGLGKSNISIYKVRTDVLGNQHIIKNEGDKPKLDF

In some embodiments, the Cas9 domain of any of the fusion proteins provided herein is a Cas9 from archaea (e.g. nanoarchaea), which constitute a domain and kingdom of single-celled prokaryotic microbes. In some embodiments, the Cas9 domain is CasX or CasY, which have been described in, for example, Burstein et al., “New CRISPR-Cas systems from uncultivated microbes.” Cell Res. 2017 Feb. 21. doi: 10.1038/cr.2017.21, which is incorporated herein by reference. Using genome-resolved metagenomics, a number of CRISPR-Cas systems were identified, including the first reported Cas9 in the archaeal domain of life. This divergent Cas9 protein was found in nanoarchaea as part of an active CRISPR-Cas system. In bacteria, two previously unknown systems were discovered, CRISPR-CasX and CRISPR-CasY, which are among the most compact systems yet discovered. In some embodiments, Cas9 refers to CasX, or a variant of CasX. In some embodiments, Cas9 refers to a CasY, or a variant of CasY. It should be appreciated that other RNA-guided DNA binding proteins may be used as a nucleic acid programmable DNA binding protein (napDNAbp) and are within the scope of this disclosure.

In some embodiments, the Cas9 domain comprises an amino acid sequence that is at least 85%, at least 90%, at least 91%, at least 92%, at least 93%, at least 94%, at least 95%, at least 96%, at least 97%, at least 98%, at least 99%, or at least 99.5% identical to a naturally-occurring CasX or CasY protein. In some embodiments, the Cas9 domain is a naturally-occurring CasX or CasY protein. In some embodiments, the Cas9 domain comprises an amino acid sequence that is at least 85%, at least 90%, at least 91%, at least 92%, at least 93%, at least 94%, at least 95%, at least 96%, at least 97%, at least 98%, at least 99%, or at least 99.5% identical to any one of SEQ ID NOs: 336-337. In some embodiments, the Cas9 domain comprises an amino acid sequence of any one SEQ ID NOs: 336-337. It should be appreciated that CasX and CasY from other bacterial species may also be used in accordance with the present disclosure.

In some embodiments, wild-type Cas9 refers to CasX from Sulfolobus islandicus (strain REY15A).

(SEQ ID NO: 336)
MEVPLYNIFGDNYIIQVATEAENSTIYNNKVEIDDEELRNVLNLAY
KIAKNNEDAAAERRGKAKKKKGLEGETTTSNIILPLSGNDKNPWTE
TLKCYNFPTTVALSEVFKNFSQVKECEEVSAPSFVKPEFYKFGRSP
GMVERTRRVKLEVEPHYLIMAAAGWVLTRLGKAKVSEGDYVGVNVF
TPTRGILYSLIQNVNGIVPGIKPETAFGLWIARKVVSSVTNPNVSV
VSIYTISDAVGQNPTTINGGFSIDLTKLLEKRDLLSERLEAIARNA
LSISSNMRERYIVLANYIYEYLTGSKRLEDLLYFANRDLIMNLNSD
DGKVRDLKLISAYVNGELIRGEG

In some embodiments, wild-type Cas9 refers to CasX from Sulfolobus islandicus (strain REY15A).

(SEQ ID NO: 337)
MEVPLYNIFGDNYIIQVATEAENSTIYNNKVEIDDEELRNVLNLAY
KIAKNNEDAAAERRGKAKKKKGLEGETTTSNIILPLSGNDKNPWTE
TLKCYNFPTTVALSEVFKNFSQVKECEEVSAPSFVKPEFYEFGRSP
GMVERTRRVKLEVEPHYLIIAAAGWVLTRLGKAKVSEGDYVGVNVF
TPTRGILYSLIQNVNGIVPGIKPETAFGLWIARKVVSSVTNPNVSV
VRIYTISDAVGQNPTTINGGFSIDLTKLLEKRYLLSERLEAIARNA
LSISSNMRERYIVLANYIYEYLTGSKRLEDLLYFANRDLIMNLNSD
DGKVRDLKLISAYVNGELIRGEG

In some embodiments, wild-type Cas9 refers to CasY from a Parcubacteria group bacterium.
CasY (ncbi.nlm.nih.gov/protein/APG80656.1)

CasY (ncbi.nlm.nih.gov/protein/APG80656.1)
>APG80656.1 CRISPR-associated protein CasY
[uncultured Parcubacteria group bacterium]
(SEQ ID NO: 2469)
MSKRHPRISGVKGYRLHAQRLEYTGKSGAMRTIKYPLYSSPSGGRTV
PREIVSAINDDYVGLYGLSNFDDLYNAEKRNEEKVYSVLDFWYDCVQ
YGAVFSYTAPGLLKNVAEVRGGSYELTKTLKGSHLYDELQIDKVIKF
LNKKEISRANGSLDKLKKDIIDCFKAEYRERHKDQCNKLADDIKNAK
KDAGASLGERQKKLFRDFFGISEQSENDKPSFTNPLNLTCCLLPFDT
VNNNRNRGEVLFNKLKEYAQKLDKNEGSLEMWEYIGIGNSGTAFSNF
LGEGFLGRLRENKITELKKAMMDITDAWRGQEQEEELEKRLRILAAL
TIKLREPKFDNHWGGYRSDINGKLSSWLQNYINQTVKIKEDLKGHKK
DLKKAKEMINRFGESDTKEEAVVSSLLESIEKIVPDDSADDEKPDIP
AIAIYRRFLSDGRLTLNRFVQREDVQEALIKERLEAEKKKKPKKRKK
KSDAEDEKETIDFKELFPHLAKPLKLVPNFYGDSKRELYKKYKNAAI
YTDALWKAVEKIYKSAFSSSLKNSFFDTDFDKDFFIKRLQKIFSVYR
RFNTDKWKPIVKNSFAPYCDIVSLAENEVLYKPKQSRSRKSAAIDKN
RVRLPSTENIAKAGIALARELSVAGFDWKDLLKKEEHEEYIDLIELH
KTALALLLAVTETQLDISALDFVENGTVKDFMKTRDGNLVLEGRFLE
MFSQSIVFSELRGLAGLMSRKEFITRSAIQTMNGKQAELLYIPHEFQ
SAKITTPKEMSRAFLDLAPAEFATSLEPESLSEKSLLKLKQMRYYPH
YFGYELTRTGQGIDGGVAENALRLEKSPVKKREIKCKQYKTLGRGQN
KIVLYVRSSYYQTQFLEWFLHRPKNVQTDVAVSGSFLIDEKKVKTRW
NYDALTVALEPVSGSERVFVSQPFTIFPEKSAELEGQRYLGIDIGEY
GIAYTALEITGDSAKILDQNFISDPQLKTLREEVKGLKLDQRRGTFA
MPSTKIARIRESLVHSLRNRIHHLALKHKAKIVYELEVSRFEEGKQK
IKKVYATLKKADVYSEIDADKNLQTTVWGKLAVASEISASYTSQFCG
ACKKLWRAEMQVDETITTQELIGTVRVIKGGTLIDAIKDFMRPPIFD
ENDTPFPKYRDFCDKHHISKKMRGNSCLFICPFCRANADADIQASQT
IALLRYVKEEKKVEDYFERFRKLKNIKVLGQMKKI

In some embodiments, Cas9 refers to Cas9 from: Corynebacterium ulcerans (NCBI Refs: NC_015683.1, NC_017317.1); Corynebacterium diphtheria (NCBI Refs: NC_016782.1, NC_016786.1); Spiroplasma syrphidicola (NCBI Ref: NC_021284.1); Prevotella intermedia (NCBI Ref: NC_017861.1); Spiroplasma taiwanense (NCBI Ref: NC_021846.1); Streptococcus iniae (NCBI Ref: NC_021314.1); Belliella baltica (NCBI Ref: NC_018010.1); Psychroflexus torquisI (NCBI Ref: NC_018721.1); Listeria innocua (NCBI Ref: NP_472073.1); Campylobacter jejuni (NCBI Ref: YP_002344900.1); or Neisseria. meningitidis (NCBI Ref: YP_002342100.1) or to a Cas9 from any of the organisms listed in Example 1 (SEQ ID NOs: 11-260).

To be used as in the fusion protein of the present disclosure as the guide nucleotide sequence-programmable DNA binding protein domain, a Cas9 protein needs to be nuclease inactive. A nuclease-inactive Cas9 protein may interchangeably be referred to as a “dCas9” protein (for nuclease-“dead” Cas9). Methods for generating a Cas9 protein (or a fragment thereof) having an inactive DNA cleavage domain are known (See, e.g., Jinek et al., Science. 337:816-821(2012); Qi et al., (2013) Cell. 28; 152(5):1173-83, each of which are incorporated herein by reference). For example, the DNA cleavage domain of Cas9 is known to include two subdomains, the HNH nuclease subdomain and the RuvCI subdomain. The HNH subdomain cleaves the strand complementary to the gRNA, whereas the RuvCI subdomain cleaves the non-complementary strand. Mutations within these subdomains can silence the nuclease activity of Cas9. For example, the mutations D10A and H840A completely inactivate the nuclease activity of S. pyogenes Cas9 (Jinek et al., Science. 337:816-821(2012); Qi et al., Cell. 28; 152(5):1173-83 (2013)).

dCas9 (D10A and H840A)
(SEQ ID NO: 2)
MDKKYSIGLAIGTNSVGWAVITDEYKVPSKKFKVLGNTDRHSIKKNLIGALLFDSGETAEATRLKRTAR
RRYTRRKNRICYLQEIFSNEMAKVDDSFFHRLEESFLVEEDKKHERHPIFGNIVDEVAYHEKYPTIYHLR
KKLVDSTDKADLRLIYLALAHMIKFRGHFLIEGDLNPDNSDVDKLFIQLVQTYNQLFEENPINASGVDA
KAILSARLSKSRRLENLIAQLPGEKKNGLFGNLIALSLGLTPNFKSNFDLAEDAKLQLSKDTYDDDLDN
LLAQIGDQYADLFLAAKNLSDAILLSDILRVNTEITKAPLSASMIKRYDEHHQDLTLLKALVRQQLPEK
YKEIFFDQSKNGYAGYIDGGASQEEFYKFIKPILEKMDGTEELLVKLNREDLLRKQRTFDNGSIPHQIHL
GELHAILRRQEDFYPFLKDNREKIEKILTFRIPYYVGPLARGNSRFAWMTRKSEETITPWNFEEVVDKGA
SAQSFIERMTNFDKNLPNEKVLPKHSLLYEYFTVYNELTKVKYVTEGMRKPAFLSGEQKKAIVDLLFK
TNRKVTVKQLKEDYFKKIECFDSVEISGVEDRFNASLGTYHDLLKIIKDKDFLDNEENEDILEDIVLTLT
LFEDREMIEERLKTYAHLFDDKVMKQLKRRRYTGWGRLSRKLINGIRDKQSGKTILDFLKSDGFANRN
FMQLIHDDSLTFKEDIQKAQVSGQGDSLHEHIANLAGSPAIKKGILQTVKVVDELVKVMGRHKPENIVI
EMARENQTTQKGQKNSRERMKRIEEGIKELGSQILKEHPVENTQLQNEKLYLYYLQNGRDMYVDQEL
DINRLSDYDVDAIVPQSFLKDDSIDNKVLTRSDKNRGKSDNVPSEEVVKKMKNYWRQLLNAKLITQRK
FDNLTKAERGGLSELDKAGFIKRQLVETRQITKHVAQILDSRMNTKYDENDKLIREVKVITLKSKLVSD
FRKDFQFYKVREINNYHHAHDAYLNAVVGTALIKKYPKLESEFVYGDYKVYDVRKMIAKSEQEIGKA
TAKYFFYSNIMNFFKTEITLANGEIRKRPLIETNGETGEIVWDKGRDFATVRKVLSMPQVNIVKKTEVQ
TGGFSKESILPKRNSDKLIARKKDWDPKKYGGFDSPTVAYSVLVVAKVEKGKSKKLKSVKELLGITIME
RSSFEKNPIDFLEAKGYKEVKKDLIIKLPKYSLFELENGRKRMLASAGELQKGNELALPSKYVNFLYLA
SHYEKLKGSPEDNEQKQLFVEQHKHYLDEIIEQISEFSKRVILADANLDKVLSAYNKHRDKPIREQAENI
IHLFTLTNLGAPAAFKYFDTTIDRKRYTSTKEVLDATLIHQSITGLYETRIDLSQLGGD
(single underline: HNH domain; double underline: RuvC domain)

The dCas9 of the present disclosure encompasses completely inactive Cas9 or partially inactive Cas9. For example, the dCas9 may have one of the two nuclease domain inactivated, while the other nuclease domain remains active. Such a partially active Cas9 may also be referred to as a Cas9 nickase, due to its ability to cleave one strand of the targeted DNA sequence. The Cas9 nickase suitable for use in accordance with the present disclosure has an active HNH domain and an inactive RuvC domain and is able to cleave only the strand of the target DNA that is bound by the sgRNA (which is the opposite strand of the strand that is being edited via cytidine deamination). The Cas9 nickase of the present disclosure may comprise mutations that inactivate the RuvC domain, e.g., a D10A mutation. It is to be understood that any mutation that inactivates the RuvC domain may be included in a Cas9 nickase, e.g., insertion, deletion, or single or multiple amino acid substitution in the RuvC domain. In a Cas9 nickase useful in the present disclosure, while the RuvC domain is inactivated, the HNH domain remains activate. Thus, while the Cas9 nickase may comprise mutations other than those that inactivate the RuvC domain (e.g., D10A), those mutations do not affect the activity of the HNH domain. In a non-limiting Cas9 nickase example, the histidine at position 840 remains unchanged. The sequence of an exemplary Cas9 nickase suitable for the present disclosure is provided below.

S. pyogenes Cas9 Nickase (D10A)
(SEQ ID NO: 3)
MDKKYSIGLAIGTNSVGWAVITDEYKVPSKKFKVLGNTDRHSIKKNLIGALLFDSGETAEATRLKRTAR
RRYTRRKNRICYLQEIFSNEMAKVDDSFFHRLEESFLVEEDKKHERHPIFGNIVDEVAYHEKYPTIYHLR
KKLVDSTDKADLRLIYLALAHMIKFRGHFLIEGDLNPDNSDVDKLFIQLVQTYNQLFEENPINASGVDA
KAILSARLSKSRRLENLIAQLPGEKKNGLFGNLIALSLGLTPNFKSNFDLAEDAKLQLSKDTYDDDLDN
LLAQIGDQYADLFLAAKNLSDAILLSDILRVNTEITKAPLSASMIKRYDEHHQDLTLLKALVRQQLPEKY
KEIFFDQSKNGYAGYIDGGASQEEFYKFIKPILEKMDGTEELLVKLNREDLLRKQRTFDNGSIPHQIHL
GELHAILRRQEDFYPFLKDNREKIEKILTFRIPYYVGPLARGNSRFAWMTRKSEETITPWNFEEVVDKGA
SAQSFIERMTNFDKNLPNEKVLPKHSLLYEYFTVYNELTKVKYVTEGMRKPAFLSGEQKKAIVDLLFKT
NRKVTVKQLKEDYFKKIECFDSVEISGVEDRFNASLGTYHDLLKIIKDKDFLDNEENEDILEDIVLTLT
LFEDREMIEERLKTYAHLFDDKVMKQLKRRRYTGWGRLSRKLINGIRDKQSGKTILDFLKSDGFANRN
FMQLIHDDSLTFKEDIQKAQVSGQGDSLHEHIANLAGSPAIKKGILQTVKVVDELVKVMGRHKPENIVI
EMARENQTTQKGQKNSRERMKRIEEGIKELGSQILKEHPVENTQLQNEKLYLYYLQNGRDMYVDQEL
DINRLSDYDVDHIVPQSFLKDDSIDNKVLTRSDKNRGKSDNVPSEEVVKKMKNYWRQLLNAKLITQRK
FDNLTKAERGGLSELDKAGFIKRQLVETRQITKHVAQILDSRMNTKYDENDKLIREVKVITLKSKLVSD
FRKDFQFYKVREINNYHHAHDAYLNAVVGTALIKKYPKLESEFVYGDYKVYDVRKMIAKSEQEIGKA
TAKYFFYSNIMNFFKTEITLANGEIRKRPLIETNGETGEIVWDKGRDFATVRKVLSMPQVNIVKKTEVQ
TGGFSKESILPKRNSDKLIARKKDWDPKKYGGFDSPTVAYSVLVVAKVEKGKSKKLKSVKELLGITIM
ERSSFEKNPIDFLEAKGYKEVKKDLIIKLPKYSLFELENGRKRMLASAGELQKGNELALPSKYVNFLY
LASHYEKLKGSPEDNEQKQLFVEQHKHYLDEIIEQISEFSKRVILADANLDKVLSAYNKHRDKPIREQ
AENIIHLFTLTNLGAPAAFKYFDTTIDRKRYTSTKEVLDATLIHQSITGLYETRIDLSQLGGD
(single underline: HNH domain; double underline: RuvC domain)
S. aureus Cas9 Nickase (D10A)
(SEQ ID NO: 4)
MKRNYILGLAIGITSVGYGIIDYETRDVIDAGVRLFKEANVENNEGRRSKRGARRLKRRRRHRIQRVKK
LLFDYNLLTDHSELSGINPYEARVKGLSQKLSEEEFSAALLHLAKRRGVHNVNEVEEDTGNELSTKEQI
SRNSKALEEKYVAELQLERLKKDGEVRGSINRFKTSDYVKEAKQLLKVQKAYHQLDQSFIDTYIDLLE
TRRTYYEGPGEGSPFGWKDIKEWYEMLMGHCTYFPEELRSVKYAYNADLYNALNDLNNLVITRDENEKL
EYYEKFQIIENVFKQKKKPTLKQIAKEILVNEEDIKGYRVTSTGKPEFTNLKVYHDIKDITARKEIIEN
AELLDQIAKILTIYQSSEDIQEELTNLNSELTQLEIEQISNLKGYTGTHNLSLKAINLILDELWHTNDNQ
IAIFNRLKLVPKKVDLSQQKEIPTTLVDDFILSPVVKRSFIQSIKVINAIIKKYGLPNDIIIELAREKNS
KDAQKMINEMQKRNRQTNERIEEIIRTTGKENAKYLIEKIKLHDMQEGKCLYSLEAIPLEDLLNNPFNYL
VDHIIPRSVSFDNSFNNKVLVKQEENSKKGNRTPFQYLSSSDSKISYETFKKHILNLAKGKGRISKTKKE
YLLEERDINRFSVQKDFINRNLVDTRYATRGLMNLLRSYFRVNNLDVKVKSINGGFTSFLRRKWKFKKER
NKGYKHHAEDALIIANADFIFKEWKKLDKAKKVMENQMFEEKQAESMPEIETEQEYKEIFITPHQIKHI
KDFKDYKYSHRVDKKPNRELINDTLYSTRKDDKGNTLIVNNLNGLYDKDNDKLKKLINKSPEKLLMYHH
DPQTYQKLKLIMEQYGDEKNPLYKYYEETGNYLTKYSKKDNGPVIKKIKYYGNKLNAHLDITDDYPNSR
NKVVKLSLKPYRFDVYLDNGVYKFVTVKNLDVIKKENYYLVNSKCYLEAKKLKKISNQAEFIASFYNND
LIKINGELYRVIGVNNDLLNRIEVNMIDITYREYLENMNDKRPPRIIKTIASKTQSIKKYSTDILGNLY
EVKSKKHPQIIKKG

It is appreciated that when the term “dCas9” or “nuclease-inactive Cas9” is used herein, it refers to Cas9 variants that are inactive in both HNH and RuvC domains as well as Cas9 nickases. For example, the dCas9 may include the amino acid sequence set forth in SEQ ID NO: 2 or SEQ ID NO: 3. In some embodiments, the dCas9 may comprise other mutations that inactivate RuvC or HNH domain. Additional suitable mutations that inactivate Cas9 will be apparent to those of skill in the art based on this disclosure and knowledge in the field, and are within the scope of this disclosure. Such additional exemplary suitable nuclease-inactive Cas9 domains include, but are not limited to, D839A and/or N863A (See, e.g., Prashant et al., Nature Biotechnology. 2013; 31(9): 833-838, which are incorporated herein by reference), or K603R (See, e.g., Chavez et al., Nature Methods 12, 326-328, 2015, which is incorporated herein by reference). The term Cas9, dCas9, or Cas9 variant also encompasses Cas9, dCas9, or Cas9 variants from any organism. Also appreciated is that dCas9. Cas9 nickase, or other appropriate Cas9 variants from any organisms may be used in accordance with the present disclosure.

A “deaminase” refers to an enzyme that catalyzes the removal of an amine group from a molecule, or deamination, for example through hydrolysis. In some embodiments, the deaminase is a cytidine deaminase, catalyzing the deamination of cytidine (C) to uridine (U), deoxycytidine (dC) to deoxyuridine (dU), or 5-methyl-cytidine to thymidine (T, 5-methyl-U), respectively. Subsequent DNA repair mechanisms ensure that a dU is replaced by T, as described in Komor et al (Nature, Programmable editing of a target base in genomic DNA without double-stranded DNA cleavage, 533, 420-424 (2016), which is incorporated herein by reference). In some embodiments, the deaminase is a cytosine deaminase, catalyzing and promoting the conversion of cytosine to uracil (e.g., in RNA) or thymine (e.g., in DNA). In some embodiments, the deaminase is a naturally-occurring deaminase from an organism, such as a human, chimpanzee, gorilla, monkey, cow, dog, rat, or mouse. In some embodiments, the deaminase is a variant of a naturally-occurring deaminase from an organism, and the variants do not occur in nature. For example, in some embodiments, the deaminase or deaminase domain is at least 50%, at least 55%, at least 60%, at least 65%, at least 70%, at least 75% at least 80%, at least 85%, at least 90%, at least 95%, at least 96%, at least 97%, at least 98%, at least 99%, or at least 99.5% identical to a naturally-occurring deaminase from an organism.

A “cytosine deaminase” refers to an enzyme that catalyzes the chemical reaction “cytosine+H₂O→uracil+NH₃” or “5-methyl-cytosine+H₂O→thymine+NH₃.” As it may be apparent from the reaction formula, such chemical reactions result in a C to U/T nucleobase change. In the context of a gene, such nucleotide change, or mutation, may in turn lead to an amino acid change in the protein, which may affect the protein's function, e.g., loss-of-function or gain-of-function. Subsequent DNA repair mechanisms ensure that uracil bases in DNA are replaced by T, as described in Komor et al (Nature, Programmable editing of a target base in genomic DNA without double-stranded DNA cleavage, 533, 420-424 (2016), which is incorporated herein by reference).

One exemplary suitable class of cytosine deaminases is the apolipoprotein B mRNA-editing complex (APOBEC) family of cytosine deaminases encompassing eleven proteins that serve to initiate mutagenesis in a controlled and beneficial manner. The apolipoprotein B editing complex 3 (APOBEC3) enzyme provides protection to human cells against a certain HIV-1 strain via the deamination of cytosines in reverse-transcribed viral ssDNA. These cytosine deaminases all require a Zn²⁺-coordinating motif (His-X-Glu-X₂₃₋₂₆-Pro-Cys-X₂₋₄-Cys; SEQ ID NO: 1996) and bound water molecule for catalytic activity. The glutamic acid residue acts to activate the water molecule to a zinc hydroxide for nucleophilic attack in the deamination reaction. Each family member preferentially deaminates at its own particular “hotspot,” for example, WRC (W is A or T, R is A or G) for hAID, or TTC for hAPOBEC3F. A recent crystal structure of the catalytic domain of APOBEC3G revealed a secondary structure comprising a five-stranded β-sheet core flanked by six α-helices, which is believed to be conserved across the entire family. The active center loops have been shown to be responsible for both ssDNA binding and in determining “hotspot” identity. Overexpression of these enzymes has been linked to genomic instability and cancer, thus highlighting the importance of sequence-specific targeting. Another suitable cytosine deaminase is the activation-induced cytidine deaminase (AID), which is responsible for the maturation of antibodies by converting cytosines in ssDNA to uracils in a transcription-dependent, strand-biased fashion.

The term “base editors” or “nucleobase editors,” as used herein, broadly refer to any of the fusion proteins described herein. In some embodiments, the nucleobase editors are capable of precisely deaminating a target base to convert it to a different base, e.g., the base editor may target C bases in a nucleic acid sequence and convert the C to T base. For example, in some embodiments, the base editor may be a cytosine deaminase-dCas9 fusion protein. In some embodiments, the base editor may be a cytosine deaminase-Cas9 nickase fusion protein. In some embodiments, the base editor may be a deaminase-dCas9-UGI fusion protein. In some embodiments, the base editor may be an UGI-deaminase-dCas9 fusion protein. In some embodiments, the base editor may be an UGI-deaminase-Cas9 nickase fusion protein. In some embodiments, the base editor may be an APOBEC1-dCas9-UGI fusion protein. In some embodiments, the base editor may be an APOBEC1-Cas9 nickase-UGI fusion protein. In some embodiments, the base editor may be an APOBEC1-dCpf1-UGI fusion protein. In some embodiments, the base editor may be an APOBEC1-dNgAgo-UGI fusion protein. In some embodiments, the base editor may comprise a second UGI domain. Non-limiting exemplary sequences of the nucleobase editors useful in the present disclosure are provided in Example 1, SEQ ID NOs: 293-302 and 2495. Such nucleobase editors and methods of using them for genome editing have been described in the art, e.g., in U.S. Pat. No. 9,068,179, US Patent Application Publications US 2015/0166980, US 2015/0166981, US 2015/0166982, US20150166984, and US20150165054, and US Provisional Applications, U.S. Ser. No. 62/245,828, filed Oct. 23, 2015; 62/279,346, filed Jan. 15, 2016; 62/311,763, filed Mar. 22, 2016; 62/322,178, filed Apr. 13, 2016, 62/357,352, filed Jun. 30, 2016, 62/370,700, filed Aug. 3, 2016; 62/398,490, filed Sep. 22, 2016; 62/408,686, filed Oct. 14, 2016; PCT Application PCT/US2016/058344, filed Oct. 22, 2016; U.S. patent application Ser. No. 15/311,852, filed Oct. 22, 2016; Komor et al. (2017) Improved Base Excision Repair Inhibition and Bateriophage Mu Gam Protein Yields C:G-to-T:A base editors with higher efficiency and product purity. Sci Adv, 3: eaao4774; and in Komor et al., Nature, Programmable editing of a target base in genomic DNA without double-stranded DNA cleavage, 533, 420-424 (2016), the entire contents of each of which is incorporated herein by reference.

The term “target site” or “target sequence” refers to a sequence within a nucleic acid molecule (e.g., a DNA molecule) that is deaminated by the fusion protein provided herein. In some embodiments, the target sequence is a polynucleotide (e.g., a DNA), wherein the polynucleotide comprises a coding strand and a complementary strand. The meaning of a “coding strand” and “complementary strand.” as used herein, is the same as the common meaning of the terms in the art. In some embodiments, the target sequence is a sequence in the genome of a mammal. In some embodiments, the target sequence is a sequence in the genome of a human. In some embodiments, the target sequence is a sequence in the genome of a non-human animal The term “target codon” refers to the amino acid codon that is edited by the base editor and converted to a different codon via deamination. The term “target base” refers to the nucleotide base that is edited by the base editor and converted to a different base via deamination. In some embodiments, the target codon in the coding strand is edited (e.g., deaminated). In some embodiments, the target codon in the complimentary strand is edited (e.g., deaminated).

The term “linker,” as used herein, refers to a chemical group or a molecule linking two molecules or moieties, e.g., two domains of a fusion protein, such as, for example, a nuclease-inactive Cas9 domain and a nucleic acid editing domain (e.g., a deaminase domain). Typically, the linker is positioned between, or flanked by, two groups, molecules, domains, or other moieties and connected to each one via a covalent bond, thus connecting the two. In some embodiments, the linker is an amino acid or a plurality of amino acids (e.g., a peptide or protein). In some embodiments, the linker is an organic molecule, group, polymer (e.g. a non-natural polymer, non-peptidic polymer), or chemical moiety. In some embodiments, the linker is 2-100 amino acids in length, for example, 2, 3, 4, 5, 6, 7, 8, 9, 10, 11, 12, 13, 14, 15, 16, 17, 18, 19, 20, 21, 22, 23, 24, 25, 26, 27, 28, 29, 30, 30-35, 35-40, 40-45, 45-50, 50-60, 60-70, 70-80, 80-90, 90-100, 100-150, or 150-200 amino acids in length. Longer or shorter linkers are also contemplated.

The term “mutation.” as used herein, refers to a substitution of a residue within a sequence, e.g., a nucleic acid or amino acid sequence, with another residue, or a deletion or insertion of one or more residues within a sequence. Mutations are typically described herein by identifying the original residue followed by the position of the residue within the sequence and by the identity of the newly substituted residue. Various methods for making the amino acid substitutions (mutations) provided herein are well known in the art, and are provided by, for example, Green and Sambrook. Molecular Cloning: A Laboratory Manual (4^{th} ed., Cold Spring Harbor Laboratory Press, Cold Spring Harbor. N.Y. (2012)).

The terms “nucleic acid,” and “polynucleotide,” as used herein, refer to a compound comprising a nucleobase and an acidic moiety, e.g., a nucleoside, a nucleotide, or a polymer of nucleotides. Typically, polymeric nucleic acids, e.g., nucleic acid molecules comprising three or more nucleotides are linear molecules, in which adjacent nucleotides are linked to each other via a phosphodiester linkage. In some embodiments, “nucleic acid” refers to individual nucleic acid residues (e.g. nucleotides and/or nucleosides). In some embodiments, “nucleic acid” refers to an oligonucleotide chain comprising three or more individual nucleotide residues. As used herein, the terms “oligonucleotide” and “polynucleotide” can be used interchangeably to refer to a polymer of nucleotides (e.g., a string of at least three nucleotides). In some embodiments, “nucleic acid” encompasses RNA as well as single and/or double-stranded DNA. Nucleic acids may be naturally occurring, for example, in the context of a genome, a transcript, an mRNA, tRNA, rRNA, siRNA, snRNA, a plasmid, cosmid, chromosome, chromatid, or other naturally occurring nucleic acid molecule. On the other hand, a nucleic acid molecule may be a non-naturally occurring molecule, e.g., a recombinant DNA or RNA, an artificial chromosome, an engineered genome, or fragment thereof, or a synthetic DNA, RNA, DNA/RNA hybrid, or including non-naturally occurring nucleotides or nucleosides. Furthermore, the terms “nucleic acid,” “DNA,” “RNA,” and/or similar terms include nucleic acid analogs, e.g., analogs having other than a phosphodiester backbone. Nucleic acids can be purified from natural sources, produced using recombinant expression systems and optionally purified, chemically synthesized, etc. Where appropriate, e.g., in the case of chemically synthesized molecules, nucleic acids can comprise nucleoside analogs such as analogs having chemically modified bases or sugars, and backbone modifications. A nucleic acid sequence is presented in the 5′ to 3′ direction unless otherwise indicated. In some embodiments, a nucleic acid is or comprises natural nucleosides (e.g. adenosine, thymidine, guanosine, cytidine, uridine, deoxyadenosine, deoxythymidine, deoxyguanosine, and deoxycytidine); nucleoside analogs (e.g., 2-aminoadenosine, 2-thiothymidine, inosine, pyrrolo-pyrimidine, 3-methyl adenosine, 5-methylcytidine, 2-aminoadenosine, C5-bromouridine, C5-fluorouridine, C5-iodouridine, C5-propynyl-uridine, C5-propynyl-cytidine, C5-methyleytidine, 2-aminoadenosine, 7-deazaadenosine, 7-deazaguanosine, 8-oxoadenosine, 8-oxoguanosine, 0(6)-methylguanine, and 2-thiocytidine); chemically modified bases; biologically modified bases (e.g., methylated bases); intercalated bases; modified sugars (e.g., 2′-fluororibose, ribose, 2′-deoxyribose, arabinose, and hexose); and/or modified phosphate groups (e.g., phosphorothioates and 5′-N-phosphoramidite linkages).

The terms “protein.” “peptide.” and “polypeptide” are used interchangeably herein, and refer to a polymer of amino acid residues linked together by peptide (amide) bonds. The terms refer to a protein, peptide, or polypeptide of any size, structure, or function. Typically, a protein, peptide, or polypeptide will be at least three amino acids long. A protein, peptide, or polypeptide may refer to an individual protein or a collection of proteins. One or more of the amino acids in a protein, peptide, or polypeptide may be modified, for example, by the addition of a chemical entity such as a carbohydrate group, a hydroxyl group, a phosphate group, a farnesyl group, an isofarnesyl group, a fatty acid group, a linker for conjugation, functionalization, or other modification, etc. A protein, peptide, or polypeptide may also be a single molecule or may be a multi-molecular complex. A protein, peptide, or polypeptide may be just a fragment of a naturally occurring protein or peptide. A protein, peptide, or polypeptide may be naturally occurring, recombinant, or synthetic, or any combination thereof. The term “fusion protein” as used herein refers to a hybrid polypeptide which comprises protein domains from at least two different proteins. One protein may be located at the amino-terminal (N-terminal) portion of the fusion protein or at the carboxy-terminal (C-terminal) protein thus forming an “amino-terminal fusion protein” or a “carboxy-terminal fusion protein,” respectively. A protein may comprise different domains, for example, a nucleic acid binding domain (e.g., the gRNA binding domain of Cas9 that directs the binding of the protein to a target site) and a nucleic acid cleavage domain or a catalytic domain of a nucleic-acid editing protein. In some embodiments, a protein is in a complex with, or is in association with, a nucleic acid, e.g., RNA. Any of the proteins provided herein may be produced by any method known in the art. For example, the proteins provided herein may be produced via recombinant protein expression and purification, which is especially suited for fusion proteins comprising a peptide linker. Methods for recombinant protein expression and purification are well known, and include those described by Green and Sambrook, Molecular Cloning: A Laboratory Manual (4^{th} ed., Cold Spring Harbor Laboratory Press, Cold Spring Harbor, N.Y. (2012)), which are incorporated herein by reference.

The term “subject,” as used herein, refers to an individual organism, for example, an individual mammal. A “subject in need thereof”, refers to an individual who has a disease, a symptom of the disease, or a predisposition toward the disease, with the purpose to cure, heal, alleviate, relieve, alter, remedy, ameliorate, improve, or affect the disease, the symptom of the disease, or the predisposition toward the disease. In some embodiments, the subject is a mammal. In some embodiments, the subject is a non-human primate. In some embodiments, the subject is human. In some embodiments, the mammal is a rodent. In some embodiments, the rodent is a mouse. In some embodiments, the rodent is a rat. In some embodiments, the mammal is a companion animal. A “companion animal” refers to pets and other domestic animals. Non-limiting examples of companion animals include dogs and cats; livestock such as horses, cattle, pigs, sheep, goats, and chickens; and other animals such as mice, rats, guinea pigs, and hamsters.

The term “recombinant” as used herein in the context of proteins or nucleic acids refers to proteins or nucleic acids that do not occur in nature, but are the product of human engineering. For example, in some embodiments, a recombinant protein or nucleic acid molecule comprises an amino acid or nucleotide sequence that comprises at least one, at least two, at least three, at least four, at least five, at least six, or at least seven mutations as compared to any naturally occurring sequence. The fusion proteins (e.g., base editors) useful in the present disclosure are made recombinantly. Recombinant technology is familiar to those skilled in the art.

An “intron” refers to any nucleotide sequence within a gene that is removed by RNA splicing during maturation of the final RNA product. The term intron refers to both the DNA sequence within a gene and the corresponding sequence in RNA transcripts. Sequences that are joined together in the final mature RNA after RNA splicing are exons. Introns are found in the genes of most organisms and many viruses, and can be located in a wide range of genes, including those that generate proteins, ribosomal RNA (rRNA), and transfer RNA (tRNA). When proteins are generated from intron-containing genes. RNA splicing takes place as part of the RNA processing pathway that follows transcription and precedes translation.

An “exon” refers to any part of a gene that will become a part of the final mature RNA produced by that gene after introns have been removed by RNA splicing. The term exon refers to both the DNA sequence within a gene and to the corresponding sequence in RNA transcripts. In RNA splicing, introns are removed and exons are covalently joined to one another as part of generating the mature messenger RNA.

“Splicing” refers to the processing of a newly synthesized messenger RNA transcript (also referred to as a primary mRNA transcript). After splicing, introns are removed and exons are joined together (ligated) for form mature mRNA molecule containing a complete open reading frame that is decoded and translated into a protein. For nuclear-encoded genes, splicing takes place within the nucleus either co-transcriptionally or immediately after transcription. The molecular mechanism of RNA splicing has been extensively described, e.g., in Pagani et al., Nature Reviews Genetics 5, 389-396, 2004; Clancy et al., Nature Education 1 (1): 31, 2011; Cheng et al., Molecular Genetics and Genomics 286 (5-6): 395-410, 2014; Taggart et al., Nature Structural & Molecular Biology 19 (7): 719-2, 2012, the contents of each of which are incorporated herein by reference. One skilled in the art is familiar with the mechanism of RNA splicing.

“Alternative splicing” refers to a regulated process during gene expression that results in a single gene coding for multiple proteins. In this process, particular exons of a gene may be included within or excluded from the final, processed messenger RNA (mRNA) produced from that gene. Consequently, the proteins translated from alternatively spliced mRNAs will contain differences in their amino acid sequence and, often, in their biological functions. Notably, alternative splicing allows the human genome to direct the synthesis of many more proteins than would be expected from its 20,000 protein-coding genes. Alternative splicing is sometimes also termed differential splicing. Alternative splicing occurs as a normal phenomenon in eukaryotes, where it greatly increases the biodiversity of proteins that can be encoded by the genome; in humans, ˜95% of multi-exonic genes are alternatively spliced. There are numerous modes of alternative splicing observed, of which the most common is exon skipping. In this mode, a particular exon may be included in mRNAs under some conditions or in particular tissues, and omitted from the mRNA in others. Abnormal variations in splicing are also implicated in disease; a large proportion of human genetic disorders result from splicing variants. Abnormal splicing variants are also thought to contribute to the development of cancer, and splicing factor genes are frequently mutated in different types of cancer. The regulation of alternative splicing is also described in the art, e.g., in Douglas et al., Annual Review of Biochemistry 72 (1): 291-336, 2003; Pan et al., Nature Genetics 40 (12): 1413-1415, 2008; Martin et al., Nature Reviews 6 (5): 386-398, 2005; Skotheim et al., The international journal of biochemistry & cell biology 39 (7-8): 1432-49, 2007, each of which is incorporated herein by reference.

A “coding frame” or “open reading frame” refers to a stretch of codons that encodes a polypeptide. Since DNA is interpreted in groups of three nucleotides (codons), a DNA strand has three distinct reading frames. The double helix of a DNA molecule has two anti-parallel strands so, with the two strands having three reading frames each, there are six possible frame translations. A functional protein may be produced when translation proceeds in the correct coding frame. An insertion or a deletion of one or two bases in the open reading frame causes a shift in the coding frame that is also referred to as a “frameshift mutation.” A frameshift mutation typical results in premature translation termination and/or truncated or non-functional protein.

A “neurotropic virus” is a virus that is capable of accessing or entering the nervous system and neurovirulent if it is capable of causing disease within the nervous system (e.g., CNS or PNS). Important neuroinvasive viruses include poliovirus, which is highly neurovirulent but weakly neuroinvasive, and rabies virus, which is highly neurovirulent but requires tissue trauma (often resulting from an animal bite) to become neuroinvasive. Neurotropic viral delivery vectors may be derived from neurotropic virus to facilitate the delivery of agents (e.g., therapeutic agents for neurological diseases) to neurons. Non-limiting, exemplary neurotropic viruses that may be used to develop neurotropic viral delivery vectors include: Japanese encephalitis virus, Venezuelan equine encephalitis virus, California encephalitis viruses; polio virus, coxsackie virus, echo virus, mumps virus, measles virus, influenza virus, rabies virus, herpes simplex virus, varicella-zoster virus, Epstein-Barr virus, cytomegalo virus, and HHV-6 virus. Methods of using neurotropic viral delivery vectors to delivery therapeutic agents to neurons have been described in the art, e.g., in Lim et al., Pharmacol Res. 2010 January; 61(1): 14-26; Berges et al., Molecular Therapy, Volume 15, Issue 1, January 2007, Pages 20-29; and Beverly et al., Nature Reviews Neuroscience 4, 353-364, 2003, each of which in incorporated herein by reference.

Other viruses that are known to be suitable for gene transfer may also be used to deliver agents to neurons, e.g., adeno-associated virus (AAV), lentivirus, and retrovirus. An AAV-based neurotropic viral delivery system has recently been described in Deverman et al., Nature Biotechnology 34, 204-209 (2016), incorporated herein by reference. Delivery of a split Cas9 using AAV has also been described, e.g., in Truong et al., Nucl. Acids Res. 43, 6450 (2016), and U.S. Provisional Application 62/408,575, filed Oct. 14, 2016, each of which is incorporated herein by reference.

These and other exemplary substituents are described in more detail in the Detailed Description, Examples, and Claims. The invention is not intended to be limited in any manner by the above exemplary listing of substituents.

### DETAILED DESCRIPTION OF CERTAIN EMBODIMENTS

A normal physiological outcome of trauma, inflammation, and/or nerve injury is the induction of gene expression changes in neighboring nociceptive neurons during the period required for healing. Such changes in gene expression, for example, may facilitate the firing of action potentials by neurons at a lower activation threshold and in turn underlie the sensations of pain (e.g., hyperalgesia (increased pain sensitivity) and allodynia (pain following a normally innocuous stimulus)). Chronic pain develops when the enhanced sensitization of sensory neurons becomes irreversibly established and becomes a persistent maladaptive condition. The functional specialization of sensory neurons is driven by the expression of dedicated ion channel genes (e.g., the ion channel genes listed in Table 12) that fine-tune the membrane polarization to trigger and propagate action potentials in response to stimuli. Accordingly, the etiology of chronic pain can be attributed to, at least in part, the dysregulated expression of one or more genes in one or more neurons.

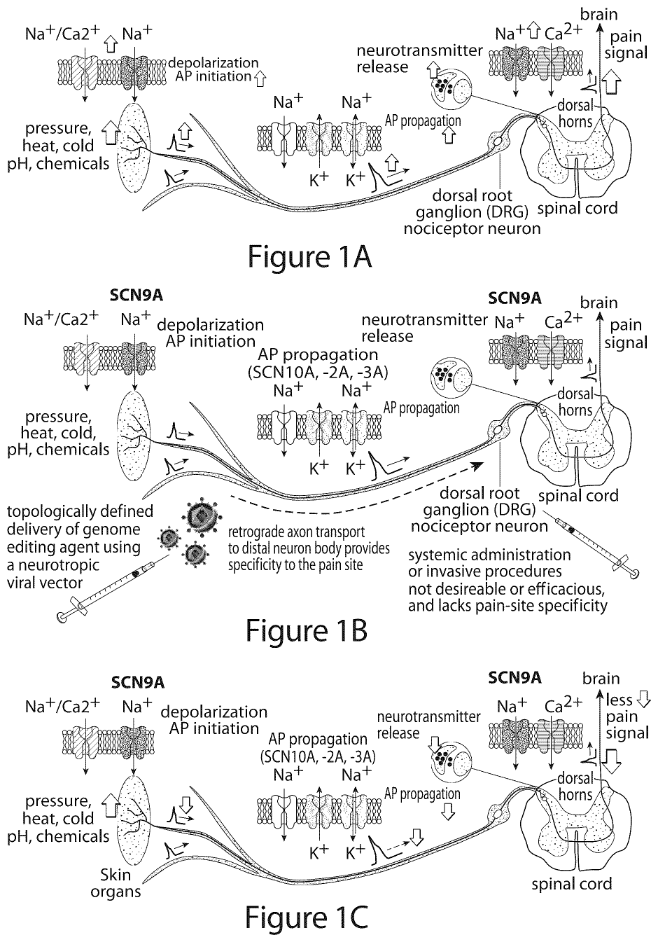

In general, the types of chronic pain that occur in most parts of the body and the extremities involve afferent neurons of the dorsal root ganglia (DRG), which reside in clusters of nerve cells near the spinal cord and have long axons extending towards, for example, the skin, muscles, and organs (FIG. 1). The mechanism of enhanced excitability involves voltage-gated ion channels and background/leak channels that set the resting membrane potential and firing threshold of DRG neurons. Under normal conditions, chemical, mechanical, or thermal stimuli are required to activate receptors and ion channels in peripheral nerve endings to initiate action potentials that propagate along the axons of DRG neurons. In some instances, the dendritic termini of the DRG neurons liberate glutamate and substance-P at synapses in the spinal cord dorsal horn, activating second-order neurons that communicate pain signals to the brain.

Human DRG neurons constitutively express specific and specialized ion channels that have been implicated in afferent pain signaling, which may be targeted for modulation of chronic pain conditions. Three sodium channels (NaV1.7, NaV1.8, and NaV1.9) are constitutively expressed in DRG neurons, and a fourth gene (NaV1.3) displays elevated expression after nerve injury (Table 12). In some embodiments, targeting the ion channels using the strategies described herein leads to gene ablation, loss-of-function, destabilization of the transcript and/or protein folding of the targeted ion channels, which in turn leads to reduced pain transmission. In some embodiments, the normal function of the DRG neurons in triggering action potentials and reaching a normal membrane depolarization threshold is not comprised post editing.

Thus, in some embodiments, a polynucleotide encoding any one of NaV1.7. NaV1.8, NaV1.9, NaV1.3, CaV3.2, HCN1, HCN2, or Ano1 ion-channels is targeted by a genome editing agent (e.g., a nucleobase editor, nuclease). In some embodiments, a polynucleotide (e.g., DNA) encoding NaV1.7 ion channel is targeted.

In a human genome, the NaV1.7 ion channel is encoded by the SCN9A gene. Thus, in some embodiments, the nucleobase editor targets the SCN9A gene in a genome, e.g., a human genome. Disruption of SCN9A is only desirable at a localized level, because nociception is essentially a protective mechanism from overextension and deformation of our joints and muscles, and it is also necessary for our sense of smell. Humans presenting homozygous SCN9A loss-of-function mutations may suffer from congenital insensitivity to pain (CIP). Conversely, gain-of-function mutations in the sodium channels NaV1.7 (SCN9A) or NaV1.8 (SCN10A) cause congenital pain syndromes, such as primary erythermalgia. In some embodiments, the SCN9A gene is involved in itching.

Various genome-editing agents useful in the present disclosure may be deployed to the DRG neurons (e.g., dysregulated DRG neurons to modify the genes responsible for propagation of pain signals in DRG neurons. The strategies for pain (e.g., chronic pain) suppression described herein are superior to traditional methods of pain management due to their high specificity, efficacy, and safety profile. In some embodiments, one or more design elements may be utilized in the strategies described herein that achieves precise and selective targeting of pain-causative neurons. Such design elements include, for example: 1) localized delivery of a non-replicative viral vector that requires synaptic terminals, sparing the bulk of somatic tissues near the pain site, 2) neuron-specific promoters that drive expression of the genome editing construct; and/or 3) guide-RNA programmed targeting of non-essential ion channel genes exclusively expressed by DRG neurons to spare other types of neurons (efferent neurons, interneurons, etc.).

Some aspects of the present disclosure relate to editing a polynucleotide encoding an ion channel in a DRG neuron, the method comprising contacting the ion channel-encoding polynucleotide with a nucleobase editor described herein and a guide nucleotide sequence targeting the nucleobase editor to a target site in the ion channel-encoding polynucleotide. The nucleobase editors described herein target C bases. Contacting the nucleobase editor with a target C base (e.g., a target C base in a ion channel-encoding polynucleotide) results in a cytosine (C) to thymine (T) change in the ion channel-encoding polynucleotide. Such C to T base change ultimately leads to a C:G to T:A base pair change.

### Strategies for Targeting Ion Channels in DRG Neurons

The targeted editing of polynucleotides encoding ion channels in neurons (e.g., DRG neurons) may be achieved, in some embodiments, using nucleobase editors as described in, e.g., U.S. Pat. No. 9,068,179, issued Jun. 30, 2015, US Patent Application Publications US 2015/0166980, US 2015/0166981. US 2015/0166982, US 2015/0166984, and US 2015/0165054, and US Provisional Applications, U.S. Ser. No. 62/245,828, filed Oct. 23, 2015; 62/279,346, filed Jan. 15, 2016; 62/311,763, filed Mar. 22, 2016; 62/322,178, filed Apr. 13, 2016, 62/357,352, filed Jun. 30, 2016, 62/370,700, filed Aug. 3, 2016; 62/398,490, filed Sep. 22, 2016; and 62/408,686, filed Oct. 14, 2016; PCT Application PCT/US2016/058344, filed Oct. 22, 2016; U.S. patent application Ser. No. 15/311,852, filed Oct. 22, 2016; and in Komor et al., Nature, Programmable editing of a target base in genomic DNA without double-stranded DNA cleavage, 533, 420-424 (2016), the entire contents of each of which are incorporated herein by reference.

The nucleobase editors can precisely edit a target base in an ion channel-encoding polynucleotide without introducing a DNA double stand break, thus reducing genome instability and preventing possible oncogenic modifications that may be caused by other genome editing methods. The nucleobase editors described herein may be programmed to target and modify a single base. In some embodiments, the target base is a cytosine (C) base and may be converted to a thymine (T) base via deamination by the nucleobase editor.

In some embodiments, the ion channel-encoding polynucleotide is a DNA molecule comprising a coding strand and a complementary strand, e.g., a gene locus for the ion channel in a genome. The target base may be on either the coding-strand or the complementary strand of an ion channel-encoding polynucleotide. In some embodiments, the ion channel-encoding polynucleotide includes coding regions (e.g., exons) and non-coding regions (e.g., introns or splicing sites). In some embodiments, the target base (e.g., a C base) is located in the coding region (e.g., an exon) of the ion channel-encoding polynucleotide (e.g., the ion channel gene locus). In some embodiments, the conversion of a base in the coding region results in an amino acid change in the ion channel protein sequence, i.e., a mutation. In some embodiments, editing the ion channel-encoding polynucleotide results in a loss-of-function mutant (e.g., for SCN9A). In some embodiments, editing the ion channel-encoding polynucleotide results in a gain-of-function mutant (e.g., for SCN11A).

In some embodiments, the target base is located in a non-coding region of the ion channel-encoding polynucleotide, e.g., in an intron or a splicing site. In some embodiments, a target base is located in a splicing site and the editing of such target base causes alternative splicing of the ion channel mRNA. In some embodiments, the alternative splicing leads to loss-of-function ion-channel mutants. In some embodiments, the alternative splicing leads to the introduction of a premature stop codon in an ion channel mRNA, resulting in truncated and/or unstable ion channel proteins. In some embodiments, ion channel mutants that are defective in folding are produced.

In some embodiments, the activity of a loss-of-function ion channel variant may be reduced by at least 20%, at least 30%, at least 40%, at least 50%, at least 60%, at least 70%, at least 80%, at least 90%, at least 99%, or more. In some embodiments, the loss-of-function ion channel variant has no more than 50%, no more than 40%, no more than 30%, no more than 20%, no more than 10%, no more than 5%, no more than 1%, or less activity compared to a wild type ion channel protein.

In some embodiments, the activity of a gain-of-function ion channel variant may be elevated by at least 20%, at least 30%, at least 40%, at least 50%, at least 60%, at least 70%, at least 80%, at least 90%, at least 99%, or more. In some embodiments, the loss-of-function ion channel variant has no more than 50%, no more than 40%, no more than 30%, no more than 20%, no more than 10%, no more than 5%, no more than 1%, or less activity compared to a wild type ion channel protein.

To edit the ion channel-encoding polynucleotide gene, the ion channel-encoding nucleotide may contacted with a genome-editing agent (e.g., a programmable nuclease or a nucleobase editor), wherein the genome-editing agent binds to its target sequence and edits the target site. For example, the genome-editing agent (e.g., a nucleobase editor) may be expressed in a cell where editing is desired (e.g., a DRG neuron), to thereby allow contacting of the ion channel gene with the agent. In some embodiments, the binding of the genome editing agent (e.g., a nucleobase editor) to its target sequence in the ion channel-encoding polynucleotide is mediated by a guide nucleotide sequence, e.g., a guide RNA (gRNA). The guide nucleotide sequence is designed to be complementary to one of the strands of the target sequence in the ion channel-encoding polynucleotide. The guide nucleotide sequence may be engineered to guide the nucleobase editor to any target base (e.g., target bases listed in Table 2) in an ion channel gene (e.g., SCN9A), provided that a PAM is located 3′ of the target base. In some embodiments, the guide nucleotide sequence is co-expressed with the programmable nuclease or nucleobase editor in a cell where editing is desired (e.g., a DRG neuron). In some embodiments, a programmable nuclease or a nucleobase editor in complex with a gRNA is delivered to a cell where editing is desired (e.g., a DRG neuron). Strategies of editing the ion channel genes using nucleobase editors are provided.

### Codon Change

Using the nucleobase editors, an amino acid codon may be converted to a different codon via deamination of a target base within the codon. For example, in some embodiments, a cytosine (C) base is converted to a thymine (T) base via deamination by a nucleobase editor comprising a cytosine deaminase domain (e.g., APOBEC1 or AID). It is worth noting that during a C to T change via deamination (e.g., by a cytosine deaminase such as APOBEC1 or AID), the cytosine is first converted to a uridine (U), leading to a G:U mismatch. The G:U mismatch is then converted by DNA repair machinery and replication pathways to T:A pair, thus introducing the thymine at the position of the original cytosine. In some embodiments, conversion of a base in an amino acid codon may lead to a change of the amino acid the codon encodes. Cytosine deaminases are capable of converting a cytosine (C) base to a thymine (T) base via deamination. Thus, it is envisioned that, for amino acid codons containing a C base, the C base may be directly converted to T. For example, codon (CTC) for leucine may be changed to a TTC (phenylalanine) codon via the deamination of the first C on the coding strand. For amino acid codons that contain a guanine (G) base, a C base is present on the complementary strand; and the G base may be converted to an adenosine (A) via the deamination of the C on the complementary strand. For example, an ATG (Met/M) codon may be converted to a ATA (lie/I) codon via the deamination of the third C on the complementary strand. In some embodiments, two C to T changes are required to convert a codon to a different codon. Non-limiting examples of possible mutations that may be made (e.g., in the ion channel-encoding polynucleotide) by the nucleobase editors of the present disclosure are summarized in Table 1.

TABLE 1
Exemplary Codon Changes via Base Editing

| Target codon | Base-editing reaction (s) | Edited codon |
|---|---|---|
| CTT (Leu/L) | 1st base C to T on coding strand | TTT (Phe/F) |
| CTC (Leu/L) | 1st base C to T on coding strand | TTC (Phe/F) |
| ATG (Met/M) | 3rd base C to T on complementary strand | ATA (Ile/I) |
| GTT (Val/V) | 1st base C to T on complementary stand | ATT (Ile/I) |
| GTA (Val/V) | 1st base C to T on complementary stand | ATA (Ile/I) |
| GTC (Val/V) | 1st base C to T on complementary strand | ATC (Ile/I) |
| GTG (Val/V) | 1st base C to T on complementary strand | ATG (Met/M) |
| TCT (Ser/S) | 2nd base C to T on coding strand | TTT (Phe/F) |
| TCC (Ser/S) | 2nd base C to T on coding strand | TTC (Phe/F) |
| TCA (Ser/S) | 2nd base C to T on coding strand | TTA (Leu/L) |
| TCG (Ser/S) | 2nd base C to T on coding strand | TTG (Leu/L) |
| AGT (Ser/S) | 2nd base C to T on complementary strand | AAT (Asp/N) |
| AGC (Ser/S) | 2nd base C to T on complementary strand | AAC (Aps/N) |
| CCT (Pro/P) | 1st base C to T on coding strand | TCT (Ser/S) |
| CCC (Pro/P) | 1st base C to T on coding strand | TCC (Ser/S) |
| CCA (Pro/P) | 1st base C to T on coding strand | TCA (Ser/S) |
| CCG (Pro/P) | 1st base C to T on coding strand | TCG (Ser/S) |
| CCT (Pro/P) | 2nd base C to T on coding strand | CTT (Leu/L) |
| CCC (Pro/P) | 2nd base C to T on coding strand | CTC (Leu/L) |
| CCA (Pro/P) | 2nd base C to T on coding strand | CTA (Leu/L) |
| CCG (Pro/P) | 2nd base C to T on coding strand | CTG (Leu/L) |
| ACT (Thr/T) | 2nd base C to T on coding strand | ATT (Leu/L) |
| ACC (Thr/T) | 2nd base C to T on coding strand | ATC (Leu/L) |
| ACA (Thr/T) | 2nd base C to T on coding strand | ATA (Leu/L) |
| ACG (Thr/T) | 2nd base C to T on coding strand | ATG (Met/M) |
| GCT (Ala/A) | 2nd base C to T on coding strand | GTT (Val/V) |
| GCC (Ala/A) | 2nd base C to T on coding strand | GTC (Val/V) |
| GCA (Ala/A) | 2nd base C to T on coding strand | GTA (Val/V) |
| GCG (Ala/A) | 2nd base C to T on coding strand | GTG (Val/V) |
| GCT (Ala/A) | 1st base C to T on complementary stand | ACT (Thr/T) |
| GCC (Ala/A) | 1st base C to T on complementary stand | ACC (Thr/T) |
| GCA (Ala/A) | 1st base C to T on complementary stand | ACA (Thr/T) |
| GCG (Ala/A) | 1st base C to T on complementary stand | ACG (Thr/T) |
| CAT (His/H) | 1st base C to T on complementary stand | TAT (Tyr/Y) |
| CAC (His/H) | 1st base C to T on complementary stand | TAC (Tyr/Y) |
| GAT (Asp/D) | 1st base C to T on complementary stand | AAT (Asp/N) |
| GAC (Asp/D) | 1st base C to T on complementary stand | AAC (Asp/N) |
| GAA (Glu/E) | 1st base C to T on complementary stand | AAA (Lys/K) |
| GAG (Glu/E) | 1st base C to T on complementary stand | AAG (Lys/K) |
| TGT (Cys/C) | 2nd base C to T on complementary stand | TAT (Tyr/Y) |
| TGC (Cys/C) | 2nd base C to T on complementary stand | TAC (Tyr/Y) |
| CGT (Arg/R) | 1st base C to T on coding strand | TGT (Cys/C) |
| CGC (Arg/R) | 1st base C to T on coding strand | TGC (Cys/C) |
| AGA (Arg/R) | 2nd base C to T on complementary stand | AAA (Lys/K) |
| AGG (Arg/R) | 2nd base C to T on complementary stand | AAG (Lys/K) |
| CGG (Arg/R) | 2nd base C to T on complementary stand | CAG (Gln/Q) |
| CGG (Arg/R) | 1st base C to T on coding strand | TGG (Trp/W) |
| GGT (Gly/G) | 2nd base C to T on complementary stand | GAT (Asp/D) |
| GGC (Gly/G) | 2nd base C to T on complementary stand | GAC (Asp/D) |
| GGA (Gly/G) | 2nd base C to T on complementary stand | GAA (Glu/E) |
| GGG (Gly/G) | 2nd base C to T on complementary stand | GAG (Glu/E) |
| GGT (Gly/G) | 1st base C to T on complementary stand | AGT (Ser/S) |
| GGC (Gly/G) | 1st base C to T on complementary stand | AGC (Ser/S) |
| GGA (Gly/G) | 1st base C to T on complementary stand | AGA (Arg/R) |
| GGG (Gly/G) | 1st base C to T on complementary stand | AGG (Arg/R) |

In some embodiments, nucleobase editor is guided by a guide nucleotide sequence to its target sequence that it binds. In some embodiments, the guide nucleotide sequence is a gRNA sequence. An gRNA typically comprises a tracrRNA framework allowing for Cas9 binding, and a guide sequence, which confers sequence specificity to fusion proteins disclosed herein. In some embodiments, the guide RNA comprises a structure 5′-[guide sequence]-guuuuagagcuagaaauagcaaguuaaaauaaaggcuaguccguuaucaacuugaaaaaguggcaccgagucggugcuuuuu-3′ (SEQ ID NO: 338), wherein the guide sequence comprises a sequence that is complementary to the target sequence. The guide sequence is typically about 20 nucleotides long. For example, the guide sequence may be 15-25 nucleotides long. In some embodiments, the guide sequence is 15, 16, 17, 18, 19, 20, 21, 22, 23, 24, or 25 nucleotides long. Such suitable guide RNA sequences typically comprise guide sequences that are complementary to a nucleic sequence within 50 (e.g., 50, 45, 40, 35, 30, 25, 20, 15, or 10) nucleotides upstream or downstream of the target nucleotide to be edited.

In some embodiments, at least 1 mutation is introduced into the ion channel-encoding polynucleotide. In some embodiments, at least 2, 3, 4, 5, 6, 7, 8, 9, 10 or more mutations are introduced into the ion channel-encoding polynucleotide.

Target sites for nucleobase editors in the SCN9A gene encoding the ion channel NaV1.7 are provided in Table 2. The mutations presented herein are for illustration purpose only and are not meant to be limiting.

TABLE 2
Exemplary NaV1.7 (SNA9A) Protective Loss-of-Function Mutations
via Codon Changes

| | | | Program- | | | | |
|---|---|---|---|---|---|---|---|
| | | | mable | SEQ | | | |
| Residue | Codon | Resulting | guide-RNA | ID | | gRNA size | BE |
| Change | Change | Codon(s) | sequence | NOs | (PAM) | (C edited) | type^{a} |
| P5L/S/F | CCT | YYT | GAUGGCAAUG | 339 | (CAG) | 20 (C14/15) | SpBE3 |
| | | | UUGCCUCCCC | | | | |
| P5L/S/F | CCN | YYN | AUGGCAAUGU | 340 | (AGG) | 20 (C13/14) | SpBE3 |
| | | | UGCCUCCCCC | | | | |
| P5/6L/S/F | CCN | YYN | UGGCAAUGUU | 341 | (GGAC) | 20 (C12/13) | VQR- |
| | | | GCCUCCCCCA | | | | SpBE3 |
| P5/6L/S/F | CCN | YYN | UGUUGCCUCC | 342 | (CAG) | 20 (C6/7) | SpBE3 |
| | | | CCCAGGACCU | | | | |
| P5/6/7L/S/F | CCN | YYN | GUUGCCUCCC | 343 | (AGAG) | 20 (C5/6) | EQR- |
| | | | CCAGGACCUC | | | | SpBE3 |
| P5/6/7L7S/F | CCN | YYN | UUGCCUCCCC | 344 | (GAG) | 20 (C4/5) | SpBE3 |
| | | | CAGGACCUCA | | | | |
| P35L/S/F | CCC | YYC | AAAAUCAAAG | 345 | (AAG) | 20 (C14/15) | SpBE3 |
| | | | GAACCCAAAG | | | | |
| P35L/S/F | CCC | YYC | AAAUCAAAGG | 346 | (AGAA) | 20 (C13/14) | VQR- |
| | | | AACCCAAAGA | | | | SpBE3 |
| P35L/S/F | CCC | YYC | CAAAGGAACC | 347 | (AAG) | 20 (C9/10) | SpBE3 |
| | | | CAAAGAAGAA | | | | |
| P35LVS/F | CCC | YYC | AAAGGAACCC | 348 | (AGAA) | 20 (C8/9) | VQR- |
| | | | AAAGAAGAAA | | | | SpBE3 |
| P35L/S/F | CCC | YYC | GGAACCCAAA | 349 | (AAG) | 20 (C5/6) | SpBE3 |
| | | | GAAGAAAAGA | | | | |
| P35L/S/F | CCC | YYC | GAACCCAAAG | 350 | (AGAT) | 20 (C4/5) | VQR- |
| | | | AAGAAAAGAA | | | | SpBE3 |
| P35L7S/F | CCC | YYC | CCCAAAGAAG | 351 | (TGAT) | 20 (C1/2) | VQR- |
| | | | AAAAGAAAGA | | | | SpBE3 |
| P35L/S/F | CCC | YYC | AGGAACCCAA | 352 | (AAAGAT) | 20 (C6/7) | KKH- |
| | | | AGAAGAAAAG | | | | SaBE3 |
| P35L/S/F | CCC | YYC | AACCCAAAGA | 353 | (GATGAT) | 20 (C3/4) | KKH- |
| | | | AGAAAAGAAA | | | | SaBE3 |
| P35LVS/F | CCC | YYC | GAAAAUCAAA | 354 | (GAAGAAA) | 20 (C15/16) | St1BE3 |
| | | | GGAACCCAAA | | | | |
| P35LVS/F | CCC | YYC | UCAAAGGAAC | 355 | (AAAGAAA) | 20 (C10/11) | St1BE3 |
| | | | CCAAAGAAGA | | | | |
| P47L/S/F | CCA | YYA | GAUGAAGAAG | 356 | (AAG) | 20 (C13/14) | SpBE3 |
| | | | CCCCAAAGCC | | | | |
| P47L/S/F | CCA | YYA | GAAGAAGCCC | 357 | (CAG) | 20 (C10/11) | SpBE3 |
| | | | CAAAGCCAAG | | | | |
| P47L/S/F | CCA | YYA | AAGAAGCCCC | 358 | (AGTG) | 20 (C9/10) | VQR- |
| | | | AAAGCCAAGC | | | | SpBE3 |
| P47L/S/F | CCA | YYA | GAAGCCCCAA | 359 | (TGAC) | 20 (C7/8) | VQR- |
| | | | AGCCAAGCAG | | | | SpBE3 |
| P47/49L7S/F | CCA | YYA | CCCAAAGCCA | 360 | (TGG) | 20 (C2/3/ | SpBE3 |
| | | | AGCAGUGACU | | | 8/9) | |
| P47/49L/S/F | CCA | YYA | CCAAAGCCAA | 361 | (GGAA) | 20 (C1/2/ | VQR- |
| | | | GCAGUGACUU | | | 7/8) | SpBE3 |
| P47L/S/F | CCA | YYA | AUGAAGAAGC | 362 | (AGCAGT) | 20 (C12/13) | KKH- |
| | | | CCCAAAGCCA | | | | SaBE3 |
| P49LVS/F | CCA | YYA | AAAGCCAAGC | 363 | (AAG) | 20 (C5/6) | SpBE3 |
| | | | AGUGACUUGG | | | | |
| P49L/S/F | CCA | YYA | CCAAGCAGUG | 364 | (TGG) | 20 (C1/2) | SpBE3 |
| | | | ACUUGGAAGC | | | | |
| P60L/S/F | CCC | YYC | AAACAGCUGC | 365 | (TGG) | 20 (C10/11) | SpBE3 |
| | | | CCUUCAUCUA | | | | |
| P60LVS/F | CCC | YYC | AACAGCUGCC | 366 | (GGG) | 20 (C9/10) | SpBE3 |
| | | | CUUCAUCUAU | | | | |
| P60LVS/F | CCC | YYC | ACAGCUGCCC | 367 | (GGG) | 20 (C8/9) | SpBE3 |
| | | | UUCAUCUAUG | | | | |
| P60L/S/F | CCC | YYC | CAGCUGCCCU | 368 | (GGAC) | 20 (C7/8) | VQR- |
| | | | UCAUCUAUGG | | | | SpBE3 |
| P60L7S/F | CCC | YYC | AAACAGCUGC | 369 | (TGGGG) | 20 (C10/11) | St3BE3 |
| | | | CCUUCAUCUA | | | | |
| P67/8L/S/F | CCT | YYT | UGGGGACAUU | 370 | (TGG) | 20 (Cl 1 -15) | SpBE3 |
| | | | CCUCCCGGCA | | | | |
| P67/8L7S/F | CCT | YYT | GGGGACAUUC | 371 | (GGTG) | 20 (C10-14) | VQR- |
| | | | CUCCCGGCAU | | | | SpBE3 |
| P67/8L/S/F | CCT | YYT | CAUUCCUCCC | 372 | (CAG) | 20 (C5-9) | SpBE3 |
| | | | GGCAUGGUGU | | | | |
| P67/8L/S/F | CCT | YYT | AUUCCUCCCG | 373 | (AGAG) | 20 (C4-8) | EQR* |
| | | | GCAUGGUGUC | | | | SpBE3 |
| P67/8L7S/F | CCT | YYT | UUCCUCCCGG | 374 | (GAG) | 20 (C3-7) | SpBE3 |
| | | | CAUGGUGUCA | | | | |
| P67L/S/F | CCT | YYT | UAUGGGGACA | 375 | (CATGGT) | 20 (C13-17) | KKH- |
| | | | UUCCUCCCGG | | | | SaBE3 |
| P67L/S/F | CCT | YYT | UGGGGACAUU | 376 | (TGGTG) | 20 (C11-15) | St3BE3 |
| | | | CCUCCCGGCA | | | | |
| P74L/S/F | CCC | YYC | CAUGGUGUCA | 377 | (AGG) | 20 (C14/15) | SpBE3 |
| | | | GAGCCCCUGG | | | | |
| P74LVS/F | CCC | YYC | AUGGUGUCAG | 378 | (GGAC) | 20 (C13/14) | VQR- |
| | | | AGCCCCUGGA | | | | SpBE3 |
| P74L/S/F | CCC | YYC | GUCAGAGCCC | 379 | (TGG) | 20 (C8/9) | SpBE3 |
| | | | CUGGAGGACU | | | | |
| P74L/S/F | CCC | YYC | UCAGAGCCCC | 380 | (GGAC) | 20 (C7/8) | VQR- |
| | | | UGGAGGACUU | | | | SpBE3 |
| P80L7S/F | CCC | YYC | GGACUUGGAC | 381 | (CAG) | 20 (C11/12) | SpBE3 |
| | | | CCCUACUAUG | | | | |
| P80L/S/F | CCC | YYC | GACUUGGACC | 382 | (AGAC) | 20 (C10/11) | VQR- |
| | | | CCUACUAUGC | | | | SpBE3 |
| P80LVS/F | CCC | YYC | ACCCCUACUA | 383 | (AAG) | 20 (C3/4) | SpBE3 |
| | | | UGCAGACAAA | | | | |
| P80L/S/F | CCC | YYC | CCCCUACUAU | 387 | (AGG) | 20 (C2/3) | SpBE3 |
| | | | GCAGACAAAA | | | | |
| P80LVS/F | CCC | YYC | CCCUACUAUG | 388 | (GGTG) | 20 (C1/2) | VQR- |
| | | | CAGACAAAAA | | | | SpBE3 |
| P80LVS/F | CCC | YYC | GACCCCUACU | 389 | (AAAGGT) | 20 (C4/5) | KKH- |
| | | | AUGCAGACAA | | | | SaBE3 |
| P80L/S/F | CCC | YYC | CCCCUACUAU | 390 | (AGGTG) | 20 (C2/3) | St3BE3 |
| | | | GCAGACAAAA | | | | |
| P111L7S/F | CCT | YYT | CUUUCUCCUU | 391 | (AAG) | 20 (C7/8) | SpBE3 |
| | | | UCAGUCCUCU | | | | |
| P111L/S/F | CCT | YYT | UUUCUCCUUU | 392 | (AGAA) | 20 (C6/7) | VQR- |
| | | | CAGUCCUCUA | | | | SpBE3 |
| P111L7S/F | CCT | YYT | UCUCCUUUCA | 393 | (AAG) | 20 (C4/5) | SpBE3 |
| | | | GUCCUCUAAG | | | | |
| P111/4L/S/F | CCT | YYT | CUCCUUUCAG | 394 | (AGAA) | 20 | VQR- |
| | | | UCCUCUAAGA | | | (C3/4/12) | SpBE3 |
| P111L/S/F | CCT | YYT | UCUCCUUUCA | 395 | (AAGAAT) | 20 (C4/5) | SaBE3 |
| | | | GUCCUCUAAG | | | | |
| P111L/S/F | CCT | YYT | GCUUUCUCCU | 396 | (TAAGAAG) | 20 (C8/9) | St1BE3 |
| | | | UUCAGUCCUC | | | | |
| P111L/S/F | CCT | YYT | UUCUCCUUUC | 397 | (GAAGAAT) | 20 (C5/6) | St1BE3 |
| | | | AGUCCUCUAA | | | | |
| P114L/S/F | CCT | YYT | CCUCUAAGAA | 398 | (TAAGAT) | 20 | KKH- |
| | | | GAAUAUCUAU | | | (C1/2/12) | SaBE3 |
| C134Y | TGC | TAC | AGUGCACAUG | 399 | (TGAA) | 20 (C5) | VQR- |
| | | | AUGAGCAUGC | | | | SpBE3 |
| C134Y | TGC | TAC | GUCAGAAUAG | 400 | (GAG) | 20 (C13) | SpBE3 |
| | | | UGCACAUGAU | | | | |
| C134Y | TGC | TAC | CACAUGAUGA | 401 | (TAAGGT) | 20 (C1) | KKH- |
| | | | GCAUGCUGAA | | | | SaBE3 |
| C134Y | TGC | TAC | UAGUGCACAU | 402 | (CTGAAT) | 20 (C6) | SaBE3 |
| | | | GAUGAGCAUG | | | | |
| C140Y | TGC | TAC | AAUAUGCAGU | 403 | (AGTG) | 20 (C7) | VQR- |
| | | | UUGUCAGAAU | | | | SpBE3 |
| C140Y | TGC | TAC | AAAUAUGCAG | 404 | (TAG) | 20 (C8) | SpBE3 |
| | | | UUUGUCAGAA | | | | |
| C140Y | TGC | TAC | UCAUAAAUAU | 405 | (AGAA) | 20 (C12) | VQR- |
| | | | GCAGUUUGUC | | | | SpBE3 |
| C140Y | TGC | TAC | GUCAUAAAUA | 406 | (CAG) | 20 (C13) | SpBE3 |
| | | | UGCAGUUUGU | | | | |
| C140Y | TGC | TAC | CAGUUUGUCA | 407 | (CATGAT) | 20 (C1) | KKH- |
| | | | GAAUAGUGCA | | | | SaBE3 |
| C140Y | TGC | TAC | AUAAAUAUGC | 408 | (AATAGT) | 20 (C10) | KKH- |
| | | | AGUUUGUCAG | | | | SaBE3 |
| C140Y | TGC | TAC | GUCAUAAAUA | 409 | (CAGAAT) | 20 (C13) | SaBE3 |
| | | | UGCAGUUUGU | | | | |
| C140Y | TGC | TAC | GGUCAUAAAU | 410 | (TCAGAAT) | 20 (C14) | St1BE3 |
| | | | AUGCAGUUUG | | | | |
| P148L7S/F | CCA | YYA | CCAUGAAUAA | 411 | (TGG) | 20 (C12/13) | SpBE3 |
| | | | CCCACCGGAC | | | | |
| P148L7S/F | CCA | YYA | CAUGAAUAAC | 412 | (GGAC) | 20 (C11/12) | VQR- |
| | | | CCACCGGACU | | | | SpBE3 |
| P148L/S/F | CCA | YYA | AUAACCCACC | 413 | (AAAAAT) | 20 (C6-10) | KKH- |
| | | | GGACUGGACC | | | | SaBE3 |
| P149US/F | CCG | YYG | ACCGGACUGG | 414 | (TCGAGT) | 20 (C2/3) | SaBE3 |
| | | | ACCAAAAAUG | | | | |
| G161R | GGA | ARR | AAAGUAUAUA | 415 | (AGTG) | 20 (C13) | VQR- |
| | | | UUCCAGUAAA | | | | SpBE3 |
| G161R | GGA | ARR | UCAAAAGUAU | 416 | (AAAAGT) | 20 (C16) | KKH- |
| | | | AUAUUCCAGU | | | | SaBE3 |
| G179R | GGA | ARR | CUACACAGAA | 417 | (AGG) | 20 (C-1) | SpBE3 |
| | | | GCCUCUUGCA | | | | |
| G179R | GGA | ARR | CCUACACAGA | 418 | (AAG) | 20 (C1) | SpBE3 |
| | | | AGCCUCUUGC | | | | |
| G179R | GGA | ARR | AAGUGAAUUC | 419 | (AAG) | 20 (C12) | SpBE3 |
| | | | UCCUACACAG | | | | |
| G179R | GGA | ARR | AAAAGUGAAU | 420 | (AGAA) | 20 (C14) | VQR- |
| | | | UCUCCUACAC | | | | SpBE3 |
| G179R | GGA | ARR | CCUACACAGA | 421 | (AAGGAT) | 20 (C1) | SaBE3 |
| | | | AGCCUCUUGC | | | | |
| G179R | GGA | ARR | AGAAAAGUGA | 422 | (ACAGAAG) | 20 (C16) | St1BE3 |
| | | | AUUCUCCUAC | | | | |
| P187L/S/F | CCG | YYG | UUCUUCGUGA | 423 | (TGG) | 20 (C12/13) | SpBE3 |
| | | | CCCGUGGAAC | | | | |
| P187L/S/F | CCG | YYG | UCGUGACCCG | 424 | (TGG) | 20 (C8/9) | SpBE3 |
| | | | UGGAACUGGC | | | | |
| P187L/S/F | CCG | YYG | CGUGACCCGU | 425 | (GGAT) | 20 (C7/8) | VQR- |
| | | | GGAACUGGCU | | | | SpBE3 |
| P187L/S/F | CCG | YYG | UUCGUGACCC | 426 | (CTGGAT) | 20 (C9/10) | SaBE3 |
| | | | GUGGAACUGG | | | | |
| P229L/S/F | CCA | YYA | UUUCUGUAAU | 427 | (AAG) | 20 (C12/13) | SpBE3 |
| | | | CCCAGGUAAG | | | | |
| P229L7S/F | CCA | YYA | AAUCCCAGGU | 428 | (TGG) | 20 (C5/6) | SpBE3 |
| | | | AAGAAGUAAU | | | | |
| P229L/S/F | CCA | YYA | AUCCCAGGUA | 429 | (GGTG) | 20 (C4/5) | VQR- |
| | | | AGAAGUAAUU | | | | SpBE3 |
| P229L/S/F | CCA | YYA | CCCAGGUAAG | 430 | (TGTG) | 20 (C2/3) | VQR- |
| | | | AAGUAAUUGG | | | | SpBE3 |
| P229US/F | CCA | YYA | UAUUUCUGUA | 431 | (AGAAGT) | 20 (C14/15) | KKH- |
| | | | AUCCCAGGUA | | | | SaBE3 |
| P229L/S/F | CCA | YYA | UUCUGUAAUC | 432 | (AGTAAT) | 20 (C11/12) | KKH- |
| | | | CCAGGUAAGA | | | | SaBE3 |
| P229US/F | CCA | YYA | GUAAUCCCAG | 433 | (ATTGGT) | 20 (C7/8) | KKH- |
| | | | GUAAGAAGUA | | | | SaBE3 |
| P229L/S/F | CCA | YYA | AAUCCCAGGU | 434 | (TGGTG) | 20 (C5/6) | St3BE3 |
| | | | AAGAAGUAAU | | | | |
| G236R | GGG | ARR | CCUACAAUUG | 435 | (TGAA) | 20 (C1) | VQR- |
| | | | UCUUCAGGCC | | | | SpBE3 |
| G236R | GGG | ARR | AAGCCCCUAC | 436 | (AGG) | 20 (C6) | SpBE3 |
| | | | AAUUGUCUUC | | | | |
| G236R | GGG | ARR | AAAGCCCCUA | 437 | (CAG) | 20 (C7) | SpBE3 |
| | | | CAAUUGUCUU | | | | |
| G236R | GGG | ARR | CUACAAUUGU | 438 | (GAAAAT) | 20 (C-1) | KKH- |
| | | | CUUCAGGCCU | | | | SaBE3 |
| C255Y | TGT | TAT | ACAGAACACA | 439 | (TGAC) | 20 (C2) | VQR- |
| | | | GUCAGGAUCA | | | | SpBE3 |
| C255Y | TGT | TAT | ACUCAGACAG | 440 | (GGAT) | 20 (C8) | VQR- |
| | | | AACACAGUCA | | | | SpBE3 |
| C255Y | TGT | TAT | CACUCAGACA | 441 | (AGG) | 20 (C9) | SpBE3 |
| | | | GAACACAGUC | | | | |
| C255Y | TGT | TAT | ACACUCAGAC | 442 | (CAG) | 20 (C10) | SpBE3 |
| | | | AGAACACAGU | | | | |
| C255Y | TGT | TAT | ACACUCAGAC | 443 | (CAGGAT) | 20 (C10) | SaBE3 |
| | | | AGAACACAGU | | | | |
| G263R | GGA | ARR | CAAUUAGUGC | 444 | (AGAC) | 20 (C-1) | VQR- |
| | | | AAACACACUC | | | | SpBE3 |
| G263R | GGA | ARR | CCAAUUAGUG | 445 | (CAG) | 20 (C1) | SpBE3 |
| | | | CAAACACACU | | | | |
| C275Y | TGT | TAT | UUCGAAAACA | 446 | (AGG) | 20 (C9) | SpBE3 |
| | | | UUUAUGCUUC | | | | |
| C275Y | TGT | TAT | UUUCGAAAAC | 447 | (CAG) | 20 (C10) | SpBE3 |
| | | | AUUUAUGCUU | | | | |
| C275Y | TGT | TAT | AUUUCGAAAA | 448 | (TCAGGT) | 20 (C11) | KKH- |
| | | | CAUUUAUGCU | | | | SaBE3 |
| C315Y | TGT | TAT | CCACAAAGGA | 449 | (GGAT) | 20 (C4) | VQR- |
| | | | GAGCAUCUUU | | | | SpBE3 |
| C315Y | TGT | TAT | ACCACAAAGG | 450 | (TGG) | 20 (C5) | SpBE3 |
| | | | AGAGCAUCUU | | | | |
| C315Y | TGT | TAT | UGUGCUGAAA | 451 | (GAG) | 20 (C14) | SpBE3 |
| | | | CCACAAAGGA | | | | |
| C315Y | TGT | TAT | AACCACAAAG | 452 | (TTGGAT) | 20 (C6) | SaBE3 |
| | | | GAGAGCAUCU | | | | |
| C324Y | TGT | TAT | ACACUGACUA | 453 | (AGAA) | 20 (C2) | VQR- |
| | | | CACACGAGAA | | | | SpBE3 |
| C324Y | TGT | TAT | GACACUGACU | 454 | (AAG) | 20 (C3) | SpBE3 |
| | | | ACACACGAGA | | | | |
| C324Y | TGT | TAT | CUGGACACUG | 455 | (AGAA) | 20 (C6) | VQR- |
| | | | ACUACACACG | | | | SpBE3 |
| C324Y | TGT | TAT | UCUGGACACU | 456 | (GAG) | 20 (C7) | SpBE3 |
| | | | GACUACACAC | | | | |
| C324Y | TGT | TAT | GGACACUGAC | 457 | (AAAGAAC) | 20 (C4) | St1BE3 |
| | | | UACACACGAG | | | | |
| C324Y | TGT | TAT | CUCUGGACAC | 458 | (CGAGAAA) | 20 (C8) | St1BE3 |
| | | | UGACUACACA | | | | |
| C325Y | TGT | TAT | CUCUGGACAC | 459 | (CGAG) | 20 (C8) | EQR- |
| | | | UGACUACACA | | | | SpBE3 |
| P325L/S/F | CCA | YYA | AGUGUCCAGA | 460 | (TGTG) | 20 (C6/7) | VQR- |
| | | | GGGGUACACC | | | | SpBE3 |
| P325L/S/F | CCA | YYA | UGUCCAGAGG | 461 | (TGTG) | 20 (C4/5) | VQR- |
| | | | GGUACACCUG | | | | SpBE3 |
| P325LVS/F | CCA | YYA | UCCAGAGGGG | 462 | (TGAA) | 20 (C2/3) | VQR- |
| | | | UACACCUGUG | | | | SpBE3 |
| P325L/S/F | CCA | YYA | CCAGAGGGGU | 463 | (GAAAAT) | 20 (C1/2) | KKH- |
| | | | ACACCUGUGU | | | | SaBE3 |
| C330Y | TGT | TAT | AGGUGUACCC | 464 | (TGAC) | 20 (C-1) | VQR- |
| | | | CUCUGGACAC | | | | SpBE3 |
| C330Y | TGT | TAT | UCACACAGGU | 465 | (GGAC) | 20 (C6) | VQR- |
| | | | GUACCCCUCU | | | | SpBE3 |
| C330Y | TGT | TAT | UUCACACAGG | 466 | (TGG) | 20 (C7) | SpBE3 |
| | | | UGUACCCCUC | | | | |
| P337L/S/F | CCT | YYT | AUUGGCAGAA | 467 | (TGG) | 20 (C13/14) | SpBE3 |
| | | | ACCCUGAUUA | | | | |
| P337L/S/F | CCT | YYT | AAACCCUGAU | 468 | (CGAG) | 20 (C5/6) | EQR- |
| | | | UAUGGCUACA | | | | SpBE3 |
| P337L/S/F | CCT | YYT | AACCCUGAUU | 469 | (GAG) | 20 (C4/5) | SpBE3 |
| | | | AUGGCUACAC | | | | |
| P532L/S/F | CCC | YYC | UACCCCCAAU | 470 | (CCAAAT) | 20 (C5/6) | KKH- |
| | | | CAGGUACCAC | | | | SaBE3 |
| P536L/S/F | CCA | YYA | UGCAGUCACC | 471 | (CGTG) | 20 (C9/10) | VQR- |
| | | | ACUCAGCAUU | | | | SpBE3 |
| P536L/S/F | CCA | YYA | CAGUCACCAC | 472 | (TGG) | 20 (C7/8) | SpBE3 |
| | | | UCAGCAUUCG | | | | |
| P591L/S/F | CCC | YYC | GCUCACUGUU | 473 | (AGAC) | 20 (C15/16) | VQR- |
| | | | UGUGCCCCAC | | | | SpBE3 |
| P591L/S/F | CCC | YYC | UGUUUGUGCC | 474 | (CAG) | 20 (C9/10) | SpBE3 |
| | | | CCACAGACCC | | | | |
| P591L7S/F | CCC | YYC | GUUUGUGCCC | 475 | (AGG) | 20 (Ca8/9) | SpBE3 |
| | | | CACAGACCCC | | | | |
| P591L/S/F | CCC | YYC | UUUGUGCCCC | 476 | (GGAG) | 20 (C7/8) | EQR- |
| | | | ACAGACCCCA | | | | SpBE3 |
| P591L/S/F | CCC | YYC | UUGUGCCCCA | 477 | (GAG) | 20 (C6/7) | SpBE3 |
| | | | CAGACCCCAG | | | | |
| P591L/S/F | CCC | YYC | UGUGCCCCAC | 478 | (AGCG) | 20 (C5/6) | VRER- |
| | | | AGACCCCAGG | | | | SpBE3 |
| P591L/S/F | CCC | YYC | UGCCCCACAG | 479 | (CGAC) | 20 | VQR- |
| | | | ACCCCAGGAG | | | (C3/4/12) | SpBE3 |
| P591L/S/F | CCC | YYC | GUUUGUGCCC | 480 | (AGGAG) | 20 (C8/9) | St3BE3 |
| | | | CACAGACCCC | | | | |
| P594L7S/F | CCC | YYC | CACAGACCCC | 481 | (CAG) | 20 (C7/8) | SpBE3 |
| | | | AGGAGCGACG | | | | |
| P594LVS/F | CCC | YYC | AGACCCCAGG | 482 | (CAG) | 20 (C4/5) | SpBE3 |
| | | | AGCGACGCAG | | | | |
| P594L7S/F | CCC | YYC | ACAGACCCCA | 483 | (AGCAGT) | 20 (C6/7) | KKH- |
| | | | GGAGCGACGC | | | | SaBE3 |
| P609/10L/S/ | CCA | YYA | UAGGUCCCCA | 484 | (CGG) | 20 (C8-12) | SpBE3 |
| F | | | CCAAUGCUGC | | | | |
| P609/10L/S/ | CCA | YYA | AGGUCCCCAC | 485 | (GGTG) | 20 (C7-11) | VQR- |
| F | | | CAAUGCUGCC | | | | SpBE3 |
| P609/10L/S/ | CCA | YYA | GUCCCCACCA | 486 | (TGAA) | 20 (C5-9) | VQR- |
| F | | | AUGCUGCCGG | | | | SpBE3 |
| P609/10L/S/ | CCA | YYA | CCACCAAUGC | 487 | (CGG) | 20 (C1-4) | SpBE3 |
| F | | | UGCCGGUGAA | | | | |
| P609/10L/S/ | CCA | YYA | AGUACCUCCC | 488 | (GCCGGT) | 20 (C10-14) | KKH- |
| F | | | CACCAAUGCU | | | | SaBE3 |
| P609/10L7S/ | CCA | YYA | UAGGUCCCCA | 489 | (CGGTG) | 20 (C8-12) | St3BE3 |
| F | | | CCAAUGCUGC | | | | |
| P610L/S/F | CCA | YYA | CACCAAUGCU | 490 | (GGG) | 20 (C3/4) | SpBE3 |
| | | | GCCGGUGAAC | | | | |
| P610L/S/F | CCA | YYA | ACCAAUGCUG | 491 | (GGAA) | 20 (C2/3) | VQR- |
| | | | CCGGUGAACG | | | | SpBE3 |
| P613L7S/F | CCG | YYG | CACCAAUGCU | 492 | (GGG) | 20 (C12/13) | SpBE3 |
| | | | GCCGGUGAAC | | | | |
| P613L7S/F | CCG | YYG | ACCAAUGCUG | 493 | (GGAA) | 20 (C11/12) | VQR- |
| | | | CCGGUGAACG | | | | SpBE3 |
| P613L/S/F | CCG | YYG | CCGGUGAACG | 494 | (CAG) | 20 (C1/2) | SpBE3 |
| | | | GGAAAAUGCA | | | | |
| P613L7S/F | CCG | YYG | CCAAUGCUGC | 495 | (GAAAAT) | 20 (C10/11) | KKH- |
| | | | CGGUGAACGG | | | | SaBE3 |
| P640L/S/F | CCC | YYC | CCCUCAUGCU | 496 | (CAG) | 20 (C12/13) | SpBE3 |
| | | | CCCCAAUGGA | | | | |
| P640US/F | CCC | YYC | CCCCAAUGGA | 497 | (CAG) | 20 (C2/3) | SpBE3 |
| | | | CAGCUUCUGC | | | | |
| P640L7S/F | CCC | YYC | CCCAAUGGAC | 498 | (AGAG) | 20 (C1/2) | EQR- |
| | | | AGCUUCUGCC | | | | SpBE3 |
| P640L/S/F | CCC | YYC | CCCAAUGGAC | 499 | (AGAGGT) | 20 (C1/2) | KKH- |
| | | | AGCUUCUGCC | | | | SaBE3 |
| P646L7S/F | CCA | YYA | GCUUCUGCCA | 500 | (TAG) | 20 (C8/9) | SpBE3 |
| | | | GAGGUGAUAA | | | | |
| P646L7S/F | CCA | YYA | CUUCUGCCAG | 501 | (AGAT) | 20 (C7/8) | VQR- |
| | | | AGGUGAUAAU | | | | SpBE3 |
| P646L/S/F | CCA | YYA | UGCCAGAGGU | 502 | (AAG) | 20 (C3/4) | SpBE3 |
| | | | GAUAAUAGAU | | | | |
| P646L7S/F | CCA | YYA | GCCAGAGGUG | 503 | (AGG) | 20 (C2/3) | SpBE3 |
| | | | AUAAUAGAUA | | | | |
| P646L/S/F | CCA | YYA | GGACAGCUUC | 504 | (GATAAT) | 20 (C13/14) | KKH- |
| | | | UGCCAGAGGU | | | | SaBE3 |
| P646L/S/F | CCA | YYA | AGCUUCUGCC | 505 | (ATAGAT) | 20 (C9/10) | KKH- |
| | | | AGAGGUGAUA | | | | SaBE3 |
| P683L7S/F | CCC | YYC | AUGCUGAAUG | 506 | (CAG) | 20 (C13/14) | SpBE3 |
| | | | AUCCCAACCU | | | | |
| P683L/S/F | CCC | YYC | UGCUGAAUGA | 507 | (AGAC) | 20 (02/13) | VQR- |
| | | | UCCCAACCUC | | | | SpBE3 |
| P683LVS/F | CCC | YYC | UGAAUGAUCC | 508 | (CAG) | 20 (C9/10) | SpBE3 |
| | | | CAACCUCAGA | | | | |
| P683L7S/F | CCC | YYC | GAAUGAUCCC | 509 | (AGAG) | 20 (C8/9) | EQR- |
| | | | AACCUCAGAC | | | | SpBE3 |
| P683L/S/F | CCC | YYC | AAUGAUCCCA | 510 | (GAG) | 20 (C7/8) | SpBE3 |
| | | | ACCUCAGACA | | | | |
| P683L7S/F | CCC | YYC | AUGAUCCCAA | 511 | (AGAG) | 20 (C6/7) | EQR- |
| | | | CCUCAGACAG | | | | SpBE3 |
| P683L7S/F | CCC | YYC | UGAUCCCAAC | 512 | (GAG) | 20 (C5/6) | SpBE3 |
| | | | CUCAGACAGA | | | | |
| P683L/S/F | CCC | YYC | GAUCCCAACC | 513 | (AGCAAT) | 20 (C4/5) | KKH- |
| | | | UCAGACAGAG | | | | SaBE3 |
| P711US/F | CCA | YYA | CAGACAAAAA | 514 | (GGTG) | 20 (04/15) | VQR- |
| | | | UGUCCACCUU | | | | SpBE3 |
| P711US/F | CCA | YYA | GACAAAAAUG | 515 | (TGG) | 20 (02/13) | SpBE3 |
| | | | UCCACCUUGG | | | | |
| P711/2L/S/F | CCA | YYA | AAAUGUCCAC | 516 | (CAG) | 20 (C7-11) | SpBE3 |
| | | | CUUGGUGGUA | | | | |
| P711/2L/S/F | CCA | YYA | AAUGUCCACC | 517 | (AGAT) | 20 (C6-10) | VQR- |
| | | | UUGGUGGUAC | | | | SpBE3 |
| P711LVS/F | CCA | YYA | CAGACAAAAA | 518 | (GGTGGT) | 20 (04/15) | KKH- |
| | | | UGUCCACCUU | | | | SaBE3 |
| P711/2L/S/F | CCA | YYA | AAAAUGUCCA | 519 | (ACAGAT) | 20 (C8-12) | KKH- |
| | | | CCUUGGUGGU | | | | SaBE3 |
| P711L7S/F | CCA | YYA | CCAGACAAAA | 520 | (TGGTG) | 20 (C15/16) | St3BE3 |
| | | | AUGUCCACCU | | | | |
| P728L/S/F | CCA | YYA | GAAUUGCUCU | 521 | (TAAAAT) | 20 (C11/12) | KKH- |
| | | | CCAUAUUGGA | | | | SaBE3 |
| P728L/S/F | CCA | YYA | UCCAUAUUGG | 522 | (AAAAGT) | 20 (C2/3) | KKH- |
| | | | AUAAAAUUCA | | | | SaBE3 |
| P744L/S/F | CCT | YYT | AUUGUAAUGG | 523 | (AGAT) | 20 (C13/14) | VQR- |
| | | | AUCCUUUUGU | | | | SpBE3 |
| P744L/S/F | CCT | YYT | UUAUUGUAAU | 524 | (GTAGAT) | 20 (C15/16) | KKH- |
| | | | GGAUCCUUUU | | | | SaBE3 |
| P744L/S/F | CCT | YYT | AUGGAUCCUU | 525 | (TGCAAT) | 20 (C7/8) | KKH- |
| | | | UUGUAGAUCU | | | | SaBE3 |
| C753V | TGC | TAC | CUAUGCAAAU | 526 | (AGAT) | 20 (C6) | VQR- |
| | | | GGUAAUUGCA | | | | SpBE3 |
| C753Y | TGC | TAC | ACUAUGCAAA | 527 | (AAG) | 20 (C7) | SpBE3 |
| | | | UGGUAAUUGC | | | | |
| C753Y | TGC | TAC | AACUAUGCAA | 528 | (CAAGAT) | 20 (C8) | KKH- |
| | | | AUGGUAAUUG | | | | SaBE3 |
| P767L/S/F | CCA | YYA | AUGGAACACC | 529 | (TGAG) | 20 (C13/14) | EQR- |
| | | | ACCCAAUGAC | | | | SpBE3 |
| P767L/S/F | CCA | YYA | UGGAACACCA | 530 | (GAG) | 20 (C12/13) | SpBE3 |
| | | | CCCAAUGACU | | | | |
| P767L/S/F | CCA | YYA | GGAACACCAC | 531 | (AGG) | 20 (C11/12) | SpBE3 |
| | | | CCAAUGACUG | | | | |
| P767US/F | CCA | YYA | GAACACCACC | 532 | (GGAA) | 20 (C10/11) | VQR- |
| | | | CAAUGACUGA | | | | SpBE3 |
| P767L/S/F | CCA | YYA | GGAACACCAC | 533 | (AGGAAT) | 20 (C11/12) | SaBE3 |
| | | | CCAAUGACUG | | | | |
| P767LVS/F | CCA | YYA | ACCCAAUGAC | 534 | (AAAAAT) | 20 (C3/4) | KKH- |
| | | | UGAGGAAUUC | | | | SaBE3 |
| G779R | GGA | ARR | UCCUAUAGCA | 535 | (TGAA) | 20 (C2) | VQR- |
| | | | AGUACAUUUU | | | | SpBE3 |
| G779R | GGA | ARR | CUUACCAAAU | 536 | (AAG) | 20 (C13) | SpBE3 |
| | | | UUCCUAUAGC | | | | |
| G779R | GGA | ARR | UUCCUAUAGC | 537 | (TTGAAT) | 20 (C3) | SaBE3 |
| | | | AAGUACAUUU | | | | |
| G779R | GGA | ARR | GACUUACCAA | 538 | (GCAAGT) | 20 (C15) | KKH- |
| | | | AUUUCCUAUA | | | | SaBE3 |
| G785R | GGA | ARR | UUCCAGUAAA | 539 | (AGAA) | 20 (C3) | VQR- |
| | | | GACCUAAGUG | | | | SpBE3 |
| G785R | GGA | ARR | AUUCCAGUAA | 540 | (GAG) | 20 (C4) | SpBE3 |
| | | | AGACCUAAGU | | | | |
| G785R | GGA | ARA | AAGAUUCCAG | 541 | (AGTG) | 20 (C7) | VQR- |
| | | | UAAAGACCUA | | | | SpBE3 |
| G785R | GGA | ARA | AAAGAUUCCA | 542 | (AAG) | 20 (C8) | SpBE3 |
| | | | GUAAAGACCU | | | | |
| G785R | GGA | ARA | AGCUGCAAAG | 543 | (AGAC) | 20 (C14) | VQR- |
| | | | AUUCCAGUAA | | | | SpBE3 |
| G785R | GGA | ARA | CCAGUAAAGA | 544 | (AAAAAT) | 20 (C1) | KKH- |
| | | | CCUAAGUGAG | | | | SaBE3 |
| G785R | GGA | ARA | GCAAAGAUUC | 545 | (CTAAGT) | 20 (C10) | KKH- |
| | | | CAGUAAAGAC | | | | SaBE3 |
| G785R | GGA | ARA | GAUUCCAGUA | 546 | (TGAGAAA) | 20 (C5) | St1BE3 |
| | | | AAGACCUAAG | | | | |
| G786R | GGA | ARR | GAUUCCAGUA | 547 | (TGAG) | 20 (C5) | EQR- |
| | | | AAGACCUAAG | | | | SpBE3 |
| P800US/F | CCA | YYA | GGAUCCAUAU | 548 | (AAG) | 20 (C5/6) | SpBE3 |
| | | | GAGUAUUUCC | | | | |
| P800L/S/F | CCA | YYA | UCCAUAUGAG | 549 | (TAG) | 20 (C2/3) | SpBE3 |
| | | | UAUUUCCAAG | | | | |
| P800L/S/F | CCA | YYA | CCAUAUGAGU | 550 | (AGG) | 20 (C1/2) | SpBE3 |
| | | | AUUUCCAAGU | | | | |
| P800L7S/F | CCA | YYA | AUGGAUCCAU | 551 | (CCAAGT) | 20 (C7/8) | KKH- |
| | | | AUGAGUAUUU | | | | SaBE3 |
| G830R | GGA | ARA | CCUUCCACAU | 552 | (GAG) | 20 (Cl) | SpBE3 |
| | | | CUGCUAGAAA | | | | |
| G830R | GGA | ARA | AUCCUUCCAC | 553 | (AAG) | 20 (C3) | SpBE3 |
| | | | AUCUGCUAGA | | | | |
| G830R | GGA | ARA | ACAAUCCUUC | 554 | (AGAA) | 20 (C6) | VQR- |
| | | | CACAUCUGCU | | | | SpBE3 |
| G830R | GGA | ARA | GACAAUCCUU | 555 | (TAG) | 20 (C7) | SpBE3 |
| | | | CCACAUCUGC | | | | |
| G830R | GGA | ARA | UGACAAUCCU | 556 | (CTAGAAA) | 20 (C8) | St1BE3 |
| | | | UCCACAUCUG | | | | |
| G831R | GGA | ARA | UCCUUCCACA | 557 | (AGAG) | 20 (C2) | EQR- |
| | | | UCUGCUAGAA | | | | SpBE3 |
| P850US/F | CCA | YYA | CUGGCCAACA | 558 | (TGAT) | 20 (C5/6) | VQR- |
| | | | UUGAACAUGC | | | | SpBE3 |
| P850LVS/F | CCA | YYA | UCCUGGCCAA | 559 | (GCTGAT) | 20 (C7/8) | KKH- |
| | | | CAUUGAACAU | | | | |
| | | | | | | | SaBE3 |
| P850L/S/F | CCA | YYA | CCAACAUUGA | 560 | (TAAGAT) | 20 (C1/2) | KKH- |
| | | | ACAUGCUGAU | | | | SaBE3 |
| C895Y | TGT | TAT | AUUCUUUGUA | 561 | (AAG) | 20 (C-1) | SpBE3 |
| | | | GCUCUUACCA | | | | |
| C895Y | TGT | TAT | UCUUGCAGAC | 562 | (TAG) | 20 (C12) | SpBE3 |
| | | | ACAUUCUUUG | | | | |
| C897Y | TGC | TAC | UCUUGCAGAC | 563 | (TAG) | 20 (C6) | SpBE3 |
| | | | ACAUUCUUUG | | | | |
| C903Y | TGT | TAT | ACAGUCAUCA | 564 | (AGAC) | 20 (C2) | VQR- |
| | | | UUGAUCUUGC | | | | SpBE3 |
| C903Y | TGT | TAT | UACAGUCAUC | 565 | (CAG) | 20 (C3) | SpBE3 |
| | | | AUUGAUCUUG | | | | |
| C903Y | TGT | TAT | UGGGAGCGUA | 566 | (TGAT) | 20 (C11) | VQR- |
| | | | CAGUCAUCAU | | | | SpBE3 |
| P906L/S/F | CCA | YYA | UACGCUCCCA | 567 | (TGAA) | 20 (C8/9) | VQR- |
| | | | CGGUGGCACA | | | | SpBE3 |
| P906L/S/F | CCA | YYA | CUCCCACGGU | 568 | (CGAC) | 20 (C4/5) | VQR- |
| | | | GGCACAUGAA | | | | SpBE3 |
| C925Y | TGT | TAT | ACAGCACGCG | 569 | (AGG) | 20 (C2) | SpBE3 |
| | | | GAACACAAUC | | | | |
| C925Y | TGT | TAT | CACAGCACGC | 570 | (CAG) | 20 (C3) | SpBE3 |
| | | | GGAACACAAU | | | | |
| C925Y | TGT | TAT | CCACUCUCCA | 571 | (GGAA) | 20 (C13) | VQR- |
| | | | CACAGCACGC | | | | SpBE3 |
| C925Y | TGT | TAT | UCCACUCUCC | 572 | (CGG) | 20 (C14) | SpBE3 |
| | | | ACACAGCACG | | | | |
| C925Y | TGT | TAT | UAUCCACUCU | 573 | (CGCG) | 20 (C16) | VRER- |
| | | | CCACACAGCA | | | | SpBE3 |
| C925Y | TGT | TAT | UCUCCACACA | 574 | (CACAAT) | 20 (C9) | KKH- |
| | | | GCACGCGGAA | | | | SaBE3 |
| C935Y | TGT | TAT | GACCUCCAUA | 575 | (TGG) | 20 (C13) | SpBE3 |
| | | | CAGUCCCACA | | | | |
| C935Y | TGT | TAT | GCGACCUCCA | 576 | (CATGGT) | 20 (C15) | KKH- |
| | | | UACAGUCCCA | | | | SaBE3 |
| C944Y | TGC | TAC | AAGGCACAUA | 577 | (CGAC) | 20 (C5) | VQR- |
| | | | GCUUGACCAG | | | | SpBE3 |
| C944Y | TGC | TAC | AUAAGGCACA | 578 | (AGCG) | 20 (C7) | VRER- |
| | | | UAGCUUGACC | | | | SpBE3 |
| C944Y | TGC | TAC | AAUAAGCCAC | 579 | (CAG) | 20 (C8) | SpBE3 |
| | | | AUAGCUUGAC | | | | |
| C944Y | TGC | TAC | AAACAAUAAG | 580 | (TGAC) | 20 (C12) | VQR- |
| | | | GCACAUAGCU | | | | SpBE3 |
| G955R | GGA | ARA | CACCAGGUUU | 581 | (TGAC) | 20 (C11) | VQR- |
| | | | CCAAUGACCA | | | | SpBE3 |
| P983L7S/F | CCT | YYT | ACCCUGAUGC | 582 | (CAG) | 20 (C3/4) | SpBE3 |
| | | | AAACAACCUC | | | | |
| P983L7S/F | CCT | YYT | CCCUGAUGCA | 583 | (AGAT) | 20 (C2/3) | VQR- |
| | | | AACAACCUCC | | | | SpBE3 |
| P983L/S/F | CCT | YYT | GACCCUGAUG | 584 | (CCAGAT) | 20 (C4/5) | KKH- |
| | | | CAAACAACCU | | | | SaBE3 |
| P1018LVS/F | CCA | YYA | UCCAAAAAGC | 585 | (CAG) | 20 (C10/11) | SpBE3 |
| | | | CAAAGAUUUC | | | | |
| P1018LVS/F | CCA | YYA | CCAAAAAGCC | 586 | (AGG) | 20 (C9/10) | SpBE3 |
| | | | AAAGAUUUCC | | | | |
| P1018LVS/F | CCA | YYA | CAAAAAGCCA | 587 | (GGG) | 20 (C8/9) | SpBE3 |
| | | | AAGAUUUCCA | | | | |
| P1018L7S/F | CCA | YYA | AAAAAGCCAA | 588 | (GGAG) | 20 (C7/8) | EQR- |
| | | | AGAUUUCCAG | | | | SpBE3 |
| P1018L7S/F | CCA | YYA | AAAAGCCAAA | 589 | (GAG) | 20 (C6/7) | SpBE3 |
| | | | GAUUUCCAGG | | | | |
| P1018LVS/F | CCA | YYA | AAAGCCAAAG | 590 | (AGAT) | 20 (C5/6) | VQR- |
| | | | AUUUCCAGGG | | | | SpBE3 |
| P1018LVS/F | CCA | YYA | CCAAAGAUUU | 591 | (AAG) | 20 (C1/2) | SpBE3 |
| | | | CCAGGGAGAU | | | | |
| P1018LVS/F | CCA | YYA | AAAAAGCCAA | 592 | (GGAGAT) | 20 (C7/8) | KKH- |
| | | | AGAUUUCCAG | | | | SaBE3 |
| P1018LVS/F | CCA | YYA | CAAAAAGCCA | 593 | (GGGAG) | 20 (C8/9) | St3BE3 |
| | | | AAGAUUUCCA | | | | |
| P1083L7S/F | CCC | YYC | UAUUCACAAU | 594 | (CAG) | 20 (C11/12) | SpBE3 |
| | | | CCCAGCCUCA | | | | |
| P1083LVS/F | CCC | YYC | AUUCACAAUC | 595 | (AGTG) | 20 (C10/11) | VQR- |
| | | | CCAGCCUCAC | | | | SpBE3 |
| P1083L7S/F | CCC | YYC | UCACAAUCCC | 596 | (TGAC) | 20 (C8/9) | VQR- |
| | | | AGCCUCACAG | | | | SpBE3 |
| P1083LVS/F | CCC | YYC | CAAUCCCAGC | 597 | (CAG) | 20 (C5/6) | SpBE3 |
| | | | CUCACAGUGA | | | | |
| P1083L/S/F | CCC | YYC | AAUCCCAGCC | 598 | (AGTG) | 20 (C4/5) | VQR- |
| | | | UCACAGUGAC | | | | SpBE3 |
| P1083L7S/F | CCC | YYC | UUUAUUCACA | 599 | (CACAGT) | 20 (C13/14) | KKH- |
| | | | AUCCCAGCCU | | | | SaBE3 |
| P1083L/S/F | CCC | YYC | CACAAUCCCA | 600 | (GACAGT) | 20 (C7/8) | KKH- |
| | | | GCCUCACAGU | | | | SaBE3 |
| P1083L/S/F | CCC | YYC | CCCAGCCUCA | 601 | (GCCAAT) | 20 (C1/2) | KKH- |
| | | | CAGUGACAGU | | | | SaBE3 |
| P1090US/F | CCA | YYA | GUGACAGUGC | 602 | (TGG) | 20 (C10/11) | SpBE3 |
| | | | CAAUUGCACC | | | | |
| P1090LVS/F | CCA | YYA | UGACACUCCC | 603 | (GGG) | 20 (C9/10) | SpBE3 |
| | | | AAUUGCACCU | | | | |
| P1090L7S/F | CCA | YYA | GACAGUGCCA | 604 | (GGG) | 20 (C8/9) | SpBE3 |
| | | | AUUGCACCUG | | | | |
| P1090L/S/F | CCA | YYA | ACACUGCCAA | 605 | (GGAA) | 20 (C7/8) | VQR- |
| | | | UUGCACCUGG | | | | SpBE3 |
| P1090/3L/S/ | CCA | YYA | CCAAUUGCAC | 606 | (CGAT) | 20 | VQR- |
| F | | | CUGGGGAAUC | | | (C1/2/10/11 | SpBE3 |
| | | | | | | ) | |
| P1090LVS/F | CCA | YYA | GACAGUGCCA | 607 | (GGGAAT) | 20 (C8/9) | SaBE3 |
| | | | AUUGCACCUG | | | | |
| P1090/3L/S/ | CCA | YYA | UGCCAAUUGC | 608 | (TCCGAT) | 20 | KKH- |
| F | | | ACCUGGGGAA | | | (C3/4/13) | SaBE3 |
| P1090L7S/F | CCA | YYA | GUGACAGUGC | 609 | (TGGGG) | 20 (C10/11) | St3BE3 |
| | | | CAAUUGCACC | | | | |
| P1093LVS/F | CCT | YYT | UGCACCUGGG | 610 | (TGG) | 20 (C5/6) | SpBE3 |
| | | | GAAUCCGAUU | | | | |
| P1093L/S/F | CCT | YYT | GCACCUGGGG | 611 | (GGAA) | 20 (C4/5) | VQR- |
| | | | AAUCCGAUUU | | | | SpBE3 |
| P1093LVS/F | CCT | YYT | CACCUGGGGA | 612 | (GAAAAT) | 20 (C3/4) | KKH- |
| | | | AUCCGAUUUG | | | | SaBE3 |
| P1133US/F | CCT | YYT | ACAGUUGAUA | 613 | (TGG) | 20 (C13/14) | SpBE3 |
| | | | ACCCUUUGCC | | | | |
| P1133L/S/F | CCT | YYT | CAGUUGAUAA | 614 | (GGAG) | 20 (Cl 2/13) | EQR- |
| | | | CCCUUUGCCU | | | | SpBE3 |
| P1133LVS/F | CCT | YYT | AGUUGAUAAC | 615 | (GAG) | 20 (C11/12) | SpBE3 |
| | | | CCUUUGCCUG | | | | |
| P1133L7S/F | CCT | YYT | GUUGAUAACC | 616 | (AGAA) | 20 (C10/11) | VQR- |
| | | | CUUUGCCUGG | | | | SpBE3 |
| P1133/5L/S/ | CCT | YYT | UGAUAACCCU | 617 | (AAG) | 20 (C8-14) | SpBE3 |
| F | | | UUGCCUGGAG | | | | |
| P1133/5L/S/ | CCT | YYT | GAUAACCCUU | 618 | (AGG) | 20 (C7-14) | SpBE3 |
| F | | | UGCCUGGAGA | | | | |
| P1133/5L/S/ | CCT | YYT | AUAACCCUUU | 619 | (GGAG) | 20 (C6-13) | EQR- |
| F | | | GCCUGGAGAA | | | | SpBE3 |
| P1133/5L/S/ | CCT | YYT | UAACCCUUUG | 620 | (GAG) | 20 (C5-12) | SpBE3 |
| F | | | CCUGGAGAAG | | | | |
| P1133/5L/S/ | CCT | YYT | AACCCUUUGC | 621 | (AGAA) | 20 (C4-11) | VQR- |
| F | | | CUGGAGAAGG | | | | SpBE3 |
| P1133/5L/S/ | CCT | YYT | CCCUUUGCCU | 622 | (AAG) | 20 (C2-9) | SpBE3 |
| F | | | GGAGAAGGAG | | | | |
| P1133/5L/S/ | CCT | YYT | CCUUUGCCUG | 623 | (AGAA) | 20 (C1-8) | VQR- |
| F | | | GAGAAGGAGA | | | | SpBE3 |
| P1133/5L/S/ | CCT | YYT | ACAGUUGAUA | 624 | (TGGAG) | 20 (C13/14) | St3BE3 |
| F | | | ACCCUUUGCC | | | | |
| P1133/5L/S/ | CCT | YYT | CAGUUGAUAA | 625 | (GGAGAAG) | 20 (C12/13) | SHBE3 |
| F | | | CCCUUUGCCU | | | | |
| P1133/5L/S/ | CCT | YYT | GAUAACCCUU | 626 | (AGGAG) | 20 (C7-14) | St3BE3 |
| F | | | UGCCUGGAGA | | | | |
| P1133/5L/S/ | CCT | YYT | AUAACCCUUU | 627 | (GGAGAAG) | 20 (C6-13) | St1BE3 |
| F | | | GCCUGGAGAA | | | | |
| P1133/5L/S/ | CCT | YYT | ACCCUUUGCC | 628 | (GAAGAAG) | 20 (C3-10) | St1BE3 |
| F | | | UGGAGAAGGA | | | | |
| P1135L/S/F | CCT | YYT | UUUGCCUGGA | 629 | (AAG) | 20 (C5/6) | SpBE3 |
| | | | GAAGGAGAAG | | | | |
| P1135LVS/F | CCT | YYT | GCCUGGAGAA | 630 | (CAG) | 20 (C2/3) | SpBE3 |
| | | | GGAGAAGAAG | | | | |
| P1135IVS/F | CCT | YYT | CCUGGAGAAG | 631 | (AGAG) | 20 (C1/2) | EQR- |
| | | | GAGAAGAAGC | | | | SpBE3 |
| P1145L7S/F | CCT | YYT | GAGGCUGAAC | 632 | (CGAT) | 20 (C10/11) | VQR- |
| | | | CUAUGAAUUC | | | | SpBE3 |
| P1145US/F | CCT | YYT | GCUGAACCUA | 633 | (TGAG) | 20 (C7/8) | EQR- |
| | | | UGAAUUCCGA | | | | SpBE3 |
| P1145L7S/F | CCT | YYT | CUGAACCUAU | 634 | (GAG) | 20 (C6/7) | SpBE3 |
| | | | GAAUUCCGAU | | | | |
| P1145LVS/F | CCT | YYT | ACCUAUGAAU | 635 | (CAG) | 20 (C2/3) | SpBE3 |
| | | | UCCGAUGAGC | | | | |
| P1145LVS/F | CCT | YYT | CCUAUGAAUU | 636 | (AGAG) | 20 (C1/2) | EQR- |
| | | | CCGAUGAGCC | | | | SpBE3 |
| P1145LVS/F | CCT | YYT | CAGAGGCUGA | 637 | (TCCGAT) | 20 (C12/13) | KKH- |
| | | | ACCUAUGAAU | | | | SaBE3 |
| P1151L/S/F | CCA | YYA | UGAGCCAGAG | 638 | (CAG) | 20 (C5/6) | SpBE3 |
| | | | GCCUGUUUCA | | | | |
| P1151LVS/F | CCA | YYA | GAGCCAGAGG | 639 | (AGAT) | 20 (C4/5) | VQR- |
| | | | CCUGUUUCAC | | | | SpBE3 |
| P1151LVS/F | CCA | YYA | CCAGAGGCCU | 640 | (TGG) | 20 (C1/2) | SpBE3 |
| | | | GUUUCACAGA | | | | |
| P1151LVS/F | CCA | YYA | AUGAGCCAGA | 641 | (ACAGAT) | 20 (C6/7) | KKH- |
| | | | GGCCUGUUUC | | | | SaBE3 |
| P1151LVS/F | CCA | YYA | AGCCAGAGGC | 642 | (GATGGT) | 20 (C3/4) | KKH- |
| | | | CUGUUUCACA | | | | SaBE3 |
| C1154Y | TGT | TAT | AAACAGGCCU | 643 | (GGAA) | 20 (C6) | VQR- |
| | | | CUGGCUCAUC | | | | SpBE3 |
| C1154Y | TGT | TAT | GAAACAGGCC | 644 | (CGG) | 20 (C7) | SpBE3 |
| | | | UCUGGCUCAU | | | | |
| C1154Y | TGT | TAT | CCAUCUGUGA | 645 | (TGG) | 20 (C15) | SpBE3 |
| | | | AACAGGCCUC | | | | |
| C1154Y | TGT | TAT | GAAACAGGCC | 646 | (CGGAAT) | 20 (C7) | SaBE3 |
| | | | UCUGGCUCAU | | | | |
| C1159Y | TGT | TAT | CAUACACAAC | 647 | (AGAC) | 20 (C7) | VQR- |
| | | | CUGACAAGAA | | | | SpBE3 |
| C1159Y | TGT | TAT | CCAUACACAA | 648 | (AAG) | 20 (C8) | SpBE3 |
| | | | CCUGACAAGA | | | | |
| C1159Y | TGT | TAT | CCUCCAUACA | 649 | (AGAA) | 20 (C11) | VQR- |
| | | | CAACCUGACA | | | | SpBE3 |
| C1159Y | TGT | TAT | ACCUCCAUAC | 650 | (AAG) | 20 (C12) | SpBE3 |
| | | | ACAACCUGAC | | | | |
| C1159Y | TGT | TAT | AACCUCCAUA | 651 | (CAAGAAA) | 20 (C13) | St1BE3 |
| | | | CACAACCUGA | | | | |
| P1285LVS/F | CCC | YYC | UUGGCCCCAU | 652 | (CGG) | 20 (C6/7) | SpBE3 |
| | | | UAAAUCCCUU | | | | |
| P1285LVS/F | CCC | YYC | UGGCCCCAUU | 653 | (GGAC) | 20 (C5/6) | VQR- |
| | | | AAAUCCCUUC | | | | SpBE3 |
| P1297LVS/F | CCT | YYT | AGACCUCUAA | 654 | (TAG) | 20 (C4/5) | SpBE3 |
| | | | GAGCCUUAUC | | | | |
| P1297LVS/F | CCT | YYT | UACCUCUAAG | 655 | (AGAT) | 20 (C3/4) | VOR- |
| | | | AGCCUUAUCU | | | | SpBE3 |
| P1297LVS/F | CCT | YYT | AAGACCUCUA | 656 | (CTAGAT) | 20 (C5/6) | KKH- |
| | | | AGAGCCUUAU | | | | SaBE3 |
| P1319LVS/F | CCT | YYT | AGGAGCAAUU | 657 | (TGAA) | 20 (C11/12) | VQR- |
| | | | CCUUCCAUCA | | | | SpBE3 |
| P1319LVS/F | CCT | YYT | GCAAUUCCUU | 658 | (TGTG) | 20 (C7/8) | VQR- |
| | | | CCAUCAUGAA | | | | SpBE3 |
| P1319LVS/F | CCT | YYT | UAGGAGCAAU | 659 | (ATGAAT) | 20 (C12/13) | SaBE3 |
| | | | UCCUUCCAUC | | | | |
| C1328Y | TGT | TAT | ACACACAAGU | 660 | (TGAT) | 20 (C4) | VQR- |
| | | | AGCACAUUCA | | | | SpBE3 |
| C1328Y | TGT | TAT | AGACACACAA | 661 | (CATGAT) | 20 (C6) | KKH- |
| | | | GUAGCACAUU | | | | SaBE3 |
| G1339R | GGA | ARA | CCAUGAUGCU | 662 | (CAG) | 20 (C-1) | SpBE3 |
| | | | GAAUAUCAGC | | | | |
| G1339R | GGA | ARA | ACUCCCAUGA | 663 | (CAG) | 20 (C4) | SpBE3 |
| | | | UGCUGAAUAU | | | | |
| G1339R | GGA | ARA | CAAAUUUACU | 664 | (TGAA) | 20 (C11) | VQR- |
| | | | CCCAUGAUGC | | | | SpBE3 |
| G1339R | GGA | ARA | CCAUGAUGCU | 665 | (CAGAAT) | 20 (C1) | SaBE3 |
| | | | GAAUAUCAGC | | | | |
| G1339R | GGA | ARA | ACAAAUUUAC | 666 | (CTGAAT) | 20 (C12) | SaBE3 |
| | | | UCCCAUGAUG | | | | |
| G1339R | GGA | ARA | CCCAUGAUGC | 667 | (CCAGAAT) | 20 (C1) | St1BE3 |
| | | | UGAAUAUCAG | | | | |
| C1350Y | TGT | TAT | AAUACACUCA | 668 | (CAG) | 20 (C7) | SpBE3 |
| | | | UAGAACUUGC | | | | |
| P1360LVS/F | CCT | YYT | GUCACGGUUU | 669 | (AAG) | 20 (C11/12) | SpBE3 |
| | | | CCUGCAAGUC | | | | |
| P1360LVS/F | CCT | YYT | GGGUCACGGU | 670 | (TCAAGT) | 20 (C13/14) | KKH- |
| | | | UUCCUGCAAG | | | | SaBE3 |
| P1360LVS/F | CCT | YYT | GGUUUCCUGC | 671 | (CCAAAT) | 20 (C6/7) | KKH- |
| | | | AAGUCAAGUU | | | | SaBE3 |
| P1365LVS/F | CCA | YYA | AGUCAAGUUC | 672 | (CGAA) | 20 (C10/11) | VQR- |
| | | | CAAAUCGUUC | | | | SpBE3 |
| P1365LVS/F | CCA | YYA | AAGUCAAGUU | 673 | (CCGAAT) | 20 (C11/12) | SaBE3 |
| | | | CCAAAUCGUU | | | | |
| C1370Y | TGT | TAT | AUUCGGAACG | 674 | (TGAC) | 20 (C2) | VQR- |
| | | | AUUUGGAACU | | | | SpBE3 |
| C1370V | TGT | TAT | CAAAACAUUC | 675 | (GGAA) | 20 (C8) | VQR- |
| | | | GGAACGAUUU | | | | SpBE3 |
| C1370Y | TGT | TAT | GCAAAACAUU | 676 | (TGG) | 20 (C9) | SpBE3 |
| | | | CGGAACGAUU | | | | |
| C1370Y | TGT | TAT | UAAGGGCAAA | 677 | (CGAT) | 20 (C14) | VQR- |
| | | | ACAUUCGGAA | | | | SpBE3 |
| C1370Y | TGT | TAT | CAUAAGGGCA | 678 | (AACGAT) | 20 (C16) | KKH- |
| | | | AAACAUUCGG | | | | SaBE3 |
| P1425L7S/F | CCC | YYC | GUAGACAAGC | 679 | (TGAA) | 20 (C13/14) | VQR- |
| | | | AGCCCAAAUA | | | | SpBE3 |
| P1425L7S/F | CCC | YYC | AAGCAGCCCA | 680 | (TAG) | 20 (C7/8) | SpBE3 |
| | | | AAUAUGAAUA | | | | |
| G1444R | GGG | ARR | UGACCCAAAG | 681 | (CGAC) | 20 (C5) | VQR- |
| | | | AUGAUAAAGA | | | | SpBE3 |
| G1444R | GGG | ARR | GAAUGACCCA | 682 | (AGAC) | 20 (C8) | VQR- |
| | | | AAGAUGAUAA | | | | SpBE3 |
| G1444R | GGG | ARR | AGAAUGACCC | 683 | (AAG) | 20 (C9) | SpBE3 |
| | | | AAAGAUGAUA | | | | |
| G1444R | GGG | ARR | AGUGAAGAAU | 684 | (TGAT) | 20 (C14) | VQR- |
| | | | GACCCAAAGA | | | | SpBE3 |
| G1444R | GGG | ARR | CCCAAAGAUG | 685 | (CAAAAT) | 20 (C2) | KKH- |
| | | | AUAAAGACGA | | | | SaBE3 |
| S1490F^{b} | TCC | TTY | UGGGGUCCAA | 686 | (AAG) | 20 (C7/8) | SpBE3 |
| | | | GAAGCCACAA | | | | |
| S1490F^{b} | TCC | TTY | GGGUCCAAGA | 687 | (GCCAAT) | 20 (C5/6) | KKH- |
| | | | AGCCACAAAA | | | | SaBE3 |
| P1493/6L/S/ | CCA | YYA | AGCCACAAAA | 688 | (CGAC) | 20 | VQR- |
| F | | | GCCAAUUCCU | | | (C3/4/12) | SpBE3 |
| P1493US/F | CCA | YYA | GGGUCCAAGA | 689 | (GCCAAT) | 20 (C13/14) | KKH- |
| | | | AGCCACAAAA | | | | SaBE3 |
| P1496L7S/F | CCA | YYA | ACAAAAGCCA | 690 | (CAG) | 20 (C8/9) | SpBE3 |
| | | | AUUCUUCGAC | | | | |
| P1496/8L/S/ | CCA | YYA | CAAAAGCCAA | 691 | (AGG) | 20 (C7-14) | SpBE3 |
| F | | | UUCCUCGACC | | | | |
| P1496/8L/S/ | CCA | YYA | AAAAGCCAAU | 692 | (GGG) | 20 (C6-13) | SpBE3 |
| F | | | UCCUCGACCA | | | | |
| P1496/8L/S/ | CCA | YYA | AAAGCCAAUU | 693 | (GGG) | 20 (C5-12) | SpBE3 |
| F | | | CCUCGACCAG | | | | |
| P1496/8L/S/ | CCA | YYA | AAAGCCAAUU | 694 | (GGG) | 20 (C5-12) | SpBE3 |
| F | | | CCUCGACCAG | | | | |
| P1496/8USI | CCA | YYA | CAAAAGCCAA | 695 | (AGGGGT) | 20 (C7-14) | SaBE3 |
| F | | | UUCCUCGACC | | | | |
| P1496L7S/F | CCA | YYA | CAAAAGCCAA | 696 | (AGGGG) | 20 (C7-14) | St3BE3 |
| | | | UUCCUCGACC | | | | |
| P1498/1500 | CCT | YYT | AAUUCCUCGA | 697 | (AAAAAT) | 20 (C5-12) | KKH- |
| L/S/F | | | CCAGGGGUAA | | | | SaBE3 |
| P1500L7S/F | CCA | YYA | AAUUCCUCGA | 698 | (AAAAAT) | 20 (C11/12) | KKH- |
| | | | CCAGGGGUAA | | | | SaBE3 |
| C1526Y | TGT | TAT | GUUGAGACAG | 699 | (TGAT) | 20 (C8) | VQR- |
| | | | AUAAGAACCA | | | | SpBE3 |
| C1526Y | TGT | TAT | UUACCAUGUU | 700 | (AGAA) | 20 (C15) | VQR- |
| | | | GAGACAGAUA | | | | SpBE3 |
| C1526Y | TGT | TAT | GUUACCAUGU | 701 | (AAG) | 20 (C16) | SpBE3 |
| | | | UGAGACAGAU | | | | |
| C1526Y | TGT | TAT | AGACAGAUAA | 702 | (ACTAAT) | 20 (C4) | KKH- |
| | | | GAACCAUGAU | | | | SaBE3 |
| C1526Y | TGT | TAT | AUGUUGAGAC | 703 | (CATGAT) | 20 (C10) | KKH- |
| | | | AGAUAAGAAC | | | | SaBE3 |
| C1526Y | TGT | TAT | GGUUACCAUG | 704 | (TAAGAAC) | 20 (C17) | St1BE3 |
| | | | UUGAGACAGA | | | | |
| G1560R | GGA | ARA | CACACAUUCU | 705 | (GGAT) | 20 (C11) | VQR- |
| | | | CCAGUGAAAA | | | | SpBE3 |
| G1560R | GGA | ARA | GCACACAUUC | 706 | (AGG) | 20 (C12) | SpBE3 |
| | | | UCCAGUGAAA | | | | |
| G1560R | GGA | ARA | AGCACACAUU | 707 | (AAG) | 20 (C13) | SpBE3 |
| | | | CUCCAGUGAA | | | | |
| G1560R | GGA | ARA | AGCACACAUU | 708 | (AAGGAT) | 20 (C13) | SaBE3 |
| | | | CUCCAGUGAA | | | | |
| C1562Y | TGT | TAT | CACACAUUCU | 709 | (GGAT) | 20 (C5) | VQR- |
| | | | CCAGUGAAAA | | | | SpBE3 |
| C1562Y | TGT | TAT | GCACACAUUC | 710 | (AGG) | 20 (C6) | SpBE3 |
| | | | UCCAGUGAAA | | | | |
| C1562Y | TGT | TAT | AGCACACAUU | 711 | (AAG) | 20 (C7) | SpBE3 |
| | | | CUCCAGUGAA | | | | |
| C1562Y | TGT | TAT | UUUUAGCACA | 712 | (TGAA) | 20 (C11) | VQR- |
| | | | CAUUCUCCAG | | | | SpBE3 |
| C1562Y | TGT | TAT | AGUUUUAGCA | 713 | (AGTG) | 20 (C13) | VQR- |
| | | | CACAUUCUCC | | | | SpBE3 |
| C1562Y | TGT | TAT | CAGUUUUAGC | 714 | (CAG) | 20 (C14) | SpBE3 |
| | | | ACACAUUCUC | | | | |
| C1562Y | TGT | TAT | AGCACACAUU | 715 | (AAGGAT) | 20 (CT) | SaBE3 |
| | | | CUCCAGUGAA | | | | |
| C1562Y | TGT | TAT | AUCAGUUUUA | 716 | (TCCAGT) | 20 (C16) | KKH- |
| | | | GCACACAUUC | | | | SaBE3 |
| G1577R | GGA | ARA | CCAUCCUACA | 717 | (AGTG) | 20 (C5) | VQR- |
| | | | GUGAAGUAGU | | | | SpBE3 |
| G1577R | GGA | ARA | UCCAUCCUAC | 718 | (TAG) | 20 (C6) | SpBE3 |
| | | | AGUGAAGUAG | | | | |
| G1577R | GGA | ARA | UAUUCCAUCC | 719 | (TAG) | 20 (C9) | SpBE3 |
| | | | UACAGUGAAG | | | | |
| G1577R | GGA | ARA | AAAUAUUCCA | 720 | (AAG) | 20 (C12) | SpBE3 |
| | | | UCCUACAGUG | | | | |
| G1577R | GGA | ARA | AAAAAUAUUC | 721 | (TGAA) | 20 (C14) | VQR- |
| | | | CAUCCUACAG | | | | SpBE3 |
| G1577R | GGA | ARA | AUUCCAUCCU | 722 | (AGTAGT) | 20 (C8) | KKH- |
| | | | ACAGUGAAGU | | | | SaBE3 |
| G1577R | GGA | ARA | AAUAUUCCAU | 723 | (AGTAGT) | 20 (C11) | KKH- |
| | | | CCUACAGUGA | | | | SaBE3 |
| G1577R | GGA | ARA | AAAAAUAUUC | 724 | (TGAAGT) | 20 (C14) | KKH- |
| | | | CAUCCUACAG | | | | SaBE3 |
| P1606L7S/F | CCT | YYT | UUGUGUCCCC | 725 | (CGAG) | 20 (C9/10) | EQR- |
| | | | UACCCUGUUC | | | | SpBE3 |
| P1606LVS/F | CCT | YYT | UGUGUCCCCU | 726 | (GAG) | 20 (C8/9) | SpBE3 |
| | | | ACCCUGUUCC | | | | |
| P1606L7S/F | CCT | YYT | GUGUCCCCUA | 727 | (AGTG) | 20 (C7/8) | VQR- |
| | | | CCCUGUUCCG | | | | SpBE3 |
| P1606LVS/F | CCT | YYT | GUCCCCUACC | 728 | (TGAT) | 20 (C5/6) | VQR- |
| | | | CUGUUCCGAG | | | | SpBE3 |
| P1606LVS/F | CCT | YYT | UUUGUGUCCC | 729 | (CCGAGT) | 20 (C10/11) | SaBE3 |
| | | | CUACCCUGUU | | | | |
| P1606LVS/F | CCT | YYT | GUGUCCCCUA | 730 | (AGTGAT) | 20 (C7/8) | KKH- |
| | | | CCCUGUUCCG | | | | SaBE3 |
| G1626R | GGA | ARA | CUUUGACUAG | 731 | (CGG) | 20 (C-1) | SpBE3 |
| | | | ACGUAGGAUU | | | | |
| G1626R | GGA | ARA | UGCUCCUUUG | 732 | (GGAT) | 20 (C5) | VQR- |
| | | | ACUAGACGUA | | | | SpBE3 |
| G1626R | GGA | ARA | UUGCUCCUUU | 733 | (AGG) | 20 (C6) | SpBE3 |
| | | | GACUAGACGU | | | | |
| G1626R | GGA | ARA | UUUGCUCCUU | 734 | (TAG) | 20 (CT) | SpBE3 |
| | | | UGACUAGACG | | | | |
| G1626R | GGA | ARA | UCCCCUUUGC | 735 | (AGAC) | 20 (C12) | VQR- |
| | | | UCCUUUGACU | | | | SpBE3 |
| G1626R | GGA | ARA | AUCCCCUUUG | 736 | (TAG) | 20 (C13) | SpBE3 |
| | | | CUCCUUUGAC | | | | |
| G1626R | GGA | ARA | UUUGCUCCUU | 737 | (TAGGAT) | 20 (CT) | SaBE3 |
| | | | UGACUAGACG | | | | |
| G1629R | GGG | ARR | UCCCCUUUGC | 738 | (AGAC) | 20 (C3) | VQR- |
| | | | UCCUUUGACU | | | | SpBE3 |
| G1629R | GGG | ARR | AUCCCCUUUG | 739 | (TAG) | 20 (C4) | SpBE3 |
| | | | CUCCUUUGAC | | | | |
| G1629R | GGG | ARR | GCGGAUCCCC | 740 | (TGAC) | 20 (C8) | VQR- |
| | | | UUUGCUCCUU | | | | SpBE3 |
| P1642LVS/F | CCT | YYT | CUUCCUGCGU | 741 | (CGG) | 20 (C4/5) | SpBE3 |
| | | | UGUUUAACAU | | | | |
| G1662R | GGA | ARA | CAUUCCAAAG | 742 | (TGAA) | 20 (C5) | VQR- |
| | | | AUGGCGUAGA | | | | SpBE3 |
| G1662R | GGA | ARA | GGACAUUCCA | 743 | (AGAT) | 20 (C8) | VQR- |
| | | | AAGAUGGCGU | | | | SpBE3 |
| G1662R | GGA | ARA | UGGACAUUCC | 744 | (TAG) | 20 (C9) | SpBE3 |
| | | | AAAGAUGGCG | | | | |
| G1662R | GGA | ARA | AAGUUGGACA | 745 | (GGCG) | 20 (C13) | VRER- |
| | | | UUCCAAAGAU | | | | SpBE3 |
| G1662R | GGA | ARA | UUGGACAUUC | 746 | (GTAGAT) | 20 (C10) | KKH- |
| | | | CAAAGAUGGC | | | | SaBE3 |
| G1662R | GGA | ARA | AAAGUUGGAC | 747 | (TGGCG) | 20 (C14) | St3BE3 |
| | | | AUUCCAAAGA | | | | |
| C1690Y | TGC | TAC | GCAAAUCAUA | 748 | (AGG) | 20 (C2) | SpBE3 |
| | | | CUGUUGCCAA | | | | |
| C1690Y | TGC | TAC | GGCAAAUCAU | 749 | (AAG) | 20 (C3) | SpBE3 |
| | | | ACUGUUGCCA | | | | |
| C1690Y | TGC | TAC | AGGCAAAUCA | 750 | (AAAGGT) | 20 (C4) | KKH- |
| | | | UACUGUUGCC | | | | SaBE3 |
| P1706LVS/F | CCT | YYT | UUGCUAGCAC | 751 | (CAG) | 20 (C10/11) | SpBE3 |
| | | | CUAUUCUUAA | | | | |
| P1706LVS/F | CCT | YYT | UAGCACCUAU | 752 | (AAG) | 20 (C6/7) | SpBE3 |
| | | | UCUUAACAGU | | | | |
| P1706L7S/F | CCT | YYT | GAUUGCUAGC | 753 | (AACAGT) | 20 (C12/13) | KKH- |
| | | | ACCUAUUCUU | | | | SaBE3 |
| P1712LVS/F | CCA | YYA | UUAACAGUAA | 754 | (TGTG) | 20 (C12-14) | VQR- |
| | | | GCCACCCGAC | | | | SpBE3 |
| P1712L7S/F | CCA | YYA | AACAGUAAGC | 755 | (TGAC) | 20 (C10/11) | VQR- |
| | | | CACCCGACUG | | | | SpBE3 |
| P1712/3L/S/ | CCA | YYA | CCACCCGACU | 756 | (AAAAGT) | 20 (C1-5) | KKH- |
| F | | | GUGACCCAAA | | | | SaBE3 |
| P1713L/S/F | CCC | YYC | ACCCGACUGU | 757 | (AAG) | 20 (C2/3) | SpBE3 |
| | | | GACCCAAAAA | | | | |
| C1715Y | TGT | TAT | AGUCGGGUGG | 758 | (AGAA) | 20 (C-1) | VQR- |
| | | | CUUACUGUUA | | | | SpBE3 |
| C1715Y | TGT | TAT | CAGUCGGGUG | 759 | (AAG) | 20 (C1) | SpBE3 |
| | | | GCUUACUGUU | | | | |
| C1715Y | TGT | TAT | UUUUUUGGGU | 760 | (TGG) | 20 (C13) | SpBE3 |
| | | | CACAGUCGGG | | | | |
| C1715Y | TGT | TAT | CUUUUUUUGG | 761 | (GGTG) | 20 (C15) | VQR- |
| | | | GUCACAGUCG | | | | SpBE3 |
| C1715Y | TGT | TAT | ACUUUUUUUG | 762 | (GGG) | 20 (C16) | SpBE3 |
| | | | GGUCACAGUC | | | | |
| C1715Y | TGT | TAT | CAGUCGGGUG | 763 | (AAGAAT) | 20 (C1) | SaBE3 |
| | | | GCUUACUGUU | | | | |
| C1715Y | TGT | TAT | GAACUUUUUU | 764 | (TCGGGT) | 20 (C18) | SaBE3 |
| | | | UGGGUCACAG | | | | |
| C1715Y | TGT | TAT | ACAGUCGGGU | 765 | (TAAGAAT) | 20 (C2) | St1BE3 |
| | | | GGCUUACUGU | | | | |
| C1715Y | TGT | TAT | ACUUUUUUUG | 766 | (GGGTG) | 20 (C16) | St3BE3 |
| | | | GGUCACAGUC | | | | |
| P1717L/S/F | CCA | YYA | GACCCAAAAA | 767 | (TGG) | 20 (C4/5) | SpBE3 |
| | | | AAGUUCAUCC | | | | |
| P1717US/F | CCA | YYA | ACCCAAAAAA | 768 | (GGAA) | 20 (C3/4) | VQR- |
| | | | AGUUCAUCCU | | | | SpBE3 |
| P1717L7S/F | CCA | YYA | CCAAAAAAAG | 769 | (AAG) | 20 (C1/2) | SpBE3 |
| | | | UUCAUCCUGG | | | | |
| P1717L/S/F | CCA | YYA | ACCCAAAAAA | 770 | (GGAAGT) | 20 (C3/4) | KKH- |
| | | | AGUUCAUCCU | | | | SaBE3 |
| P1722L/S/F | CCT | YYT | AAAAGUUCAU | 771 | (CAG) | 20 (C11/12) | SpBE3 |
| | | | CCUGGAAGUU | | | | |
| P1722L/S/F | CCT | YYT | GUUCAUCCUG | 772 | (TGAA) | 20 (C7/8) | VQR- |
| | | | GAAGUUCAGU | | | | SpBE3 |
| P1722L7S/F | CCT | YYT | UCAUCCUGGA | 773 | (AAG) | 20 (C5/6) | SpBE3 |
| | | | AGUUCAGUUG | | | | |
| P1722L7S/F | CCT | YYT | CAUCCUGGAA | 774 | (AGG) | 20 (C4/5) | SpBE3 |
| | | | GUUCAGUUGA | | | | |
| P1722US/F | CCT | YYT | AUCCUGGAAG | 775 | (GGAG) | 20 (C3/4) | EQR- |
| | | | UUCAGUUGAA | | | | SpBE3 |
| P1722L7S/F | CCT | YYT | UCCUGGAAGU | 776 | (GAG) | 20 (C2/3) | SpBE3 |
| | | | UCAGUUGAAG | | | | |
| P1722L7S/F | CCT | YYT | CCUGCAAGUU | 777 | (ACAC) | 20 (C1/2) | VQR- |
| | | | CACUUCAAGC | | | | SpBE3 |
| P1722L/S/F | CCT | YYT | AAAAAAGUUC | 778 | (TTCAGT) | 20 (C13/14) | KKH- |
| | | | AUCCUGGAAG | | | | SaBE3 |
| P1722L/S/F | CCT | YYT | CAUCCUGGAA | 779 | (AGGAG) | 20 (C4/5) | St3BE3 |
| | | | GUUCAGUUGA | | | | |
| C1730Y | TGT | TAT | UUACCACAGU | 780 | (TGAA) | 20 (C7) | VQR- |
| | | | CUCCUUCAAC | | | | SpBE3 |
| P1733LVS/F | CCA | YYA | GACUGUGGUA | 781 | (TGG) | 20 (C13/14) | SpBE3 |
| | | | ACCCAUCUGU | | | | |
| P1733L/S/F | CCA | YYA | ACUGUGGUAA | 782 | (GGAA) | 20 (C12/13) | VQR- |
| | | | CCCAUCUGUU | | | | SpBE3 |
| P1733L/S/F | CCA | YYA | GACUGUGGUA | 783 | (TGGAAT) | 20 (C13/14) | SaBE3 |
| | | | ACCCAUCUGU | | | | |
| G1736R | GGA | ARA | UUCCAACAGA | 784 | (CAG) | 20 (C3) | SpBE3 |
| | | | UGGGUUACCA | | | | |
| G1736R | GGA | ARA | AGUAGAAUAU | 785 | (GGG) | 20 (C12) | SpBE3 |
| | | | UCCAACAGAU | | | | |
| G1736R | GGA | ARA | AAGUAGAAUA | 786 | (TGG) | 20 (C13) | SpBE3 |
| | | | UUCCAACAGA | | | | |
| G1736R | GGA | ARA | UAUUCCAACA | 787 | (CACAGT) | 20 (C5) | KKH- |
| | | | GAUGGGUUAC | | | | SaBE3 |
| G1736R | GGA | ARA | AAAGUAGAAU | 788 | (ATGGGT) | 20 (C14) | SaBE3 |
| | | | AUUCCAACAG | | | | |
| P1773I7S/F | CCT | YYT | GAAAGUACUG | 789 | (TGAG) | 20 (C13/14) | EQR- |
| | | | AACCUCUGAG | | | | SpBE3 |
| P1773L7S/F | CCT | YYT | AAAGUACUGA | 790 | (GAG) | 20 (C12/13) | SpBE3 |
| | | | ACCUCUGAGU | | | | |
| P1773LVS/F | CCT | YYT | AAGUACUGAA | 791 | (AGG) | 20 (C11/12) | SpBE3 |
| | | | CCUCUGAGUG | | | | |
| P1773L/S/F | CCT | YYT | AGUACUGAAC | 792 | (GGAT) | 20 (C10/11) | VQR- |
| | | | CUCUGAGUGA | | | | SpBE3 |
| P1773L7S/F | CCT | YYT | ACUGAACCUC | 793 | (TGAC) | 20 (C7/8) | VQR- |
| | | | UGAGUGAGGA | | | | |
| | | | | | | | SpBE3 |
| P1773IVS/F | CCT | YYT | CCUCUGAGUG | 794 | (TGAG) | 20 (C1/2) | EQR- |
| | | | AGGAUGACUU | | | | SpBE3 |
| P1773LVS/F | CCT | YYT | AAAGUACUGA | 795 | (GAGGAT) | 20 (C12/13) | SaBE3 |
| | | | ACCUCUGAGU | | | | |
| P1773LVS/F | CCT | YYT | CCUCUGAGUG | 796 | (TGAGAT) | 20 (C1/2) | KKH- |
| | | | AGGAUGACUU | | | | SaBE3 |
| P1791LVS/F | CCC | YYC | AGUUUGAUCC | 797 | (CAG) | 20 (C9/10) | SpBE3 |
| | | | CGAUGCGACC | | | | |
| P1791LVS/F | CCC | YYC | UCCCGAUGCG | 798 | (TAG) | 20 (C2/3) | SpBE3 |
| | | | ACCCAGUUUA | | | | |
| P1791LVS/F | CCC | YYC | CCCGAUGCGA | 799 | (AGAG) | 20 (C1/2) | EQR- |
| | | | CCCAGUUUAU | | | | SpBE3 |
| P1791LVS/F | CCC | YYC | GAAGUUUGAU | 800 | (CCCAGT) | 20 (C11/12) | KKH- |
| | | | CCCGAUGCGA | | | | SaBE3 |
| P1791LVS/F | CCC | YYC | UCCCGAUGCG | 801 | (TAGAGT) | 20 (C2/3) | SaBE3 |
| | | | ACCCAGUUUA | | | | |
| P1811/2LVS/ | CCT | YYT | CCUGGAUCCU | 802 | (TAG) | 20 (C8-12) | SpBE3 |
| F | | | CCUCUUCUCA | | | | |
| P1818LVS/F | CCC | YYC | UCUCAUAGCA | 803 | (AAG) | 20 (C14/15) | SpBE3 |
| | | | AAACCCAACA | | | | |
| P1818LVS/F | CCC | YYC | UAGCAAAACC | 804 | (CAG) | 20 (C9/10) | SpBE3 |
| | | | CAACAAAGUC | | | | |
| P1818LVS/F | CCC | YYC | CUUCUCAUAG | 805 | (CAAAGT) | 20 (C16/17) | KKH- |
| | | | CAAAACCCAA | | | | SaBE3 |
| P1829LVS/F | CCC | YYC | GCCAUGGAUC | 806 | (TAG) | 20 (C13/14) | SpBE3 |
| | | | UGCCCAUGGU | | | | |
| P1829LVS/F | CCC | YYC | CCAUGGAUCU | 807 | (AGTG) | 20 (C12/13) | VQR- |
| | | | GCCCAUGGUU | | | | SpBE3 |
| P1829LVS/F | CCC | YYC | AUGGAUCUGC | 808 | (TGG) | 20 (C10/11) | SpBE3 |
| | | | CCAUGGUUAG | | | | |
| P1829IVS/F | CCC | YYC | UGGAUCUGCC | 809 | (GGTG) | 20 (C9/10) | VQR- |
| | | | CAUGGUUAGU | | | | SpBE3 |
| P1829LVS/F | CCC | YYC | GAUCUGCCCA | 810 | (TGAC) | 20 (C7/8) | VQR- |
| | | | UGGUUAGUGG | | | | SpBE3 |
| P1829LVS/F | CCC | YYC | UGCCCAUGGU | 811 | (CGG) | 20 (C3/4) | SpBE3 |
| | | | UAGUGGUGAC | | | | |
| P1829LVS/F | CCC | YYC | GCCCAUGGUU | 812 | (GGAT) | 20 (C2/3) | VQR- |
| | | | AGUGGUGACC | | | | SpBE3 |
| P1829LVS/F | CCC | YYC | UUGCCAUGGA | 813 | (GTTAGT) | 20 (C15/16) | KKH- |
| | | | UCUGCCCAUG | | | | SaBE3 |
| P1829LVS/F | CCC | YYC | CCAUGGAUCU | 814 | (AGTGGT) | 20 (C12/13) | KKH- |
| | | | GCCCAUGGUU | | | | SaBE3 |
| P1829LVS/F | CCC | YYC | CUGCCCAUGG | 815 | (CCGGAT) | 20 (C4/5) | SaBE3 |
| | | | UUAGUGGUGA | | | | |
| P1829LVS/F | CCC | YYC | AUGGAUCUGC | 816 | (TGGTG) | 20 (C10/11) | St3BE3 |
| | | | CCAUGGUUAG | | | | |
| P1872LVS/F | CCT | YYT | AUGUCUGCAA | 817 | (AGTG) | 20 (C13/14) | VQR- |
| | | | AUCCUUCCAA | | | | SpBE3 |
| P1872LVS/F | CCT | YYT | AAUCCUUCCA | 818 | (TGAA) | 20 (C4/5) | VQR- |
| | | | AAGUGUCCUA | | | | SpBE3 |
| P1872LVS/F | CCT | YYT | UUCAUGUCUG | 819 | (CAAAGT) | 20 (C16/17) | KKH- |
| | | | CAAAUCCUUC | | | | SaBE3 |
| P1943LVS/F | CCA | YYA | GAACUCAAGU | 820 | (CAG) | 20 (C11/12) | SpBE3 |
| | | | CCAGAAAAAA | | | | |
| P1943LVS/F | CCA | YYA | AACUCAAGUC | 821 | (AGAT) | 20 (C10/11) | VQR- |
| | | | CAGAAAAAAC | | | | SpBE3 |
| P1943LVS/F | CCA | YYA | AGAACUCAAG | 822 | (ACAGAT) | 20 (C12/13) | KKH- |
| | | | UCCAGAAAAA | | | | SaBE3 |
| P1955LVS/F | CCA | YYA | CCACCACCUC | 823 | (TATGAT) | 20 (C12/13) | KKH- |
| | | | UCCACCUUCA | | | | SaBE3 |
| P1955/6L/S/ | CCA | YYA | ACCACCUCUC | 824 | (TGAT) | 20 (C10-14) | VQR- |
| F | | | CACCUUCAUA | | | | SpBE3 |
| P1955/6LVS/ | CCA | YYA | ACCUCUCCAC | 825 | (TAG) | 20 (C7-11) | SpBE3 |
| F | | | CUUCAUAUGA | | | | |
| P1955/6LVS/ | CCA | YYA | CCUCUCCACC | 826 | (AGTG) | 20 (C6-10) | VQR- |
| F | | | UUCAUAUGAU | | | | SpBE3 |
| P1955/6L/S/ | CCA | YYA | CCACCUCUCC | 827 | (GATAGT) | 20 (C9-13) | KKH- |
| F | | | ACCUUCAUAU | | | | SaBE3 |
| P1964LVS/F | CCA | YYA | AGUGUAACAA | 828 | (AGAG) | 20 (C13/14) | EQR- |
| | | | AGCCAGACAA | | | | SpBE3 |
| P1964LVS/F | CCA | YYA | GUGUAACAAA | 829 | (GAG) | 20 (C12/13) | SpBE3 |
| | | | GCCAGACAAA | | | | |
| P1964LVS/F | CCA | YYA | UGUAACAAAG | 830 | (AGAA) | 20 (C11/12) | VQR- |
| | | | CCAGACAAAG | | | | SpBE3 |
| P1964LVS/F | CCA | YYA | AAGCCAGACA | 831 | (TGAA) | 20 (C4/5) | VQR- |
| | | | AAGAGAAAUA | | | | SpBE3 |
| P1964LVS/F | CCA | YYA | UGUAACAAAG | 832 | (AGAAAT) | 20 (C11/12) | KKH- |
| | | | CCAGACAAAG | | | | SaBE3 |
| P1964LVS/F | CCA | YYA | AGUGUAACAA | 833 | (AGAGAAA) | 20 (C13/14) | St1BE3 |
| | | | AGCCAGACAA | | | | |

^{a}BE types: SpBE3 = APOBEC1-SpCas9n-UGI; VQR-SpBE3 = APOBEC1-VQR-SpCas9n-UGI: EQR-SpBE3 = APOBEC1-EQR-SpCas9n-UGI; VRER-SpBE3 = APOBEC1-VRER-SpCas9n-UGI; SaBE3 = APOBEC1-SaCas9n-UGI; KKH-SaBE3 = APOBEC1-KKH-SaCas9n-UGI; St3BE3 = APOBEC1-St3Cas9n-UGI; St1BE3 = APOBEC1-St1Cas9n-UGI.
^{b}Phospho-serine site S1490.

In some embodiments, editing of an ion channel-encoding nucleotide results in a destabilized or misfolded ion channel protein. An ion channel mutant comprising one or more destabilizing mutations described herein may have reduced activity compared to the wild type ion channel protein. For example, the activity of an ion channel variant comprising one or more destabilizing mutations described herein may be reduced by at least about 20%, at least about 30%, at least about 40%, at least about 50%, at least about 60%, at least about 70%, at least about 80%, at least about 90%, at least about 95%, at least about 99%, or more.

### Premature Stop Codons

Some aspects of the present disclosure provide strategies of editing an ion channel gene (e.g., SCN9A gene) to reduce the amount of full-length, functional ion channel protein (e.g., NaV1.7 protein) being produced. In some embodiments, stop codons may be introduced into the coding sequence of an ion channel gene upstream of the normal stop codon (referred to as a “premature stop codon”). Premature stop codons cause premature translation termination, in turn resulting in truncated and nonfunctional proteins and induces rapid degradation of the mRNA via the non-sense mediated mRNA decay pathway. See, e.g., Baker et al., Current Opinion in Cell Biology 16 (3): 293-299, 2004; Chang et al., Annual Review of Biochemistry 76: 51-74, 2007; and Behm-Ansmant et al., Genes & Development 20 (4): 391-398, 2006, each of which is incorporated herein by reference.

The nucleobase editors may be used to convert several amino acid codons to a stop codon (e.g., TAA, TAG, or TGA). For example, nucleobase editors including a cytosine deaminase domain are capable of converting a cytosine (C) base to a thymine (T) base via deamination. Thus, it is envisioned that, for amino acid codons containing a C base, the C base may be converted to T. For example, a CAG (Gln/Q) codon may be changed to a TAG (amber) codon via the deamination of the first C on the coding strand. For sense codons that contain a guanine (G) base, a C base is present on the complementary strand; and the G base may be converted to an adenosine (A) via the deamination of the C on the complementary strand. For example, a TGG (Trp/W) codon may be converted to a TAG (amber) codon via the deamination of the second C on the complementary strand. In some embodiments, two C to T changes are required to convert a codon to a nonsense codon. For example, a CGG (R) codon is converted to a TAG (amber) codon via the deamination of the first C on the coding strand and the deamination of the second C on the complementary strand. Non-limiting examples of codons that may be changed to stop codons via base editing are provided in Table 3.

TABLE 3
Conversion to Stop Codon

| Target codon | Base-editing process | Edited codon |
|---|---|---|
| CAG (Gln/Q) | 1^{st} base C to T on coding strand | TAG (amber) |
| TGG (Trp/W) | 2^{nd} base C to T on complementary strand | TAG (amber) |
| CGA (Arg/R) | 1^{st} base C to T on coding strand | TGA (opal) |
| CAA (Gln/Q) | 1^{st} base C to T on coding strand | TAA (ochre) |
| TGG (Trp/W) | 3^{rd} base C to T on complementary strand | TGA (opal) |
| CGG (Arg/R) | 1^{st} base C to T on coding strand and 2^{nd} base C to T | TAG (amber) |
| | on complementary strand | |
| CGA (Arg/R) | 1^{st} base C to T on coding strand and 2^{nd} base C to T | TAA (orchre) |
| | on complementary strand | |

* single underline: changes on the coding strand
double underline: changes on the complementary strand

Non-limiting examples of codons in the SCN9A gene that may be changed to stop codons by the nucleobase editor are provided in Table 4. In some embodiments, the introduction of stop codons may be efficacious in generating truncations when the target residue is located in a flexible loop. In some embodiments, two codons adjacent to each other may both be converted to stop codons, resulting in two stop codons adjacent to each other (also referred to as “tandem stop codons”). “Adjacent” means there are no more than 5 amino acids between the two stop codons. For example, the two stop codons may be immediately adjacent to each other (0 amino acids in between) or have 1, 2, 3, 4, or 5 amino acids in between. Non-limiting examples of tandem stop codons that may be introduced are listed in Table 4 (e.g., Q368X/Q369X, Q408X/Q410X. Q1539X/Q1541X, wherein X is a stop codon). In some embodiments, a stop codon is introduced adjacent to a structurally destabilizing mutation.

TABLE 4
Exemplary NaV1.7 (SNA9A) Protective Loss-of-Function Mutations via
Premature Stop Codons

| | | | | | | gRNA | |
|---|---|---|---|---|---|---|---|
| | | | | SEQ | | size | |
| Residue | Codon | Resulting | Programmable guide-RNA | ID | | (C | |
| Change | Change | Codon(s) | sequence | NOs | (PAM) | edited) | BE type^{a} |
| Q18X | CAG | TAG | AAACAGUCUCUUGCCCUCAU | 834 | (TGAA) | 20 (C4) | VQR- |
| | | | | | | | SpBE3 |
| Q25X | CAA | TAA | CUCAUUGAACAACGCAUUGC | 835 | (TGAA) | 20 | VQR- |
| | | | | | | (C10) | SpBE3 |
| Q25X | CAA | TAA | AUUGAACAACGCAUUGCUGA | 836 | (AAG) | 20 (C7) | SpBE3 |
| Q25X | CAA | TAA | UUGAACAACGCAUUGCUGAA | 837 | (AGAA) | 20 (C6) | VQR- |
| | | | | | | | SpBE3 |
| Q25X | CAA | TAA | UGAACAACGCAUUGCUGAAA | 838 | (GAAAAT) | 20 (C5) | KKH- |
| | | | | | | | SaBE3 |
| Q25X | CAA | TAA | CAUUGAACAACGCAUUGCUG | 839 | (AAAGAAA) | 20 (C8) | St1BE3 |
| Q58X | CAG | TAG | AAACAGCUGCCCUUCAUCUA | 840 | (TGG) | 20 (C4) | SpBE3 |
| Q58X | CAG | TAG | AACAGCUGCCCUUCAUCUAU | 841 | (GGG) | 20 (C3) | SpBE3 |
| Q58X | CAG | TAG | ACAGCUGCCCUUCAUCUAUG | 842 | (GGG) | 20 (C2) | SpBE3 |
| Q58X | CAG | TAG | CAGCUGCCCUUCAUCUAUGG | 843 | (GGAC) | 20(C1) | VQR- |
| | | | | | | | SpBE3 |
| Q58X | CAG | TAG | AAACAGCUGCCCUUCAUCUA | 844 | (TGGGG) | 20 (C4) | St3BE3 |
| W151X | TGG | TAR | CCAGUCCGGUGGGUUAUUCA | 845 | (TGG) | 20(C2) | SpBE3 |
| W151X | TGG | TAR | CAUUUUUGGUCCAGUCCGGU | 846 | (GGG) | 20 | SpBE3 |
| | | | | | | (C12) | |
| W151X | TGG | TAR | ACAUUUUUGGUCCAGUCCGG | 847 | (TGG) | 20 | SpBE3 |
| | | | | | | (C13) | |
| W151X | TGG | TAR | GUCCAGUCCGGUGGGUUAUU | 848 | (CATGGT) | 20(C4) | KKH- |
| | | | | | | | SaBE3 |
| W151X | TGG | TAR | GACAUUUUUGGUCCAGUCCG | 849 | (GTGGGT) | 20 | SaBE3 |
| | | | | | | (C14) | |
| W188X | TGG | TAR | CCACGGGUCACGAAGAAAAG | 850 | (TGAA) | 20 (C2) | VQR- |
| | | | | | | | SpBE3 |
| W188X | TGG | TAR | UUCCACGGGUCACGAAGAAA | 851 | (AGTG) | 20(C4) | VQR- |
| | | | | | | | SpBE3 |
| W188X | TGG | TAR | GUUCCACGGGUCACGAAGAA | 852 | (AAG) | 20 (C5) | SpBE3 |
| W188X | TGG | TAR | GCCAGUUCCACGGGUCACGA | 853 | (AGAA) | 20 | VQR- |
| | | | | | | (C9/3) | SpBE3 |
| W188X | TGG | TAR | AGCCAGUUCCACGGGUCACG | 854 | (AAG) | 20 | SpBE3 |
| | | | | | | (C10/4) | |
| W188X | TGG | TAR | CCAGCCAGUUCCACGGGUCA | 855 | (CGAA) | 20 | VQR- |
| | | | | | | (C12/6) | SpBE3 |
| W188X | TGG | TAR | UCCACGGGUCACGAAGAAAA | 856 | (GTGAAT) | 20 (C3) | SaBE3 |
| W188X | TGG | TAR | CAGUUCCACGGGUCACGAAG | 857 | (AAAAGT) | 20 | KKH- |
| | | | | | | (C7/1) | SaBE3 |
| W188X | TGG | TAR | CAGCCAGUUCCACGGGUCAC | 858 | (GAAGAAA) | 20 | St1BE3 |
| | | | | | | (C11/5) | |
| W190X | TGG | TAR | CAAAAUCCAGCCAGUUCCAC | 859 | (GGG) | 20 | SpBE3 |
| | | | | | | (C12) | |
| W190X | TGG | TAR | ACAAAAUCCAGCCAGUUCCA | 860 | (CGG) | 20 | SpBE3 |
| | | | | | | (C13) | |
| W190X | TGG | TAR | GACAAAAUCCAGCCAGUUCC | 861 | (ACGGGT) | 20 | SaBE3 |
| | | | | | | (C14) | |
| R214X | CGA | TGA | GUUUCAGCUCUUCGAACUUU | 862 | (CAG) | 20 | SpBE3 |
| | | | | | | (C13) | |
| R214X | CGA | TGA | UUUCAGCUCUUCGAACUUUC | 863 | (AGAG) | 20 | EQR- |
| | | | | | | (C12) | SpBE3 |
| R214X | CGA | TGA | UUCAGCUCUUCGAACUUUCA | 864 | (GAG) | 20 | SpBE3 |
| | | | | | | (C11) | |
| R214X | CGA | TGA | UCUUCGAACUUUCAGAGUAU | 865 | (TGAG) | 20(C5) | EQR- |
| | | | | | | | SpBE3 |
| R214X | CGA | TGA | CUUCGAACUUUCAGAGUAUU | 866 | (GAG) | 20 (C4) | SpBE3 |
| R214X | CGA | TGA | UUCGAACUUUCAGAGUAUUG | 867 | (AGAG) | 20 (C3) | EQR- |
| | | | | | | | SpBE3 |
| R214X | CGA | TGA | UCGAACUUUCAGAGUAUUGA | 868 | (GAG) | 20 (C2) | SpBE3 |
| R214X | CGA | TGA | GUUUCAGCUCUUCGAACUUU | 869 | (CAGAGT) | 20 | SaBE3 |
| | | | | | | (C13) | |
| Q240X | CAG | TAG | GGGCUUUGAUCCAGUCAGUG | 870 | (AAG) | 20 | SpBE3 |
| | | | | | | (C12) | |
| Q240X | CAG | TAG | GGCUUUGAUCCAGUCAGUGA | 871 | (AGAA) | 20 | VQR- |
| | | | | | | (C11) | SpBE3 |
| 0240X | CAG | TAG | CUUUGAUCCAGUCAGUGAAG | 872 | (AAG) | 20 (C9) | SpBE3 |
| Q240X | CAG | TAG | CAGUCAGUGAAGAAGCUUUC | 873 | (TGAT) | 20 (C1) | VQR- |
| | | | | | | | SpBE3 |
| Q240X | CAG | TAG | UCCAGUCAGUGAAGAAGCUU | 874 | (TCTGAT) | 20 (C3) | KKH- |
| | | | | | | | SaBE3 |
| Q240X | CAG | TAG | GGGGCUUUGAUCCAGUCAGU | 875 | (GAAGAAG) | 20 | St1BE3 |
| | | | | | | (C13) | |
| Q265X | CAG | TAG | AAUUGGACUACAGCUGUUCA | 876 | (TGG) | 20 | SpBE3 |
| | | | | | | (C11) | |
| Q265X | CAG | TAG | AUUGGACUACAGCUGUUCAU | 877 | (GGG) | 20 | SpBE3 |
| | | | | | | (C10) | |
| Q265X | CAG | TAG | UUGGACUACAGCUGUUCAUG | 878 | (GGAA) | 20 (C9) | VQR- |
| | | | | | | | SpBE3 |
| Q265X | CAG | TAG | ACAGCUGUUCAUGGGAAACC | 879 | (TGAA) | 20 (C2) | VQR- |
| | | | | | | | SpBE3 |
| Q265X | CAG | TAG | AGCUGUUCAUGGGAAACCUG | 880 | (AAG) | 20 (C-1) | SpBE3 |
| R277X | CGA | TGA | AAAUGUUUUCGAAAUUCACU | 881 | (TGAA) | 20 | VQR- |
| | | | | | | (C10) | SpBE3 |
| R277X | CGA | TGA | CGAAAUUCACUUGAAAAUAA | 882 | (TGAA) | 20 (C1) | VQR- |
| | | | | | | | SpBE3 |
| R277X | CGA | TGA | AAUGUUUUCGAAAUUCACUU | 883 | (GAAAAT) | 20 (C9) | KKH- |
| | | | | | | | SaBE3 |
| R277X | CGA | TGA | GUUUUCGAAAUUCACUUGAA | 884 | (AATAAT) | 20 (C6) | KKH- |
| | | | | | | | SaBE3 |
| Q323X | CAG | TAG | CUCGUGUGUAGUCAGUGUCC | 885 | (AGAG) | 20 | EQR- |
| | | | | | | (C13) | SpBE3 |
| Q323X | CAG | TAG | UCGUGUGUAGUCAGUGUCCA | 886 | (GAG) | 20 | SpBE3 |
| | | | | | | (C12) | |
| Q323X | CAG | TAG | CGUGUGUAGUCAGUGUCCAG | 887 | (AGG) | 20 | SpBE3 |
| | | | | | | (C11) | |
| Q323X | CAG | TAG | GUGUGUAGUCAGUGUCCAGA | 888 | (GGG) | 20 | SpBE3 |
| | | | | | | (C10) | |
| Q323X | CAG | TAG | UGUGUAGUCAGUGUCCAGAG | 889 | (GGG) | 20 (C9) | SpBE3 |
| Q323X | CAG | TAG | AGUGUCCAGAGGGGUACACC | 890 | (TGTG) | 20 (C-1) | VQR- |
| | | | | | | | SpBE3 |
| Q323X | CAG | TAG | CGUGUGUAGUCAGUGUCCAG | 891 | (AGGGGT) | 20 | SaBE3 |
| | | | | | | (C11) | |
| Q323X | CAG | TAG | CGUGUGUAGUCAGUGUCCAG | 892 | (AGGGG) | 20 | St3BE3 |
| | | | | | | (C11) | |
| W349X | TGG | TAR | CAGCUGAAAGUGUCAAAGCU | 893 | (CGTG) | 20 (C1) | VQR- |
| | | | | | | | SpBE3 |
| W349X | TGG | TAR | AGGCCCAGCUGAAAGUGUCA | 894 | (AAG) | 20 (C6) | SpBE3 |
| W349X | TGG | TAR | GCUAAGAAGGCCCAGCUGAA | 895 | (AGTG) | 20 | VQR- |
| | | | | | | (C13) | SpBE3 |
| W349X | TGG | TAR | AAGGCUAAGAAGGCCCAGCU | 896 | (GAAAGT) | 20 | KKH- |
| | | | | | | (C16) | SaBE3 |
| Q360X | CAA | TAA | GGCUAAUGACCCAAGAUUAC | 897 | (TGG) | 20 | SpBE3 |
| | | | | | | (C12) | |
| Q360X | CAA | TAA | GCUAAUGACCCAAGAUUACU | 898 | (GGG) | 20 | SpBE3 |
| | | | | | | (C11) | |
| Q360X | CAA | TAA | CUAAUGACCCAAGAUUACUG | 899 | (GGAA) | 20 | VQR- |
| | | | | | | (C10) | SpBE3 |
| W363X | TGG | TAR | UCCCAGUAAUCUUGGGUCAU | 900 | (TAG) | 20 (C4) | SpBE3 |
| W363X | TGG | TAR | AAGGUUUUCCCAGUAAUCUU | 901 | (GGG) | 20 | SpBE3 |
| | | | | | | (C11) | |
| W363X | TGG | TAR | AAAGGUUUUCCCAGUAAUCU | 902 | (TGG) | 20 | SpBE3 |
| | | | | | | (C12) | |
| W363X | TGG | TAR | UAAAGGUUUUCCCAGUAAUC | 903 | (TTGGGT) | 20 | SaBE3 |
| | | | | | | (C13) | |
| Q368/9X | CAA | TAA | UUUACCAACAGGUGAGUACC | 904 | (AAG) | 20 (C6) | SpBE3 |
| Q368/9X | CAA | TAA | UUACCAACAGGUGAGUACCA | 905 | (AGAG) | 20 (C5) | EQR- |
| | | | | | | | SpBE3 |
| Q368/9X | CAA | TAA | UACCAACAGGUGAGUACCAA | 906 | (GAG) | 20 (C4) | SpBE3 |
| Q368/9X | CAA | TAA | ACCAACAGGUGAGUACCAAG | 907 | (AGAA) | 20 (C3) | VQR- |
| | | | | | | | SpBE3 |
| Q368/9X | CAA | TAA | UUACCAACAGGUGAGUACCA | 908 | (AGAGAAA) | 20 (C5) | St1BE3 |
| Q369X | CAG | TAG | UUUACCAACAGGUGAGUACC | 909 | (AAG) | 20 (C9) | SpBE3 |
| Q369X | CAG | TAG | UUACCAACAGGUGAGUACCA | 910 | (AGAG) | 20 (C8) | EQR- |
| | | | | | | | SpBE3 |
| Q369X | CAG | TAG | UACCAACAGGUGAGUACCAA | 911 | (GAG) | 20 (C7) | SpBE3 |
| Q369X | CAG | TAG | ACCAACAGGUGAGUACCAAG | 912 | (AGAA) | 20 (C6) | VQR- |
| | | | | | | | SpBE3 |
| Q369X | CAG | TAG | UUACCAACAGGUGAGUACCA | 913 | (AGAGAAA) | 20 (C8) | St1BE3 |
| Q408/10X | CAG | TAG | GAACAGAACCAGGCAAACAU | 914 | (TGAA) | 20 | VQR- |
| | | | | | | (C4/10) | SpBE3 |
| Q408/10X | CAG | TAG | ACAGAACCAGGCAAACAUUG | 915 | (AAG) | 20 | SpBE3 |
| | | | | | | (C2/8) | |
| Q408/10X | CAG | TAG | CAGAACCAGGCAAACAUUGA | 916 | (AGAA) | 20 | VQR- |
| | | | | | | (C1/7) | SpBE3 |
| Q408/10X | CAG | TAG | AACAGAACCAGGCAAACAUU | 917 | (GAAGAAG) | 20 | St1BE3 |
| | | | | | | (C3/9) | |
| Q410X | CAG | TAG | GAACCAGGCAAACAUUGAAG | 918 | (AAG) | 20 (C5) | SpBE3 |
| Q418X | CAG | TAG | AGAAGCUAAACAGAAAGAAU | 919 | (TAG) | 20 | SpBE3 |
| | | | | | | (C11) | |
| Q418X | CAG | TAG | GAAGCUAAACAGAAAGAAUU | 920 | (AGAA) | 20 | VQR- |
| | | | | | | (C10) | SpBE3 |
| Q418X | CAG | TAG | AGAAAGAAUUAGAAUUUCAA | 921 | (CAG) | 20 (C-1) | SpBE3 |
| Q418X | CAG | TAG | AGAAGCUAAACAGAAAGAAU | 922 | (TAGAAT) | 20 | SaBE3 |
| | | | | | | (C11) | |
| Q418X | CAG | TAG | CAGAAAGAAUUAGAAUUUCA | 923 | (ACAGAT) | 20 (C1) | KKH- |
| | | | | | | | SaBE3 |
| Q418X | CAG | TAG | AAGAAGCUAAACAGAAAGAA | 924 | (TTAGAAT) | 20 | St1BE3 |
| | | | | | | (C12) | |
| Q424X | CAA | TAA | AUUAGAAUUUCAACAGAUGU | 925 | (TAG) | 20 | SpBE3 |
| | | | | | | (C11) | |
| Q424X | CAA | TAA | UUAGAAUUUCAACAGAUGUU | 926 | (AGAC) | 20 | VQR- |
| | | | | | | (C10) | SpBE3 |
| Q425X | CAG | TAG | UUAGAAUUUCAACAGAUGUU | 927 | (AGAC) | 20 | VQR- |
| | | | | | | (C13) | SpBE3 |
| Q434X | CAA | TAA | AAAAAAGAGCAAGAAGAAGC | 928 | (TGAG) | 20 | EQR- |
| | | | | | | (C10) | SpBE3 |
| Q434X | CAA | TAA | AAAAAGAGCAAGAAGAAGCU | 929 | (GAG) | 20 (C9) | SpBE3 |
| Q434X | CAA | TAA | AAAAGAGCAAGAAGAAGCUG | 930 | (AGG) | 20 (C8) | SpBE3 |
| Q434X | CAA | TAA | AAAAAAGAGCAAGAAGAAGC | 931 | (TGAGGT) | 20 | KKH- |
| | | | | | | (C10) | SaBE3 |
| Q485X | CAA | TAA | AAGAAUCAAAAGAAGCUCUC | 932 | (CAG) | 20 (C7) | SpBE3 |
| Q485X | CAA | TAA | AGAAUCAAAAGAAGCUCUCC | 933 | (AGTG) | 20 (C6) | VQR- |
| | | | | | | | SpBE3 |
| Q485X | CAA | TAA | AAUCAAAAGAAGCUCUCCAG | 934 | (TGG) | 20 (C4) | SpBE3 |
| Q485X | CAA | TAA | AUCAAAAGAAGCUCUCCAGU | 935 | (GGAG) | 20 (C3) | EQR- |
| | | | | | | | SpBE3 |
| Q485X | CAA | TAA | UCAAAAGAAGCUCUCCAGUG | 936 | (GAG) | 20 (C2) | SpBE3 |
| Q485X | CAA | TAA | CAAAAGAAGCUCUCCAGUGG | 937 | (AGAG) | 20 (C1) | EQR- |
| | | | | | | | SpBE3 |
| Q485X | CAA | TAA | AAAAGAAGCUCUCCAGUGGA | 938 | (GAG) | 20 (C-1) | SpBE3 |
| Q485X | CAA | TAA | AAAAGAAUCAAAAGAAGCUC | 939 | (TCCAGT) | 20 (C9) | KKH- |
| | | | | | | | SaBE3 |
| Q485X | CAA | TAA | AAUCAAAAGAAGCUCUCCAG | 940 | (TGGAG) | 20 (C4) | St3BE3 |
| R523X | CGA | TGA | GAAGGGCAUAGGCGAGCACA | 941 | (TGAA) | 20 | VQR- |
| | | | | | | (C13) | SpBE3 |
| R523X | CGA | TGA | GGCAUAGGCGAGCACAUGAA | 942 | (AAG) | 20 (C9) | SpBE3 |
| R523X | CGA | TGA | GCAUAGGCGAGCACAUGAAA | 943 | (AGAG) | 20 (C8) | EQR- |
| | | | | | | | SpBE3 |
| R523X | CGA | TGA | CAUAGGCGAGCACAUGAAAA | 944 | (GAG) | 20 (C7) | SpBE3 |
| R523X | CGA | TGA | AUAGGCGAGCACAUGAAAAG | 945 | (AGG) | 20 (C6) | SpBE3 |
| R523X | CGA | TGA | GCAUAGGCGAGCACAUGAAA | 946 | (AGAGGT) | 20 (C8) | KKH- |
| | | | | | | | SaBE3 |
| Q534X | CAG | TAG | UACCCCCAAUCAGGUACCAC | 947 | (CCAAAT) | 20 | KKH- |
| | | | | | | (C11) | SaBE3 |
| Q534X | CAG | TAG | AUCAGGUACCACCCAAAUUG | 948 | (CTAAAT) | 20 (C3) | KKH- |
| | | | | | | | SaBE3 |
| R548X | CGA | TGA | UUUUCUGCAAGGCGAAGCAG | 949 | (CAG) | 20 | SpBE3 |
| | | | | | | (C13) | |
| R548X | CGA | TGA | UUUCUGCAAGGCGAAGCAGC | 950 | (AGAA) | 20 | VQR- |
| | | | | | | (C12) | SpBE3 |
| R548X | CGA | TGA | GCAAGGCGAAGCAGCAGAAC | 951 | (AAG) | 20 (C7) | SpBE3 |
| R548X | CGA | TGA | CUGCAAGGCGAAGCAGCAGA | 952 | (ACAAGT) | 20 (C9) | KKH- |
| | | | | | | | SaBE3 |
| R548X | CGA | TGA | GAAGCAGCAGAACAAGUCUU | 953 | (TTTAGT) | 20 (C-1) | KKH- |
| | | | | | | | SaBE3 |
| Q595X | CAG | TAG | GUUUGUGCCCCACAGACCCC | 954 | (AGG) | 20 | SpBE3 |
| | | | | | | (C13) | |
| Q595X | CAG | TAG | UUUGUGCCCCACAGACCCCA | 955 | (GGAG) | 20 | EQR- |
| | | | | | | (C12) | SpBE3 |
| Q595X | CAG | TAG | UUGUGCCCCACAGACCCCAG | 956 | (GAG) | 20 | SpBE3 |
| | | | | | | (C11) | |
| Q595X | CAG | TAG | UGUGCCCCACAGACCCCAGG | 957 | (AGCG) | 20 | VRER- |
| | | | | | | (C10) | SpBE3 |
| Q595X | CAG | TAG | UGCCCCACAGACCCCAGGAG | 958 | (CGAC) | 20 (C8) | VQR- |
| | | | | | | | SpBE3 |
| Q595X | CAG | TAG | CACAGACCCCAGGAGCGACG | 959 | (CAG) | 20 (C3) | SpBE3 |
| Q595X | CAG | TAG | AGACCCCAGGAGCGACGCAG | 960 | (CAG) | 20 (C-1) | SpBE3 |
| Q595X | CAG | TAG | ACAGACCCCAGGAGCGACGC | 961 | (AGCAGT) | 20 (C2) | KKH- |
| | | | | | | | SaBE3 |
| Q595X | CAG | TAG | GUUUGUGCCCCACAGACCCC | 962 | (AGGAG) | 20 | St3BE3 |
| | | | | | | (C13) | |
| R597X | CGA | TGA | AGACCCCAGGAGCGACGCAG | 963 | (CAG) | 20 | SpBE3 |
| | | | | | | (C13) | |
| R597X | CGA | TGA | GAGCGACGCAGCAGUAACAU | 964 | (CAG) | 20 (C4) | SpBE3 |
| Q604X | CAA | TAA | AGUAACAUCAGCCAAGCCAG | 965 | (TAG) | 20 | SpBE3 |
| | | | | | | (C13) | |
| Q604X | CAA | TAA | GUAACAUCAGCCAAGCCAGU | 966 | (AGG) | 20 | SpBE3 |
| | | | | | | (C12) | |
| Q604X | CAA | TAA | CAGUAACAUCAGCCAAGCCA | 967 | (GTAGGT) | 20 | KKH- |
| | | | | | | (C14) | SaBE3 |
| Q604X | CAA | TAA | AGCCAAGCCAGUAGGUCCCC | 968 | (ACCAAT) | 20 (C4) | KKH- |
| | | | | | | | SaBE3 |
| Q643X | CAG | TAG | CCCCAAUGGACAGCUUCUGC | 969 | (CAG) | 20 | SpBE3 |
| | | | | | | (C11) | |
| Q643X | CAG | TAG | CCCAAUGGACAGCUUCUGCC | 970 | (AGAG) | 20 | EQR- |
| | | | | | | (C10) | SpBE3 |
| Q643X | CAG | TAG | CCAAUGGACAGCUUCUGCCA | 971 | (GAG) | 20 (C9) | SpBE3 |
| Q643X | CAG | TAG | CAAUGGACAGCUUCUGCCAG | 972 | (AGG) | 20 (C8) | SpBE3 |
| Q643X | CAG | TAG | AAUGGACAGCUUCUGCCAGA | 973 | (GGTG) | 20 (C7) | VQR- |
| | | | | | | | SpBE3 |
| Q643X | CAG | TAG | UGGACAGCUUCUGCCAGAGG | 974 | (TGAT) | 20 (C5) | VQR- |
| | | | | | | | SpBE3 |
| Q643X | CAG | TAG | CCCAAUGGACAGCUUCUGCC | 975 | (AGAGGT) | 20 | KKH- |
| | | | | | | (C10) | SaBE3 |
| Q643X | CAG | TAG | AAUGGACAGCUUCUGCCAGA | 976 | (GGTGAT) | 20 (C7) | KKH- |
| | | | | | | | SaBE3 |
| Q643X | CAG | TAG | GGACAGCUUCUGCCAGAGGU | 977 | (GATAAT) | 20 (C4) | KKH- |
| | | | | | | | SaBE3 |
| Q643X | CAG | TAG | AGCUUCUGCCAGAGGUGAUA | 978 | (ATAGAT) | 20 (C-1) | KKH- |
| | | | | | | | SaBE3 |
| Q643X | CAG | TAG | CAAUGGACAGCUUCUGCCAG | 979 | (AGGTG) | 20 (C8) | St3BE3 |
| Q663X | CAA | TAA | GCACGACCAAUCAAAUACAC | 980 | (AAG) | 20 (C8) | SpBE3 |
| Q663X | CAA | TAA | CACGACCAAUCAAAUACACA | 981 | (AGAA) | 20 (C7) | VQR- |
| | | | | | | | SpBE3 |
| Q663X | CAA | TAA | ACCAAUCAAAUACACAAGAA | 982 | (AAG) | 20 (C3) | SpBE3 |
| Q663X | CAA | TAA | CCAAUCAAAUACACAAGAAA | 983 | (AGG) | 20 (C2) | SpBE3 |
| Q663X | CAA | TAA | CAAUCAAAUACACAAGAAAA | 984 | (GGCG) | 20 (C1) | VRER- |
| | | | | | | | SpBE3 |
| Q663X | CAA | TAA | GGCACGACCAAUCAAAUACA | 985 | (CAAGAAA) | 20 (C9) | St1BE3 |
| Q663X | CAA | TAA | CCAAUCAAAUACACAAGAAA | 986 | (AGGCG) | 20 (C2) | St3BE3 |
| Q687X | CAG | TAG | CAACCUCAGACAGAGAGCAA | 987 | (TGAG) | 20 (C7) | EQR- |
| | | | | | | | SpBE3 |
| Q687X | CAG | TAG | AACCUCAGACAGAGAGCAAU | 988 | (GAG) | 20 (C6) | SpBE3 |
| Q687X | CAG | TAG | CUCAGACAGAGAGCAAUGAG | 989 | (TAG) | 20 (C3) | SpBE3 |
| Q687X | CAG | TAG | UCAGACAGAGAGCAAUGAGU | 990 | (AGAG) | 20 (C5) | EQR- |
| | | | | | | | SpBE3 |
| Q687X | CAG | TAG | CAGACAGAGAGCAAUGAGUA | 991 | (GAG) | 20 (C1) | SpBE3 |
| Q687X | CAG | TAG | GAUCCCAACCUCAGACAGAG | 992 | (AGCAAT) | 20 | KKH- |
| | | | | | | (C12) | SaBE3 |
| Q687X | CAG | TAG | CCAACCUCAGACAGAGAGCA | 993 | (ATGAGT) | 20 (C8) | SaBE3 |
| Q708X | CAA | TAA | CCAGACAAAAAUGUCCACCU | 994 | (TGG) | 20 (C6) | SpBE3 |
| Q708X | CAA | TAA | CAGACAAAAAUGUCCACCUU | 995 | (GGTG) | 20 (C5) | VQR- |
| | | | | | | | SpBE3 |
| Q708X | CAA | TAA | GACAAAAAUGUCCACCUUGG | 996 | (TGG) | 20 (C3) | SpBE3 |
| | | | | | | 20 (C8) | KKH- |
| Q708X | CAA | TAA | GUCCAGACAAAAAUGUCCAC | 997 | (CTTGGT) | | SaBE3 |
| Q708X | CAA | TAA | CAGACAAAAAUGUCCACCUU | 998 | (GGTGGT) | 20 (C5) | KKH- |
| | | | | | | | SaBE3 |
| Q708X | CAA | TAA | CCAGACAAAAAUGUCCACCU | 999 | (TGGTG) | 20 (C6) | St3BE3 |
| W713X | TGG | TAR | CAAGGUGGACAUUUUUGUCU | 1000 | (GGAC) | 20(C1) | VQR- |
| | | | | | | | SpBE3 |
| W713X | TGG | TAR | CCAAGGUGGACAUUUUUGUC | 1001 | (TGG) | 20(C2) | SpBE3 |
| W714X | TGG | TAR | CAAAUCUGUACCACCAAGGU | 1002 | (GGAC) | 20 | VQR- |
| | | | | | | (C12) | SpBE3 |
| W714X | TGG | TAR | GCAAAUCUGUACCACCAAGG | 1003 | (TGG) | 20 | SpBE3 |
| | | | | | | (C13) | |
| W724X | TGG | TAR | AAUUCCAGAUCAAGAAUUUG | 1004 | (TGTG) | 20 (C6) | VQR- |
| | | | | | | | SpBE3 |
| W724X | TGG | TAR | GCAAUUCCAGAUCAAGAAUU | 1005 | (TGTG) | 20 (C8) | VQR- |
| | | | | | | | SpBE3 |
| W724X | TGG | TAR | UCCAGAUCAAGAAUUUGUGU | 1006 | (GCAAAT) | 20 (C3) | KKH- |
| | | | | | | | SaBE3 |
| W724X | TGG | TAR | UAUGGAGAGCAAUUCCAGAU | 1007 | (CAAGAAT) | 20 | St1BE3 |
| | | | | | | (C16) | |
| W730X | TGG | TAR | CCAAUAUGGAGAGCAAUUCC | 1008 | (AGAT) | 20(C2) | VQR- |
| | | | | | | | SpBE3 |
| W730X | TGG | TAR | UCCAAUAUGGAGAGCAAUUC | 1009 | (CAG) | 20 (C3) | SpBE3 |
| W730X | TGG | TAR | UGAAUUUUAUCCAAUAUGGA | 1010 | (GAG) | 20 | SpBE3 |
| | | | | | | (C12) | |
| W730X | TGG | TAR | UUGAAUUUUAUCCAAUAUGG | 1011 | (AGAG) | 20 | EQR- |
| | | | | | | (C13) | SpBE3 |
| W730X | TGG | TAR | AUCCAAUAUGGAGAGCAAUU | 1012 | (CCAGAT) | 20(C4) | KKH- |
| | | | | | | | SaBE3 |
| W730X | TGG | TAR | GAAUUUUAUCCAAUAUGGAG | 1013 | (AGCAAT) | 20 | KKH- |
| | | | | | | (C11) | SaBE3 |
| Q805X | CAA | TAA | AUGAGUAUUUCCAAGUAGGC | 1014 | (TGG) | 20 | SpBE3 |
| | | | | | | (C12) | |
| Q805X | CAA | TAA | UGAGUAUUUCCAAGUAGGCU | 1015 | (GGAA) | 20 | VQR- |
| | | | | | | (C11) | SpBE3 |
| Q805X | CAA | TAA | CAAGUAGGCUGGAAUAUUUU | 1016 | (TGAC) | 20 (C1) | VQR- |
| | | | | | | | SpBE3 |
| Q805X | CAA | TAA | AUGAGUAUUUCCAAGUAGGC | 1017 | (TGGAAT) | 20 | SaBE3 |
| | | | | | | (C12) | |
| W808X | TGG | TAR | AAAAAUAUUCCAGCCUACUU | 1018 | (GGAA) | 20 | VQR- |
| | | | | | | (C11) | SpBE3 |
| W808X | TGG | TAR | CAAAAAUAUUCCAGCCUACU | 1019 | (TGG) | 20 | SpBE3 |
| | | | | | | (C12) | |
| W808X | TGG | TAR | AAAAAUAUUCCAGCCUACUU | 1020 | (GGAAAT) | 20 | KKH- |
| | | | | | | (C11) | SaBE3 |
| R835X | CGA | TGA | UUGUCAGUUCUGCGAUCAUU | 1021 | (CAG) | 20 | SpBE3 |
| | | | | | | (C13) | |
| R835X | CGA | TGA | UGUCAGUUCUGCGAUCAUUC | 1022 | (AGAC) | 20 | VQR- |
| | | | | | | (C12) | SpBE3 |
| R835X | CGA | TGA | AGUUCUGCGAUCAUUCAGAC | 1023 | (TGG) | 20(C8) | SpBE3 |
| R835X | CGA | TGA | UCAGUUCUGCGAUCAUUCAG | 1024 | (ACTGGT) | 20 | KKH- |
| | | | | | | (C10) | SaBE3 |
| R841X | CGA | TGA | GCUUUUAGCUCCGAGUCUUC | 1025 | (AAG) | 20 | SpBE3 |
| | | | | | | (C12) | |
| R841X | CGA | TGA | UUAGCUCCGAGUCUUCAAGU | 1026 | (TGG) | 20 (C8) | SpBE3 |
| R841X | CGA | TGA | GCUCCGAGUCUUCAAGUUGG | 1027 | (CAAAAT) | 20(C5) | KKH- |
| | | | | | | | SaBE3 |
| W849X | TGG | TAR | CCAGGAUUUUGCCAACUUGA | 1028 | (AGAC) | 20(C2) | VQR- |
| | | | | | | | SpBE3 |
| W849X | TGG | TAR | GCCAGGAUUUUGCCAACUUG | 1029 | (AAG) | 20 (C3) | SpBE3 |
| W849X | TGG | TAR | UGGCCAGGAUUUUGCCAACU | 1030 | (TGAA) | 20 (C5) | VQR- |
| | | | | | | | SpBE3 |
| Q886X | CAG | TAG | GUGGUCGGCAUGCAGCUCUU | 1031 | (TGG) | 20 | SpBE3 |
| | | | | | | (C13) | |
| Q886X | CAG | TAG | UCGGCAUGCAGCUCUUUGGU | 1032 | (AAG) | 20 (C9) | SpBE3 |
| Q886X | CAG | TAG | CGGCAUGCAGCUCUUUGGUA | 1033 | (AGAG) | 20(C8) | EQR- |
| | | | | | | | SpBE3 |
| Q886X | CAG | TAG | GGCAUGCAGCUCUUUGGUAA | 1034 | (GAG) | 20 (C7) | SpBE3 |
| Q886X | CAG | TAG | AGCUCUUUGGUAAGAGCUAC | 1035 | (AAAGAAT) | 20 (C-1) | St1BE3 |
| W908X | TGG | TAR | UGUGCCACCGUGGGAGCGUA | 1036 | (CAG) | 20(C6) | SpBE3 |
| W908X | TGG | TAR | CGUUCAUGUGCCACCGUGGG | 1037 | (AGCG) | 20 | VRER- |
| | | | | | | (C12) | SpBE3 |
| W908X | TGG | TAR | UCGUUCAUGUGCCACCGUGG | 1038 | (GAG) | 20 | SpBE3 |
| | | | | | | (C13) | |
| W908X | TGG | TAR | CAUGUGCCACCGUGGGAGCG | 1039 | (TACAGT) | 20(C8) | KKH- |
| | | | | | | | SaBE3 |
| W908X | TGG | TAR | AGUCGUUCAUGUGCCACCGU | 1040 | (GGGAG) | 20 | St3BE3 |
| | | | | | | (C15) | |
| W928X | TGG | TAR | CCACUCUCCACACAGCACGC | 1041 | (GGAA) | 20 (C2) | VQR- |
| | | | | | | | SpBE3 |
| W928X | TGG | TAR | UCCACUCUCCACACAGCACG | 1042 | (CGG) | 20 (C3) | SpBE3 |
| W928X | TGG | TAR | UAUCCACUCUCCACACAGCA | 1043 | (CGCG) | 20(C5) | VRER- |
| | | | | | | | SpBE3 |
| W928X | TGG | TAR | GUCUCUAUCCACUCUCCACA | 1044 | (CAG) | 20 | SpBE3 |
| | | | | | | (C10) | |
| Q941X | CAA | TAA | GGAGGUCGCUGGUCAAGCUA | 1045 | (TGTG) | 20 | VQR- |
| | | | | | | (C14) | SpBE3 |
| Q989X | CAG | TAG | GCAAACAACCUCCAGAUUGC | 1046 | (AGTG) | 20 | VQR- |
| | | | | | | (C13) | SpBE3 |
| Q989X | CAG | TAG | AAACAACCUCCAGAUUGCAG | 1047 | (TGAC) | 20 | VQR- |
| | | | | | | (C11) | SpBE3 |
| Q989X | CAG | TAG | AACCUCCAGAUUGCAGUGAC | 1048 | (TAG) | 20 (C7) | SpBE3 |
| Q989X | CAG | TAG | ACCUCCAGAUUGCAGUGACU | 1049 | (AGAA) | 20 (C6) | VQR- |
| | | | | | | | SpBE3 |
| Q989X | CAG | TAG | AACCUCCAGAUUGCAGUGAC | 1050 | (TAGAAT) | 20 (C7) | SaBE3 |
| Q989X | CAG | TAG | CAACCUCCAGAUUGCAGUGA | 1051 | (CTAGAAT) | 20 (C8) | St1BE3 |
| Q1004X | CAA | TAA | AUUAUGUGAAACAAACCUUA | 1052 | (CGTG) | 20 | VQR- |
| | | | | | | (C12) | SpBE3 |
| Q1004X | CAA | TAA | UAUGUGAAACAAACCUUACG | 1053 | (TGAA) | 20 | VQR- |
| | | | | | | (C10) | SpBE3 |
| Q1004X | CAA | TAA | UUAUGUGAAACAAACCUUAC | 1054 | (GTGAAT) | 20 | SaBE3 |
| | | | | | | (C11) | |
| Q1026X | CAA | TAA | CAGGGAGAUAAGACAAGCAG | 1055 | (AAG) | 20 | SpBE3 |
| | | | | | | (C14) | |
| Q1026X | CAA | TAA | AGGGAGAUAAGACAAGCAGA | 1056 | (AGAT) | 20 | VQR- |
| | | | | | | (C13) | SpBE3 |
| Q1026X | CAA | TAA | GAUAAGACAAGCAGAAGAUC | 1057 | (TGAA) | 20 (C8) | VQR- |
| | | | | | | | SpBE3 |
| Q1026X | CAA | TAA | AAGCAGAAGAUCUGAAUACU | 1058 | (AAG) | 20 (C-1) | SpBE3 |
| Q1026X | CAA | TAA | AGAUAAGACAAGCAGAAGAU | 1059 | (CTGAAT) | 20 (C9) | SaBE3 |
| Q1077X | CAA | TAA | GUGAUGGUCAAUCAUUUAUU | 1060 | (CACAAT) | 20 (C9) | KKH- |
| | | | | | | | SaBE3 |
| W1161X | TGG | TAR | CAUACACAACCUGACAAGAA | 1061 | (AGAC) | 20 (C1) | VQR- |
| | | | | | | | SpBE3 |
| W1161X | TGG | TAR | CCAUACACAACCUGACAAGA | 1062 | (AAG) | 20(C2) | SpBE3 |
| W1161X | TGG | TAR | CCUCCAUACACAACCUGACA | 1063 | (AGAA) | 20(C5) | VQR- |
| | | | | | | | SpBE3 |
| W1161X | TGG | TAR | ACCUCCAUACACAACCUGAC | 1064 | (AAG) | 20(C6) | SpBE3 |
| W1161X | TGG | TAR | GAGAACCUCCAUACACAACC | 1065 | (TGAC) | 20 | VQR- |
| | | | | | | (C10) | SpBE3 |
| W1161X | TGG | TAR | AACCUCCAUACACAACCUGA | 1066 | (CAAGAAA) | 20(C7) | St1BE3 |
| Q1167X | CAA | TAA | CUCAUGCUGCCAAGUUAACA | 1067 | (TAG) | 20 | SpBE3 |
| | | | | | | (C11) | |
| Q1167X | CAA | TAA | CUCAUGCUGCCAAGUUAACA | 1068 | (TAGAGT) | 20 | SaBE3 |
| | | | | | | (C11) | |
| Q1167X | CAA | TAA | UCAUGCUGCCAAGUUAACAU | 1069 | (AGAG) | 20 | EQR- |
| | | | | | | (C10) | SpBE3 |
| Q1167X | CAA | TAA | CAUGCUGCCAAGUUAACAUA | 1070 | (GAG) | 20 (C9) | SpBE3 |
| Q1167X | CAA | TAA | CUGCCAAGUUAACAUAGAGU | 1071 | (CAG) | 20 (C5) | SpBE3 |
| Q1167X | CAA | TAA | UGCCAAGUUAACAUAGAGUC | 1072 | (AGG) | 20 (C4) | SpBE3 |
| Q1167X | CAA | TAA | GCCAAGUUAACAUAGAGUCA | 1073 | (GGG) | 20 (C3) | SpBE3 |
| Q1167X | CAA | TAA | CCAAGUUAACAUAGAGUCAG | 1074 | (GGAA) | 20 (C2) | VQR- |
| | | | | | | | SpBE3 |
| W1178/9X | TGG | TAR | CACCAGAUUUUUCCUUUCCC | 1075 | (TGAC) | 20 | VQR- |
| | | | | | | (C4/1) | SpBE3 |
| W1193X | TGG | TAR | AACUGUGUUCAACAAUCUUG | 1076 | (TAG) | 20(C-1) | SpBE3 |
| W1193X | TGG | TAR | CUUUCAAACCAACUGUGUUC | 1077 | (AACAAT) | 20 | KKH- |
| | | | | | | (C10) | SaBE3 |
| W1245X | TGG | TAR | UCCAUUUUAGAAGCAUUUCC | 1078 | (AGAA) | 20(C3) | VQR- |
| | | | | | | | SpBE3 |
| W1245X | TGG | TAR | AUCCAUUUUAGAAGCAUUUC | 1079 | (CAG) | 20(C4) | SpBE3 |
| W1245X | TGG | TAR | CCAUAUGCUAUCCAUUUUAG | 1080 | (AAG) | 20 | SpBE3 |
| | | | | | | (C13) | |
| W1245X | TGG | TAR | AUCCAUUUUAGAAGCAUUUC | 1081 | (CAGAAT) | 20 (C4) | SaBE3 |
| W1245X | TGG | TAR | UAUCCAUUUUAGAAGCAUUU | 1082 | (CCAGAAT) | 20 (C5) | St1BE3 |
| W1245X | TGG | TAR | AUAACCAUAUGCUAUCCAUU | 1083 | (TTAGAAG) | 20 | St1BE3 |
| | | | | | | (C17) | |
| W1245X | TGG | TAR | CAGCCAACACCAGGCAUUGG | 1084 | (TGAA) | 20 | VQR- |
| | | | | | | (C9/3) | SpBE3 |
| W1245X | TGG | TAR | UCCAGCCAACACCAGGCAUU | 1085 | (GGTG) | 20 | VQR- |
| | | | | | | (C11/5) | SpBE3 |
| W1245X | TGG | TAR | CAGCCAACACCAGGCAUUGG | 1086 | (TGAAAT) | 20 | KKH- |
| | | | | | | (C9/3) | SaBE3 |
| W1245X | TGG | TAR | AAAUCCAGCCAACACCAGGC | 1087 | (ATTGGT) | 20 | KKH- |
| | | | | | | (C14/8) | SaBE3 |
| W1245X | TGG | TAR | AUCCAGCCAACACCAGGCAU | 1088 | (TGGTG) | 20 | St3BE3 |
| | | | | | | (C12/6) | |
| W1245X | TGG | TAR | AUCCAGCCAACACCAGGCAU | 1089 | (TGG) | 20 | SpBE3 |
| | | | | | | (C12/6) | |
| W1332X | TGG | TAR | CAGAAUAUAAGACACACAAG | 1090 | (TAG) | 20 (C1) | SpBE3 |
| W1332X | TGG | TAR | AGCCAGAAUAUAAGACACAC | 1091 | (AAG) | 20 (C4) | SpBE3 |
| W1332X | TGG | TAR | UGAAUAUCAGCCAGAAUAUA | 1092 | (AGAC) | 20 | VQR- |
| | | | | | | (C12) | SpBE3 |
| W1332X | TGG | TAR | CUGAAUAUCAGCCAGAAUAU | 1093 | (AAG) | 20 | SpBE3 |
| | | | | | | (C13) | |
| W1332X | TGG | TAR | UCAGCCAGAAUAUAAGACAC | 1094 | (ACAAGT) | 20 (C6) | KKH- |
| | | | | | | | SaBE3 |
| Q1363X | CAA | TAA | AGUCAAGUUCCAAAUCGUUC | 1095 | (CGAA) | 20 (C4) | VQR- |
| | | | | | | | SpBE3 |
| Q1363X | CAA | TAA | AAGUCAAGUUCCAAAUCGUU | 1096 | (CCGAAT) | 20 (C5) | SaBE3 |
| Q1378X | CAA | TAA | UGAAUGUUAGUCAAAAUGUG | 1097 | (CGAT) | 20 | VQR- |
| | | | | | | (C12) | SpBE3 |
| Q1378X | CAA | TAA | AUGUUAGUCAAAAUGUGCGA | 1098 | (TGG) | 20(C9) | SpBE3 |
| Q1378X | CAA | TAA | UGUUAGUCAAAAUGUGCGAU | 1099 | (GGAA) | 20 (C8) | VQR- |
| | | | | | | | SpBE3 |
| Q1378X | CAA | TAA | UAUGAAUGUUAGUCAAAAUG | 1100 | (TGCGAT) | 20 | KKH- |
| | | | | | | (C14) | SaBE3 |
| R1381X | CGA | TGA | AAAUGUGCGAUGGAAAAACC | 1101 | (TGAA) | 20(C8) | VQR- |
| | | | | | | | SpBE3 |
| R1381X | CGA | TGA | UGUGCGAUGGAAAAACCUGA | 1102 | (AAG) | 20(C5) | SpBE3 |
| R1381X | CGA | TGA | GUGCGAUGGAAAAACCUGAA | 1103 | (AGTG) | 20(C4) | VQR- |
| | | | | | | | SpBE3 |
| R1381X | CGA | TGA | GCGAUGGAAAAACCUGAAAG | 1104 | (TGAA) | 20(C2) | VQR- |
| | | | | | | | SpBE3 |
| R1381X | CGA | TGA | AAUGUGCGAUGGAAAAACCU | 1105 | (GAAAGT) | 20(C7) | KKH- |
| | | | | | | | SaBE3 |
| W1382X | TGG | TAR | GGUUUUUCCAUCGCACAUUU | 1106 | (TGAC) | 20(C9) | VQR- |
| | | | | | | | SpBE3 |
| Q1401X | CAA | TAA | UAUUCUUAAAGGCAACUUUU | 1107 | (AAG) | 20 | SpBE3 |
| | | | | | | (C13) | |
| Q1401X | CAA | TAA | AUUCUUAAAGGCAACUUUUA | 1108 | (AGG) | 20 | SpBE3 |
| | | | | | | (C12) | |
| Q1401X | CAA | TAA | UUCUUAAAGGCAACUUUUAA | 1109 | (GGG) | 20 | SpBE3 |
| | | | | | | (C11) | |
| Q1401X | CAA | TAA | UCUUAAAGGCAACUUUUAAG | 1110 | (GGAT) | 20 | VQR- |
| | | | | | | (C10) | SpBE3 |
| Q1401X | CAA | TAA | UAAAGGCAACUUUUAAGGGA | 1111 | (TGG) | 20 (C7) | SpBE3 |
| Q1401X | CAA | TAA | AAAGGCAACUUUUAAGGGAU | 1112 | (GGAC) | 20 (C6) | VQR- |
| | | | | | | | SpBE3 |
| Q1401X | CAA | TAA | GGCAACUUUUAAGGGAUGGA | 1113 | (CGAT) | 20 (C3) | VQR- |
| | | | | | | | SpBE3 |
| Q1401X | CAA | TAA | AUUCUUAAAGGCAACUUUUA | 1114 | (AGGGAT) | 20 | SaBE3 |
| | | | | | | (C12) | |
| Q1401X | CAA | TAA | AAGGCAACUUUUAAGGGAUG | 1115 | (GACGAT) | 20 (C5) | KKH- |
| | | | | | | | SaBE3 |
| W1408X | TGG | TAR | AUCCCUUAAAAGUUGCCUUU | 1116 | (AAG) | 20 (C-1) | SpBE3 |
| W1408X | TGG | TAR | AAUAAUCGUCCAUCCCUUAA | 1117 | (AAG) | 20 | SpBE3 |
| | | | | | | (C11) | |
| W1408X | TGG | TAR | AUCCCUUAAAAGUUGCCUUU | 1118 | (AAGAAT) | 20 (C-1) | SaBE3 |
| W1408X | TGG | TAR | AUAAUAAUCGUCCAUCCCUU | 1119 | (AAAAGT) | 20 | KKH- |
| | | | | | | (C13) | SaBE3 |
| W1408X | TGG | TAR | CAUCCCUUAAAAGUUGCCUU | 1120 | (TAAGAAT) | 20 (C1) | St1BE3 |
| Q1424X | CAG | TAG | GUAGACAAGCAGCCCAAAUA | 1121 | (TGAA) | 20 | VQR- |
| | | | | | | (C10) | SpBE3 |
| Q1424X | CAG | TAG | AAGCAGCCCAAAUAUGAAUA | 1122 | (TAG) | 20 (C4) | SpBE3 |
| Q1462X | CAA | TAA | CAUAGAUAAUUUCAACCAAC | 1123 | (AGAA) | 20 | VQR- |
| | | | | | | (C13) | SpBE3 |
| Q1462X | CAA | TAA | AUAAUUUCAACCAACAGAAA | 1124 | (AAG) | 20 (C8) | SpBE3 |
| Q1462X | CAA | TAA | UAAUUUCAACCAACAGAAAA | 1125 | (AGAA) | 20 (C7) | VQR- |
| | | | | | | | SpBE3 |
| Q1462X | CAA | TAA | AUUUCAACCAACAGAAAAAG | 1126 | (AAG) | 20 (C5) | SpBE3 |
| Q1462X | CAA | TAA | UUUCAACCAACAGAAAAAGA | 1127 | (AGAT) | 20 (C4) | VQR- |
| | | | | | | | SpBE3 |
| Q1462X | CAA | TAA | AACCAACAGAAAAAGAAGAU | 1128 | (AAG) | 20 (C-1) | SpBE3 |
| Q1462X | CAA | TAA | AAUUUCAACCAACAGAAAAA | 1129 | (GAAGAT) | 20 (C6) | KKH- |
| | | | | | | | SaBE3 |
| Q1462X | CAA | TAA | UCAACCAACAGAAAAAGAAG | 1130 | (ATAAGT) | 20 (C2) | KKH- |
| | | | | | | | SaBE3 |
| Q1462X | CAA | TAA | GAUAAUUUCAACCAACAGAA | 1131 | (AAAGAAG) | 20 (C9) | St1BE3 |
| Q1463X | CAG | TAG | AUUUCAACCAACAGAAAAAG | 1132 | (AAG) | 20 | SpBE3 |
| | | | | | | (C12) | |
| Q1463X | CAG | TAG | UUUCAACCAACAGAAAAAGA | 1133 | (AGAT) | 20 | VQR- |
| | | | | | | (C11) | SpBE3 |
| Q1463X | CAG | TAG | AACCAACAGAAAAAGAAGAU | 1134 | (AAG) | 20 (C7) | SpBE3 |
| Q1463X | CAG | TAG | AAUUUCAACCAACAGAAAAA | 1135 | (GAAGAT) | 20 | KKH- |
| | | | | | | (C13) | SaBE3 |
| Q1463X | CAG | TAG | UCAACCAACAGAAAAAGAAG | 1136 | (ATAAGT) | 20(C9) | KKH- |
| | | | | | | | SaBE3 |
| Q1463X | CAG | TAG | AGAAAAAGAAGAUAAGUAUU | 1137 | (TCAAAT) | 20 (C-1) | KKH- |
| | | | | | | | SaBE3 |
| Q1470X | CAA | TAA | UGGAGGUCAAGACAUCUUUA | 1138 | (TGAC) | 20 (C8) | VQR- |
| | | | | | | | SpBE3 |
| Q1470X | CAA | TAA | AGGUCAAGACAUCUUUAUGA | 1139 | (CAG) | 20 (C5) | SpBE3 |
| Q1470X | CAA | TAA | GGUCAAGACAUCUUUAUGAC | 1140 | (AGAA) | 20 (C4) | VQR- |
| | | | | | | | SpBE3 |
| Q1470X | CAA | TAA | UCAAGACAUCUUUAUGACAG | 1141 | (AAG) | 20 (C2) | SpBE3 |
| Q1470X | CAA | TAA | CAAGACAUCUUUAUGACAGA | 1142 | (AGAA) | 20 (C1) | VQR- |
| | | | | | | | SpBE3 |
| Q1470X | CAA | TAA | GAGGUCAAGACAUCUUUAUG | 1143 | (ACAGAAG) | 20 (C6) | St1BE3 |
| Q1470X | CAA | TAA | GUCAAGACAUCUUUAUGACA | 1144 | (GAAGAAC) | 20 (C3) | St1BE3 |
| Q1478X | CAG | TAG | CAGAAGAACAGAAGAAAUAC | 1145 | (TATAAT) | 20 (C9) | KKH- |
| | | | | | | | SaBE3 |
| Q1478X | CAG | TAG | GAACAGAAGAAAUACUAUAA | 1146 | (TGCAAT) | 20 (C4) | KKH- |
| | | | | | | | SaBE3 |
| Q1494X | CAA | TAA | AGCCACAAAAGCCAAUUCCU | 1147 | (CGAC) | 20 (C6) | VQR- |
| | | | | | | | SpBE3 |
| Q1494X | CAA | TAA | ACAAAAGCCAAUUCCUCGAC | 1148 | (CAG) | 20 (C2) | SpBE3 |
| Q1494X | CAA | TAA | CAAAAGCCAAUUCCUCGACC | 1149 | (AGG) | 20 (C1) | SpBE3 |
| Q1494X | CAA | TAA | AAAAGCCAAUUCCUCGACCA | 1150 | (GGG) | 20 (C-1) | SpBE3 |
| Q1494X | CAA | TAA | AAAGCCAAUUCCUCGACCAG | 1151 | (GGG) | 20 (C-2) | SpBE3 |
| R1499X | CGA | TGA | AAUUCCUCGACCAGGGGUAA | 1152 | (AAAAAT) | 20 (C8) | KKH- |
| | | | | | | | SaBE3 |
| Q1505X | CAA | TAA | AAAAUCCAAGGAUGUAUAUU | 1153 | (TGAC) | 20 (C7) | VQR- |
| | | | | | | | SpBE3 |
| 01505X | CAA | TAA | CCAAGGAUGUAUAUUUGACC | 1154 | (TAG) | 20(C2) | SpBE3 |
| Q1505X | CAA | TAA | CAAGGAUGUAUAUUUGACCU | 1155 | (AGTG) | 20 (C1) | VQR- |
| | | | | | | | SpBE3 |
| Q1505X | CAA | TAA | AUCCAAGGAUGUAUAUUUGA | 1156 | (CCTAGT) | 20(C4) | KKH- |
| | | | | | | | SaBE3 |
| Q1515X | CAA | TAA | CUAGUGACAAAUCAAGCCUU | 1157 | (TGAT) | 20 (C8) | VQR- |
| | | | | | | | SpBE3 |
| Q1515X | CAA | TAA | ACAAAUCAAGCCUUUGAUAU | 1158 | (TAG) | 20 (C2) | SpBE3 |
| Q1515X | CAA | TAA | ACCUAGUGACAAAUCAAGCC | 1159 | (TTTGAT) | 20 | KKH- |
| | | | | | | (C10) | SaBE3 |
| Q1515X | CAA | TAA | UGACAAAUCAAGCCUUUGAU | 1160 | (ATTAGT) | 20 (C4) | KKH- |
| | | | | | | | SaBE3 |
| Q1539/41 | CAA | TAA | GGAGGGUCAAAGUCAACAUA | 1161 | (TGAC) | 20 (C8) | VQR- |
| X | | | | | | | SpBE3 |
| Q1539/41 | CAA | TAA | GGUCAAAGUCAACAUAUGAC | 1162 | (TGAA) | 20 | VQR- |
| X | | | | | | (C4/10) | SpBE3 |
| Q1539/41 | CAA | TAA | UCAAAGUCAACAUAUGACUG | 1163 | (AAG) | 20 | SpBE3 |
| X | | | | | | (C2/8) | |
| Q1539/41 | CAA | TAA | GGUCAAAGUCAACAUAUGAC | 1164 | (TGAAGT) | 20 | KKH- |
| X | | | | | | (C4/10) | SaBE3 |
| Q1541X | CAA | TAA | GGUCAAAGUCAACAUAUGAC | 1165 | (TGAA) | 20 | VQR- |
| | | | | | | (C10) | SpBE3 |
| Q1541X | CAA | TAA | UCAAAGUCAACAUAUGACUG | 1166 | (AAG) | 20 (C8) | SpBE3 |
| Q1541X | CAA | TAA | GGUCAAAGUCAACAUAUGAC | 1167 | (TGAAGT) | 20 | KKH- |
| | | | | | | (C4/10) | SaBE3 |
| W1549X | TGG | TAR | AUUUAUCCAAUAUAAAACUU | 1168 | (CAG) | 20 (C8) | SpBE3 |
| W1549X | TGG | TAR | ACAUUUAUCCAAUAUAAAAC | 1169 | (TTCAGT) | 20 | KKH- |
| | | | | | | (C10) | SaBE3 |
| W1578X | TGG | TAR | CCAUCCUACAGUGAAGUAGU | 1170 | (AGTG) | 20(C2) | VQR- |
| | | | | | | | SpBE3 |
| W1578X | TGG | TAR | UCCAUCCUACAGUGAAGUAG | 1171 | (TAG) | 20(C3) | SpBE3 |
| W1578X | TGG | TAR | UAUUCCAUCCUACAGUGAAG | 1172 | (TAG) | 20(C6) | SpBE3 |
| W1578X | TGG | TAR | AAAUAUUCCAUCCUACAGUG | 1173 | (AAG) | 20(C9) | SpBE3 |
| W1578X | TGG | TAR | AAAAAUAUUCCAUCCUACAG | 1174 | (TGAA) | 20 | VQR- |
| | | | | | | (C11) | SpBE3 |
| W1578X | TGG | TAR | UCAAAAAUAUUCCAUCCUAC | 1175 | (AGTG) | 20 | VQR- |
| | | | | | | (C13) | SpBE3 |
| W1578X | TGG | TAR | AUUCCAUCCUACAGUGAAGU | 1176 | (AGTAGT) | 20(C5) | KKH- |
| | | | | | | | SaBE3 |
| W1578X | TGG | TAR | AAUAUUCCAUCCUACAGUGA | 1177 | (AGTAGT) | 20(C8) | KKH- |
| | | | | | | | SaBE3 |
| W1578X | TGG | TAR | AAAAAUAUUCCAUCCUACAG | 1178 | (TGAAGT) | 20 | KKH- |
| | | | | | | (C11) | SaBE3 |
| W1578X | TGG | TAR | AAAUCAAAAAUAUUCCAUCC | 1179 | (TACAGT) | 20 | KKH- |
| | | | | | | (C16) | SaBE3 |
| R1610X | CGA | TGA | UUCCGAGUGAUCCGUCUUGC | 1180 | (CAG) | 20(C4) | SpBE3 |
| R1610X | CGA | TGA | UCCGAGUGAUCCGUCUUGCC | 1181 | (AGG) | 20(C3) | SpBE3 |
| R1610X | CGA | TGA | CCGAGUGAUCCGUCUUGCCA | 1182 | (GGAT) | 20(C2) | VQR- |
| | | | | | | | SpBE3 |
| R1610X | CGA | TGA | UUCCGAGUGAUCCGUCUUGC | 1183 | (CAGGAT) | 20(C4) | SaBE3 |
| R1619X | CGA | TGA | GAUUGGCCGAAUCCUACGUC | 1184 | (TAG) | 20(C8) | SpBE3 |
| R1619X | CGA | TGA | CCGAAUCCUACGUCUAGUCA | 1185 | (AAG) | 20(C2) | SpBE3 |
| R1619X | CGA | TGA | CGAAUCCUACGUCUAGUCAA | 1186 | (AGG) | 20(C1) | SpBE3 |
| R1619X | CGA | TGA | GAAUCCUACGUCUAGUCAAA | 1187 | (GGAG) | 20(C-1) | EQR- |
| | | | | | | | SpBE3 |
| R1619X | CGA | TGA | AGGAUUGGCCGAAUCCUACG | 1188 | (TCTAGT) | 20 | KKH- |
| | | | | | | (C10) | SaBE3 |
| R1619X | CGA | TGA | CGAAUCCUACGUCUAGUCAA | 1189 | (AGGAG) | 20 (C1) | St3BE3 |
| Q1693X | CAA | TAA | UUCCAAAUUACAACCUCUGC | 1190 | (TGG) | 20 (C4) | SpBE3 |
| Q1693X | CAA | TAA | AAAUUACAACCUCUGCUGGC | 1191 | (TGG) | 20 (C-1) | SpBE3 |
| W1700X | TGG | TAR | AGCCAGCAGAGGUUGUAAUU | 1192 | (TGG) | 20 (C1) | SpBE3 |
| W1700X | TGG | TAR | CAAUCCAUCCCAGCCAGCAG | 1193 | (AGG) | 20 | SpBE3 |
| | | | | | | (C11) | |
| W1700X | TGG | TAR | GCAAUCCAUCCCAGCCAGCA | 1194 | (GAG) | 20 | SpBE3 |
| | | | | | | (C12) | |
| W1700X | TGG | TAR | AGCAAUCCAUCCCAGCCAGC | 1195 | (AGAG) | 20 | EQR- |
| | | | | | | (C13) | SpBE3 |
| W1700X | TGG | TAR | CCAUCCCAGCCAGCAGAGGU | 1196 | (TGTAAT) | 20(C7) | KKH- |
| | | | | | | | SaBE3 |
| W1700X | TGG | TAR | AGCAAUCCAUCCCAGCCAGC | 1197 | (AGAGGT) | 20 | KKH- |
| | | | | | | (C13) | SaBE3 |
| W1786X | TGG | TAR | AAACCUCAUAGAACAUCUCA | 1198 | (AAG) | 20 (C-1) | SpBE3 |
| W1786X | TGG | TAR | AAACUUCUCCCAAACCUCAU | 1199 | (AGAA) | 20 | VQR- |
| | | | | | | (C11) | SpBE3 |
| W1786X | TGG | TAR | CAAACUUCUCCCAAACCUCA | 1200 | (TAG) | 20 | SpBE3 |
| | | | | | | (C12) | |
| W1786X | TGG | TAR | CCAAACCUCAUAGAACAUCU | 1201 | (CAAAGT) | 20 (C2) | KKH- |
| | | | | | | | SaBE3 |
| W1786X | TGG | TAR | UCAAACUUCUCCCAAACCUC | 1202 | (ATAGAAC) | 20 | St1BE3 |
| | | | | | | (C13) | |
| Q1795X | CAG | TAG | CCCGAUGCGACCCAGUUUAU | 1203 | (AGAG) | 20 | EQR- |
| | | | | | | (C13) | SpBE3 |
| Q1795X | CAG | TAG | CCGAUGCGACCCAGUUUAUA | 1204 | (GAG) | 20 | SpBE3 |
| | | | | | | (C12) | |
| Q1822X | CAG | TAG | CAAAGUCCAGCUCAUUGCCA | 1205 | (TGG) | 20(C8) | SpBE3 |
| Q1822X | CAG | TAG | AAAGUCCAGCUCAUUGCCAU | 1206 | (GGAT) | 20 (C7) | VQR- |
| | | | | | | | SpBE3 |
| Q1822X | CAG | TAG | ACAAAGUCCAGCUCAUUGCC | 1207 | (ATGGAT) | 20(C9) | SaBE3 |
| Q1862X | CAG | TAG | UCUCUUCGUUCACAGAUGGA | 1208 | (AGAA) | 20 | VQR- |
| | | | | | | (C13) | SpBE3 |
| Q1862X | CAG | TAG | CUUCGUUCACAGAUGGAAGA | 1209 | (AAG) | 20 | SpBE3 |
| | | | | | | (C10) | |
| Q1862X | CAG | TAG | UUCGUUCACAGAUGGAAGAA | 1210 | (AGG) | 20 (C9) | SpBE3 |
| Q1862X | CAG | TAG | UCUUCGUUCACAGAUGGAAG | 1211 | (AAAGGT) | 20 | KKH- |
| | | | | | | (C11) | SaBE3 |
| Q1888X | CAA | TAA | CUAAAACGGAAACAAGAGGA | 1212 | (TGTG) | 20 | VQR- |
| | | | | | | (C13) | SpBE3 |
| Q1897X | CAG | TAG | ACUGUCAUUCAGCGUGCUUA | 1213 | (TAG) | 20 | SpBE3 |
| | | | | | | (C10) | |
| Q1897X | CAG | TAG | CUGUCAUUCAGCGUGCUUAU | 1214 | (AGAC) | 20(C9) | VQR- |
| | | | | | | | SpBE3 |
| Q1907X | CAA | TAA | CAAAAUGUCAAAAAUAUAUC | 1215 | (AAG) | 20 (C1) | SpBE3 |
| Q1907X | CAA | TAA | ACCGCUUAAGGCAAAAUGUC | 1216 | (AAT) | 20 | KKH- |
| | | | | | | (C12) | SaBE3 |
| Q1907X | CAA | TAA | GGCAAAAUGUCAAAAAUAUA | 1217 | (TCAAGT) | 20 (C3) | KKH- |
| | | | | | | | SaBE3 |
| Q1971X | CAA | TAA | GAAAUAUGAACAAGACAGAA | 1218 | (CAG) | 20 | SpBE3 |
| | | | | | | (C11) | |
| Q1971X | CAA | TAA | AAAUAUGAACAAGACAGAAC | 1219 | (AGAA) | 20 | VQR- |
| | | | | | | (C10) | SpBE3 |
| Q1971X | CAA | TAA | AUGAACAAGACAGAACAGAA | 1220 | (AAG) | 20 (C6) | SpBE3 |
| Q1971X | CAA | TAA | UGAACAAGACAGAACAGAAA | 1221 | (AGG) | 20 (C5) | SpBE3 |
| Q1971X | CAA | TAA | GAACAAGACAGAACAGAAAA | 1222 | (GGAA) | 20 (C4) | VQR- |
| | | | | | | | SpBE3 |
| Q1971X | CAA | TAA | ACAAGACAGAACAGAAAAGG | 1223 | (AAG) | 20 (C2) | SpBE3 |
| Q1971X | CAA | TAA | CAAGACAGAACAGAAAAGGA | 1224 | (AGAC) | 20 (C1) | VQR- |
| | | | | | | | SpBE3 |
| Q1971X | CAA | TAA | AGAAAUAUGAACAAGACAGA | 1225 | (ACAGAAA) | 20 | St1BE3 |
| | | | | | | (C12) | |

^{a}BE types: SpBE3 = APOBEC1-SpCas9n-UGI; VQR-SpBE3 = APOBEC1-VQR-SpCas9n-UGI; EQR-SpBE3 = APOBEC1-EQR-SpCas9n-UGI; VRER-SpBE3 = APOBEC1-VRER-SpCas9n-UGI; SaBE3 = APOBEC1-SaCas9n-UGI; KKH-SaBE3 = APOBEC1-KKH-SaCas9n-UGI; St3BE3 = APOBEC1-St3Cas9n-UGI; St1BE3 = APOBEC1-St1Cas9n-UGI.

### Target Base in Non-Coding Region—Splicing Variants

Some aspects of the present disclosure provide strategies of reducing the activity of ion channels (e.g., ion channels in in DRG neurons) via preventing the ion channel mRNA maturation and production. In some embodiments, such strategies involve alterations of splicing sites in the ion channel gene. Altered splicing site may lead to altered splicing and maturation of the ion channel mRNA. For example, in some embodiments, an altered splicing site may lead to the skipping of an exon, in turn leading to a truncated protein product or an altered reading frame. In some embodiments, an altered splicing site may lead to translation of an intron sequence and premature translation termination when an in frame stop codon is encountered by the translating ribosome in the intron. In some embodiments, a start codon is edited and protein translation initiates at the next ATG codon, which may not be in the correct coding frame.

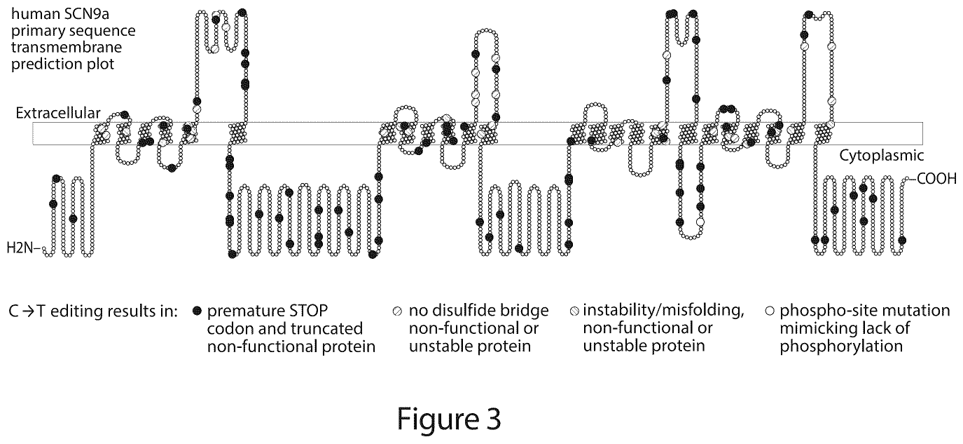

The splicing sites typically comprises an intron donor site, a Lariat branch point, and an intron acceptor site. The mechanism of splicing are familiar to those skilled in the art. As illustrated in FIG. 3, the intron donor site has a consensus sequence of GGGTRAGT, and the C bases paired with the G bases in the intron donor site consensus sequence may be targeted by a nucleobase editors described herein, thereby altering the intron donor site. The Lariat branch point also has consensus sequences, e.g., YTRAC, wherein Y is a pyrimidine, and R is a purine. The C base in the Lariat branch point consensus sequence may be targeted by the nucleobase editors, leading to the skipping of the following exon. The intron acceptor site has a consensus sequence of YNCAGG, wherein Y is a pyrimidine, and N is any nucleotide. The C base of the consensus sequence of the intron acceptor site, and the C base paired with the G bases in the consensus sequence of the intron acceptor site may be targeted by the nucleobase editors described herein, thereby altering the intron acceptor site, in turn leading the skipping of an exon. General strategies of altering the splicing sites of the ion channel gene are described in Table 5.

TABLE 5
Exemplary Alteration of Intron-Exon Junction via Base Editing

| | Consensus | Base-editing | Edited | |
|---|---|---|---|---|
| Target site | Sequence | reaction (s) | sequence | Outcome |
| Intron | GGGTRAGT | 2^{nd} or 3^{rd} base | GAGTRAGT | Intron sequence is |
| donor | (example) | C to T on | (example) | translated as exon, in frame |
| | | complementary | | premature STOP codon |
| | | strand | | |
| Lariat | YTRAC | 5^{th} base | YTRAT | The following exon is |
| branch | (example) | C to T on | (example) | skipped from the mature |
| point | | coding | | mRNA, which may affect |
| | | strand | | the coding frame |
| Intron | Y(rich)NCAGG | 2^{nd} to last base | Y(rich)NCAAG | The exon is skipped from |
| acceptor | (example) | C to T on | (example) | the mature mRNA, which |
| | | complementary | | may affect the coding frame |
| | | strand | | |
| Start | ATG (Met/M) | 3^{rd} base | ATA (Ile/I) | The next ATG is used as |
| codon | | C to T on | | start codon, which may |
| | | complementary | | affect the coding frame |
| | | strand | | |

Provided in Table 6 are non-limiting examples of alterations that may be made to non-coding regions (e.g., splicing sites) in the SCN9A gene using nucleobase editors and the guide sequences that may be used for each alteration.

TABLE 6
Alteration of Intron/Exon Junctions in NaV1.7 (SCN9A) Gene via Base Editing

| Target | Genome target seq./ | Programmable guide- | SEQ ID | | gRNA size | |
|---|---|---|---|---|---|---|
| site* | junction | RNA sequence | NOs | (PAM) | (C edited) | BE type^{a} |
| donor, | CTAGGTTGCAAgtaagtgccttt | UUACUUGCAACCUAGCCCGC | 1226 | (CGAT) | 20(C4) | VQR-SpBE3 |
| intron 1 | (SEQ ID NO: 1457) | | | | | |
| | | AAAGGCACUUACUUGCAACC | 1227 | (TAG) | 20(C12) | SpBE3 |
| | | ACUUACUUGCAACCUAGCCC | 1228 | (GCCGAT) | 20(C6) | KKH-SaBE3 |
| acceptor, | ctttgtttccatccagGCCTCTT | AGAGGCCUGGAUGGAAACAA | 1229 | (AGAA) | 20(C6/7) | VQR-SpBE3 |
| intron 1 | (SEQ ID NO: 1458) | | | | | |
| | | AAGAGGCCUGGAUGGAAACA | 1230 | (AAG) | 20(C7/8) | SpBE3 |
| | | AGAGGCCUGGAUGGAAACAA | 1231 | (AGAAAT) | 20(C6/7) | KKH-SaBE3 |
| | | UAAGAGGCCUGGAUGGAAAC | 1232 | (AAAGAAA) | 20(C8/9) | St1BE3 |
| donor, | CTATGCAGACAAAAAGgtgagtt | CCUUUUUGUCUGCAUAGUAG | 1233 | (GGG) | 20(C1/2) | SpBE3 |
| intron 2 | (SEQ ID NO: 1459) | | | | | |
| | | ACCUUUUUGUCUGCAUAGUA | 1234 | (GGG) | 20(C2/3) | SpBE3 |
| | | CACCUUUUUGUCUGCAUAGU | 1235 | (AGG) | 20(C3/4) | SpBE3 |
| | | UCACCUUUUUGUCUGCAUAG | 1236 | (TAG) | 20(C4/5) | SpBE3 |
| | | AACUCACCUUUUUGUCUGCA | 1237 | (TAG) | 20(C7/8) | SpBE3 |
| | | CACCUUUUUGUCUGCAUAGU | 1238 | (AGGGGT) | 20(C3/4) | SaBE3 |
| | | UAAACUCACCUUUUUGUCUG | 1239 | (CATAGT) | 20(C9/10) | KKH-SaBE3 |
| | | CACCUUUUUGUCUGCAUAGU | 1240 | (AGGGG) | 20(C3/4) | St3BE3 |
| acceptor, | ctttttcctcctgcagACTTTCA | UCUGCAGGAGGAAAAAGAAA | 1241 | (GGAT) | 20(C2) | VQR-SpBE3 |
| intron 2 | (SEQ ID NO: 1460) | | | | | |
| | | GUCUGCAGGAGGAAAAAGAA | 1242 | (AGG) | 20(C3) | SpBE3 |
| | | AGUCUGCAGGAGGAAAAAGA | 1243 | (AAG) | 20(C4) | SpBE3 |
| | | GAAAGUCUGCAGGAGGAAAA | 1244 | (AGAA) | 20(C7) | VQR-SpBE3 |
| | | UGAAAGUCUGCAGGAGGAAA | 1245 | (AAG) | 20(C8) | SpBE3 |
| | | UACUAUGAAAGUCUGCAGGA | 1246 | (GGAA) | 20(C13) | VQR-SpBE3 |
| | | AGUCUGCAGGAGGAAAAAGA | 1247 | (AAGGAT) | 20(C4) | SaBE3 |
| | | AUGAAAGUCUGCAGGAGGAA | 1248 | (AAAGAAA) | 20(C9) | St1BE3 |
| donor, | TAGTACACTCatatccttttaaaaat | CACUCAUAUCCUUUUAAAAA | 1249 | (TGAT) | 20(C3/5) | VQR-SpBE3 |
| intron 3 | (SEQ ID NO: 1461) | | | | | |
| | | UAGUACACUCAUAUCCUUUU | 1250 | (AAAAAT) | 20(CE00) | KKH-SaBE3 |
| | | UACACUCAUAUCCUUUUAAA | 1251 | (AATGAT) | 20(C5/7) | KKH-SaBE3 |
| acceptor, | tgattctaagctacCTTATTCAG | UGAUUCUAAGCUACCUUAUU | 1252 | (CAG) | 20(C11/14) | SpBE3 |
| intron 3 | (SEQ ID NO: 1462) | | | | | |
| donor, | CCAAAAATGTCGAgtaagtgggt | CUUACUCGACAUUUUUGGUC | 1253 | (CAG) | 20(C5) | SpBE3 |
| intron 4 | (SEQ ID NO: 1463) | | | | | |
| | | ACCCACUUACUCGACAUUUU | 1254 | (TGG) | 20(C10) | SpBE3 |
| | | ACUCGACAUUUUUGGUCCAG | 1255 | (TCCGGT) | 20(C2) | KKH-SaBE3 |
| | | CACUUACUCGACAUUUUUGG | 1256 | (TCCAGT) | 20(C7) | KKH-SaBE3 |
| | | AUACCCACUUACUCGACAUU | 1257 | (TTTGGT) | 20(C12) | KKH-SaBE3 |
| acceptor, | atcttgtgtttagGTACACTTTTA | CCUAAACACAAGAUUCCAUU | 1258 | (GGG) | 20(C1/2) | SpBE3 |
| intron 4 | (SEQ ID NO: 1464) | | | | | |
| | | ACCUAAACACAAGAUUCCAU | 1259 | (TGG) | 20(C2/3) | SpBE3 |
| | | UAAAAGUGUACCUAAACACA | 1260 | (AGAT) | 20(C11/12) | VQR-SpBE3 |
| | | GUAAAAGUGUACCUAAACAC | 1261 | (AAG) | 20(C12/13) | SpBE3 |
| | | ACCUAAACACAAGAUUCCAU | 1262 | (TGGGAT) | 20(C2/3) | SaBE3 |
| | | AGUAAAAGUGUACCUAAACA | 1263 | (CAAGAT) | 20(C13/14) | KKH-SaBE3 |
| donor, | ATTGTTTTTGCgtaagtactttcagc | UACUUACGCAAAAACAAUGA | 1264 | (CGAC) | 20(C7) | VQR-SpBE3 |
| intron 5 | (SEQ ID NO: 1465) | | | | | |
| | | AAGUACUUACGCAAAAACAA | 1265 | (TGAC) | 20(C10) | VQR-SpBE3 |
| | | UUACGCAAAAACAAUGACGA | 1266 | (CAAAAT) | 20(C4) | KKH-SaBE3 |
| | | GCUGAAAGUACUUACGCAAA | 1267 | (AACAAT) | 20(C15) | KKH-SaBE3 |
| acceptor, | atttaattctacagGTATTTAACAGA | AAUACCUGUAGAAUUAAAUC | 1268 | (AGAA) | 20(C5/6) | VQR-SpBE3 |
| intron 5 | (SEQ ID NO: 1466) | | | | | |
| | | AAAUACCUGUAGAAUUAAAU | 1269 | (CAG) | 20(C6/7) | SpBE3 |
| | | AAAUACCUGUAGAAUUAAAU | 1270 | (CAGAAT) | 20(C6/7) | SaBE3 |
| | | UCUGUUAAAUACCUGUAGAA | 1271 | (TTAAAT) | 20(C12/13) | KKH-SaBE3 |
| | | UAAAUACCUGUAGAAUUAAA | 1272 | (TCAGAAT) | 20(C7/8) | St1BE3 |
| donor, | CTGTAATCCCAGgtaagaagtaa | CUUACCUGGGAUUACAGAAA | 1273 | (TAG) | 20(C5/6) | SpBE3 |
| intron 6 | (SEQ ID NO: 1467) | | | | | |
| | | UACUUCUUACCUGGGAUUAC | 1274 | (AGAA) | 20(C10/11) | VQR-SpBE3 |
| | | UUACUUCUUACCUGGGAUUA | 1275 | (CAG) | 20(C11/12) | SpBE3 |
| | | UUCUUACCUGGGAUUACAGA | 1276 | (AATAGT) | 20(C7/8) | KKH-SaBE3 |
| | | UACUUCUUACCUGGGAUUAC | 1277 | (AGAAAT) | 20(C10/11) | KKH-SaBE3 |
| | | AUUACUUCUUACCUGGGAUU | 1278 | (ACAGAAA) | 20(C12/13) | St1BE3 |
| acceptor, | ctcccattttcagGCCTGAAGAC | GCCUGAAAAUGGGAGAAAAA | 1279 | (AGTG) | 20(C2/3) | VQR-SpBE3 |
| intron 6 | (SEQ ID NO: 1468) | | | | | |
| | | GGCCUGAAAAUGGGAGAAAA | 1280 | (AAG) | 20(C3/4) | SpBE3 |
| | | UCUUCAGGCCUGAAAAUGGG | 1281 | (AGAA) | 20(C9/10) | VQR-SpBE3 |
| | | GUCUUCAGGCCUGAAAAUGG | 1282 | (GAG) | 20(C10/11) | SpBE3 |
| | | UGUCUUCAGGCCUGAAAAUG | 1283 | (GGAG) | 20(C11/12) | EQR-SpBE3 |
| | | UUGUCUUCAGGCCUGAAAAU | 1284 | (GGG) | 20(C12/13) | SpBE3 |
| | | CAGGCCUGAAAAUGGGAGAA | 1285 | (AAAAGT) | 20(C5/6) | KKH-SaBE3 |
| | | UGUCUUCAGGCCUGAAAAUG | 1286 | (GGAGAAA) | 20(C11/12) | St1BE3 |
| | | UUGUCUUCAGGCCUGAAAAU | 1287 | (GGGAG) | 20(C12/13) | St3BE3 |
| acceptor, | ttcttcttcaacagAATATTTTTA | CUGUUGAAGAAGAAUUUGAA | 1288 | (CAG) | 20(C1) | SpBE3 |
| intron 7 | (SEQ ID NO: 1469) | | | | | |
| | | UAUUCUGUUGAAGAAGAAUU | 1289 | (TGAA) | 20(C5) | VQR-SpBE3 |
| | | UAAAAAUAUUCUGUUGAAGA | 1290 | (AGAA) | 20(C11) | VQR-SpBE3 |
| | | AUAAAAAUAUUCUGUUGAAG | 1291 | (AAG) | 20(C12) | SpBE3 |
| | | UUCUGUUGAAGAAGAAUUUG | 1292 | (AACAGT) | 20(C3) | KKH-SaBE3 |
| | | AUAAAAAUAUUCUGUUGAAG | 1293 | (AAGAAT) | 20(C12) | SaBE3 |
| | | AAUAAAAAUAUUCUGUUGAA | 1294 | (GAAGAAT) | 20(C13) | St1BE3 |
| | | AGUAAUAAAAAUAUUCUGUU | 1295 | (GAAGAAG) | 20(C16) | St1BE3 |
| donor, | CACAGATTCAGGgtatgtaatatt | UACAUACCCUGAAUCUGUGC | 1296 | (TGAA) | 20(C7/8) | VQR-SpBE3 |
| intron 8 | (SEQ ID NO: 1470) | | | | | |
| | | AAUAUUACAUACCCUGAAUC | 1297 | (TGTG) | 20(C12/13) | VQR-SpBE3 |
| acceptor, | ctttctcgtgtgtagTCAGTGTC | ACACUGACUACACACGAGAA | 1298 | (AGAA) | 20(C8) | VQR-SpBE3 |
| intron 8 | (SEQ ID NO: 1471) | | | | | |
| | | GACACUGACUACACACGAGA | 1299 | (AAG) | 20(C9) | SpBE3 |
| | | CUGGACACUGACUACACACG | 1300 | (AGAA) | 20(C12) | VQR-SpBE3 |
| donor, | CTTTACCAACAGgtgagtaccaa | CCUGUUGGUAAAGGUUUUCC | 1301 | (CAG) | 20(C1/2) | SpBE3 |
| intron 9 | (SEQ ID NO: 1472) | | | | | |
| | | UGGUACUCACCUGUUGGUAA | 1302 | (AGG) | 20(C10/11) | SpBE3 |
| | | UUGGUACUCACCUGUUGGUA | 1303 | (AAG) | 20(C11/12) | SpBE3 |
| | | CACCUGUUGGUAAAGGUUUU | 1304 | (CCCAGT) | 20(C3/4) | KKH-SaBE3 |
| | | CUUGGUACUCACCUGUUGGU | 1305 | (AAAGGT) | 20(C12/13) | KKH-SaBE3 |
| acceptor, | ccatttttccctagACGCTGCGT | CGCAGCGUCUAGGGAAAAAU | 1306 | (GGAA) | 20(C9) | VQR-SpBE3 |
| intron 9 | (SEQ ID NO: 1473) | | | | | |
| | | ACGCAGCGUCUAGGGAAAAA | 1307 | (TGG) | 20(C10) | SpBE3 |
| | | CGCAGCGUCUAGGGAAAAAU | 1308 | (GGAAAT) | 20(C9) | KKH-SaBE3 |
| | | GCAGCACGCAGCGUCUAGGG | 1309 | (AAAAAT) | 20(C15) | KKH-SaBE3 |
| acceptor, | cttggcccaaccagGCAATTGCA | GCAAUUGCCUGGUUGGGCCA | 1310 | (AGAC) | 20(C8/9) | VQR-SpBE3 |
| intron 10 | (SEQ ID NO: 1474) | | | | | |
| | | UGCAAUUGCCUGGUUGGGCC | 1311 | (AAG) | 20(C9/10) | SpBE3 |
| donor, | CCCCCAATCAGgtaccacccaaa | GGUGGUACCUGAUUGGGGGU | 1312 | (AGAC) | 20(C8/9) | VQR-SpBE3 |
| intron 11 | (SEQ ID NO: 1475) | | | | | |
| | | GGGUGGUACCUGAUUGGGGG | 1313 | (TAG) | 20(C9/10) | SpBE3 |
| | | UUUGGGUGGUACCUGAUUGG | 1314 | (GGG) | 20(C12/13) | SpBE3 |
| | | AAUUUGGGUGGUACCUGAUU | 1315 | (GGGGGT) | 20(C14/15) | SaBE3 |
| | | AAUUUGGGUGGUACCUGAUU | 1316 | (GGGGG) | 20(C14/15) | St3BE3 |
| acceptor, | atttttctgcagTCACCACTCAGCAT | AUGCUGAGUGGUGACUGCAG | 1317 | (AAAAAT) | 20(C15) | KKH-SaBE3 |
| intron 11 | (SEQ ID NO: 1476) | | | | | |
| donor, | TTCTGCCAGAGgtgataatagata | UCUAUUAUCACCUCUGGCAG | 1318 | (AAG) | 20(C11/12) | SpBE3 |
| intron 12a | (SEQ ID NO: 1477) | | | | | |
| | | UAUCUAUUAUCACCUCUGGC | 1319 | (AGAA) | 20(C13/14) | VQR-SpBE3 |
| | | CUUAUCUAUUAUCACCUCUG | 1320 | (GCAGAAG) | 20(C15/16) | St1BE3 |
| donor, | CTGATGACAGCgtaaggacg | CGUCCUUACGCUGUCAUCAG | 1321 | (AAG) | 20(C9) | SpBE3 |
| intron 12b | (SEQ ID NO: 1478) | | | | | |
| | | AACGUCCUUACGCUGUCAUC | 1322 | (AGAA) | 20(C11) | VQR-SpBE3 |
| | | AAACGUCCUUACGCUGUCAU | 1323 | (CAG) | 20(C12) | SpBE3 |
| | | AACGUCCUUACGCUGUCAUC | 1324 | (AGAAGT) | 20(C11) | KKH-SaBE3 |
| | | AAAACGUCCUUACGCUGUCA | 1325 | (TCAGAAG) | 20(C13) | St1BE3 |
| acceptor, | attgattttttttttagGGCACGACC | GUGCCCUAAAAAAAAAAUCA | 1326 | (ATTAAT) | 20(C5/6) | KKH-SaBE3 |
| intron 13 | (SEQ ID NO: 1479) | | | | | |
| | | GGUCGUGCCCUAAAAAAAAA | 1327 | (ATCAAT) | 20(C9/10) | KKH-SaBE3 |
| | | GAUUGGUCGUGCCCUAAAAA | 1328 | (AAAAAT) | 20(C13/14) | KKH-SaBE3 |
| donor, | CACTGTGGAAGgtatgtaataatc | GAUUAUUACAUACCUUCCAC | 1329 | (AGTG) | 20(C13/14) | VQR-SpBE3 |
| intron 13 | (SEQ ID NO: 1480) | | | | | |
| | | ACAUACCUUCCACAGUGUUU | 1330 | (GTTAAT) | 20(C6/7) | KKH-SaBE3 |
| acceptor, | cttttttctcccagAACTTGAAG | GUUCUGGGAGAAAAAAGCAG | 1331 | (AGAA) | 20(C4) | VQR-SpBE3 |
| intron 13 | (SEQ ID NO: 1481) | | | | | |
| | | AGUUCUGGGAGAAAAAAGCA | 1332 | (GAG) | 20(C5) | SpBE3 |
| | | AAGUUCUGGGAGAAAAAAGC | 1333 | (AGAG) | 20(C6) | EQR-SpBE3 |
| | | CAAGUUCUGGGAGAAAAAAG | 1334 | (CAG) | 20(C7) | SpBE3 |
| | | UCAAGUUCUGGGAGAAAAAA | 1335 | (GCAG) | 20(C8) | FALSE |
| | | CUUCAAGUUCUGGGAGAAAA | 1336 | (AAG) | 20(C10) | SpBE3 |
| | | AAGUUCUGGGAGAAAAAAGC | 1337 | (AGAGAAC) | 20(C6) | St1BE3 |
| donor, | CTATAGGAAATTTGgtaagtctc | CUUACCAAAUUUCCUAUAGC | 1338 | (AAG) | 20(C1/2) | SpBE3 |
| intron 14 | (SEQ ID NO: 1482) | | | | | |
| | | GAGACUUACCAAAUUUCCUA | 1339 | (TAG) | 20(C5/6) | SpBE3 |
| | | GACUUACCAAAUUUCCUAUA | 1340 | (GCAAGT) | 20(C7/8) | KKH-SaBE3 |
| acceptor, | atttttctcacttagGTCTTTACTGG | UUCCAGUAAAGACCUAAGUG | 1341 | (AGAA) | 20(C13/14) | VQR-SpBE3 |
| intron 14 | (SEQ ID NO: 1483) | | | | | |
| | | GAUUCCAGUAAAGACCUAAG | 1342 | (TGAG) | 20(C13/14) | EQR-SpBE3 |
| | | GUAAAGACCUAAGUGAGAAA | 1343 | (AATAAT) | 20(C8/9) | KKH-SaBE3 |
| | | CCAGUAAAGACCUAAGUGAG | 1344 | (AAAAAT) | 20(C11/12) | KKH-SaBE3 |
| | | GAUUCCAGUAAAGACCUAAG | 1345 | (TGAGAAA) | 20(C15/16) | St1BE3 |
| donor, | ATCATTCAGACTGgtaaacataaa | UUACCAGUCUGAAUGAUCGC | 1346 | (AGAA) | 20(C4/5) | VQR-SpBE3 |
| intron 15 | (SEQ ID NO: 1484) | | | | | |
| | | UUUACCAGUCUGAAUGAUCG | 1347 | (CAG) | 20(C5/6) | SpBE3 |
| | | UUUAUGUUUACCAGUCUGAA | 1348 | (TGAT) | 20(C11/12) | VQR-SpBE3 |
| | | AGUUUAUGUUUACCAGUCUG | 1349 | (AATGAT) | 20(C13/14) | KKH-SaBE3 |
| | | GUUUACCAGUCUGAAUGAUC | 1350 | (GCAGAAC) | 20(C6/7) | St1BE3 |
| acceptor, | actttatatttgcttttagCTCCGAG | CGGAGCUAAAAGCAAAUAUA | 1351 | (AAG) | 20(C6) | SpBE3 |
| intron 15 | (SEQ ID NO: 1485) | | | | | |
| | | AGCUAAAAGCAAAUAUAAAG | 1352 | (TTTAAT) | 20(C3) | KKH-SaBE3 |
| | | CUCGGAGCUAAAAGCAAAUA | 1353 | (TAAAGT) | 20(C8) | KKH-SaBE3 |
| | | UUGAAGACUCGGAGCUAAAA | 1354 | (GCAAAT) | 20(C15) | KKH-SaBE3 |
| donor, | ATTGGAAACCTGGTGgtatgtaacca | CACCAGGUUUCCAAUGACCA | 1355 | (TGAC) | 20(C1) | VQR-SpBE3 |
| intron 16 | (SEQ ID NO: 1486) | | | | | |
| | | ACAUACCACCAGGUUUCCAA | 1356 | (TGAC) | 20 (C7) | VQR-SpBE3 |
| | | UGGUUACAUACCACCAGGUU | 1357 | (TCCAAT) | 20(C12) | KKH-SaBE3 |
| acceptor, | ccaccctgatatagGTCCTAAAC | CUAUAUCAGGGUGGGGAGAG | 1358 | (GGG) | 20(C1/2) | SpBE3 |
| intron 16 | (SEQ ID NO: 1487) | | | | | |
| | | CCUAUAUCAGGGUGGGGAGA | 1359 | (GGG) | 20(C2/3) | SpBE3 |
| | | ACCUAUAUCAGGGUGGGGAG | 1360 | (AGG) | 20(C3/4) | SpBE3 |
| | | GACCUAUAUCAGGGUGGGGA | 1361 | (GAG) | 20(C4/5) | SpBE3 |
| | | GGACCUAUAUCAGGGUGGGG | 1362 | (AGAG) | 20(C5/6) | EQR-SpBE3 |
| | | AGGACCUAUAUCAGGGUGGG | 1363 | (GAG) | 20(C6/7) | SpBE3 |
| | | UAGGACCUAUAUCAGGGUGG | 1364 | (GGAG) | 20(C7/8) | EQR-SpBE3 |
| | | UUAGGACCUAUAUCAGGGUG | 1365 | (GGG) | 20(C8/9) | SpBE3 |
| | | UUUAGGACCUAUAUCAGGGU | 1366 | (GGG) | 20(C9/10) | SpBE3 |
| | | GUUUAGGACCUAUAUCAGGG | 1367 | (TGG) | 20(C10/11) | SpBE3 |
| | | AGGUUUAGGACCUAUAUCAG | 1368 | (GGTG) | 20(C12/13) | VQR-SpBE3 |
| | | CCUAUAUCAGGGUGGGGAGA | 1369 | (GGGGGT) | 20(C2/3) | SaBE3 |
| | | AAUAGGUUUAGGACCUAUAU | 1370 | (CAGGGT) | 20(C15/16) | SaBE3 |
| | | CCUAUAUCAGGGUGGGGAGA | 1371 | (GGGGG) | 20(C2/3) | St3BE3 |
| | | ACCUAUAUCAGGGUGGGGAG | 1372 | (AGGGG) | 20(C3/4) | St3BE3 |
| | | UUAGGACCUAUAUCAGGGUG | 1373 | (GGGAG) | 20(C8/9) | St3BE3 |
| | | AGGACCUAUAUCAGGGUGGG | 1374 | (GAG) | 20(C6/7) | SpBE3 |
| | | GUUUAGGACCUAUAUCAGGG | 1375 | (TGGGG) | 20(C10/11) | St3BE3 |
| | | UAGGUUUAGGACCUAUAUCA | 1376 | (GGGTG) | 20(C13/14) | St3BE3 |
| donor, | CTGTTTCACAGATGgtaagacaa | CCAUCUGUGAAACAGGCCUC | 1377 | (TGG) | 20(C1/2) | SpBE3 |
| intron 18 | (SEQ ID NO: 1488) | | | | | |
| | | UGUCUUACCAUCUGUGAAAC | 1378 | (AGG) | 20(C8/9) | SpBE3 |
| | | UUGUCUUACCAUCUGUGAAA | 1379 | (CAG) | 20(C9/10) | SpBE3 |
| acceptor, | gtctttcttgtcagGTTGTGTATG | CAUACACAACCUGACAAGAA | 1380 | (AGAC) | 20(C10/11) | VQR-SpBE3 |
| intron 18 | (SEQ ID NO: 1489) | | | | | |
| | | CCAUACACAACCUGACAAGA | 1381 | (AAG) | 20(C11/12) | SpBE3 |
| | | AACCUCCAUACACAACCUGA | 1382 | (CAAGAAA) | 20(C16/17) | St1BE3 |
| donor, | CTCAGCAGTGGTGCCCTGgtaaat | CCAGGGCACCACUGCUGAGC | 1383 | (AGG) | 20(C1/2) | SpBE3 |
| intron 19 | (SEQ ID NO: 1490) | | | | | |
| | | ACCAGGGCACCACUGCUGAG | 1384 | (CAG) | 20(C2/3) | SpBE3 |
| | | UUUACCAGGGCACCACUGCU | 1385 | (GAG) | 20(C5/6) | SpBE3 |
| | | AUUUACCAGGGCACCACUGC | 1386 | (TGAG) | 20(C6/7) | EQR-SpBE3 |
| | | ACCAGGGCACCACUGCUGAG | 1387 | (CAGGAT) | 20(C2/3) | SaBE3 |
| acceptor, | attatttccacagGCTTTTGAAGATA | AGCCUGUGGAAAUAAUAUUC | 1388 | (AAG) | 20(C3/4) | SpBE3 |
| intron 19 | (SEQ ID NO: 1491) | | | | | |
| | | AAAGCCUGUGGAAAUAAUAU | 1389 | (TCAAGT) | 20(C5/6) | KKH-SaBE3 |
| | | UAUCUUCAAAAGCCUGUGGA | 1390 | (AATAAT) | 20(C13/14) | KKH-SaBE3 |
| donor, | CCTAATTGTTGATgtaggtactt | ACAUCAACAAUUAGGAAAUC | 1391 | (CAG) | 20(C2) | SpBE3 |
| intron 20 | (SEQ ID NO: 1492) | | | | | |
| | | AGUACCUACAUCAACAAUUA | 1392 | (GGAA) | 20(C9) | VQR-SpBE3 |
| | | AAGUACCUACAUCAACAAUU | 1393 | (AGG) | 20(C10) | SpBE3 |
| | | AAAGUACCUACAUCAACAAU | 1394 | (TAG) | 20(C11) | SpBE3 |
| | | AGUACCUACAUCAACAAUUA | 1395 | (GGAAAT) | 20(C9) | KKH-SaBE3 |
| donor, | ATTTGAAGGAATGAGGgtaagaaaat | ACCCUCAUUCCUUCAAAUCU | 1396 | (AGAT) | 20(C2/3) | VQR-SpBE3 |
| intron 21 | (SEQ ID NO: 1493) | | | | | |
| | | UACCCUCAUUCCUUCAAAUC | 1397 | (TAG) | 20(C3/4) | SpBE3 |
| | | UUACCCUCAUUCCUUCAAAU | 1398 | (CTAGAT) | 20(C4/5) | KKH-SaBE3 |
| | | AUUUUCUUACCCUCAUUCCU | 1399 | (TCAAAT) | 20(C10/11) | KKH-SaBE3 |
| acceptor, | cttttgaatactagGTCGTTGTG | CUAGUAUUCAAAAGAAAGAA | 1400 | (AAG) | 20(C1) | SpBE3 |
| intron 21 | (SEQ ID NO: 1494) | | | | | |
| | | CGACCUAGUAUUCAAAAGAA | 1401 | (AGAA) | 20(C5) | VQR-SpBE3 |
| | | ACGACCUAGUAUUCAAAAGA | 1402 | (AAG) | 20(C6) | SpBE3 |
| | | ACAACGACCUAGUAUUCAAA | 1403 | (AGAA) | 20(C9) | VQR-SpBE3 |
| | | CACAACGACCUAGUAUUCAA | 1404 | (AAG) | 20(C10) | SpBE3 |
| | | AACGACCUAGUAUUCAAAAG | 1405 | (AAAGAAA) | 20(C7) | St1BE3 |
| | | UCACAACGACCUAGUAUUCA | 1406 | (AAAGAAA) | 20(C11) | St1BE3 |
| donor, | CTGCTTCAAGTTgtaagtgtccc | UUACAACUUGAAGCAGAGAU | 1407 | (AGG) | 20(C4) | SpBE3 |
| intron 22 | (SEQ ID NO: 1495) | | | | | |
| | | CUUACAACUUGAAGCAGAGA | 1408 | (TAG) | 20(C5) | SpBE3 |
| | | ACACUUACAACUUGAAGCAG | 1409 | (AGAT) | 20(C8) | VQR-SpBE3 |
| | | GACACUUACAACUUGAAGCA | 1410 | (GAG) | 20(C9) | SpBE3 |
| | | GGACACUUACAACUUGAAGC | 1411 | (AGAG) | 20(C9) | EQR-SpBE3 |
| | | GGGACACUUACAACUUGAAG | 1412 | (CAG) | 20(C11) | SpBE3 |
| | | ACUUACAACUUGAAGCAGAG | 1413 | (ATAGGT) | 20(C6) | KKH-SaBE3 |
| | | GGACACUUACAACUUGAAGC | 1414 | (AGAGAT) | 20(C10) | KKH-SaBE3 |
| acceptor, | attaatgttattcttaaagGCAACTT | CCUUUAAGAAUAACAUUAAU | 1415 | (AGAA) | 20(C1/2) | VQR-SpBE3 |
| intron 22 | (SEQ ID NO: 1496) | | | | | |
| | | GCCUUUAAGAAUAACAUUAA | 1416 | (TAG) | 20(C2/3) | SpBE3 |
| | | GCCUUUAAGAAUAACAUUAA | 1417 | (TAGAAT) | 20(C2/3) | SaBE3 |
| | | AAGUUGCCUUUAAGAAUAAC | 1418 | (ATTAAT) | 20(C7/8) | KKH-SaBE3 |
| | | UGCCUUUAAGAAUAACAUUA | 1419 | (ATAGAAT) | 20(C3/4) | St1BE3 |
| donor, | ATTCTGTTAATgtaagtattgattat | AUAAUCAAUACUUACAUUAA | 1420 | (CAGAAT) | 20(C15) | SaBE3 |
| intron 23 | (SEQ ID NO: 1497) | | | | | |
| | | GAUAAUCAAUACUUACAUUA | 1421 | (ACAGAAT) | 20(C16) | St1BE3 |
| acceptor, | acttttgtaaattttatagGTAGACA | CCUAUAAAAUUUACAAAAGU | 1422 | (TAG) | 20(C1/2) | SpBE3 |
| intron 23 | (SEQ ID NO: 1498) | | | | | |
| | | UCUACCUAUAAAAUUUACAA | 1423 | (AAG) | 20(C5/6) | SpBE3 |
| | | UGUCUACCUAUAAAAUUUAC | 1424 | (AAAAGT) | 20(C7/8) | KKH-SaBE3 |
| donor, | ACCAACAGAAAAAGAAGataagtatt | UAUCUUCUUUUUCUGUUGGU | 1425 | (TGAA) | 20(C4) | VQR-SpBE3 |
| intron 24 | (SEQ ID NO: 1499) | | | | | |
| | | UACUUAUCUUCUUUUUCUGU | 1426 | (TGG) | 20(C8) | SpBE3 |
| | | UAUCUUCUUUUUCUGUUGGU | 1427 | (TGAAAT) | 20(C4) | KKH-SaBE3 |
| | | AAUACUUAUCUUCUUUUUCU | 1428 | (GTTGGT) | 20(C10) | KKH-SaBE3 |
| donor, | CTCGACCAGGGgtaaaaaaatata | UUUACCCCUGGUCGAGGAAU | 1429 | (TGG) | 20(C10/11) | SpBE3 |
| intron 25 | (SEQ ID NO: 1500) | | | | | |
| | | AUUUUUUUACCCCUGGUCGA | 1430 | (GGAA) | 20(C9/10) | VQR-SpBE3 |
| | | UAUUUUUUUACCCCUGGUCG | 1431 | (AGG) | 20(C5/6) | SpBE3 |
| | | AUAUUUUUUUACCCCUGGUC | 1432 | (GAG) | 20(C10/11) | SpBE3 |
| | | UAUAUUUUUUUACCCCUGGU | 1433 | (CGAG) | 20(C11/12) | EQR-SpBE3 |
| | | UAUUUUUUUACCCCUGGUCG | 1434 | (AGGAAT) | 20(C12/13) | SaBE3 |
| acceptor, | cttatttctttgcagAACAAAAT | UUUUGUUCUGCAAAGAAAUA | 1435 | (AGAA) | 20(C13/14) | VQR-SpBE3 |
| intron 25 | (SEQ ID NO: 1501) | | | | | |
| | | AUUUUGUUCUGCAAAGAAAU | 1436 | (AAG) | 20(C11/12) | SpBE3 |
| | | UUGUUCUGCAAAGAAAUAAG | 1437 | (AATAAT) | 20(C8) | KKH-SaBE3 |
| | | AUUUUGUUCUGCAAAGAAAU | 1438 | (AAGAAT) | 20(C9) | SaBE3 |
| | | CCUUGGAUUUUGUUCUGCAA | 1439 | (AGAAAT) | 20(C6) | KKH-SaBE3 |
| | | GAUUUUGUUCUGCAAAGAAA | 1440 | (TAAGAAT) | 20(C9) | St1BE3 |
| donor, | CTCCATTGTAGgtaagaatattt | AAUAUUCUUACCUACAAUGG | 1441 | (AGAT) | 20(C15) | VQR-SpBE3 |
| intron 26 | (SEQ ID NO: 1502) | | | | | |
| | | AAAUAUUCUUACCUACAAUG | 1442 | (GAG) | 20(C10) | SpBE3 |
| | | UAAAUAUUCUUACCUACAAU | 1443 | (GGAG) | 20(C11/12) | EQR-SpBE3 |
| | | AUAUUCUUACCUACAAUGGA | 1444 | (GATAAT) | 20(C12/13) | KKH-SaBE3 |
| | | UAAAUAUUCUUACCUACAAU | 1445 | (GGAGAT) | 20(C13/14) | KKH-SaBE3 |
| | | AUAAAUAUUCUUACCUACAA | 1446 | (TGGAG) | 20(C10/11) | St3BE3 |
| acceptor, | ctccacatacagGTATGTTTCTAG | CUGUAUGUGGAGGAAAAUAA | 1447 | (TAG) | 20(C13/14) | SpBE3 |
| intron 26 | (SEQ ID NO: 1503) | | | | | |
| | | GAAACAUACCUGUAUGUGGA | 1448 | (GGAA) | 20(C14/15) | VQR-SpBE3 |
| | | AGAAACAUACCUGUAUGUGG | 1449 | (AGG) | 20(C1) | SpBE3 |
| | | UAGAAACAUACCUGUAUGUG | 1450 | (GAG) | 20(C10) | SpBE3 |
| | | CUAGAAACAUACCUGUAUGU | 1451 | (GGAG) | 20(C11) | EQR-SpBE3 |
| | | CAUACCUGUAUGUGGAGGAA | 1452 | (AATAAT) | 20(C12) | KKH-SaBE3 |
| | | AAACAUACCUGUAUGUGGAG | 1453 | (GAAAAT) | 20(C13) | KKH-SaBE3 |
| | | CCUGUAUGUGGAGGAAAAUA | 1454 | (ATAGAAA) | 20(C6) | St1BE3 |
| | | GCUAGAAACAUACCUGUAUG | 1455 | (TGGAG) | 20(C9) | St3BE3 |
| | | CUAGAAACAUACCUGUAUGU | 1456 | (GGAG) | 20(C2) | EQR-SpBE3 |

^{a}BE types: SpBE3 = APOBEC1-SpCas9n-UGI; VQR-SpBE3 = APOBEC1-VQR-SpCas9n-UGI; EQR-SpBE3 = APOBEC1-EQR-SpCas9n-UGI; VRER-SpBE3 = APOBEC1-VRER-SpCas9n-UGI; SaBE3 = APOBEC1-SaCas9n-UGI; KKH-SaBE3 = APOBEC1-KKH-SaCas9n-UGI; St3BE3 = APOBEC1-St3Cas9n-UGI; St1BE3 = APOBEC1-St1Cas9n-UGI.
*Isoform 2 is expressed preferentially in the dorsal root ganglion.

Scoring of Guide RNA Sequences for Efficient Base Editing with High Specificity and Low Off-Target Binding

To achieve efficient and specific genome modifications using base editing requires judicious selection of a genomic sequence containing a target C, for which a specific complementary guide RNA sequence can be generated, and if required, a nearby PAM that matches the DNA-binding domain that is fused to the cytidine deaminase (e.g. Cas9, dCas9, Cas9n, Cpf1, NgAgo, etc.), as described in Komor et al., Nature, 533, 420-424 (2016), which is incorporated herein by reference. The guide RNA sequence and PAM preference define the genomic target sequence(s) of programmable DNA-binding domains (e.g. Cas9, dCas9, Cas9n, Cpf1, NgAgo, etc.). Because of the repetitive nature of some genomic sequences as well as the stochastic frequency of representation of short sequences throughout the genome it is necessary to identify guide RNAs for programming base editors that have the lowest number of potential off target sites, taking into consideration 1, 2, 3, 4, or more mismatches against all other sequences in the genome as described in Hsu et al (Nature Biotechnology, 2013, 31(9):827-832), Fusi et al. (bioRxiv 021568; doi: http://dx.doi.org/10.1101/021568), Chari et al. (Nature Methods, 2015, 12(9):823-6), Doench et al. (Nature Biotechnology, 2014, 32(12):1262-7), Wang et al. (Science. 2014, 343(6166): 80-4). Moreno-Mateos et al (Nature Methods, 2015, 12(10):982-8), Housden et al. (Science Signaling, 2015, 8(393):rs9), Haeussler et al., (Genome Biol. 2016; 17: 148), each of which is incorporated herein by reference. The potential for the formation of bulges between the guide RNA and the target DNA may also be considered as described in Bae et al. (Bioinformatics, 2014, 30, 1473-5), which is incorporated herein by reference. Non-limiting examples of calculated specificity scores for selected guide RNAs are shown in Tables 7-9. Other calculated parameters that may influence DNA-binding domains programming efficiency are shown, as described in Housden et al. (Science Signaling, 2015, 8(393):rs9), Farboud et al. (Genetics, 2015, 199(4):959-71), each of which is incorporated herein by reference.

TABLE 7
Exemplary Efficiency and Specificity Scores for gRNAs for NaV1.7 (SCN9A) Protective Loss-of-Function Mutations via
Premature Stop Codons

| | | Programmable | SEQ | | | | | | | | | | | Pro | |
|---|---|---|---|---|---|---|---|---|---|---|---|---|---|---|---|
| Target | BE | guide-RNA | ID | | gRNA size | | | | | | | M.- | Hous- | x/ | Off |
| variants | type^{a} | sequence | NOs | PAM | (C edited) | Eff^{b} | Hsu^{c} | Fusi | Chari | Doench | Wang | M. | den | GC | targets^{d} |
| Q687X | EQR- | CAACCUCAGACAG | 1504 | (TGAG) | 20 (C7) | 5.4 | 99 | 62 | 92 | 19 | 80 | 35 | 5 | − | 0-0-0-0-8 |
| | SpBE3 | AGAGCAA | | | | | | | | | | | | | |
| Q687X | KKH- | GAUCCCAACCUCA | 1505 | (AGCAAT) | 20 (C12) | 6.2 | 92 | 66 | 99 | 39 | 77 | 26 | 6 | − | 0-0-0-2-13 |
| | SaBE3 | GACAGAG | | | | | | | | | | | | | |
| W1245X | KKH- | AAAUCCAGCCAAC | 1506 | (ATTGGT) | 20 (C14/8) | 6.6 | 96 | 50 | 95 | 10 | 84 | 36 | 6 | + | 0-0-0-0-8 |
| | SaBE3 | ACCAGGC | | | | | | | | | | | | | |
| Q323X | SaBE3 | CGUGUGUAGUCAG | 1507 | (AGGGGT) | 20 (C11) | 8.2 | 96 | 60 | 93 | 36 | 78 | 69 | 8 | + | 0-0-0-2-5 |
| | | UGUCCAG | | | | | | | | | | | | | |
| Q323X | St3BE3 | CGUGUGUAGUCAG | 1508 | (AGGGG) | 20 (C11) | 8.2 | 96 | 60 | 93 | 36 | 78 | 69 | 8 | + | 0-0-0-1-16 |
| | | UGUCCAG | | | | | | | | | | | | | |
| W188X | SpBE3 | GUUCCACGGGUCA | 1509 | (AAG) | 20 (C5) | 5.3 | 95 | 51 | 92 | 13 | 55 | 51 | 5 | − | 0-0-0-2-45 |
| | | CGAAGAA | | | | | | | | | | | | | |
| Q1494X | SpBE3 | AAAGCCAAUUCCU | 1510 | (GGG) | 20 (C-2) | 4.9 | 88 | 68 | 96 | 40 | 84 | 64 | 4 | + | 0-0-1-9-68 |
| | | CGACCAG | | | | | | | | | | | | | |
| R835X | KKH- | UCAGUUCUGCGAU | 1511 | (ACTGGT) | 20 (C10) | 6.8 | 98 | 60 | 85 | 51 | 71 | 58 | 6 | − | 0-0-0-0-5 |
| | SaBE3 | CAUUCAG | | | | | | | | | | | | | |
| R841X | KKH- | GCUCCGAGUCUUC | 1512 | (CAAAAT) | 20 (C5) | 6.6 | 98 | 51 | 84 | 63 | 66 | 58 | 6 | − | 0-0-0-1-3 |
| | SaBE3 | AAGUUGG | | | | | | | | | | | | | |
| Q485X | St3BE3 | AAUCAAAAGAAGC | 1513 | (TGGAG) | 20 (C4) | 7.5 | 94 | 61 | 87 | 24 | 85 | 50 | 7 | + | 0-0-0-1-38 |
| | | UCUCCAG | | | | | | | | | | | | | |
| Q643X | KKH- | AAUGGACAGCUUC | 1514 | (GGTGAT) | 20 (C7) | 9.9 | 95 | 60 | 85 | 65 | 67 | 54 | 9 | + | 0-0-0-2-8 |
| | SaBE3 | UGCCAGA | | | | | | | | | | | | | |
| W730X | KKH- | GAAUUUUAUCCAA | 1515 | (AGCAAT) | 20 (C11) | 6.6 | 94 | 60 | 84 | 20 | 88 | 21 | 6 | − | 0-0-0-1-17 |
| | SaBE3 | UAUGGAG | | | | | | | | | | | | | |
| Q1862X | KKH- | UCUUCGUUCACAG | 1516 | (AAAGGT) | 20 (C11) | 4.8 | 92 | 54 | 85 | 40 | 70 | 32 | 4 | − | 0-0-0-2- |
| | SaBE3 | AUGGAAG | | | | | | | | | | | | | 44 |
| Q595X | St3BE3 | GUUUGUGCCCCAC | 1517 | (AGGAG) | 20 (C13) | 7.0 | 90 | 55 | 86 | 45 | 74 | 38 | 7 | + | 0-0-0-3- |
| | | AGACCCC | | | | | | | | | | | | | 38 |
| W151X | SpBE3 | ACAUUUUUGGUCC | 1518 | (TGG) | 20 (C13) | 4.9 | 87 | 51 | 88 | 39 | 84 | 46 | 4 | + | 0-0-1-5- |
| | | AGUCCGG | | | | | | | | | | | | | 85 |
| R523X | SpBE3 | AUAGGCGAGCACA | 1519 | (AGG) | 20 (C6) | 10.6 | 78 | 61 | 96 | 75 | 78 | 58 | 10 | − | 0-0-0-5- |
| | | UGAAAAG | | | | | | | | | | | | | 86 |
| Q534X | KKH- | UACCCCCAAUCAG | 1520 | (CCAAAT) | 20 (C11) | 4.7 | 96 | 56 | 77 | 5 | 47 | 48 | 4 | − | 0-0-0-0- |
| | SaBE3 | GUACCAC | | | | | | | | | | | | | 3 |
| W714X | SpBE3 | GCAAAUCUGUACC | 1521 | (TGG) | 20 (C13) | 5.2 | 73 | 71 | 99 | 59 | 85 | 57 | 5 | + | 0-0-1- |
| | | ACCAAGG | | | | | | | | | | | | | 14-113 |
| Q1494X | SpBE3 | AAAAGCCAAUUCC | 1522 | (GGG) | 20 (C-1) | 6.0 | 87 | 61 | 85 | 14 | 64 | 56 | 6 | + | 0-0-2- |
| | | UCGACCA | | | | | | | | | | | | | 12-86 |
| W188X | SaBE3 | UCCACGGGUCACG | 1523 | (GTGAAT) | 20 (C3) | 6.9 | 96 | 56 | 75 | 55 | 51 | 57 | 6 | − | 0-0-0-0- |
| | | AAGAAAA | | | | | | | | | | | | | 4 |
| W1245X | KKH- | CAGCCAACACCAG | 1524 | (TGAAAT) | 20 (C9/3) | 7.0 | 91 | 50 | 80 | 9 | 60 | 48 | 7 | − | 0-0-0-1- |
| | SaBE3 | GCAUUGG | | | | | | | | | | | | | 16 |
| W188X | KKH- | CAGUUCCACGGGU | 1525 | (AAAAGT) | 20 (C7/1) | 4.3 | 99 | -1 | 71 | 22 | 66 | 65 | 4 | − | 0-0-0-0- |
| | SaBE3 | CACGAAG | | | | | | | | | | | | | 14 |
| Q595X | SpBE3 | CACAGACCCCAGG | 1526 | (CAG) | 20 (C3) | 7.7 | 75 | 61 | 95 | 65 | 87 | 69 | 5 | + | 0-0-4- |
| | | AGCGACG | | | | | | | | | | | | | 22-140 |
| Q1004X | VQR- | UAUGUGAAACAAA | 1527 | (TGAA) | 20 (C10) | 7.2 | 80 | 60 | 90 | 35 | 71 | 16 | 7 | - | 0-0-1- |
| | SpBE3 | CCUUACG | | | | | | | | | | | | | 10-146 |
| W1578X | KKH- | AAAAAUAUUCCAU | 1528 | (TGAAGT) | 20 (C11) | 5.2 | 86 | 62 | 83 | 13 | 81 | 35 | 5 | - | 0-0-0-4- |
| | SaBE3 | CCUACAG | | | | | | | | | | | | | 22 |
| Q368/9X | St1BE3 | UUACCAACAGGUG | 1529 | (AGAGAAA) | 20 (C5) | 4.3 | 98 | 66 | 70 | 50 | 59 | 38 | 4 | - | 0-0-0-0- |
| | | AGUACCA | | | | | | | | | | | | | 18 |
| Q369X | St1BE3 | UUACCAACAGGUG | 1530 | (AGAGAAA) | 20 (C8) | 4.3 | 98 | 66 | 70 | 50 | 59 | 38 | 4 | - | 0-0-0-0- |
| | | AGUACCA | | | | | | | | | | | | | 18 |
| W188X | VQR- | GCCAGUUCCACGG | 1531 | (AGAA) | 20 (C9/3) | 2.9 | 91 | 63 | 75 | 38 | 77 | 46 | 2 | - | 0-0-3-0- |
| | SpBE3 | GUCACGA | | | | | | | | | | | | | 33 |
| W151X | SaBE3 | GACAUUUUUGGUC | 1532 | (GTGGGT) | 20 (C14) | 3.6 | 99 | 49 | 66 | 27 | 69 | 52 | 3 | + | 0-0-0-0- |
| | | CAGUCCG | | | | | | | | | | | | | 4 |
| W1332X | KKH- | UCAGCCAGAAUAU | 1533 | (ACAAGT) | 20 (C6) | 3.6 | 92 | 67 | 73 | 51 | 78 | 51 | 3 | - | 0-0-0-2- |
| | SaBE3 | AAGACAC | | | | | | | | | | | | | 18 |
| W908X | KKH- | CAUGUGCCACCGU | 1534 | (TACAGT) | 20 (C8) | 6.3 | 92 | 59 | 72 | 5 | 58 | 56 | 6 | + | 0-0-0-2- |
| | SaBE3 | GGGAGCG | | | | | | | | | | | | | 8 |
| Q534X | KKH- | AUCAGGUACCACC | 1535 | (CTAAAT) | 20 (C3) | 6.2 | 99 | 59 | 63 | 16 | 64 | 42 | 6 | - | 0-0-0-0- |
| | SaBE3 | CAAAUUG | | | | | | | | | | | | | 5 |
| Q1004X | SaBE3 | UUAUGUGAAACAA | 1536 | (GTGAAT) | 20 (C11) | 4.8 | 96 | 44 | 65 | 13 | 28 | 37 | 4 | - | 0-0-0-2- |
| | | ACCUUAC | | | | | | | | | | | | | 21 |
| Q1907X | KKH- | ACCGCUUAAGGCA | 1537 | (AAAAAT) | 20 (C12) | 3.9 | 98 | 49 | 62 | 3 | 35 | 44 | 3 | - | 0-0-0-1- |
| | SaBE3 | AAAUGUC | | | | | | | | | | | | | 3 |
| Q663X | St1BE3 | GGCACGACCAAUC | 1538 | (CAAGAAA) | 20 (C9) | 4.4 | 96 | 63 | 58 | 29 | 72 | 27 | 4 | - | 0-0-0-1- |
| | | AAAUACA | | | | | | | | | | | | | 13 |
| R1381X | KKH- | AAUGUGCGAUGGA | 1539 | (GAAAGT) | 20 (C7) | 4.0 | 91 | 67 | 68 | 49 | 81 | 64 | 4 | - | 0-0-0-2- |
| | SaBE3 | AAAACCU | | | | | | | | | | | | | 17 |
| R1619X | St3BE3 | CGAAUCCUACGUC | 1540 | (AGGAG) | 20 (C1) | 8.5 | 99 | 60 | 54 | 32 | 49 | 54 | 8 | - | 0-0-3-0- |
| | | UAGUCAA | | | | | | | | | | | | | 0 |
| Q58X | St3BE3 | AAACAGCUGCCCU | 1541 | (TGGGG) | 20 (C4) | 8.4 | 96 | 26 | 61 | 3 | 53 | 35 | 8 | - | 0-0-0-3- |
| | | UCAUCUA | | | | | | | | | | | | | 29 |
| Q708X | St3BE3 | CCAGACAAAAAUG | 1542 | (TGGTG) | 20 (C6) | 7.2 | 90 | 67 | 55 | 13 | 73 | 47 | 7 | + | 0-0-1-4- |
| | | UCCACCU | | | | | | | | | | | | | 35 |
| Q25X | St1BE3 | CAUUGAACAACGC | 1543 | (AAAGAAA) | 20 (C8) | 3.8 | 97 | 59 | 59 | 27 | 79 | 26 | 3 | - | 0-0-0-0- |
| | | AUUGCUG | | | | | | | | | | | | | 16 |
| Q1971X | St1BE3 | AGAAAUAUGAACA | 1544 | (ACAGAAA) | 20 (C12) | 5.8 | 64 | 53 | 92 | 13 | 80 | 17 | 5 | - | 0-0-0- |
| | | AGACAGA | | | | | | | | | | | | | 20-242 |
| Q240X | St1BE3 | GGGGCUUUGAUCC | 1545 | (GAAGAAG) | 20 (C13) | 5.4 | 95 | 60 | 50 | 8 | 62 | 48 | 5 | - | 0-0-1-3- |
| | | AGUCAGU | | | | | | | | | | | | | 11 |
| Q595X | KKH- | ACAGACCCCAGGA | 1546 | (AGCAGT) | 20 (C2) | 4.3 | 97 | 52 | 58 | 14 | 77 | 55 | 4 | + | 0-0-0-2- |
| | SaBE3 | GCGACGC | | | | | | | | | | | | | 12 |
| R597X | SpBE3 | GAGCGACGCAGCA | 1547 | (CAG) | 20 (C4) | 4.1 | 91 | 64 | 58 | 48 | 76 | 63 | 4 | - | 0-0-1-0- |
| | | GUAACAU | | | | | | | | | | | | | 43 |
| R1619X | SpBE3 | CGAAUCCUACGUC | 1548 | (AGG) | 20 (C1) | 8.5 | 95 | 60 | 54 | 32 | 49 | 54 | 8 | - | 0-0-3-1- |
| | | UAGUCAA | | | | | | | | | | | | | 12 |
| R1619X | EQR- | GAAUCCUACGUCU | 1549 | (GGAG) | 20 (C-1) | 4.1 | 78 | 47 | 76 | 19 | 45 | 27 | 4 | - | 0-0-3-9- |
| | SpBE3 | AGUCAAA | | | | | | | | | | | | | 62 |
| Q663X | SpBE3 | GCACGACCAAUCA | 1550 | (AAG) | 20 (C8) | 3.3 | 86 | 67 | 54 | 68 | 77 | 37 | 3 | - | 0-0-0-3- |
| | | AAUACAC | | | | | | | | | | | | | 36 |
| Q1539/ | KKH- | GGUCAAAGUCAAC | 1551 | (TGAAGT) | 20 (C4/10) | 6.7 | 90 | 56 | 63 | 22 | 57 | 14 | 6 | - | 0-0-1-1- |
| 41X | SaBE3 | AUAUGAC | | | | | | | | | | | | | 6 |
| Q604X | SpBE3 | GUAACAUCAGCCA | 1552 | (AGG) | 20 (C12) | 3.8 | 68 | 61 | 84 | 76 | 61 | 33 | 3 | + | 0-0-2- |
| | | AGCCAGU | | | | | | | | | | | | | 14-105 |
| W1161X | SpBE3 | ACCUCCAUACACA | 1553 | (AAG) | 20 (C6) | 7.5 | 82 | 54 | 70 | 28 | 27 | 35 | 7 | + | 0-0-2-5- |
| | | ACCUGAC | | | | | | | | | | | | | 85 |
| Q1378X | SpBE3 | AUGUUAGUCAAAA | 1554 | (TGG) | 20 (C9) | 6.6 | 87 | 65 | 46 | 53 | 89 | 30 | 6 | + | 0-0-1-7- |
| | | UGUGCGA | | | | | | | | | | | | | 78 |
| W1786X | KKH- | CCAAACCUCAUAG | 1555 | (CAAAGT) | 20 (C2) | 4.4 | 91 | 61 | 52 | 20 | 51 | 34 | 4 | - | 0-0-0-4- |
| | SaBE3 | AACAUCU | | | | | | | | | | | | | 16 |
| R277X | KKH- | GUUUUCGAAAUUC | 1556 | (AATAAT) | 20 (C6) | 5.3 | 90 | 50 | 61 | 8 | 46 | 37 | 5 | - | 0-0-0-6- |
| | SaBE3 | ACUUGAA | | | | | | | | | | | | | 48 |
| Q604X | KKH- | AGCCAAGCCAGUA | 1557 | (ACCAAT) | 20 (C4) | 4.8 | 97 | 54 | 25 | 7 | 48 | 38 | 4 | + | 0-0-0-0- |
| | SaBE3 | GGUCCCC | | | | | | | | | | | | | 8 |
| Q643X | St3BE3 | CAAUGGACAGCUU | 1558 | (AGGTG) | 20 (C8) | 4.2 | 90 | 61 | 61 | 15 | 67 | 54 | 4 | + | 0-0-0-5- |
| | | CUGCCAG | | | | | | | | | | | | | 32 |
| W151X | SpBE3 | CAUUUUUGGUCCA | 1559 | (GGG) | 20 (C12) | 4.8 | 94 | 56 | 46 | 36 | 38 | 48 | 4 | + | 0-0-0-1- |
| | | GUCCGGU | | | | | | | | | | | | | 48 |
| W188X | St1BE3 | CAGCCAGUUCCAC | 1560 | (GAAGAAA) | 20 (C11/5) | 6.9 | 98 | 42 | 52 | 1 | 27 | 59 | 6 | + | 0-0-1-0- |
| | | GGGUCAC | | | | | | | | | | | | | 11 |
| Q687X | SaBE3 | CCAACCUCAGACA | 1561 | (ATGAGT) | 20 (C8) | 3.7 | 86 | 59 | 64 | 38 | 61 | 44 | 3 | - | 0-0-0-3- |
| | | GAGAGCA | | | | | | | | | | | | | 19 |
| Q1363X | SaBE3 | AAGUCAAGUUCCA | 1562 | (CCGAAT) | 20 (C5) | 4.1 | 99 | 51 | 28 | 26 | 49 | 58 | 4 | - | 0-0-0-0- |
| | | AAUCGUU | | | | | | | | | | | | | 7 |
| Q1378X | VQR- | UGUUAGUCAAAAU | 1563 | (GGAA) | 20 (C8) | 4.3 | 89 | 61 | 15 | 9 | 41 | 48 | 4 | - | 0-0-0-6- |
| | SpBE3 | GUGCGAU | | | | | | | | | | | | | 85 |
| Q1515X | KKH- | ACCUAGUGACAAA | 1564 | (TTTGAT) | 20 (C10) | 7.6 | 93 | 47 | 57 | 11 | 60 | 1 | 7 | + | 0-0-0-1- |
| | SaBE3 | UCAAGCC | | | | | | | | | | | | | 9 |
| R1499X | KKH- | AAUUCCUCGACCA | 1565 | (AAAAAT) | 20 (C8) | 5.6 | 99 | 50 | 33 | 10 | 43 | 54 | 5 | - | 0-0-0-0- |
| | SaBE3 | GGGGUAA | | | | | | | | | | | | | 2 |
| Q643X | KKH- | cCCAAUGGACAGC | 1566 | (AGAGGT) | 20 (C10) | 7.9 | 88 | 36 | 60 | 11 | 35 | 34 | 7 | + | 0-0-2-1- |
| | SaBE3 | UUCUGCC | | | | | | | | | | | | | 10 |
| Q989X | St1BE3 | CAACCUCCAGAUU | 1567 | (CTAGAAT) | 20 (C8) | 4.5 | 93 | 54 | 21 | 6 | 65 | 41 | 4 | - | 0-0-1-2- |
| | | GCAGUGA | | | | | | | | | | | | | 15 |
| Q1167X | SaBE3 | CUCAUGCUGCCAA | 1568 | (TAGAGT) | 20 (C11) | 6.1 | 93 | 54 | 46 | 11 | 31 | 14 | 6 | - | 0-0-0-0- |
| | | GUUAACA | | | | | | | | | | | | | 19 |
| W1408X | KKH- | AUAAUAAUCGUCC | 1569 | (AAAAGT) | 20 (C13) | 4.1 | 94 | 51 | 53 | 62 | 46 | 41 | 4 | - | 0-0-0-0- |
| | SaBE3 | AUCCCUU | | | | | | | | | | | | | 20 |
| Q58X | SpBE3 | ACAGCUGCCCUUC | 1570 | (GGG) | 20 (C2) | 6.7 | 69 | 71 | 77 | 39 | 52 | 44 | 6 | - | 0-0-4- |
| | | AUCUAUG | | | | | | | | | | | | | 19-191 |
| R523X | SpBE3 | GGCAUAGGCGAGC | 1571 | (AAG) | 20 (C9) | 5.0 | 69 | 57 | 77 | 23 | 58 | 51 | 5 | - | 0-0-2- |
| | | ACAUGAA | | | | | | | | | | | | | 14-83 |
| R548X | KKH- | CUGCAAGGCGAAG | 1572 | (ACAAGT) | 20 (C9) | 5.2 | 74 | 56 | 72 | 35 | 65 | 75 | 5 | - | 0-0-1-2- |
| | SaBE3 | CAGCAGA | | | | | | | | | | | | | 27 |
| Q663X | St3BE3 | CCAAUCAAAUACA | 1573 | (AGGCG) | 20 (C2) | 4.5 | 82 | 47 | 64 | 11 | 71 | 28 | 4 | - | 0-0-0-7- |
| | | CAAGAAA | | | | | | | | | | | | | 63 |
| W1700X | KKH- | CCAUCCCAGCCAG | 1574 | (TGTAAT) | 20 (C7) | 7.3 | 79 | 54 | 67 | 10 | 58 | 35 | 7 | - | 0-0-0-2- |
| | SaBE3 | CAGAGGU | | | | | | | | | | | | | 35 |
| R523X | KKH- | GCAUAGGCGAGCA | 1575 | (AGAGGT) | 20 (C8) | 4.3 | 92 | 48 | 53 | 23 | 83 | 41 | 4 | - | 0-0-0-2- |
| | SaBE3 | CAUGAAA | | | | | | | | | | | | | 9 |
| R835X | SpBE3 | AGUUCUGCGAUCA | 1576 | (TGG) | 20 (C8) | 7.1 | 81 | 64 | 33 | 20 | 51 | 32 | 7 | - | 0-0-1-5- |
| | | UUCAGAC | | | | | | | | | | | | | 42 |
| R548X | KKH- | GAAGCAGCAGAAC | 1577 | (TTTAGT) | 20 (C-1) | 4.4 | 86 | 39 | 58 | 24 | 69 | 52 | 4 | - | 0-0-0-4- |
| | SaBE3 | AAGUCUU | | | | | | | | | | | | | 12 |
| Q360X | SpBE3 | GCUAAUGACCCAA | 1578 | (GGG) | 20 (C11) | 6.0 | 71 | 55 | 72 | 25 | 36 | 15 | 6 | - | 0-0-3-8- |
| | | GAUUACU | | | | | | | | | | | | | 74 |
| Q643X | KKH- | GGACAGCUUCUGC | 1579 | (GATAAT) | 20 (C4) | 5.1 | 81 | 62 | 40 | 20 | 75 | 46 | 5 | - | 0-0-0-5- |
| | SaBE3 | CAGAGGU | | | | | | | | | | | | | 14 |
| R1381X | VQR- | GUGCGAUGGAAAA | 1580 | (AGTG) | 20 (C4) | 5.5 | 59 | 58 | 84 | 4 | 59 | 48 | 5 | - | 0-0-1- |
| | SpBE3 | ACCUGAA | | | | | | | | | | | | | 21-169 |
| W1578X | KKH- | AAUAUUCCAUCCU | 1581 | (AGTAGT) | 20 (C8) | 4.1 | 83 | 60 | 44 | 13 | 74 | 43 | 4 | - | 0-0-2-3- |
| | SaBE3 | ACAGUGA | | | | | | | | | | | | | 37 |
| Q25X | VQR- | UUGAACAACGCAU | 1582 | (AGAA) | 20 (C6) | 6.0 | 54 | 59 | 88 | 16 | 31 | 43 | 6 | - | 0-0-1- |
| | SpBE3 | UGCUGAA | | | | | | | | | | | | | 31-326 |
| Q368/9X | EQR- | UUACCAACAGGUG | 1583 | (AGAG) | 20 (C5) | 4.3 | 72 | 66 | 70 | 50 | 59 | 38 | 4 | - | 0-0-1- |
| | SpBE3 | AGUACCA | | | | | | | | | | | | | 13-111 |
| Q369X | EQR- | UUACCAACAGGUG | 1584 | (AGAG) | 20 (C8) | 4.3 | 72 | 66 | 70 | 50 | 59 | 38 | 4 | - | 0-0-1- |
| | SpBE3 | AGUACCA | | | | | | | | | | | | | 13-111 |
| Q941X | VQR- | GGAGGUCGCUGGU | 1585 | (TGTG) | 20 (C14) | 3.8 | 91 | 43 | 51 | 15 | 82 | 53 | 3 | - | 0-0-0-2- |
| | SpBE3 | CAAGCUA | | | | | | | | | | | | | 44 |
| Q1167X | SpBE3 | GCCAAGUUAACAU | 1586 | (GGG) | 20 (C3) | 2.9 | 76 | 65 | 66 | 60 | 74 | 29 | 2 | - | 0-0-0- |
| | | AGAGUCA | | | | | | | | | | | | | 13-103 |
| Q989X | SaBE3 | AACCUCCAGAUUG | 1587 | (TAGAAT) | 20 (C7) | 6.3 | 92 | 49 | 11 | 3 | 24 | 44 | 6 | - | 0-0-1-1- |
| | | CAGUGAC | | | | | | | | | | | | | 9 |
| W1578X | KKH- | AUUCCAUCCUACA | 1588 | (AGTAGT) | 20 (C5) | 4.4 | 89 | 51 | 15 | 15 | 37 | 35 | 4 | - | 0-0-0-1- |
| | SaBE3 | GUGAAGU | | | | | | | | | | | | | 20 |
| Q708X | SpBE3 | GACAAAAAUGUCC | 1589 | (TGG) | 20 (C3) | 3.9 | 47 | 55 | 92 | 17 | 67 | 56 | 3 | + | 0-0-3- |
| | | ACCUUGG | | | | | | | | | | | | | 25-208 |
| Q708X | KKH- | GUCCAGACAAAAA | 1590 | (CTTGGT) | 20 (C8) | 6.0 | 76 | 54 | 63 | 28 | 71 | 34 | 6 | + | 0-0-0- |
| | SaBE3 | UGUCCAC | | | | | | | | | | | | | 12-58 |
| W724X | KKH- | UCCAGAUCAAGAA | 1591 | (GCAAAT) | 20 (C3) | 4.9 | 79 | 60 | 6 | 17 | 56 | 24 | 4 | - | 0-0-2-3- |
| | SaBE3 | UUUGUGU | | | | | | | | | | | | | 32 |
| Q805X | SaBE3 | AUGAGUAUUUCCA | 1592 | (TGGAAT) | 20 (C12) | 4.4 | 88 | 51 | 39 | 7 | 51 | 32 | 4 | + | 0-0-1-4- |
| | | AGUAGGC | | | | | | | | | | | | | 12 |
| Q485X | EQR- | CAAAAGAAGCUCU | 1593 | (AGAG) | 20 (C1) | 6.8 | 62 | 57 | 75 | 5 | 83 | 50 | 6 | + | 0-0-2- |
| | SpBE3 | CCAGUGG | | | | | | | | | | | | | 19-210 |
| W1245X | St1BE3 | AUAACCAUAUGCU | 1594 | (TTAGAAG) | 20 (C17) | 4.5 | 89 | 41 | 48 | 51 | 23 | 22 | 4 | - | 0-0-2-3- |
| | | AUCCAUU | | | | | | | | | | | | | 17 |
| Q1505X | KKH- | AUCCAAGGAUGUA | 1595 | (CCTAGT) | 20 (C4) | 5.8 | 87 | 50 | 20 | 16 | 29 | 44 | 5 | - | 0-0-0-4- |
| | SaBE3 | UAUUUGA | | | | | | | | | | | | | 32 |
| Q1363X | VQR- | AGUCAAGUUCCAA | 1596 | (CGAA) | 20 (C4) | 6.2 | 88 | 48 | 19 | 32 | 44 | 29 | 6 | - | 0-0-1-4- |
| | SpBE3 | AUCGUUC | | | | | | | | | | | | | 92 |
| Q58X | SpBE3 | AAACAGCUGCCCU | 1597 | (TGG) | 20 (C4) | 8.4 | 74 | 26 | 61 | 3 | 53 | 35 | 8 | - | 0-0-2- |
| | | UCAUCUA | | | | | | | | | | | | | 12-147 |
| Q368/9X | SpBE3 | UUUACCAACAGGU | 1598 | (AAG) | 20 (C6) | 4.8 | 81 | 54 | 44 | 5 | 23 | 34 | 4 | - | 0-0-1-9- |
| | | GAGUACC | | | | | | | | | | | | | 88 |
| Q1401X | KKH- | AAGGCAACUUUUA | 1599 | (GACGAT) | 20 (C5) | 5.2 | 83 | 52 | 38 | 2 | 62 | 62 | 5 | + | 0-0-1-6- |
| | SaBE3 | AGGGAUG | | | | | | | | | | | | | 28 |
| Q1515X | KKH- | UGACAAAUCAAGC | 1600 | (ATTAGT) | 20 (C4) | 4.2 | 92 | 43 | 14 | 32 | 34 | 29 | 4 | - | 0-0-0-1- |
| | SaBE3 | CUUUGAU | | | | | | | | | | | | | 18 |
| Q643X | KKH- | AGCUUCUGCCAGA | 1601 | (ATAGAT) | 20 (C-1) | 6.7 | 94 | 40 | 24 | 1 | 30 | 23 | 6 | - | 0-0-0-3- |
| | SaBE3 | GGUGAUA | | | | | | | | | | | | | 16 |
| Q25X | KKH- | UGAACAACGCAUU | 1602 | (GAAAAT) | 20 (C5) | 7.6 | 88 | 45 | 12 | 7 | 47 | 41 | 7 | - | 0-0-0-3- |
| | SaBE3 | GCUGAAA | | | | | | | | | | | | | 14 |
| Q368/9X | SpBE3 | UACCAACAGGUGA | 1603 | (GAG) | 20 (C4) | 4.2 | 71 | 58 | 62 | 27 | 54 | 63 | 4 | - | 0-0-1- |
| | | GUACCAA | | | | | | | | | | | | | 10-123 |
| Q369X | SpBE3 | UACCAACAGGUGA | 1604 | (GAG) | 20 (C7) | 4.2 | 71 | 58 | 62 | 27 | 54 | 63 | 4 | - | 0-0-1- |
| | | GUACCAA | | | | | | | | | | | | | 10-123 |
| W908X | VRER- | CGUUCAUGUGCCA | 1605 | (AGCG) | 20 (C12) | 4.8 | 50 | 52 | 83 | 6 | 55 | 56 | 4 | + | 1-0-0-0- |
| | SpBE3 | CCGUGGG | | | | | | | | | | | | | 1 |
| W1161X | St1BE3 | AACCUCCAUACAC | 1606 | (CAAGAAA) | 20 (C7) | 3.4 | 49 | 61 | 84 | 25 | 77 | 29 | 3 | - | 1-0-0-0- |
| | | AACCUGA | | | | | | | | | | | | | 10 |
| W1245X | St1BE3 | UAUCCAUUUUAGA | 1607 | (CCAGAAT) | 20 (C5) | 4.5 | 49 | 61 | 84 | 25 | 77 | 29 | 3 | - | 1-0-0-0- |
| | | AGCAUUU | | | | | | | | | | | | | 10 |
| W1408X | St1BE3 | CAUCCCUUAAAAG | 1608 | (TAAGAAT) | 20 (C1) | 7.2 | 96 | 34 | 37 | 6 | 42 | 29 | 7 | - | 0-0-0-1- |
| | | UUGCCUU | | | | | | | | | | | | | 28 |
| Q1494X | SpBE3 | ACAAAAGCCAAUU | 1609 | (CAG) | 20 (C2) | 5.1 | 85 | 48 | 8 | 14 | 50 | 38 | 5 | + | 0-0-0- |
| | | CCUCGAC | | | | | | | | | | | | | 10-75 |
| Q1494X | SpBE3 | CAAAAGCCAAUUC | 1610 | (AGG) | 20 (C1) | 3.5 | 81 | 52 | 25 | 6 | 71 | 37 | 3 | + | 0-0-3- |
| | | CUCGACC | | | | | | | | | | | | | 12-94 |
| Q25X | SpBE3 | AUUGAACAACGCA | 1611 | (AAG) | 20 (C7) | 8.3 | 61 | 58 | 70 | 11 | 65 | 26 | 8 | - | 01-1- |
| | | UUGCUGA | | | | | | | | | | | | | 15-82 |
| Q1462X | St1BE3 | GAUAAUUUCAACC | 1612 | (AAAGAAG) | 20 (C9) | 3.4 | 43 | 47 | 88 | 35 | 76 | 14 | 3 | - | 0-2-2-9- |
| | | AACAGAA | | | | | | | | | | | | | 143 |
| Q240X | KKH- | UCCAGUCAGUGAA | 1613 | (TCTGAT) | 20 (C3) | 4.9 | 84 | 36 | 45 | 7 | 48 | 36 | 4 | - | 0-0-0-4- |
| | SaBE3 | GAAGCUU | | | | | | | | | | | | | 15 |
| Q408/ | St1BE3 | AACAGAACCAGGC | 1614 | (GAAGAAG) | 20 (C3/9) | 4.1 | 86 | 43 | 28 | 7 | 50 | 41 | 4 | - | 0-0-1-5- |
| 10X | | AAACAUU | | | | | | | | | | | | | 65 |
| Q643X | SpBE3 | CAAUGGACAGCUU | 1615 | (AGG) | 20 (C8) | 4.2 | 68 | 61 | 61 | 15 | 67 | 54 | 4 | + | 0-0-0- |
| | | CUGCCAG | | | | | | | | | | | | | 21-133 |
| Q708X | SpBE3 | CCAGACAAAAAUG | 1616 | (TGG) | 20 (C6) | 7.2 | 62 | 67 | 55 | 13 | 73 | 47 | 7 | + | 0-0-5- |
| | | UCCACCU | | | | | | | | | | | | | 24-165 |
| Q708X | KKH- | CAGACAAAAAUGU | 1617 | (GGTGGT) | 20 (C5) | 4.2 | 84 | 45 | 32 | 0 | 46 | 70 | 4 | - | 0-0-0-4- |
| | SaBE3 | CCACCUU | | | | | | | | | | | | | 20 |
| R841X | SpBE3 | UUAGCUCCGAGUC | 1618 | (TGG) | 20 (C8) | 6.4 | 67 | 62 | 34 | 59 | 64 | 47 | 6 | - | 0-1-0-6- |
| | | UUCAAGU | | | | | | | | | | | | | 44 |
| Q1862X | SpBE3 | UUCGUUCACAGAU | 1619 | (AGG) | 20 (C9) | 3.9 | 49 | 50 | 80 | 5 | 42 | 43 | 3 | - | 0-0-4- |
| | | GGAAGAA | | | | | | | | | | | | | 24-228 |
| W151X | SpBE3 | CCAGUCCGGUGGG | 1620 | (TGG) | 20 (C2) | 7.9 | 87 | 41 | 41 | 2 | 43 | 43 | 7 | - | 0-0-1-4- |
| | | UUAUUCA | | | | | | | | | | | | | 35 |
| R523X | EQR- | GCAUAGGCGAGCA | 1621 | (AGAG) | 20 (C8) | 4.3 | 74 | 48 | 53 | 23 | 83 | 41 | 4 | - | 0-0-0-8- |
| | SpBE3 | CAUGAAA | | | | | | | | | | | | | 92 |
| Q1470X | St1BE3 | GAGGUCAAGACAU | 1622 | (ACAGAAG) | 20 (C6) | 4.8 | 46 | 54 | 81 | 7 | 65 | 44 | 4 | - | 1-1-0-2- |
| | | CUUUAUG | | | | | | | | | | | | | 25 |
| W1578X | VQR- | CCAUCCUACAGUG | 1623 | (AGTG) | 20 (C2) | 5.2 | 78 | 48 | 35 | 25 | 37 | 44 | 5 | - | 0-0-0- |
| | SpBE3 | AAGUAGU | | | | | | | | | | | | | 17-112 |
| Q1026X | SaBE3 | AGAUAAGACAAGC | 1624 | (CTGAAT) | 20 (C9) | 4.9 | 69 | 56 | 51 | 31 | 66 | 40 | 4 | - | 0-0-1-7- |
| | | AGAAGAU | | | | | | | | | | | | | 40 |
| Q1077X | KKH- | GUGAUGGUCAAUC | 1625 | (CACAAT) | 20 (C9) | 5.2 | 90 | 35 | 9 | 26 | 28 | 44 | 5 | - | 0-0-0-2- |
| | SaBE3 | AUUUAUU | | | | | | | | | | | | | 21 |
| Q58X | SpBE3 | AACAGCUGCCCUU | 1626 | (GGG) | 20 (C3) | 6.2 | 74 | 49 | 3 | 7 | 24 | 25 | 6 | - | 0-0-1- |
| | | CAUCUAU | | | | | | | | | | | | | 10-115 |
| W151X | KKH- | GUCCAGUCCGGUG | 1627 | (CATGGT) | 20 (C4) | 4.9 | 100 | 23 | 7 | 4 | 9 | 53 | 4 | - | 0-0-0-1- |
| | SaBE3 | GGUUAUU | | | | | | | | | | | | | 0 |
| Q323X | SpBE3 | UGUGUAGUCAGUG | 1628 | (GGG) | 20 (C9) | 7.9 | 43 | 75 | 80 | 69 | 68 | 76 | 7 | + | 0-1-2-62 |
| | | UCCAGAG | | | | | | | | | | | | | 35-1 |
| Q485X | EQR- | AUCAAAAGAAGCU | 1629 | (GGAG) | 20 (C3) | 3.6 | 62 | 60 | 53 | 35 | 44 | 30 | 3 | - | 0-0-3- |
| | SpBE3 | CUCCAGU | | | | | | | | | | | | | 14-187 |
| Q1167X | SpBE3 | UGCCAAGUUAACA | 1630 | (AGG) | 20 (C4) | 6.0 | 76 | 46 | 15 | 1 | 29 | 41 | 6 | - | 0-0-2-8- |
| | | UAGAGUC | | | | | | | | | | | | | 89 |
| Q1515X | VQR- | CUAGUGACAAAUC | 1631 | (TGAT) | 20 (C8) | 4.8 | 74 | 48 | 12 | 13 | 63 | 52 | 4 | - | 0-0-0- |
| | SpBE3 | AAGCCUU | | | | | | | | | | | | | 17-129 |
| Q1167X | EQR- | UCAUGCUGCCAAG | 1632 | (AGAG) | 20 (C10) | 8.6 | 64 | 53 | 57 | 43 | 69 | 27 | 8 | - | 0-0-1- |
| | SpBE3 | UUAACAU | | | | | | | | | | | | | 27-191 |
| Q805X | SpBE3 | AUGAGUAUUUCCA | 1633 | (TGG) | 20 (C12) | 4.4 | 67 | 51 | 39 | 7 | 51 | 32 | 4 | + | 0-0-2- |
| | | AGUAGGC | | | | | | | | | | | | | 25-177 |
| Q360X | SpBE3 | GGCUAAUGACCCA | 1634 | (TGG) | 20 (C12) | 6.1 | 83 | 28 | 33 | 5 | 28 | 10 | 6 | - | 0-0-1-7- |
| | | AGAUUAC | | | | | | | | | | | | | 57 |
| Q323X | EQR- | CUCGUGUGUAGUC | 1635 | (AGAG) | 20 (C13) | 4.4 | 75 | 40 | 5 | 1 | 34 | 46 | 4 | + | 0-0-1- |
| | SpBE3 | AGUGUCC | | | | | | | | | | | | | 10-63 |
| W730X | KKH- | AUCCAAUAUGGAG | 1636 | (CCAGAT) | 20 (C4) | 4.1 | 64 | 39 | 51 | 5 | 19 | 44 | 4 | - | 0-0-2- |
| | SaBE3 | AGCAAUU | | | | | | | | | | | | | 11-36 |
| R214X | EQR- | UUCGAACUUUCAG | 1637 | (AGAG) | 20 (C3) | 4.7 | 42 | 49 | 72 | 2 | 33 | 28 | 4 | - | 0-2-2- |
| | SpBE3 | AGUAUUG | | | | | | | | | | | | | 14-188 |
| Q265X | VQR- | ACAGCUGUUCAUG | 1638 | (TGAA) | 20 (C2) | 8.1 | 61 | 53 | 43 | 10 | 50 | 33 | 8 | - | 0-0-4- |
| | SpBE3 | GGAAACC | | | | | | | | | | | | | 15-185 |
| Q687X | EQR- | UCAGACAGAGAGC | 1639 | (AGAG) | 20 (C5) | 4.0 | 55 | 56 | 51 | 34 | 77 | 26 | 4 | - | 0-0-3- |
| | SpBE3 | AAUGAGU | | | | | | | | | | | | | 30-285 |
| W908X | St3BE3 | AGUCGUUCAUGUG | 1640 | (GGGAG) | 20 (C15) | 5.7 | 48 | 63 | 24 | 22 | 54 | 63 | 5 | + | 1-0-1-3- |
| | | CCACCGU | | | | | | | | | | | | | 7 |
| W363X | SpBE3 | AAGGUUUUCCCAG | 1641 | (GGG) | 20 (C11) | 8.2 | 64 | 46 | 28 | 10 | 55 | 46 | 8 | - | 0-0-5- |
| | | UAAUCUU | | | | | | | | | | | | | 17-172 |
| Q1401X | SpBE3 | UAAAGGCAACUUU | 1642 | (TGG) | 20 (C7) | 7.3 | 53 | 49 | 49 | 14 | 83 | 37 | 7 | - | 0-0-3- |
| | | UAAGGGA | | | | | | | | | | | | | 37-245 |
| W908X | SpBE3 | UGUGCCACCGUGG | 1643 | (CAG) | 20 (C6) | 5.5 | 48 | 52 | 41 | 33 | 34 | 77 | 5 | - | 1-0-0-2- |
| | | GAGCGUA | | | | | | | | | | | | | 49 |
| W730X | EQR- | UUGAAUUUUAUCC | 1644 | (AGAG) | 20 (C13) | 3.0 | 41 | 52 | 54 | 6 | 68 | 29 | 3 | - | 0-1-3- |
| | SpBE3 | AAUAUGG | | | | | | | | | | | | | 33-405 |
| W808X | SpBE3 | CAAAAAUAUUCCA | 1645 | (TGG) | 20 (C12) | 3.8 | 61 | 34 | 31 | 1 | 22 | 34 | 3 | - | 0-1-4- |
| | | GCCUACU | | | | | | | | | | | | | 18-174 |
| Q1026X | VQR- | GAUAAGACAAGCA | 1646 | (TGAA) | 20 (C8) | 3.9 | 49 | 41 | 46 | 5 | 76 | 24 | 3 | - | 0-0-1- |
| | SpBE3 | GAAGAUC | | | | | | | | | | | | | 32-348 |
| R214X | EQR- | UCUUCGAACUUUC | 1647 | (TGAG) | 20 (C5) | 5.4 | 56 | 38 | 19 | 11 | 33 | 30 | 5 | - | 0-1-3- |
| | SpBE3 | AGAGUAU | | | | | | | | | | | | | 10-139 |

^{a}BE types: SpBE3 = APOBEC1-SpCas9n-UGI; VQR-SpBE3 = APOBEC1-VQR-SpCas9n-UGI; EQR-SpBE3 = APOBEC1-EQR-SpCas9n-UGI; VRER-SpBE3 = APOBEC1-VRER-SpCas9n-UGI; SaBE3 = APOBEC1-SaCas9n-UGI; KKH-SaBE3 = APOBEC1-KKH-SaCas9n-UGI; St3BE3 = APOBEC1-St3Cas9n-UGI; St1BE3 = APOBEC1-St1Cas9n-UGI.
^{b}Efficiency score, based on Housden et al (Science Signaling, 2015, 8 (393):rs9).
^{c}Specificity scores based on Hsu et al (Nature biotechnology, 2013, 31 (9):827-832), Fusi et al (bioRxiv 021568; doi: http://dx.doi.org/10.1101/021568), Chari et al (Nature Methods, 2015, 12 (9):823-6), Doench et al (Nature Biotechnology, 2014, 32 (12):1262-7), Wang et al (Science, 2014, 343 (6166): 80-4), Moreno-Mateos et al (Nature Methods, 2015, 12 (10)982-8), Housden et al (Science Signaling, 2015, 8 (393):rs9), and the “Prox/GC” column shows “+” if the proximal 6 bp to the PAM has a GC count > = 4, and GG if the guide ends with GG, based on Farboud et al (Genetics, 2015, 199 (4):959-71).
^{d}Number of predicted off-target binding sites in the human genome allowing up to 0, 1, 2, 3 or 4 mismatches, respectively shown in the format 0-1-2-3-4. Algorithm used: Haeussler et al, Genome Biol. 2016; 17: 148.

TABLE 8
Exemplary Efficiency and Specificity Scores for gRNAs for NaV1.7 (SCN9A) Protective Loss-of-Function Mutations via Codon
Change

| | | Programmable | SEQ | | | | | | | | | | | Pro | |
|---|---|---|---|---|---|---|---|---|---|---|---|---|---|---|---|
| Target | BE | guide-RNA | ID | | gRNA size | | | | | | | M.- | Hous- | x/ | Off |
| variants | type^{a} | sequence | NOs | PAM | (C edited) | Eff^{b} | Hsu^{c} | Fusi | Chari | Doench | Wang | M. | den | GC | targets^{d} |
| C324Y | St1BE3 | GGACACUGACUACA | 1648 | (AAAGAAC) | 20 (C7/8) | 6.2 | 99 | 68 | 99 | 61 | 84 | 36 | 6 | + | 0-0-0- |
| | | CACGAG | | | | | | | | | | | | | 0-9 |
| P613L/S/F | KKH- | CCAAUGCUGCCGGU | 1649 | (GAAAAT) | 20 (C5) | 9.2 | 100 | 63 | 97 | 70 | 88 | 71 | 9 | + | 0-0-0- |
| | SaBE3 | GAACGG | | | | | | | | | | | | | 0-2 |
| P591L/S/F | VRER- | UGUGCCCCACAGAC | 1650 | (AGCG) | 20 (C8) | 7.8 | 97 | 64 | 96 | 18 | 84 | 69 | 7 | + | 0-0-0- |
| | SpBE3 | CCCAGG | | | | | | | | | | | | | 1-13 |
| G785R | St1BE3 | GAUUCCAGUAAAGA | 1651 | (TGAGAAA) | 20 (C9) | 6.3 | 94 | 59 | 99 | 24 | 78 | 42 | 6 | - | 0-0-0- |
| | | CCUAAG | | | | | | | | | | | | | 1-52 |
| P683L/S/F | KKH- | GAUCCCAACCUCAG | 1652 | (AGCAAT) | 20 (C14) | 6.2 | 92 | 66 | 99 | 39 | 77 | 26 | 6 | - | 0-0-0- |
| | SaBE3 | ACAGAG | | | | | | | | | | | | | 2-13 |
| C925Y | KKH- | UCUCCACACAGCAC | 1653 | (CACAAT) | 20 (C2) | 4.7 | 98 | 62 | 87 | 26 | 59 | 45 | 4 | + | 0-0-0- |
| | SaBE3 | GCGGAA | | | | | | | | | | | | | 0-11 |
| P1712/3L/ | KKH- | CCACCCGACUGUGA | 1654 | (AAAAGT) | 20 (C3/4/12) | 6.1 | 99 | 57 | 86 | 18 | 39 | 61 | 6 | - | 0-0-0- |
| S/F | SaBE3 | cCCAAA | | | | | | | | | | | | | 0-5 |
| P1606L/S/F | KKH- | GUGUCCCCUACCCU | 1655 | (AGTGAT) | 20 (C13114) | 6.4 | 93 | 56 | 92 | 50 | 76 | 41 | 6 | + | 0-0-0- |
| | SaBE3 | GUUCCG | | | | | | | | | | | | | 1-13 |
| P983L/S/F | KKH- | GACCCUGAUGCAAA | 1656 | (CCAGAT) | 20 (C819) | 3.5 | 96 | 58 | 82 | 9 | 74 | 24 | 3 | - | 0-0-0- |
| | SaBE3 | CAACCU | | | | | | | | | | | | | 2-17 |
| P591L/S/F | St3BE3 | GUUUGUGCCCCACA | 1657 | (AGGAG) | 20 (C7-14) | 7.0 | 90 | 55 | 86 | 45 | 74 | 38 | 7 | + | 0-0-0- |
| | | GACCCC | | | | | | | | | | | | | 3-38 |
| P532L/S/F | KKH- | UACCCCCAAUCAGG | 1658 | (CCAAAT) | 20 (C6-13) | 4.7 | 96 | 56 | 77 | 5 | 47 | 48 | 4 | - | 0-0-0- |
| | SaBE3 | UACCAC | | | | | | | | | | | | | 0-3 |
| P1606L/S/F | VQR- | GUGUCCCCUACCCU | 1659 | (AGTG) | 20 (C5-12) | 6.4 | 81 | 56 | 92 | 50 | 76 | 41 | 6 | + | 0-0-1- |
| | SpBE3 | GUUCCG | | | | | | | | | | | | | 7-62 |
| P1496/8L/ | SpBE3 | AAAAGCCAAUUCCU | 1660 | (GGG) | 20 (C5-12) | 6.0 | 87 | 61 | 85 | 14 | 64 | 56 | 6 | + | 0-0-2- |
| S/F | | CGACCA | | | | | | | | | | | | | 12-86 |
| P1133/5L/ | St1BE3 | AUAACCCUUUGCCU | 1661 | (GGAGAAG) | 20 (C7-14) | 6.5 | 90 | 53 | 81 | 33 | 46 | 53 | 6 | - | 0-0-0- |
| S/F | | GGAGAA | | | | | | | | | | | | | 4-44 |
| P111L/S/F | SaBE3 | UCUCCUUUCAGUCC | 1662 | (AAGAAT) | 20 (C7-14) | 4.4 | 88 | 47 | 81 | 69 | 37 | 24 | 4 | - | 0-0-1- |
| | | UCUAAG | | | | | | | | | | | | | 4-27 |
| P229L/S/F | KKH- | GUAAUCCCAGGUAA | 1663 | (ATTGGT) | 20 (C5-12) | 4.3 | 88 | 45 | 81 | 26 | 64 | 45 | 4 | - | 0-0-1- |
| | SaBE3 | GAAGUA | | | | | | | | | | | | | 5-17 |
| P1791L/S/F | KKH- | GAAGUUUGAUCCCG | 1664 | (CCCAGT) | 20 (C11/12) | 2.6 | 98 | 59 | 67 | 37 | 92 | 59 | 2 | - | 0-0-0- |
| | SaBE3 | AUGCGA | | | | | | | | | | | | | 1-1 |
| C315Y | SaBE3 | AACCACAAAGGAGA | 1665 | (TTGGAT) | 20 (C8) | 4.6 | 89 | 47 | 76 | 1 | 40 | 14 | 4 | - | 0-0-0- |
| | | GCAUCU | | | | | | | | | | | | | 2-21 |
| C1154Y | SaBE3 | GAAACAGGCCUCUG | 1666 | (CGGAAT) | 20 (C15) | 6.6 | 90 | 55 | 74 | 17 | 46 | 66 | 6 | - | 0-0-0- |
| | | GCUCAU | | | | | | | | | | | | | 4-4 |
| P1133/5L/ | St1BE3 | ACCCUUUGCCUGGA | 1667 | (GAAGAAG) | 20 (C16) | 5.8 | 93 | 60 | 70 | 39 | 51 | 40 | 5 | - | 0-0-1- |
| S/F | | GAAGGA | | | | | | | | | | | | | 2-17 |
| G786R | EQR- | GAUUCCAGUAAAGA | 1668 | (TGAG) | 20 (C4) | 6.3 | 63 | 59 | 99 | 24 | 78 | 42 | 6 | - | 0-0-1- |
| | SpBE3 | CCUAAG | | | | | | | | | | | | | 28-179 |
| P1145L/S/F | SpBE3 | CUGAACCUAUGAAU | 1669 | (GAG) | 20 (C10) | 6.4 | 94 | 68 | 59 | 82 | 45 | 24 | 6 | - | 0-0-0- |
| | | UCCGAU | | | | | | | | | | | | | 1-117 |
| P609/10L/ | VQR- | GUCCCCACCAAUGC | 1670 | (TGAA) | 20 (C17) | 5.5 | 87 | 57 | 75 | 12 | 87 | 60 | 5 | + | 0-0-0- |
| S/F | SpBE3 | UGCCGG | | | | | | | | | | | | | 10-66 |
| P609/10L/ | VQR- | AGGUCCCCACCAAU | 1671 | (GGTG) | 20 (C11) | 8.7 | 84 | 49 | 78 | 14 | 43 | 13 | 8 | + | 0-0-0- |
| S/F | SpBE3 | GCUGCC | | | | | | | | | | | | | 9-85 |
| P1093L/S/F | KKH- | CACCUGGGGAAUCC | 1672 | (GAAAAT) | 20 (C12) | 6.5 | 98 | 59 | 62 | 2 | 36 | 60 | 6 | - | 0-0-0- |
| | SaBE3 | GAUUUG | | | | | | | | | | | | | 0-8 |
| C944Y | VRER- | AUAAGGCACAUAGC | 1673 | (AGCG) | 20 (C13) | 4.8 | 100 | 60 | 48 | 12 | 66 | 21 | 4 | - | 0-0-0- |
| | SpBE3 | UUGACC | | | | | | | | | | | | | 1-6 |
| P337L/S/F | EQR- | AAACCCUGAUUAUG | 1674 | (CGAG) | 20 (C13) | 3.9 | 77 | 63 | 82 | 7 | 29 | 28 | 3 | - | 0-0-0- |
| | SpBE3 | GCUACA | | | | | | | | | | | | | 13-121 |
| P594L/S/F | SpBE3 | CACAGACCCCAGGA | 1675 | (CAG) | 20 (C5) | 7.7 | 82 | 59 | 77 | 65 | 72 | 64 | 7 | + | 0-0-2- |
| | | GCGACG | | | | | | | | | | | | | 17-142 |
| P80L/S/F | KKH- | GACCCCUACUAUGC | 1676 | (AAAGGT) | 20 (C6) | 4.5 | 95 | 62 | 55 | 12 | 70 | 48 | 4 | - | 0-0-0- |
| | SaBE3 | AGACAA | | | | | | | | | | | | | 2-7 |
| P80L/S/F | St3BE3 | CCCCUACUAUGCAG | 1677 | (AGGTG) | 20 (C7) | 3.5 | 92 | 37 | 65 | 27 | 24 | 42 | 3 | - | 0-0-0- |
| | | ACAAAA | | | | | | | | | | | | | 4-31 |
| P60L/S/F | St3BE3 | AAACAGCUGCCCUU | 1678 | (TGGGG) | 20 (C11) | 8.4 | 96 | 26 | 61 | 3 | 53 | 35 | 8 | - | 0-0-0- |
| | | CAUCUA | | | | | | | | | | | | | 3-29 |
| P1490S^{d} | KKH- | GGGUCCAAGAAGCC | 1679 | (GCCAAT) | 20 (C13) | 5.0 | 75 | 57 | 81 | 64 | 74 | 57 | 5 | - | 0-0-0- |
| | SaBE3 | ACAAAA | | | | | | | | | | | | | 2-28 |
| P594L/S/F | KKH- | ACAGACCCCAGGAG | 1680 | (AGCAGT) | 20 (C14) | 4.3 | 97 | 52 | 58 | 14 | 77 | 55 | 4 | + | 0-0-0- |
| | SaBE3 | CGACGC | | | | | | | | | | | | | 2-12 |
| C324Y | VQR- | CUGGACACUGACUA | 1681 | (AGAA) | 20 (C7) | 5.5 | 85 | 69 | 69 | 75 | 67 | 61 | 5 | + | 0-0-1- |
| | SpBE3 | CACACG | | | | | | | | | | | | | 7-86 |
| G830R | St1BE3 | UGACAAUCCUUCCA | 1682 | (CTAGAAA) | 20 (C16) | 4.1 | 96 | 58 | 58 | 15 | 54 | 36 | 4 | - | 0-0-0- |
| | | CAUCUG | | | | | | | | | | | | | 2-13 |
| C1526Y | KKH- | AGACAGAUAAGAAC | 1683 | (ACTAAT) | 20 (C5) | 4.5 | 87 | 54 | 67 | 41 | 53 | 20 | 4 | - | 0-0-0- |
| | SaBE3 | CAUGAU | | | | | | | | | | | | | 3-26 |
| P850L/S/F | KKH- | UCCUGGCCAACAUU | 1684 | (GCTGAT) | 20 (C6) | 5.4 | 94 | 58 | 52 | 31 | 55 | 43 | 5 | - | 0-0-0- |
| | SaBE3 | GAACAU | | | | | | | | | | | | | 2-15 |
| P67L/S/F | St3BE3 | UGGGGACAUUCCUC | 1685 | (TGGTG) | 20 (C9) | 4.4 | 96 | 56 | 34 | 5 | 61 | 77 | 4 | + | 0-0-0- |
| | | CCGGCA | | | | | | | | | | | | | 0-29 |
| P148L/S/F | KKH- | AUAACCCACCGGAC | 1686 | (AAAAAT) | 20 (C12) | 6.5 | 99 | 52 | 46 | 17 | 52 | 28 | 6 | + | 0-0-0- |
| | SaBE3 | UGGACC | | | | | | | | | | | | | 0-4 |
| P1133/5L/ | St1BE3 | CAGUUGAUAACCCU | 1687 | (GGAGAAG) | 20 (C14) | 5.3 | 97 | 54 | 16 | 22 | 75 | 31 | 5 | - | 0-0-0- |
| S/F | | UUGCCU | | | | | | | | | | | | | 0-25 |
| P325L/S/F | VQR- | AGUGUCCAGAGGGG | 1688 | (TGTG) | 20 (C8) | 6.9 | 90 | 54 | 60 | 3 | 54 | 47 | 6 | - | 0-0-0- |
| | SpBE3 | UACACC | | | | | | | | | | | | | 5-64 |
| P148L/S/F | SpBE3 | CCAUGAAUAACCCA | 1689 | (TGG) | 20 (C11) | 4.4 | 98 | 52 | 46 | 18 | 56 | 43 | 4 | + | 0-0-0- |
| | | CCGGAC | | | | | | | | | | | | | 3-24 |
| P1090L/S/F | St3BE3 | GUGACAGUGCCAAU | 1690 | (TGGGG) | 20 (C14) | 9.0 | 98 | 50 | 51 | 16 | 59 | 10 | 9 | + | 0-0-0- |
| | | UGCACC | | | | | | | | | | | | | 1-20 |
| P1498/1500 | KKH- | AAUUCCUCGACCAG | 1691 | (AAAAAT) | 20 (C9110) | 5.6 | 99 | 50 | 33 | 10 | 43 | 54 | 5 | - | 0-0-0- |
| L/S/F | SaBE3 | GGGUAA | | | | | | | | | | | | | 0-2 |
| S1490F^{d} | SpBE3 | UGGGGUCCAAGAAG | 1692 | (AAG) | 20 (C819) | 4.2 | 63 | 64 | 85 | 16 | 74 | 64 | 4 | - | 0-0-3- |
| | | CCACAA | | | | | | | | | | | | | 18-214 |
| P1090L/S/F | SpBE3 | UGACAGUGCCAAUU | 1693 | (GGG) | 20 (C7/8) | 7.4 | 80 | 63 | 68 | 42 | 71 | 30 | 7 | + | 0-0-1- |
| | | GCACCU | | | | | | | | | | | | | 12-120 |
| P1018L/S/F | St3BE3 | CAAAAAGCCAAAGA | 1694 | (GGGAG) | 20 (C5/6) | 4.5 | 87 | 55 | 61 | 40 | 77 | 30 | 4 | - | 0-0-0- |
| | | UUUCCA | | | | | | | | | | | | | 7-74 |
| G1626R | SpBE3 | UUGCUCCUUUGACU | 1695 | (AGG) | 20 (C10/11) | 5.6 | 86 | 62 | 42 | 43 | 59 | 56 | 5 | - | 0-0-4- |
| | | AGACGU | | | | | | | | | | | | | 5-68 |
| P711/2L/ | KKH- | AAAAUGUCCACCUU | 1696 | (ACAGAT) | 20 (C7/8) | 5.6 | 91 | 57 | 29 | 6 | 47 | 29 | 5 | + | 0-0-1- |
| S/F | SaBE3 | GGUGGU | | | | | | | | | | | | | 2-28 |
| C140Y | KKH- | AUAAAUAUGCAGUU | 1697 | (AATAGT) | 20 (C-1) | 9.0 | 77 | 60 | 70 | 35 | 60 | 54 | 9 | - | 0-0-0- |
| | SaBE3 | UGUCAG | | | | | | | | | | | | | 5-34 |
| P1083L/S/F | KKH- | CACAAUCCCAGCCU | 1698 | (GACAGT) | 20 (C5) | 6.0 | 85 | 62 | 50 | 18 | 62 | 45 | 6 | - | 0-0-0- |
| | SaBE3 | CACAGU | | | | | | | | | | | | | 2-28 |
| G1626R | SaBE3 | UUUGCUCCUUUGAC | 1699 | (TAGGAT) | 20 (C6) | 4.4 | 91 | 56 | 43 | 49 | 70 | 29 | 4 | - | 0-0-3- |
| | | UAGACG | | | | | | | | | | | | | 3-6 |
| P609/10L/ | KKH- | AGUAGGUCCCCACC | 1700 | (GCCGGT) | 20 (C7) | 6.1 | 97 | 50 | 39 | 21 | 61 | 48 | 6 | - | 0-0-0- |
| S/F | SaBE3 | AAUGCU | | | | | | | | | | | | | 1-7 |
| P1496/8L/ | SaBE3 | CAAAAGCCAAUUCC | 1701 | (AGGGGT) | 20 (C12) | 3.5 | 95 | 52 | 25 | 6 | 71 | 37 | 3 | + | 0-0-0- |
| S/F | | UCGACC | | | | | | | | | | | | | 1-7 |
| P60L/S/F | SpBE3 | ACAGCUGCCCUUCA | 1702 | (GGG) | 20 (C13) | 6.7 | 69 | 71 | 77 | 39 | 52 | 44 | 6 | - | 0-0-4- |
| | | UCUAUG | | | | | | | | | | | | | 19-191 |
| P1133/5L/ | St3BE3 | ACAGUUGAUAACCC | 1703 | (TGGAG) | 20 (C7) | 4.7 | 94 | 52 | 29 | 12 | 62 | 11 | 4 | - | 0-0-1- |
| S/F | | UUUGCC | | | | | | | | | | | | | 3-20 |
| P609/10L/ | St3BE3 | UAGGUCCCCACCAA | 1704 | (CGGTG) | 20 (C3) | 4.6 | 94 | 52 | 15 | 12 | 61 | 46 | 4 | + | 0-0-0- |
| S/F | | UGCUGC | | | | | | | | | | | | | 5-24 |
| P1145L/S/F | EQR- | GCUGAACCUAUGAA | 1705 | (TGAG) | 20 (C4) | 3.4 | 87 | 58 | 55 | 32 | 90 | 29 | 3 | - | 0-0-1- |
| | SpBE3 | UUCCGA | | | | | | | | | | | | | 2-79 |
| P1151L/S/F | KKH- | AGCCAGAGGCCUGU | 1706 | (GATGGT) | 20 (C8) | 9.0 | 90 | 55 | 46 | 8 | 43 | 31 | 9 | - | 0-0-0- |
| | SaBE3 | UUCACA | | | | | | | | | | | | | 1-20 |
| P1090L/S/F | SpBE3 | GACAGUGCCAAUUG | 1707 | (GGG) | 20 (C4/5) | 5.3 | 70 | 57 | 74 | 5 | 74 | 62 | 5 | + | 0-0-1- |
| | | CACCUG | | | | | | | | | | | | | 20-184 |
| P1133/5L/ | St3BE3 | GAUAACCCUUUGCC | 1708 | (AGGAG) | 20 (C5) | 3.7 | 93 | 51 | 42 | 12 | 82 | 33 | 3 | - | 0-0-0- |
| S/F | | UGGAGA | | | | | | | | | | | | | 3-35 |
| P1955/6L/ | KKH- | CCACCUCUCCACCU | 1709 | (GATAGT) | 20 (C8) | 8.1 | 92 | 52 | 42 | 63 | 37 | 38 | 8 | - | 0-0-0- |
| S/F | SaBE3 | UCAUAU | | | | | | | | | | | | | 1-12 |
| P1496L/S/F | St3BE3 | CAAAAGCCAAUUCC | 1710 | (AGGGG) | 20 (C9) | 3.5 | 92 | 52 | 25 | 6 | 71 | 37 | 3 | + | 0-0-2- |
| | | UCGACC | | | | | | | | | | | | | 1-22 |
| P1360L/S/F | KKH- | GGUUUCCUGCAAGU | 1711 | (CCAAAT) | 20 (C13) | 10.8 | 90 | 47 | 53 | 24 | 57 | 48 | 10 | - | 0-0-0- |
| | SaBE3 | CAAGUU | | | | | | | | | | | | | 3-9 |
| C1154Y | SpBE3 | GAAACAGGCCUCUG | 1712 | (CGG) | 20 (C10) | 6.6 | 68 | 55 | 74 | 17 | 46 | 66 | 6 | - | 0-0-1- |
| | | GCUCAU | | | | | | | | | | | | | 22-132 |
| P1722L/S/F | St3BE3 | CAUCCUGGAAGUUC | 1713 | (AGGAG) | 20 (C14) | 4.4 | 89 | 53 | 42 | 5 | 39 | 35 | 4 | - | 0-0-1- |
| | | AGUUGA | | | | | | | | | | | | | 5-40 |
| C1370Y | SpBE3 | GCAAAACAUUCGGA | 1714 | (TGG) | 20 (C2) | 3.9 | 95 | 37 | 47 | 14 | 64 | 26 | 3 | - | 0-0-1- |
| | | ACGAUU | | | | | | | | | | | | | 2-35 |
| P1773L/S/F | KKH- | CCUCUGAGUGAGGA | 1715 | (TGAGAT) | 20 (C3) | 5.0 | 92 | 50 | 25 | 16 | 40 | 42 | 5 | - | 0-0-1- |
| | SaBE3 | UGACUU | | | | | | | | | | | | | 3-12 |
| P60L/S/F | VQR- | CAGCUGCCCUUCAU | 1716 | (GGAC) | 20 (C4) | 6.0 | 76 | 65 | 47 | 21 | 68 | 67 | 6 | - | 0-0-0- |
| | SpBE3 | CUAUGG | | | | | | | | | | | | | 13-167 |
| G1736R | KKH- | UAUUCCAACAGAUG | 1717 | (CACAGT) | 20 (C10/11) | 5.5 | 95 | 42 | 46 | 1 | 19 | 39 | 5 | - | 0-0-0- |
| | SaBE3 | GGUUAC | | | | | | | | | | | | | 1-10 |
| P1093L/S/F | VQR- | GCACCUGGGGAAUC | 1718 | (GGAA) | 20 (C6/7) | 5.4 | 93 | 36 | 48 | 6 | 36 | 59 | 5 | - | 0-0-1- |
| | SpBE3 | CGAUUU | | | | | | | | | | | | | 2-54 |
| P1133/5L/ | EQR- | AUAACCCUUUGCCU | 1719 | (GGAG) | 20 (C12/13) | 6.5 | 59 | 53 | 81 | 33 | 46 | 53 | 6 | - | 0-0-2- |
| S/F | SpBE3 | GGAGAA | | | | | | | | | | | | | 22-182 |
| P187L/S/F | SaBE3 | UUCGUGACCCGUGG | 1720 | (CTGGAT) | 20 (C12-14) | 7.2 | 87 | 53 | 51 | 10 | 73 | 58 | 7 | - | 0-0-2- |
| | | AACUGG | | | | | | | | | | | | | 2-5 |
| C1690Y | KKH- | AGGCAAAUCAUACU | 1721 | (AAAGGT) | 20 (C10/11) | 6.3 | 91 | 49 | 43 | 19 | 36 | 33 | 6 | + | 0-0-1- |
| | SaBE3 | GUUGCC | | | | | | | | | | | | | 1-19 |
| P229L/S/F | St3BE3 | AAUCCCAGGUAAGA | 1722 | (TGGTG) | 20 (C1-5) | 7.1 | 83 | 38 | 57 | 9 | 38 | 54 | 7 | - | 0-0-1- |
| | | AGUAAU | | | | | | | | | | | | | 4-47 |
| C330Y | VQR- | UCACACAGGUGUAC | 1723 | (GGAC) | 20 (C213) | 5.8 | 85 | 55 | 38 | 2 | 27 | 51 | 5 | + | 0-0-0- |
| | SpBE3 | cccucu | | | | | | | | | | | | | 9-101 |
| G1577R | KKH- | AUUCCAUCCUACAG | 1724 | (AGTAGT) | 20 (C-1) | 4.4 | 89 | 51 | 15 | 15 | 37 | 35 | 4 | - | 0-0-0- |
| | SaBE3 | UGAAGU | | | | | | | | | | | | | 1-20 |
| C324Y | St1BE3 | CUCUGGACACUGAC | 1725 | (CGAGAAA) | 20 (C1) | 5.8 | 87 | 52 | 23 | 34 | 45 | 21 | 5 | - | 0-0-1- |
| | | UACACA | | | | | | | | | | | | | 3-46 |
| G1626R | VQR- | UGCUCCUUUGACUA | 1726 | (GGAT) | 20 (C13) | 4.8 | 89 | 49 | 48 | 50 | 26 | 51 | 4 | - | 0-0-3- |
| | SpBE3 | GACGUA | | | | | | | | | | | | | 5-66 |
| C275Y | KKH- | AUUUCGAAAACAUU | 1727 | (TCAGGT) | 20 (C15) | 5.8 | 83 | 45 | 55 | 7 | 52 | 14 | 5 | - | 0-0-0- |
| | SaBE3 | UAUGCU | | | | | | | | | | | | | 3-48 |
| P1093L/S/F | SpBE3 | UGCACCUGGGGAAU | 1728 | (TGG) | 20 (C16) | 7.0 | 94 | 34 | 44 | 0 | 29 | 37 | 7 | - | 0-0-0- |
| | | CCGAUU | | | | | | | | | | | | | 4-48 |
| P683L/S/F | EQR- | AUGAUCCCAACCUC | 1729 | (AGAG) | 20 (C1) | 3.8 | 66 | 61 | 71 | 36 | 80 | 57 | 3 | - | 0-0-1- |
| | SpBE3 | AGACAG | | | | | | | | | | | | | 18-162 |
| P1018L/S/F | KKH- | AAAAAGCCAAAGAU | 1730 | (GGAGAT) | 20 (C18) | 5.8 | 49 | 57 | 88 | 27 | 91 | 43 | 5 | - | 0-0-1- |
| | SaBE3 | UUCCAG | | | | | | | | | | | | | 12-27 |
| P1090L/S/F | SpBE3 | GUGACAGUGCCAAU | 1731 | (TGG) | 20 (C2) | 9.0 | 86 | 50 | 51 | 16 | 59 | 10 | 9 | + | 0-0-2- |
| | | UGCACC | | | | | | | | | | | | | 8-82 |
| P609/10L/ | SpBE3 | CCACCAAUGCUGCC | 1732 | (CGG) | 20 (C16) | 7.3 | 87 | 50 | 49 | 19 | 22 | 47 | 7 | - | 0-0-1- |
| S/F | | GGUGAA | | | | | | | | | | | | | 7-85 |
| P1319L/S/F | VQR- | GCAAUUCCUUCCAU | 1733 | (TGTG) | 20 (C415) | 5.9 | 63 | 55 | 73 | 16 | 64 | 28 | 5 | - | 0-0-2- |
| | SpBE3 | CAUGAA | | | | | | | | | | | | | 18-223 |
| P536L/S/F | SpBE3 | CAGUCACCACUCAG | 1734 | (TGG) | 20 (C3/4) | 7.0 | 81 | 43 | 55 | 9 | 45 | 39 | 7 | - | 0-0-1- |
| | | CAUUCG | | | | | | | | | | | | | 12-123 |
| P1297L/S/F | KKH- | AAGACCUCUAAGAG | 1735 | (CTAGAT) | 20 (C112) | 5.1 | 98 | 38 | 8 | 6 | 40 | 54 | 5 | - | 0-0-0- |
| | SaBE3 | CCUUAU | | | | | | | | | | | | | 1-5 |
| P60L/S/F | SpBE3 | AAACAGCUGCCCUU | 1736 | (TGG) | 20 (C314) | 8.4 | 74 | 26 | 61 | 3 | 53 | 35 | 8 | - | 0-0-2- |
| | | CAUCUA | | | | | | | | | | | | | 12-147 |
| P35L/S/F | St1BE3 | UCAAAGGAACCCAA | 1737 | (AAAGAAA) | 20 (C11/12) | 5.0 | 43 | 46 | 91 | 11 | 72 | 29 | 5 | - | 0-1-0- |
| | | AGAAGA | | | | | | | | | | | | | 21-224 |
| P67/8L/S/F | SpBE3 | UGGGGACAUUCCUC | 1738 | (TGG) | 20 (C7/8) | 4.4 | 78 | 56 | 34 | 5 | 61 | 77 | 4 | + | 0-0-0- |
| | | CCGGCA | | | | | | | | | | | | | 8-149 |
| P646L/S/F | KKH- | AGCUUCUGCCAGAG | 1739 | (ATAGAT) | 20 (C5/6) | 6.7 | 94 | 40 | 24 | 1 | 30 | 23 | 6 | - | 0-0-0- |
| | SaBE3 | GUGAUA | | | | | | | | | | | | | 3-16 |
| P1829L/S/F | St3BE3 | AUGGAUCUGCCCAU | 1740 | (TGGTG) | 20 (C4/5) | 10.0 | 68 | 49 | 65 | 10 | 39 | 59 | 10 | - | 0-2-3- |
| | | GGUUAG | | | | | | | | | | | | | 2-39 |
| C330Y | SpBE3 | UUCACACAGGUGUA | 1741 | (TGG) | 20 (C3/4) | 5.0 | 87 | 42 | 46 | 4 | 29 | 50 | 5 | + | 0-0-0- |
| | | CCCCUC | | | | | | | | | | | | | 8-88 |
| G1577R | SpBE3 | UCCAUCCUACAGUG | 1742 | (TAG) | 20 (C2/3) | 6.5 | 72 | 61 | 40 | 24 | 62 | 53 | 6 | - | 0-0-4- |
| | | AAGUAG | | | | | | | | | | | | | 11-122 |
| P1496/8L/ | SpBE3 | CAAAAGCCAAUUCC | 1743 | (AGG) | 20 (C1/2) | 3.5 | 81 | 52 | 25 | 6 | 71 | 37 | 3 | + | 0-0-3- |
| S/F | | UCGACC | | | | | | | | | | | | | 12-94 |
| C1328Y | KKH- | AGACACACAAGUAG | 1744 | (CATGAT) | 20 (C13/14) | 5.1 | 90 | 43 | 22 | 5 | 29 | 29 | 5 | - | 0-0-0- |
| | SaBE3 | CACAUU | | | | | | | | | | | | | 1-20 |
| P1496L/S/F | SpBE3 | ACAAAAGCCAAUUC | 1745 | (CAG) | 20 (C415) | 5.1 | 85 | 48 | 8 | 14 | 50 | 38 | 5 | + | 0-0-0- |
| | | CUCGAC | | | | | | | | | | | | | 10-75 |
| G1339R | St1BE3 | CCCAUGAUGCUGAA | 1746 | (CCAGAAT) | 20 (C7) | 6.9 | 62 | 64 | 70 | 41 | 63 | 39 | 6 | - | 0-2-3- |
| | | UAUCAG | | | | | | | | | | | | | 3-15 |
| P1717L/S/F | SpBE3 | GACCCAAAAAAAGU | 1747 | (TGG) | 20 (C13114) | 5.9 | 63 | 56 | 69 | 6 | 73 | 0 | 5 | - | 0-0-3- |
| | | UCAUCC | | | | | | | | | | | | | 18-120 |
| P591L/S/F | EQR- | UUUGUGCCCCACAG | 1748 | (GGAG) | 20 (C12/13) | 6.8 | 56 | 55 | 76 | 70 | 75 | 54 | 6 | + | 0-0-1- |
| | SpBE3 | ACCCCA | | | | | | | | | | | | | 32-223 |
| G1626R | SpBE3 | UUUGCUCCUUUGAC | 1749 | (TAG) | 20 (C13/14) | 4.4 | 76 | 56 | 43 | 44 | 70 | 29 | 4 | - | 0-0-2- |
| | | UAGACG | | | | | | | | | | | | | 24-72 |
| P114L/S/F | KKH- | CCUCUAAGAAGAAU | 1750 | (TAAGAT) | 20 (C3) | 6.1 | 93 | 38 | 30 | 16 | 37 | 0 | 6 | - | 0-0-0- |
| | SaBE3 | AUCUAU | | | | | | | | | | | | | 2-21 |
| P800L/S/F | KKH- | AUGGAUCCAUAUGA | 1751 | (CCAAGT) | 20 (C12) | 5.8 | 90 | 41 | 3 | 3 | 22 | 62 | 5 | - | 0-0-0- |
| | SaBE3 | GUAUUU | | | | | | | | | | | | | 2-18 |
| P1285L/S/F | SpBE3 | UUGGCCCCAUUAAA | 1752 | (CGG) | 20 (C13) | 4.5 | 77 | 52 | 46 | 51 | 45 | 39 | 4 | - | 0-0-1- |
| | | UCCCUU | | | | | | | | | | | | | 10-123 |
| G1626R | SpBE3 | CUUUGACUAGACGU | 1753 | (CGG) | 20 (C5) | 7.9 | 81 | 48 | 9 | 41 | 28 | 52 | 7 | - | 0-0-3- |
| | | AGGAUU | | | | | | | | | | | | | 2-53 |
| P111L/S/F | St1BE3 | UUCUCCUUUCAGUC | 1754 | (GAAGAAT) | 20 (C14) | 6.5 | 88 | 41 | 6 | 6 | 17 | 26 | 6 | - | 0-0-0- |
| | | CUCUAA | | | | | | | | | | | | | 8-47 |
| C944Y | SpBE3 | AAUAAGGCACAUAG | 1755 | (CAG) | 20 (C13/14) | 7.7 | 75 | 53 | 19 | 12 | 34 | 35 | 7 | - | 0-0-3- |
| | | CUUGAC | | | | | | | | | | | | | 7-76 |
| C753Y | KKH- | AACUAUGCAAAUGG | 1756 | (CAAGAT) | 20 (C12/13) | 5.0 | 79 | 48 | 36 | 2 | 54 | 34 | 5 | - | 0-0-2- |
| | SaBE3 | UAAUUG | | | | | | | | | | | | | 3-48 |
| P610L/S/F | SpBE3 | CACCAAUGCUGCCG | 1757 | (GGG) | 20 (C11/12) | 4.0 | 90 | 37 | 17 | 4 | 19 | 40 | 4 | - | 0-0-0- |
| | | GUGAAC | | | | | | | | | | | | | 6-66 |
| P1829L/S/F | SpBE3 | AUGGAUCUGCCCAU | 1758 | (TGG) | 20 (C10/11) | 10.0 | 60 | 49 | 65 | 10 | 39 | 59 | 10 | - | 0-2-4- |
| | | GGUUAG | | | | | | | | | | | | | 14-116 |
| P1090/3L/ | KKH- | UGCCAAUUGCACCU | 1759 | (TCCGAT) | 20 (C7/8) | 8.9 | 82 | 43 | 21 | 4 | 44 | 31 | 8 | + | 0-0-1- |
| S/F | SaBE3 | GGGGAA | | | | | | | | | | | | | 4-13 |
| P711/2L/ | VQR- | AAUGUCCACCUUGG | 1760 | (AGAT) | 20 (C112) | 8.2 | 82 | 43 | 7 | 5 | 36 | 56 | 8 | - | 0-0-0- |
| S/F | SpBE3 | UGGUAC | | | | | | | | | | | | | 12-94 |
| P5/6/7L/S/F | EQR- | GUUGCCUCCCCCAG | 1761 | (AGAG) | 20 (C1/2) | 7.0 | 57 | 34 | 67 | 2 | 53 | 31 | 7 | + | 0-0-1- |
| | SpBE3 | GACCUC | | | | | | | | | | | | | 23-184 |
| P1829L/S/F | KKH- | CCAUGGAUCUGCCC | 1762 | (AGTGGT) | 20 (C9/10) | 4.3 | 94 | 30 | 17 | 8 | 44 | 50 | 4 | - | 0-0-1- |
| | SaBE3 | AUGGUU | | | | | | | | | | | | | 3-8 |
| C325Y | EQR- | CUCUGGACACUGAC | 1763 | (CGAG) | 20 (C2/3) | 5.8 | 71 | 52 | 23 | 34 | 45 | 21 | 5 | - | 0-0-2- |
| | SpBE3 | UACACA | | | | | | | | | | | | | 14141 |
| P60L/S/F | SpBE3 | AACAGCUGCCCUUC | 1764 | (GGG) | 20 (C1/2) | 6.2 | 74 | 49 | 3 | 7 | 24 | 25 | 6 | - | 0-0-1- |
| | | AUCUAU | | | | | | | | | | | | | 10-115 |
| P111L/S/F | St1BE3 | GCUUUCUCCUUUCA | 1765 | (TAAGAAG) | 20 (C2/3) | 4.9 | 93 | 28 | 14 | 11 | 38 | 34 | 4 | + | 00 |
| | | GUCCUC | | | | | | | | | | | | | 5-29 |
| P187L/S/F | VQR- | CGUGACCCGUGGAA | 1766 | (GGAT) | 20 (C8-12) | 6.0 | 77 | 43 | 11 | 11 | 48 | 57 | 6 | + | 0-0-3- |
| | SpBE3 | CUGGCU | | | | | | | | | | | | | 9-35 |
| P744L/S/F | KKH- | AUGGAUCCUUUUGU | 1767 | (TGCAAT) | 20 (C14/15) | 6.6 | 91 | 28 | 0 | 0 | 38 | 59 | 6 | - | 0-0-0- |
| | SaBE3 | AGAUCU | | | | | | | | | | | | | 2-22 |
| P1722L/S/F | EQR- | AUCCUGGAAGUUCA | 1768 | (GGAG) | 20 (C9/10) | 4.7 | 63 | 55 | 53 | 16 | 23 | 44 | 4 | - | 0-0-3- |
| | SpBE3 | GUUGAA | | | | | | | | | | | | | 13-230 |
| G236R | SpBE3 | AAGCCCCUACAAUU | 1769 | (AGG) | 20 (C16/17) | 9.1 | 75 | 43 | 42 | 20 | 26 | 35 | 9 | - | 0-0-2- |
| | | GUCUUC | | | | | | | | | | | | | 9-84 |
| P1829L/S/F | VQR- | UGGAUCUGCCCAUG | 1770 | (GGTG) | 20 (C13/14) | 6.3 | 77 | 41 | 34 | 9 | 23 | 37 | 6 | - | 0-0-2- |
| | SpBE3 | GUUAGU | | | | | | | | | | | | | 11-102 |
| G1662R | VRER- | AAGUUGGACAUUCC | 1771 | (GGCG) | 20 (C12/13) | 4.2 | 64 | 54 | 19 | 8 | 66 | 59 | 4 | - | 0-1-1- |
| | SpBE3 | AAAGAU | | | | | | | | | | | | | 1-3 |
| P1133/5L/ | SpBE3 | GAUAACCCUUUGCC | 1772 | (AGG) | 20 (C10/11) | 3.7 | 66 | 51 | 42 | 12 | 82 | 33 | 3 | - | 0-0-1- |
| S/F | | UGGAGA | | | | | | | | | | | | | 15-115 |
| P1151L/S/F | KKH- | AUGAGCCAGAGGCC | 1773 | (ACAGAT) | 20 (C9110) | 6.0 | 93 | 24 | 18 | 1 | 16 | 51 | 6 | - | 0-0-0- |
| | SaBE3 | UGUUUC | | | | | | | | | | | | | 1-18 |
| P1722L/S/F | SpBE3 | CAUCCUGGAAGUUC | 1774 | (AGG) | 20 (C7/8) | 4.4 | 63 | 53 | 42 | 5 | 39 | 35 | 4 | - | 0-0-2- |
| | | AGUUGA | | | | | | | | | | | | | 21-161 |
| C134Y | VQR- | AGUGCACAUGAUGA | 1775 | (TGAA) | 20 (C2/3) | 6.3 | 55 | 60 | 55 | 16 | 61 | 29 | 6 | + | 0-1-2- |
| | SpBE3 | GCAUGC | | | | | | | | | | | | | 12-131 |
| C1562Y | SpBE3 | GCACACAUUCUCCA | 1776 | (AGG) | 20 (C12/13) | 6.0 | 45 | 47 | 69 | 21 | 61 | 24 | 6 | - | 0-1-2- |
| | | GUGAAA | | | | | | | | | | | | | 23-147 |
| C1159Y | SpBE3 | CCAUACACAACCUG | 1777 | (AAG) | 20 (C4/5) | 2.7 | 43 | 57 | 70 | 14 | 67 | 24 | 2 | - | 1-0-1- |
| | | ACAAGA | | | | | | | | | | | | | 8-88 |
| P906L/S/F | VQR- | UACGCUCCCACGGU | 1778 | (TGAA) | 20 (C10/11) | 6.6 | 47 | 53 | 59 | 10 | 44 | 46 | 6 | + | 1-0-0- |
| | SpBE3 | GGCACA | | | | | | | | | | | | | 2-33 |
| C315Y | SpBE3 | ACCACAAAGGAGAG | 1779 | (TGG) | 20 (C13/14) | 5.8 | 60 | 30 | 44 | 2 | 34 | 40 | 5 | - | 0-0-3- |
| | | CAUCUU | | | | | | | | | | | | | 19-168 |
| C1715Y | St1BE3 | ACAGUCGGGUGGCU | 1780 | (TAAGAAT) | 20 (C16/17) | 7.4 | 50 | 53 | 33 | 2 | 70 | 36 | 7 | - | 1-0-0- |
| | | UACUGU | | | | | | | | | | | | | 1-3 |
| P850L/S/F | VQR- | CUGGCCAACAUUGA | 1781 | (TGAT) | 200 () | 5.2 | 41 | 58 | 26 | 4 | 56 | 31 | 5 | - | 0-1-4- |
| | SpBE3 | ACAUGC | | | | | | | | | | | | | 23-152 |
| P1829L/S/F | SaBE3 | CUGCCCAUGGUUAG | 1782 | (CCGGAT) | 20 (C10-14) | 5.5 | 47 | 46 | 6 | 5 | 41 | 71 | 5 | - | 0-2-1- |
| | | UGGUGA | | | | | | | | | | | | | 1-13 |
| C275Y | SpBE3 | UUCGAAAACAUUUA | 1783 | (AGG) | 20 (C11/12) | 4.3 | 54 | 25 | 2 | 0 | 13 | 17 | 4 | - | 0-0-5- |
| | | UGCUUC | | | | | | | | | | | | | 15-166 |

^{a}BE types: SpBE3 = APOBEC1-SpCas9n-UGI; VQR-SpBE3 = APOBEC1-VQR-SpCas9n-UGI; EQR-SpBE3 = APOBEC1-EQR-SpCas9n-UGI; VRER-SpBE3 = APOBEC1-VRER-SpCas9n-UGI; SaBE3 = APOBEC1-SaCas9n-UGI; KKH-SaBE3 = APOBEC1-KKH-SaCas9n-UGI; St3BE3 = APOBEC1-St3Cas9n-UGI; St1BE3 = APOBEC1-St1Cas9n-UGI.
^{b}Efficiency score, based on Housden et al (Science Signaling, 2015, 8 (393):rs9).
^{c}Specificity scores based on Hsu et al (Nature biotechnology, 2013, 31 (9):827-832), Fusi et al (bioRxiv 021568; doi: http://dx.doi.org/10.1101/021568), Chari et al (Nature Methods, 2015, 12 (9):823-6), Doench et al (Nature Biotechnology, 2014, 32 (12):1262-7), Wang et al (Science, 2014, 343 (6166): 80-4), Moreno-Mateos et al (Nature Methods, 2015, 12 (10)982-8), Housden et al (Science Signaling, 2015, 8 (393):rs9), and the “Prox/GC” column shows “+” if the proximal 6 bp to the PAM has a GC count > = 4, and GG if the guide ends with GG, based on Farboud et al (Genetics, 2015, 199 (4):959-71).
^{d}Number of predicted off-target binding sites in the human genome allowing up to 0, 1, 2, 3 or 4 mismatches, respectively shown in the format 0-1-2-3-4. Algorithm used: Haeussler et al, Genome Biol. 2016; 17: 148.
^{d}Phospho-serine site S1490.

TABLE 9
Exemplary Efficiency and Specificity Scores for gRNAs for Alteration of Intron/Exon Junctions in NaV1.7 (SNA9A) Gene via
Base Editing

| | | Programmable | SEQ | | | | | | | | | | | Pro | |
|---|---|---|---|---|---|---|---|---|---|---|---|---|---|---|---|
| Target | BE | guide-RNA | ID | | gRNA size | | | | | | | M.- | Hous- | x/ | Off |
| variants | type^{a} | sequence | NOs | PAM | (C edited) | Eff^{b} | Hsu^{c} | Fusi | Chari | Doench | Wang | M. | den | GC | targets^{d} |
| acceptor, | KKH- | GCAGCACGCAGCGU | 1784 | (AAAAAT) | 20 (C15) | 6.6 | 98 | 60 | 99 | 52 | 91 | 67 | 6 | + | 0-0-0- |
| intron 9 | SaBE3 | CUAGGG | | | | | | | | | | | | | 0-8 |
| acceptor, | KKH- | GCAGCACGCAGCGU | 1785 | (AAT) | 20 (C15) | 6.6 | 98 | 60 | 99 | 52 | 91 | 67 | 6 | + | 0-0-0- |
| intron 9 | SaBE3 | CUAGGG | | | | | | | | | | | | | 0-8 |
| acceptor, | St3BE3 | GUUUAGGACCUAUA | 1786 | (TGGGG) | 20 (C10/11) | 5.6 | 97 | 71 | 97 | 40 | 89 | 30 | 5 | + | 0-0-0- |
| intron 16 | | UCAGGG | | | | | | | | | | | | | 0-18 |
| acceptor, | St1BE3 | GAUUCCAGUAAAGA | 1787 | (TGAGAAA) | 20 (C15/16) | 6.3 | 94 | 59 | 99 | 24 | 78 | 42 | 6 | - | 0-0-0- |
| intron 14 | | CCUAAG | | | | | | | | | | | | | 1-52 |
| donor, intron | KKH- | ACUUACAACUUGAA | 1788 | (ATAGGT) | 20 (C6) | 4.8 | 91 | 67 | 97 | 22 | 57 | 34 | 4 | + | 0-0-0- |
| 22 | SaBE3 | GCAGAG | | | | | | | | | | | | | 2-20 |
| acceptor, | St3BE3 | GCUAGAAACAUACC | 1789 | (TGGAG) | 20 (C14) | 4.6 | 96 | 58 | 90 | 19 | 53 | 44 | 4 | - | 0-0-0- |
| intron 26 | | UGUAUG | | | | | | | | | | | | | 1-19 |
| donor, intron | SaBE3 | ACCAGGGCACCACU | 1790 | (CAGGAT) | 20 (C2/3) | 8.4 | 86 | 70 | 97 | 37 | 55 | 49 | 8 | + | 0-0-2- |
| 19 | | GCUGAG | | | | | | | | | | | | | 7-16 |
| donor, intron | St1BE3 | AACCUCCAUACACA | 1791 | (CAAGAAA) | 20 (C16/17) | 3.4 | 98 | 61 | 84 | 25 | 77 | 29 | 3 | - | 0-0-0- |
| 19 | | ACCUGA | | | | | | | | | | | | | 0-10 |
| acceptor, | SpBE3 | GUUUAGGACCUAUA | 1792 | (TGG) | 20 (C10/11) | 5.6 | 84 | 71 | 97 | 40 | 89 | 30 | 5 | + | 0-0-1- |
| intron 16 | | UCAGGG | | | | | | | | | | | | | 6-63 |
| acceptor, | St1BE3 | AACGACCUAGUAUU | 1793 | (AAAGAAA) | 20 (C7) | 6.0 | 92 | 53 | 89 | 23 | 59 | 39 | 6 | - | 0-0-0- |
| intron 21 | | CAAAAG | | | | | | | | | | | | | 2-28 |
| donor, intron | KKH- | UUACGCAAAAACAA | 1794 | (CAAAAT) | 20 (C4) | 4.0 | 99 | 71 | 79 | 60 | 78 | 39 | 4 | - | 0-0-0- |
| 5 | SaBE3 | UGACGA | | | | | | | | | | | | | 0-10 |
| acceptor, | KKH- | CCAGUAAAGACCUA | 1795 | (AAAAAT) | 20 (C11/12) | 5.0 | 95 | 64 | 80 | 40 | 62 | 47 | 5 | - | 0-0-0- |
| intron 14 | SaBE3 | AGUGAG | | | | | | | | | | | | | 1-20 |
| acceptor, | SpBE3 | GACACUGACUACAC | 1796 | (AAG) | 20 (C9) | 4.0 | 87 | 60 | 87 | 17 | 87 | 30 | 4 | - | 0-0-1- |
| intron 8 | | ACGAGA | | | | | | | | | | | | | 5-60 |
| acceptor, | St1BE3 | UGUCUUCAGGCCUG | 1797 | (GGAGAAA) | 20 (C11/12) | 4.8 | 89 | 54 | 83 | 1 | 43 | 53 | 4 | - | 0-0-1- |
| intron 6 | | AAAAUG | | | | | | | | | | | | | 434 |
| donor, intron | VQR- | UUACCAGUCUGAAU | 1798 | (AGAA) | 20 (C4/5) | 6.6 | 92 | 62 | 80 | 68 | 64 | 12 | 6 | + | 0-0-0- |
| 15 | SpBE3 | GAUCGC | | | | | | | | | | | | | 4-58 |
| donor, intron | SpBE3 | CGUCCUUACGCUGU | 1799 | (AAG) | 20 (C9) | 7.5 | 84 | 58 | 85 | 20 | 53 | 57 | 7 | - | 0-0-0- |
| 12b | | CAUCAG | | | | | | | | | | | | | 449 |
| donor, intron | KKH- | ACUCGACAUUUUUG | 1800 | (TCCGGT) | 20 (C2) | 5.4 | 99 | 55 | 67 | 8 | 51 | 52 | 5 | + | 0-0-0- |
| 4 | SaBE3 | GUCCAG | | | | | | | | | | | | | 2-3 |
| acceptor, | VQR- | UUCCAGUAAAGACC | 1801 | (AGAA) | 20 (C13/14) | 2.7 | 74 | 68 | 91 | 13 | 62 | 33 | 2 | - | 0-0-1- |
| intron 14 | SpBE3 | UAAGUG | | | | | | | | | | | | | 9-206 |
| acceptor, | EQR- | GAUUCCAGUAAAGA | 1802 | (TGAG) | 20 (C13/14) | 6.3 | 63 | 59 | 99 | 24 | 78 | 42 | 6 | - | 0-0-1- |
| intron 14 | SpBE3 | CCUAAG | | | | | | | | | | | | | 28-179 |
| acceptor, | KKH- | GGUCGUGCCCUAAA | 1803 | (ATCAAT) | 20 (C9/10) | 6.1 | 85 | 32 | 76 | 59 | 69 | 25 | 6 | - | 0-0-0- |
| intron 12 | SaBE3 | AAAAAA | | | | | | | | | | | | | 1-14 |
| acceptor, | St3BE3 | UAGGUUUAGGACCU | 1804 | (GGGTG) | 20 (C13/14) | 6.1 | 97 | 64 | 28 | 11 | 64 | 52 | 6 | - | 0-0-0- |
| intron 16 | | AUAUCA | | | | | | | | | | | | | 0-13 |
| acceptor, | EQR- | GGACCUAUAUCAGG | 1805 | (AGAG) | 20 (C5/6) | 4.7 | 60 | 46 | 98 | 25 | 87 | 43 | 4 | + | 0-0-3- |
| intron 16 | SpBE3 | GuGGGG | | | | | | | | | | | | | 15-150 |
| acceptor, | VQR- | AGGUUUAGGACCUA | 1806 | (GGTG) | 20 (C12/13) | 5.1 | 79 | 71 | 79 | 5 | 75 | 50 | 5 | - | 0-0-1- |
| intron 16 | SpBE3 | UAUCAG | | | | | | | | | | | | | 5-100 |
| acceptor, | VQR- | ACACUGACUACACA | 1807 | (AGAA) | 20 (C8) | 3.4 | 82 | 59 | 76 | 25 | 72 | 42 | 3 | - | 0-0-0- |
| intron 8 | SpBE3 | cGAGAA | | | | | | | | | | | | | 10-130 |
| acceptor, | KKH- | GUAAAGACCUAAGU | 1808 | (AATAAT) | 20 (C8/9) | 6.9 | 75 | 50 | 82 | 55 | 84 | 26 | 6 | - | 0-0-1- |
| intron 14 | SaBE3 | GAGAAA | | | | | | | | | | | | | 7-59 |
| acceptor, | SpBE3 | ACGCAGCGUCUAGG | 1809 | (TGG) | 20 (C10) | 6.7 | 75 | 37 | 82 | 30 | 40 | 48 | 6 | - | 0-0-0- |
| intron 9 | | GAAAAA | | | | | | | | | | | | | 2-73 |
| donor, intron | VQR- | UUACUUGCAACCUA | 1810 | (CGAT) | 20 (C4) | 3.9 | 93 | 63 | 63 | 74 | 59 | 45 | 3 | + | 0-0-0- |
| 1 | SpBE3 | GCCCGC | 345 | | | | | | | | | | | | |
| donor, intron | KKH- | GGACACUUACAACU | 1811 | (AGAGAT) | 20 (C10) | 7.3 | 95 | 53 | 61 | 39 | 72 | -6 | 7 | - | 0-0-0- |
| 22 | SaBE3 | UGAAGC | | | | | | | | | | | | | 0-8 |
| acceptor, | KKH- | GAUUGGUCGUGCCC | 1812 | (AAAAAT) | 20 (C13/14) | 4.5 | 98 | 25 | 56 | 39 | 70 | 40 | 4 | - | 0-0-0- |
| intron 12 | SaBE3 | UAAAAA | | | | | | | | | | | | | 0-4 |
| acceptor, | VQR- | CUGGACACUGACUA | 1813 | (AGAA) | 20 (C12) | 5.5 | 85 | 69 | 69 | 75 | 67 | 61 | 5 | + | 0-0-1- |
| intron 8 | SpBE3 | CACACG | | | | | | | | | | | | | 7-86 |
| acceptor, | KKH- | AAACAUACCUGUAU | 1814 | (GAAAAT) | 20 (C9) | 6.2 | 90 | 63 | 62 | 15 | 70 | 43 | 6 | + | 0-0-0- |
| intron 26 | SaBE3 | GUGGAG | | | | | | | | | | | | | 1-20 |
| donor, intron | SpBE3 | UUUACCAGUCUGAA | 1815 | (CAG) | 20 (C5/6) | 6.4 | 83 | 69 | 70 | 50 | 57 | 26 | 6 | - | 0-0-2- |
| 15 | | UGAUCG | | | | | | | | | | | | | 3-86 |
| acceptor, | St3BE3 | ACCUAUAUCAGGGU | 1816 | (AGGGG) | 20 (C3/4) | 6.9 | 72 | 44 | 80 | 9 | 53 | 40 | 6 | + | 0-0-0- |
| intron 16 | | GGGGAG | | | | | | | | | | | | | 7-85 |
| acceptor, | St3BE3 | UUAGGACCUAUAUC | 1817 | (GGGAG) | 20 (C8/9) | 3.9 | 91 | 61 | 41 | 40 | 66 | 73 | 3 | + | 0-0-1- |
| intron 16 | | AGGGUG | | | | | | | | | | | | | 7-60 |
| acceptor, | St1BE3 | UCACAACGACCUAG | 1818 | (AAAGAAA) | 20 (C11) | 5.7 | 99 | 50 | 52 | 1 | 31 | 15 | 5 | - | 0-0-0- |
| intron 21 | | UAUUCA | | | | | | | | | | | | | 0-9 |
| acceptor, | KKH- | UUGUUCUGCAAAGA | 1819 | (AATAAT) | 20 (C6) | 5.6 | 67 | 43 | 84 | 47 | 61 | 41 | 5 | - | 0-0-2- |
| intron 25 | SaBE3 | AAUAAG | | | | | | | | | | | | | 9-62 |
| acceptor, | St1BE3 | CCUGUAUGUGGAGG | 1820 | (ATAGAAA) | 20 (C2) | 4.2 | 85 | 31 | 66 | 7 | 36 | 50 | 4 | - | 0-0-0- |
| intron 26 | | AAAAUA | | | | | | | | | | | | | 749 |
| donor, intron | St1BE3 | AAAACGUCCUUACG | 1821 | (TCAGAAG) | 20 (C13) | 3.6 | 98 | 53 | 27 | 2 | 65 | 30 | 3 | - | 0-0-0- |
| 12b | | CUGUCA | | | | | | | | | | | | | 1-8 |
| acceptor, | VQR- | UCUUCAGGCCUGAA | 1822 | (AGAA) | 20 (C9/10) | 6.6 | 56 | 67 | 94 | 74 | 77 | 54 | 6 | - | 0-0-2- |
| intron 6 | SpBE3 | AAUGGG | | | | | | | | | | | | | 23-213 |
| donor, intron | SaBE3 | UAUUUUUUUACCCC | 1823 | (AGGAAT) | 20 (C11/12) | 3.7 | 98 | 52 | 35 | 18 | 59 | 24 | 3 | + | 0-0-0- |
| 25 | | UGGUCG | | | | | | | | | | | | | 0-10 |
| donor, intron | KKH- | ACUUACUUGCAACC | 1824 | (GCCGAT) | 20 (C6) | 7.2 | 97 | 52 | 36 | 31 | 74 | 14 | 7 | + | 0-0-0- |
| 1 | SaBE3 | UAGCCC | | | | | | | | | | | | | 2-9 |
| donor, intron | VQR- | AUUUUUUUACCCCU | 1825 | (GGAA) | 20 (C10/11) | 8.0 | 83 | 60 | 66 | 49 | 47 | 32 | 8 | + | 0-0-1- |
| 25 | SpBE3 | GGUCGA | | | | | | | | | | | | | 12-133 |
| acceptor, | VQR- | UACUAUGAAAGUCU | 1826 | (GGAA) | 20 (C13) | 5.3 | 62 | 63 | 86 | 28 | 64 | 37 | 5 | + | 0-0-5- |
| intron 2 | SpBE3 | GCAGGA | | | | | | | | | | | | | 14-194 |
| donor, intron | VQR- | AAUAUUCUUACCUA | 1827 | (AGAT) | 20 (C11/12) | 4.2 | 63 | 62 | 85 | 15 | 80 | 51 | 4 | - | 0-0-2- |
| 26 | SpBE3 | CAAUGG | | | | | | | | | | | | | 22-264 |
| acceptor, | SaBE3 | ACCUAAACACAAGA | 1828 | (TGGGAT) | 20 (C2/3) | 6.7 | 91 | 56 | 28 | 11 | 58 | 26 | 6 | - | 0-0-1- |
| intron 4 | | UUCCAU | | | | | | | | | | | | | 1-18 |
| acceptor, | KKH- | AGAGGCCUGGAUGG | 1829 | (AGAAAT) | 20 (C6/7) | 6.8 | 80 | 60 | 66 | 52 | 81 | 75 | 6 | - | 0-0-3- |
| intron 1 | SaBE3 | AAACAA | | | | | | | | | | | | | 3-20 |
| donor, intron | KKH- | AGUACCUACAUCAA | 1830 | (GGAAAT) | 20 (C9) | 4.6 | 87 | 41 | 59 | 9 | 46 | 33 | 4 | - | 0-0-1- |
| 20 | SaBE3 | CAAUUA | | | | | | | | | | | | | 4-23 |
| donor, intron | SpBE3 | GACACUUACAACUU | 1831 | (GAG) | 20 (C9) | 5.3 | 62 | 63 | 83 | 47 | 76 | 33 | 5 | - | 0-0-4- |
| 22 | | GAAGCA | | | | | | | | | | | | | 13-121 |
| acceptor, | SpBE3 | CCAUACACAACCUG | 1832 | (AAG) | 20 (C11/12) | 2.7 | 74 | 57 | 70 | 14 | 67 | 24 | 2 | - | 0-0-1- |
| intron 18 | | ACAAGA | | | | | | | | | | | | | 8-88 |
| acceptor, | KKH- | CGCAGCGUCUAGGG | 1833 | (GGAAAT) | 20 (C9) | 6.0 | 98 | 44 | 17 | 28 | 29 | 52 | 6 | - | 0-0-0- |
| intron 9 | SaBE3 | AAAAAU | | | | | | | | | | | | | 0-2 |
| donor, intron | SpBE3 | GAGACUUACCAAAU | 1834 | (TAG) | 20 (C5/6) | 7.4 | 67 | 49 | 75 | 14 | 77 | 32 | 7 | - | 0-0-1- |
| 14 | | UUCCUA | | | | | | | | | | | | | 18-121 |
| acceptor, | St3BE3 | UUGUCUUCAGGCCU | 1835 | (GGGAG) | 20 (C12/13) | 5.8 | 92 | 49 | 33 | 25 | 32 | 59 | 5 | - | 0-0-1- |
| intron 6 | | GAAAAU | | | | | | | | | | | | | 3-22 |
| donor, intron | KKH- | UUCUUACCUGGGAU | 1836 | (AATAGT) | 20 (C7/8) | 5.3 | 89 | 52 | 37 | 17 | 49 | 45 | 5 | - | 0-0-1- |
| 6 | SaBE3 | UACAGA | | | | | | | | | | | | | 2-14 |
| acceptor, | SpBE3 | UUUAGGACCUAUAU | 1837 | (GGG) | 20 (C9/10) | 7.5 | 80 | 60 | 15 | 17 | 69 | 48 | 7 | + | 0-0-1- |
| intron 16 | | CAGGGU | | | | | | | | | | | | | 11-87 |
| donor, intron | KKH- | AACGUCCUUACGCU | 1838 | (AGAAGT) | 20 (C11) | 6.4 | 95 | 45 | 34 | 4 | 28 | 46 | 6 | - | 0-0-0- |
| 12b | SaBE3 | GUCAUC | | | | | | | | | | | | | 1-4 |
| acceptor, | SpBE3 | AGGACCUAUAUCAG | 1839 | (GAG) | 20 (C6/7) | 5.1 | 50 | 53 | 89 | 8 | 64 | 72 | 5 | + | 0-0-3- |
| intron 16 | | GGUGGG | | | | | | | | | | | | | 23-231 |
| acceptor, | SpBE3 | UUAGGACCUAUAUC | 1840 | (GGG) | 20 (C8/9) | 3.9 | 77 | 61 | 41 | 40 | 66 | 73 | 3 | + | 0-0-1- |
| intron 16 | | AGGGUG | | | | | | | | | | | | | 19-153 |
| acceptor, | EQR- | UGUCUUCAGGCCUG | 1841 | (GGAG) | 20 (C11/12) | 4.8 | 55 | 54 | 83 | 1 | 43 | 53 | 4 | - | 0-0-3- |
| intron 6 | SpBE3 | AAAAUG | | | | | | | | | | | | | 26-246 |
| donor, intron | St3BE3 | AUAAAUAUUCUUAC | 1842 | (TGGAG) | 20 (C14/15) | 5.7 | 88 | 50 | 27 | 15 | 59 | 35 | 5 | - | 0-0-1- |
| 26 | | CUACAA | | | | | | | | | | | | | 5-55 |
| acceptor, | KKH- | CAUACCUGUAUGUG | 1843 | (AATAAT) | 20 (C6) | 4.3 | 81 | 56 | 50 | 6 | 48 | 42 | 4 | - | 0-0-0- |
| intron 26 | SaBE3 | GAGGAA | | | | | | | | | | | | | 1-28 |
| donor, intron | St1BE3 | GUUUACCAGUCUGA | 1844 | (GCAGAAC) | 20 (C6/7) | 6.1 | 94 | 43 | 23 | 1 | 42 | 38 | 6 | - | 0-0-0- |
| 15 | | AUGAUC | | | | | | | | | | | | | 3-14 |
| donor, ntron | VQR- | GAUUAUUACAUACC | 1845 | (AGTG) | 20 (C13/14) | 3.4 | 76 | 55 | 60 | 7 | 73 | 28 | 3 | - | 0-0-0- |
| 13 | SpBE3 | UUCCAC | | | | | | | | | | | | | 7-137 |
| donor, intron | SpBE3 | UCACCUUUUUGUCU | 1846 | (TAG) | 20 (C4/5) | 5.9 | 65 | 45 | 71 | 6 | 42 | 16 | 5 | - | 0-0-1- |
| 2 | | GCAUAG | | | | | | | | | | | | | 21-176 |
| acceptor, | St1BE3 | AUGAAAGUCUGCAG | 1847 | (AAAGAAA) | 20 (C9) | 5.0 | 77 | 52 | 58 | 14 | 63 | 44 | 5 | - | 0-0-1- |
| intron 2 | | GAGGAA | | | | | | | | | | | | | 4-118 |
| donor, intron | SpBE3 | AAACGUCCUUACGC | 1848 | (CAG) | 20 (C12) | 5.6 | 82 | 53 | 14 | 14 | 46 | 62 | 5 | - | 0-0-1- |
| 12b | | UGUCAU | | | | | | | | | | | | | 14-25 |
| donor, intron | KKH- | CACUUACUCGACAU | 1849 | (TCCAGT) | 20 (C7) | 6.5 | 91 | 44 | 39 | 51 | 51 | 39 | 6 | - | 0-0-0- |
| 4 | SaBE3 | UUUUGG | | | | | | | | | | | | | 1-7 |
| donor, intron | KKH- | CUUGGUACUCACCU | 1850 | (AAAGGT) | 20 (C12/13) | 9.4 | 92 | 43 | 29 | 31 | 42 | 39 | 9 | - | 0-0-2- |
| 9 | SaBE3 | GUUGGU | | | | | | | | | | | | | 1-11 |
| acceptor, | VQR- | GCAAUUGCCUGGUU | 1851 | (AGAC) | 20 (C8/9) | 5.6 | 79 | 54 | 55 | 36 | 82 | 49 | 5 | + | 0-0-2- |
| intron 10 | SpBE3 | GGGCCA | | | | | | | | | | | | | 9-83 |
| donor, intron | VQR- | AACGUCCUUACGCU | 1852 | (AGAA) | 20 (C11) | 6.4 | 89 | 45 | 34 | 4 | 28 | 46 | 6 | - | 0-0-0- |
| 12b | SpBE3 | GUCAUC | | | | | | | | | | | | | 2-33 |
| acceptor, | VQR- | CAUACACAACCUGA | 1853 | (AGAC) | 20 (C10/11) | 7.1 | 50 | 54 | 84 | 9 | 48 | 51 | 7 | - | 0-0-5- |
| intron 18 | SpBE3 | CAAGAA | | | | | | | | | | | | | 39-478 |
| acceptor, | St1BE3 | AAGUUCUGGGAGAA | 1854 | (AGAGAAC) | 20 (C6) | 4.9 | 81 | 52 | 40 | 6 | 73 | 43 | 4 | - | 0-0-0- |
| intron 13 | | AAAAGC | | | | | | | | | | | | | 13-84 |
| donor, intron | KKH- | UGGUUACAUACCAC | 1855 | (TCCAAT) | 20 (C12) | 4.5 | 91 | 42 | 10 | 3 | 43 | 55 | 4 | - | 0-0-0- |
| 16 | SaBE3 | CAGGUU | | | | | | | | | | | | | 3-12 |
| acceptor, | St1BE3 | UGCCUUUAAGAAUA | 1856 | (ATAGAAT) | 20 (C3/4) | 2.9 | 84 | 21 | 48 | 4 | 33 | 14 | 2 | - | 0-0-0- |
| intron 22 | | ACAUUA | | | | | | | | | | | | | 7-68 |
| acceptor, | St1BE3 | UAAGAGGCCUGGAU | 1857 | (AAAGAAA) | 20 (C8/9) | 6.2 | 93 | 36 | 37 | 6 | 53 | 50 | 6 | - | 0-0-0- |
| intron 1 | | GGAAAC | | | | | | | | | | | | 1 | -38 |
| donor, intron | EQR- | GGACACUUACAACU | 1858 | (AGAG) | 20 (C9) | 7.3 | 69 | 53 | 61 | 39 | 72 | -6 | 7 | - | 0-0-2- |
| 22 | SpBE3 | UGAAGC | | | | | | | | | | | | | 11-129 |
| donor, intron | SpBE3 | UAUUUUUUUACCCC | 1859 | (AGG) | 20 (C11/12) | 3.7 | 78 | 52 | 35 | 18 | 59 | 24 | 3 | + | 0-0-1- |
| 25 | | UGGUCG | | | | | | | | | | | | | 12-87 |
| donor, intron | KKH- | AUACCCACUUACUC | 1860 | (TTTGGT) | 20 (C12) | 5.4 | 99 | 30 | 18 | 16 | 27 | 33 | 5 | - | 0-0-0- |
| 4 | SaBE3 | GACAUU | | | | | | | | | | | | | 0-4 |
| donor, intron | SpBE3 | CUUACUCGACAUUU | 1861 | (CAG) | 20 (C5) | 8.7 | 80 | 48 | 9 | 12 | 32 | 31 | 8 | - | 0-0-0- |
| 4 | | UUGGUC | | | | | | | | | | | | | 5-56 |
| donor, intron | SpBE3 | UGGUACUCACCUGU | 1862 | (AGG) | 20 (C10/11) | 8.3 | 82 | 46 | 2 | 20 | 44 | 52 | 8 | - | 0-0-1- |
| 9 | | UGGUAA | | | | | | | | | | | | | 9-84 |
| acceptor, | SaBE3 | AAUAGGUUUAGGAC | 1863 | (CAGGGT) | 20 (C15/16) | 4.3 | 91 | 36 | 7 | 5 | 43 | 34 | 4 | - | 0-0-0- |
| intron 16 | | CUAUAU | | | | | | | | | | | | | 6-11 |
| acceptor, | VQR- | ACAACGACCUAGUA | 1864 | (AGAA) | 20 (C9) | 4.1 | 76 | 51 | 50 | 41 | 66 | 31 | 4 | - | 0-0-0- |
| intron 21 | SpBE3 | UUCAAA | | | | | | | | | | | | | 4-113 |
| acceptor, | SaBE3 | GCCUUUAAGAAUAA | 1865 | (TAGAAT) | 20 (C2/3) | 5.9 | 89 | 24 | 38 | 3 | 42 | 26 | 5 | - | 0-0-0- |
| intron 22 | | CAUUAA | | | | | | | | | | | | | 1-38 |
| donor, intron | SpBE3 | GGGUGGUACCUGAU | 1866 | (TAG) | 20 (C9/10) | 7.7 | 70 | 47 | 57 | 17 | 90 | 60 | 7 | + | 0-0-2- |
| 11 | | UGGGGG | | | | | | | | | | | | | 12-152 |
| acceptor, | SpBE3 | ACGACCUAGUAUUC | 1867 | (AAG) | 20 (C6) | 4.9 | 72 | 54 | 45 | 16 | 69 | 33 | 4 | - | 0-0-1- |
| intron 21 | | AAAAGA | | | | | | | | | | | | | 6-79 |
| acceptor, | VQR- | GAAACAUACCUGUA | 1868 | (GGAA) | 20 (C10) | 4.9 | 63 | 63 | 60 | 33 | 66 | 45 | 4 | - | 0-0-0- |
| intron 26 | SpBE3 | UGUGGA | | | | | | | | | | | | | 21-206 |
| donor, intron | KKH- | UAAACUCACCUUUU | 1869 | (CATAGT) | 20 (C9/10) | 8.0 | 57 | 57 | 68 | 6 | 42 | 37 | 8 | - | 0-0-1- |
| 2 | SaBE3 | UGUCUG | | | | | | | | | | | | | 5-21 |
| donor, intron | VQR- | UACAUACCCUGAAU | 1870 | (TGAA) | 20 (C7/8) | 5.8 | 75 | 50 | 8 | 6 | 62 | 12 | 5 | + | 0-0-0- |
| 8 | SpBE3 | CUGUGC | | | | | | | | | | | | | 15-118 |
| acceptor, | EQR- | CUAGAAACAUACCU | 1871 | (GGAG) | 20 (C13) | 6.2 | 66 | 58 | 16 | 18 | 50 | 34 | 6 | - | 0-0-0- |
| intron 26 | SpBE3 | GUAUGU | | | | | | | | | | | | | 19-215 |
| acceptor, | VQR- | CGACCUAGUAUUCA | 1872 | (AGAA) | 20 (C5) | 3.6 | 59 | 51 | 64 | 20 | 65 | 43 | 3 | - | 0-0-1- |
| intron 21 | SpBE3 | AAAGAA | | | | | | | | | | | | | 10-143 |
| acceptor, | KKH- | AAGUUGCCUUUAAG | 1873 | (ATTAAT) | 20 (C7/8) | 4.4 | 87 | 36 | 23 | 11 | 39 | 38 | 4 | - | 0-0-0- |
| intron 22 | SaBE3 | AAUAAC | | | | | | | | | | | | | 735 |
| donor, intron | VQR- | GGUGGUACCUGAUU | 1874 | (AGAC) | 20 (C8/9) | 6.8 | 73 | 50 | 43 | 22 | 78 | 63 | 6 | + | 0-0-5- |
| 11 | SpBE3 | GGGGGu | | | | | | | | | | | | | 13-84 |
| donor, intron | St3BE3 | CACCUUUUUGUCUG | 1875 | (AGGGG) | 20 (C3/4) | 3.8 | 91 | 32 | 7 | 1 | 13 | 41 | 3 | - | 0-0-0- |
| 2 | | CAUAGU | | | | | | | | | | | | | 2-31 |
| acceptor, | SpBE3 | ACCUAAACACAAGA | 1876 | (TGG) | 20 (C2/3) | 6.7 | 66 | 56 | 28 | 11 | 58 | 26 | 6 | - | 0-0-3- |
| intron 4 | | UUCCAU | | | | | | | | | | | | | 17-119 |
| acceptor, | VQR- | CGCAGCGUCUAGGG | 1877 | (GGAA) | 20 (C9) | 6.0 | 78 | 44 | 17 | 28 | 29 | 52 | 6 | - | 0-0-0- |
| intron 9 | SpBE3 | AAAAAU | | | | | | | | | | | | | 4-61 |
| donor, intron | St3BE3 | AAUUUGGGUGGUAC | 1878 | (GGGGG) | 20 (C14/15) | 5.3 | 96 | 26 | 9 | 2 | 36 | 67 | 5 | - | 0-0-0- |
| 11 | | CUGAUU | | | | | | | | | | | | | 3-31 |
| donor, intron | SpBE3 | CCUUUUUGUCUGCA | 1879 | (GGG) | 20 (C1/2) | 6.0 | 66 | 56 | 38 | 61 | 59 | 37 | 6 | - | 0-0-3- |
| 2 | | UAGUAG | | | | | | | | | | | | | 22-194 |
| donor, intron | SpBE3 | UUUACCCCUGGUCG | 1880 | (TGG) | 20 (C5/6) | 5.1 | 89 | 33 | 8 | 13 | 26 | 52 | 5 | - | 0-0-0- |
| 25 | | AGGAAU | | | | | | | | | | | | | 8-52 |
| donor, intron | VQR- | UACUUACGCAAAAA | 1881 | (CGAC) | 20 (C7) | 4.1 | 56 | 62 | 66 | 22 | 75 | 29 | 4 | - | 0-0-2- |
| 5 | SpBE3 | CAAUGA | | | | | | | | | | | | | 14-257 |
| acceptor, | SaBE3 | CCUAUAUCAGGGUG | 1882 | (GGGGGT) | 20 (C2/3) | 3.8 | 76 | 45 | 25 | 4 | 47 | 56 | 3 | + | 0-0-0- |
| intron 16 | | GGGAGA | | | | | | | | | | | | | 1-30 |
| donor, intron | SaBE3 | AAUUUGGGUGGUAC | 1883 | (GGGGGT) | 20 (C14/15) | 5.3 | 95 | 26 | 9 | 2 | 36 | 67 | 5 | - | 0-0-0- |
| 11 | | CUGAUU | | | | | | | | | | | | | 2-5 |
| donor, intron | SpBE3 | UUGGUACUCACCUG | 1884 | (AAG) | 20 (C11/12) | 3.7 | 83 | 38 | 12 | 0 | 37 | 54 | 3 | - | 0-0-1- |
| 9 | | UUGGUA | | | | | | | | | | | | | |
| donor, intron | EQR- | AUUUACCAGGGCAC | 1885 | (TGAG) | 20 (C6/7) | 6.4 | 67 | 40 | 53 | 1 | 55 | 10 | 6 | + | 0-0-3- |
| 19 | SpBE3 | CACUGC | | | | | | | | | | | | | 22-108 |
| donor, intron | KKH- | ACAUACCUUCCACA | 1886 | (GTTAAT) | 20 (C6/7) | 6.3 | 79 | 41 | 30 | 13 | 33 | 31 | 6 | - | 0-0-0- |
| 13 | SaBE3 | GUGUUU | | | | | | | | | | | | | 3-38 |
| donor, intron | KKH- | CACCUGUUGGUAAA | 1887 | (CCCAGT) | 20 (C3/4) | 5.2 | 93 | 26 | 7 | 3 | 7 | 27 | 5 | - | 0-0-0- |
| 9 | SaBE3 | GGUUUU | | | | | | | | | | | | | 1-14 |
| donor, intron | KKH- | GACUUACCAAAUUU | 1888 | (GCAAGT) | 20 (C7/8) | 6.2 | 83 | 35 | 29 | 15 | 36 | 52 | 6 | - | 0-0-0- |
| 14 | SaBE3 | CCUAUA | | | | | | | | | | | | | 4-31 |
| donor, intron | VQR- | ACACUUACAACUUG | 1889 | (AGAT) | 20 (C8) | 3.5 | 55 | 63 | 62 | 38 | 87 | 45 | 3 | - | 0-0-9- |
| 22 | SpBE3 | AAGCAG | | | | | | | | | | | | | 16-173 |
| donor, intron | St1BE3 | AUUACUUCUUACCU | 1890 | (ACAGAAA) | 20 (C12/13) | 7.0 | 91 | 27 | 13 | 3 | 29 | 37 | 7 | - | 0-0-1- |
| 6 | | GGGAUU | | | | | | | | | | | | | 4-30 |
| acceptor, | SpBE3 | UGCAAUUGCCUGGU | 1891 | (AAG) | 20 (C9/10) | 7.8 | 74 | 43 | 11 | 8 | 40 | 37 | 7 | + | 0-0-1- |
| intron 10 | | UGGGCC | | | | | | | | | | | | | 15-146 |
| acceptor, | SpBE3 | CGGAGCUAAAAGCA | 1892 | (AAG) | 20 (C6) | 4.6 | 65 | 35 | 52 | 11 | 36 | 46 | 4 | - | 0-0-2- |
| intron 15 | | AAUAUA | | | | | | | | | | | | | 16-105 |
| acceptor, | KKH- | CUCGGAGCUAAAAG | 1893 | (TAAAGT) | 20 (C8) | 4.8 | 88 | 28 | 29 | 7 | 33 | 42 | 4 | - | 0-0-0- |
| intron 15 | SaBE3 | CAAAUA | | | | | | | | | | | | | 1-16 |
| donor, intron | SaBE3 | CACCUUUUUGUCUG | 1894 | (AGGGGT) | 20 (C3/4) | 3.8 | 84 | 32 | 7 | 1 | 13 | 41 | 3 | - | 0-0-1- |
| 2 | | CAUAGU | | | | | | | | | | | | | 15-17 |
| donor, intron | SpBE3 | AACUCACCUUUUUG | 1895 | (TAG) | 20 (C7/8) | 3.7 | 65 | 49 | 12 | 13 | 29 | 20 | 3 | - | 0-0-0- |
| 2 | | UCUGCA | | | | | | | | | | | | | 20-206 |
| acceptor, | VQR- | AGAGGCCUGGAUGG | 1896 | (AGAA) | 20 (C6/7) | 6.8 | 46 | 60 | 66 | 52 | 81 | 75 | 6 | - | 0-0-5- |
| intron 1 | SpBE3 | AAACAA | | | | | | | | | | | | | 54-404 |
| donor, intron | VQR- | AGUACCUACAUCAA | 1897 | (GGAA) | 20 (C9) | 4.6 | 52 | 41 | 59 | 9 | 46 | 33 | 4 | - | 0-0-3- |
| 20 | SpBE3 | CAAUUA | | | | | | | | | | | | | 53-739 |
| acceptor, | SaBE3 | AUAAAAAUAUUCUG | 1898 | (AAGAAT) | 20 (C12) | 3.8 | 58 | 53 | 50 | 8 | 53 | 22 | 3 | - | 0-0-8- |
| intron 7 | | UUGAAG | | | | | | | | | | | | | 13-102 |
| acceptor, | St3BE3 | CCUAUAUCAGGGUG | 1899 | (GGGGG) | 20 (C2/3) | 3.8 | 64 | 45 | 25 | 4 | 47 | 56 | 3 | + | 0-0-0- |
| intron 16 | | GGGAGA | | | | | | | | | | | | | 12-144 |
| acceptor, | KKH- | UGUCUACCUAUAAA | 1900 | (AAAAGT) | 20 (C7/8) | 4.3 | 78 | 31 | 9 | 17 | 26 | 16 | 4 | - | 0-0-0- |
| intron 23 | SaBE3 | AUUUAC | | | | | | | | | | | | | 3-55 |
| acceptor, | St1BE3 | UAAAUACCUGUAGA | 1901 | (TCAGAAT) | 20 (C7/8) | 4.1 | 68 | 16 | 41 | 10 | 40 | 39 | 4 | - | 0-0-1- |
| intron 5 | | AUUAAA | | | | | | | | | | | | | 17-167 |
| donor, intron | SpBE3 | ACCCACUUACUCGA | 1902 | (TGG) | 20 (C10) | 7.1 | 86 | 20 | 8 | 4 | 7 | 12 | 7 | - | 0-0-1- |
| 4 | | CAUUUU | | | | | | | | | | | | | 4-58 |
| acceptor, | St1BE3 | GAUUUUGUUCUGCA | 1903 | (TAAGAAT) | 20 (C10) | 5.9 | 68 | 35 | 37 | 15 | 76 | 33 | 5 | - | 0-0-3- |
| intron 25 | | AAGAAA | | | | | | | | | | | | | 16-104 |
| donor, intron | KKH- | UUACCCUCAUUCCU | 1904 | (CTAGAT) | 20 (C4/5) | 5.2 | 64 | 40 | 20 | 51 | 30 | 21 | 5 | - | 0-1-1- |
| 21 | SaBE3 | UCAAAU | | | | | | | | | | | | | 3-19 |
| donor, intron | EQR- | UAUAUUUUUUUACC | 1905 | (CGAG) | 20 (C13/14) | 4.1 | 51 | 53 | 36 | 12 | 37 | 25 | 4 | + | 0-0-3- |
| 25 | SpBE3 | ccuGGu | | | | | | | | | | | | | 38-413 |
| donor, intron | KKH- | UAGUACACUCAUAU | 1906 | (AAAAAT) | 20 (C8/10) | 9.1 | 90 | 10 | 4 | 4 | 18 | 42 | 9 | - | 0-0-0- |
| 3 | SaBE3 | ccuuuu | | | | | | | | | | | | | 2-26 |
| donor, intron | VQR- | ACCCUCAUUCCUUC | 1907 | (AGAT) | 20 (C2/3) | 5.6 | 62 | 36 | 3 | 1 | 28 | 12 | 5 | - | 0-0-4- |
| 21 | SpBE3 | AAAUCU | | | | | | | | | | | | | 22-199 |
| donor, intron | VQR- | AAUAUUACAUACCC | 1908 | (TGTG) | 20 (C12/13) | 4.5 | 63 | 31 | 16 | 2 | 39 | 12 | 4 | - | 0-0-2- |
| 8 | SpBE3 | UGAAUC | | | | | | | | | | | | | 13-223 |
| donor, intron | SpBE3 | AAGUACCUACAUCA | 1909 | (AGG) | 20 (C10) | 6.6 | 53 | 40 | 36 | 16 | 43 | 42 | 6 | - | 0-0-8- |
| 20 | | ACAAUU | | | | | | | | | | | | | 38-315 |
| donor, intron | SpBE3 | CACCUUUUUGUCUG | 1910 | (AGG) | 20 (C3/4) | 3.8 | 60 | 32 | 7 | 1 | 13 | 41 | 3 | - | 0-0-3- |
| 2 | | CAUAGU | | | | | | | | | | | | | 25-159 |
| donor, intron | SpBE3 | UUACAACUUGAAGC | 1911 | (AGG) | 20 (C4) | 4.6 | 41 | 47 | 37 | 45 | 44 | 56 | 4 | - | 0-0-10- |
| 22 | | AGAGAU | | | | | | | | | | | | | 36-283 |

^{a}BE types: SpBE3 = APOBEC1-SpCas9n-UGI; VQR-SpBE3 = APOBEC1-VQR-SpCas9n-UGI; EQR-SpBE3 = APOBEC1-EQR-SpCas9n-UGI; VRER-SpBE3 = APOBEC1-VRER-SpCas9n-UGI; SaBE3 = APOBEC1-SaCas9n-UGI; KKH-SaBE3 = APOBEC1-KKH-SaCas9n-UGI; St3BE3 = APOBEC1-St3Cas9n-UGI; St1BE3 = APOBEC1-St1Cas9n-UGI.
^{b}Efficiency score, based on Housden et al (Science Signaling, 2015, 8 (393):rs9).
^{c}Specificity scores based on Hsu et al (Nature biotechnology, 2013, 31 (9):827-832), Fusi et al (bioRxiv 021568; doi: http://dx.doi.org/10.1101/021568), Chari et al (Nature Methods, 2015, 12 (9):823-6), Doench et al (Nature Biotechnology, 2014, 32 (12):1262-7), Wang et al (Science, 2014, 343 (6166): 80-4), Moreno-Mateos et al (Nature Methods, 2015, 12 (10)982-8), Housden et al (Science Signaling, 2015, 8 (393):rs9), and the “Prox/GC” column shows “+” if the proximal 6 bp to the PAM has a GC count > = 4, and GG if the guide ends with GG, based on Farboud et al (Genetics, 2015, 199 (4):959-71).
^{d}Number of predicted off-target binding sites in the human genome allowing up to 0, 1, 2, 3 or 4 mismatches, respectively shown in the format 0-1-2-3-4. Algorithm used: Haeussler et al, Genome Biol. 2016; 17: 148. Isoform 2 is expressed preferentially in the dorsal root ganglion.

### Editing the SCN9A Gene Using Cas9 Nuclease or Cas9 Nickase Pairs

In some embodiments, the editing of an ion channel-encoding polynucleotide (e.g., SCN9A gene) may be achieved using Cas9 nucleases, or Cas9 nickase pairs (e.g., as described in Ran et al., Cell. 2013 Sep. 12; 154(6): 1380-1389, incorporated herein by reference. Cas9 nuclease or Cas9 nickase pairs introduce double stranded DNA break in the ion channel-encoding polynucleotide (e.g., SCN9A gene). Indels may be introduced when the double strand break is repaired by the cellular double strand break repair system, causing loss-of-function SCN9A mutants. The use of Cas9 nuclease to generate SCN9A mutation have been described in the art, e.g., in Sun et al., Transl Perioper Pain Med. 2016; 1(3): 22-33, incorporated herein by reference.

Nonetheless, provided herein are top-scoring guide-RNA target sites in SCN9A gene using these alternative genome editing agents (Table 10 and Table 11).

TABLE 10
Top-Scoring Guide-RNA Target Sites and PAM Sequences in SCN9A for
Cas9 Nuclease

| | | | Guide- | SEQ ID | Specificity | Efficiency |
|---|---|---|---|---|---|---|
| Entry | Position | Strand | RNA target site (PAM) | NOs | score⁵⁰ | score²⁸ |
| 1 | 350 | -1 | GAGCACGGGCGAAAGACCGA(GGG) | 1912 | 94 | 67 |
| 2 | 439 | 1 | GTATTACGCCACCTGGAAAG(AAG) | 1913 | 81 | 70 |
| 3 | 532 | -1 | ACAGAGTCAAAACCGCACAG(GAG) | 1914 | 84 | 89 |
| 4 | 534 | -1 | CCACAGAGTCAAAACCGCAC(AGG) | 1915 | 84 | 60 |
| 5 | 753 | 1 | AGCTTAGCAGATACAACCTG(TGG) | 1916 | 60 | 72 |
| 6 | 755 | 1 | CTTAGCAGATACAACCTGTG(GGG) | 1917 | 70 | 73 |
| 7 | 837 | 1 | TCTGCCCCTATTTCTCAGCG(CAG) | 1918 | 75 | 83 |
| 8 | 1555 | -1 | TCATGAAAATTTGCGACACA(GGG) | 1919 | 84 | 66 |
| 9 | 2064 | -1 | CTACTTTTTTCCTTGCCACA(GAG) | 1920 | 51 | 81 |
| 10 | 2380 | -1 | GCTGAAATGGAGTAATAAGG(AAG) | 1921 | 61 | 85 |
| 11 | 2596 | -1 | ATAGAGAATGAATTGCAGGG(GAG) | 1922 | 52 | 87 |
| 12 | 2846 | -1 | ATGTGTTTTAGCCACGACCT(GGG) | 1923 | 90 | 62 |
| 13 | 3685 | -1 | AAACATCAATTTAGACCGTG(TGG) | 1924 | 83 | 64 |
| 14 | 5589 | -1 | AAAACATTAGCCGGGCACGG(TGG) | 1925 | 78 | 71 |
| 15 | 5724 | -1 | AGATAATGGGCTGAGCGCGG(TGG) | 1926 | 88 | 65 |
| 16 | 6504 | 1 | GGCCTACTCAGGGATCAACT(GGG) | 1927 | 82 | 66 |
| 17 | 7409 | 1 | GGAGTGCAGTGGTACGATGT(TGG) | 1928 | 89 | 62 |
| 18 | 7790 | -1 | TGGTCATGAGGATTTAAACG(GAG) | 1929 | 77 | 77 |
| 19 | 7963 | 1 | CCCCACACAGATATACCTGG(TGG) | 1930 | 69 | 76 |
| 20 | 7967 | -1 | TGCTTGGTAGCGTAACCACC(AGG) | 1931 | 92 | 61 |
| 21 | 8465 | 1 | ACGCCCGTAATCCAGCACTT(TGG) | 1932 | 89 | 67 |
| 22 | 10500 | 1 | GGGATTACTAACCTGCGTCG(AGG) | 1933 | 95 | 65 |
| 23 | 10501 | 1 | GGATTACTAACCTGCGTCGA(GGG) | 1934 | 98 | 61 |
| 24 | 10501 | -1 | TTATCACGCAGCCCTCGACG(CAG) | 1935 | 96 | 71 |
| 25 | 11027 | 1 | CTAGCAAAACAGATACCAAG(GAG) | 1936 | 59 | 81 |
| 26 | 11046 | 1 | GGAGACACCGCATGTTGTCA(GGG) | 1937 | 83 | 62 |
| 27 | 11105 | -1 | GTGTTTTGAGATCCGTAAGG(CAG) | 1938 | 90 | 71 |
| 28 | 11108 | -1 | AGCGTGTTTTGAGATCCGTA(AGG) | 1939 | 91 | 62 |
| 29 | 11121 | 1 | TTACGGATCTCAAAACACGC(TAG) | 1940 | 92 | 86 |
| 30 | 11466 | -1 | TTCTGATATATGCTACGACC(CGG) | 1941 | 92 | 60 |
| 31 | 12273 | -1 | TGAATCACAGACCTAAACGT(CAG) | 1942 | 79 | 79 |
| 32 | 12530 | -1 | GCAAGGATATTCTTTCCCAT(CAG) | 1943 | 59 | 81 |
| 33 | 12956 | -1 | TAGGGGACTTAACCTCCACA(AGG) | 1944 | 81 | 67 |
| 34 | 12973 | -1 | TGGGAAAAGGTATTGCCTAG(GGG) | 1945 | 70 | 74 |
| 35 | 14409 | -1 | TGGATACACTGAACACACCG(AGG) | 1946 | 85 | 73 |
| 36 | 14896 | -1 | AGTTCTTATATAGCAAACCG(GAG) | 1947 | 88 | 82 |
| 37 | 14921 | 1 | ATAAGAACTGAGCTTTAGAG(AAG) | 1948 | 55 | 84 |
| 38 | 15045 | 1 | GCTGTCTTACTATTTTACTG(CAG) | 1949 | 56 | 83 |
| 39 | 15387 | -1 | GTAATAACTTTGGCACCAGG(CAG) | 1950 | 67 | 91 |
| 40 | 15569 | 1 | ATAAAGTCTTAACTAACAGA(GAG) | 1951 | 52 | 89 |
| 41 | 15850 | -1 | GGAGAACTGCTTGAACCCGG(GAG) | 1952 | 71 | 72 |
| 42 | 17006 | 1 | TAGTTATCATTGGGACACCT(GGG) | 1953 | 82 | 63 |
| 43 | 17865 | 1 | AGTGAGCTGAGATCGCACCA(AGG) | 1954 | 82 | 82 |
| 44 | 18150 | -1 | ATTGGGTCTCCAATACCAAA(CAG) | 1955 | 75 | 75 |
| 45 | 18651 | 1 | GGCCCTGTAGGCGTTACACT(AGG) | 1956 | 92 | 62 |
| 46 | 18855 | 1 | GGTGGGAACAACACACACTG(GGG) | 1957 | 62 | 74 |
| 47 | 19346 | 1 | CCACATGGATGGATACACAA(GGG) | 1958 | 68 | 72 |
| 48 | 20999 | 1 | AGTCAGCTATGATTGCACCA(CAG) | 1959 | 75 | 88 |
| 49 | 21248 | -1 | GCTTGTACGCAAATAACAGG(GAG) | 1960 | 83 | 86 |
| 50 | 23890 | -1 | AGCCCTAAACCCGTAAAATG(GGG) | 1961 | 81 | 63 |
| 51 | 24449 | 1 | GCTCAGCTGAACCAGAGCAA(GAG) | 1962 | 58 | 89 |
| 52 | 24871 | -1 | AATCTGATTTGGCGACACAA(AGG) | 1963 | 83 | 63 |
| 53 | 25247 | -1 | TTGCCCACTGGTGATCACCA(GGG) | 1964 | 69 | 72 |
| 54 | 25468 | -1 | GTATGCATAGGGGTATACTT(TAG) | 1965 | 83 | 78 |
| 55 | 25928 | 1 | TATAGACAAGTCCACGAACC(AGG) | 1966 | 87 | 64 |
| 56 | 25930 | 1 | TAGACAAGTCCACGAACCAG(GAG) | 1967 | 85 | 73 |
| 57 | 26295 | -1 | ATACCTCAGACCGGGCATGG(TGG) | 1968 | 78 | 73 |
| 58 | 26495 | 1 | AATGAAGTGGAAGTACACAG(TAG) | 1969 | 55 | 81 |
| 59 | 26796 | 1 | ATAAGATGGTCACAGCTTGG(GGG) | 1970 | 63 | 74 |
| 60 | 26974 | 1 | TCCTGCCTCAGCCTTCCGAG(GAG) | 1971 | 55 | 84 |
| 61 | 27331 | -1 | GCTTATGGGATTAACCCACA(AGG) | 1972 | 80 | 65 |
| 62 | 27917 | 1 | GGAGCCACAGATTGTTAGCA(GAG) | 1973 | 71 | 75 |
| 63 | 28255 | -1 | ACACGGAAAACAAATCCAGG(AAG) | 1974 | 51 | 87 |
| 64 | 28522 | -1 | TGGGAGATCAACATGCCTAG(TGG) | 1975 | 69 | 73 |
| 65 | 29289 | 1 | GCACCTGCTCCATATTTAGT(AAG) | 1976 | 73 | 75 |
| 66 | 29487 | -1 | CTTGAGCCGTCAAAGACACA(CAG) | 1977 | 67 | 81 |
| 67 | 29862 | -1 | GTATTACCACTTCGTGAAAA(GAG) | 1978 | 85 | 62 |
| 68 | 29864 | 1 | GCTTGTTTACTCTTTTCACG(AAG) | 1979 | 66 | 91 |
| 69 | 29990 | 1 | CCATCTTTGTTGTTTCAAGG(CAG) | 1980 | 58 | 83 |
| 70 | 30043 | 1 | GAATACTCCCAAATTCAGGG(AGG) | 1981 | 67 | 76 |
| 71 | 30500 | 1 | AGAGCATACTGACCTCAACG(TGG) | 1982 | 83 | 77 |
| 72 | 30917 | 1 | TAGATACCCATCTTATACAC(AGG) | 1983 | 82 | 60 |
| 73 | 31112 | 1 | ACACTGCTGCTTCACATCAG(GGG) | 1984 | 61 | 70 |
| 74 | 31620 | -1 | GATACCATTAACTATCACCT(GGG) | 1985 | 73 | 71 |
| 75 | 33443 | 1 | GAGACTCTATTCTAAACGTG(AGG) | 1986 | 85 | 63 |
| 76 | 33616 | 1 | TAACTGCAGTAGTTGACCAT(TGG) | 1987 | 82 | 64 |
| 77 | 34247 | -1 | TTTGCTATGACACAGTACAG(AAG) | 1988 | 66 | 80 |
| 78 | 34544 | 1 | TCTTAGACGGTATAAAGTGG(GAG) | 1989 | 80 | 72 |
| 79 | 35281 | 1 | GAGGGTCACTTGAATCCCAG(AGG) | 1990 | 68 | 74 |
| 80 | 36749 | 1 | TTTTAAATTTGATTTCCGAA(GAG) | 1991 | 56 | 85 |
| 81 | 37237 | -1 | TTCGTGACCTGACAATTGGG(CAG) | 1992 | 83 | 72 |
| 82 | 37670 | 1 | AAGCCCTAAATCAATGCCGA(GGG) | 1993 | 84 | 61 |
| 83 | 37739 | 1 | GGGCATGTCCCTTTCATACA(GAG) | 1994 | 72 | 79 |
| 84 | 37955 | -1 | GCACTCTTCCCAGGATACAA(GAG) | 1995 | 55 | 92 |
| 85 | 38493 | -1 | GGAATATTCCTAGTCCCAAG(AGG) | 1996 | 77 | 72 |
| 86 | 38734 | 1 | CCTCTCATAAGAAATCACTG(GAG) | 1997 | 56 | 86 |
| 87 | 39328 | -1 | TACACTGTAAACGGCCTGAG(AGG) | 1998 | 85 | 66 |
| 88 | 39329 | -1 | TTACACTGTAAACGGCCTGA(GAG) | 1999 | 86 | 70 |
| 89 | 39457 | -1 | CCCCAAAATCGATTAAGCTG(AGG) | 2000 | 84 | 66 |
| 90 | 39551 | -1 | CCTCCCTCATGGGAACATGG(AGG) | 2001 | 62 | 70 |
| 91 | 40096 | -1 | GCACAGTCTGAGCATGTACA(GAG) | 2002 | 66 | 90 |
| 92 | 40431 | 1 | GGTGCTAGAGAACAGCCAAT(CAG) | 2003 | 73 | 92 |
| 93 | 41623 | -1 | CGTCATGTAGAATATGGCAG(AAG) | 2004 | 71 | 83 |
| 94 | 42582 | 1 | AAGACATGTTACATTGTAGG(GGG) | 2005 | 63 | 70 |
| 95 | 42636 | 1 | AGTCAACTCTGCAAAACAAG(GAG) | 2006 | 52 | 83 |
| 96 | 42662 | 1 | TTAATGAAAGCCAATCATCG(AGG) | 2007 | 74 | 74 |
| 97 | 42857 | 1 | ACTCTGGTAACTCCACCTGG(AGG) | 2008 | 66 | 76 |
| 98 | 43791 | -1 | AGCTATAGTAGAATCCTGTG(TGG) | 2009 | 76 | 75 |
| 99 | 43824 | 1 | TGTACATAGACCCAGCACAA(GGG) | 2010 | 71 | 76 |
| 100 | 45140 | 1 | TTAGGAACCAGGCTGCACAG(CAG) | 2011 | 60 | 89 |
| 101 | 45255 | 1 | TATTGTGAACTGCACATACG(AGG) | 2012 | 81 | 65 |
| 102 | 45256 | 1 | ATTGTGAACTGCACATACGA(GGG) | 2013 | 88 | 71 |
| 103 | 45416 | -1 | GATAACACCTGGCAATCCAG(CAG) | 2014 | 73 | 85 |
| 104 | 45427 | -1 | GCTCTCTTAGTGATAACACC(TGG) | 2015 | 81 | 60 |
| 105 | 46134 | 1 | AAACTGTTAACACAAGAGGG(AGG) | 2016 | 65 | 71 |
| 106 | 46321 | 1 | CAACAGACTGTAAGCCCTAG(AGG) | 2017 | 72 | 70 |
| 107 | 46346 | -1 | GGGACACAATACTAACTAGG(TGG) | 2018 | 81 | 72 |
| 108 | 46675 | -1 | GATTTTAAGGTTTACCCCCG(CAG) | 2019 | 87 | 79 |
| 109 | 47862 | -1 | TGACACTCTGGAACATTAGA(GAG) | 2020 | 71 | 73 |
| 110 | 47868 | 1 | TCACTCTCTCTAATGTTCCA(GAG) | 2021 | 59 | 81 |
| 111 | 47960 | -1 | TTAAGAGTATGAAATCCTAC(AAG) | 2022 | 64 | 84 |
| 112 | 48743 | 1 | GGAGGTCGCTTGAGTGCACG(AGG) | 2023 | 88 | 60 |
| 113 | 48893 | -1 | ATATTGCGTTTATACCACAG(AAG) | 2024 | 71 | 74 |
| 114 | 49353 | -1 | TTACCATTGAGAGATCCTTG(GAG) | 2025 | 71 | 72 |
| 115 | 50070 | 1 | AAGGTGATGTTATCGAACAT(AGG) | 2026 | 88 | 63 |
| 116 | 50074 | 1 | TGATGTTATCGAACATAGGA(GAG) | 2027 | 82 | 65 |
| 117 | 50130 | -1 | GACAAGGATACGCTTAACCC(TGG) | 2028 | 92 | 62 |
| 118 | 50147 | 1 | TTAAGCGTATCCTTGTCAGC(CAG) | 2029 | 87 | 69 |
| 119 | 50619 | -1 | GATCCATTAGAAATGCTGAT(CAG) | 2030 | 57 | 88 |
| 120 | 51722 | -1 | GCACTCCAGCCTGAACCAGA(GAG) | 2031 | 67 | 81 |
| 121 | 51778 | -1 | GGAGAATTACTTGAACCCAG(CAG) | 2032 | 54 | 82 |
| 122 | 53493 | -1 | GTTAATAATCATGCTCCGAA(GAG) | 2033 | 87 | 74 |
| 123 | 53495 | -1 | GAGTTAATAATCATGCTCCG(AAG) | 2034 | 82 | 86 |
| 124 | 53560 | 1 | GAGGTTATGTCATCTCCACA(GGG) | 2035 | 66 | 73 |
| 125 | 54056 | 1 | TCTTGCTTATTGCTTGACAA(CAG) | 2036 | 64 | 81 |
| 126 | 54511 | -1 | GAGCCATGATCACACCACTG(CGG) | 2037 | 69 | 84 |
| 127 | 54692 | -1 | AAGGCGGGTGAATCACTTGA(GGG) | 2038 | 81 | 65 |
| 128 | 54693 | -1 | CAAGGCGGGTGAATCACTTG(AGG) | 2039 | 84 | 62 |
| 129 | 55118 | 1 | ACCCTGGTCAATAGCCACAG(TGG) | 2040 | 70 | 72 |
| 130 | 55121 | -1 | AATGCAGGTATACTCCACTG(TGG) | 2041 | 79 | 79 |
| 131 | 55162 | 1 | ATACACTCTTGACAACCATA(GAG) | 2042 | 74 | 78 |
| 132 | 56004 | 1 | CCCCATTCTCAAATTCCAAG(CAG) | 2043 | 57 | 87 |
| 133 | 56439 | -1 | GATGTGTTCTTCAAGTAGCA(GAG) | 2044 | 66 | 86 |
| 134 | 57073 | 1 | GAGACTAATGTCGAACAACA(TGG) | 2045 | 81 | 67 |
| 135 | 57802 | -1 | TTTTAGCTAGAACCAGGGTG(GAG) | 2046 | 71 | 79 |
| 136 | 60122 | 1 | CTGACTAATGAAAACTCCTG(TGG) | 2047 | 62 | 70 |
| 137 | 60257 | -1 | TAGGCTGACAGGGTTACAGA(GGG) | 2048 | 66 | 70 |
| 138 | 60516 | -1 | ATGCTAGTGGTACCATGCAT(GGG) | 2049 | 81 | 61 |
| 139 | 60808 | -1 | GCCTCACTAGACTTTCAGTG(TAG) | 2050 | 76 | 79 |
| 140 | 61932 | -1 | GCACCCCAAAACAATTACCA(CAG) | 2051 | 66 | 85 |
| 141 | 62404 | 1 | TTAAGCCAAAGCCTAATCCA(CAG) | 2052 | 74 | 71 |
| 142 | 62737 | -1 | GCTGCATTATCCCCTAACAA(GAG) | 2053 | 81 | 94 |
| 143 | 63063 | -1 | CTATTTATAGAGCACAGGCA(GAG) | 2054 | 56 | 85 |
| 144 | 63147 | -1 | TGAGCTATAAGTATCAACAC(TGG) | 2055 | 80 | 63 |
| 145 | 63203 | -1 | CCAGGCACATTGTCAATAGG(CAG) | 2056 | 79 | 75 |
| 146 | 63236 | 1 | GTCACTCAAGAGCTCTAACG(GAG) | 2057 | 87 | 68 |
| 147 | 63239 | 1 | ACTCAAGAGCTCTAACGGAG(AGG) | 2058 | 89 | 68 |
| 148 | 63307 | 1 | TCTGTAGCCTATGGGCCAAA(GAG) | 2059 | 71 | 72 |
| 149 | 66208 | 1 | TTAGGATTGACTTGGCGATG(CGG) | 2060 | 84 | 61 |
| 150 | 66433 | 1 | GTTTGTAGTTCTCCTCGAAG(AGG) | 2061 | 84 | 62 |
| 151 | 66811 | -1 | AGGGAGACTTTATAAACCGG(AGG) | 2062 | 85 | 73 |
| 152 | 67111 | -1 | AGTGAGCCAAAATCGCGCCA(CGG) | 2063 | 95 | 69 |
| 153 | 67288 | -1 | AGGTGGGCAGAACACAACGT(CAG) | 2064 | 79 | 74 |
| 154 | 67480 | 1 | AAACTTACAATCATGGTCGA(AGG) | 2065 | 89 | 61 |
| 155 | 67959 | -1 | GGCTTTTTATTTGTATGCGG(CAG) | 2066 | 79 | 72 |
| 156 | 67997 | -1 | CATTTCTCACCGTATTCAGG(AGG) | 2067 | 82 | 62 |
| 157 | 67998 | -1 | GCATTTCTCACCGTATTCAG(GAG) | 2068 | 83 | 65 |
| 158 | 68003 | 1 | ACTTACCTCCTGAATACGGT(GAG) | 2069 | 90 | 67 |
| 159 | 68307 | -1 | GATTCCTCACTTACTAACCA(CGG) | 2070 | 75 | 73 |
| 160 | 68314 | 1 | ATTACCGTGGTTAGTAAGTG(AGG) | 2071 | 88 | 60 |
| 161 | 68419 | -1 | CCTAAGTTGAAGGAACGTCA(GAG) | 2072 | 83 | 63 |
| 162 | 68756 | -1 | CGCATCTATCAATGTCACCT(TGG) | 2073 | 80 | 62 |
| 163 | 69363 | -1 | GGAACAAAAGAGACGACAGT(GGG) | 2074 | 69 | 72 |
| 164 | 69460 | 1 | TATGACCATGAATAACCCAC(CGG) | 2075 | 68 | 71 |
| 165 | 69464 | -1 | CATTTTTGGTCCAGTCCGGT(GGG) | 2076 | 92 | 61 |
| 166 | 69955 | 1 | AGGGTGTGTCCATAACCCAA(CAG) | 2077 | 80 | 69 |
| 167 | 70069 | -1 | GTAAAAGTGTACCTAAACAC(AAG) | 2078 | 73 | 74 |
| 168 | 70165 | 1 | TCACTTTTCTTCGTGACCCG(TGG) | 2079 | 86 | 70 |
| 169 | 70656 | -1 | TTAATCTTAGGCTTAGTAAG(CAG) | 2080 | 69 | 83 |
| 170 | 70888 | 1 | GAGCCACCTAGACAATACAG(AAG) | 2081 | 74 | 74 |
| 171 | 70890 | 1 | GCCACCTAGACAATACAGAA(GGG) | 2082 | 69 | 75 |
| 172 | 71166 | 1 | TCACCACTACCTAATTAGAG(AAG) | 2083 | 80 | 79 |
| 173 | 71983 | 1 | GAGAGTGGGGTTAAACACCA(GGG) | 2084 | 73 | 76 |
| 174 | 72722 | 1 | TGTAGGGGCTTTGATCCAGT(CAG) | 2085 | 79 | 74 |
| 175 | 73080 | -1 | CTGCTAGATAGCTTAGAACC(AGG) | 2086 | 83 | 61 |
| 176 | 73091 | 1 | CCTGGTTCTAAGCTATCTAG(CAG) | 2087 | 82 | 62 |
| 177 | 73609 | 1 | TCATCTGTATGCACTCTCAG(AAG) | 2088 | 64 | 81 |
| 178 | 74688 | -1 | GCTGTCTCAGCCAATCACAG(CAG) | 2089 | 63 | 82 |
| 179 | 75558 | -1 | GCTTAAATGCCATCACCTCA(GAG) | 2090 | 64 | 83 |
| 180 | 76917 | -1 | TCTTTCCTCCTGTTTCGGTG(GAG) | 2091 | 74 | 72 |
| 181 | 77252 | 1 | GCCAGATTATGTGTAGACTG(TGG) | 2092 | 76 | 74 |
| 182 | 77679 | -1 | GTTTTAGCCCAATATAACCA(CAG) | 2093 | 71 | 87 |
| 183 | 77846 | -1 | GCACATGATACTCTACACTC(TGG) | 2094 | 81 | 62 |
| 184 | 77927 | -1 | GAAGAGTATATCCCCAACGA(AGG) | 2095 | 89 | 66 |
| 185 | 78181 | -1 | GGAATGCAGCATAACGGCAA(AGG) | 2096 | 90 | 61 |
| 186 | 78444 | -1 | CCATGTATATTAAATCTACA(GAG) | 2097 | 56 | 80 |
| 187 | 79522 | -1 | AACACTCCTGAACCTCGGGA(AGG) | 2098 | 85 | 61 |
| 188 | 80328 | 1 | TACTGAGTCTGCCTCTTCCG(GAG) | 2099 | 75 | 74 |
| 189 | 80331 | -1 | TATGTGGTACAATACTCCGG(AAG) | 2100 | 96 | 74 |
| 190 | 80539 | 1 | TAGGCTATACCACATAGCCT(AGG) | 2101 | 81 | 63 |
| 191 | 80545 | -1 | GTATAGGCTACCATACACCT(AGG) | 2102 | 88 | 69 |
| 192 | 80585 | 1 | GGTTTGTATAAGTGCACCCT(AGG) | 2103 | 82 | 60 |
| 193 | 80970 | -1 | GAAAATAAATTAAGGCAACG(TGG) | 2104 | 66 | 72 |
| 194 | 81615 | 1 | GTGCTCGAATTAACACAAGA(CAG) | 2105 | 83 | 66 |
| 195 | 81715 | 1 | TCTTCTTTCTGGAAAACGAA(GAG) | 2106 | 51 | 88 |
| 196 | 82630 | 1 | CGTGTGTAGTCAGTGTCCAG(AGG) | 2107 | 74 | 70 |
| 197 | 82993 | -1 | TGATATACTCAGGAAGGCGA(GAG) | 2108 | 74 | 72 |
| 198 | 83017 | -1 | GTACTTAACTAGGACCCCAT(GGG) | 2109 | 87 | 69 |
| 199 | 84026 | -1 | CCAAACCATGAAAACCCTAG(AAG) | 2110 | 71 | 74 |
| 200 | 84279 | -1 | GCATGGTAGTGGTACCCAAA(CAG) | 2111 | 81 | 79 |
| 201 | 85965 | 1 | GACAACTACCTAATGCATGC(AGG) | 2112 | 84 | 61 |
| 202 | 86012 | 1 | ATAGGTGGAGCAAACCACCA(TAG) | 2113 | 72 | 75 |
| 203 | 86606 | 1 | GGTGGGCAATGAGAACACAT(GGG) | 2114 | 61 | 74 |
| 204 | 86633 | 1 | GAGAAGATCATCACACACTG(GGG) | 2115 | 65 | 74 |
| 205 | 86724 | 1 | ATGGGTGCAGCAAACCACCA(TGG) | 2116 | 64 | 73 |
| 206 | 86730 | -1 | ACATGGGTAGACACGTGCCA(TGG) | 2117 | 83 | 75 |
| 207 | 87379 | -1 | TACGACAAAGAAGATCATGT(AGG) | 2118 | 67 | 72 |
| 208 | 87621 | 1 | CGGTTACAACAGAGGCTCTG(CGG) | 2119 | 63 | 71 |
| 209 | 87627 | -1 | AGAAGGCCAAGCATATACCG(CAG) | 2120 | 85 | 94 |
| 210 | 88070 | 1 | ACACACAATGGATTTCCCCA(GAG) | 2121 | 58 | 88 |
| 211 | 88144 | -1 | ACTGTTTTAGTCATACCCCA(TAG) | 2122 | 71 | 89 |
| 212 | 88422 | 1 | GGAGTCTAATGTATTAGGGG(AGG) | 2123 | 83 | 64 |
| 213 | 88874 | 1 | ACTACCTAGGGAATTCCCAG(AAG) | 2124 | 68 | 83 |
| 214 | 89604 | -1 | TATGCCCTTCGACACCAAGG(TGG) | 2125 | 78 | 72 |
| 215 | 89611 | 1 | GTTTCCACCTTGGTGTCGAA(GGG) | 2126 | 86 | 64 |
| 216 | 89689 | 1 | TGCTAAATGTGTATCACCCG(AGG) | 2127 | 88 | 68 |
| 217 | 89738 | 1 | TTTAGGGTAAGAGAACTCGG(GAG) | 2128 | 83 | 74 |
| 218 | 90216 | -1 | TTATAAGCAGGGAGGCCTGA(GAG) | 2129 | 54 | 81 |
| 219 | 90645 | -1 | GAAGTTGCCCAATACCAAAG(AGG) | 2130 | 70 | 72 |
| 220 | 90646 | -1 | AGAAGTTGCCCAATACCAAA(GAG) | 2131 | 64 | 82 |
| 221 | 91214 | 1 | TTTTCTGCAAGGCGAAGCAG(CAG) | 2132 | 72 | 76 |
| 222 | 91444 | 1 | TGCTGTGGACTGCAACGGTG(TGG) | 2133 | 82 | 68 |
| 223 | 91457 | -1 | CTGAGCGTCCATCAACCAGG(GAG) | 2134 | 81 | 69 |
| 224 | 92139 | 1 | GTAGCTCCTAAGTTGAAACG(GAG) | 2135 | 90 | 85 |
| 225 | 92140 | 1 | TAGCTCCTAAGTTGAAACGG(AGG) | 2136 | 87 | 72 |
| 226 | 92408 | 1 | AAGGTCTACGAGTCACTAAG(TGG) | 2137 | 88 | 64 |
| 227 | 93703 | 1 | GTAAGAGACAACCATTACAG(GAG) | 2138 | 67 | 81 |
| 228 | 94028 | -1 | ACTGGCTGTATATCATAGGA(GAG) | 2139 | 79 | 74 |
| 229 | 94038 | 1 | GCTCTCCTATGATATACAGC(CAG) | 2140 | 83 | 68 |
| 230 | 94204 | 1 | GCACGACCAATCAAATACAC(AAG) | 2141 | 87 | 70 |
| 231 | 95047 | 1 | TGTCATGGGACTAAAAACAC(AGG) | 2142 | 60 | 71 |
| 232 | 95431 | -1 | GCAAATCTGTACCACCAAGG(TGG) | 2143 | 73 | 73 |
| 233 | 95434 | -1 | TGTGCAAATCTGTACCACCA(AGG) | 2144 | 72 | 77 |
| 234 | 95600 | 1 | GGAACACCACCCAATGACTG(AGG) | 2145 | 74 | 74 |
| 235 | 95871 | 1 | ATAAAAGGTTACCATCTTGG(GAG) | 2146 | 61 | 82 |
| 236 | 96250 | -1 | CTATATGCCAGGCTAATAAG(CAG) | 2147 | 71 | 81 |
| 237 | 96762 | -1 | TGCTAACTCAGCGAGCACAT(GGG) | 2148 | 82 | 61 |
| 238 | 97850 | 1 | AGTTCTGCGATCATTCAGAC(TGG) | 2149 | 81 | 60 |
| 239 | 98726 | -1 | GGTTACCTAGAGCCCCTACT(GAG) | 2150 | 83 | 66 |
| 240 | 98747 | -1 | TGATGGCCAACACTAAGGTG(AGG) | 2151 | 69 | 73 |
| 241 | 99300 | -1 | TTACTAGTATAGCTTCAAGA(GAG) | 2152 | 64 | 88 |
| 242 | 99408 | -1 | ATAGAGGCTAGTCTTACACA(TGG) | 2153 | 72 | 72 |
| 243 | 99426 | 1 | TAAGACTAGCCTCTATAGCA(AGG) | 2154 | 80 | 63 |
| 244 | 99753 | -1 | CACGCGATGCTATAGGCCAG(TGG) | 2155 | 87 | 63 |
| 245 | 99765 | 1 | CACTGGCCTATAGCATCGCG(TGG) | 2156 | 97 | 66 |
| 246 | 99940 | -1 | ACTGTGACAAGTCAACGTGG(CAG) | 2157 | 81 | 77 |
| 247 | 99943 | -1 | CCAACTGTGACAAGTCAACG(TGG) | 2158 | 86 | 75 |
| 248 | 101373 | -1 | GTAGTTCCTCCATTAGTCAA(GAG) | 2159 | 74 | 92 |
| 249 | 101609 | 1 | TCTATATCAGGAAACTTGCG(AAG) | 2160 | 82 | 65 |
| 250 | 102970 | -1 | ACACGGATAAGACCACATGA(GAG) | 2161 | 72 | 74 |
| 251 | 103743 | -1 | TTACCAGATGAATCTTCAGG(AAG) | 2162 | 60 | 88 |
| 252 | 104091 | 1 | TGATGTATCCATGATCCGCA(AAG) | 2163 | 92 | 85 |
| 253 | 105545 | -1 | TACTCGCCCATAGATATCGA(GGG) | 2164 | 96 | 70 |
| 254 | 105546 | -1 | CTACTCGCCCATAGATATCG(AGG) | 2165 | 97 | 62 |
| 255 | 108599 | 1 | CTCAACTGGAAATCGTCCCA(GGG) | 2166 | 85 | 64 |
| 256 | 108829 | 1 | AGCTAACATGATACTAACCA(GGG) | 2167 | 76 | 79 |
| 257 | 109542 | 1 | ATAAAGCTATAGTAACCAAA(CAG) | 2168 | 58 | 93 |
| 258 | 110187 | 1 | CTCAACATCACTAATCACCA(GGG) | 2169 | 69 | 72 |
| 259 | 110646 | 1 | GGCAACATGAATGAACCTGG(AGG) | 2170 | 65 | 78 |
| 260 | 110720 | -1 | CTACTTCTATGACAACCCTT(TAG) | 2171 | 71 | 72 |
| 261 | 110756 | 1 | AGTAGAATAGTGGTTATCGG(AGG) | 2172 | 87 | 66 |
| 262 | 110856 | -1 | CTAGTCATCCAACGATTCAA(TAG) | 2173 | 91 | 73 |
| 263 | 112186 | 1 | TCTCTGGCCCGGTACTCACG(TAG) | 2174 | 92 | 61 |
| 264 | 112189 | -1 | ACACAGGGAGAAAACTACGT(GAG) | 2175 | 72 | 71 |
| 265 | 112238 | -1 | GTCCAAATCCAATATAACTG(GGG) | 2176 | 68 | 74 |
| 266 | 112970 | 1 | TGCCACGATAAGGCCCAAAG(AGG) | 2177 | 80 | 68 |
| 267 | 113933 | -1 | AATTTCATCAACAAGCCAGG(GAG) | 2178 | 56 | 83 |
| 268 | 114417 | 1 | CCACAGATCAGCAGTCCACG(TGG) | 2179 | 77 | 73 |
| 269 | 114563 | 1 | GTAGAGAAAGAAATAGAACG(AAG) | 2180 | 51 | 94 |
| 270 | 114615 | 1 | GGAAGGGCAAAACTTTCCCA(GAG) | 2181 | 52 | 86 |
| 271 | 119671 | 1 | AAGATTGTAGAGACCTCAAG(GGG) | 2182 | 60 | 76 |
| 272 | 124929 | -1 | ACAGATGGTGATGACCAATG(GGG) | 2183 | 72 | 77 |
| 273 | 126886 | 1 | GGGGCCGTGCAAATATATGG(AGG) | 2184 | 90 | 63 |
| 274 | 126887 | 1 | GGGCCGTGCAAATATATGGA(GGG) | 2185 | 82 | 63 |
| 275 | 127078 | -1 | CCTCAAGTGATCGCCCACCT(CGG) | 2186 | 87 | 60 |
| 276 | 127540 | 1 | GGGAGCTTAGACTAGTATGG(TAG) | 2187 | 89 | 68 |
| 277 | 131325 | 1 | AGAGTTGCACAGTAGCCCAA(TAG) | 2188 | 70 | 82 |
| 278 | 131352 | -1 | TCTGGATTATTCTCTCTGGA(CAG) | 2189 | 55 | 83 |
| 279 | 131411 | 1 | AGAGATGCACAATAGCCCGA(CAG) | 2190 | 86 | 91 |
| 280 | 131432 | -1 | CTATTCCGTTTGAATAGCAG(AAG) | 2191 | 72 | 85 |
| 281 | 131938 | -1 | AGGATCCCAGGACTACCAGG(TGG) | 2192 | 68 | 74 |
| 282 | 132160 | -1 | GATGTCTCCACGGTACATGG(AGG) | 2193 | 84 | 72 |
| 283 | 132164 | 1 | AACTTATCCTCCATGTACCG(TGG) | 2194 | 85 | 66 |
| 284 | 132166 | 1 | CTTATCCTCCATGTACCGTG(GAG) | 2195 | 88 | 68 |
| 285 | 132402 | -1 | AGTTTGGGTGGAATTCCAGG(CAG) | 2196 | 51 | 91 |
| 286 | 132545 | -1 | ACTAAAGTGACAGATAGTCA(GGG) | 2197 | 64 | 70 |
| 287 | 135192 | 1 | TCACTGCAAACACAACCCTG(AGG) | 2198 | 65 | 77 |
| 288 | 135662 | -1 | TAGGTAAACACGTGTCATGG(GGG) | 2199 | 77 | 71 |
| 289 | 135663 | -1 | ATAGGTAAACACGTGTCATG(GGG) | 2200 | 83 | 64 |
| 290 | 138647 | 1 | GTTTACATATTATTTTAACG(AAG) | 2201 | 54 | 90 |
| 291 | 140378 | 1 | GGTCTCAAAACTGAAAACGT(TGG) | 2202 | 68 | 71 |
| 292 | 140459 | -1 | ATTTGCCACATCCAACCCCA(GAG) | 2203 | 51 | 87 |
| 293 | 140907 | 1 | TTAAAGTTCTGGAAGCTGGG(AAG) | 2204 | 52 | 86 |
| 294 | 141052 | -1 | GAGTGAGTTAGATATCACAA(GAG) | 2205 | 71 | 75 |
| 295 | 144206 | 1 | AAAAAGACGGACGGATCATG(AGG) | 2206 | 85 | 62 |
| 296 | 144318 | 1 | TGTAGTCTCAGCTACTAGGG(AGG) | 2207 | 60 | 74 |
| 297 | 145243 | 1 | GTACAGTGGTACATAGACCC(CGG) | 2208 | 90 | 72 |
| 298 | 145249 | -1 | TACTCACTCTTTGGGGACCG(GGG) | 2209 | 84 | 64 |
| 299 | 145337 | 1 | CAGTAGGGAGTGGCTATCCG(GGG) | 2210 | 87 | 66 |
| 300 | 145363 | 1 | TCTGGGAAGACATCACAAGG(AGG) | 2211 | 60 | 76 |
| 301 | 145431 | 1 | AGGCAGATAGGCATTCAAGG(CAG) | 2212 | 56 | 85 |
| 302 | 145518 | -1 | CCCTACTATGTTTATCACGT(AGG) | 2213 | 92 | 65 |
| 303 | 145524 | 1 | GAGTGCCTACGTGATAAACA(TAG) | 2214 | 84 | 75 |
| 304 | 145883 | 1 | AGAGTTGAATAGTTGCAACG(GAG) | 2215 | 80 | 64 |
| 305 | 148450 | -1 | ACCACTAGGCTACTATCAGG(TGG) | 2216 | 88 | 63 |
| 306 | 148460 | 1 | TCCACCTGATAGTAGCCTAG(TGG) | 2217 | 88 | 74 |
| 307 | 148475 | 1 | CCTAGTGGTTGTGAGTACAG(CAG) | 2218 | 76 | 81 |
| 308 | 149832 | -1 | TCAGGAACCATATCTTACGT(TGG) | 2219 | 87 | 66 |
| 309 | 150045 | -1 | TCTAACCTCCAAAAGTGTGA(GAG) | 2220 | 67 | 82 |
| 310 | 150239 | -1 | ACTGGCATCCTTACTAGTAG(AGG) | 2221 | 84 | 61 |
| 311 | 152488 | 1 | CCATTTTAGTTACTTCACCG(AAG) | 2222 | 83 | 86 |
| 312 | 153443 | -1 | GGAGAATCACTTGAACCAGG(GAG) | 2223 | 54 | 88 |
| 313 | 154133 | -1 | GGGGTGCCAAGAATACACAA(TAG) | 2224 | 75 | 76 |
| 314 | 154336 | 1 | TTATAATCCCAGCAACTCGG(GAG) | 2225 | 80 | 68 |
| 315 | 154500 | 1 | GGAGGATCACTGAAGCCCAA(GAG) | 2226 | 59 | 83 |
| 316 | 154992 | 1 | ATTGTTAGTGTATAGAAACG(CAG) | 2227 | 76 | 76 |
| 317 | 155184 | -1 | ACAGCACTAGAAGTCCTAGC(CAG) | 2228 | 81 | 68 |
| 318 | 155293 | 1 | ATTGAATACGATGTTAGCTG(TGG) | 2229 | 80 | 60 |
| 319 | 155374 | 1 | GCTACTATAACAGAATACCA(CAG) | 2230 | 66 | 91 |
| 320 | 155448 | 1 | ATGGAAAGTTCATGACTGAG(GGG) | 2231 | 62 | 70 |
| 321 | 155658 | -1 | GCAATTGTGACAGAATTGGG(AGG) | 2232 | 63 | 74 |
| 322 | 155913 | -1 | CATGTACTACGACCAAGTGG(GAG) | 2233 | 84 | 81 |
| 323 | 155915 | -1 | ATCATGTACTACGACCAAGT(GGG) | 2234 | 92 | 69 |
| 324 | 155916 | -1 | GATCATGTACTACGACCAAG(TGG) | 2235 | 92 | 63 |
| 325 | 155918 | 1 | GGACAACTCCCACTTGGTCG(TAG) | 2236 | 88 | 62 |
| 326 | 156439 | -1 | AAGGGTAACAACACACACTG(GGG) | 2237 | 67 | 74 |
| 327 | 157217 | -1 | CTATCAACAGAGTAAGCAGA(CAG) | 2238 | 54 | 81 |
| 328 | 157656 | -1 | ATGGGTGCCGCACACCAACA(TGG) | 2239 | 82 | 65 |
| 329 | 159943 | 1 | GCATCCCTCAGATAAATCCC(AAG) | 2240 | 71 | 82 |
| 330 | 160831 | -1 | TTGAACAACTCATAATTACG(TGG) | 2241 | 83 | 70 |
| 331 | 161670 | 1 | GTAGCCTAATGGTTTCCATG(GGG) | 2242 | 68 | 77 |
| 332 | 161744 | -1 | CAAGTACAAAAAACGATGGG(GGG) | 2243 | 85 | 66 |
| 333 | 162012 | 1 | ATAGTTTCTCAACCCTTGGG(AAG) | 2244 | 70 | 77 |
| 334 | 162135 | -1 | GAAAAACTCATGCACACCAG(AGG) | 2245 | 63 | 71 |
| 335 | 163457 | -1 | TGCAGCAACACCATAACAGT(AGG) | 2246 | 78 | 70 |
| 336 | 163882 | -1 | GTAACCAAAAGAGAGCAATG(GGG) | 2247 | 53 | 82 |
| 337 | 164242 | -1 | ATTGCCTCATGATAACCACA(AGG) | 2248 | 64 | 72 |
| 338 | 164558 | -1 | CATGGTAAAGAGCAACACAA(GAG) | 2249 | 53 | 83 |
| 339 | 164789 | 1 | GCTCTTTTAAAGTTTCCACT(GAG) | 2250 | 55 | 84 |
| 340 | 166597 | -1 | TGAAGTTTAGCAAAGTACCA(GAG) | 2251 | 61 | 80 |
| 341 | 166686 | -1 | AAAGGGATAAAAGAAATCCG(CAG) | 2252 | 56 | 83 |
| 342 | 168072 | -1 | GCATGCCAAGAACTTGACAG(AAG) | 2253 | 62 | 95 |
| 343 | 169562 | 1 | CTTATGAGTGAGAACATGCA(GAG) | 2254 | 57 | 80 |
| 344 | 170590 | -1 | CCTCACAAAAACAAGCAACG(GGG) | 2255 | 72 | 73 |
| 345 | 175111 | -1 | TCAAGTGATTTCGCCCACCT(CGG) | 2256 | 85 | 64 |
| 346 | 180157 | -1 | GTAGGAACACTTGAAGCCAG(GAG) | 2257 | 61 | 84 |

*Searching was based on WT Cas9 PAMs, 347 solutions out of ~34,500 possible guide-RNA sequences are shown.

TABLE 11
Top-Scoring Guide-RNA Target Sites and PAM Sequences in SCN9A for
Programmed Cas9-Nickase Pairs

| | | | | SEQ | | |
|---|---|---|---|---|---|---|
| | | | | ID | Specificity | Efficiency |
| Pair | Position | Strand | Guide-RNA target site (PAM) | NOs | score⁵⁰ | score²⁸ |
| 1 | 6112 | 1 | CAGGAGTTCTAGATCAGCCT(GGG) | 2258 | 66 | 60 |
| | 6050 | −1 | CCAAAGTGATGGGATTGCAG(GGG) | 2259 | 62 | 70 |
| 2 | 14921 | 1 | ATAAGAACTGAGCTTTAGAG(AAG) | 2260 | 55 | 84 |
| | 14896 | −1 | AGTTCTTATATAGCAAACCG(GAG) | 2261 | 88 | 82 |
| 3 | 24369 | 1 | AAGACTGATGAATCCAGCCA(GGG) | 2262 | 61 | 68 |
| | 24306 | −1 | ATCCAAGTCATTAGTCTTGG(GGG) | 2263 | 74 | 64 |
| 4 | 26796 | 1 | ATAAGATGGTCACAGCTTGG(GGG) | 2264 | 63 | 74 |
| | 26751 | −1 | CTCCCATATTTAGCCCAATG(GGG) | 2265 | 76 | 68 |
| 5 | 26974 | 1 | TCCTGCCTCAGCCTTCCGAG(GAG) | 2266 | 55 | 84 |
| | 26943 | −1 | GGAGAATCGCTTGAACCCAG(GAG) | 2267 | 59 | 75 |
| 6 | 27276 | 1 | TTGAGTCCTAGATAGGTGGG(TGG) | 2268 | 71 | 61 |
| | 27249 | −1 | GACTCAAGATCCTAGATAGG(TGG) | 2269 | 79 | 61 |
| 7 | 27277 | 1 | TGAGTCCTAGATAGGTGGGT(GGG) | 2270 | 74 | 64 |
| | 27249 | −1 | GACTCAAGATCCTAGATAGG(TGG) | 2271 | 79 | 61 |
| 8 | 27313 | 1 | AAATTGGGCTGGGACCACTT(AGG) | 2272 | 78 | 60 |
| | 27249 | −1 | GACTCAAGATCCTAGATAGG(TGG) | 2273 | 79 | 61 |
| 9 | 28567 | 1 | TGCATGAAAACCTATCCCCT(GGG) | 2274 | 77 | 66 |
| | 28522 | −1 | TGGGAGATCAACATGCCTAG(TGG) | 2275 | 69 | 73 |
| 10 | 29897 | 1 | AATTACCTCCCACAGCAGCA(CAG) | 2276 | 50 | 76 |
| | 29862 | −1 | GTATTACCACTTCGTGAAAA(GAG) | 2277 | 85 | 62 |
| 11 | 29916 | 1 | ACAGGTTACTCAAAAGCCCA(GAG) | 2278 | 60 | 62 |
| | 29862 | −1 | GTATTACCACTTCGTGAAAA(GAG) | 2279 | 85 | 62 |
| 12 | 29919 | 1 | GGTTACTCAAAAGCCCAGAG(GAG) | 2280 | 58 | 73 |
| | 29862 | −1 | GTATTACCACTTCGTGAAAA(GAG) | 2281 | 85 | 62 |
| 13 | 30917 | 1 | TAGATACCCATCTTATACAC(AGG) | 2282 | 82 | 60 |
| | 30869 | −1 | GTCTTTAACTATCATCCATG(TGG) | 2283 | 67 | 67 |
| 14 | 30917 | 1 | TAGATACCCATCTTATACAC(AGG) | 2284 | 82 | 60 |
| | 30864 | −1 | TAACTATCATCCATGTGGAA(GGG) | 2285 | 62 | 60 |
| 15 | 31268 | 1 | TTTGCACAAAGGATTGTAGG(TGG) | 2286 | 63 | 64 |
| | 31215 | −1 | CAAGATACCAAACTAAGAGG(TGG) | 2287 | 62 | 63 |
| 16 | 32326 | 1 | TAGCTGAGATCCACTCCCCT(CGG) | 2288 | 72 | 69 |
| | 32274 | −1 | GATGTTTGCTTGAGCCCCTG(GGG) | 2289 | 66 | 65 |
| 17 | 32400 | 1 | CTTTTTCAATGAGGAAACCG(TGG) | 2290 | 64 | 62 |
| | 32338 | −1 | TATAATCCCAGCACTTTGGG(AGG) | 2291 | 8 | 64 |
| 18 | 36029 | 1 | AATAGGAGACATAGTTCCTG(AGG) | 2292 | 61 | 68 |
| | 35994 | −1 | CACTGGTGAGGAAGTTACAC(GGG) | 2293 | 79 | 63 |
| 19 | 36029 | 1 | AATAGGAGACATAGTTCCTG(AGG) | 2294 | 61 | 68 |
| | 35976 | −1 | ACGGGTAGTCTGTTAGAAAG(AGG) | 2295 | 67 | 60 |
| 20 | 39372 | 1 | TTAGAGCCAAAGGAGCAAGT(AAG) | 2296 | 58 | 62 |
| | 39329 | −1 | TTACACTGTAAACGGCCTGA(GAG) | 2297 | 86 | 70 |
| 21 | 39469 | 1 | CTCAGCTTAATCGATTTTGG(GGG) | 2298 | 77 | 60 |
| | 39418 | −1 | TAGACCACAATTTCACCTGG(AGG) | 2299 | 66 | 64 |
| 22 | 39847 | 1 | TAAGGGAACACCAAAAGCAC(AGG) | 2300 | 62 | 61 |
| | 39815 | −1 | TAGCCAAACCTGCTAGAAAG(AGG) | 2301 | 61 | 64 |
| 23 | 40326 | 1 | CTATGCTTCTGAAAGTTAGC(AAG) | 2302 | 58 | 61 |
| | 40298 | −1 | GCATAGTTACTGGAGTGAGG(CAG) | 2303 | 61 | 75 |
| 24 | 40326 | 1 | CTATGCTTCTGAAAGTTAGC(AAG) | 2304 | 58 | 61 |
| | 40298 | −1 | GCATAGTTACTGGAGTGAGG(CAG) | 2305 | 61 | 75 |
| 25 | 43824 | 1 | TGTACATAGACCCAGCACAA(GGG) | 2306 | 71 | 76 |
| | 43791 | −1 | AGCTATAGTAGAATCCTGTG(TGG) | 2307 | 76 | 75 |
| 26 | 44028 | 1 | TCCATCACCACTACACACAA(TGG) | 2308 | 60 | 60 |
| | 43991 | −1 | CAGAGCCTTGACACCTGCCG(TGG) | 2309 | 70 | 68 |
| 27 | 45061 | 1 | GATCTGTTTATAGGCCACAG(TGG) | 2310 | 61 | 66 |
| | 45014 | −1 | TCTGTGGCTTATACAACTGG(GGG) | 2311 | 75 | 70 |
| 28 | 45061 | 1 | GATCTGTTTATAGGCCACAG(TGG) | 2312 | 61 | 66 |
| | 45015 | −1 | ATCTGTGGCTTATACAACTG(GGG) | 2313 | 65 | 64 |
| 29 | 45113 | 1 | CAGTGACAGATACTGGTCCA(TGG) | 2314 | 66 | 60 |
| | 45064 | −1 | ACCCCTGATCTAACCCACTG(TGG) | 2315 | 73 | 68 |
| 30 | 45255 | 1 | TATTGTGAACTGCACATACG(AGG) | 2316 | 81 | 65 |
| | 45194 | −1 | GCCAAGGCTGATCTAACAGG(AGG) | 2317 | 75 | 69 |
| 31 | 45255 | 1 | TATTGTGAACTGCACATACG(AGG) | 2318 | 81 | 65 |
| | 45210 | −1 | TCTCTGAGAGTCTAATGCCA(AGG) | 2319 | 62 | 64 |
| 32 | 45256 | 1 | ATTGTGAACTGCACATACGA(GGG) | 2320 | 88 | 71 |
| | 45194 | −1 | GCCAAGGCTGATCTAACAGG(AGG) | 2321 | 75 | 69 |
| 33 | 45256 | 1 | ATTGTGAACTGCACATACGA(GGG) | 2322 | 88 | 71 |
| | 45210 | −1 | TCTCTGAGAGTCTAATGCCA(AGG) | 2323 | 62 | 64 |
| 34 | 50918 | 1 | AATGGTGCAGATAGTAAGGA(CAG) | 2324 | 65 | 60 |
| | 50879 | −1 | ATAAATCTATTCCAAGACAA(AAG) | 2325 | 51 | 73 |
| 35 | 53558 | 1 | GAGAGGTTATGTCATCTCCA(CAG) | 2326 | 64 | 69 |
| | 53495 | −1 | GAGTTAATAATCATGCTCCG(AAG) | 2327 | 82 | 86 |
| 36 | 57832 | 1 | AAAATGGACATGGATACCCT(AGG) | 2328 | 70 | 65 |
| | 57804 | −1 | CATTTTAGCTAGAACCAGGG(TGG) | 2329 | 71 | 68 |
| 37 | 57832 | 1 | AAAATGGACATGGATACCCT(AGG) | 2330 | 70 | 65 |
| | 57807 | −1 | GTCCATTTTAGCTAGAACCA(GGG) | 2331 | 71 | 65 |
| 38 | 63236 | 1 | GTCACTCAAGAGCTCTAACG(GAG) | 2332 | 87 | 68 |
| | 63203 | −1 | CCAGGCACATTGTCAATAGG(CAG) | 2333 | 79 | 75 |
| 39 | 63246 | 1 | AGCTCTAACGGAGAGGTACA(AGG) | 2334 | 78 | 62 |
| | 63221 | −1 | GTTAGAGCTCTTGAGTGACC(AGG) | 2335 | 75 | 61 |
| 40 | 63247 | 1 | GCTCTAACGGAGAGGTACAA(GGG) | 2336 | 78 | 64 |
| | 63221 | −1 | GTTAGAGCTCTTGAGTGACC(AGG) | 2337 | 75 | 61 |
| 41 | 66136 | 1 | TTTGGGTTACTGTAGCCTTG(TAG) | 2338 | 67 | 68 |
| | 66100 | −1 | GCATGGTACTGGTACCAAAA(CAG) | 2339 | 74 | 63 |
| 42 | 68003 | 1 | ACTTACCTCCTGAATACGGT(GAG) | 2340 | 90 | 67 |
| | 67959 | −1 | GGCTTTTTATTTGTATGCGG(CAG) | 2341 | 79 | 72 |
| 43 | 70978 | 1 | TTTTGAGGTCACATATGATG(GGG) | 2342 | 63 | 66 |
| | 70933 | −1 | GATGGAAAAGAGGTTAGGCA(GGG) | 2343 | 64 | 67 |
| 44 | 76923 | 1 | GACAGCTCCACCGAAACAGG(AGG) | 2344 | 76 | 65 |
| | 76882 | −1 | TTTACTCTTTCACTTTCACG(AGG) | 2345 | 61 | 62 |
| 45 | 79521 | 1 | TACCAGGCCCATCCTTCCCG(AGG) | 2346 | 68 | 64 |
| | 79497 | −1 | GGGCCTGGTAGAGTGAGTAT(GGG) | 2347 | 77 | 68 |
| 46 | 80585 | 1 | GGTTTGTATAAGTGCACCCT(AGG) | 2348 | 82 | 60 |
| | 80545 | −1 | GTATAGGCTACCATACACCT(AGG) | 2349 | 88 | 69 |
| 47 | 80599 | 1 | CACCCTAGGATGTTTGCACA(AGG) | 2350 | 73 | 60 |
| | 80545 | −1 | GTATAGGCTACCATACACCT(AGG) | 2351 | 88 | 69 |
| 48 | 89635 | 1 | ATAGGCGAGCACATGAAAAG(AGG) | 2352 | 73 | 70 |
| | 89604 | −1 | TATGCCCTTCGACACCAAGG(TGG) | 2353 | 78 | 72 |
| 49 | 89689 | 1 | TGCTAAATGTGTATCACCCG(AGG) | 2354 | 88 | 68 |
| | 89654 | −1 | TTTGGGTGGTACCTGATTGG(GGG) | 2355 | 77 | 66 |
| 50 | 89689 | 1 | TGCTAAATGTGTATCACCCG(AGG) | 2356 | 88 | 68 |
| | 89655 | −1 | ATTTGGGTGGTACCTGATTG(GGG) | 2357 | 76 | 60 |
| 51 | 90246 | 1 | ATAATTCTGCACAAATCCCC(AAG) | 2358 | 64 | 66 |
| | 90216 | −1 | TTATAAGCAGGGAGGCCTGA(GAG) | 2359 | 54 | 81 |
| 52 | 91383 | 1 | GTAACATCAGCCAAGCCAGT(AGG) | 2360 | 63 | 69 |
| | 91353 | −1 | TTACTGCTGCGTCGCTCCTG(GGG) | 2361 | 76 | 69 |
| 53 | 91402 | 1 | TAGGTCCCCACCAATGCTGC(CGG) | 2362 | 75 | 65 |
| | 91353 | −1 | TTACTGCTGCGTCGCTCCTG(GGG) | 2363 | 76 | 69 |
| 54 | 91444 | 1 | TGCTGTGGACTGCAACGGTG(TGG) | 2364 | 82 | 68 |
| | 91396 | −1 | GTTCACCGGCAGCATTGGTG(GGG) | 2365 | 78 | 62 |
| 55 | 91444 | 1 | TGCTGTGGACTGCAACGGTG(TGG) | 2366 | 82 | 68 |
| | 91382 | −1 | TTGGTGGGGACCTACTGGCT(TGG) | 2367 | 75 | 62 |
| 56 | 92524 | 1 | TGCATTACCAATATCAGCAA(GGG) | 2368 | 63 | 64 |
| | 92494 | −1 | TGCAGCATAGCATAGTGAGT(GGG) | 2369 | 73 | 65 |
| 57 | 92524 | 1 | TGCATTACCAATATCAGCAA(GGG) | 2370 | 63 | 64 |
| | 92495 | −1 | ATGCAGCATAGCATAGTGAG(TGG) | 2371 | 68 | 66 |
| 58 | 93703 | 1 | GTAAGAGACAACCATTACAG(GAG) | 2372 | 67 | 81 |
| | 93663 | −1 | GGAAGACCTCATGAACTGAG(CAG) | 2373 | 61 | 70 |
| 59 | 94847 | 1 | CCCCCACATTCCCATTGTGG(GGG) | 2374 | 66 | 64 |
| | 94784 | −1 | GACCTGGGGCATATTGTCAG(GGG) | 2375 | 77 | 69 |
| 60 | 94847 | 1 | CCCCCACATTCCCATTGTGG(GGG) | 2376 | 66 | 64 |
| | 94816 | −1 | GGGGTGAAGTGTTAGACCCA(GGG) | 2377 | 73 | 67 |
| 61 | 94847 | 1 | CCCCCACATTCCCATTGTGG(GGG) | 2378 | 66 | 64 |
| | 94806 | −1 | GTTAGACCCAGGGACCAGGT(GGG) | 2379 | 62 | 69 |
| 62 | 99426 | 1 | TAAGACTAGCCTCTATAGCA(AGG) | 2380 | 80 | 63 |
| | 99395 | −1 | TTACACATGGCTATAAGGTG(CGG) | 2381 | 76 | 62 |
| 63 | 99993 | 1 | TCTCTCATCAGGTATCCAGA(AGG) | 2382 | 70 | 69 |
| | 99943 | −1 | CCAACTGTGACAAGTCAACG(TGG) | 2383 | 86 | 75 |
| 64 | 111569 | 1 | GTTATCTCAAAGGTACCCAT(GAG) | 2384 | 74 | 71 |
| | 111513 | −1 | CACTGACTATCTCTTCAGAG(AAG) | 2385 | 59 | 67 |
| 65 | 112933 | 1 | GGAGAAGGGTATAACCTTGG(GGG) | 2386 | 72 | 66 |
| | 112878 | −1 | CCTGCATCCAATGAATGGTG(TGG) | 2387 | 70 | 68 |
| 66 | 131392 | 1 | AGACCACCGTCCAGAAAGAA(GAG) | 2388 | 55 | 69 |
| | 131349 | −1 | GGATTATTCTCTCTGGACAG(TAG) | 2389 | 61 | 70 |
| 67 | 131392 | 1 | AGACCACCGTCCAGAAAGAA(GAG) | 2390 | 55 | 69 |
| | 131352 | −1 | TCTGGATTATTCTCTCTGGA(CAG) | 2391 | 55 | 83 |
| 68 | 131411 | 1 | AGAGATGCACAATAGCCCGA(CAG) | 2392 | 86 | 91 |
| | 131349 | −1 | GGATTATTCTCTCTGGACAG(TAG) | 2393 | 61 | 70 |
| 69 | 131411 | 1 | AGAGATGCACAATAGCCCGA(CAG) | 2394 | 86 | 91 |
| | 131352 | −1 | TCTGGATTATTCTCTCTGGA(CAG) | 2395 | 55 | 83 |
| 70 | 134360 | 1 | TCAGGGTCTAGAAAGACGAA(CAG) | 2396 | 68 | 69 |
| | 134315 | −1 | TACGTTTCTGAAATTGCAAG(CAG) | 2397 | 55 | 78 |
| 71 | 135192 | 1 | TCACTGCAAACACAACCCTG(AGG) | 2398 | 65 | 77 |
| | 135158 | −1 | GCAACCTTGAGACATGAGGT(AGG) | 2399 | 70 | 64 |
| 72 | 135192 | 1 | TCACTGCAAACACAACCCTG(AGG) | 2400 | 65 | 77 |
| | 135162 | −1 | GTGAGCAACCTTGAGACATG(AGG) | 2401 | 69 | 67 |
| 73 | 138647 | 1 | GTTTACATATTATTTTAACG(AAG) | 2402 | 54 | 90 |
| | 138593 | −1 | TCCATCATCCAAGTATTCAA(CAG) | 2403 | 65 | 67 |
| 74 | 140490 | 1 | AATCATTGTAACAAGCCCTA(CAG) | 2404 | 71 | 64 |
| | 140459 | −1 | ATTTGCCACATCCAACCCCA(GAG) | 2405 | 51 | 87 |
| 75 | 140966 | 1 | TTGCTGGTGGGCACACCAGA(GAG) | 2406 | 63 | 68 |
| | 140940 | −1 | ACCAGCAAGAAAGTTCCCAC(CAG) | 2407 | 68 | 67 |
| 76 | 141112 | 1 | CTATATTCCATTCATGAAGG(CAG) | 2408 | 56 | 64 |
| | 141052 | −1 | GAGTGAGTTAGATATCACAA(GAG) | 2409 | 71 | 75 |
| 77 | 144318 | 1 | TGTAGTCTCAGCTACTAGGG(AGG) | 2410 | 60 | 74 |
| | 144285 | −1 | AGGTGAGCACCACTATGCCC(GGG) | 2411 | 78 | 62 |
| 78 | 156887 | 1 | CACTCCCATCAACAGTGTGT(AGG) | 2412 | 72 | 69 |
| | 156842 | −1 | AACCATAATGGAAGACTGTG(TGG) | 2413 | 60 | 64 |
| 79 | 161670 | 1 | GTAGCCTAATGGTTTCCATG(GGG) | 2414 | 68 | 77 |
| | 161635 | −1 | CGTTTTCCAGCCGCTCCCTG(TGG) | 2415 | 62 | 63 |
| 80 | 163858 | 1 | CATGACCCTGTAGATACTGA(AGG) | 2416 | 68 | 65 |
| | 163829 | −1 | TCATGGTTTCAGAACCCCAA(GGG) | 2417 | 65 | 70 |
| 81 | 168112 | 1 | TTTCAGTCCCCATTAATGAA(CAG) | 2418 | 59 | 64 |
| | 168072 | −1 | GCATGCCAAGAACTTGACAG(AAG) | 2419 | 62 | 95 |
| 82 | 168112 | 1 | TTTCAGTCCCCATTAATGAA(CAG) | 2420 | 59 | 64 |
| | 168075 | −1 | ACAGCATGCCAAGAACTTGA(CAG) | 2421 | 58 | 72 |
| 83 | 170725 | 1 | TTTTGGTTACTGTAGCCTTG(TAG) | 2422 | 65 | 63 |
| | 170689 | −1 | GCATGGTACTGGTACCAAAA(CAG) | 2423 | 74 | 63 |
| 84 | 180194 | 1 | CATGACCTGCCAACATGCTG(GGG) | 2424 | 65 | 63 |
| | 180140 | −1 | CAGGAGTTTGAGACTAGCCT(GGG) | 2425 | 63 | 69 |

*Searching was based on WT Cas9 PAMs, 84 solutions out of ~37,500 possible pairs shown.

### Nucleobase Editors for Use in the Invention

The methods of editing ion channel-encoding genes in neurons (e.g., DRG neurons) for pain suppression are enabled by the use of the nucleobase editors. As described herein, a nucleobase editor is a fusion protein comprising: (i) a programmable DNA binding protein domain; and (ii) a deaminase domain. Any programmable DNA binding domain may be used in the based editors.

In some embodiments, the programmable DNA binding protein domain comprises the DNA binding domain of a zinc finger nuclease (ZFN) or a transcription activator-like effector domain (TALE). In some embodiments, the programmable DNA binding protein domain may be programmed by a guide nucleotide sequence and is thus referred as a “guide nucleotide sequence-programmable DNA binding-protein domain.” In some embodiments, the guide nucleotide sequence-programmable DNA binding protein is a nuclease inactive Cas9, or dCas9. A dCas9 as used herein, encompasses a Cas9 that is completely inactive with respect to its nuclease activity, or partially inactive with respect to its nuclease activity (e.g., a Cas9 nickase). Thus, in some embodiments, the guide nucleotide sequence-programmable DNA binding protein is a Cas9 nickase. In some embodiments, the guide nucleotide sequence-programmable DNA binding protein is a nuclease inactive Cpf1. In some embodiments, the guide nucleotide sequence-programmable DNA binding protein is a nuclease inactive Argonaute.

In some embodiments, the guide nucleotide sequence-programmable DNA binding protein is a dCas9 domain. In some embodiments, the guide nucleotide sequence-programmable DNA binding protein is a Cas9 nickase. In some embodiments, the dCas9 domain comprises the amino acid sequence of SEQ ID NO: 2 or SEQ ID NO: 3. In some embodiments, the dCas9 domain comprises an amino acid sequence that is at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or at least 99.5% identical to any one of the Cas9 domains provided herein (e.g., SEQ ID NOs: 11-260), and comprises mutations corresponding to D10X (X is any amino acid except for D) and/or H840X (X is any amino acid except for H) in SEQ ID NO: 1. In some embodiments, the dCas9 domain comprises an amino acid sequence that is at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or at least 99.5% identical to any one of the Cas9 domains provided herein (e.g., SEQ ID NOs: 11-260), and comprises mutations corresponding to D10A and/or H840A in SEQ ID NO: 1. In some embodiments, the Cas9 nickase comprises an amino acid sequence that is at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or at least 99.5% identical to any one of the Cas9 domains provided herein (e.g., SEQ ID NOs: 11-260), and comprises mutations corresponding to D10X (X is any amino acid except for D) in SEQ ID NO: 1 and a histidine at the position corresponding to position 840 in SEQ ID NO: 1. In some embodiments, the Cas9 nickase comprises an amino acid sequence that is at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or at least 99.5% identical to any one of the Cas9 domains provided herein (e.g., SEQ ID NOs: 11-260), and comprises mutations corresponding to D10A in SEQ ID NO: 1 and a histidine at the position corresponding to position 840 in SEQ ID NO: 1. In some embodiments, variants or homologues of dCas9 or Cas9 nickase (e.g., variants of SEQ ID NO: 2 or SEQ ID NO: 3, respectively) are provided which are at least about 70% identical, at least about 80% identical, at least about 90% identical, at least about 95% identical, at least about 98% identical, at least about 99% identical, at least about 99.5% identical, or at least about 99.9% to SEQ ID NO: 2 or SEQ ID NO: 3, respectively, and comprises mutations corresponding to D10A and/or H840A in SEQ ID NO: 1. In some embodiments, variants of Cas9 (e.g., variants of SEQ ID NO: 2) are provided having amino acid sequences which are shorter, or longer than SEQ ID NO: 2, by about 5 amino acids, by about 10 amino acids, by about 15 amino acids, by about 20 amino acids, by about 25 amino acids, by about 30 amino acids, by about 40 amino acids, by about 50 amino acids, by about 75 amino acids, by about 100 amino acids, or more, provided that the dCas9 variants comprise mutations corresponding to D10A and/or H840A in SEQ ID NO: 1. In some embodiments, variants of Cas9 nickase (e.g., variants of SEQ ID NO: 3) are provided having amino acid sequences which are shorter, or longer, than SEQ ID NO: 3, by about 5 amino acids, by about 10 amino acids, by about 15 amino acids, by about 20 amino acids, by about 25 amino acids, by about 30 amino acids, by about 40 amino acids, by about 50 amino acids, by about 75 amino acids, by about 100 amino acids, or more, provided that the dCas9 variants comprise mutations corresponding to D10A and comprises a histidine at the position corresponding to position 840 in SEQ ID NO: 1.

Additional suitable nuclease-inactive dCas9 domains will be apparent to those of skill in the art based on this disclosure and knowledge in the field, and are within the scope of this disclosure. Such additional exemplary suitable nuclease-inactive Cas9 domains include, but are not limited to, D10A/H840A, D10A/D839A/H840A, and D10A/D839A/H840A/N863A mutant domains (See, e.g., Prashant et al., Nature Biotechnology. 2013; 31(9): 833-838, which are incorporated herein by reference), or K603R (See, e.g., Chavez et al., Nature Methods 12, 326-328, 2015, which is incorporated herein by reference.

In some embodiments, the nucleobase editors described herein comprise a Cas9 domain with decreased electrostatic interactions between the Cas9 domain and the sugar-phosphate backbone of a target DNA, as compared to a wild-type Cas9 domain. In some embodiments, a Cas9 domain comprises one or more mutations that decreases the association between the Cas9 domain and a sugar-phosphate backbone of a DNA. In some embodiments, the nucleobase editors useful in the present disclosure comprises a dCas9 (e.g., with D10A and H840A mutations) or a Cas9 nickase (e.g., with D10A mutation), wherein the dCas9 or the Cas9 nickase further comprises one or more of a N497X, a R661X, a Q695X. and/or a Q926X mutation of the amino acid sequence provided in SEQ ID NO: 1, or a corresponding mutation in any of the amino acid sequences provided in SEQ ID NOs: 11-260, wherein X is any amino acid. In some embodiments, the nucleobase editors described herein comprises a dCas9 (e.g., with D10A and H840A mutations) or a Cas9 nickase (e.g., with D10A mutation), wherein the dCas9 or the Cas9 nickase further comprises one or more of a N497A, a R661A, a Q695A, and/or a Q926A mutation of the amino acid sequence provided in SEQ ID NO: 1, or a corresponding mutation in any of the amino acid sequences provided in SEQ ID NOs: 11-260. In some embodiments, the dCas9 domain (e.g., of any of the nucleobase editors provided herein) comprises the amino acid sequence as set forth in any one of SEQ ID NOs: 2-9. In some embodiments, the nucleobase editor comprises the amino acid sequence as set forth in any one of SEQ ID NOs: 10 or 293-302.

Cas9 variant with decreased electrostatic interactions between the Cas9
and DNA backbone
DKKYSIGLAIGTNSVGWAVITDEYKVPSKKFKVLGNTDRHSIKKNLIGALLFDSGETALATRLKRTARR
RYTRRKNRICYLQEIFSNEMAKVDDSFFHRLEESFLVEEDKKHERHPIFGNIVDEVAYHEKYPTIYHLRK
KLVDSTDKADLRLIYLALAHMIKFRGHFLIEGDLNPDNSDVDKLFIQLVQTYNQLFEENPINASGVDAK
AILSARLSKSRRLENLIAQLPGEKKNGLFGNLIALSLGLTPNFKSNFDLAEDAKLQLSKDTYDDDLDNLL
AQIGDQYADLFLAAKNLSDAILLSDILRVNTEITKAPLSASMIKRYDEHHQDLTLLKALVRQQLPEKYK
EIFFDQSKNGYAGYIDGGASQEEFYKFIKPILEKMDGTEELLVKLNREDLLRKQRTFDNGSIPHQIHLGE
LHAILRRQEDFYPFLKDNREKIEKILTFRIPYYVGPLARGNSRFAWMTRKSEETITPWNFEEVVDKGASA
QSFIERMTAFDKNLPNEKVLPKHSLLYEYFTVYNELTKVKYVTEGMRKPAFLSGEQKKAIVDLLFKTN
RKVTVKQLKEDYFKKIECFDSVEISGVEDRFNASLGTYHDLLKIIKDKDFLDNEENEDILEDIVLTLTLFE
DREMIEERLKTYAHLFDDKVMKQLKRRRYTGWGALSRKLINGIRDKQSGKTILDFLKSDGFANRNFM
ALIHDDSLTFKEDIQKAQVSGQGDSLHEHIANLAGSPAIKKGILQTVKVVDELVKVMGRHKPENIVIEM
ARENQTTQKGQKNSRERMKRIEEGIKELGSQILKEHPVENTQLQNEKLYLYYLQNGRDMYVDQELDIN
RLSDYDVDHIVPQSFLKDDSIDNKVLTRSDKNRGKSDNVPSEEVVKKMKNYWRQLLNAKLITQRKFD
NLTKAERGGLSELDKAGFIKRQLVETRAITKHVAQILDSRMNTKYDENDKLIREVKVITLKSKLVSDFR
KDFQFYKVREINNYHHAHDAYLNAVVGTALIKKYPKLESEFVYGDYKVYDVRKMIAKSEQEIGKATA
KYFFYSNIMNFFKTEITLANGEIRKRPLIETNGETGEIVWDKGRDFATVRKVLSMPQVNIVKKTEVQTG
GFSKESILPKRNSDKLIARKKDWDPKKYGGFDSPTVAYSVLVVAKVEKGKSKKLKSVKELLGITIMERS
SFEKNPIDFLEAKGYKEVKKDLIIKLPKYSLFELENGRKRMLASAGELQKGNELALPSKYVNFLYLASH
YEKLKGSPEDNEQKQLFVEQHKHYLDEIIEQISEFSKRVILADANLDKVLSAYNKHRDKPIREQAENIIH
LFTLTNLGAPAAFKYFDTTIDRKRYTSTKEVLDATLIHQSITGLYETRIDLSQLGGD (SEQ ID NO: 9,
mutations relative to SEQ ID NO: 1 are bolded and underlined)
High fidelity nucleobase editor
(SEQ ID NO: 321)
msSETGPVAVDPTLRRRIEPHEFEVFFDPRELRKETCLLYEINWGGRHSIWRHTSQNTNKHVEVNFIEKF
TTERYFCPNTRCSITWFLSWSPCGECSRAITEFLSRYPHVTLFIYIARLYHHADPRNRQGLRDLISSGVTI
QIMTEQESGYCWRNFVNYSPSNEAHWPRYPHLWVRLYVLELYCIILGLPPCLNILRRKQPQLTFFTIALQ
SCHYQRLPPHILWATGLKSGSETPGTSESATPESDKKYSIGLAIGTNSVGWAVITDEYKVPSKKFKVLG
NTDRHSIKKNLIGALLFDSGETALATRLKRTARRRYTRRKNRICYLQEIFSNEMAKVDDSFFHRLEESFL
VEEDKKHERHPIFGNIVDEVAYHEKYPTIYHLRKKLVDSTDKADLRLIYLALAHMIKFRGHFLIEGDLN
PDNSDVDKLFIQLVQTYNQLFEENPINASGVDAKAILSARLSKSRRLENLIAQLPGEKKNGLFGNLIALS
LGLTPNFKSNFDLAEDAKLQLSKDTYDDDLDNLLAQIGDQYADLFLAAKNLSDAILLSDILRVNTEITK
APLSASMIKRYDEHHQDLTLLKALVRQQLPEKYKEIFFDQSKNGYAGYIDGGASQEEFYKFIKPILEKM
DGTEELLVKLNREDLLRKQRTFDNGSIPHQIHLGELHAILRRQEDFYPFLKDNREKIEKILTFRIPYYVGP
LARGNSRFAWMTRKSEETITPWNFEEVVDKGASAQSFIERMTAFDKNLPNEKVLPKHSLLYEYFTVYN
ELTKVKYVTEGMRKPAFLSGEQKKAIVDLLFKTNRKVTVKQLKEDYFKKIECFDSVEISGVEDRFNASL
GTYHDLLKIIKDKDFLDNEENEDILEDIVLTLTLFEDREMIEERLKTYAHLFDDKVMKQLKRRRYTGWG
ALSRKLINGIRDKQSGKTILDFLKSDGFANRNFMALIHDDSLTFKEDIQKAQVSGQGDSLHEHIANLAGS
PAIKKGILQTVKVVDELVKVMGRHKPENIVIEMARENQTTQKGQKNSRERMKRIEEGIKELGSQILKEH
PVENTQLQNEKLYLYYLQNGRDMYVDQELDINRLSDYDVDHIVPQSFLKDDSIDNKVLTRSDKNRGKS
DNVPSEEVVKKMKNYWRQLLNAKLITQRKFDNLTKAERGGLSELDKAGFIKRQLVETRAITKHVAQIL
DSRMNTKYDENDKLIREVKVITLKSKLVSDFRKDFQFYKVREINNYHHAHDAYLNAVVGTALIKKYPK
LESEFVYGDYKVYDVRKMIAKSEQEIGKATAKYFFYSNIMNFFKTEITLANGEIRKRPLIETNGETGEIV
WDKGRDFATVRKVLSMPQVNIVKKTEVQTGGFSKESILPKRNSDKLIARKKDWDPKKYGGFDSPTVA
YSVLVVAKVEKGKSKKLKSVKELLGITIMERSSFEKNPIDFLEAKGYKEVKKDLIIKLPKYSLFELENGR
KRMLASAGELQKGNELALPSKYVNFLYLASHYEKLKGSPEDNEQKQLFVEQHKHYLDEIIEQISEFSKR
VILADANLDKVLSAYNKHRDKPIREQAENIIHLFTLTNLGAPAAFKYFDTTIDRKRYTSTKEVLDATLIH
QSITGLYETRIDLSQLGGD

In some embodiments, the guide nucleotide sequence-programmable DNA binding protein is a single effector of a microbial CRISPR-Cas system. Single effectors of microbial CRISPR-Cas systems include, without limitation, Cas9, Cpf1, C2c1, C2c2, and C2c3. Typically, microbial CRISPR-Cas systems are divided into Class 1 and Class 2 systems. Class 1 systems have multisubunit effector complexes, while Class 2 systems have a single protein effector. Cas9 and Cpf1 are Class 2 effectors. In addition to Cas9 and Cpf1, three distinct Class 2 CRISPR-Cas systems (C2c1, C2c2, and C2c3) have been described by Shmakov et al., “Discovery and Functional Characterization of Diverse Class 2 CRISPR Cas Systems”, Mol. Cell, 2015 Nov. 5; 60(3): 385-397, the entire contents of which are herein incorporated by reference. Effectors of two of the systems, C2c1 and C2c3, contain RuvC-like endonuclease domains related to Cpf1. A third system, C2c2 contains an effector with two predicted HEPN RNase domains. Production of mature CRISPR RNA is tracrRNA-independent, unlike production of CRISPR RNA by C2c1. C2c1 depends on both CRISPR RNA and tracrRNA for DNA cleavage. Bacterial C2c2 has been shown to possess a unique RNase activity for CRISPR RNA maturation distinct from its RNA-activated single-stranded RNA degradation activity. These RNase functions are different from each other and from the CRISPR RNA-processing behavior of Cpf1. See, e.g., East-Seletsky, et al., “Two distinct RNase activities of CRISPR-C2c2 enable guide-RNA processing and RNA detection”, Nature, 2016 Oct. 13; 538(7624):270-273, the entire contents of which are hereby incorporated by reference. In vitro biochemical analysis of C2c2 in Leptotrichia shahii has shown that C2c2 is guided by a single CRISPR RNA and can be programmed to cleave ssRNA targets carrying complementary protospacers. Catalytic residues in the two conserved HEPN domains mediate cleavage. Mutations in the catalytic residues generate catalytically inactive RNA-binding proteins. See e.g., Abudayyeh et al., “C2c2 is a single-component programmable RNA-guided RNA-targeting CRISPR effector,” Science, 2016 Aug. 5; 353(6299), the entire contents of which are hereby incorporated by reference.

The crystal structure of Alicyclobaccillus acidoterrastris C2c1 (AacC2c1) has been reported in complex with a chimeric single-molecule guide RNA (sgRNA). See, e.g., Liu et al., “C2c1-sgRNA Complex Structure Reveals RNA-Guided DNA Cleavage Mechanism”, Mol. Cell, 2017 Jan. 19; 65(2):310-322, incorporated herein by reference. The crystal structure has also been reported for Alicyclobacillus acidoterrestris C2c1 bound to target DNAs as ternary complexes. See, e.g., Yang et al., “PAM-dependent Target DNA Recognition and Cleavage by C2C1 CRISPR-Cas endonuclease”, Cell, 2016 Dec. 15; 167(7):1814-1828, the entire contents of which are hereby incorporated by reference. Catalytically competent conformations of AacC2c1, both with target and non-target DNA strands, have been captured independently positioned within a single RuvC catalytic pocket, with C2c1-mediated cleavage resulting in a staggered seven-nucleotide break of target DNA. Structural comparisons between C2c1 ternary complexes and previously identified Cas9 and Cpf1 counterparts demonstrate the diversity of mechanisms used by CRISPR-Cas9 systems.

In some embodiments, the nucleobase editors described herein comprise a C2c1, a C2c2, or a C2c3 protein. In some embodiments, the guide nucleotide sequence-programmable DNA binding protein is a C2c1 protein. In some embodiments, the guide nucleotide sequence-programmable DNA binding protein is a C2c2 protein. In some embodiments, the guide nucleotide sequence-programmable DNA binding protein is a C2c3 protein. In some embodiments, the guide nucleotide sequence-programmable DNA binding protein comprises an amino acid sequence that is at least 85%, at least 90%, at least 91%, at least 92%, at least 93%, at least 94%, at least 95%, at least 96%, at least 97%, at least 98%, at least 99%, or at least 99.5% identical to a naturally-occurring C2c1, C2c2, or C2c3 protein. In some embodiments, the guide nucleotide sequence-programmable DNA binding protein is a naturally-occurring C2c1, C2c2, or C2c3 protein. In some embodiments, the guide nucleotide sequence-programmable DNA binding protein comprises an amino acid sequence that is at least 85%, at least 90%, at least 91%, at least 92%, at least 93%, at least 94%, at least 95%, at least 96%, at least 97%, at least 98%, at least 99%, or at least 99.5% identical to any one of SEQ ID NOs: 756-758. In some embodiments, the guide nucleotide sequence-programmable DNA binding protein comprises an amino acid sequence of any one SEQ ID NOs: 756-758. It should be appreciated that C2c1, C2c2, or C2c3 from other bacterial species may also be used in accordance with the present disclosure.

C2c1 (uniprot.org/uniprot/T0D7A2#)
sp|T0D7A2|C2C1_ALIAG CRISPR-associated endonuclease C2c1 OS = Alicyclobacillus
acidoterrestris (strain ATCC 49025 / DSM 3922 / CIP 106132 / NCIMB 13137 / GD3B)
GN = c2c1 PE = 1 SV = 1
(SEQ ID NO: 2470)
MAVKSIKVKLRLDDMPEIRAGLWKLHKEVNAGVRYYTEWLSLLRQENLYRRSPNGDGEQECDKTAE
ECKAELLERLRARQVENGHRGPAGSDDELLQLARQLYELLVPQAIGAKGDAQQIARKFLSPLADKDAV
GGLGIAKAGNKPRWVRMREAGEPGWEEEKEKAETRKSADRTADVLRALADFGLKPLMRVYTDSEMS
SVEWKPLRKGQAVRTWDRDMFQQAIERMMSWESWNQRVGQEYAKLVEQKNRFEQKNFVGQEHLV
HLVNQLQQDMKEASPGLESKEQTAHYVTGRALRGSDKVFEKWGKLAPDAPFDLYDALIKNVQRRNT
RRFGSHDLFAKLAEPEYQALWREDASFLTRYAVYNSILRKLNHAKMFATFTLPDATAHPIWTRFDKLG
GNLHQYTFLFNEFGERRHAIRFHKLLKVENGVAREVDDVTVPISMSEQLDNLLPRDPNEPIALYFRDYG
AEQHFTGEFGGAKIQCRRDQLAHMHRRRGARDVYLNVSVRVQSQSEARGERRPPYAAVFRLVGDNH
RAFVHFDKLSDYLAEHPDDGKLGSEGLLSGLRVMSVDLGLRTSASISVFRVARKDELKPNSKGRVPFFF
PIKGNDNLVAVHERSQLLKLPGETESKDLRAIREERQRTLRQLRTQLAYLRLLVRCGSEDVGRRERSW
AKLIEQPVDAANHMTPDWREAFENELQKLKSLHGICSDKEWMDAVYESVRRVWRHMGKQVRDWRK
DVRSGERPKIRGYAKDVVGGNSIEQIEYLERQYKFLKSWSFFGKVSGQVIRAEKGSRFAITLREHIDHAK
EDRLKKLADRIIMEALGYVYALDERGKGKWVAKYPPCQLILLEELSEYQFNNDRPPSENNQLMQWSH
RGVFQELINQAQVHDLLVGTMYAAFSSRFDARTGAPGIRCRRVPARCTQEHNPEPFPWWLNKFVVEHT
LDACPLRADDLIPTGEGEIFVSPFSAEEGDFHQIHADLNAAQNLQQRLWSDFDISQIRERCDWGEVDGE
LVLIPRLTGKRTADSYSNKVFYTNTGVTYYERERGKKRRKVFAQEKLSEEEAELLVEADEAREKSVVL
MRDPSGIINRGNWTRQKEFWSMVNQRIEGYLVKQIRSRVPLQDSACENTGDI
C2c2 (uniprot.org/uniprot/P0DOC6)
>sp|P0DOC6|C2C2_LEPSD CRISPR-associated endoribonuclease C2c2 OS = Leptotrichia
shahii (strain DSM 19757 / CCUG 47503 / CIP 107916 / JCM 16776 / LB37) GN = c2c2
PE = 1 SV = 1
(SEQ ID NO: 2471)
MGNLFGHKRWYEVRDKKDFKIKRKVKVKRNYDGNKYILNINENNNKEKIDNNKFIRKYINYKKNDNI
LKEFTRKFHAGNILFKLKGKEGIIRIENNDDFLETEEVVLYIEAYGKSEKLKALGITKKKIIDEAIRQGITK
DDKKIEIKRQENEEEIEIDIRDEYTNKTLNDCSIILRIIENDELETKKSIYEIFKNINMSLYKIIEKIIENETEK
VFENRYYEEHLREKLLKDDKIDVILTNFMEIREKIKSNLEILGFVKFYLNVGGDKKKSKNKKMLVEKIL
NINVDLTVEDIADFVIKELEFWNITKRIEKVKKVNNEFLEKRRNRTYIKSYVLLDKHEKFKIERENKKDK
IVKFFVENIKNNSIKEKIEKILAEFKIDELIKKLEKELKKGNCDTEIFGIFKKHYKVNFDSKKFSKKSDEEK
ELYKIIYRYLKGRIEKILVNEQKVRLKKMEKIEIEKILNESILSEKILKRVKQYTLEHIMYLGKLRHNDID
MTTVNTDDFSRLHAKEELDLELITFFASTNMELNKIFSRENINNDENIDFFGGDREKNYVLDKKILNSKI
KIIRDLDFIDNKNNITNNFIRKFTKIGTNERNRILHAISKERDLQGTQDDYNKVINIIQNLKISDEEVSKAL
NLDVVFKDKKNIITKINDIKISEENNNDIKYLPSFSKVLPEILNLYRNNPKNEPFDTIETEKIVLNALIYVN
KELYKKLILEDDLEENESKNIFLQELKKTLGNIDEIDENIIENYYKNAQISASKGNNKAIKKYQKKVIECY
IGYLRKNYEELFDFSDFKMNIQEIKKQIKDINDNKTYERITVKTSDKTIVINDDFEYIISIFALLNSNAVIN
KIRNRFFATSVWLNTSEYQNIIDILDEIMQLNTLRNECITENWNLNLEEFIQKMKEIEKDFDDFKIQTKKE
IFNNYYEDIKNNILTEFKDDINGCDVLEKKLEKIVIFDDETKFEIDKKSNILQDEQRKLSNINKKDLKKKV
DQYIKDKDQEIKSKILCRIIFNSDFLKKYKKEIDNLIEDMESENENKFQEIYYPKERKNELYIYKKNLFLNI
GNPNFDKIYGLISNDIKMADAKFLFNIDGKNIRKNKISEIDAILKNLNDKLNGYSKEYKEKYIKKLKEND
DFFAKNIQNKNYKSFEKDYNRVSEYKKIRDLVEFNYLNKIESYLIDINWKLAIQMARFERDMHYIVNGL
RELGIIKLSGYNTGISRAYPKRNGSDGFYTTTAYYKFFDEESYKKFEKICYGFGIDLSENSEINKPENESIR
NYISHFYIVRNPFADYSIAEQIDRVSNLLSYSTRYNNSTYASVFEVFKKDVNLDYDELKKKFKLIGNNDI
LERLMKPKKVSVLELESYNSDYIKNLIIELLTKIENTNDTL
C2c3, translated from >CEPX01008730.1 marine metagenome genome assembly
TARA_037_MES_0.1-0.22, contig TARA_037_MES_0.1-0.22_scaffold22115_1, whole
genome shotgun sequence.
(SEQ ID NO: 2472)
MRSNYHGGRNARQWRKQISGLARRTKETVFTYKFPLETDAAEIDFDKAVQTYGIAEGVGHGSLIGLVC
AFHLSGFRLFSKAGEAMAFRNRSRYPTDAFAEKLSAIMGIQLPTLSPEGLDLIFQSPPRSRDGIAPVWSE
NEVRNRLYTNWTGRGPANKPDEHLLEIAGEIAKQVFPKFGGWDDLASDPDKALAAADKYFQSQGDFP
SIASLPAAIMLSPANSTVDFEGDYIAIDPAAETLLHQAVSRCAARLGRERPDLDQNKGPFVSSLQDALVS
SQNNGLSWLFGVGFQHWKEKSPKELIDEYKVPADQHGAVTQVKSFVDAIPLNPLFDTTHYGEFRASVA
GKVRSWVANYWKRLLDLKSLLATTEFTLPESISDPKAVSLFSGLLVDPQGLKKVADSLPARLVSAEEAI
DRLMGVGIPTAADIAQVERVADEIGAFIGQVQQFNNQVKQKLENLQDADDEEFLKGLKIELPSGDKEPP
AINRISGGAPDAAAEISELEEKLQRLLDARSEHFQTISEWAEENAVTLDPIAAMVELERLRLAERGATGD
PEEYALRLLLQRIGRLANRVSPVSAGSIRELLKPVFMEEREFNLFFHNRLGSLYRSPYSTSRHQPFSIDVG
KAKAIDWIAGLDQISSDIEKALSGAGEALGDQLRDWINLAGFAISQRLRGLPDTVPNALAQVRCPDDVR
IPPLLAMLLEEDDIARDVCLKAFNLYVSAINGCLFGALREGFIVRTRFQRIGTDQIHYVPKDKAWEYPDR
LNTAKGPINAAVSSDWIEKDGAVIKPVETVRNLSSTGFAGAGVSEYLVQAPHDWYTPLDLRDVAHLVT
GLPVEKNITKLKRLTNRTAFRMVGASSFKTHLDSVLLSDKIKLGDFTIIIDQHYRQSVTYGGKVKISYEP
ERLQVEAAVPVVDTRDRTVPEPDTLFDHIVAIDLGERSVGFAVFDIKSCLRTGEVKPIHDNNGNPVVGT
VAVPSIRRLMKAVRSHRRRRQPNQKVNQTYSTALQNYRENVIGDVCNRIDTLMERYNAFPVLEFQIKN
FQAGAKQLEIVYGS

The Cas9 protein recognizes a short motif (PAM motif) in the CRISPR repeat sequences in the target DNA sequence. A “PAM motif.” or “protospacer adjacent motif.” as used herein, refers to a DNA sequence immediately following the DNA sequence targeted by the Cas9 nuclease in the CRISPR bacterial adaptive immune system. PAM is a component of the invading virus or plasmid but is not a component of the bacterial CRISPR locus. Wild-type Streptococcus pyogenes Cas9 recognizes a canonical PAM sequence (5′-NGG-3′). Other Cas9 nucleases (e.g., Cas9 from Streptococcus thermophiles, Staphylococcus aureus, Neisseria meningitidis, or Treponema denticolaor) and Cas9 variants thereof have been described in the art to have different, or more relaxed PAM requirements. For example, in Kleinstiver et al., Nature 523, 481-485, 2015; Klenstiver et al., Nature 529, 490-495, 2016; Ran et al., Nature, April 9; 520(7546): 186-191, 2015; Kleinstiver et al., Nat Biotechnol, 33(12):1293-1298, 2015; Hou et al., Proc Natl Acad Sci USA, 110(39):15644-9, 2014; Prykhozhij et al., PLoS One, 10(3): e0119372, 2015: Zetsche et al., Cell 163, 759-771, 2015; Gao et al., Nature Biotechnology, doi:10.1038/nbt.3547, 2016; Want et al., Nature 461, 754-761, 2009; Chavez et al., doi: dx.doi.org/10.1101/058974; Fagerlund et al., Genome Biol. 2015; 16: 25, 2015; Zetsche et al., Cell, 163, 759-771, 2015; and Swarts et al., Nat Struct Mol Biol. 21(9):743-53, 2014, each of which is incorporated herein by reference.

Thus, the guide nucleotide sequence-programmable DNA-binding proteins useful in the present disclosure may recognize a variety of PAM sequences including, without limitation: NGG, NGAN, NGNG, NGAG, NGCG, NNGRRT, NGRRN, NNNRRT, NNNGATT, NNAGAAW, NAAAC, TTN, TTTN, and YTN, wherein Y is a pyrimidine, and N is any nucleobase. In some embodiments, the PAM is located 3′ of the target base. In some embodiments, the PAM is located 5′ of the target base.

One example of an RNA-programmable DNA-binding protein that has different PAM specificity is Clustered Regularly Interspaced Short Palindromic Repeats from Prevotella and Francisella 1 (Cpf1). Similar to Cas9, Cpf1 is also a class 2 CRISPR effector. It has been shown that Cpf1 mediates robust DNA interference with features distinct from Cas9. Cpf1 is a single RNA-guided endonuclease lacking tracrRNA, and it utilizes a T-rich protospacer-adjacent motif (TTN, TTTN, or YTN). Moreover, Cpf1 cleaves DNA via a staggered DNA double-stranded break. Out of 16 Cpf1-family proteins, two enzymes from Acidaminococcus and Lachnospiraceae are shown to have efficient genome-editing activity in human cells.

Also useful in the present disclosure are nuclease-inactive Cpf1 (dCpf1) variants that may be used as a guide nucleotide sequence-programmable DNA-binding protein domain. The Cpf1 protein has a RuvC-like endonuclease domain that is similar to the RuvC domain of Cas9 but does not have a HNH endonuclease domain, and the N-terminal of Cpf1 does not have the alfa-helical recognition lobe of Cas9. It was shown in Zetsche et al., Cell, 163, 759-771, 2015 (which is incorporated herein by reference) that, the RuvC-like domain of Cpf1 is responsible for cleaving both DNA strands and inactivation of the RuvC-like domain inactivates Cpf1 nuclease activity. For example, mutations corresponding to D917A, E1006A, or D1255A in Francisella novicida Cpf1 (SEQ ID NO: 10) inactivates Cpf1 nuclease activity. In some embodiments, the dCpf1 of the present disclosure comprises mutations corresponding to D917A, E1006A, D1255A, D917A/E1006A, D917A/D1255A, E1006A/D1255A, or D917A/E1006A/D1255A in SEQ ID NO: 10. It is to be understood that any mutations, e.g., substitution mutations, deletions, or insertions that inactivate the RuvC domain of Cpf1 may be used in accordance with the present disclosure.

Thus, in some embodiments, the guide nucleotide sequence-programmable DNA binding protein is a nuclease inactive Cpf1 (dCpf1). In some embodiments, the dCpf1 comprises the amino acid sequence of any one SEQ ID NOs: 261-267. In some embodiments, the dCpf1 comprises an amino acid sequence that is at least 85%, at least 90%, at least 91%, at least 92%, at least 93%, at least 94%, at least 95%, at least 96%, at least 97%, at least 98%, at least 99%, or at least 99.5% identical to SEQ ID NO: 10, and comprises mutations corresponding to D917A. E1006A, D1255A, D917A/E1006A, D917A/D1255A, E1006A/D1255A. or D917A/E1006A/D1255A in SEQ ID NO: 10. Cpf1 from other bacterial species may also be used in accordance with the present disclosure.

Wild type Francisella novicida Cpf1 (D917, E1006, and D1255 are
bolded and underlined)
(SEQ ID NO: 10)
MSIYQEFVNKYSLSKTLRFELIPQGKTLENIKARGLILDDEKRAKDYKKAKQIIDKYHQFFIEEILSSVCIS
EDLLQNYSDVYFKLKKSDDDNLQKDFKSAKDTIKKQISEYIKDSEKFKNLFNQNLIDAKKGQESDLILW
LKQSKDNGIELFKANSDITDIDEALEIIKSFKGWTTYFKGFHENRKNVYSSNDIPTSIIYRIVDDNLPKFLE
NKAKYESLKDKAPEAINYEQIKKDLAELLTFDIDYKTSEVNQRVFSLDEVFEIANFNNYLNQSGITKFNT
IIGGKFVNGENTKRKGINEYINLYSQQINDKTLKKYKMSVLFKQILSDTESKSFVIDKLEDDSDVVTTMQ
SFYEQIAAFKTVEEKSIKETLSLLFDDLKAQKLDLSKIYFKNDKSLTDLSQQVFDDYSVIGTAVLEYITQ
QIAPKNLDNPSKKEQELIAKKTEKAKYLSLETIKLALEEFNKHRDIDKQCRFEEILANFAAIPMIFDEIAQ
NKDNLAQISIKYQNQGKKDLLQASAEDDVKAIKDLLDQTNNLLHKLKIFHISQSEDKANILDKDEHFYL
VFEECYFELANIVPLYNKIRNYITQKPYSDEKFKLNFENSTLANGWDKNKEPDNTAILFIKDDKYYLGV
MNKKNNKIFDDKAIKENKGEGYKKIVYKLLPGANKMLPKVFFSAKSIKFYNPSEDILRIRNHSTHTKNG
SPQKGYEKFEFNIEDCRKFIDFYKQSISKHPEWKDFGFRFSDTQRYNSIDEFYREVENQGYKLTFENISES
YIDSVVNQGKLYLFQIYNKDFSAYSKGRPNLHTLYWKALFDERNLQDVVYKLNGEAELFYRKQSIPKK
ITHPAKEAIANKNKDNPKKESVFEYDLIKDKRFTEDKFFFHCPITINFKSSGANKFNDEINLLLKEKAND
VHILSIDRGERHLAYYTLVDGKGNIIKQDTFNIIGNDRMKTNYHDKLAAIEKDRDSARKDWKKINNIKE
MKEGYLSQVVHEIAKLVIEYNAIVVFEDLNFGFKRGRFKVEKQVYQKLEKMLIEKLNYLVFKDNEFDK
TGGVLRAYQLTAPFETFKKMGKQTGIIYYVPAGFTSKICPVTGFVNQLYPKYESVSKSQEFFSKFDKICY
NLDKGYFEFSFDYKNFGDKAAKGKWTIASFGSRLINFRNSDKNHNWDTREVYPTKELEKLLKDYSIEY
GHGECIKAAICGESDKKFFAKLTSVLNTILQMRNSKTGTELDYLISPVADVNGNFFDSRQAPKNMPQDA
DANGAYHIGLKGLMLLGRIKNNQEGKKLNLVIKNEEYFEFVQNRNN
Francisella novicida Cpf1 D917A (A917, E1006, and D1255 are
bolded and underlined)
(SEQ ID NO: 261)
MSIYQEFVNKYSLSKTLRFELIPQGKTLENIKARGLILDDEKRAKDYKKAKQIIDKYHQFFIEEILSSVCIS
EDLLQNYSDVYFKLKKSDDDNLQKDFKSAKDTIKKQISEYIKDSEKFKNLFNQNLIDAKKGQESDLILW
LKQSKDNGIELFKANSDITDIDEALEIIKSFKGWTTYFKGFHENRKNVYSSNDIPTSIIYRIVDDNLPKFLE
NKAKYESLKDKAPEAINYEQIKKDLAELLTFDIDYKTSEVNQRVFSLDEVFEIANFNNYLNQSGITKFNT
IIGGKFVNGENTKRKGINEYINLYSQQINDKTLKKYKMSVLFKQILSDTESKSFVIDKLEDDSDVVTTMQ
SFYEQIAAFKTVEEKSIKETLSLLFDDLKAQKLDLSKIYFKNDKSLTDLSQQVFDDYSVIGTAVLEYITQ
QIAPKNLDNPSKKEQELIAKKTEKAKYLSLETIKLALEEFNKHRDIDKQCRFEEILANFAAIPMIFDEIAQ
NKDNLAQISIKYQNQGKKDLLQASAEDDVKAIKDLLDQTNNLLHKLKIFHISQSEDKANILDKDEHFYL
VFEECYFELANIVPLYNKIRNYITQKPYSDEKFKLNFENSTLANGWDKNKEPDNTAILFIKDDKYYLGV
MNKKNNKIFDDKAIKENKGEGYKKIVYKLLPGANKMLPKVFFSAKSIKFYNPSEDILRIRNHSTHTKNG
SPQKGYEKFEFNIEDCRKFIDFYKQSISKHPEWKDFGFRFSDTQRYNSIDEFYREVENQGYKLTFENISES
YIDSVVNQGKLYLFQIYNKDFSAYSKGRPNLHTLYWKALFDERNLQDVVYKLNGEAELFYRKQSIPKK
ITHPAKEAIANKNKDNPKKESVFEYDLIKDKRFTEDKFFFHCPITINFKSSGANKFNDEINLLLKEKAND
VHILSIARGERHLAYYTLVDGKGNIIKQDTFNIIGNDRMKTNYHDKLAAIEKDRDSARKDWKKINNIKE
MKEGYLSQVVHEIAKLVIEYNAIVVFEDLNFGFKRGRFKVEKQVYQKLEKMLIEKLNYLVFKDNEFDK
TGGVLRAYQLTAPFETFKKMGKQTGIIYYVPAGFTSKICPVTGFVNQLYPKYESVSKSQEFFSKFDKICY
NLDKGYFEFSFDYKNFGDKAAKGKWTIASFGSRLINFRNSDKNHNWDTREVYPTKELEKLLKDYSIEY
GHGECIKAAICGESDKKFFAKLTSVLNTILQMRNSKTGTELDYLISPVADVNGNFFDSRQAPKNMPQDA
DANGAYHIGLKGLMLLGRIKNNQEGKKLNLVIKNEEYFEFVQNRNN
Francisella novicida Cpf1 E1006A (D917, A1006, and D1255 are
bolded and underlined)
(SEQ ID NO: 262)
MSIYQEFVNKYSLSKTLRFELIPQGKTLENIKARGLILDDEKRAKDYKKAKQIIDKYHQFFIEEILSSVCIS
EDLLQNYSDVYFKLKKSDDDNLQKDFKSAKDTIKKQISEYIKDSEKFKNLFNQNLIDAKKGQESDLILW
LKQSKDNGIELFKANSDITDIDEALEIIKSFKGWTTYFKGFHENRKNVYSSNDIPTSIIYRIVDDNLPKFLE
NKAKYESLKDKAPEAINYEQIKKDLAELLTFDIDYKTSEVNQRVFSLDEVFEIANFNNYLNQSGITKFNT
IIGGKFVNGENTKRKGINEYINLYSQQINDKTLKKYKMSVLFKQILSDTESKSFVIDKLEDDSDVVTTMQ
SFYEQIAAFKTVEEKSIKETLSLLFDDLKAQKLDLSKIYFKNDKSLTDLSQQVFDDYSVIGTAVLEYITQ
QIAPKNLDNPSKKEQELIAKKTEKAKYLSLETIKLALEEFNKHRDIDKQCRFEEILANFAAIPMIFDEIAQ
NKDNLAQISIKYQNQGKKDLLQASAEDDVKAIKDLLDQTNNLLHKLKIFHISQSEDKANILDKDEHFYL
VFEECYFELANIVPLYNKIRNYITQKPYSDEKFKLNFENSTLANGWDKNKEPDNTAILFIKDDKYYLGV
MNKKNNKIFDDKAIKENKGEGYKKIVYKLLPGANKMLPKVFFSAKSIKFYNPSEDILRIRNHSTHTKNG
SPQKGYEKFEFNIEDCRKFIDFYKQSISKHPEWKDFGFRFSDTQRYNSIDEFYREVENQGYKLTFENISES
YIDSVVNQGKLYLFQIYNKDFSAYSKGRPNLHTLYWKALFDERNLQDVVYKLNGEAELFYRKQSIPKK
ITHPAKEAIANKNKDNPKKESVFEYDLIKDKRFTEDKFFFHCPITINFKSSGANKFNDEINLLLKEKAND
VHILSIDRGERHLAYYTLVDGKGNIIKQDTFNIIGNDRMKTNYHDKLAAIEKDRDSARKDWKKINNIKE
MKEGYLSQVVHEIAKLVIEYNAIVVFADLNFGFKRGRFKVEKQVYQKLEKMLIEKLNYLVFKDNEFDK
TGGVLRAYQLTAPFETFKKMGKQTGIIYYVPAGFTSKICPVTGFVNQLYPKYESVSKSQEFFSKFDKICY
NLDKGYFEFSFDYKNFGDKAAKGKWTIASFGSRLINFRNSDKNHNWDTREVYPTKELEKLLKDYSIEY
GHGECIKAAICGESDKKFFAKLTSVLNTILQMRNSKTGTELDYLISPVADVNGNFFDSRQAPKNMPQDA
DANGAYHIGLKGLMLLGRIKNNQEGKKLNLVIKNEEYFEFVQNRNN
Francisella novicida Cpf1 D1255A (D917, E1006, and A1255 are
bolded and underlined)
(SEQ ID NO: 263)
MSIYQEFVNKYSLSKTLRFELIPQGKTLENIKARGLILDDEKRAKDYKKAKQIIDKYHQFFIEEILSSVCIS
EDLLQNYSDVYFKLKKSDDDNLQKDFKSAKDTIKKQISEYIKDSEKFKNLFNQNLIDAKKGQESDLILW
LKQSKDNGIELFKANSDITDIDEALEIIKSFKGWTTYFKGFHENRKNVYSSNDIPTSIIYRIVDDNLPKFLE
NKAKYESLKDKAPEAINYEQIKKDLAELLTFDIDYKTSEVNQRVFSLDEVFEIANFNNYLNQSGITKFNT
IIGGKFVNGENTKRKGINEYINLYSQQINDKTLKKYKMSVLFKQILSDTESKSFVIDKLEDDSDVVTTMQ
SFYEQIAAFKTVEEKSIKETLSLLFDDLKAQKLDLSKIYFKNDKSLTDLSQQVFDDYSVIGTAVLEYITQ
QIAPKNLDNPSKKEQELIAKKTEKAKYLSLETIKLALEEFNKHRDIDKQCRFEEILANFAAIPMIFDEIAQ
NKDNLAQISIKYQNQGKKDLLQASAEDDVKAIKDLLDQTNNLLHKLKIFHISQSEDKANILDKDEHFYL
VFEECYFELANIVPLYNKIRNYITQKPYSDEKFKLNFENSTLANGWDKNKEPDNTAILFIKDDKYYLGV
MNKKNNKIFDDKAIKENKGEGYKKIVYKLLPGANKMLPKVFFSAKSIKFYNPSEDILRIRNHSTHTKNG
SPQKGYEKFEFNIEDCRKFIDFYKQSISKHPEWKDFGFRFSDTQRYNSIDEFYREVENQGYKLTFENISES
YIDSVVNQGKLYLFQIYNKDFSAYSKGRPNLHTLYWKALFDERNLQDVVYKLNGEAELFYRKQSIPKK
ITHPAKEAIANKNKDNPKKESVFEYDLIKDKRFTEDKFFFHCPITINFKSSGANKFNDEINLLLKEKAND
VHILSIDRGERHLAYYTLVDGKGNIIKQDTFNIIGNDRMKTNYHDKLAAIEKDRDSARKDWKKINNIKE
MKEGYLSQVVHEIAKLVIEYNAIVVFEDLNFGFKRGRFKVEKQVYQKLEKMLIEKLNYLVFKDNEFDK
TGGVLRAYQLTAPFETFKKMGKQTGIIYYVPAGFTSKICPVTGFVNQLYPKYESVSKSQEFFSKFDKICY
NLDKGYFEFSFDYKNFGDKAAKGKWTIASFGSRLINFRNSDKNHNWDTREVYPTKELEKLLKDYSIEY
GHGECIKAAICGESDKKFFAKLTSVLNTILQMRNSKTGTELDYLISPVADVNGNFFDSRQAPKNMPQDA
AANGAYHIGLKGLMLLGRIKNNQEGKKLNLVIKNEEYFEFVQNRNN
Francisella novicida Cpf1 D917A/E1006A (A917, A1006, and
D1255 are bolded and underlined)
(SEQ ID NO: 264)
MSIYQEFVNKYSLSKTLRFELIPQGKTLENIKARGLILDDEKRAKDYKKAKQIIDKYHQFFIEEILSSVCIS
EDLLQNYSDVYFKLKKSDDDNLQKDFKSAKDTIKKQISEYIKDSEKFKNLFNQNLIDAKKGQESDLILW
LKQSKDNGIELFKANSDITDIDEALEIIKSFKGWTTYFKGFHENRKNVYSSNDIPTSIIYRIVDDNLPKFLE
NKAKYESLKDKAPEAINYEQIKKDLAELLTFDIDYKTSEVNQRVFSLDEVFEIANFNNYLNQSGITKFNT
IIGGKFVNGENTKRKGINEYINLYSQQINDKTLKKYKMSVLFKQILSDTESKSFVIDKLEDDSDVVTTMQ
SFYEQIAAFKTVEEKSIKETLSLLFDDLKAQKLDLSKIYFKNDKSLTDLSQQVFDDYSVIGTAVLEYITQ
QIAPKNLDNPSKKEQELIAKKTEKAKYLSLETIKLALEEFNKHRDIDKQCRFEEILANFAAIPMIFDEIAQ
NKDNLAQISIKYQNQGKKDLLQASAEDDVKAIKDLLDQTNNLLHKLKIFHISQSEDKANILDKDEHFYL
VFEECYFELANIVPLYNKIRNYITQKPYSDEKFKLNFENSTLANGWDKNKEPDNTAILFIKDDKYYLGV
MNKKNNKIFDDKAIKENKGEGYKKIVYKLLPGANKMLPKVFFSAKSIKFYNPSEDILRIRNHSTHTKNG
SPQKGYEKFEFNIEDCRKFIDFYKQSISKHPEWKDFGFRFSDTQRYNSIDEFYREVENQGYKLTFENISES
YIDSVVNQGKLYLFQIYNKDFSAYSKGRPNLHTLYWKALFDERNLQDVVYKLNGEAELFYRKQSIPKK
ITHPAKEAIANKNKDNPKKESVFEYDLIKDKRFTEDKFFFHCPITINFKSSGANKFNDEINLLLKEKAND
VHILSIARGERHLAYYTLVDGKGNIIKQDTFNIIGNDRMKTNYHDKLAAIEKDRDSARKDWKKINNIKE
MKEGYLSQVVHEIAKLVIEYNAIVVFADLNFGFKRGRFKVEKQVYQKLEKMLIEKLNYLVFKDNEFDK
TGGVLRAYQLTAPFETFKKMGKQTGIIYYVPAGFTSKICPVTGFVNQLYPKYESVSKSQEFFSKFDKICY
NLDKGYFEFSFDYKNFGDKAAKGKWTIASFGSRLINFRNSDKNHNWDTREVYPTKELEKLLKDYSIEY
GHGECIKAAICGESDKKFFAKLTSVLNTILQMRNSKTGTELDYLISPVADVNGNFFDSRQAPKNMPQDA
DANGAYHIGLKGLMLLGRIKNNQEGKKLNLVIKNEEYFEFVQNRNN
Francisella novicida Cpf1 D917A/D1255A (A917, E1006, and
A1255 are bolded and underlined)
(SEQ ID NO: 265)
MSIYQEFVNKYSLSKTLRFELIPQGKTLENIKARGLILDDEKRAKDYKKAKQIIDKYHQFFIEEILSSVCIS
EDLLQNYSDVYFKLKKSDDDNLQKDFKSAKDTIKKQISEYIKDSEKFKNLFNQNLIDAKKGQESDLILW
LKQSKDNGIELFKANSDITDIDEALEIIKSFKGWTTYFKGFHENRKNVYSSNDIPTSIIYRIVDDNLPKFLE
NKAKYESLKDKAPEAINYEQIKKDLAELLTFDIDYKTSEVNQRVFSLDEVFEIANFNNYLNQSGITKFNT
IIGGKFVNGENTKRKGINEYINLYSQQINDKTLKKYKMSVLFKQILSDTESKSFVIDKLEDDSDVVTTMQ
SFYEQIAAFKTVEEKSIKETLSLLFDDLKAQKLDLSKIYFKNDKSLTDLSQQVFDDYSVIGTAVLEYITQ
QIAPKNLDNPSKKEQELIAKKTEKAKYLSLETIKLALEEFNKHRDIDKQCRFEEILANFAAIPMIFDEIAQ
NKDNLAQISIKYQNQGKKDLLQASAEDDVKAIKDLLDQTNNLLHKLKIFHISQSEDKANILDKDEHFYL
VFEECYFELANIVPLYNKIRNYITQKPYSDEKFKLNFENSTLANGWDKNKEPDNTAILFIKDDKYYLGV
MNKKNNKIFDDKAIKENKGEGYKKIVYKLLPGANKMLPKVFFSAKSIKFYNPSEDILRIRNHSTHTKNG
SPQKGYEKFEFNIEDCRKFIDFYKQSISKHPEWKDFGFRFSDTQRYNSIDEFYREVENQGYKLTFENISES
YIDSVVNQGKLYLFQIYNKDFSAYSKGRPNLHTLYWKALFDERNLQDVVYKLNGEAELFYRKQSIPKK
ITHPAKEAIANKNKDNPKKESVFEYDLIKDKRFTEDKFFFHCPITINFKSSGANKFNDEINLLLKEKAND
VHILSIARGERHLAYYTLVDGKGNIIKQDTFNIIGNDRMKTNYHDKLAAIEKDRDSARKDWKKINNIKE
MKEGYLSQVVHEIAKLVIEYNAIVVFEDLNFGFKRGRFKVEKQVYQKLEKMLIEKLNYLVFKDNEFDK
TGGVLRAYQLTAPFETFKKMGKQTGIIYYVPAGFTSKICPVTGFVNQLYPKYESVSKSQEFFSKFDKICY
NLDKGYFEFSFDYKNFGDKAAKGKWTIASFGSRLINFRNSDKNHNWDTREVYPTKELEKLLKDYSIEY
GHGECIKAAICGESDKKFFAKLTSVLNTILQMRNSKTGTELDYLISPVADVNGNFFDSRQAPKNMPQDA
AANGAYHIGLKGLMLLGRIKNNQEGKKLNLVIKNEEYFEFVQNRNN
Francisella novicida Cpf1 E1006A/D1255A (D917, A1006, and
A1255 are bolded and underlined)
(SEQ ID NO: 266)
MSIYQEFVNKYSLSKTLRFELIPQGKTLENIKARGLILDDEKRAKDYKKAKQIIDKYHQFFIEEILSSVCIS
EDLLQNYSDVYFKLKKSDDDNLQKDFKSAKDTIKKQISEYIKDSEKFKNLFNQNLIDAKKGQESDLILW
LKQSKDNGIELFKANSDITDIDEALEIIKSFKGWTTYFKGFHENRKNVYSSNDIPTSIIYRIVDDNLPKFLE
NKAKYESLKDKAPEAINYEQIKKDLAELLTFDIDYKTSEVNQRVFSLDEVFEIANFNNYLNQSGITKFNT
IIGGKFVNGENTKRKGINEYINLYSQQINDKTLKKYKMSVLFKQILSDTESKSFVIDKLEDDSDVVTTMQ
SFYEQIAAFKTVEEKSIKETLSLLFDDLKAQKLDLSKIYFKNDKSLTDLSQQVFDDYSVIGTAVLEYITQ
QIAPKNLDNPSKKEQELIAKKTEKAKYLSLETIKLALEEFNKHRDIDKQCRFEEILANFAAIPMIFDEIAQ
NKDNLAQISIKYQNQGKKDLLQASAEDDVKAIKDLLDQTNNLLHKLKIFHISQSEDKANILDKDEHFYL
VFEECYFELANIVPLYNKIRNYITQKPYSDEKFKLNFENSTLANGWDKNKEPDNTAILFIKDDKYYLGV
MNKKNNKIFDDKAIKENKGEGYKKIVYKLLPGANKMLPKVFFSAKSIKFYNPSEDILRIRNHSTHTKNG
SPQKGYEKFEFNIEDCRKFIDFYKQSISKHPEWKDFGFRFSDTQRYNSIDEFYREVENQGYKLTFENISES
YIDSVVNQGKLYLFQIYNKDFSAYSKGRPNLHTLYWKALFDERNLQDVVYKLNGEAELFYRKQSIPKK
ITHPAKEAIANKNKDNPKKESVFEYDLIKDKRFTEDKFFFHCPITINFKSSGANKFNDEINLLLKEKAND
VHILSIDRGERHLAYYTLVDGKGNIIKQDTFNIIGNDRMKTNYHDKLAAIEKDRDSARKDWKKINNIKE
MKEGYLSQVVHEIAKLVIEYNAIVVFADLNFGFKRGRFKVEKQVYQKLEKMLIEKLNYLVFKDNEFDK
TGGVLRAYQLTAPFETFKKMGKQTGIIYYVPAGFTSKICPVTGFVNQLYPKYESVSKSQEFFSKFDKICY
NLDKGYFEFSFDYKNFGDKAAKGKWTIASFGSRLINFRNSDKNHNWDTREVYPTKELEKLLKDYSIEY
GHGECIKAAICGESDKKFFAKLTSVLNTILQMRNSKTGTELDYLISPVADVNGNFFDSRQAPKNMPQDA
AANGAYHIGLKGLMLLGRIKNNQEGKKLNLVIKNEEYFEFVQNRNN
Francisella novicida Cpf1 D917A/E1006A/D1255A (A917,
A1006, and A1255 are bolded and underlined)
(SEQ ID NO: 267)
MSIYQEFVNKYSLSKTLRFELIPQGKTLENIKARGLILDDEKRAKDYKKAKQIIDKYHQFFIEEILSSVCIS
EDLLQNYSDVYFKLKKSDDDNLQKDFKSAKDTIKKQISEYIKDSEKFKNLFNQNLIDAKKGQESDLILW
LKQSKDNGIELFKANSDITDIDEALEIIKSFKGWTTYFKGFHENRKNVYSSNDIPTSIIYRIVDDNLPKFLE
NKAKYESLKDKAPEAINYEQIKKDLAELLTFDIDYKTSEVNQRVFSLDEVFEIANFNNYLNQSGITKFNT
IIGGKFVNGENTKRKGINEYINLYSQQINDKTLKKYKMSVLFKQILSDTESKSFVIDKLEDDSDVVTTMQ
SFYEQIAAFKTVEEKSIKETLSLLFDDLKAQKLDLSKIYFKNDKSLTDLSQQVFDDYSVIGTAVLEYITQ
QIAPKNLDNPSKKEQELIAKKTEKAKYLSLETIKLALEEFNKHRDIDKQCRFEEILANFAAIPMIFDEIAQ
NKDNLAQISIKYQNQGKKDLLQASAEDDVKAIKDLLDQTNNLLHKLKIFHISQSEDKANILDKDEHFYL
VFEECYFELANIVPLYNKIRNYITQKPYSDEKFKLNFENSTLANGWDKNKEPDNTAILFIKDDKYYLGV
MNKKNNKIFDDKAIKENKGEGYKKIVYKLLPGANKMLPKVFFSAKSIKFYNPSEDILRIRNHSTHTKNG
SPQKGYEKFEFNIEDCRKFIDFYKQSISKHPEWKDFGFRFSDTQRYNSIDEFYREVENQGYKLTFENISES
YIDSVVNQGKLYLFQIYNKDFSAYSKGRPNLHTLYWKALFDERNLQDVVYKLNGEAELFYRKQSIPKK
ITHPAKEAIANKNKDNPKKESVFEYDLIKDKRFTEDKFFFHCPITINFKSSGANKFNDEINLLLKEKAND
VHILSIARGERHLAYYTLVDGKGNIIKQDTFNIIGNDRMKTNYHDKLAAIEKDRDSARKDWKKINNIKE
MKEGYLSQVVHEIAKLVIEYNAIVVFADLNFGFKRGRFKVEKQVYQKLEKMLIEKLNYLVFKDNEFDK
TGGVLRAYQLTAPFETFKKMGKQTGIIYYVPAGFTSKICPVTGFVNQLYPKYESVSKSQEFFSKFDKICY
NLDKGYFEFSFDYKNFGDKAAKGKWTIASFGSRLINFRNSDKNHNWDTREVYPTKELEKLLKDYSIEY
GHGECIKAAICGESDKKFFAKLTSVLNTILQMRNSKTGTELDYLISPVADVNGNFFDSRQAPKNMPQDA
AANGAYHIGLKGLMLLGRIKNNQEGKKLNLVIKNEEYFEFVQNRNN

In some embodiments, the guide nucleotide sequence-programmable DNA binding protein is a Cpf1 protein from a Acidaminococcus species (AsCpf1). Cpf1 proteins form Acidaminococcus species have been described previously and would be apparent to the skilled artisan. Exemplary Acidaminococcus Cpf1 proteins (AsCpf1) include, without limitation, any of the AsCpf1 proteins provided herein.

Wild-type AsCpf1-Residue R912 is indicated in bold
underlining and residues 661-667 are indicated
in italics and underlining.
(SEQ ID NO: 2473)
TQFEGFTNLYQVSKTLRFELIPQGKTLKHIQEQGFIEEDKARNDHYKELK
PIIDRIYKTYADQCLQLVQLDWENLSAAIDSYRKEKTEETRNALIELQAT
YRNAIHDYFIGRTDNLTDAINKRHAEIYKGLFKAELFNGKVLKQLGTVTT
TEHENALLRSFDKFTTYFSGFYENRKNVFSAEDISTAIPHRIVQDNFPKF
KENCHIFTRLITAVPSLREHFENVKKAIGIFVSTSIEEVFSFPFYNQLLT
QTQIDLYNQLLGGISREAGTEKIKGLNEVLNLAIQKNDETAHIIASLPHR
FIPLFKQILSDRNTLSFILEEFKSDEEVIQSFCKYKTLLRNENVLETAEA
LFNELNSIDLTHIFISHKKLETISSALCDHWDTLRNALYERRISELTGKI
TKSAKEKVQRSLKHEDINLQEIISAAGKELSEAFKQKTSEILSHAHAALD
QPLPTTMLKKQEEKEILKSQLDSLLGLYHLLDWFAVDESNEVDPEFSARL
TGIKLEMEPSLSFYNKARNYATKKPYSVEKFKLNFQMPTLASGWDVNKEK
NNGAILFVKNGLYYLGIMPKQKGRYKALSFEPTEKTSEGFDKMYYDYFPD
AAKMIPKCSTQLKAVTAHFQTHTTPILLSNNFIEPLEITKEIYDLNNPEK
EPKKFQTAYAKKTGDQKGYREALCKWIDFTRDFLSKYTKTTSIDLSSLRP
SSQYKDLGEYYAELNPLLYHISFQRIAEKEIMDAVETGKLYLFQIYNKDF
AKGHHGKPNLHTLYWTGLFSPENLAKTSIKLNGQAELFYRPKSRMKRMAH
RLGEKMLNKKLKDQKTPIPDTLYQELYDYVNHRLSHDLSDEARALLPNVI
TKEVSHEIIKDRRFTSDKFFFHVPITLNYQAANSPSKFNQRVNAYLKEHP
ETPIIGIDRGERNLIYITVIDSTGKILEQRSLNTIQQFDYQKKLDNREKE
RVAARQAWSVVGTIKDLKQGYLSQVIHEIVDLMIHYQAVVVLENLNFGFK
SKRTGIALKAVYQQFEKMLIDKLNCLVLKDYPAEKVGGVLNPYQLTDQFT
SFAKMGTQSGFLFYVPAPYTSKIDPLTGFVDPFVWKTIKNHESRKHFLEG
FDFLHYDVKTGDFILHFKMNRNLSFQRGLPGFMPAWDIVFEKNETQFDAK
GTPFIAGKRIVPVIENHRFTGRYRDLYPANELIALLEEKGIVFRDGSNIL
PKLLENDDSHAIDTMVALIRSVLQMRNSNAATGEDYINSPVRDLNGVCFD
SRFQNPEWPMDADANGAYHIALKGQLLLNHLKESKDLKLQNGISNQDWLA
YIQELRN

AsCpf1(R912A)-Residue A912 is indicated in bold underlining and residues 661-667 are indicated in italics and underlining.

(SEQ ID NO: 2474)
TQFEGFTNLYQVSKTLRFELIPQGKTLKHIQEQGFIEEDKARNDHYKELK
PIIDRIYKTYADQCLQLVQLDWENLSAAIDSYRKEKTEETRNALIELQAT
YRNAIHDYFIGRTDNLTDAINKRHAEIYKGLFKAELFNGKVLKQLGTVTT
TEHENALLRSFDKFTTYFSGFYENRKNVFSAEDISTAIPHRIVQDNFPKF
KENCHIFTRLITAVPSLREHFENVKKAIGIFVSTSIEEVFSFPFYNQLLT
QTQIDLYNQLLGGISREAGTEKIKGLNEVLNLAIQKNDETAHIIASLPHR
FIPLFKQILSDRNTLSFILEEFKSDEEVIQSFCKYKTLLRNENVLETAEA
LFNELNSIDLTHIFISHKKLETISSALCDHWDTLRNALYERRISELTGKI
TKSAKEKVQRSLKHEDINLQEIISAAGKELSEAFKQKTSEILSHAHAALD
QPLPTTMLKKQEEKEILKSQLDSLLGLYHLLDWFAVDESNEVDPEFSARL
TGIKLEMEPSLSFYNKARNYATKKPYSVEKFKLNFQMPTLASGWDVNKEK
NNGAILFVKNGLYYLGIMPKQKGRYKALSFEPTEKTSEGFDKMYYDYFPD
AAKMIPKCSTQLKAVTAHFQTHTTPILLSNNFIEPLEITKEIYDLNNPEK
EPKKFQTAYAKKTGDQKGYREALCKWIDFTRDFLSKYTKTTSIDLSSLRP
SSQYKDLGEYYAELNPLLYHISFQRIAEKEIMDAVETGKLYLFQIYNKDF
AKGHHGKPNLHTLYWTGLFSPENLAKTSIKLNGQAELFYRPKSRMKRMAH
RLGEKMLNKKLKDQKTPIPDTLYQELYDYVNHRLSHDLSDEARALLPNVI
TKEVSHEIIKDRRFTSDKFFFHVPITLNYQAANSPSKFNQRVNAYLKEHP
ETPIIGIDRGEANLIYITVIDSTGKILEQRSLNTIQQFDYQKKLDNREKE
RVAARQAWSVVGTIKDLKQGYLSQVIHEIVDLMIHYQAVVVLENLNFGFK
SKRTGIALKAVYQQFEKMLIDKLNCLVLKDYPAEKVGGVLNPYQLTDQFT
SFAKMGTQSGFLFYVPAPYTSKIDPLTGFVDPFVWKTIKNHESRKHFLEG
FDFLHYDVKTGDFILHFKMNRNLSFQRGLPGFMPAWDIVFEKNETQFDAK
GTPFIAGKRIVPVIENHRFTGRYRDLYPANELIALLEEKGIVFRDGSNIL
PKLLENDDSHAIDTMVALIRSVLQMRNSNAATGEDYINSPVRDLNGVCFD
SRFQNPEWPMDADANGAYHIALKGQLLLNHLKESKDLKLQNGISNQDWLA
YIQELRN

In some embodiments, the nucleic acid programmable DNA binding protein is a Cpf1 protein from a Lachnospiraceae species (LbCpf1). Cpf1 proteins form Lachnospiraceae species have been described previously have been described previously and would be apparent to the skilled artisan. Exemplary Lachnospiraceae Cpf1 proteins (LbCpf1) include, without limitation, any of the LbCpf1 proteins provided herein.

Wild-type LbCpf1
(SEQ ID NO: 2475)
MSKLEKFTNCYSLSKTLRFKAIPVGKTQENIDNKRLLVEDEKRAEDYKGVKKLLDRYYLSFINDVLHSI
KLKNLNNYISLFRKKTRTEKENKELENLEINLRKEIAKAFKGNEGYKSLFKKDIIETILPEFLDDKDEIAL
VNSFNGFTTAFTGFFDNRENMFSEEAKSTSIAFRCINENLTRYISNMDIFEKVDAIFDKHEVQEIKEKILN
SDYDVEDFFEGEFFNFVLTQEGIDVYNAIIGGFVTESGEKIKGLNEYINLYNQKTKQKLPKFKPLYKQVL
SDRESLSFYGEGYTSDEEVLEVFRNTLNKNSEIFSSIKKLEKLFKNFDEYSSAGIFVKNGPAISTISKDIFG
EWNVIRDKWNAEYDDIHLKKKAVVTEKYEDDRRKSFKKIGSFSLEQLQEYADADESVVEKLKEIIIQK
VDEIYKVYGSSEKLFDADFVLEKSLKKNDAVVAIMKDLLDSVKSFENYIKAFFGEGKETNRDESFYGD
FVLAYDILLKVDHIYDAIRNYVTQKPYSKDKFKLYFQNPQFMGGWDKDKETDYRATILRYGSKYYLAI
MDKKYAKCLQKIDKDDVNGNYEKINYKLLPGPNKMLPKVFFSKKWMAYYNPSEDIQKIYKNGTFKK
GDMFNLNDCHKLIDFFKDSISRYPKWSNAYDFNFSETEKYKDIAGFYREVEEQGYKVSFESASKKEVD
KLVEEGKLYMFQIYNKDFSDKSHGTPNLHTMYFKLLFDENNHGQIRLSGGAELFMRRASLKKEELVVH
PANSPIANKNPDNPKKTTTLSYDVYKDKRFSEDQYELHIPIAINKCPKNIFKINTEVRVLLKHDDNPYVI
GIDRGERNLLYIVVVDGKGNIVEQYSLNEIINNFNGIRIKTDYHSLLDKKEKERFEARQNWTSIENIKELK
AGYISQVVHKICELVEKYDAVIALEDLNSGFKNSRVKVEKQVYQKFEKMLIDKLNYMVDKKSNPCAT
GGALKGYQITNKFESFKSMSTQNGFIFYIPAWLTSKIDPSTGFVNLLKTKYTSIADSKKFISSFDRIMYVP
EEDLFEFALDYKNFSRTDADYIKKWKLYSYGNRIRIFRNPKKNNVFDWEEVCLTSAYKELFNKYGINY
QQGDIRALLCEQSDKAFYSSFMALMSLMLQMRNSITGRTDVDFLISPVKNSDGIFYDSRNYEAQENAIL
PKNADANGAYNIARKVLWAIGQFKKAEDEKLDKVKIAISNKEWLEYAQTSVKH
LbCpf1 (R836A)
(SEQ ID NO: 2476)
MSKLEKFTNCYSLSKTLRFKAIPVGKTQENIDNKRLLVEDEKRAEDYKGVKKLLDRYYLSFINDVLHSI
KLKNLNNYISLFRKKTRTEKENKELENLEINLRKEIAKAFKGNEGYKSLFKKDIIETILPEFLDDKDEIAL
VNSFNGFTTAFTGFFDNRENMFSEEAKSTSIAFRCINENLTRYISNMDIFEKVDAIFDKHEVQEIKEKILN
SDYDVEDFFEGEFFNFVLTQEGIDVYNAIIGGFVTESGEKIKGLNEYINLYNQKTKQKLPKFKPLYKQVL
SDRESLSFYGEGYTSDEEVLEVFRNTLNKNSEIFSSIKKLEKLFKNFDEYSSAGIFVKNGPAISTISKDIFG
EWNVIRDKWNAEYDDIHLKKKAVVTEKYEDDRRKSFKKIGSFSLEQLQEYADADLSVVEKLKEIIIQK
VDEIYKVYGSSEKLFDADFVLEKSLKKNDAVVAIMKDLLDSVKSFENYIKAFFGEGKETNRDESFYGD
FVLAYDILLKVDHIYDAIRNYVTQKPYSKDKFKLYFQNPQFMGGWDKDKETDYRATILRYGSKYYLAI
MDKKYAKCLQKIDKDDVNGNYEKINYKLLPGPNKMLPKVFFSKKWMAYYNPSEDIQKIYKNGTFKK
GDMFNLNDCHKLIDFFKDSISRYPKWSNAYDFNFSETEKYKDIAGFYREVEEQGYKVSFESASKKEVD
KLVEEGKLYMFQIYNKDFSDKSHGTPNLHTMYFKLLFDENNHGQIRLSGGAELFMRRASLKKEELVVH
PANSPIANKNPDNPKKTTTLSYDVYKDKRFSEDQYELHIPIAINKCPKNIFKINTEVRVLLKHDDNPYVI
GIDRGEANLLYIVVVDGKGNIVEQYSLNEIINNFNGIRIKTDYHSLLDKKEKERFEARQNWTSIENIKEL
KAGYISQVVHKICELVEKYDAVIALEDENSGFKNSRVKVEKQVYQKFEKMLIDKLNYMVDKKSNPCA
TGGALKGYQITNKFESFKSMSTQNGFIFYIPAWLTSKIDPSTGFVNLLKTKYTSIADSKKFISSFDRIMYV
PEEDLFEFALDYKNFSRTDADYIKKWKLYSYGNRIRIFRNPKKNNVFDWEEVCLTSAYKELFNKYGIN
YQQGDIRALLCEQSDKAFYSSFMALMSLMLQMRNSITGRTDVDFLISPVKNSDGIFYDSRNYEAQENAI
LPKNADANGAYNIARKVLWAIGQFKKAEDEKLDKVKIAISNKEWLEYAQTSVKH
LbCpf1 (R1138A)
(SEQ ID NO: 2477)
MSKLEKFTNCYSLSKTERFKAIPVGKTQENIDNKRLLVEDEKRAEDYKGVKKLLDRYYLSFINDVLHSI
KLKNLNNYISLFRKKTRTEKENKELENLEINLRKEIAKAFKGNEGYKSLFKKDIIETILPEFLDDKDEIAL
VNSFNGFTTAFTGFFDNRENMFSEEAKSTSIAFRCINENLTRYISNMDIFEKVDAIFDKHEVQEIKEKILN
SDYDVEDFFEGEFFNFVLTQEGIDVYNAIIGGFVTESGEKIKGLNEYINLYNQKTKQKLPKFKPLYKQVL
SDRESLSFYGEGYTSDEEVLEVFRNTLNKNSEIFSSIKKLEKLFKNFDEYSSAGIFVKNGPAISTISKDIFG
EWNVIRDKWNAEYDDIHLKKKAVVTEKYEDDRRKSFKKIGSFSLEQLQEYADADLSVVEKLKEIIIQK
VDEIYKVYGSSEKLFDADFVLEKSLKKNDAVVAIMKDLLDSVKSFENYIKAFFGEGKETNRDESFYGD
FVLAYDILLKVDHIYDAIRNYVTQKPYSKDKFKLYFQNPQFMGGWDKDKETDYRATILRYGSKYYLAI
MDKKYAKCLQKIDKDDVNGNYEKINYKLLPGPNKMLPKVFFSKKWMAYYNPSEDIQKIYKNGTFKK
GDMFNLNDCHKLIDFFKDSISRYPKWSNAYDFNFSETEKYKDIAGFYREVEEQGYKVSFESASKKEVD
KLVEEGKLYMFQIYNKDFSDKSHGTPNLHTMYFKLLFDENNHGQIRLSGGAELFMRRASLKKEELVVH
PANSPIANKNPDNPKKTTTLSYDVYKDKRFSEDQYELHIPIAINKCPKNIFKINTEVRVLLKHDDNPYVI
GIDRGERNLLYIVVVDGKGNIVEQYSLNEIINNFNGIRIKTDYHSLLDKKEKERFEARQNWTSIENIKELK
AGYISQVVHKICELVEKYDAVIALEDLNSGFKNSRVKVEKQVYQKFEKMLIDKLNYMVDKKSNPCAT
GGALKGYQITNKFESFKSMSTQNGFIFYIPAWLTSKIDPSTGFVNLLKTKYTSIADSKKFISSFDRIMYVP
EEDLFEFALDYKNFSRTDADYIKKWKLYSYGNRIRIFRNPKKNNVFDWEEVCLTSAYKELFNKYGINY
QQGDIRALLCEQSDKAFYSSFMALMSLMLQMANSITGRTDVDFLISPVKNSDGIFYDSRNYEAQENAIL
PKNADANGAYNIARKVLWAIGQFKKAEDEKLDKVKIAISNKEWLEYAQTSVKH

In some embodiments, the Cpf1 protein is a crippled Cpf1 protein. As used herein a “crippled Cpf1” protein is a Cpf1 protein having diminished nuclease activity as compared to a wild-type Cpf1 protein. In some embodiments, the crippled Cpf1 protein preferentially cuts the target strand more efficiently than the non-target strand. For example, the Cpf1 protein preferentially cuts the strand of a duplexed nucleic acid molecule in which a nucleotide to be edited resides. In some embodiments, the crippled Cpf1 protein preferentially cuts the non-target strand more efficiently than the target strand. For example, the Cpf1 protein preferentially cuts the strand of a duplexed nucleic acid molecule in which a nucleotide to be edited does not reside. In some embodiments, the crippled Cpf1 protein preferentially cuts the target strand at least 5% more efficiently than it cuts the non-target strand. In some embodiments, the crippled Cpf1 protein preferentially cuts the target strand at least 5%, 10%, 15%, 20%, 25%, 30%, 35%, 40%, 50%, 60%, 70%, 80%, 90%, or at least 100% more efficiently than it cuts the non-target strand.

In some embodiments, a crippled Cpf1 protein is a non-naturally occurring Cpf1 protein. In some embodiments, the crippled Cpf1 protein comprises one or more mutations relative to a wild-type Cpf1 protein. In some embodiments, the crippled Cpf1 protein comprises 1, 2, 3, 4, 5, 6, 7, 8, 9, 10, 11, 12, 13, 14, 15, 16, 17, 18, 19, or 20 mutations relative to a wild-type Cpf1 protein. In some embodiments, the crippled Cpf1 protein comprises an R836A mutation as set forth in SEQ ID NO: 763, or in a corresponding amino acid in another Cpf1 protein. It should be appreciated that a Cpf1 comprising a homologous residue (e.g., a corresponding amino acid) to R836A of SEQ ID NO: 763 could also be mutated to achieve similar results. In some embodiments, the crippled Cpf1 protein comprises a R1138A mutation as set forth in SEQ ID NO: 763, or in a corresponding amino acid in another Cpf1 protein. In some embodiments, the crippled Cpf1 protein comprises an R912A mutation as set forth in SEQ ID NO: 762, or in a corresponding amino acid in another Cpf1 protein. Without wishing to be bound by any particular theory, residue R838 of SEQ ID NO: 763 (LbCpf1) and residue R912 of SEQ ID NO: 762 (AsCpf1) are examples of corresponding (e.g., homologous) residues. For example, a portion of the alignment between SEQ ID NO: 762 and 763 shows that R912 and R838 are corresponding residues.

In some embodiments, any of the Cpf1 proteins provided herein comprises one or more amino acid deletions. In some embodiments, any of the Cpf1 proteins provided herein comprises 1, 2, 3, 4, 5, 6, 7, 8, 9, 10, 11, 12, 13, 14, 15, 16, 17, 18, 19, or 20 amino acid deletions. Without wishing to be bound by any particular theory, there is a helical region in Cpf1, which includes residues 661-667 of AsCpf1 (SEQ ID NO: 762), that may obstruct the function of a deaminase (e.g., APOBEC) that is fused to the Cpf1. This region comprises the amino acid sequence KKTGDQK. Accordingly, aspects of the disclosure provide Cpf1 proteins comprising mutations (e.g., deletions) that disrupt this helical region in Cpf1. In some embodiments, the Cpf1 protein comprises one or more deletions of the following residues in SEQ ID NO: 762, or one or more corresponding deletions in another Cpf1 protein: K661, K662, T663, G664, D665, Q666, and K667. In some embodiments, the Cpf1 protein comprises a T663 and a D665 deletion in SEQ ID NO: 762, or corresponding deletions in another Cpf1 protein. In some embodiments, the Cpf1 protein comprises a K662, T663, D665, and Q666 deletion in SEQ ID NO: 762, or corresponding deletions in another Cpf1 protein. In some embodiments, the Cpf1 protein comprises a K661, K662, T663, D665, Q666 and K667 deletion in SEQ ID NO: 762, or corresponding deletions in another Cpf1 protein.

AsCpf1 (deleted T663 and D665)
(SEQ ID NO: 2478)
TQFEGFTNLYQVSKTLRFELIPQGKTLKHIQEQGFIEEDKARNDHYKELKPIIDRIYKTYADQCLQLVQL
DWENLSAAIDSYRKEKTEETRNALIELQATYRNAIHDYFIGRTDNLTDAINKRHAEIYKGLFKAELFNG
KVLKQLGTVTTTEHENALLRSFDKFTTYFSGFYENRKNVFSAEDISTAIPHRIVQDNFPKFKENCHIFTRL
ITAVPSLREHFENVKKAIGIFVSTSIEEVFSFPFYNQLLTQTQIDLYNQLLGGISREAGTEKIKGLNEVLNL
AIQKNDETAHIIASLPHRFIPLFKQILSDRNTLSFILEEFKSDEEVIQSFCKYKTLLRNENVLETAEALFNEL
NSIDLTHIFISHKKLETISSALCDHWDTLRNALYERRISELTGKITKSAKEKVQRSLKHEDINLQEIISAAG
KELSEAFKQKTSEILSHAHAALDQPLPTTMLKKQEEKEILKSQLDSLLGLYHLLDWFAVDESNEVDPEF
SARLTGIKLEMEPSLSFYNKARNYATKKPYSVEKFKLNFQMPTLASGWDVNKEKNNGAILFVKNGLY
YLGIMPKQKGRYKALSFEPTEKTSEGFDKMYYDYFPDAAKMIPKCSTQLKAVTAHFQTHTTPILLSNNF
IEPLEITKEIYDLNNPEKEPKKFQTAYAKKGQKGYREALCKWIDFTRDFLSKYTKTTSIDLSSLRPSSQY
KDLGEYYAELNPLLYHISFQRIAEKEIMDAVETGKLYLFQIYNKDFAKGHHGKPNLHTLYWTGLFSPEN
LAKTSIKLNGQAELFYRPKSRMKRMAHRLGEKMLNKKLKDQKTPIPDTLYQELYDYVNHRLSHDLSD
EARALLPNVITKEVSHEIIKDRRFTSDKFFFHVPITLNYQAANSPSKFNQRVNAYLKEHPETPIIGIDRGER
NLIYITVIDSTGKILEQRSLNTIQQFDYQKKLDNREKERVAARQAWSVVGTIKDLKQGYLSQVIHEIVDL
MIHYQAVVVLENLNFGFKSKRTGIALKAVYQQFEKMLIDKLNCLVLKDYPAEKVGGVLNPYQLTDQF
TSFAKMGTQSGFLFYVPAPYTSKIDPLTGFVDPFVWKTIKNHESRKHFLEGFDFLHYDVKTGDFILHFK
MNRNLSFQRGLPGFMPAWDIVFEKNETQFDAKGTPFIAGKRIVPVIENHRFTGRYRDLYPANELIALLE
EKGIVFRDGSNILPKLLENDDSHAIDTMVALIRSVLQMRNSNAATGEDYINSPVRDLNGVCFDSRFQNP
EWPMDADANGAYHIALKGQLLLNHLKESKDLKLQNGISNQDWLAYIQELRN
AsCpf1 (deleted K662, T663, D665, and Q666)
(SEQ ID NO: 2479)
TQFEGFTNLYQVSKTLRFELIPQGKTLKHIQEQGFIEEDKARNDHYKELKPIIDRIYKTYADQCLQLVQL
DWENLSAAIDSYRKEKTEETRNALIELQATYRNAIHDYFIGRTDNLTDAINKRHAEIYKGLFKAELFNG
KVLKQLGTVTTTEHENALLRSFDKFTTYFSGFYENRKNVFSAEDISTAIPHRIVQDNFPKFKENCHIFTRL
ITAVPSLREHFENVKKAIGIFVSTSIEEVFSFPFYNQLLTQTQIDLYNQLLGGISREAGTEKIKGLNEVLNL
AIQKNDETAHIIASLPHRFIPLFKQILSDRNTLSFILEEFKSDEEVIQSFCKYKTLLRNENVLETAEALFNEL
NSIDLTHIFISHKKLETISSALCDHWDTLRNALYERRISELTGKITKSAKEKVQRSLKHEDINLQEIISAAG
KELSEAFKQKTSEILSHAHAALDQPLPTTMLKKQEEKEILKSQLDSLLGLYHLLDWFAVDESNEVDPEF
SARLTGIKLEMEPSLSFYNKARNYATKKPYSVEKFKLNFQMPTLASGWDVNKEKNNGAILFVKNGLY
YLGIMPKQKGRYKALSFEPTEKTSEGFDKMYYDYFPDAAKMIPKCSTQLKAVTAHFQTHTTPILLSNNF
IEPLEITKEIYDLNNPEKEPKKFQTAYAKGKGYREALCKWIDFTRDFLSKYTKTTSIDLSSLRPSSQYKDL
GEYYAELNPLLYHISFQRIAEKEIMDAVETGKLYLFQIYNKDFAKGHHGKPNLHTLYWTGLFSPENLAK
TSIKLNGQAELFYRPKSRMKRMAHRLGEKMLNKKLKDQKTPIPDTLYQELYDYVNHRLSHDLSDEAR
ALLPNVITKEVSHEIIKDRRFTSDKFFFHVPITLNYQAANSPSKFNQRVNAYLKEHPETPIIGIDRGERNLI
YITVIDSTGKILEQRSLNTIQQFDYQKKLDNREKERVAARQAWSVVGTIKDLKQGYLSQVIHEIVDLMI
HYQAVVVLENLNFGFKSKRTGIAEKAVYQQFEKMLIDKLNCLVLKDYPAEKVGGVLNPYQLTDQFTS
FAKMGTQSGFLFYVPAPYTSKIDPLTGFVDPFVWKTIKNHESRKHFLEGFDFLHYDVKTGDFILHFKMN
RNLSFQRGLPGFMPAWDIVFEKNETQFDAKGTPFIAGKRIVPVIENHRFTGRYRDLYPANELIALLEEKG
IVFRDGSNILPKLLENDDSHAIDTMVALIRSVLQMRNSNAATGEDYINSPVRDLNGVCFDSRFQNPEWP
MDADANGAYHIALKGQLLLNHLKESKDLKLQNGISNQDWLAYIQELRN
AsCpf1 (deleted K661, K662, T663, D665, Q666, and K667)
(SEQ ID NO: 2480)
TQFEGFTNLYQVSKTLRFELIPQGKTLKHIQEQGFIEEDKARNDHYKELKPIIDRIYKTYADQCLQLVQL
DWENLSAAIDSYRKEKTEETRNALIEEQATYRNAIHDYFIGRTDNLTDAINKRHAEIYKGLFKAELFNG
KVLKQLGTVTTTEHENALLRSFDKFTTYFSGFYENRKNVFSAEDISTAIPHRIVQDNFPKFKENCHIFTRL
ITAVPSLREHFENVKKAIGIFVSTSIEEVFSFPFYNQLLTQTQIDLYNQLLGGISREAGTEKIKGLNEVLNL
AIQKNDETAHIIASLPHRFIPLFKQILSDRNTLSFILEEFKSDEEVIQSFCKYKTLLRNENVLETAEALFNEL
NSIDLTHIFISHKKLETISSALCDHWDTLRNALYERRISELTGKITKSAKEKVQRSLKHEDINLQEIISAAG
KELSEAFKQKTSEILSHAHAALDQPLPTTMLKKQEEKEILKSQLDSLLGLYHLLDWFAVDESNEVDPEF
SARLTGIKLEMEPSLSFYNKARNYATKKPYSVEKFKLNFQMPTLASGWDVNKEKNNGAILFVKNGLY
YLGIMPKQKGRYKALSFEPTEKTSEGFDKMYYDYFPDAAKMIPKCSTQLKAVTAHFQTHTTPILLSNNF
IEPLEITKEIYDLNNPEKEPKKFQTAYAGGYREALCKWIDFTRDFLSKYTKTTSIDLSSLRPSSQYKDLGE
YYAELNPLLYHISFQRIAEKEIMDAVETGKLYLFQIYNKDFAKGHHGKPNLHTLYWTGLFSPENLAKTS
IKLNGQAELFYRPKSRMKRMAHRLGEKMLNKKLKDQKTPIPDTLYQELYDYVNHRLSHDLSDEARAL
LPNVITKEVSHEIIKDRRFTSDKFFFHVPITLNYQAANSPSKFNQRVNAYLKEHPETPIIGIDRGERNLIYIT
VIDSTGKILEQRSLNTIQQFDYQKKLDNREKERVAARQAWSVVGTIKDLKQGYLSQVIHEIVDLMIHYQ
AVVVLENLNFGFKSKRTGIAEKAVYQQFEKMLIDKLNCLVLKDYPAEKVGGVLNPYQLTDQFTSFAK
MGTQSGFLFYVPAPYTSKIDPLTGFVDPFVWKTIKNHESRKHFLEGFDFLHYDVKTGDFILHFKMNRNL
SFQRGLPGFMPAWDIVFEKNETQFDAKGTPFIAGKRIVPVIENHRFTGRYRDLYPANELIALLEEKGIVF
RDGSNILPKLLENDDSHAIDTMVALIRSVLQMRNSNAATGEDYINSPVRDLNGVCFDSRFQNPEWPMD
ADANGAYHIALKGQLLLNHLKESKDLKLQNGISNQDWLAYIQELRN

In some embodiments, the guide nucleotide sequence-programmable DNA-binding protein domain of the present disclosure has no requirements for a PAM sequence. One example of such a guide nucleotide sequence-programmable DNA-binding protein may be an Argonaute protein from Natronobacterium gregoryi (NgAgo). NgAgo is a ssDNA-guided endonuclease. NgAgo binds 5′ phosphorylated ssDNA of ˜24 nucleotides (gDNA) to guide it to its target site and will make DNA double-strand breaks at gDNA site. In contrast to Cas9, the NgAgo-gDNA system does not require a protospacer-adjacent motif (PAM). Using a nuclease inactive NgAgo (dNgAgo) can greatly expand the bases or codons that may be targeted. The characterization and use of NgAgo have been described in Gao et al., Nat Biotechnol. Epub 2016 May 2. PubMed PMID: 27136078; Swarts et al., Nature. 507(7491) (2014):258-61; and Swarts et al., Nucleic Acids Res. 43(10) (2015):5120-9, each of which are incorporated herein by reference. The sequence of Natronobacterium gregoryi Argonaute is provided in SEQ ID NO: 270.

Wild type Natronobacterium gregoryi Argonaute
(SEQ ID NO: 270)
MTVIDLDSTTTADELTSGHTYDISVTLTGVYDNTDEQHPRMSLAFEQDNG
ERRYITLWKNTTPKDVFTYDYATGSTYIFTNIDYEVKDGYENLTATYQTT
VENATAQEVGTTDEDETFAGGEPLDHHLDDALNETPDDAETESDSGHVMT
SFASRDQLPEWTLHTYTLTATDGAKTDTEYARRTLAYTVRQELYTDHDAA
PVATDGLMLLTPEPLGETPLDLDCGVRVEADETRTLDYTTAKDRLLAREL
VEEGLKRSLWDDYLVRGIDEVLSKEPVLTCDEFDLHERYDLSVEVGHSGR
AYLHINFRHRFVPKLTLADIDDDNIYPGLRVKTTYRPRRGHIVWGLRDEC
ATDSLNTLGNQSVVAYHRNNQTPINTDLLDAIEAADRRVVETRRQGHGDD
AVSFPQELLAVEPNTHQIKQFASDGFHQQARSKTRLSASRCSEKAQAFAE
RLDPVRLNGSTVEFSSEFFTGNNEQQLRLLYENGESVLTFRDGARGAHPD
ETFSKGIVNPPESFEVAVVLPEQQADTCKAQWDTMADLLNQAGAPPTRSE
TVQYDAFSSPESISLNVAGAIDPSEVDAAFVVLPPDQEGFADLASPTETY
DELKKALANMGIYSQMAYFDRFRDAKIFYTRNVALGLLAAAGGVAFTTEH
AMPGDADMFIGIDVSRSYPEDGASGQINIAATATAVYKDGTILGHSSTRP
QLGEKLQSTDVRDIMKNAILGYQQVTGESPTHIVIHRDGFMNEDLDPATE
FLNEQGVEYDIVEIRKQPQTRLLAVSDVQYDTPVKSIAAINQNEPRATVA
TFGAPEYLATRDGGGLPRPIQIERVAGETDIETLTRQVYLLSQSHIQVHN
STARLPITTAYADQASTHATKGYLVQTGAFESNVGFL

Also provided herein are Cas9 variants that have relaxed PAM requirements (PAMless Cas9). PAMless Cas9 exhibits an increased activity on a target sequence that does not include a canonical PAM (e.g., NGG) at its 3′-end as compared to Streptococcus pyogenes Cas9 as provided by SEQ ID NO: 1, e.g., increased activity by at least 5-fold, at least 10-fold, at least 50-fold, at least 100-fold, at least 500-fold, at least 1,000-fold, at least 5,000-fold, at least 10,000-fold, at least 50,000-fold, at least 100,000-fold, at least 500,000-fold, or at least 1,000,000-fold. Such Cas9 variants that have relaxed PAM requirements are described in U.S. Provisional Applications, U.S. Ser. No. 62/245,828, 62/279,346, 62/311,763, 62/322,178, and 62/357,332, each of which is incorporated herein by reference. In some embodiments, the dCas9 or Cas9 nickase useful in the present disclosure may further comprise mutations that relax the PAM requirements, e.g., mutations that correspond to A262T, K294R, S4091, E480K, E543D, M694I, or E1219V in SEQ ID NO: 1.

Other non-limiting, exemplary Cas9 variants (including dCas9, Cas9 nickase, and Cas9 variants with alternative PAM requirements) suitable for use in the nucleobase editors useful in the present disclosure and their respective sequences are provided below.

VRER-nCas9 (D10A/D1135V/G1218R/R1335E/T1337R) S. pyogenes Cas9 Nickase
(SEQ ID NO: 2426)
MDKKYSIGLAIGTNSVGWAVITDEYKVPSKKFKVLGNTDRHSIKKNLIGALLFDSGETAEATRLKRTAR
RRYTRRKNRICYLQEIFSNEMAKVDDSFFHRLEESFLVEEDKKHERHPIFGNIVDEVAYHEKYPTIYHLR
KKLVDSTDKADLRLIYLALAHMIKFRGHFLIEGDLNPDNSDVDKLFIQLVQTYNQLFEENPINASGVDA
KAILSARLSKSRRLENLIAQLPGEKKNGLFGNLIALSLGLTPNFKSNFDLAEDAKLQLSKDTYDDDLDN
LLAQIGDQYADLFLAAKNLSDAILLSDILRVNTEITKAPLSASMIKRYDEHHQDLTLLKALVRQQLPEK
YKEIFFDQSKNGYAGYIDGGASQLEFYKFIKPILEKMDGTEELLVKLNREDLLRKQRTFDNGSIPHQIHL
GELHAILRRQEDFYPFLKDNREKIEKILTFRIPYYVGPLARGNSRFAWMTRKSEETITPWNFEEVVDKGA
SAQSFIERMTNFDKNLPNEKVLPKHSLLYEYFTVYNELTKVKYVTEGMRKPAFLSGEQKKAIVDLLFK
TNRKVTVKQLKEDYFKKIECFDSVEISGVEDRFNASLGTYHDLLKIIKDKDFLDNEENEDILEDIVLTLT
LFEDREMIEERLKTYAHLFDDKVMKQLKRRRYTGWGRLSRKLINGIRDKQSGKTILDFLKSDGFANRN
FMQLIHDDSLTFKEDIQKAQVSGQGDSLHEHIANLAGSPAIKKGILQTVKVVDELVKVMGRHKPENIVI
EMARENQTTQKGQKNSRERMKRIEEGIKELGSQILKEHPVENTQLQNEKLYLYYLQNGRDMYVDQEL
DINRLSDYDVDHIVPQSFLKDDSIDNKVLTRSDKNRGKSDNVPSEEVVKKMKNYWRQLLNAKLITQRK
FDNLTKAERGGLSELDKAGFIKRQLVETRQITKHVAQILDSRMNTKYDENDKLIREVKVITLKSKLVSD
FRKDFQFYKVREINNYHHAHDAYLNAVVGTALIKKYPKLESEFVYGDYKVYDVRKMIAKSEQEIGKA
TAKYFFYSNIMNFFKTEITLANGEIRKRPLIETNGETGEIVWDKGRDFATVRKVLSMPQVNIVKKTEVQ
TGGFSKESILPKRNSDKLIARKKDWDPKKYGGFVSPTVAYSVLVVAKVEKGKSKKLKSVKELLGITIME
RSSFEKNPIDFLEAKGYKEVKKDLIIKLPKYSLFELENGRKRMLASARELQKGNELALPSKYVNFLYLA
SHYEKLKGSPEDNEQKQLFVEQHKHYLDEIIEQISEFSKRVILADANLDKVLSAYNKHRDKPIREQAENI
IHLFTLTNLGAPAAFKYFDTTIDRKEYRSTKEVLDATLIHQSITGLYETRIDLSQLGGD
(single underline: HNH domain; double underline: RuvC domain)
VQR-nCas9 (D10A/D1135V/R1335Q/T1337R) S. pyogenes Cas9 Nickase
(SEQ ID NO: 2427)
MDKKYSIGLAIGTNSVGWAVITDEYKVPSKKFKVLGNTDRHSIKKNLIGALLFDSGETAEATRLKRTAR
RRYTRRKNRICYLQEIFSNEMAKVDDSFFHRLEESFLVEEDKKHERHPIFGNIVDEVAYHEKYPTIYHLR
KKLVDSTDKADLRLIYLALAHMIKFRGHFLIEGDLNPDNSDVDKLFIQLVQTYNQLFEENPINASGVDA
KAILSARLSKSRRLENLIAQLPGEKKNGLFGNLIALSLGLTPNFKSNFDLAEDAKLQLSKDTYDDDLDN
LLAQIGDQYADLFLAAKNLSDAILLSDILRVNTEITKAPLSASMIKRYDEHHQDLTLLKALVRQQLPEK
YKEIFFDQSKNGYAGYIDGGASQLEFYKFIKPILEKMDGTEELLVKLNREDLLRKQRTFDNGSIPHQIHL
GELHAILRRQEDFYPFLKDNREKIEKILTFRIPYYVGPLARGNSRFAWMTRKSEETITPWNFEEVVDKGA
SAQSFIERMTNFDKNLPNEKVLPKHSLLYEYFTVYNELTKVKYVTEGMRKPAFLSGEQKKAIVDLLFK
TNRKVTVKQLKEDYFKKIECFDSVEISGVEDRFNASLGTYHDLLKIIKDKDFLDNEENEDILEDIVLTLT
LFEDREMIEERLKTYAHLFDDKVMKQLKRRRYTGWGRLSRKLINGIRDKQSGKTILDFLKSDGFANRN
FMQLIHDDSLTFKEDIQKAQVSGQGDSLHEHIANLAGSPAIKKGILQTVKVVDELVKVMGRHKPENIVI
EMARENQTTQKGQKNSRERMKRIEEGIKELGSQILKEHPVENTQLQNEKLYLYYLQNGRDMYVDQEL
DINRLSDYDVDHIVPQSFLKDDSIDNKVLTRSDKNRGKSDNVPSEEVVKKMKNYWRQLLNAKLITQRK
FDNLTKAERGGLSELDKAGFIKRQLVETRQITKHVAQILDSRMNTKYDENDKLIREVKVITLKSKLVSD
FRKDFQFYKVREINNYHHAHDAYLNAVVGTALIKKYPKLESEFVYGDYKVYDVRKMIAKSEQEIGKA
TAKYFFYSNIMNFFKTEITLANGEIRKRPLIETNGETGEIVWDKGRDFATVRKVLSMPQVNIVKKTEVQ
TGGFSKESILPKRNSDKLIARKKDWDPKKYGGFVSPTVAYSVLVVAKVEKGKSKKLKSVKELLGITIME
RSSFEKNPIDFLEAKGYKEVKKDLIIKLPKYSLFELENGRKRMLASAGELQKGNELALPSKYVNFLYLA
SHYEKLKGSPEDNEQKQLFVEQHKHYLDEIIEQISEFSKRVILADANLDKVLSAYNKHRDKPIREQAENI
IHLFTLTNLGAPAAFKYFDTTIDRKQYRSTKEVLDATLIHQSITGLYETRIDLSQLGGD
(single underline: HNH domain; double underline: RuvC domain)
EQR-nCas9 (D10A/D1135E/R1335Q/T1337R) S. pyogenes Cas9 Nickase
(SEQ ID NO: 2428)
MDKKYSIGLAIGTNSVGWAVITDEYKVPSKKFKVLGNTDRHSIKKNLIGALLFDSGETAEATRLKRTAR
RRYTRRKNRICYLQEIFSNEMAKVDDSFFHRLEESFLVEEDKKHERHPIFGNIVDEVAYHEKYPTIYHLR
KKLVDSTDKADLRLIYLALAHMIKFRGHFLIEGDLNPDNSDVDKLFIQLVQTYNQLFEENPINASGVDA
KAILSARLSKSRRLENLIAQLPGEKKNGLFGNLIALSLGLTPNFKSNFDLAEDAKLQLSKDTYDDDLDN
LLAQIGDQYADLFLAAKNLSDAILLSDILRVNTEITKAPLSASMIKRYDEHHQDLTLLKALVRQQLPEK
YKEIFFDQSKNGYAGYIDGGASQLEFYKFIKPILEKMDGTEELLVKLNREDLLRKQRTFDNGSIPHQIHL
GELHAILRRQEDFYPFLKDNREKIEKILTFRIPYYVGPLARGNSRFAWMTRKSEETITPWNFEEVVDKGA
SAQSFIERMTNFDKNLPNEKVLPKHSLLYEYFTVYNELTKVKYVTEGMRKPAFLSGEQKKAIVDLLFK
TNRKVTVKQLKEDYFKKIECFDSVEISGVEDRFNASLGTYHDLLKIIKDKDFLDNEENEDILEDIVLTLT
LFEDREMIEERLKTYAHLFDDKVMKQLKRRRYTGWGRLSRKLINGIRDKQSGKTILDFLKSDGFANRN
FMQLIHDDSLTFKEDIQKAQVSGQGDSLHEHIANLAGSPAIKKGILQTVKVVDELVKVMGRHKPENIVI
EMARENQTTQKGQKNSRERMKRIEEGIKELGSQILKEHPVENTQLQNEKLYLYYLQNGRDMYVDQEL
DINRLSDYDVDHIVPQSFLKDDSIDNKVLTRSDKNRGKSDNVPSEEVVKKMKNYWRQLLNAKLITQRK
FDNLTKAERGGLSELDKAGFIKRQLVETRQITKHVAQILDSRMNTKYDENDKLIREVKVITLKSKLVSD
FRKDFQFYKVREINNYHHAHDAYLNAVVGTALIKKYPKLESEFVYGDYKVYDVRKMIAKSEQEIGKA
TAKYFFYSNIMNFFKTEITLANGEIRKRPLIETNGETGEIVWDKGRDFATVRKVLSMPQVNIVKKTEVQ
TGGFSKESILPKRNSDKLIARKKDWDPKKYGGFESPTVAYSVLVVAKVEKGKSKKLKSVKELLGITIME
RSSFEKNPIDFLEAKGYKEVKKDLIIKLPKYSLFELENGRKRMLASAGELQKGNELALPSKYVNFLYLA
SHYEKLKGSPEDNEQKQLFVEQHKHYLDEIIEQISEFSKRVILADANLDKVLSAYNKHRDKPIREQAENI
IHLFTLTNLGAPAAFKYFDTTIDRKQYRSTKEVLDATLIHQSITGLYETRIDLSQLGGD
(single underline: HNH domain; double underline: RuvC domain)
KKH-nCas9 (D10A/E782K/N968K/R1015H) S. aureus Cas9 Nickase
(SEQ ID NO: 268)
MKRNYILGLAIGITSVGYGIIDYETRDVIDAGVRLFKEANVENNEGRRSKRGARRLKRRRRHRIQRVKK
LLFDYNLLTDHSELSGINPYEARVKGLSQKLSEEEFSAALLHLAKRRGVHNVNEVEEDTGNELSTKEQI
SRNSKALEEKYVAELQLERLKKDGEVRGSINRFKTSDYVKEAKQLLKVQKAYHQLDQSFIDTYIDLLE
TRRTYYEGPGEGSPFGWKDIKEWYEMLMGHCTYFPEELRSVKYAYNADLYNALNDLNNLVITRDENE
KLEYYEKFQIIENVFKQKKKPTLKQIAKEILVNEEDIKGYRVTSTGKPEFTNLKVYHDIKDITARKEIIEN
AELLDQIAKILTIYQSSEDIQEELTNLNSELTQEEIEQISNLKGYTGTHNLSLKAINLILDELWHTNDNQIA
IFNRLKLVPKKVDLSQQKEIPTTLVDDFILSPVVKRSFIQSIKVINAIIKKYGLPNDIIIELAREKNSKDAQK
MINEMQKRNRQTNERIEEIIRTTGKENAKYLIEKIKLHDMQEGKCLYSLEAIPLEDLLNNPFNYEVDHIIP
RSVSFDNSFNNKVLVKQEENSKKGNRTPFQYLSSSDSKISYETFKKHILNLAKGKGRISKTKKEYLLEER
DINRFSVQKDFINRNLVDTRYATRGLMNLLRSYFRVNNLDVKVKSINGGFTSFLRRKWKFKKERNKGY
KHHAEDALIIANADFIFKEWKKLDKAKKVMENQMFEEKQAESMPEIETEQEYKEIFITPHQIKHIKDFK
DYKYSHRVDKKPNRKLINDTLYSTRKDDKGNTLIVNNLNGLYDKDNDKLKKLINKSPEKLLMYHHDP
QTYQKLKLIMEQYGDEKNPLYKYYEETGNYLTKYSKKDNGPVIKKIKYYGNKLNAHLDITDDYPNSR
NKVVKLSLKPYRFDVYLDNGVYKFVTVKNLDVIKKENYYEVNSKCYEEAKKLKKISNQAEFIASFYKN
DLIKINGELYRVIGVNNDLLNRIEVNMIDITYREYLENMNDKRPPHIIKTIASKTQSIKKYSTDILGNLYE
VKSKKHPQIIKKG
Streptococcus thermophilus CRISPR1 Cas9 (St1Cas9) Nickase (D9A)
(SEQ ID NO: 269)
MSDLVLGLAIGIGSVGVGILNKVTGEIIHKNSRIFPAAQAENNLVRRTNRQGRRLTRRKKHRRVRLNRL
FEESGLITDFTKISINLNPYQLRVKGLTDELSNEELFIALKNMVKHRGISYLDDASDDGNSSIGDYAQIVK
ENSKQLETKTPGQIQLERYQTYGQLRGDFTVEKDGKKHRLINVFPTSAYRSEALRILQTQQEFNPQITDE
FINRYLEILTGKRKYYHGPGNEKSRTDYGRYRTSGETLDNIFGILIGKCTFYPDEFRAAKASYTAQEFNL
LNDLNNLTVPTETKKLSKEQKNQIINYVKNEKAMGPAKLFKYIAKLLSCDVADIKGYRIDKSGKAEIHT
FEAYRKMKTLETLDIEQMDRETLDKLAYVLTLNTEREGIQEALEHEFADGSFSQKQVDELVQFRKANS
SIFGKGWHNFSVKLMMELIPELYETSEEQMTILTRLGKQKTTSSSNKTKYIDEKLLTEEIYNPVVAKSVR
QAIKIVNAAIKEYGDFDNIVIEMARETNEDDEKKAIQKIQKANKDEKDAAMLKAANQYNGKAELPHSV
FHGHKQLATKIRLWHQQGERCLYTGKTISIHDLINNSNQFEVDHILPLSITFDDSLANKVLVYATANQE
KGQRTPYQALDSMDDAWSFRELKAFVRESKTLSNKKKEYLLTEEDISKFDVRKKFIERNLVDTRYASR
VVLNALQEHFRAHKIDTKVSVVRGQFTSQLRRHWGIEKTRDTYHHHAVDALIIAASSQLNLWKKQKN
TLVSYSEDQLLDIETGELISDDEYKESVFKAPYQHFVDTLKSKEFEDSILFSYQVDSKFNRKISDATIYAT
RQAKVGKDKADETYVLGKIKDIYTQDGYDAFMKIYKKDKSKFLMYRHDPQTFEKVIEPILENYPNKQI
NEKGKEVPCNPFLKYKEEHGYIRKYSKKGNGPEIKSLKYYDSKLGNHIDITPKDSNNKVVLQSVSPWR
ADVYFNKTTGKYEILGLKYADLQFEKGTGTYKISQEKYNDIKKKEGVDSDSEFKFTLYKNDLLLVKDT
ETKEQQLFRFLSRTMPKQKHYVELKPYDKQKFEGGEALIKVLGNVANSGQCKKGLGKSNISIYKVRTD
VLGNQHIIKNEGDKPKLDF
Streptococcus thermophilus CRISPR3Cas9 (St3Cas9) Nickase (D10A)
(SEQ ID NO: 2429)
MTKPYSIGLAIGTNSVGWAVITDNYKVPSKKMKVLGNTSKKYIKKNLLGVLLFDSGITAEGRRLKRTA
RRRYTRRRNRILYLQEIFSTEMATLDDAFFQRLDDSFLVPDDKRDSKYPIFGNLVEEKVYHDEFPTIYHL
RKYLADSTKKADLRLVYLALAHMIKYRGHFLIEGEFNSKNNDIQKNFQDFLDTYNAIFESDLSLENSKQ
LEEIVKDKISKLEKKDRILKLFPGEKNSGIFSEFLKLIVGNQADFRKCFNLDEKASLHFSKESYDEDLETL
LGYIGDDYSDVFLKAKKLYDAILLSGFLTVTDNETEAPLSSAMIKRYNEHKEDLALLKEYIRNISLKTYN
EVFKDDTKNGYAGYIDGKTNQEDFYVYLKNLLAEFEGADYFLEKIDREDFLRKQRTFDNGSIPYQIHLQ
EMRAILDKQAKFYPFLAKNKERIEKILTFRIPYYVGPLARGNSDFAWSIRKRNEKITPWNFEDVIDKESS
AEAFINRMTSFDLYLPEEKVLPKHSLLYETFNVYNELTKVRFIAESMRDYQFLDSKQKKDIVRLYFKDK
RKVTDKDIIEYLHAIYGYDGIELKGIEKQFNSSLSTYHDLLNIINDKEFLDDSSNEAIIEEIIHTLTIFEDRE
MIKQRLSKFENIFDKSVLKKLSRRHYTGWGKLSAKLINGIRDEKSGNTILDYLIDDGISNRNFMQLIHDD
ALSFKKKIQKAQIIGDEDKGNIKEVVKSLPGSPAIKKGILQSIKIVDELVKVMGGRKPESIVVEMARENQ
YTNQGKSNSQQRLKRLEKSLKELGSKILKENIPAKLSKIDNNALQNDRLYLYYLQNGKDMYTGDDLDI
DRLSNYDIDHIIPQAFLKDNSIDNKVLVSSASNRGKSDDFPSLEVVKKRKTFWYQLLKSKLISQRKFDNL
TKAERGGLLPEDKAGFIQRQLVETRQITKHVARLLDEKFNNKKDENNRAVRTVKIITLKSTLVSQFRKD
FELYKVREINDFHHAHDAYLNAVIASALLKKYPKLEPEFVYGDYPKYNSFRERKSATEKVYFYSNIMNI
FKKSISLADGRVIERPLIEVNEETGESVWNKESDLATVRRVLSYPQVNVVKKVEEQNHGLDRGKPKGL
FNANLSSKPKPNSNENLVGAKEYLDPKKYGGYAGISNSFAVLVKGTIEKGAKKKITNVLEFQGISILDRI
NYRKDKLNFLLEKGYKDIELIIELPKYSLFELSDGSRRMLASILSTNNKRGEIHKGNQIFLSQKFVKLLYH
AKRISNTINENHRKYVENHKKEFEELFYYILEFNENYVGAKKNGKLLNSAFQSWQNHSIDELCSSFIGPT
GSERKGLFELTSRGSAADFEFLGVKIPRYRDYTPSSLLKDATLIHQSVTGLYETRIDLAKLGEG

In some embodiments, the nucleobase editors useful in the present disclosure comprises: (i) a guide nucleotide sequence-programmable DNA-binding protein domain; and (ii) a deaminase domain. In some embodiments, the deaminase domain of the fusion protein is a cytosine deaminase. In some embodiments, the deaminase is an APOBEC1 deaminase. In some embodiments, the deaminase is a rat APOBEC1. In some embodiments, the deaminase is a human APOBEC1. In some embodiments, the deaminase is an APOBEC2 deaminase. In some embodiments, the deaminase is an APOBEC3A deaminase. In some embodiments, the deaminase is an APOBEC3B deaminase. In some embodiments, the deaminase is an APOBEC3C deaminase. In some embodiments, the deaminase is an APOBEC3D deaminase. In some embodiments, is an APOBEC3F deaminase. In some embodiments, the deaminase is an APOBEC3G deaminase. In some embodiments, the deaminase is an APOBEC3H deaminase. In some embodiments, the deaminase is an APOBEC4 deaminase. In some embodiments, the deaminase is an activation-induced deaminase (AID). In some embodiments, the deaminase is a Lamprey CDA1 (pmCDA1). In some embodiments, the deaminase is a human APOBEC3G or a functional fragment thereof. In some embodiments, the deaminase is an APOBEC3G variant comprising mutations corresponding to the D316R/D317R mutations in the human APOBEC3G. Exemplary, non-limiting cytosine deaminase sequences that may be used in accordance with the methods of the present disclosure are provided in Example 1 below.

In some embodiments, the cytosine deaminase is a wild type deaminase or a deaminase as set forth in SEQ ID NOs: 271-292, 303, and 2483-2494. In some embodiments, the cytosine deaminase domains of the fusion proteins provided herein include fragments of deaminases or proteins homologous to a deaminase. For example, in some embodiments, a deaminase domain comprises a fragment of any of the amino acid sequences set forth in any of SEQ ID NOs: 271-292, 303, and 2483-2494. In some embodiments, a deaminase domain comprises an amino acid sequence homologous to the amino acid sequence set forth in any of SEQ ID NOs: 271-292, 303, and 2483-2494, or an amino acid sequence homologous to a fragment of the amino acid sequence set forth in any of SEQ ID NOs: 271-292, 303, and 2483-2494. In some embodiments, proteins comprising a deaminase, fragments of a deaminase, or homologs of a deaminase are referred to as “deaminase variants.” A deaminase variant shares homology to a deaminase, or a fragment thereof. For example, a deaminase variant is at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% to a wild type deaminase or a deaminase as set forth in any of SEQ ID NOs: 271-292, 303, and 2483-2494. In some embodiments, the deaminase variant comprises a fragment of the deaminase, such that the fragment is at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical to the corresponding fragment of a wild type deaminase or a deaminase as set forth in any of SEQ ID NOs: 271-292, 303, and 2483-2494. In some embodiments, the cytosine deaminase is at least at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical to an APOBEC3G variant as set forth in SEQ ID NO: 291 or SEQ ID NO: 292, and comprises mutations corresponding to the D316E/D317R mutations in SEQ ID NO: 290.

In some embodiments, the cytosine deaminase domain is fused to the N-terminus of the guide nucleotide sequence-programmable DNA-binding protein domain. For example, the fusion protein may have an architecture of NH₂-[cytosine deaminase]-[guide nucleotide sequence-programmable DNA-binding protein domain]-COOH. The “]-[” used in the general architecture above indicates the presence of an optional linker sequence. The term “linker,” as used herein, refers to a chemical group or a molecule linking two molecules or moieties, e.g., two domains of a fusion protein, such as, for example, a dCas9 domain and a cytosine deaminase domain. Typically, the linker is positioned between, or flanked by, two groups, molecules, or other moieties and connected to each one via a covalent bond, thus connecting the two. In some embodiments, the linker is an amino acid or a plurality of amino acids (e.g., a peptide or protein). In some embodiments, the linker is an organic molecule, group, polymer, or chemical moiety. In some embodiments, the linker is 5-100 amino acids in length, for example, 5, 6, 7, 8, 9, 10, 11, 12, 13, 14, 15, 16, 17, 18, 19, 20, 21, 22, 23, 24, 25, 26, 27, 28, 29, 30, 30-35, 35-40, 40-45.45-50, 50-60, 60-70, 70-80, 80-90, 90-100, 100-150, or 150-200 amino acids in length. Longer or shorter linkers are also contemplated.

In some embodiments, the cytosine deaminase domain and the Cas9 domain are fused to each other via a linker. Various linker lengths and flexibilities between the deaminase domain (e.g., APOBEC1) and the Cas9 domain can be employed (e.g., ranging from very flexible linkers of the form (GGGS)ₙ (SEQ ID NO: 2430), (GGGGS)ₙ (SEQ ID NO: 308), (GGS)ₙ (SEQ ID NO: 2467), and (G)ₙ (SEQ ID NO: 2498) to more rigid linkers of the form (EAAAK)ₙ (SEQ ID NO: 309), SGSETPGTSESATPES (SEQ ID NO: 310)) (see, e.g., Guilinger et, al., Nat. Biotechnol. 2014; 32(6): 577-82; the entire contents are incorporated herein by reference), (SGGS)ₙSGSETPGTSESATPES(SGGS)ₙ (SEQ ID NO: 2481), (XP)ₙ, or a combination of any of these, wherein X is any amino acid, and n is independently an integer between 1 and 30, in order to achieve the optimal length for deaminase activity for the specific application. In some embodiments, n is independently 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30, or, if more than one linker or more than one linker motif is present, any combination thereof. In some embodiments, the linker comprises a (GGS)ₙ (SEQ ID NO: 2467) motif, wherein n is 1, 2, 3, 4, 5, 6, 7, 8, 9, 10, 11, 12, 13, 14, or 15. In some embodiments, the linker comprises a (GGS)ₙ (SEQ ID NO: 2467) motif, wherein n is 1, 3, or 7. In some embodiments, the linker comprises the amino acid sequence SGSETPGTSESATPES (SEQ ID NO: 310), also referred to as the XTEN linker. In some embodiments, the linker comprises an amino acid sequence selected from the group including, but not limited to, AGVF (SEQ ID NO: 2499), GFLG (SEQ ID NO: 2500), FK, AL, ALAL (SEQ ID NO: 2501), and ALALA (SEQ ID NO: 2502). In some embodiments, suitable linker motifs and configurations include those described in Chen et al., Fusion protein linkers: property, design and functionality. Adv Drug Deliv Rev. 2013; 65(10):1357-69, which is incorporated herein by reference. In some embodiments, the linker comprises any of the following amino acid sequences: VPFLLEPDNINGKTC (SEQ ID NO: 311), GSAGSAAGSGEF (SEQ ID NO: 312). SIVAQLSRPDPA (SEQ ID NO: 313), MKIIEQLPSA (SEQ ID NO: 314), VRHKLKRVGS (SEQ ID NO: 315), GHGTGSTGSGSS (SEQ ID NO: 316), MSRPDPA (SEQ ID NO: 317), GSAGSAAGSGEF (SEQ ID NO: 312), SGSETPGTSESA (SEQ ID NO: 318), SGSETPGTSESATPEGGSGGS (SEQ ID NO: 319), or GGSM (SEQ ID NO: 320). Any linker provided under the “Linkers” section may be used.

In some embodiments, the nucleobase editor comprises a guide nucleotide sequence-programmable DNA-binding protein domain and an apolipoprotein B mRNA-editing complex 1 (APOBEC1) deaminase domain, where the deaminase domain is fused to the N-terminus of the napDNAbp domain via a linker comprising the amino acid sequence SGSETPGTSESATPES (SEQ ID NO: 310). In some embodiments, the a guide nucleotide sequence-programmable DNA-binding protein domain comprises the amino acid sequence of any of the a guide nucleotide sequence-programmable DNA-binding protein domains provided herein. In some embodiments, the deaminase is rat APOBEC1 (SEQ ID NO: 288). In some embodiments, the deaminase is human APOBEC1 (SEQ ID NO: 286). In some embodiments, the deaminase is pmCDA1 (SEQ ID NO: 289). In some embodiments, the deaminase is human APOBEC3G (SEQ ID NO: 279). In some embodiments, the deaminase is a human APOBEC3G variant of any one of (SEQ ID NOs: 290-292). In some embodiments, the fusion protein comprises a guide nucleotide sequence-programmable DNA-binding protein domain and an apolipoprotein B mRNA-editing complex 1 catalytic polypeptide-like 3G (APOBEC3G) deaminase domain, wherein the deaminase domain is fused to the N-terminus of the a guide nucleotide sequence-programmable DNA-binding protein domain via a linker of any length or composition (e.g., an amino acid sequence, a peptide, a polymer, or a bond). In some embodiments, the linker comprises the amino acid sequence SGSETPGTSESATPES (SEQ ID NO: 310). In some embodiments, the linker comprises the amino acid sequence (SGGS)₂SGSETPGTSESATPES(SGGS)₂ (SEQ ID NO: 2482).

In some embodiments, the fusion protein comprises a guide nucleotide sequence-programmable DNA-binding protein domain and a cytidine deaminase 1 (CDA1) deaminase domain, wherein the deaminase domain is fused to the N-terminus of the guide nucleotide sequence-programmable DNA-binding protein domain via a linker comprising the amino acid sequence SGSETPGTSESATPES (SEQ ID NO: 310). In some embodiments, the linker comprises the amino acid sequence (SGGS)₂SGSETPGTSESATPES(SGGS)₂ (SEQ ID NO: 2482). In some embodiments, the guide nucleotide sequence-programmable DNA-binding protein domain comprises the amino acid sequence of any of the guide nucleotide sequence-programmable DNA-binding protein domains provided herein.

In some embodiments, the fusion protein comprises a guide nucleotide sequence-programmable DNA-binding protein and an activation-induced cytidine deaminase (AID) deaminase domain, where the deaminase domain is fused to the N-terminus of the guide nucleotide sequence-programmable DNA-binding protein domain via a linker comprising the amino acid sequence SGSETPGTSESATPES (SEQ ID NO: 310). In some embodiments, the linker comprises the amino acid sequence (SGGS)₂SGSETPGTSESATPES(SGGS)₂ (SEQ ID NO: 2482). In some embodiments, the guide nucleotide sequence-programmable DNA-binding protein comprises the amino acid sequence of any of the guide nucleotide sequence-programmable DNA-binding protein domains provided herein.

Some aspects of the disclosure are based on the recognition that certain configurations of a guide nucleotide sequence-programmable DNA-binding protein, and a cytidine deaminase domain fused by a linker are useful for efficiently deaminating target cytidine residues. Other aspects of this disclosure relate to the recognition that a nucleobase editing fusion protein with an apolipoprotein B mRNA-editing complex 1 (APOBEC1) deaminase domain fused to the N-terminus of a guide nucleotide sequence-programmable DNA-binding protein via a linker comprising the amino acid sequence SGSETPGTSESATPES (SEQ ID NO: 310) was capable of efficiently deaminating target nucleic acids in a double stranded DNA target molecule. In some embodiments, the fusion protein comprises a guide nucleotide sequence-programmable DNA-binding protein domain and an apolipoprotein B mRNA-editing complex 1 (APOBEC1) deaminase domain, where the deaminase domain is fused to the N-terminus of the napDNAbp via a linker comprising the amino acid sequence SGSETPGTSESATPES (SEQ ID NO: 310). In some embodiments, the fusion protein comprises a guide nucleotide sequence-programmable DNA-binding protein domain and an apolipoprotein B mRNA-editing complex 1 (APOBEC1) deaminase domain, where the deaminase domain is fused to the N-terminus of the napDNAbp via a linker comprising the amino acid sequence (SGGS)₂SGSETPGTSESATPES(SGGS)₂ (SEQ ID NO: 2482).

To successfully edit the desired target C base, the linker between Cas9 and APOBEC may be optimized, as described in Komor et al., Nature, 533, 420-424 (2016), which is incorporated herein by reference. The numbering scheme for base editing is based on the predicted location of the target C within the single-stranded stretch of DNA (R-loop) displaced by a programmable guide RNA sequence occurring when a DNA-binding domain (e.g. Cas9, nCas9, dCas9) binds a genomic site. Conveniently, the sequence immediately surrounding the target C also matches the sequence of the guide RNA. The numbering scheme for base editing is based on a standard 20-mer programmable sequence, and defines position “21” as the first DNA base of the PAM sequence, resulting in position “1” assigned to the first DNA base matching the 5′-end of the 20-mer programmable guide RNA sequence. Therefore, for all Cas9 variants, position “21” is defined as the first base of the PAM sequence (e.g. NGG, NGAN, NGNG, NGAG, NGCG, NNGRRT, NGRRN, NNNRRT, NNNGAT, NNAGAA, NAAAC). When a longer programmable guide RNA sequence is used (e.g. 21-mer) the 5′-end bases are assigned a decreasing negative number starting at “−1”. For other DNA-binding domains that differ in the position of the PAM sequence, or that require no PAM sequence, the programmable guide RNA sequence is used as a reference for numbering. A 3-aa linker results in a 2-5 base editing window (e.g., positions 2, 3, 4, or 5 relative to the PAM sequence at position 21). A 9-aa linker results in a 3-6 base editing window (e.g., positions 3, 4, 5, or 6 relative to the PAM sequence at position 21). A 16-aa linker (e.g., the SGSETPGTSESATPES (SEQ ID NO: 310) linker) results in a 4-7 base editing window (e.g., positions 4, 5, 6, or 7 relative to the PAM sequence at position 21). A 21-aa linker results in a 5-8 base editing window (e.g., positions 5, 6, 7, or 8 relative to the PAM sequence at position 21). Each of these windows can be useful for editing different targeted C bases. For example, the targeted C bases may be at different distances from the adjacent PAM sequence, and by varying the linker length, the precise editing of the desired C base is ensured. One skilled in the art, based on the teachings of CRISPR/Cas9 technology in the art, and in particular the teachings of e.g., in U.S. Pat. No. 9,068,179. US Patent Application Publications US 2015/0166980, published Jul. 18, 2015, US 2015/0166981, published Jul. 18, 2015; US 2015/0166982, published Jul. 18, 2015; US 2015/0166984, published Jul. 18, 2015; and US 2015/0165054, published Jul. 18, 2015; and US Provisional Applications. U.S. Ser. No. 62/245,828, filed Oct. 23, 2015; 62/279,346, filed Jan. 15, 2016; 62/311,763, filed Mar. 22, 2016; 62/322,178, filed Apr. 13, 2016, 62/357,352, filed Jun. 30, 2016, 62,370,700, filed Aug. 3, 2016; 62/398,490, filed Sep. 22, 2016; 62/408,686, filed Oct. 14, 2016; PCT Application PCT/US2016/058344, filed Oct. 22, 2016. U.S. patent application Ser. No. 15/311,852, filed Oct. 22, 2016; and in Komor et al., Nature, Programmable editing of a target base in genomic DNA without double-stranded DNA cleavage, 533, 420-424 (2016), the entire contents of each of which are incorporated herein by reference, will be able to determine the window of editing for his/her purpose, and properly design the linker of the cytosine deaminase-dCas9 protein for the precise targeting of the desired C base.

To successfully edit the desired target C base, appropriate Cas9 domain may be selected to attach to the deaminase domain (e.g., APOBEC1), since different Cas9 domains may lead to different editing windows. For example, APOBEC1-XTEN-SaCas9n-UGI gives a 1-12 base editing window (e.g., positions 1, 2, 3, 4, 5, 6, 7, 8, 9, 10, 11, or 12 relative to the NNNRRT PAM sequence in positions 20-26). One skilled in the art, based on the teachings of CRISPR/Cas9 technology in the art, will be able to determine the editing window and properly determine the required Cas9 homolog and linker attached to the cytosine deaminase for the precise targeting of the desired C base.

In some embodiments, the fusion protein useful in the present disclosure further comprises a uracil glycosylase inhibitor (UGI) domain. A “uracil glycosylase inhibitor” refers to a protein that inhibits the activity of uracil-DNA glycosylase. The C to T base change induced by deamination results in a U:G heteroduplex, which triggers cellular DNA-repair response. Uracil DNA glycosylase (UDG) catalyzes removal of U from DNA in cells and initiates base excision repair, with reversion of the U:G pair to a C:G pair as the most common outcome. Thus, such cellular DNA-repair response may be responsible for the decrease in nucleobase editing efficiency in cells. Uracil DNA Glycosylase Inhibitor (UGI) is known in the art to block UDG activity. As described in Komor et al., Nature (2016), fusing a UGI domain to the cytidine deaminase-dCas9 fusion protein reduced the activity of UDG and significantly enhanced editing efficiency.

Suitable UGI protein and nucleotide sequences are provided herein and additional suitable UGI sequences are known to those in the art, and include, for example, those published in Wang et al., Uracil-DNA glycosylase inhibitor gene of bacteriophage PBS2 encodes a binding protein specific for uracil-DNA glycosylase. J. Biol. Chem. 264:1163-1171(1989); Lundquist et al., Site-directed mutagenesis and characterization of uracil-DNA glycosylase inhibitor protein. Role of specific carboxylic amino acids in complex formation with Escherichia coli uracil-DNA glycosylase. J. Biol. Chem. 272:21408-21419(1997); Ravishankar et al., X-ray analysis of a complex of Escherichia coli uracil DNA glycosylase (EcUDG) with a proteinaceous inhibitor. The structure elucidation of a prokaryotic UDG. Nucleic Acids Res. 26:4880-4887(1998); and Putnam et al., Protein mimicry of DNA from crystal structures of the uracil-DNA glycosylase inhibitor protein and its complex with Escherichia coli uracil-DNA glycosylase. J. Mol. Biol. 287:331-346(1999), each of which is incorporated herein by reference. In some embodiments, the UGI domain comprises the amino acid sequence of SEQ ID NO: 304 without the N-terminal methionine (M). In some embodiments, the UGI protein comprises the following amino acid sequence:

Bacillus phage PBS2 (Bacteriophage PBS2)
Uracil-DNA glycosylase inhibitor
(SEQ ID NO: 304)
MTNLSDIIEKETGKQLVIQESILMLPEEVEEVIGNKPESDILVHTAYDES
TDENVMLLTSDAPEYKPWALVIQDSNGENKIKML

In some embodiments, the UGI protein comprises a wild type UGI or a UGI as set forth in SEQ ID NO: 304. In some embodiments, the UGI proteins useful in the present disclosure include fragments of UGI and proteins homologous to a UGI or a UGI fragment. For example, in some embodiments, a UGI protein comprises a fragment of the amino acid sequence set forth in SEQ ID NO: 304. In some embodiments, a UGI comprises an amino acid sequence homologous to the amino acid sequence set forth in SEQ ID NO: 304 or an amino acid sequence homologous to a fragment of the amino acid sequence set forth in SEQ ID NO: 304. In some embodiments, proteins comprising UGI or fragments of UGI or homologs of UGI or UGI fragments are referred to as “UGI variants.” A UGI variant shares homology to UGI, or a fragment thereof. For example a UGI variant is at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% to a wild type UGI or a UGI as set forth in SEQ ID NO: 304. In some embodiments, the UGI variant comprises a fragment of UGI, such that the fragment is at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical to the corresponding fragment of wild type UGI or the UGI as set forth in SEQ ID NO: 304.

It should be appreciated that additional proteins may be uracil glycosylase inhibitors. For example, other proteins that are capable of inhibiting (e.g., sterically blocking) a uracil-DNA glycosylase base-excision repair enzyme are within the scope of this disclosure. In some embodiments, a uracil glycosylase inhibitor is a protein that binds DNA. In some embodiments, a uracil glycosylase inhibitor is a protein that binds single-stranded DNA. For example, a Erwinia tasmaniensis single-stranded binding protein may also inhibit the activity of uracil glycosylase. In some embodiments, the single-stranded binding protein comprises the amino acid sequence (SEQ ID NO: 305). In some embodiments, a uracil glycosylase inhibitor is a protein that binds uracil. In some embodiments, a uracil glycosylase inhibitor is a protein that binds uracil in DNA. In some embodiments, a uracil glycosylase inhibitor is a catalytically inactive uracil DNA-glycosylase protein. In some embodiments, a uracil glycosylase inhibitor is a catalytically inactive uracil DNA-glycosylase protein that does not excise uracil from the DNA. For example, a uracil glycosylase inhibitor is a UdgX. In some embodiments, the UdgX comprises the amino acid sequence (SEQ ID NO: 306). As another example, a uracil glycosylase inhibitor is a catalytically inactive UDG. In some embodiments, a catalytically inactive UDG comprises the amino acid sequence (SEQ ID NO: 307). It should be appreciated that other uracil glycosylase inhibitors would be apparent to the skilled artisan and are within the scope of this disclosure.

Erwinia tasmaniensis SSB (themostable single-stranded DNA binding
protein)
(SEQ ID NO: 305)
MASRGVNKVILVGNLGQDPEVRYMPNGGAVANITLATSESWRDKQTGETKEKTEWHRVVLFGKLAE
VAGEYLRKGSQVYIEGALQTRKWTDQAGVEKYTTEVVVNVGGTMQMLGGRSQGGGASAGGQNGGS
NNGWGQPQQPQGGNQFSGGAQQQARPQQQPQQNNAPANNEPPIDFDDDIP
UdgX (binds to uracil in DNA but does not excise)
(SEQ ID NO: 306)
MAGAQDFVPHTADLAELAAAAGECRGCGLYRDATQAVFGAGGRSARIMMIGEQPGDKEDLAGLPFV
GPAGRLLDRALEAADIDRDALYVTNAVKHFKFTRAAGGKRRIHKTPSRTEVVACRPWLIAEMTSVEPD
VVVLLGATAAKALLGNDFRVTQHRGEVLHVDDVPGDPALVATVHPSSLLRGPKEERESAFAGLVDDL
RVAADVRP
UDG (catalytically inactive human UDG, binds to uracil in DNA but does
not excise)
(SEQ ID NO: 307)
MIGQKTLYSFFSPSPARKRHAPSPEPAVQGTGVAGVPLESGDAAAIPAKKAPAGQEEPGTPPSSPLSALQ
LDRIQRNKAAALLRLAARNVPVGFGESWKKHLSGEFGKPYFIKLMGFVAEERKHYTVYPPPHQVFTW
TQMCDIKDVKVVILGQEPYHGPNQAHGLCFSVQRPVPPPPSLENIYKELSTDIEDFVHPGHGDLSGWAK
QGVLLLNAVLTVRAHQANSHKERGWEQFTDAVVSWLNQNSNGLVFLLWGSYAQKKGSAIDRKRHH
VLQTAHPSPLSVYRGFFGCRHFSKTNELLQKSGKKPIDWKEL

In some embodiments, the UGI domain is fused to the C-terminus of the dCas9 domain in the fusion protein. Thus, the fusion protein would have an architecture of NH₂-[cytosine deaminase]-[guide nucleotide sequence-programmable DNA-binding protein domain]-[UGI]-COOH. In some embodiments, the UGI domain is fused to the N-terminus of the cytosine deaminase domain. As such, the fusion protein would have an architecture of NH₂-[UGI]-[cytosine deaminase]-[guide nucleotide sequence-programmable DNA-binding protein domain]-COOH. In some embodiments, the UGI domain is fused between the guide nucleotide sequence-programmable DNA-binding protein domain and the cytosine deaminase domain. As such, the fusion protein would have an architecture of NH₂-[cytosine deaminase]-[UGI]-[guide nucleotide sequence-programmable DNA-binding protein domain]-COOH. The linker sequences useful in the present disclosure may also be used for the fusion of the UGI domain to the cytosine deaminase-dCas9 fusion proteins.

In some embodiments, the fusion protein comprises the structure: [cytosine deaminase]-[optional linker sequence]-[guide nucleotide sequence-programmable DNA binding protein]-[optional linker sequence]-[UGI]; [cytosine deaminase]-[optional linker sequence]-[UGI]-[optional linker sequence]-[guide nucleotide sequence-programmable DNA binding protein]; [UGI]-[optional linker sequence]-[cytosine deaminase]-[optional linker sequence]-[guide nucleotide sequence-programmable DNA binding protein]; [UGI]-[optional linker sequence]-[guide nucleotide sequence-programmable DNA binding protein]-[optional linker sequence]-[cytosine deaminase]; [guide nucleotide sequence-programmable DNA binding protein]-[optional linker sequence]-[cytosine deaminase]-[optional linker sequence]-[UGI]; or [guide nucleotide sequence-programmable DNA binding protein]-[optional linker sequence]-[UGI]-[optional linker sequence]-[cytosine deaminase].

In some embodiments, the fusion protein used in the present disclosure comprises the structure:
[cytosine deaminase]-[optional linker sequence]-[Cas9 nickase]-[optional linker sequence]-[UGI];
[cytosine deaminase]-[optional linker sequence]-[UGI]-[optional linker sequence]-[Cas9 nickase];
[UGI]-[optional linker sequence]-[cytosine deaminase]-[optional linker sequence]-[Cas9 nickase];
[UGI]-[optional linker sequence]-[Cas9 nickase]-[optional linker sequence]-[cytosine deaminase];
[Cas9 nickase]-[optional linker sequence]-[cytosine deaminase]-[optional linker sequence]-[UGI]; or
[Cas9 nickase]-[optional linker sequence]-[UGI]-[optional linker sequence]-[cytosine deaminase].

In some embodiments, fusion proteins useful in the present disclosure further comprise a nuclear localization sequence (NLS). In some embodiments, the NLS is fused to the N-terminus of the fusion protein. In some embodiments, the NLS is fused to the C-terminus of the fusion protein. In some embodiments, the NLS is fused to the N-terminus of the UGI protein. In some embodiments, the NLS is fused to the C-terminus of the UGI protein. In some embodiments, the NLS is fused to the N-terminus of the guide nucleotide sequence-programmable DNA-binding protein domain. In some embodiments, the NLS is fused to the C-terminus of the guide nucleotide sequence-programmable DNA-binding protein domain. In some embodiments, the NLS is fused to the N-terminus of the cytosine deaminase. In some embodiments, the NLS is fused to the C-terminus of the deaminase. In some embodiments, the NLS is fused to the fusion protein via one or more linkers. In some embodiments, the NLS is fused to the fusion protein without a linker. Non-limiting, exemplary NLS sequences may be PKKKRKV (SEQ ID NO: 2431) or MDSLLMNRRKFLYQFKNVRWAKGRRETYLC (SEQ ID NO: 2432).

In some embodiments, any of the fusion proteins provided herein comprise a second UGI domain. Fusion proteins comprising two UGI domains are described in U.S. Provisional Application No., U.S. Ser. No. 62/475,830, filed Mar. 23, 2017; 62/490,587; 62/511,934, filed May 26, 2017; 62/551,951, filed Aug. 30, 2017; and Komor et al. (2017) Improved Base Excision Repair Inhibition and Bateriophage Mu Gam Protein Yields C:G-to-T:A base editors with higher efficiency and product purity. Sci Adv, 3: eaao4774; the entire contents of which is incorporated by reference herein. In some embodiments, the second UGI domain comprises a wild-type UGI or a UGI as set forth in SEQ ID NO: 304. In some embodiments, the UGI proteins provided herein include fragments of UGI and proteins homologous to a UGI or a UGI fragment. For example, in some embodiments, the second UGI domain comprises a fragment of the amino acid sequence set forth in SEQ ID NO: 304. In some embodiments, a UGI fragment comprises an amino acid sequence that comprises at least 60%, at least 65%, at least 70%, at least 75%, at least 80%, at least 85%, at least 90%, at least 95%, at least 96%, at least 97%, at least 98%, at least 99%, or at least 99.5% of the amino acid sequence as set forth in SEQ ID NO: 304. In some embodiments, the second UGI domain comprises an amino acid sequence homologous to the amino acid sequence set forth in SEQ ID NO: 304 or an amino acid sequence homologous to a fragment of the amino acid sequence set forth in SEQ ID NO: 304. In some embodiments, proteins comprising UGI or fragments of UGI or homologs of UGI or UGI fragments are referred to as “UGI variants.” A UGI variant shares homology to UGI, or a fragment thereof. For example a UGI variant is at least 70% identical, at least 75% identical, at least 80% identical, at least 85% identical, at least 90% identical, at least 95% identical, at least 96% identical, at least 97% identical, at least 98% identical, at least 99% identical, at least 99.5% identical, or at least 99.9% identical to a wild type UGI or a UGI as set forth in SEQ ID NO: 304. In some embodiments, the UGI variant comprises a fragment of UGI, such that the fragment is at least 70% identical, at least 80% identical, at least 90% identical, at least 95% identical, at least 96% identical, at least 97% identical, at least 98% identical, at least 99% identical, at least 99.5% identical, or at least 99.9% to the corresponding fragment of wild-type UGI or a UGI as set forth in SEQ ID NO: 304.

In some embodiments, the fusion protein comprises the structure:
[deaminase]-[optional linker sequence]-[dCas9]-[optional linker sequence]-[first UGI]-[optional linker sequence]-[second UGI];
[deaminase]-[optional linker sequence]-[Cas9 nickase]-[optional linker sequence]-[first UGI]-[optional linker sequence]-[second UGI]; or
[deaminase]-[optional linker sequence]-[Cas9]-[optional linker sequence]-[first UGI]-[optional linker sequence]-[second UGI].

In some embodiments, the nucleobase editor comprises a guide nucleotide sequence-programmable DNA-binding protein domain and an apolipoprotein B mRNA-editing complex 1 (APOBEC1) deaminase domain, wherein the deaminase domain is fused to the N-terminus of the guide nucleotide sequence-programmable DNA-binding protein domain via a linker comprising the amino acid sequence (SGGS)₂SGSETPGTSESATPES(SGGS)₂ (SEQ ID NO: 2482). In some embodiments, the a guide nucleotide sequence-programmable DNA-binding protein domain comprises the amino acid sequence of any of the a guide nucleotide sequence-programmable DNA-binding protein domains provided herein. In some embodiments, the deaminase is rat APOBEC1 (SEQ ID NO: 288). In some embodiments, the deaminase is human APOBEC1 (SEQ ID NO: 286). In some embodiments, the deaminase is a human APOBEC3G variant of any one of (SEQ ID NOs: 290-292). In some embodiments, the nucleobase editor comprises a first UGI domain fused to the C-terminus of a guide nucleotide sequence-programmable DNA-binding protein domain via a linker comprising the amino acid sequence (GGS)ₙ (SEQ ID NO: 2467), wherein n is 3. In some embodiments, the nucleobase editor comprises a second UGI domain fused to the C-terminus of a first UGI domain via a linker comprising the amino acid sequence (GGS)ₙ (SEQ ID NO: 2467), wherein n is 3.

In some embodiments, the fusion protein comprises the amino acid sequence of SEQ ID NO: 2495. In some embodiments, the fusion protein comprises an amino acid sequence that is at least 70%, at least 75%, at least 80%, at least 85%, at least 90%, at least 95%, at least 96%, at least 97%, at least 98%, at least 99%, or at least 99.5% identical to the amino acid sequence as set forth in SEQ ID NO: 2495.

In some embodiments, any of the fusion proteins provided herein may further comprise a Gam protein. The term “Gam protein,” as used herein, refers generally to proteins capable of binding to one or more ends of a double strand break of a double stranded nucleic acid (e.g., double stranded DNA). In some embodiments, the Gam protein prevents or inhibits degradation of one or more strands of a nucleic acid at the site of the double strand break. In some embodiments, a Gam protein is a naturally-occurring Gam protein from bacteriophage Mu, or a non-naturally occurring variant thereof. Fusion proteins comprising Gam proteins are described in Komor et al. (2017) Improved Base Excision Repair Inhibition and Bateriophage Mu Gam Protein Yields C:G-to-T:A base editors with higher efficiency and product purity. Sci Adv, 3: eaao4774; the entire contents of which is incorporated by reference herein. In some embodiments, the Gam protein comprises an amino acid sequence that is at least 70%, at least 75%, at least 80%, at least 85%, at least 90%, at least 92%, at least 95%, at least 96%, at least 97%, at least 98%, at least 99%, or at least 99.5% identical to the amino acid sequence provided by SEQ ID NO: 2496. In some embodiments, the Gam protein comprises the amino acid sequence of SEQ ID NO: 2496. In some embodiments, the fusion protein (e.g., BE4-Gam of SEQ ID NO: 2497) comprises a Gam protein, wherein the Cas9 domain of BE4 is replaced with any of the Cas9 domains provided herein.

Gam from bacteriophage Mu:
(SEQ ID NO: 2496)
AKPAKRIKSAAAAYVPQNRDAVIIDIKRIGDLQREASRLETEMNDAIAEITEKFAARIAPIKIDIETLSKGVQGWCE
ANRDELINGGKVKTANLVTGDVSWRVRPPSVSIRGMDAVMETLERLGLQRFIRTKQEINKEAILLEPKAVAGVAGIT
VKSGIEDFSIIPFEQEAGI
BE4-Gam:
(SEQ ID NO: 2497)
MAKPAKRIKSAAAAYVPQNRDAVITDIKRIGDLQREASRLETEMNDAIAEITEKFAARIAPIKTDIETLSKGVQGWC
EANRDELINGGKVKTANLVTGDVSWRVRPPSVSIRGMDAVMETLERLGLQRFIRTKQEINKEAILLEPKAVAGVAGI

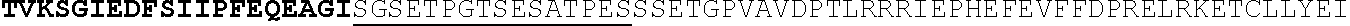

NWGGRHSIWRHISQNINKHVEVNFIEKFTTERYFCPNTRCSITWFLSWSPCGECSRAITEFLSRYPHVTLFIYIARL
YHHADPRNRQGLRDLISSGVTIQIMTEQESGYCWRNFVNYSPSNEAHWPRYPHLWVRLYVLELYCIILGLPPCLNIL

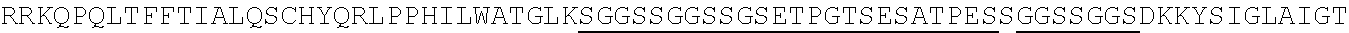

NSVGWAVITDEYKVPSKKFKVLGNTDRHSIKKNLIGALLFDSGETAEATRLKRTARRRYTRRKNRICYLQEIFSNEM
AKVDDSFFERLEESFLVEEDKKHERHPIFGNIVDEVAYHEKYPTIYHLRKKLVDSIDKADLRLIYLALAHMIKFRGH
FLIEGDLNPDNSDVDKLFIQLVQTYNQLFEENPINASGVDAKAILSARLSKSRRLENLIAQLPGEKKNGLFGNLIAL
SLGLTPNEKSNFDLAEDAKLQLSKDTYDDDLDNLLAQIGDQYADLFLAAKNLSDAILLSDILRVNTEITKAPLSASM
IKRYDEHHQDLILLKALVRQQLPEKYKEIFFDQSKNGYAGYIDGGASQEEFYKFIKPILEKMDGTEELLVKLNREDL
LRKQRTEDNGSIPHQIHLGELHAILRRQEDFYPFLKDNREKIEKILTFRIPYYVGPLARGNSRFAWMIRKSEETITP
WNFEEVVDKGASAQSFIERMINEDKNLPNEKVLPKHSLLYEYFTVYNELTKVKYVTEGMRKPAFLSGEQKKAIVDLL
FKTNRKVTVKQLKEDYFKKIECFDSVEISGVEDRENASLGTYHDLLKIIKDKDELDNEENEDILEDIVLTLILFEDR
EMIEERLKTYAHLEDDKVMKQLKRRRYTGWGRLSRKLINGIRDKQSGKTILDELKSDGFANRNFMQLIHDDSLTFKE
DIQKAQVSGQGDSLHEHIANLAGSPAIKKGILQTVKVVDELVKVMGRHKPENIVIEMARENQTTQKGQKNSRERMKR
IEEGIKELGSQILKEHPVENTQLQNEKLYLYYLQNGRDMYVDQELDINRLSDYDVDHIVPQSFLKDDSIDNKVLTRS
DKNRGKSDNVPSEEVVKKMKNYWRQLLNAKLITQRKFDNLTKAERGGLSELDKAGFIKRQLVETRQITKHVAQILDS
RMNTKYDENDKLIREVKVITLKSKLVSDERKDFQFYKVREINNYHHAHDAYLNAVVGTALIKKYPKLESEFVYGDYK
VYDVRKMIAKSEQEIGKATAKYFFYSNIMNFFKTEITLANGEIRKRPLIEINGETGEIVWDKGRDFATVRKVLSMPQ
VNIVKKTEVQTGGFSKESILPKRNSDKLIARKKDWDPKKYGGFDSPTVAYSVLVVAKVEKGKSKKLKSVKELLGITI
MERSSFEKNPIDFLEAKGYKEVKKDLIIKLPKYSLFELENGRKRMLASAGELQKGNELALPSKYVNFLYLASHYEKL
KGSPEDNEQKQLFVEQHKHYLDEIIEQISEFSKRVILADANLDKVLSAYNKHRDKPIREQAENIIHLFTLINLGAPA
AFKYEDITIDRKRYISTKEVLDATLIHQSITGLYETRIDLSQLGGDSGGSGGSGGSINLSDIIEKETGKQLVIQESI
LMLPEEVEEVIGNKPESDILVHIAYDESTDENVMLLTSDAPEYKPWALVIQDSNGENKIKMLSGGSGGSGGSINLSD
IIEKETGKQLVIQESILMLPEEVEEVIGNKPESDILVHIAYDESTDENVMLLTSDAPEYKPWALVIQDSNGENKIKM
LSGGSPKKKRK

### Linkers

In certain embodiments, linkers may be used to link any of the protein or protein domains described herein. The linker may be as simple as a covalent bond, or it may be a polymeric linker many atoms in length. In certain embodiments, the linker is a polypeptide or based on amino acids. In other embodiments, the linker is not peptide-like. In certain embodiments, the linker is a covalent bond (e.g., a carbon-carbon bond, disulfide bond, carbon-heteroatom bond, etc.). In certain embodiments, the linker is a carbon-nitrogen bond of an amide linkage. In certain embodiments, the linker is a cyclic or acyclic, substituted or unsubstituted, branched or unbranched aliphatic or heteroaliphatic linker. In certain embodiments, the linker is polymeric (e.g., polyethylene, polyethylene glycol, polyamide, polyester, etc.). In certain embodiments, the linker comprises a monomer, dimer, or polymer of aminoalkanoic acid. In certain embodiments, the linker comprises an aminoalkanoic acid (e.g., glycine, ethanoic acid, alanine, beta-alanine, 3-aminopropanoic acid, 4-aminobutanoic acid, 5-pentanoic acid, etc.). In certain embodiments, the linker comprises a monomer, dimer, or polymer of aminohexanoic acid (Ahx). In certain embodiments, the linker is based on a carbocyclic moiety (e.g., cyclopentane, cyclohexane). In other embodiments, the linker comprises a polyethylene glycol moiety (PEG). In other embodiments, the linker comprises amino acids. In certain embodiments, the linker comprises a peptide. In certain embodiments, the linker comprises an aryl or heteroaryl moiety. In certain embodiments, the linker is based on a phenyl ring. The linker may include functionalized moieties to facilitate attachment of a nucleophile (e.g., thiol, amino) from the peptide to the linker. Any electrophile may be used as part of the linker. Exemplary electrophiles include, but are not limited to, activated esters, activated amides, Michael acceptors, alkyl halides, aryl halides, acyl halides, and isothiocyanates.

In some embodiments, the linker is an amino acid or a plurality of amino acids (e.g., a peptide or protein). In some embodiments, the linker is a bond (e.g., a covalent bond), an organic molecule, group, polymer, or chemical moiety. In some embodiments, the linker is 5-100 amino acids in length, for example, 5, 6, 7, 8, 9, 10, 11, 12, 13, 14, 15, 16, 17, 18, 19, 20, 21, 22, 23, 24, 25, 26, 27, 28, 29, 30, 30-35, 35-40, 40-45, 45-50, 50-60, 60-70, 70-80, 80-90, 90-100, 100-110, 110-120, 120-130, 130-140, 140-150, or 150-200 amino acids in length. Longer or shorter linkers are also contemplated. In some embodiments, a linker comprises the amino acid sequence SGSETPGTSESATPES (SEQ ID NO: 310), which may also be referred to as the XTEN linker. In some embodiments, a linker comprises the amino acid sequence SGGS (SEQ ID NO: 37). In some embodiments, a linker comprises (SGGS)n (SEQ ID NO: 2468), (GGGS)n (SEQ ID NO: 2430)n (GGGGS)n (SEQ ID NO: 308)n (G)n (SEQ ID NO: 2498)n (EAAAK)n (SEQ ID NO: 40), (GGS)n (SEQ ID NO: 2467), SGSETPGTSESATPES (SEQ ID NO: 310), or (XP)n motif, or a combination of any of these, wherein n is independently an integer between 1 and 30, and wherein X is any amino acid. In some embodiments, n is 1, 2, 3, 4, 5, 6, 7, 8, 9, 10, 11, 12, 13, 14, or 15. In some embodiments, a linker comprises SGSETPGTSESATPES (SEQ ID NO: 10), and SGGS (SEQ ID NO: 37). In some embodiments, a linker comprises SGGSSGSETPGTSESATPESSGGS (SEQ ID NO: 384). In some embodiments, a linker comprises SGGSSGGSSGSETPGTSESATPESSGGSSGGS (SEQ ID NO: 385). In some embodiments, a linker comprises

(SEQ ID NO: 386)
GGSGGSPGSPAGSPTSTEEGTSESATPESGPGTSTEPSEGSAPGSPAGSP
TSTEEGTSTEPSEGSAPGTSTEPSEGSAPGTSESATPESGPGSEPATSGG
SGGS.

### Nucleobase Editor/gRNA Complexes

Some aspects of the present disclosure provide nucleobase editors associated with a guide nucleotide sequence (e.g., a guide RNA or gRNA), gRNAs can exist as a complex of two or more RNAs, or as a single RNA molecule, gRNAs that exist as a single RNA molecule may be referred to as single-guide RNAs (sgRNAs), though “gRNA” is used interchangeably to refer to guide RNAs that exist as either single molecules or as a complex of two or more molecules. Typically, gRNAs that exist as a single RNA species comprise two domains: (1) a domain that shares homology to a target nucleic acid (e.g., and directs binding of the Cas9 complex to the target); and (2) a domain that binds the Cas9 protein. In some embodiments, domain (2) corresponds to a sequence known as a tracrRNA and comprises a stem-loop structure. For example, in some embodiments, domain (2) is identical or homologous to a tracrRNA as provided in Jinek et al., Science 337:816-821(2012), which is incorporated herein by reference. Other examples of gRNAs (e.g., those including domain 2) can be found in U.S. Provisional Patent Application, U.S. Ser. No. 61/874,682, filed Sep. 6, 2013, entitled “Switchable Cas9 Nucleases And Uses Thereof,” and U.S. Provisional Patent Application, U.S. Ser. No. 61/874,746, filed Sep. 6, 2013, entitled “Delivery System For Functional Nucleases,” each of which is incorporated herein by reference. The gRNA comprises a nucleotide sequence that complements a target site, which mediates binding of the nuclease/RNA complex to said target site, providing the sequence specificity of the nuclease:RNA complex. These proteins are able to be targeted, in principle, to any sequence specified by the guide RNA. Methods of using RNA-programmable nucleases, such as Cas9, for site-specific cleavage (e.g., to modify a genome) are known in the art (see e.g., Cong, L. et al. Science 339, 819-823 (2013): Mali. P. et al. Science 339, 823-826 (2013); Hwang, W. Y. et al. Nature Biotechnology 31, 227-229 (2013); Jinek, M. et al. eLife 2, e00471 (2013); Dicarlo, J. E. et al. Nucleic acids research (2013): Jiang, W. et al. Nature biotechnology 31, 233-239 (2013); each of which is incorporated herein by reference). In particular, examples of guide nucleotide sequences (e.g., sgRNAs) that may be used to target the fusion proteins useful in the present disclosure to its target sequence to deaminate the targeted C bases are described in Komor et al., Nature, 533, 420-424 (2016), which is incorporated herein by reference.

The specific structure of the guide nucleotide sequences (e.g., sgRNAs) depends on its target sequence and the relative distance of a PAM sequence downstream of the target sequence. One skilled in the art will understand that no unifying structure of guide nucleotide sequence is given, because the target sequences are different for each and every C targeted to be deaminated.

However, the present disclosure provides guidance on how to design the guide nucleotide sequence, e.g., a sgRNA, so that one skilled in the art may use such teachings to target a sequence of interest. A gRNA typically comprises a tracrRNA framework allowing for Cas9 binding, and a guide sequence, which confers sequence specificity to fusion proteins disclosed herein. In some embodiments, the guide RNA comprises a structure 5′-[guide sequence]-tracrRNA-3′. Non-limiting, exemplary tracrRNA sequences are shown in Table 13.

TABLE 13
tracrRNA othologues and sequences

| | | SEQ |
|---|---|---|
| | | ID |
| Organism | tracrRNA sequence | NO. |
| S. pyogenes | GUUUAAGAGCUAUGCUGGAAAGCCACGGUGAAAAAGUUCA | 322 |
| | ACUAUUGCCUGAUCGGAAUAAAUUUGAACGAUACGACAGU | |
| | CGGUGCUUUUUUU | |
| S. pyogenes | GUUUAAGAGCUAGAAAUAGCAAGUUUAAAUAAGGCUAGUC | 323 |
| | CGUUAUCAACUUGAAAAAGUGGCACCGAGUCGGUGCUUUU | |
| | UU | |
| S. thermophilus | GUUUUUGUACUCUCAAGAUUCAAUAAUCUUGCAGAAGCUA | 324 |
| CRISPR1 | CAAAGAUAAGGCUUCAUGCCGAAAUCAACACCCUGUCAUUU | |
| | UAUGGCAGGGUGUUUU | |
| S. thermophilus | GUUUUAGAGCUGUGUUGUUUGUUAAAACAACACAGCGAGU | 325 |
| CRISPR3 | UAAAAUAAGGCUUAGUCCGUACUCAACUUGAAAAGGUGGC | |
| | ACCGAUUCGGUGUUUUU | |
| C. jejuni | AAGAAAUUUAAAAAGGGACUAAAAUAAAGAGUUUGCGGGA | 326 |
| | CUCUGCGGGGUUACAAUCCCCUAAAACCGCUUUU | |
| F. novicida | AUCUAAAAUUAUAAAUGUACCAAAUAAUUAAUGCUCUGUA | 327 |
| | AUCAUUUAAAAGUAUUUUGAACGGACCUCUGUUUGACACG | |
| | UCUGAAUAACUAAAA | |
| S. thermophilus2 | UGUAAGGGACGCCUUACACAGUUACUUAAAUCUUGCAGAA | 328 |
| | GCUACAAAGAUAAGGCUUCAUGCCGAAAUCAACACCCUGUC | |
| | AUUUUAUGGCAGGGUGUUUUCGUUAUUU | |
| M. mobile | UGUAUUUCGAAAUACAGAUGUACAGUUAAGAAUACAUAAG | 329 |
| | AAUGAUACAUCACUAAAAAAAGGCUUUAUGCCGUAACUAC | |
| | UACUUAUUUUCAAAAUAAGUAGUUUUUUUU | |
| L. innocua | AUUGUUAGUAUUCAAAAUAACAUAGCAAGUUAAAAUAAGG | 330 |
| | CUUUGUCCGUUAUCAACUUUUAAUUAAGUAGCGCUGUUUC | |
| | GGCGCUUUUUUU | |
| S. pyogenes | GUUGGAACCAUUCAAAACAGCAUAGCAAGUUAAAAUAAGG | 331 |
| | CUAGUCCGUUAUCAACUUGAAAAAGUGGCACCGAGUCGGU | |
| | GCUUUUUUU | |
| S. mutans | GUUGGAAUCAUUCGAAACAACACAGCAAGUUAAAAUAAGG | 332 |
| | CAGUGAUUUUUAAUCCAGUCCGUACACAACUUGAAAAAGU | |
| | GCGCACCGAUUCGGUGCUUUUUUAUUU | |
| S. thermophilus | UUGUGGUUUGAAACCAUUCGAAACAACACAGCGAGUUAAA | 333 |
| | AUAAGGCUUAGUCCGUACUCAACUUGAAAAGGUGGCACCG | |
| | AUUCGGUGUUUUUUUU | |
| N. meningitidis | ACAUAUUGUCGCACUGCGAAAUGAGAACCGUUGCUACAAU | 334 |
| | AAGGCCGUCUGAAAAGAUGUGCCGCAACGCUCUGCCCCUUA | |
| | AAGCUUCUGCUUUAAGGGGCA | |
| P. multocida | GCAUAUUGUUGCACUGCGAAAUGAGAGACGUUGCUACAAU | 335 |
| | AAGGCUUCUGAAAAGAAUGACCGUAACGCUCUGCCCCUUGU | |
| | GAUUCUUAAUUGCAAGGGGCAUCGUUUUU | |

The guide sequence of the gRNA comprises a sequence that is complementary to the target sequence. The guide sequence is typically about 20 nucleotides long. For example, the guide sequence may be approximately 15-25 nucleotides long. In some embodiments, the guide sequence is 15, 16, 17, 18, 19, 20, 21, 22, 23, 24, or 25 nucleotides long. In some embodiments, the guide sequence is more than 25 nucleotides long. Such suitable guide RNA sequences typically comprise guide sequences that are complementary to a nucleic sequence within 50 nucleotides upstream or downstream of the target nucleotide to be edited.

In some embodiments, the guide RNA is about 15-100 nucleotides long and comprises a sequence of at least 10 contiguous nucleotides that is complementary to a target sequence. In some embodiments, the guide RNA is 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides long. In some embodiments, the guide RNA comprises a sequence of 15, 16, 17, 18, 19, 20, 21, 22, 23, 24, 25, 26, 27, 28, 29, 30, 31, 32, 33, 34, 35, 36, 37, 38, 39, or 40 contiguous nucleotides that is complementary to a target sequence.

To edit the genes involved in pain propagation (e.g., ion channels in DRG neurons) using the methods described herein, the nucleobase editor and/or the guide nucleotide sequence is introduced into the cell (e.g., a DRG neuron) where the editing is to occur. In some embodiments, nucleic acid molecules (e.g., expression vectors) encoding the nucleobase editors and/or the guide nucleotide sequences are delivered into the cell, resulting in co-expression of the nucleobase editor(s) and/or the guide nucleotide sequence(s) in the cell. The nucleic acid molecules encoding the nucleobase editors and/or the guide nucleotide sequences may be delivered into the cell using any methods known in the art, e.g., transfection (e.g., transfection mediated by cationic liposomes), and transduction (e.g., via viral infection). In some embodiments, a nucleobase editor/gRNA complex is delivered. Methods of delivering a protein to a cell are familiar to those skilled in the art. For example, the nucleobase editor in complex with a gRNA may be associated with a supercharged or cell-penetrating protein or peptide, which facilitates its entry into a cell (e.g., as described in PCT Application Publication WO 2010/129023, published Nov. 11, 2010, and US Patent Application Publication US 2015/0071906, published Mar. 12, 2015, each of which is incorporated herein by reference). In some embodiments, the isolated nucleobase editor in complex with a gRNA is delivered to a cell using a cationic transfection reagent, e.g., the Lipofectamine CRISPRMAX Cas9 Transfection Reagent from Thermofisher Scientific. In some embodiments, the nucleobase editor and the gRNA may be delivered separately. Other suitable delivery methods may also be used. e.g., AAV mediated gene transfer. Strategies for delivery a genome editing agent (e.g., the nucleobase editor) using AAV have been described. e.g., in Zetsche et al., Nature Biotechnology 33, 139-142 (2015), incorporated herein by reference. Delivery of a split Cas9 using AAV has also been described, e.g., in Truong et al., Nucl. Acids Res. 43, 6450 (2016), and U.S. Provisional Application 62/408,575, filed Oct. 14, 2016, each of which is incorporated herein by reference.

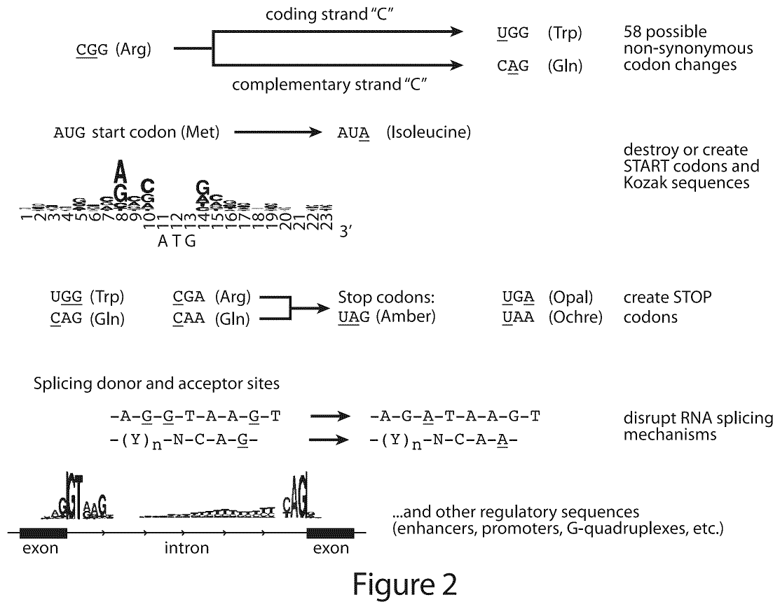

In some embodiments, the genome editing agents (e.g., nucleobase editors) are delivered to neurons (e.g., DRG neurons) using neurotropic viral delivery vectors. Using neurotropic viral delivery vectors to deliver the genome editing agent enables genome editing treatments aimed at the site(s) of pain, despite the fact that the genetic material within the nuclei of DRG neurons is quite distant and indistinguishable from unrelated cells within each ganglion (e.g., as shown in FIG. 2).

In some embodiments, the neurotropic viral delivery vector is derived from a Herpes Simplex Virus 1 (HSV-1), which targets nerve endings in vivo and usurps retrograde axon transport to move the viral DNA up to the cell body of DRG neurons (e.g., as described in Smith et al., Annual Review of Microbiology, 66, 153-176, 2012, which is incorporated herein by reference). In addition, HSV-1 derived vectors allows packaging a large double-stranded DNA genome (>150 kbp), which can easily accommodate an expression construct for any programmable genome-editing enzyme (4-5 kbp), multiple guide-RNAs, and regulatory sequences. In some embodiments, the nucleotide sequences encoding the nucleobase editor and/or the gRNA is inserted into a neurotropic viral delivery vector (e.g., a HSV-1 derived vector) by replacing non-essential genes of the virus (e.g., HSV-1). Non-limiting examples of neurotropic viruses that may be used for the delivery of the genome editing agents described herein include the broader herpesviridae group, varicella-zoster, pseudorabies, cytomegalovirus, Epstein-Barr viruses, encephalitis viruses, polio, coxsackie, echo, mumps, measles, and rabies viruses. Evolved AAV that are neurotropic have also been described (e.g., Nature Biotechnology 34, 204-209 (2016), which is incorporated herein by reference) and may be used in accordance with the present disclosure. Delivery of a split Cas9 using AAV has also been described, e.g., in Truong et al., Nucl. Acids Res. 43, 6450 (2016), and US Provisional Application, U.S. Ser. No. 62/408,575, filed Oct. 14, 2016, each of which is incorporated herein by reference.

In some embodiments, the expression of the genome editing agents (e.g., nucleobase editors and/or gRNAs) is driven by a neuron-specific promoter, such that the genome editing agent is expressed specifically in neurons. Non-limiting examples of neuron-specific promoters that may be used in accordance with the present disclosure include: human synapsin I (SYN) promoter (e.g., as described in Li et al., Proc Natl Acad Sci USA 1993; 90: 1460-1464, incorporated herein by reference), mouse calcium/calmodulin-dependent protein kinase II (CaMKII) promoter (e.g., as described in Mayford et al., Proc Natl Acad Sci USA 1996; 93: 13250-13255, incorporated herein by reference), rat tubulin alpha I (Ta1) promoter (e.g., as described in Gloster et al., J Neurosci 1994; 14: 7319-7330, incorporated herein by reference), rat neuron-specific enolase (NSE) promoter (e.g., as described in Forss-Petter et al, Neuron 1990; 5: 187-197, incorporated herein by reference), and human platelet-derived growth factor-beta chain (PDGF) promoter (e.g., as described in Sasahara et al, Cell 1991; 64: 217-227, incorporated herein by reference). In some embodiments, the gRNA sequence is engineered such that it targets the genome editing agent (e.g., the nucleobase editor) to a target gene encoding an ion channel that is only expressed in neurons, thus minimizing or eliminating the effect on other types of tissues (i.e., enhanced specificity).

### Compositions

Aspects of the present disclosure relate to compositions that may be used for pain suppression. Such compositions comprise any of the genome editing agents (e.g., the nucleobase editor and/or gRNA) or nucleic acids (e.g., DNA, RNA) encoding the genome editing agent (e.g., a neurotropic viral delivery vector) described herein. In some embodiments, the composition is administered to a subject for pain suppression.

In some embodiments, the composition further comprises a pharmaceutically acceptable carrier. As used here, the term “pharmaceutically acceptable carrier” means a pharmaceutically acceptable material, composition or vehicle, such as a liquid or solid filler, diluent, excipient, manufacturing aid (e.g., lubricant, tale magnesium, calcium or zinc stearate, or steric acid), or solvent encapsulating material, involved in carrying or transporting the compound from one site (e.g., the delivery site) of the body, to another site (e.g., organ, tissue or portion of the body). A pharmaceutically acceptable carrier is “acceptable” in the sense of being compatible with the other ingredients of the formulation and not injurious to the tissue of the subject (e.g., physiologically compatible, sterile, physiologic pH, etc.). Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides, carbohydrates, and amino acids; (23) serum component, such as serum albumin, HDL, and LDL; (22) C2-C12 alcohols, such as ethanol; and (23) other non-toxic compatible substances employed in pharmaceutical formulations. Wetting agents, coloring agents, release agents, coating agents, sweetening agents, flavoring agents, perfuming agents, preservative and antioxidants can also be present in the formulation. The terms such as “excipient”, “carrier”, “pharmaceutically acceptable carrier” or the like are used interchangeably herein.

Suitable routes of administrating the composition for pain suppression include, without limitation: topical, subcutaneous, transdermal, intradermal, intralesional, intraarticular, intraperitoneal, intravesical, transmucosal, gingival, intradental, intracochlear, transtympanic, intraorgan, epidural, intrathecal, intramuscular, intravenous, intravascular, intraosseus, periocular, intratumoral, intracerebral, and intracerebroventricular administration.

In some embodiments, the composition for pain suppression is administered locally to the site of pain (e.g., via tropical administration or injection). In some embodiments, the localized volume of treatment is 1 μm³ to 1 dm³ (e.g., 1, 10 μm³, 100 μm³, 1000 μm³, 10000 μm³, or 1 dm³).

In some embodiments, the composition for pain suppression is administered to a subject by injection, by means of a catheter, by means of a suppository, or by means of an implant, the implant being of a porous, non-porous, or gelatinous material, including a membrane, such as a sialastic membrane, or a fiber.

In other embodiments, the compositions for pain suppression are delivered in a controlled release system. In one embodiment, a pump may be used (see. e.g., Langer, 1990, Science 249:1527-1533; Sefton, 1989, CRC Crit. Ref. Biomed. Eng. 14:201; Buchwald et al., 1980, Surgery 88:507; Saudek et al., 1989, N. Engl. J. Med. 321:574). In another embodiment, polymeric materials can be used. (See, e.g., Medical Applications of Controlled Release (Langer and Wise eds., CRC Press, Boca Raton, Fla., 1974); Controlled Drug Bioavailability, Drug Product Design and Performance (Smolen and Ball eds., Wiley. New York, 1984); Ranger and Peppas, 1983, Macromol. Sci. Rev. Macromol. Chem. 23:61. See also Levy et al., 1985, Science 228:190; During et al., 1989, Ann. Neurol. 25:351; Howard et al., 1989. J. Neurosurg. 71:105.) Other controlled release systems are discussed, for example, in Langer, supra.

In some embodiments, the composition is formulated in accordance with routine procedures as a composition adapted for intravenous or subcutaneous administration to a subject, e.g., a human. In some embodiments, compositions for administration by injection are solutions in sterile isotonic aqueous buffer. Where necessary, the pharmaceutical can also include a solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the pharmaceutical is to be administered by infusion, it can be dispensed with an infusion bottle containing sterile pharmaceutical grade water or saline. Where the pharmaceutical is administered by injection, an ampoule of sterile water for injection or saline can be provided so that the ingredients can be mixed prior to administration.

A composition for systemic administration may be a liquid. e.g., sterile saline, lactated Ringer's or Hank's solution. In addition, the pharmaceutical composition can be in solid forms and re-dissolved or suspended immediately prior to use. Lyophilized forms are also contemplated.

The pharmaceutical composition can be contained within a lipid particle or vesicle, such as a liposome or microcrystal, which is also suitable for parenteral administration. The particles can be of any suitable structure, such as unilamellar or plurilamellar, so long as compositions are contained therein. Compounds can be entrapped in “stabilized plasmid-lipid particles” (SPLP) containing the fusogenic lipid dioleoylphosphatidylethanolamine (DOPE), low levels (5-10 mol %) of cationic lipid, and stabilized by a polyethyleneglycol (PEG) coating (Zhang Y. P. et al., Gene Ther. 1999, 6:1438-47). Positively charged lipids such as N-[1-(2,3-dioleoyloxi)propyl]-N,N,N-trimethyl-amoniummethylsulfate, or “DOTAP,” are particularly preferred for such particles and vesicles. The preparation of such lipid particles is well known. See, e.g., U.S. Pat. Nos. 4,880,635; 4,906,477; 4,911,928; 4,917,951; 4,920,016; and 4,921.757; each of which is incorporated herein by reference.

The compositions of this disclosure may be administered or packaged as a unit dose, for example. The term “unit dose” when used in reference to a pharmaceutical composition of the present disclosure refers to physically discrete units suitable as unitary dosage for the subject, each unit containing a predetermined quantity of active material calculated to produce the desired therapeutic effect in association with the required diluent; i.e., carrier, or vehicle.

Further, the compositions of the present disclosure may be assembled into kits. In some embodiments, the kit comprises nucleic acid vectors for the expression of the genome-editing agents useful in the present disclosure. In some embodiments, the kit further comprises appropriate guide nucleotide sequences (e.g., gRNAs), or nucleic acid vectors for the expression of such guide nucleotide sequences, for targeting the nucleobase editor to the desired target sequence.

The kit described herein may include one or more containers housing components for performing the methods described herein and optionally instructions of uses. Any of the kit described herein may further comprise components needed for performing the assay methods. Each component of the kits, where applicable, may be provided in liquid form (e.g., in solution), or in solid form. (e.g., a dry powder). In certain cases, some of the components may be reconstitutable or otherwise processible (e.g., to an active form), for example, by the addition of a suitable solvent or other species (for example, water or certain organic solvents), which may or may not be provided with the kit.

In some embodiments, the kits may optionally include instructions and/or promotion for use of the components provided. As used herein, “instructions” can define a component of instruction and/or promotion, and typically involve written instructions on or associated with packaging of the disclosure. Instructions also can include any oral or electronic instructions provided in any manner such that a user will clearly recognize that the instructions are to be associated with the kit, for example, audiovisual (e.g., videotape, DVD, etc.), Internet. and/or web-based communications, etc. The written instructions may be in a form prescribed by a governmental agency regulating the manufacture, use, or sale of pharmaceuticals or biological products, which can also reflect approval by the agency of manufacture, use or sale for animal administration. As used herein, “promoted” includes all methods of doing business including methods of education, hospital and other clinical instruction, scientific inquiry, drug discovery or development, academic research, pharmaceutical industry activity including pharmaceutical sales, and any advertising or other promotional activity including written, oral and electronic communication of any form, associated with the disclosure. Additionally, the kits may include other components depending on the specific application, as described herein.

The kits may contain any one or more of the components described herein in one or more containers. The components may be prepared sterilely, packaged in a syringe and shipped refrigerated. Alternatively it may be housed in a vial or other container for storage. A second container may have other components prepared sterilely. Alternatively the kits may include the active agents premixed and shipped in a vial, tube, or other container.

The kits may have a variety of forms, such as a blister pouch, a shrink wrapped pouch, a vacuum sealable pouch, a sealable thermoformed tray, or a similar pouch or tray form, with the accessories loosely packed within the pouch, one or more tubes, containers, a box or a bag. The kits may be sterilized after the accessories are added, thereby allowing the individual accessories in the container to be otherwise unwrapped. The kits can be sterilized using any appropriate sterilization techniques, such as radiation sterilization, heat sterilization, or other sterilization methods known in the art. The kits may also include other components, depending on the specific application, for example, containers, cell media, salts, buffers, reagents, syringes, needles, a fabric, such as gauze, for applying or removing a disinfecting agent, disposable gloves, a support for the agents prior to administration, etc.

### Therapeutics

The compositions described herein, may be administered to a subject in need thereof, in a therapeutically effective amount, for the suppression of pain. In some embodiments, the pain is chronic pain. “Chronic pain” is pain that lasts a long time. Types of pain that may be treated using the pain suppression strategies described herein include, without limitation: pain associated with a condition such as cancer pain, tumor pressure, bone metastasis, chemotherapy peripheral neuropathy, radiculopathy (sciatica, lumbar, cervical, failed back surgery syndrome), piriformis syndrome, phantom pain, arachnoiditis, fibromyalgia, facet joint mediated pain, sympathetically-mediated pain syndrome such as complex regional pain syndromes (crps), sacroiliac (si) joint mediated pain, meralgia paresthetica, localized myofacial pain syndromes-myofacial trigger points, diffuse myofacial pain syndrome, post-herpetic neuralgia, trigeminal neuralgia, glossopharyngeal neuralgia, scar pain (post-epesiotomy, post-hernia repair, post-surgery, post-radiotherapy), vulvodynia, vaginismus, levator ani syndrome, chronic prostatitis, interstitial cystitis, first bite syndrome, rheumatoid arthritis pain, osteoarthritis pain, atypical odontalgia, phantom tooth pain, neuropathic orofacial pain, and atypical facial pain, nerve block procedures (alternative to neurolytic, neurectomy, radiation, radiofrequency ablation). In some embodiments, the pain is neuropathic pain, allodynia, hyperalgesia, dysesthesia, causalgia, neuralgia, primary erythermalgia, or arthralgia.

A “therapeutically effective amount” as used herein refers to the amount of each therapeutic agent of the present disclosure required to confer therapeutic effect on the subject, either alone or in combination with one or more other therapeutic agents. Effective amounts vary, as recognized by those skilled in the art, depending on the particular condition being treated, the severity of the condition, the individual subject parameters including age, physical condition, size, gender and weight, the duration of the treatment, the nature of concurrent therapy (if any), the specific route of administration and like factors within the knowledge and expertise of the health practitioner. These factors are well known to those of ordinary skill in the art and can be addressed with no more than routine experimentation. It is generally preferred that a maximum dose of the individual components or combinations thereof be used, that is, the highest safe dose according to sound medical judgment. It will be understood by those of ordinary skill in the art, however, that a subject may insist upon a lower dose or tolerable dose for medical reasons, psychological reasons or for virtually any other reasons. Empirical considerations, such as the half-life, generally will contribute to the determination of the dosage.

Frequency of administration may be determined and adjusted over the course of therapy, and is generally, but not necessarily, based on treatment and/or suppression and/or amelioration and/or delay of a disease. Alternatively, sustained continuous release formulations of a genome-editing may be appropriate. Various formulations and devices for achieving sustained release are known in the art. In some embodiments, dosage is daily, every other day, every three days, every four days, every five days, or every six days. In some embodiments, dosing frequency is once every week, every 2 weeks, every 4 weeks, every 5 weeks, every 6 weeks, every 7 weeks, every 8 weeks, every 9 weeks, or every 10 weeks; or once every month, every 2 months, or every 3 months, or longer. The progress of this therapy is easily monitored by conventional techniques and assays.

The dosing regimen can vary over time. In some embodiments, for an adult subject of normal weight, doses ranging from about 0.01 to 1000 mg/kg may be administered. In some embodiments, the dose is between 1 to 200 mg. The particular dosage regimen. i.e., dose, timing and repetition, will depend on the particular subject and that subject's medical history, as well as the properties of the agents.

For the purpose of the present disclosure, the appropriate dosage of a genome-editing agent as described herein will depend on the specific agent (or compositions thereof) employed, the formulation and route of administration, the type and severity of the disease, whether the genome-editing agent is administered for preventive or therapeutic purposes, previous therapy, the subject's clinical history and response to the antagonist, and the discretion of the attending physician. Typically the clinician will administer a genome-editing agent until a dosage is reached that achieves the desired result.

As used herein, the term “treating” refers to the application or administration of a genome-editing agent described herein or a composition comprising such genome-editing agent to a subject in need thereof. Alleviating a disease includes delaying the development or progression of the disease, or reducing disease severity. Alleviating the disease does not necessarily require curative results.

“Development” or “progression” of a disease means initial manifestations and/or ensuing progression of the disease. Development of the disease can be detectable and assessed using standard clinical techniques as well known in the art. However, development also refers to progression that may be undetectable. For purpose of this disclosure, development or progression refers to the biological course of the symptoms. “Development” includes occurrence, recurrence, and onset. As used herein “onset” or “occurrence” of a disease includes initial onset and/or recurrence.

The term “parenteral” as used herein includes subcutaneous, intracutaneous, intravenous, intramuscular, intraarticular, intraarterial, intrasynovial, intrasternal, intrathecal, intralesional, and intracranial injection or infusion techniques. In addition, it can be administered to the subject via injectable depot routes of administration such as using 1-, 3-, or 6-month depot injectable or biodegradable materials and methods.

Without further elaboration, it is believed that one skilled in the art can, based on the above description, utilize the present disclosure to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### EXAMPLES

In order that the invention described herein may be more fully understood, the following examples are set forth. The synthetic examples described in this application are offered to illustrate the compounds and methods provided herein and are not to be construed in any way as limiting their scope.

### Example 1: Guide Nucleotide Sequence-Programmable DNA-Binding Protein Domains, Deaminases, and Base Editors

Non-limiting examples of suitable guide nucleotide sequence-programmable DNA-binding protein domain s are provided. The disclosure provides Cas9 variants, for example, Cas9 proteins from one or more organisms, which may comprise one or more mutations (e.g., to generate dCas9 or Cas9 nickase). In some embodiments, one or more of the amino acid residues, identified below by an asterisk, of a Cas9 protein may be mutated. In some embodiments, the D10 and/or H840 residues of the amino acid sequence provided in SEQ ID NO: 1, or a corresponding mutation in any of the amino acid sequences provided in SEQ ID NOs: 11-260, are mutated. In some embodiments, the D10 residue of the amino acid sequence provided in SEQ ID NO: 1, or a corresponding mutation in any of the amino acid sequences provided in SEQ ID NOs: 11-260, is mutated to any amino acid residue, except for D. In some embodiments, the D10 residue of the amino acid sequence provided in SEQ ID NO: 1, or a corresponding mutation in any of the amino acid sequences provided in SEQ ID NOs: 11-260, is mutated to an A. In some embodiments, the H840 residue of the amino acid sequence provided in SEQ ID NO: 1, or a corresponding residue in any of the amino acid sequences provided in SEQ ID NOs: 11-260, is an H. In some embodiments, the H840 residue of the amino acid sequence provided in SEQ ID NO: 1, or a corresponding mutation in any of the amino acid sequences provided in SEQ ID NOs: 11-260, is mutated to any amino acid residue, except for H. In some embodiments, the H840 residue of the amino acid sequence provided in SEQ ID NO: 1, or a corresponding mutation in any of the amino acid sequences provided in SEQ ID NOs: 11-260, is mutated to an A. In some embodiments, the D10 residue of the amino acid sequence provided in SEQ ID NO: 1, or a corresponding residue in any of the amino acid sequences provided in SEQ ID NOs: 11-260, is a D.

A number of Cas9 sequences from various species were aligned to determine whether corresponding homologous amino acid residues of D10 and H840 of SEQ ID NO: 1 or SEQ ID NO: 11 can be identified in other Cas9 proteins, allowing the generation of Cas9 variants with corresponding mutations of the homologous amino acid residues. The alignment was carried out using the NCBI Constraint-based Multiple Alignment Tool (COBALT (accessible at st-va.ncbi.nlm.nih.gov/tools/cobalt), with the following parameters. Alignment parameters: Gap penalties −11, −1; End-Gap penalties −5, −1. CDD Parameters: Use RPS BLAST on; Blast E-value 0.003; Find Conserved columns and Recompute on. Query Clustering Parameters: Use query clusters on; Word Size 4; Max cluster distance 0.8; Alphabet Regular.

An exemplary alignment of four Cas9 sequences is provided below. The Cas9 sequences in the alignment are: Sequence 1 (S1): SEQ ID NO: 11|WP_010922251|gi 4992247111 type II CRISPR RNA-guided endonuclease Cas9 [Streptococcus pyogenes]; Sequence 2 (S2): SEQ ID NO: 12|WP_039695303|gi 746743737|type II CRISPR RNA-guided endonuclease Cas9 [Streptococcus gallolyticus]; Sequence 3 (S3): SEQ ID NO: 13|WP_045635197|gi 782887988|type II CRISPR RNA-guided endonuclease Cas9 [Streptococcus mitis]; Sequence 4 (S4): SEQ ID NO: 14|5AXW_A|gi 924443546|Staphylococcus Aureus Cas9. The HNH domain (bold and underlined) and the RuvC domain (boxed) are identified for each of the four sequences. Amino acid residues 10 and 840 in S1 and the homologous amino acids in the aligned sequences are identified with an asterisk following the respective amino acid residue.

| | | | |
|---|---|---|---|
| S1 | 1 | --MDKK-YSIGLD*IGTNSVGWAVITDEYKVESKKFKVLGNTDRESIKENLI--GALLEDSG--ETAEATRLKRTARRRYT | 73 |
| S2 | 1 | --MTKKNYSIGLD*IGTNSVGWAVITDDYKVPAKKMKVIGNTDKKYIKENLL--GALLFDSG--ETAEATRLKRTARRRYT | 74 |
| S3 | 1 | --M-KKGYSIGLD*IGTNSVGFAVITDDYKVESKEMEVLGNTDKRFIKKNLI--GALLFDEG--TTAEARRLKRTARRRYT | 73 |
| S4 | 1 | GSHMKRNYILGLD*IGITSVGYGII--DYET-----------------RDVIDAGVRIFKEANVENNEGRRSKRGARRLKR | 61 |
| S1 | 74 | RRKNRICYLQEIFSNEMAKVDDSFFHRLEESFLVEEDKKHERHPIFGNIVDEVAYHEKYPTIYHLRKKLVDSTDKADLRL | 153 |
| S2 | 75 | RRKNRLRYLQEIFANEIAKVDESFFQRLDESFLTDDDKTEDSHPIFGNKAEEDAYHQKFPTIYHLRKHLADSSEKADLRL | 154 |
| S3 | 74 | RRKNRLRYLQEIFSEEMSKVDSSFFHRLDDSFLIPEDKRESKYPIFATLTEEKEYHKQFPTIYHLRKQLADSKEKTDLRL | 153 |
| S4 | 62 | RRRHRIQRVKKLL--------------FDYNLLTD--------------------HSELSGINPYEARVKGLSQKLSEEE | 107 |
| S1 | 154 | TYLALAHMIKFRGHFLIEGDLNPDNSDVDKLFIQLVQTYNQLFEENPINASGVDAKAILSARLSKSRRLENLIAQLPGEK | 233 |
| S2 | 155 | VYLALAHMIKFRGHFLIEGELNAENTDVQKIFADFVGVYNRTFDDSHLSEITVDVASILTEKISKSRRLENLIKYYPTEK | 234 |
| S3 | 154 | TYLALAHMIKYRGHFLYEEAFDIKNNDIQKIFNEFISIYDNTFEGSSLSGQNAQVEAIFTDKISKSAKRERVLKLFPDEK | 233 |
| S4 | 108 | FSAALLHLAKRRG----------------------VHNVNEVEEDT---------------------------------- | 131 |
| S1 | 234 | KNGLFGNLIALSLGLTPNEKSNFDLAEDAKLQLSKDTYDDDLDNLLAQIGDQYADLFLAAKNLSDAILLSDILRVNTEIT | 313 |
| S2 | 235 | KNTLFGNLIALALGLQPNEKTNFKLSEDAKLQFSKDTYEEDLEELLGKIGDDYADLFTSAKNLYDAILLSGILTVDDNST | 314 |
| S3 | 234 | STGLFSEFLKLIVGNQADFKKHFDLEDKAPLQFSKDTYDEDLENLLGQIGDDFTDLFVSAKKLYDAILLSGILTVTDPST | 313 |
| S4 | 132 | -----GNELS-------------------TKEQISRN------------------------------------------- | 144 |
| S1 | 314 | KAPLSASMIKRYDEHHQDLTLLKALVRQQLPEKYKEIFFDQSKNGYAGYIDGGASQEEFYKFIKPILEKM--DGTEELLV | 391 |
| S2 | 315 | KAPLSASMIKRYVEHHEDLEKLKEFIKANKSELYHDIFKDKNKNGYAGYIENGVKQDEFYKYLKNILSKIKIDGSDYFLD | 394 |
| S3 | 314 | KAPLSASMIERYENHQNDLAALKQFIKNNLPEKYDEVFSDQSKDGYAGYIDGKTTQETFYKYIKNLLSKF--EGTDYFLD | 391 |
| S4 | 145 | ----SKALEEKYVAELQ--------------------------------------------------LERLKKDG----- | 165 |
| S1 | 392 | KLNREDLLRKQRTFDNGSIPHQIHLGELHAILRRQEDFYPFLKDNREKIEKILTFRIPYYVGPLARGNSRFAWMTRKSEE | 471 |
| S2 | 395 | KIEREDFLRKQRTFDNGSIPHQIHLQEMHAILRRQGDYYPFLKEKQDRIEKILTFRIPYYVGPLVRKDSRFAWAEYRSDE | 474 |
| S3 | 392 | KIEREDFLRKQRTFDNGSIPHQIHLQEMNAILRRQGEYYPFLKDNKEKIEKILTFRIPYYVGPLARGNRDFAWLTRNSDE | 471 |
| S4 | 166 | --EVRGSINRFKTSD--------YVKEAKQLLKVQKAYHQLDQSFIDTYIDLLETRRTYYEGP--GEGSPFGW------K | 227 |
| S1 | 472 | TITPWNFEEVVDKGASAQSFIERMTNFDKNLPNEKVLPKHSLLYEYFTVYNELTKVKYVTEGMRKPAFLSGEQKKAIVDL | 551 |
| S2 | 475 | KITPWNFDKVIDKEKSAEKFITRMTLNDLYLPEEKVLPKHSHVYETYAVYNELTKIKYVNEQGKE-SFFDSNMKQEIFDH | 553 |
| S3 | 472 | AIRPWNFEEIVDKASSAEDFINKMTNYDLYLPEEKVLPKHSLLYETFAVYNELTKVKFIAEGLRDYQFLDSGQKKQIVNQ | 551 |
| S4 | 228 | DIKEW---------------YEMLMGHCTYFPEELRSVKYAYNADLYNALNDLNNLVITRDENEK---LEYYEKFQIIEN | 289 |
| S1 | 552 | LFKTNRKVTVKQLKEDYFKKIECFDSVEISGVEDR---FNASLGTYHDLLKIIKDKDFLDNEENEDILEDIVLTLTLFED | 628 |
| S2 | 554 | VFKENRKVTKEKLLNYLNKEFPEYRIKDLIGLDKENKSFNASLGTYHDLKKIL-DKAFLDDKVNEEVIEDIIKTLTLFED | 632 |
| S3 | 552 | LFKENRKVTEKDIIHYLHN-VDGYDGIELKGIEKQ---FNASLSTYHDLLKIIKDKEEMDDAKNEAILENIVHTLTIFED | 627 |
| S4 | 290 | VFKQKKKPTLKQIAKEILVNEEDIKGYRVTSTGKPEF---TNLKVYHDIKDITARKEII---ENAELLDQIAKILTIYQS | 363 |
| S1 | 629 | REMIEERLKTYAHLFDDKVMKQLKR-RRYTGWGRLSRKLINGIRDKQSGKTILDFLKSDGFANRNFMQLIHDDSLTFKED | 707 |
| S2 | 633 | KDMIHERLQKYSDIFTANQLKKLER-RHYTGWGRLSYKLINGIRNKENNKTILDYLIDDGSANRNFMQLINDDTLPFKQI | 711 |
| S3 | 628 | REMIKQRLAQYDSLFDEKVIKALTR-RHYTGWGKLSAKLINGICDKQTGNTILDYLIDDGKINRNFMQLINDDGLSFKEI | 706 |
| S4 | 364 | SEDIQEELTNLNSELTQEEIEQISNLKGYTGTHNLSLKAINLILDE------LWHTNDNQIAIFNRLKLVP--------- | 428 |
| S1 | 708 | 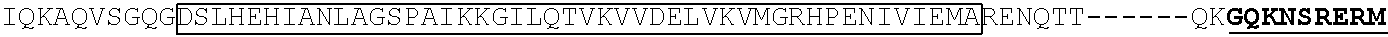 | 781 |
| S2 | 712 | 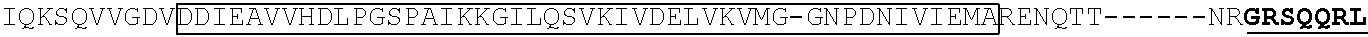 | 784 |
| S3 | 707 | 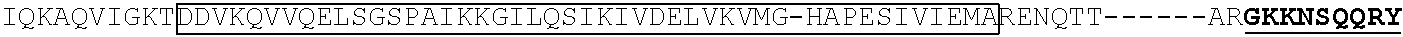 | 779 |
| S4 | 429 | 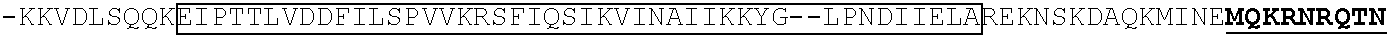 | 505 |
| S1 | 782 | KRIEEGIKELGSQIL-------KEHPVENTQLQNEKLYLYYLQNGRDMYVDQELDINRLSD----YDVDH*IVPQSFLKDD | 850 |
| S2 | 785 | KKLQNSLKELGSNILNEEKPSYIEDKVENSHLQNDQLFLYYIQNGKDMYTGDELDIDHLSD----YDIDH*IIPQAFIKDD | 860 |
| S3 | 780 | KRIEDSLKILASGL---DSNILKENPTDNNQLQNDRLFLYYLQNGKDMYTGEALDINQLSS----YDIDH*IIPQAFIKDD | 852 |
| S4 | 506 | ERIEEIIRTTGK---------------ENAKYLIEKIKLHDMQEGKCLYSLEAIPLEDLLNNPFNYEVDH*IIPRSVSFDN | 570 |
| S1 | 851 | 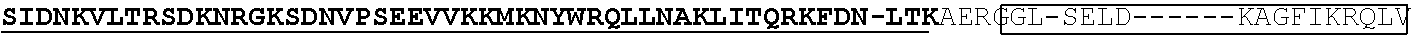 | 922 |
| S2 | 861 | 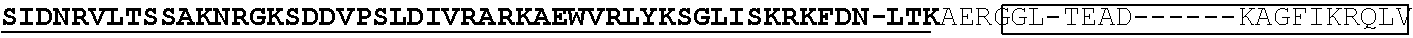 | 932 |
| S3 | 853 | 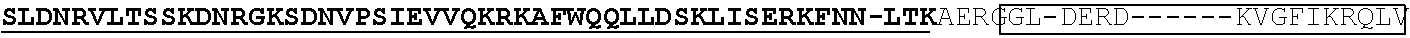 | 924 |
| S4 | 571 | 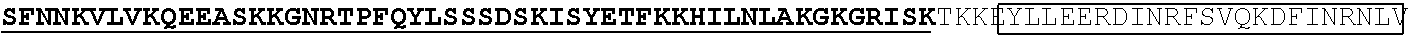 | 650 |
| S1 | 923 | 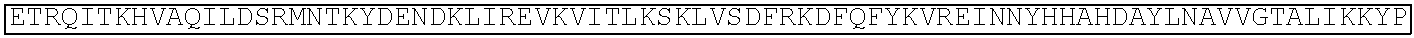 | 1002 |
| S2 | 933 | 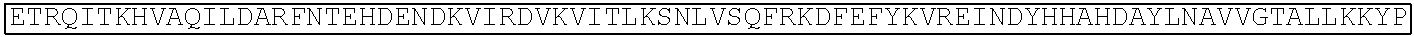 | 1012 |
| S3 | 925 | 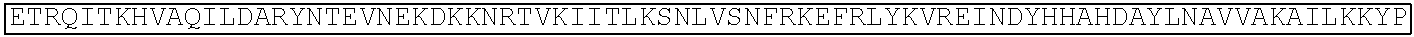 | 1004 |
| S4 | 651 | 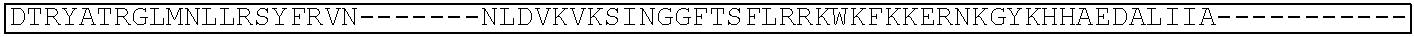 | 712 |
| S1 | 1003 | 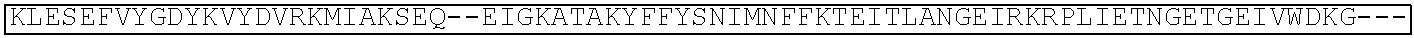 | 1077 |
| S2 | 1013 | 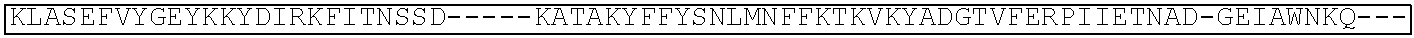 | 1083 |
| S3 | 1005 | 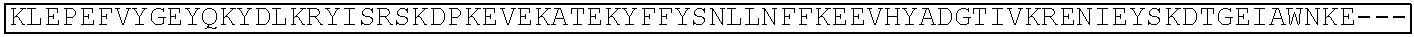 | 1081 |
| S4 | 713 | 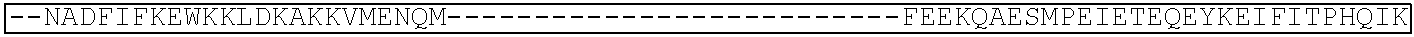 | 764 |
| S1 | 1078 | 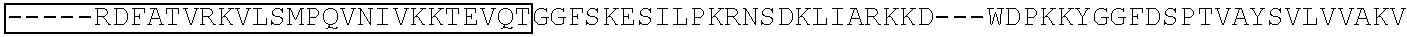 | 1149 |
| S2 | 1084 | 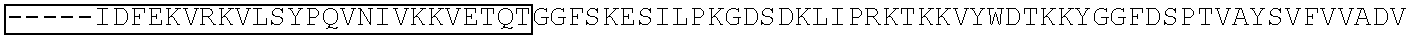 | 1158 |
| S3 | 1082 | 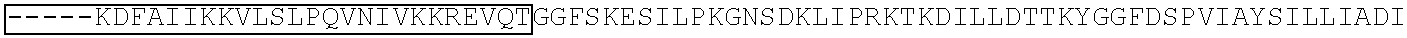 | 1156 |
| S4 | 765 | 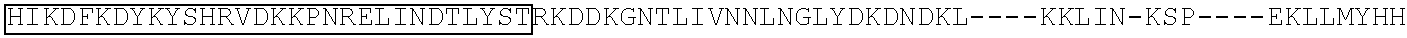 | 835 |
| S1 | 1150 | EKGKSKKLKSVKELLGITIMERSSFEKNPI-DFLEAKG-----YKEVKKDLIIKLPKYSLFELENGRKRMLASAGELQKG | 1223 |
| S2 | 1159 | EKGKAKKLKTVKELVGISIMERSFFEENPV-EFLENKG-----YHNIREDKLIKLPKYSLFEFEGGRRRLLASASELQKG | 1232 |
| S3 | 1157 | EKGKAKKLKTVKTLVGITIMEKAAFEENPI-TFLENKG-----YHNVRKENILCLPKYSLFELENGRRRLLASAKELQKG | 1230 |
| S4 | 836 | DPQTYQKLK--------LIMEQYGDEKNPLYKYYEETGNYLTKYSKKDNGPVIKKIKYYGNKLNAHLDITDDYPNSRNKV | 907 |
| S1 | 1224 | NELALPSKYVNFLYLASHYEKLKGSPEDNEQKQLFVEQHKHYLDEITEQISEFSKRVILADANLDKVLSAYNKH------ | 1297 |
| S2 | 1233 | NEMVLPGYLVELLYHAHRADNF-----NSTEYLNYVSEHKKEFEKVLSCVEDFANLYVDVEKNLSKIRAVADSM------ | 1301 |
| S3 | 1231 | NEIVLPVYLTTLLYHSKNVHKL-----DEPGHLEYIQKHRNEFKDLLNLVSEFSQKYVLADANLEKIKSLYADN------ | 1299 |
| S4 | 908 | VKLSLKPYRFD-VYLDNGVYKFV-----TVKNLDVIK--KENYYEVNSKAYEEAKKLKKISNQAEFIASFYNNDLIKING | 979 |
| S1 | 1298 | RDKPIREQAENITHLFTLTNLGAPAAFKYFDTTIDRKRYTSTKEVLDATLIHQSIT--------GLYETRI----DLSQL | 1365 |
| S2 | 1302 | DNFSIEEISNSFINLLTLTALGAPADFNFLGEKIPRKRYTSTKECLNATLIHQSIT--------GLYETRI----DLSKL | 1369 |
| S3 | 1300 | EQADIEILANSFINLLTFTALGAPAAFKFFGKDIDRKRYTTVSEILNATLIHQSIT--------GLYETWI----DLSKL | 1367 |
| S4 | 980 | ELYRVIGVNNDLLNRIEVNMIDITYR-EYLENMNDKRPPRIIKTIASKT---QSIKKYSTDILGNLYEVKSKKHPQIIKK | 1055 |
| S1 | 1366 | GGD | 1368 |
| S2 | 1370 | GEE | 1372 |
| S3 | 1368 | GED | 1370 |
| S4 | 1056 | G-- | 1056 |

The alignment demonstrates that amino acid sequences and amino acid residues that are homologous to a reference Cas9 amino acid sequence or amino acid residue can be identified across Cas9 sequence variants, including, but not limited to Cas9 sequences from different species, by identifying the amino acid sequence or residue that aligns with the reference sequence or the reference residue using alignment programs and algorithms known in the art. This disclosure provides Cas9 variants in which one or more of the amino acid residues identified by an asterisk in SEQ ID NOs: 11-14 (e.g., S1, S2, S3, and S4, respectively) are mutated as described herein. The residues D10 and H840 in Cas9 of SEQ ID NO: 1 that correspond to the residues identified in SEQ ID NOs: 11-14 by an asterisk are referred to herein as “homologous” or “corresponding” residues. Such homologous residues can be identified by sequence alignment, e.g., as described above, and by identifying the sequence or residue that aligns with the reference sequence or residue. Similarly, mutations in Cas9 sequences that correspond to mutations identified in SEQ ID NO: 1 herein, e.g., mutations of residues 10, and 840 in SEQ ID NO: 1, are referred to herein as “homologous” or “corresponding” mutations. For example, the mutations corresponding to the D10A mutation in SEQ ID NO: 1 or S1 (SEQ ID NO: 11) for the four aligned sequences above are Dl 1A for S2, D10A for S3, and D13A for S4; the corresponding mutations for H840A in SEQ ID NO: 1 or S1 (SEQ ID NO: 11) are H850A for S2, H842A for S3, and H560A for S4.

A total of 250 Cas9 sequences (SEQ ID NOs: 11-260) from different species are provided. Amino acid residues homologous to residues 10, and 840 of SEQ ID NO: 1 may be identified in the same manner as outlined above. All of these Cas9 sequences may be used in accordance with the present disclosure.

| | |
|---|---|
| WP_010922251.1 | type II CRISPR RNA-guided endonuclease Cas9 [Streptococcus pyogenes] SEQ ID NO: 11 |
| WP_039695303.1 | type II CRISPR RNA-guided endonuclease Cas9 [Streptococcus gallolyticus] SEQ ID NO: 12 |
| WP_045635197.1 | type II CRISPR RNA-guided endonuclease Cas9 [Streptococcus mitis] SEQ ID NO: 13 |
| 5AXW_A | Cas9, Chain A, Crystal Structure [Staphylococcus Aureus] SEQ ID NO: 14 |
| WP_009880683.1 | type II CRISPR RNA-guided endonuclease Cas9 [Streptococcus pyogenes] SEQ ID NO: 15 |
| WP_010922251.1 | type II CRISPR RNA-guided endonuclease Cas9 [Streptococcus pyogenes] SEQ ID NO: 16 |
| WP_011054416.1 | type II CRISPR RNA-guided endonuclease Cas9 [Streptococcus pyogenes] SEQ ID NO: 17 |
| WP_011284745.1 | type II CRISPR RNA-guided endonuclease Cas9 [Streptococcus pyogenes] SEQ ID NO: 18 |
| WP_011285506.1 | type II CRISPR RNA-guided endonuclease Cas9 [Streptococcus pyogenes] SEQ ID NO: 19 |
| WP_011527619.1 | type II CRISPR RNA-guided endonuclease Cas9 [Streptococcus pyogenes] SEQ ID NO: 20 |
| WP_012560673.1 | type II CRISPR RNA-guided endonuclease Cas9 [Streptococcus pyogenes] SEQ ID NO: 21 |
| WP_014407541.1 | type II CRISPR RNA-guided endonuclease Cas9 [Streptococcus pyogenes] SEQ ID NO: 22 |
| WP_020905136.1 | type II CRISPR RNA-guided endonuclease Cas9 [Streptococcus pyogenes] SEQ ID NO: 23 |
| WP_023080005.1 | type II CRISPR RNA-guided endonuclease Cas9 [Streptococcus pyogenes] SEQ ID NO: 24 |
| WP_023610282.1 | type II CRISPR RNA-guided endonuclease Cas9 [Streptococcus pyogenes] SEQ ID NO: 25 |
| WP_030125963.1 | type II CRISPR RNA-guided endonuclease Cas9 [Streptococcus pyogenes] SEQ ID NO: 26 |
| WP_030126706.1 | type II CRISPR RNA-guided endonuclease Cas9 [Streptococcus pyogenes] SEQ ID NO: 27 |
| WP_031488318.1 | type II CRISPR RNA-guided endonuclease Cas9 [Streptococcus pyogenes] SEQ ID NO: 28 |
| WP_032460140.1 | type II CRISPR RNA-guided endonuclease Cas9 [Streptococcus pyogenes] SEQ ID NO: 29 |
| WP_032461047.1 | type II CRISPR RNA-guided endonuclease Cas9 [Streptococcus pyogenes] SEQ ID NO: 30 |
| WP_032462016.1 | type II CRISPR RNA-guided endonuclease Cas9 [Streptococcus pyogenes] SEQ ID NO: 31 |
| WP_032462936.1 | type II CRISPR RNA-guided endonuclease Cas9 [Streptococcus pyogenes] SEQ ID NO: 32 |
| WP_032464890.1 | type II CRISPR RNA-guided endonuclease Cas9 [Streptococcus pyogenes] SEQ ID NO: 33 |
| WP_033888930.1 | type II CRISPR RNA-guided endonuclease Cas9 [Streptococcus pyogenes] SEQ ID NO: 34 |
| WP_038431314.1 | type II CRISPR RNA-guided endonuclease Cas9 [Streptococcus pyogenes] SEQ ID NO: 35 |
| WP_038432938.1 | type II CRISPR RNA-guided endonuclease Cas9 [Streptococcus pyogenes] SEQ ID NO: 36 |
| WP_038434062.1 | type II CRISPR RNA-guided endonuclease Cas9 [Streptococcus pyogenes] SEQ ID NO: 37 |
| BAQ51233.1 | CRISPR-associated protein, Csn1 family [Streptococcus pyogenes] SEQ ID NO: 38 |
| KGE60162.1 | hypothetical protein MGAS2111_0903 [Streptococcus pyogenes MGAS2111] SEQ ID NO: 39 |
| KGE60856.1 | CRISPR-associated endonuclease protein [Streptococcus pyogenes SS1447] SEQ ID NO: 40 |
| WP_002989955.1 | MULTISPECIES: type II CRISPR RNA-guided endonuclease Cas9 [Streptococcus] SEQ ID NO: 41 |
| WP_003030002.1 | MULTISPECIES: type II CRISPR RNA-guided endonuclease Cas9 [Streptococcus] SEQ ID NO: 42 |
| WP_003065552.1 | MULTISPECIES: type II CRISPR RNA-guided endonuclease Cas9 [Streptococcus] SEQ ID NO: 43 |
| WP_001040076.1 | type II CRISPR RNA-guided endonuclease Cas9 [Streptococcus agalactiae] SEQ ID NO: 44 |
| WP_001040078.1 | type II CRISPR RNA-guided endonuclease Cas9 [Streptococcus agalactiae] SEQ ID NO: 45 |
| WP_001040080.1 | type II CRISPR RNA-guided endonuclease Cas9 [Streptococcus agalactiae] SEQ ID NO: 46 |
| WP_001040081.1 | type II CRISPR RNA-guided endonuclease Cas9 [Streptococcus agalactiae] SEQ ID NO: 47 |
| WP_001040083.1 | type II CRISPR RNA-guided endonuclease Cas9 [Streptococcus agalactiae] SEQ ID NO: 48 |
| WP_001040085.1 | type II CRISPR RNA-guided endonuclease Cas9 [Streptococcus agalactiae] SEQ ID NO: 49 |
| WP_001040087.1 | type II CRISPR RNA-guided endonuclease Cas9 [Streptococcus agalactiae] SEQ ID NO: 50 |
| WP_001040088.1 | type II CRISPR RNA-guided endonuclease Cas9 [Streptococcus agalactiae] SEQ ID NO: 51 |
| WP_001040089.1 | type II CRISPR RNA-guided endonuclease Cas9 [Streptococcus agalactiae] SEQ ID NO: 52 |
| WP_001040090.1 | type II CRISPR RNA-guided endonuclease Cas9 [Streptococcus agalactiae] SEQ ID NO: 53 |
| WP_001040091.1 | type II CRISPR RNA-guided endonuclease Cas9 [Streptococcus agalactiae] SEQ ID NO: 54 |
| WP_001040092.1 | type II CRISPR RNA-guided endonuclease Cas9 [Streptococcus agalactiae] SEQ ID NO: 55 |
| WP_001040094.1 | type II CRISPR RNA-guided endonuclease Cas9 [Streptococcus agalactiae] SEQ ID NO: 56 |
| WP_001040095.1 | type II CRISPR RNA-guided endonuclease Cas9 [Streptococcus agalactiae] SEQ ID NO: 57 |
| WP_001040096.1 | type II CRISPR RNA-guided endonuclease Cas9 [Streptococcus agalactiae] SEQ ID NO: 58 |
| WP_001040097.1 | type II CRISPR RNA-guided endonuclease Cas9 [Streptococcus agalactiae] SEQ ID NO: 59 |
| WP_001040098.1 | type II CRISPR RNA-guided endonuclease Cas9 [Streptococcus agalactiae] SEQ ID NO: 60 |
| WP_001040099.1 | type II CRISPR RNA-guided endonuclease Cas9 [Streptococcus agalactiae] SEQ ID NO: 61 |
| WP_001040100.1 | type II CRISPR RNA-guided endonuclease Cas9 [Streptococcus agalactiae] SEQ ID NO: 62 |
| WP_001040104.1 | type II CRISPR RNA-guided endonuclease Cas9 [Streptococcus agalactiae] SEQ ID NO: 63 |
| WP_001040105.1 | type II CRISPR RNA-guided endonuclease Cas9 [Streptococcus agalactiae] SEQ ID NO: 64 |
| WP_001040106.1 | type II CRISPR RNA-guided endonuclease Cas9 [Streptococcus agalactiae] SEQ ID NO: 65 |
| WP_001040107.1 | type II CRISPR RNA-guided endonuclease Cas9 [Streptococcus agalactiae] SEQ ID NO: 66 |
| WP_001040108.1 | type II CRISPR RNA-guided endonuclease Cas9 [Streptococcus agalactiae] SEQ ID NO: 67 |
| WP_001040109.1 | type II CRISPR RNA-guided endonuclease Cas9 [Streptococcus agalactiae] SEQ ID NO: 68 |
| WP_001040110.1 | type II CRISPR RNA-guided endonuclease Cas9 [Streptococcus agalactiae] SEQ ID NO: 69 |
| WP_015058523.1 | type II CRISPR RNA-guided endonuclease Cas9 [Streptococcus agalactiae] SEQ ID NO: 70 |
| WP_017643650.1 | type II CRISPR RNA-guided endonuclease Cas9 [Streptococcus agalactiae] SEQ ID NO: 71 |
| WP_017647151.1 | type II CRISPR RNA-guided endonuclease Cas9 [Streptococcus agalactiae] SEQ ID NO: 72 |
| WP_017648376.1 | type II CRISPR RNA-guided endonuclease Cas9 [Streptococcus agalactiae] SEQ ID NO: 73 |
| WP_017649527.1 | type II CRISPR RNA-guided endonuclease Cas9 [Streptococcus agalactiae] SEQ ID NO: 74 |
| WP_017771611.1 | type II CRISPR RNA-guided endonuclease Cas9 [Streptococcus agalactiae] SEQ ID NO: 75 |
| WP_017771984.1 | type II CRISPR RNA-guided endonuclease Cas9 [Streptococcus agalactiae] SEQ ID NO: 76 |
| CFQ25032.1 | CRISPR-associated protein [Streptococcus agalactiae] SEQ ID NO: 77 |
| CFV16040.1 | CRISPR-associated protein [Streptococcus agalactiae] SEQ ID NO: 78 |
| KLJ37842.1 | CRISPR-associated protein Csn1 [Streptococcus agalactiae] SEQ ID NO: 79 |
| KLJ72361.1 | CRISPR-associated protein Csn1 [Streptococcus agalactiae] SEQ ID NO: 80 |
| KLL20707.1 | CRISPR-associated protein Csn1 [Streptococcus agalactiae] SEQ ID NO: 81 |
| KLL42645.1 | CRISPR-associated protein Csn1 [Streptococcus agalactiae] SEQ ID NO: 82 |
| WP_047207273.1 | type II CRISPR RNA-guided endonuclease Cas9 [Streptococcus agalactiae] SEQ ID NO: 83 |
| WP_047209694.1 | type II CRISPR RNA-guided endonuclease Cas9 [Streptococcus agalactiae] SEQ ID NO: 84 |
| WP_050198062.1 | type II CRISPR RNA-guided endonuclease Cas9 [Streptococcus agalactiae] SEQ ID NO: 85 |
| WP_050201642.1 | type II CRISPR RNA-guided endonuclease Cas9 [Streptococcus agalactiae] SEQ ID NO: 86 |
| WP_050204027.1 | type II CRISPR RNA-guided endonuclease Cas9 [Streptococcus agalactiae] SEQ ID NO: 87 |
| WP_050881965.1 | type II CRISPR RNA-guided endonuclease Cas9 [Streptococcus agalactiae] SEQ ID NO: 88 |
| WP_050886065.1 | type II CRISPR RNA-guided endonuclease Cas9 [Streptococcus agalactiae] SEQ ID NO: 89 |
| AHN30376.1 | CRISPR-associated protein Csn1 [Streptococcus agalactiae 138P] SEQ ID NO: 90 |
| EAO78426.1 | reticulocyte binding protein [Streptococcus agalactiae H36B] SEQ ID NO: 91 |
| CCW42055.1 | CRISPR-associated protein, SAG0894 family [Streptococcus agalactiae ILRI112] SEQ ID NO: 92 |
| WP_003041502.1 | type II CRISPR RNA-guided endonuclease Cas9 [Streptococcus anginosus] SEQ ID NO: 93 |
| WP_037593752.1 | type II CRISPR RNA-guided endonuclease Cas9 [Streptococcus anginosus] SEQ ID NO: 94 |
| WP_049516684.1 | CRISPR-associated protein Csn1 [Streptococcus anginosus] SEQ ID NO: 95 |
| GAD46167.1 | hypothetical protein ANG6_0662 [Streptococcus anginosus T5] SEQ ID NO: 96 |
| WP_018363470.1 | type II CRISPR RNA-guided endonuclease Cas9 [Streptococcus caballi] SEQ ID NO: 97 |
| WP_003043819.1 | type II CRISPR RNA-guided endonuclease Cas9 [Streptococcus canis] SEQ ID NO: 98 |
| WP_006269658.1 | type II CRISPR RNA-guided endonuclease Cas9 [Streptococcus constellatus] SEQ ID NO: 99 |
| WP_048800889.1 | type II CRISPR RNA-guided endonuclease Cas9 [Streptococcus constellatus] SEQ ID NO: 100 |
| WP_012767106.1 | type II CRISPR RNA-guided endonuclease Cas9 [Streptococcus dysgalactiae] SEQ ID NO: 101 |
| WP_014612333.1 | type II CRISPR RNA-guided endonuclease Cas9 [Streptococcus dysgalactiae] SEQ ID NO: 102 |
| WP_015017095.1 | type II CRISPR RNA-guided endonuclease Cas9 [Streptococcus dysgalactiae] SEQ ID NO: 103 |
| WP_015057649.1 | type II CRISPR RNA-guided endonuclease Cas9 [Streptococcus dysgalactiae] SEQ ID NO: 104 |
| WP_048327215.1 | type II CRISPR RNA-guided endonuclease Cas9 [Streptococcus dysgalactiae] SEQ ID NO: 105 |
| WP_049519324.1 | CRISPR-associated protein Csn1 [Streptococcus dysgalactiae] SEQ ID NO: 106 |
| WP_012515931.1 | type II CRISPR RNA-guided endonuclease Cas9 [Streptococcus equi] SEQ ID NO: 107 |
| WP_021320964.1 | type II CRISPR RNA-guided endonuclease Cas9 [Streptococcus equi] SEQ ID NO: 108 |
| WP_037581760.1 | type II CRISPR RNA-guided endonuclease Cas9 [Streptococcus equi] SEQ ID NO: 109 |
| WP_004232481.1 | type II CRISPR RNA-guided endonuclease Cas9 [Streptococcus equinus] SEQ ID NO: 110 |
| WP_009854540.1 | type II CRISPR RNA-guided endonuclease Cas9 [Streptococcus gallolyticus] SEQ ID NO: 111 |
| WP_012962174.1 | type II CRISPR RNA-guided endonuclease Cas9 [Streptococcus gallolyticus] SEQ ID NO: 112 |
| WP_039695303.1 | type II CRISPR RNA-guided endonuclease Cas9 [Streptococcus gallolyticus] SEQ ID NO: 113 |
| WP_014334983.1 | type II CRISPR RNA-guided endonuclease Cas9 [Streptococcus infantarius] SEQ ID NO: 114 |
| WP_003099269.1 | type II CRISPR RNA-guided endonuclease Cas9 [Streptococcus iniae] SEQ ID NO: 115 |
| AHY15608.1 | CRISPR-associated protein Csn1 [Streptococcus iniae] SEQ ID NO: 116 |
| AHY17476.1 | CRISPR-associated protein Csn1 [Streptococcus iniae] SEQ ID NO: 117 |
| ESR09100.1 | hypothetical protein IUSA1_08595 [Streptococcus iniae IUSA1] SEQ ID NO: 118 |
| AGM98575.1 | CRISPR-associated protein Cas9/Csn1, subtype II/NMEMI [Streptococcus iniae SF1] SEQ ID NO: 119 |
| ALF27331.1 | CRISPR-associated protein Csn1 [Streptococcus intermedius] SEQ ID NO: 120 |
| WP_018372492.1 | type II CRISPR RNA-guided endonuclease Cas9 [Streptococcus massiliensis] SEQ ID NO: 121 |
| WP_045618028.1 | type II CRISPR RNA-guided endonuclease Cas9 [Streptococcus mitis] SEQ ID NO: 122 |
| WP_045635197.1 | type II CRISPR RNA-guided endonuclease Cas9 [Streptococcus mitis] SEQ ID NO: 123 |
| WP_002263549.1 | type II CRISPR RNA-guided endonuclease Cas9 [Streptococcus mutans] SEQ ID NO: 124 |
| WP_002263887.1 | type II CRISPR RNA-guided endonuclease Cas9 [Streptococcus mutans] SEQ ID NO: 125 |
| WP_002264920.1 | type II CRISPR RNA-guided endonuclease Cas9 [Streptococcus mutans] SEQ ID NO: 126 |
| WP_002269043.1 | type II CRISPR RNA-guided endonuclease Cas9 [Streptococcus mutans] SEQ ID NO: 127 |
| WP_002269448.1 | type II CRISPR RNA-guided endonuclease Cas9 [Streptococcus mutans] SEQ ID NO: 128 |
| WP_002271977.1 | type II CRISPR RNA-guided endonuclease Cas9 [Streptococcus mutans] SEQ ID NO: 129 |
| WP_002272766.1 | type II CRISPR RNA-guided endonuclease Cas9 [Streptococcus mutans] SEQ ID NO: 130 |
| WP_002273241.1 | type II CRISPR RNA-guided endonuclease Cas9 [Streptococcus mutans] SEQ ID NO: 131 |
| WP_002275430.1 | type II CRISPR RNA-guided endonuclease Cas9 [Streptococcus mutans] SEQ ID NO: 132 |
| WP_002276448.1 | type II CRISPR RNA-guided endonuclease Cas9 [Streptococcus mutans] SEQ ID NO: 133 |
| WP_002277050.1 | type II CRISPR RNA-guided endonuclease Cas9 [Streptococcus mutans] SEQ ID NO: 134 |
| WP_002277364.1 | type II CRISPR RNA-guided endonuclease Cas9 [Streptococcus mutans] SEQ ID NO: 135 |
| WP_002279025.1 | type II CRISPR RNA-guided endonuclease Cas9 [Streptococcus mutans] SEQ ID NO: 136 |
| WP_002279859.1 | type II CRISPR RNA-guided endonuclease Cas9 [Streptococcus mutans] SEQ ID NO: 137 |
| WP_002280230.1 | type II CRISPR RNA-guided endonuclease Cas9 [Streptococcus mutans] SEQ ID NO: 138 |
| WP_002281696.1 | type II CRISPR RNA-guided endonuclease Cas9 [Streptococcus mutans] SEQ ID NO: 139 |
| WP_002282247.1 | type II CRISPR RNA-guided endonuclease Cas9 [Streptococcus mutans] SEQ ID NO: 140 |
| WP_002282906.1 | type II CRISPR RNA-guided endonuclease Cas9 [Streptococcus mutans] SEQ ID NO: 141 |
| WP_002283846.1 | type II CRISPR RNA-guided endonuclease Cas9 [Streptococcus mutans] SEQ ID NO: 142 |
| WP_002287255.1 | type II CRISPR RNA-guided endonuclease Cas9 [Streptococcus mutans] SEQ ID NO: 143 |
| WP_002288990.1 | type II CRISPR RNA-guided endonuclease Cas9 [Streptococcus mutans] SEQ ID NO: 144 |
| WP_002289641.1 | type II CRISPR RNA-guided endonuclease Cas9 [Streptococcus mutans] SEQ ID NO: 145 |
| WP_002290427.1 | type II CRISPR RNA-guided endonuclease Cas9 [Streptococcus mutans] SEQ ID NO: 146 |
| WP_002295753.1 | type II CRISPR RNA-guided endonuclease Cas9 [Streptococcus mutans] SEQ ID NO: 147 |
| WP_002296423.1 | type II CRISPR RNA-guided endonuclease Cas9 [Streptococcus mutans] SEQ ID NO: 148 |
| WP_002304487.1 | type II CRISPR RNA-guided endonuclease Cas9 [Streptococcus mutans] SEQ ID NO: 149 |
| WP_002305844.1 | type II CRISPR RNA-guided endonuclease Cas9 [Streptococcus mutans] SEQ ID NO: 150 |
| WP_002307203.1 | type II CRISPR RNA-guided endonuclease Cas9 [Streptococcus mutans] SEQ ID NO: 151 |
| WP_002310390.1 | type II CRISPR RNA-guided endonuclease Cas9 [Streptococcus mutans] SEQ ID NO: 152 |
| WP_002352408.1 | type II CRISPR RNA-guided endonuclease Cas9 [Streptococcus mutans] SEQ ID NO: 153 |
| WP_012997688.1 | type II CRISPR RNA-guided endonuclease Cas9 [Streptococcus mutans] SEQ ID NO: 154 |
| WP_014677909.1 | type II CRISPR RNA-guided endonuclease Cas9 [Streptococcus mutans] SEQ ID NO: 155 |
| WP_019312892.1 | type II CRISPR RNA-guided endonuclease Cas9 [Streptococcus mutans] SEQ ID NO: 156 |
| WP_019313659.1 | type II CRISPR RNA-guided endonuclease Cas9 [Streptococcus mutans] SEQ ID NO: 157 |
| WP_019314093.1 | type II CRISPR RNA-guided endonuclease Cas9 [Streptococcus mutans] SEQ ID NO: 158 |
| WP_019315370.1 | type II CRISPR RNA-guided endonuclease Cas9 [Streptococcus mutans] SEQ ID NO: 159 |
| WP_019803776.1 | type II CRISPR RNA-guided endonuclease Cas9 [Streptococcus mutans] SEQ ID NO: 160 |
| WP_019805234.1 | type II CRISPR RNA-guided endonuclease Cas9 [Streptococcus mutans] SEQ ID NO: 161 |
| WP_024783594.1 | type II CRISPR RNA-guided endonuclease Cas9 [Streptococcus mutans] SEQ ID NO: 162 |
| WP_024784288.1 | type II CRISPR RNA-guided endonuclease Cas9 [Streptococcus mutans] SEQ ID NO: 163 |
| WP_024784666.1 | type II CRISPR RNA-guided endonuclease Cas9 [Streptococcus mutans] SEQ ID NO: 164 |
| WP_024784894.1 | type II CRISPR RNA-guided endonuclease Cas9 [Streptococcus mutans] SEQ ID NO: 165 |
| WP_024786433.1 | type II CRISPR RNA-guided endonuclease Cas9 [Streptococcus mutans] SEQ ID NO: 166 |
| WP_049473442.1 | CRISPR-associated protein Csn1 [Streptococcus mutans] SEQ ID NO: 167 |
| WP_049474547.1 | CRISPR-associated protein Csn1 [Streptococcus mutans] SEQ ID NO: 168 |
| EMC03581.1 | hypothetical protein SMU69_09359 [Streptococcus mutans NLML4] SEQ ID NO: 169 |
| WP_000428612.1 | type II CRISPR RNA-guided endonuclease Cas9 [Streptococcus oralis] SEQ ID NO: 170 |
| WP_000428613.1 | type II CRISPR RNA-guided endonuclease Cas9 [Streptococcus oralis] SEQ ID NO: 171 |
| WP_049523028.1 | CRISPR-associated protein Csn1 [Streptococcus parasanguinis] SEQ ID NO: 172 |
| WP_003107102.1 | type II CRISPR RNA-guided endonuclease Cas9 [Streptococcus parauberis] SEQ ID NO: 173 |
| WP_054279288.1 | type II CRISPR RNA-guided endonuclease Cas9 [Streptococcus phocae] SEQ ID NO: 174 |
| WP_049531101.1 | CRISPR-associated protein Csn1 [Streptococcus pseudopneumoniae] SEQ ID NO: 175 |
| WP_049538452.1 | CRISPR-associated protein Csn1 [Streptococcus pseudopneumoniae] SEQ ID NO: 176 |
| WP_049549711.1 | CRISPR-associated protein Csn1 [Streptococcus pseudopneumoniae] SEQ ID NO: 177 |
| WP_007896501.1 | type II CRISPR RNA-guided endonuclease Cas9 [Streptococcus pseudoporcinus] SEQ ID NO: 178 |
| EFR44625.1 | CRISPR-associated protein, Csn1 family [Streptococcus pseudoporcinus SPIN 20026] SEQ ID NO: 179 |
| WP_002897477.1 | type II CRISPR RNA-guided endonuclease Cas9 [Streptococcus sanguinis] SEQ ID NO: 180 |
| WP_002906454.1 | type II CRISPR RNA-guided endonuclease Cas9 [Streptococcus sanguinis] SEQ ID NO: 181 |
| WP_009729476.1 | type II CRISPR RNA-guided endonuclease Cas9 [Streptococcussp. F0441] SEQ ID NO: 182 |
| CQR24647.1 | CRISPR-associated protein [Streptococcus sp. FF10] SEQ ID NO: 183 |
| WP_000066813.1 | type II CRISPR RNA-guided endonuclease Cas9 [Streptococcus sp. M334] SEQ ID NO: 184 |
| WP_009754323.1 | type II CRISPR RNA-guided endonuclease Cas9 [Streptococcus sp. taxon 056] SEQ ID NO: 185 |
| WP_044674937.1 | type II CRISPR RNA-guided endonuclease Cas9 [Streptococcus suis] SEQ ID NO: 186 |
| WP_044676715.1 | type II CRISPR RNA-guided endonuclease Cas9 [Streptococcus suis] SEQ ID NO: 187 |
| WP_044680361.1 | type II CRISPR RNA-guided endonuclease Cas9 [Streptococcus suis] SEQ ID NO: 188 |
| WP_044681799.1 | type II CRISPR RNA-guided endonuclease Cas9 [Streptococcus suis] SEQ ID NO: 189 |
| WP_049533112.1 | CRISPR-associated protein Csn1 [Streptococcus suis] SEQ ID NO: 190 |
| WP_029090905.1 | type II CRISPR RNA-guided endonuclease Cas9 [Brochothrix thermosphacta] SEQ ID NO: 191 |
| WP_006506696.1 | type II CRISPR RNA-guided endonuclease Cas9 [Catenibacterium mitsuokai] SEQ ID NO: 192 |
| AIT42264.1 | Cas9hc:NLS:HA [Cloning vector pYB196] SEQ ID NO: 193 |
| WP_034440723.1 | type II CRISPR endonuclease Cas9 [Clostridiales bacterium S5-A11] SEQ ID NO: 194 |
| AKQ21048.1 | Cas9 [CRISPR-mediated gene targeting vector p(bhsp68-Cas9)] SEQ ID NO: 195 |
| WP_004636532.1 | type II CRISPR RNA-guided endonuclease Cas9 [Dolosigranulum pigrum] SEQ ID NO: 196 |
| WP_002364836.1 | MULTISPECIES: type II CRISPR RNA-guided endonuclease Cas9 [Enterococcus] SEQ ID NO: 197 |
| WP_016631044.1 | MULTISPECIES: type II CRISPR RNA-guided endonuclease Cas9 [Enterococcus] SEQ ID NO: 198 |
| EMS75795.1 | hypothetical protein H318_06676 [Enterococcus durans IPLA 655] SEQ ID NO: 199 |
| WP_002373311.1 | type II CRISPR RNA-guided endonuclease Cas9 [Enterococcus faecalis] SEQ ID NO: 200 |
| WP_002378009.1 | type II CRISPR RNA-guided endonuclease Cas9 [Enterococcus faecalis] SEQ ID NO: 201 |
| WP_002407324.1 | type II CRISPR RNA-guided endonuclease Cas9 [Enterococcus faecalis] SEQ ID NO: 202 |
| WP_002413717.1 | type II CRISPR RNA-guided endonuclease Cas9 [Enterococcus faecalis] SEQ ID NO: 203 |
| WP_010775580.1 | type II CRISPR RNA-guided endonuclease Cas9 [Enterococcus faecalis] SEQ ID NO: 204 |
| WP_010818269.1 | type II CRISPR RNA-guided endonuclease Cas9 [Enterococcus faecalis] SEQ ID NO: 205 |
| WP_010824395.1 | type II CRISPR RNA-guided endonuclease Cas9 [Enterococcus faecalis] SEQ ID NO: 206 |
| WP_016622645.1 | type II CRISPR RNA-guided endonuclease Cas9 [Enterococcus faecalis] SEQ ID NO: 207 |
| WP_033624816.1 | type II CRISPR RNA-guided endonuclease Cas9 [Enterococcus faecalis] SEQ ID NO: 208 |
| WP_033625576.1 | type II CRISPR RNA-guided endonuclease Cas9 [Enterococcus faecalis] SEQ ID NO: 209 |
| WP_033789179.1 | type II CRISPR RNA-guided endonuclease Cas9 [Enterococcus faecalis] SEQ ID NO: 210 |
| WP_002310644.1 | type II CRISPR RNA-guided endonuclease Cas9 [Enterococcus faecium] SEQ ID NO: 211 |
| WP_002312694.1 | type II CRISPR RNA-guided endonuclease Cas9 [Enterococcus faecium] SEQ ID NO: 212 |
| WP_002314015.1 | type II CRISPR RNA-guided endonuclease Cas9 [Enterococcus faecium] SEQ ID NO: 213 |
| WP_002320716.1 | type II CRISPR RNA-guided endonuclease Cas9 [Enterococcus faecium] SEQ ID NO: 214 |
| WP_002330729.1 | type II CRISPR RNA-guided endonuclease Cas9 [Enterococcus faecium] SEQ ID NO: 215 |
| WP_002335161.1 | type II CRISPR RNA-guided endonuclease Cas9 [Enterococcus faecium] SEQ ID NO: 216 |
| WP_002345439.1 | type II CRISPR RNA-guided endonuclease Cas9 [Enterococcus faecium] SEQ ID NO: 217 |
| WP_034867970.1 | type II CRISPR RNA-guided endonuclease Cas9 [Enterococcus faecium] SEQ ID NO: 218 |
| WP_047937432.1 | type II CRISPR RNA-guided endonuclease Cas9 [Enterococcus faecium] SEQ ID NO: 219 |
| WP_010720994.1 | type II CRISPR RNA-guided endonuclease Cas9 [Enterococcus hirae] SEQ ID NO: 220 |
| WP_010737004.1 | type II CRISPR RNA-guided endonuclease Cas9 [Enterococcus hirae] SEQ ID NO: 221 |
| WP_034700478.1 | type II CRISPR RNA-guided endonuclease Cas9 [Enterococcus hirae] SEQ ID NO: 222 |
| WP_007209003.1 | type II CRISPR RNA-guided endonuclease Cas9 [Enterococcus italicus] SEQ ID NO: 223 |
| WP_023519017.1 | type II CRISPR RNA-guided endonuclease Cas9 [Enterococcus mundtii] SEQ ID NO: 224 |
| WP_010770040.1 | type II CRISPR RNA-guided endonuclease Cas9 [Enterococcus phoeniculicola] SEQ ID NO: 225 |
| WP_048604708.1 | type II CRISPR RNA-guided endonuclease Cas9 [Enterococcus sp. AM1] SEQ ID NO: 226 |
| WP_010750235.1 | type II CRISPR RNA-guided endonuclease Cas9 [Enterococcus villorum] SEQ ID NO: 227 |
| AII16583.1 | Cas9 endonuclease [Expression vector pCas9] SEQ ID NO: 228 |
| WP_029073316.1 | type II CRISPR RNA-guided endonuclease Cas9 [Kandleria vitulina] SEQ ID NO: 229 |
| WP_031589969.1 | type II CRISPR RNA-guided endonuclease Cas9 [Kandleria vitulina] SEQ ID NO: 230 |
| KDA45870.1 | CRISPR-associated protein Cas9/Csn1, subtype II/NMEMI [Lactobacillus animalis] SEQ ID NO: 231 |
| WP_039099354.1 | type II CRISPR RNA-guided endonuclease Cas9 [Lactobacillus curvatus] SEQ ID NO: 232 |
| AKP02966.1 | hypothetical protein ABB45_04605 [Lactobacillus farciminis] SEQ ID NO: 233 |
| WP_010991369.1 | type II CRISPR RNA-guided endonuclease Cas9 [Listeria innocua] SEQ ID NO: 234 |
| WP_033838504.1 | type II CRISPR RNA-guided endonuclease Cas9 [Listeria innocua] SEQ ID NO: 235 |
| EHN60060.1 | CRISPR-associated protein, Csn1 family [Listeria innocua ATCC 33091] SEQ ID NO: 236 |
| EFR89594.1 | crispr-associated protein, Csn1 family [Listeria innocua FSL S4-378] SEQ ID NO: 237 |
| WP_038409211.1 | type II CRISPR RNA-guided endonuclease Cas9 [Listeria ivanovii] SEQ ID NO: 238 |
| EFR95520.1 | crispr-associated protein Csn1 [Listeria ivanovii FSL F6-596] SEQ ID NO: 239 |
| WP_003723650.1 | type II CRISPR RNA-guided endonuclease Cas9 [Listeria monocytogenes] SEQ ID NO: 240 |
| WP_003727705.1 | type II CRISPR RNA-guided endonuclease Cas9 [Listeria monocytogenes] SEQ ID NO: 241 |
| WP_003730785.1 | type II CRISPR RNA-guided endonuclease Cas9 [Listeria monocytogenes] SEQ ID NO: 242 |
| WP_003733029.1 | type II CRISPR RNA-guided endonuclease Cas9 [Listeria monocytogenes] SEQ ID NO: 243 |
| WP_003739838.1 | type II CRISPR RNA-guided endonuclease Cas9 [Listeria monocytogenes] SEQ ID NO: 244 |
| WP_014601172.1 | type II CRISPR RNA-guided endonuclease Cas9 [Listeria monocytogenes] SEQ ID NO: 245 |
| WP_023548323.1 | type II CRISPR RNA-guided endonuclease Cas9 [Listeria monocytogenes] SEQ ID NO: 246 |
| WP_031665337.1 | type II CRISPR RNA-guided endonuclease Cas9 [Listeria monocytogenes] SEQ ID NO: 247 |
| WP_031669209.1 | type II CRISPR RNA-guided endonuclease Cas9 [Listeria monocytogenes] SEQ ID NO: 248 |
| WP_033920898.1 | type II CRISPR RNA-guided endonuclease Cas9 [Listeria monocytogenes] SEQ ID NO: 249 |
| AKI42028.1 | CRISPR-associated protein [Listeria monocytogenes] SEQ ID NO: 250 |
| AKI50529.1 | CRISPR-associated protein [Listeria monocytogenes] SEQ ID NO: 251 |
| EFR83390.1 | crispr-associated protein Csn1 [Listeria monocytogenes FSL F2-208] SEQ ID NO: 252 |
| WP_046323366.1 | type II CRISPR RNA-guided endonuclease Cas9 [Listeria seeligeri] SEQ ID NO: 253 |
| AKE81011.1 | Cas9 [Plant multiplex genome editing vector pYLCRISPR/Cas9Pubi-H] SEQ ID NO: 254 |
| CUO82355.1 | Uncharacterized protein conserved in bacteria [Roseburia hominis] SEQ ID NO: 255 |
| WP_033162887.1 | type II CRISPR RNA-guided endonuclease Cas9 [Sharpea azabuensis] SEQ ID NO: 256 |
| AGZ01981.1 | Cas9 endonuclease [synthetic construct] SEQ ID NO: 257 |
| AKA60242.1 | nuclease deficient Cas9 [synthetic construct] SEQ ID NO: 258 |
| AKS40380.1 | Cas9 [Synthetic plasmid pFC330] SEQ ID NO: 259 |
| 4UN5_B | Cas9, Chain B, Crystal Structure SEQ ID NO: 260 |

Non-limiting examples of suitable deaminase domains are provided.

Human AID
(SEQ ID NO: 303)

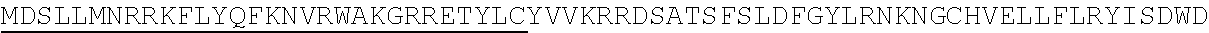

LDPGRCYRVTWFTSWSPCYDCARHVADFLRGNPNLSLRIFTARLYFCEDRKAEPEGLRRLHRAGVQIAIMT
FKDYFYCWNTFVENHERTFKAWEGLHENSVRLSRQLRRILLPLYEVDDLRDAFRTLGL
(underline: nuclear localization signal; double underline: nuclear export signal)
Mouse AID
(SEQ ID NO: 271)
MDSLLMKQKKFLYHFKNVRWAKGRHETYLCYVVKRRDSATSCSLDFGHLRNKSGCHVELLFLRYISDWD
LDPGRCYRVTWFTSWSPCYDCARHVAEFLRWNPNLSLRIFTARLYFCEDRKAEPEGLRRLHRAGVQIGIMT
FKDYFYCWNTFVENRERTFKAWEGLHENSVRLTRQLRRILLPLYEVDDLRDAFRMLGE
(underline: nuclear localization signal; double underline: nuclear export signal)
Dog AID
(SEQ ID NO: 272)

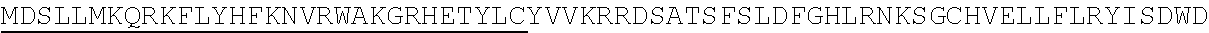

LDPGRCYRVTWFTSWSPCYDCARHVADFLRGYPNLSLRIFAARLYFCEDRKAEPEGLRRLHRAGVQIAIMT
FKDYFYCWNTFVENREKTFKAWEGLHENSVRLSRQLRRILLPLYEVDDLRDAFRTLGL
(underline: nuclear localization signal; double underline: nuclear export signal)
Bovine AID
(SEQ ID NO: 273)

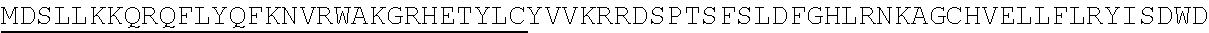

LDPGRCYRVTWFTSWSPCYDCARHVADFLRGYPNLSLRIFTARLYFCDKERKAEPEGLRRLHRAGVQIAIM

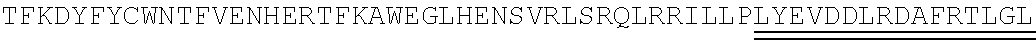

(underline: nuclear localization signal; double underline: nuclear export signal)
Rat AID
(SEQ ID NO: 2483)
MAVGSKPKAALVGPHWERERIWCFLCSTGLGTQQTGQTSRWLRPAATQDPVSPPRSLLMKQRKFLYHFK
NVRWAKGRHETYLCYVVKRRDSATSFSLDFGYLRNKSGCHVELLFLRYISDWDLDPGRCYRVTWFTSWS
PCYDCARHVADFLRGNPNLSLRIFTARLTGWGALPAGLMSPARPSDYFYCWNTFVENHERTFKAWEGLHE
NSVRLSRRLRRILLPLYEVDDLRDAFRTLGL
Mouse APOBEC-3
(SEQ ID NO: 274)
MGPFCLGCSHRKCYSPIRNLISQETFKFHFKNLGYAKGRKDTFLCYEVTRKDCDSPVSLHHGVFKNKDNIH
AEICFLYWFHDKVLKVLSPREEFKITWYMSWSPCFECAEQIVRFLATHHNLSLDIFSSRLYNVQDPETQQNLCR
LVQEGAQVAAMDLYEFKKCWKKFVDNGGRRFRPWKRLLTNFRYQDSKLQEILRPCYIPVPSSSSSTLSNIC
LTKGLPETRFCVEGRRMDPLSEEEFYSQFYNQRVKHLCYYHRMKPYLCYQLEQFNGQAPLKGCLLSEKGK
QHAEILFLDKIRSMELSQVTITCYLTWSPCPNCAWQLAAFKRDRPDLILHIYTSRLYFHWKRPFQKGLCSLWQ
SGILVDVMDLPQFTDCWTNFVNPKRPFWPWKGLEIISRRTQRRLRRIKESWGLQDLVNDFGNLQLGPPMS
(italic: nucleic acid editing domain)
Rat APOBEC-3
(SEQ ID NO: 275)
MGPFCLGCSHRKCYSPIRNLISQETFKFHFKNLRYAIDRKDTFLCYEVTRKDCDSPVSLHHGVFKNKDNIHA
EICFLYWFHDKVLKVLSPREEFKITWYMSWSPCFECAEQVLRFLATHHNLSLDIFSSRLYNIRDPENQQNLCRL
VQEGAQVAAMDLYEFKKCWKKFVDNGGRRFRPWKKLLTNFRYQDSKLQEILRPCYIPVPSSSSSTLSNICL
TKGLPETRFCVERRRVHLLSEEEFYSQFYNQRVKHLCYYHGVKPYLCYQLEQFNGQAPLKGCLLSEKGKQ
HAEILFLDKIRSMELSQVIITCYLTWSPCPNCAWQLAAFKRDRPDLILHIYTSRLYFHWKRPFQKGLCSLWQSG
ILVDVMDLPQFTDCWTNFVNPKRPFWPWKGLEISRRTQRRLHRIKESWGLQDLVNDFGNLQLGPPMS
(italic: nucleic acid editing domain)
Rhesus macaque APOBEC-3G
(SEQ ID NO: 276)

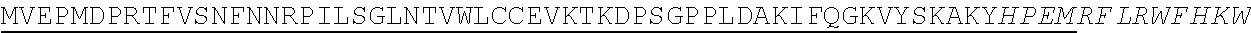

RQLHHDQEYKVTWYVSWSPCTRCANSVATFLAKDPKVTLTIFVARLYYFWKPDYQQALRILCQKRGGPHAT
MKIMNYNEFQDCWNKFVDGRGKPFKPRNNLPKHYTLLQATLGELLRHLMDPGTFTSNFNNKPWVSGQHE
TYLCYKVERLHNDTWVPLNQHRGFLRNQAPNIHGFPKGRHAELCFLDLIPFWKLDGQQYRVTCFTSWSPCFS
CAQEMAKFISNNEHVSLCIFAARIYDDQGRYQEGLRALHRDGAKIAMMNYSEFEYCWDTFVDRQGRPFQP
WDGLDEHSQALSGRLRAI (italic: nucleic acid editing domain; underline: cytoplasmic
localization signal)
Chimpanzee APOBEC-3G
(SEQ ID NO: 277)
MKPHERNPVERMYQDTESDNFYNRPILSHRNTVWLCYEVKTKGPSRPPLDAKIERGQVYSKLKYHPEMRF
FHWFSKWRKLHRDQEYEVTWYISWSPCTKCTRDVATFLAEDPKVTLTIFVARLYYFWDPDYQEALRSLCQKR
DGPRATMKIMNYDEFQHCWSKFVYSQRELFEPWNNLPKYYILLHIMLGEILRHSMDPPTFTSNENNELWVR
GRHETYLCYEVERLHNDTWVLLNQRRGFLCNQAPHKHGFLEGRHAELCFLDVIPFWKLDLHQDYRVTCFTS
WSPCFSCAQEMAKFISNNKHVSLCIFAARIYDDQGRCQEGLRTLAKAGAKISIMTYSEFKHCWDTFVDHQG
CPFQPWDGLEEHSQALSGRLRAILQNQGN (italic: nucleic acid editing domain; underline:
cytoplasmic localization signal)
Green monkey APOBEC-3G
(SEQ ID NO: 278)
MNPQIRNMVEQMEPDIFVYYENNRPILSGRNTVWLCYEVKTKDPSGPPLDANIFQGKLYPEAKDHPEMKEL
HWFRKWRQLHRDQEYEVTWYVSWSPCTRCANSVATFLAEDPKVTLTIFVARLYYFWKPDYQQALRILCQER
GGPHATMKIMNYNEFQHCWNEFVDGQGKPFKPRKNLPKHYTLLHATLGELLRHVMDPGTFTSNFNNKPW
VSGQRETYLCYKVERSHNDTWVLLNQHRGFLRNQAPDRHGFPKGRHAELCFLDLIPFWKLDDQQYRVTCFT
SWSPCFSCAQKMAKFISNNKHVSLCIFAARIYDDQGRCQEGLRTLHRDGAKIAVMNYSEFEYCWDTFVDR
QGRPFQPWDGLDEHSQALSGRLRAI (italic: nucleic acid editing domain; underline:
cytoplasmic localization signal)
Human APOBEC-3G
(SEQ ID NO: 279)

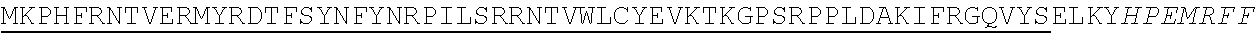

HWFSKWRKLHRDQEYEVTWYISWSPCTKCTRDMATFLAEDPKVTLTIFVARLYYFWDPDYQEALRSLCQKR
DGPRATMKIMNYDEFQHCWSKFVYSQRELFEPWNNLPKYYILLHIMLGEILRHSMDPPTFTFNFNNEPWVR
GRHETYLCYEVERMHNDTWVLLNQRRGFLCNQAPHKHGFLEGRHAELCFLDVIPFWKLDLDQDYRVTCFTS
WSPCFSCAQEMAKFISKNKHVSLCIFTARIYDDQGRCQEGLRTLAEAGAKISIMTYSEFKHCWDTFVDHQG
CPFQPWDGLDEHSQDLSGRLRAILQNQEN (italic: nucleic acid editing domain; underline:
cytoplasmic localization signal)
Human APOBEC-3F
(SEQ ID NO: 280)
MKPHFRNTVERMYRDTFSYNFYNRPILSRRNTVWLCYEVKTKGPSRPRLDAKIFRGQVYSQPEHHAEMCFL
SWFCGNQLPAYKCFQITWFVSWTPCPDCVAKLAEFLAEHPNVTLTISAARLYYYWERDYRRALCRLSQAGA
RVKIMDDEEFAYCWENFVYSEGQPFMPWYKFDDNYAFLHRTLKEILRNPMEAMYPHIFYFHFKNLRKAY
GRNESWLCFTMEVVKHHSPVSWKRGVFRNQVDPETHCHAERCFLSWFCDDILSPNTNYEVTWYTSWSPCPE
CAGEVAEFLARHSNVNLTIFTARLYYFWDTDYQEGLRSLSQEGASVEIMGYKDFKYCWENFVYNDDEPFK
PWKGLKYNFLFLDSKLQEILE (italic: nucleic acid editing domain)
Human APOBEC-3B
(SEQ ID NO: 281)
MNPQIRNPMERMYRDTFYDNFENEPILYGRSYTWLCYEVKIKRGRSNLLWDTGVFRGQVYFKPQYHAEM
CFLSWFCGNQLPAYKCFQITWFVSWTPCPDCVAKLAEFLSEHPNVTLTISAARLYYYWERDYRRALCRLSQA
GARVTIMDYEEFAYCWENFVYNEGQQFMPWYKEDENYAFLHRTLKEILRYLMDPDTFTFNENNDPLVLRR
RQTYLCYEVERLDNGTWVLMDQHMGFLCNEAKNLLCGFYGRHAELRFLDLVPSLQLDPAQIYRVTWFISWS
PCFSWGCAGEVRAFLQENTHVRLRIFAARIYDYDPLYKEALQMLRDAGAQVSIMTYDEFEYCWDTFVYRQ
GCPFQPWDGLEEHSQALSGRLRAILQNQGN (italic: nucleic acid editing domain)
Rat APOBEC-3B:
(SEQ ID NO: 2484)
MQPQGLGPNAGMGPVCLGCSHRRPYSPIRNPLKKLYQQTFYFHFKNVRYAWGRKNNFLCYEVNGMDCAL
PVPLRQGVFRKQGHIHAELCFIYWFHDKVLRVLSPMEEFKVTWYMSWSPCSKCAEQVARFLAAHRNLSLA
IFSSRLYYYLRNPNYQQKLCRLIQEGVHVAAMDLPEFKKCWNKFVDNDGQPFRPWMRLRINESFYDCKLQ
EIFSRMNLLREDVFYLQFNNSHRVKPVQNRYYRRKSYLCYQLERANGQEPLKGYLLYKKGEQHVEILFLE
KMRSMELSQVRITCYLTWSPCPNCARQLAAFKKDHPDLILRIYTSRLYFYWRKKFQKGLCTLWRSGIHVD
VMDLPQFADCWTNFVNPQRPFRPWNELEKNSWRIQRRLRRIKESWGL
Bovine APOBEC-3B:
(SEQ ID NO: 2485)
DGWEVAFRSGTVLKAGVLGVSMTEGWAGSGHPGQGACVWTPGTRNTMNLLREVLFKQQFGNQPRVPAP
YYRRKTYLCYQLKQRNDLTLDRGCFRNKKQRHAEIRFIDKINSLDLNPSQSYKIICYITWSPCPNCANELVN
FITRNNHLKLEIFASRLYFHWIKSFKMGLQDLQNAGISVAVMTHTEFEDCWEQFVDNQSRPFQPWDKLEQY
SASIRRRLQRILTAPI
Chimpanzee APOBEC-3B:
(SEQ ID NO: 2486)
MNPQIRNPMEWMYQRTFYYNFENEPILYGRSYTWLCYEVKIRRGHSNLLWDTGVFRGQMYSQPEHHAEM
CFLSWFCGNQLSAYKCFQITWFVSWTPCPDCVAKLAKFLAEHPNVTLTISAARLYYYWERDYRRALCRLS
QAGARVKIMDDEEFAYCWENFVYNEGQPFMPWYKFDDNYAFLHRTLKEHRHLMDPDTFTENENNDPLVL
RRHQTYLCYEVERLDNGTWVLMDQHMGFLCNEAKNLLCGFYGRHAELRELDLVPSLQLDPAQIYRVTWF
ISWSPCFSWGCAGQVRAFLQENTHVRLRIFAARIYDYDPLYKEALQMLRDAGAQVSIMTYDEFEYCWDTF
VYRQGCPFQPWDGLEEHSQALSGRLRAILQVRASSLCMVPHRPPPPPQSPGPCLPLCSEPPLGSLLPTGRPAP
SLPFLLTASFSFPPPASLPPLPSLSLSPGHLPVPSFHSLTSCSIQPPCSSRIRETEGWASVSKEGRDLG
Human APOBEC-3C:
(SEQ ID NO: 282)
MNPQIRNPMKAMYPGTFYFQFKNLWEANDRNETWLCFTVEGIKRRSVVSWKTGVFRNQVDSETHCHAER
CFLSWFCDDILSPNTKYQVTWYTSWSPCPDCAGEVAEFLARHSNVNLTIFTARLYYFQYPCYQEGLRSLSQEG
VAVEIMDYEDFKYCWENFVYNDNEPFKPWKGLKTNFRLLKRRLRESLQ (italic: nucleic
acid editing domain)
Gorilla APOBEC3C:
(SEQ ID NO: 2487)
MNPQIRNPMKAMYPGTFYFQFKNLWEANDRNETWLCFTVEGIKRRSVVSWKTGVFRNQVDSETHCHAER
CFLSWFCDDILSPNTNYQVTWYTSWSPCPECAGEVAEFLARHSNVNLTIFTARLYYFQDTDYQEGLRSLSQ
EGVAVKIMDYKDFKYCWENFVYNDDEPFKPWKGLKYNFRFLKRRLQEILE
Human APOBEC-3A:
(SEQ ID NO: 283)
MEASPASGPRHLMDPHIFTSNFNNGIGRHKTYLCYEVERLDNGTSVKMDQHRGFLHNQAKNLLCGFYGRH
AELRFLDLVPSLQLDPAQIYRVTWFISWSPCFSWGCAGEVRAFLQENTHVRLRIFAARIYDYDPLYKEALQML
RDAGAQVSIMTYDEFKHCWDTFVDHQGCPFQPWDGLDEHSQALSGRLRAILQNQGN
(italic: nucleic acid editing domain)
Rhesus macaque APOBEC-3A:
(SEQ ID NO: 2488)
MDGSPASRPRHLMDPNTFTFNENNDLSVRGRHQTYLCYEVERLDNGTWVPMDERRGFLCNKAKNVPCGD
YGCHVELRFLCEVPSWQLDPAQTYRVTWFISWSPCFRRGCAGQVRVFLQENKHVRLRIFAARIYDYDPLY
QEALRTLRDAGAQVSIMTYEEFKHCWDTFVDRQGRPFQPWDGLDEHSQALSGRLRAILQNQGN
Bovine APOBEC-3A:
(SEQ ID NO: 2489)
MDEYTFTENFNNQGWPSKTYLCYEMERLDGDATIPLDEYKGFVRNKGLDQPEKPCHAELYFLGKIHSWNL
DRNQHYRLTCFISWSPCYDCAQKLTTFLKENHHISLHILASRIYTHNRFGCHQSGLCELQAAGARITIMTFED
FKHCWETFVDHKGKPFQPWEGLNVKSQALCTELQAILKTQQN
Human APOBEC-3H:
(SEQ ID NO: 284)
MALLTAETFRLQFNNKRRLRRPYYPRKALLCYQLTPQNGSTPTRGYFENKKKCHAEICFINEIKSMGLDETQ
CYQVTCYLTWSPCSSCAWELVDFIKAHDHLNLGIFASRLYYHWCKPQQKGLRLLCGSQVPVEVMGFPKFAD
CWENFVDHEKPLSFNPYKMLEELDKNSRAIKRRLERIKIPGVRAQGRYMDILCDAEV
(italic: nucleic acid editing domain)
Rhesus macaque APOBEC-3H:
(SEQ ID NO: 2490)
MALLTAKTFSLQFNNKRRVNKPYYPRKALLCYQLTPQNGSTPTRGHLKNKKKDHAEIRFINKIKSMGLDET
QCYQVTCYLTWSPCPSCAGELVDFIKAHRHLNLRIFASRLYYHWRPNYQEGLLLLCGSQVPVEVMGLPEFT
DCWENFVDHKEPPSFNPSEKLEELDKNSQAIKRRLERIKSRSVDVLENGLRSLQLGPVTPSSSIRNSR
Human APOBEC-3D
(SEQ ID NO: 285)
MNPQIRNPMERMYRDTFYDNFENEPILYGRSYTWLCYEVKIKRGRSNLLWDTGVFRGPVLPKRQSNHRQE
VYFRFENHAEMCFLSWFCGNRLPANRRFQITWFVSWNPCLPCVVKVTKFLAEHPNVTLTISAARLYYYRDRD
WRWVLLRLHKAGARVKIMDYEDFAYCWENFVCNEGQPFMPWYKFDDNYASLHRTLKEILRNPMEAMYP
HIFYFHFKNLLKACGRNESWLCFTMEVTKHHSAVFRKRGVFRNQVDPETHCHAERCFLSWFCDDILSPNTN
YEVTWYTSWSPCPECAGEVAEFLARHSNVNLTIFTARLCYFWDTDYQEGLCSLSQEGASVKIMGYKDFVSC
WKNFVYSDDEPFKPWKGLQTNFRLLKRRLREILQ (italic: nucleic acid editing domain)
Human APOBEC-1
(SEQ ID NO: 286)
MTSEKGPSTGDPTLRRRIEPWEFDVFYDPRELRKEACLLYEIKWGMSRKIWRSSGKNTTNHVEVNFIKKFTS
ERDFHPSMSCSITWFLSWSPCWECSQAIREFLSRHPGVTLVIYVARLFWHMDQQNRQGLRDLVNSGVTIQI
MRASEYYHCWRNFVNYPPGDEAHWPQYPPLWMMLYALELHCIILSLPPCLKISRRWQNHLTFFRLHLQNC
HYQTIPPHILLATGLIHPSVAWR
Mouse APOBEC-1
(SEQ ID NO: 287)
MSSETGPVAVDPTLRRRIEPHEFEVFFDPRELRKETCLLYEINWGGRHSVWRHTSQNTSNHVEVNFLEKFTT
ERYFRPNTRCSITWFLSWSPCGECSRAITEFLSRHPYVTLFIYIARLYHHTDQRNRQGLRDLISSGVTIQIMTE
QEYCYCWRNFVNYPPSNEAYWPRYPHLWVKLYVLELYCHILGLPPCLKILRRKQPQLTFFTITLQTCHYQRI
PPHLLWATGLK
Rat APOBEC-1
(SEQ ID NO: 288)
MSSETGPVAVDPTLRRRIEPHEFEVFFDPRELRKETCLLYEINWGGRHSIWRHTSQNTNKHVEVNFIEKFTTE
RYFCPNTRCSITWFLSWSPCGECSRAITEFLSRYPHVTLFIYIARLYHHADPRNRQGLRDLISSGVTIQIMTEQ
ESGYCWRNFVNYSPSNEAHWPRYPHLWVRLYVLELYCULGLPPCLNILRRKQPQLTFFTIALQSCHYQRLP
PHILWATGLK
Human APOBEC-2:
(SEQ ID NO: 2491)
MAQKEEAAVATEAASQNGEDLENLDDPEKLKELIELPPFEIVTGERLPANFFKFQFRNVEYSSGRNKTFLCY
VVEAQGKGGQVQASRGYLEDEHAAAHAEEAFFNTILPAFDPALRYNVTWYVSSSPCAACADRIIKTLSKTK
NLRLLILVGRLFMWEEPEIQAALKKLKEAGCKLRIMKPQDFEYVWQNFVEQEEGESKAFQPWEDIQENFLY
YEEKLADILK
Mouse APOBEC-2:
(SEQ ID NO: 2492)
MAQKEEAAEAAAPASQNGDDLENLEDPEKLKELIDLPPFEIVTGVRLPVNFFKFQFRNVEYSSGRNKTFLC
YVVEVQSKGGQAQATQGYLEDEHAGAHAEEAFFNTILPAFDPALKYNVTWYVSSSPCAACADRILKTLSK
TKNLRLLILVSRLFMWEEPEVQAALKKLKEAGCKLRIMKPQDFEYIWQNFVEQEEGESKAFEPWEDIQENF
LYYEEKLADILK
Rat APOBEC-2:
(SEQ ID NO: 2493)
MAQKEEAAEAAAPASQNGDDLENLEDPEKLKELIDLPPFEIVTGVRLPVNFFKFQFRNVEYSSGRNKTFLC
YVVEAQSKGGQVQATQGYLEDEHAGAHABEAFFNTILPAFDPALKYNVTWYVSSSPCAACADRILKTLSK
TKNLRLLILVSRLFMWEEPEVQAALKKLKEAGCKLRIMKPQDFEYLWQNFVEQEEGESKAFEPWEDIQENF
LYYEEKLADILK
Bovine APOBEC-2:
(SEQ ID NO: 2494)
MAQKEEAAAAAEPASQNGEEVENLEDPEKLKELIELPPFEIVTGERLPAHYFKFQFRNVEYSSGRNKTFLCY
VVEAQSKGGQVQASRGYLEDEHATNHAREAFFNSIMPTFDPALRYMVTWYVSSSPCAACADRIVKTLNKT
KNLRLLILVGRLFMWEEPEIQAALRKLKEAGCRLRIMKPQDFEYIWQNFVEQEEGESKAFEPWEDIQENEL
YYEEKLADILK
Petromyzon marinus CDA1 (pmCDA1)
(SEQ ID NO: 289)
MTDAEYVRIHEKLDIYTFKKQFFNNKKSVSHRCYVLFELKRRGERRACFWGYAVNKPQSGTERGIHAEIFSI
RKVEEYLRDNPGQFTINWYSSWSPCADCAEKILEWYNQELRGNGHTLKIWACKLYYEKNARNQIGLWNL
RDNGVGLNVMVSEHYQCCRKIFIQSSHNQLNENRWLEKTLKRAEKRRSELSIMIQVKILHTTKSPAV
Human APOBEC3G D316R_D317R
(SEQ ID NO: 290)
MKPHFRNTVERMYRDTFSYNFYNRPILSRRNTVWLCYEVKTKGPSRPPLDAKIFRGQVYSELKYHPEMRFF
HWFSKWRKLHRDQEYEVTWYISWSPCTKCTRDMATFLAEDPKVTLTIFVARLYYFWDPDYQEALRSLCQ
KRDGPRATMKIMNYDEFQHCWSKFVYSQRELFEPWNNLPKYYILLHIMLGEILRHSMDPPTFTFNENNEPW
VRGRHETYLCYEVERMHNDTWVLLNQRRGFLCNQAPHKHGFLEGRHAELCFLDVIPFWKLDLDQDYRVT
CFTSWSPCFSCAQEMAKFISKNKHVSLCIFTARIYRRQGRCQEGLRTLAEAGAKISIMTYSEFKHCWDTFVD
HQGCPFQPWDGLDEHSQDLSGRLRAILQNQEN
Human APOBEC3G chain A
(SEQ ID NO: 291)
MDPPTFTFNFNNEPWVRGRHETYLCYEVERMHNDTWVLLNQRRGFLCNQAPHKHGFLEGRHAELCFLDV
IPFWKLDLDQDYRVTCFTSWSPCFSCAQEMAKFISKNKHVSLCIFTARIYDDQGRCQEGLRTLAEAGAKISI
MTYSEFKHCWDTFVDHQGCPFQPWDGLDEHSQDLSGRLRAILQ
Human APOBEC3G chain A D120R_D121R
(SEQ ID NO: 292)
MDPPTFTFNFNNEPWVRGRHETYLCYEVERMHNDTWVLLNQRRGFLCNQAPHKHGFLEGRHAELCFLDV
IPFWKLDLDQDYRVTCFTSWSPCFSCAQEMAKFISKNKHVSLCIFTARIYRRQGRCQEGLRTLAEAGAKISI
MTYSEFKHCWDTFVDHQGCPFQPWDGLDEHSQDLSGRLRAILQ

Non-limiting examples of fusion proteins/nucleobase editors are provided.

His₆-rAPOBEC1-XTEN-dCas9 for Escherichia coli expression
(SEQ ID NO: 293)
MGSSHHHHHHMSSETGPVAVDPTLRRRIEPHEFEVFFDPRELRKETCLLYEINWGGRHSIWRHTSQNTNKH
VEVNFIEKFTTERYFCPNTRCSITWFLSWSPCGECSRAITEFLSRYPHVTLFIYIARLYHHADPRNRQGLRDLI
SSGVTIQIMTEQESGYCWRNFVNYSPSNEAHWPRYPHLWVRLYVLELYCIILGLPPCLNILRRKQPQLTFFTI
ALQSCHYQRLPPHILWATGLKSGSETPGTSESATPESDKKYSIGLAIGTNSVGWAVITDEYKVPSKKFKVLG
NTDRHSIKKNLIGALLFDSGETAEATRLKRTARRRYTRRKNRICYLQEIFSNEMAKVDDSFFHRLEESFLVE
EDKKHERHPIFGNIVDEVAYHEKYPTIYHLRKKLVDSTDKADLRLIYLALAHMIKFRGHFLIEGDLNPDNSD
VDKLFIQLVQTYNQLFEENPINASGVDAKAILSARLSKSRRLENLIAQLPGEKKNGLEGNLIALSLGLTPNEK
SNFDLAEDAKLQLSKDTYDDDLDNLLAQIGDQYADLFLAAKNLSDAILLSDILRVNTEITKAPLSASMIKRY
DEHHQDLTLLKALVRQQLPEKYKEIFFDQSKNGYAGYIDGGASQEEFYKFIKPILEKMDGTEELLVKLNRE
DLLRKQRTFDNGSIPHQIHLGELHAILRRQEDFYPFLKDNREKIEKILTFRIPYYVGPLARGNSRFAWMTRKS
EETITPWNFEEVVDKGASAQSFIERMTNFDKNLPNEKVLPKHSLLYEYFTVYNELTKVKYVTEGMRKPAFL
SGEQKKAIVDLLFKTNRKVTVKQLKEDYFKKIECFDSVEISGVEDRFNASLGTYHDLLKIIKDKDFLDNEEN
EDILEDIVLTLTLFEDREMIEERLKTYAHLEDDKVMKQLKRRRYTGWGRLSRKLINGIRDKQSGKTILDFLK
SDGFANRNFMQLIHDDSLTFKEDIQKAQVSGQGDSLHEHIANLAGSPAIKKGILQTVKVVDELVKVMGRH
KPENIVIEMARENQTTQKGQKNSRERMKRIEEGIKELGSQILKEHPVENTQLQNEKLYLYYLQNGRDMYVD
QELDINRLSDYDVDAIVPQSFLKDDSIDNKVLTRSDKNRGKSDNVPSEEVVKKMKNYWRQLLNAKLITQR
KFDNLTKAERGGLSELDKAGFIKRQLVETRQITKHVAQILDSRMNTKYDENDKLIREVKVITLKSKLVSDFR
KDFQFYKVREINNYHHAHDAYLNAVVGTALIKKYPKLESEFVYGDYKVYDVRKMIAKSEQEIGKATAKYF
FYSNIMNFFKTEITLANGEIRKRPLIETNGETGEIVWDKGRDFATVRKVLSMPQVNIVKKTEVQTGGFSKESI
LPKRNSDKLIARKKDWDPKKYGGFDSPTVAYSVLVVAKVEKGKSKKLKSVKELLGITIMERSSFEKNPIDF
LEAKGYKEVKKDLIIKLPKYSLFELENGRKRMLASAGELQKGNELALPSKYVNFLYLASHYEKLKGSPEDN
EQKQLFVEQHKHYLDEIIEQISEFSKRVILADANLDKVLSAYNKHRDKPIREQAENIIHLFTLTNLGAPAAFK
YFDTTIDRKRYTSTKEVLDATLIHQSITGLYETRIDLSQLGGDSGGSPKKKRKV
rAPOBEC1-XTEN-dCas9-NLS for mammalian expression
(SEQ ID NO: 294)
MSSETGPVAVDPTLRRRIEPHEFEVFFDPRELRKETCLLYEINWGGRHSIWRHTSQNTNKHVEVNFIEKFTT
ERYFCPNTRCSITWFLSWSPCGECSRAITEFLSRYPHVTLFIYIARLYHHADPRNRQGLRDLISSGVTIQIMTE
QESGYCWRNFVNYSPSNEAHWPRYPHLWVRLYVLELYCIILGLPPCLNILRRKQPQLTFFTIALQSCHYQRL
PPHILWATGLKSGSETPGTSESATPESDKKYSIGLAIGTNSVGWAVITDEYKVPSKKFKVLGNTDRHSIKKN
LIGALLFDSGETAEATRLKRTARRRYTRRKNRICYLQEIFSNEMAKVDDSFFHRLEESELVEEDKKHERHPIF
GNIVDEVAYHEKYPTIYHLRKKLVDSTDKADLRLIYLALAHMIKFRGHFLIEGDLNPDNSDVDKLFIQLVQT
YNQLFEENPINASGVDAKAILSARLSKSRRLENLIAQLPGEKKNGLFGNLIALSLGLTPNFKSNFDLAEDAKL
QLSKDTYDDDLDNLLAQIGDQYADLFLAAKNLSDAILLSDILRVNTEITKAPLSASMIKRYDEHHQDLTLLK
ALVRQQLPEKYKEIFFDQSKNGYAGYIDGGASQEEFYKFIKPILEKMDGTEELLVKLNREDLLRKQRTFDN
GSIPHQIHLGELHAILRRQEDFYPFLKDNREKIEKILTFRIPYYVGPLARGNSRFAWMTRKSEETITPWNFEEV
VDKGASAQSFIERMTNEDKNLPNEKVLPKHSLLYEYFTVYNELTKVKYVTEGMRKPAFLSGEQKKAIVDL
LFKTNRKVTVKQLKEDYFKKIECFDSVEISGVEDRFNASLGTYHDLLKIIKDKDFLDNEENEDILEDIVLTLT
LFEDREMIEERLKTYAHLFDDKVMKQLKRRRYTGWGRLSRKLINGIRDKQSGKTILDFLKSDGFANRNFM
QLIHDDSLTFKEDIQKAQVSGQGDSLHEHIANLAGSPAIKKGILQTVKVVDELVKVMGRHKPENIVIEMARE
NQTTQKGQKNSRERMKRIEEGIKELGSQILKEHPVENTQLQNEKLYLYYLQNGRDMYVDQELDINRLSDY
DVDAIVPQSFLKDDSIDNKVLTRSDKNRGKSDNVPSEEVVKKMKNYWRQLLNAKLITQRKFDNLTKAERG
GLSELDKAGFIKRQLVETRQITKHVAQILDSRMNTKYDENDKLIREVKVITLKSKLVSDFRKDFQFYKVREI
NNYHHAHDAYLNAVVGTALIKKYPKLESEFVYGDYKVYDVRKMIAKSEQEIGKATAKYFFYSNIMNFFKT
EITLANGEIRKRPLIETNGETGEIVWDKGRDFATVRKVLSMPQVNIVKKTEVQTGGFSKESILPKRNSDKLIA
RKKDWDPKKYGGFDSPTVAYSVLVVAKVEKGKSKKLKSVKELLGITIMERSSFEKNPIDFLEAKGYKEVK
KDLIIKLPKYSLFELENGRKRMLASAGELQKGNELALPSKYVNFLYLASHYEKLKGSPEDNEQKQLFVEQH
KHYLDEHEQISEFSKRVILADANLDKVLSAYNKHRDKPIREQAENIIHLFTLTNLGAPAAFKYEDTTIDRKRY
TSTKEVLDATLIHQSITGLYETRIDLSQLGGDSGGSPKKKRKV
hAPOBEC1-XTEN-dCas9-NLS for mammalian expression
(SEQ ID NO: 295)
MTSEKGPSTGDPTLRRRIEPWEFDVFYDPRELRKEACLLYEIKWGMSRKIWRSSGKNTTNHVEVNFIKKFTS
ERDFHPSMSCSITWFLSWSPCWECSQAIREFLSRHPGVTLVIYVARLFWHMDQQNRQGLRDLVNSGVTIQI
MRASEYYHCWRNFVNYPPGDEAHWPQYPPLWMMLYALELHCIILSLPPCLKISRRWQNHLTFFRLHLQNC
HYQTIPPHILLATGLIHPSVAWRSGSETPGTSESATPESDKKYSIGLAIGTNSVGWAVITDEYKVPSKKFKVL
GNTDRHSIKKNLIGALLFDSGETAEATRLKRTARRRYTRRKNRICYLQEIFSNEMAKVDDSFFHRLEESFLV
EEDKKHERHPIFGNIVDEVAYHEKYPTIYHLRKKLVDSTDKADLRLIYLALAHMIKERGHFLIEGDLNPDNS
DVDKLFIQLVQTYNQLFEENPINASGVDAKAILSARLSKSRRLENLIAQLPGEKKNGLFGNLIALSLGLTPNF
KSNFDLAEDAKLQLSKDTYDDDLDNLLAQIGDQYADLFLAAKNLSDAILLSDILRVNTEITKAPLSASMIKR
YDEHHQDLTLLKALVRQQLPEKYKEIFFDQSKNGYAGYIDGGASQEEFYKFIKPILEKMDGTEELLVKLNR
EDLLRKQRTFDNGSIPHQIHLGELHAILRRQEDFYPFLKDNREKIEKILTFRIPYYVGPLARGNSRFAWMTRK
SEETITPWNFEEVVDKGASAQSFIERMTNFDKNLPNEKVLPKHSLLYEYFTVYNELTKVKYVTEGMRKPAF
LSGEQKKAIVDLLFKTNRKVTVKQLKEDYFKKIECFDSVEISGVEDRENASLGTYHDLLKIIKDKDFLDNEE
NEDILEDIVLTLTLFEDREMIEERLKTYAHLEDDKVMKQLKRRRYTGWGRLSRKLINGIRDKQSGKTILDFL
KSDGFANRNFMQLIHDDSLTFKEDIQKAQVSGQGDSLHEHIANLAGSPAIKKGILQTVKVVDELVKVMGR
HKPENIVIEMARENQTTQKGQKNSRERMKRIEEGIKELGSQILKEHPVENTQLQNEKLYLYYLQNGRDMYV
DQELDINRLSDYDVDAIVPQSFLKDDSIDNKVLTRSDKNRGKSDNVPSEEVVKKMKNYWRQLLNAKLITQ
RKFDNLTKAERGGLSELDKAGFIKRQLVETRQITKHVAQILDSRMNTKYDENDKLIREVKVITLKSKLVSDF
RKDFQFYKVREINNYHHAHDAYLNAVVGTALIKKYPKLESEFVYGDYKVYDVRKMIAKSEQEIGKATAK
YFFYSNIMNFFKTEITLANGEIRKRPLIETNGETGEIVWDKGRDFATVRKVLSMPQVNIVKKTEVQTGGFSK
ESILPKRNSDKLIARKKDWDPKKYGGFDSPTVAYSVLVVAKVEKGKSKKLKSVKELLGITIMERSSFEKNPI
DFLEAKGYKEVKKDLIIKLPKYSLFELENGRKRMLASAGELQKGNELALPSKYVNFLYLASHYEKLKGSPE
DNEQKQLFVEQHKHYLDEIIEQISEFSKRVILADANLDKVLSAYNKHRDKPIREQAENIIHLFTLTNLGAPAA
FKYFDTTIDRKRYTSTKEVLDATLIHQSITGLYETRIDLSQLGGDSGGSPKKKRKV
rAPOBEC1-XTEN-dCas9-UGI-NLS
(SEQ ID NO: 296)
MSSETGPVAVDPTLRRRIEPHEFEVFFDPRELRKETCLLYEINWGGRHSIWRHTSQNTNKHVEVNFIEKFTT
ERYFCPNTRCSITWFLSWSPCGECSRAITEFLSRYPHVTLFIYIARLYHHADPRNRQGLRDLISSGVTIQIMTE
QESGYCWRNFVNYSPSNEAHWPRYPHLWVRLYVLELYCIILGLPPCLNILRRKQPQLTFFTIALQSCHYQRL
PPHILWATGLKSGSETPGTSESATPESDKKYSIGLAIGTNSVGWAVITDEYKVPSKKFKVLGNTDRHSIKKN
LIGALLFDSGETAEATRLKRTARRRYTRRKNRICYLQEIFSNEMAKVDDSFFHRLEESFLVEEDKKHERHPIF
GNIVDEVAYHEKYPTIYHLRKKLVDSTDKADLRLIYLALAHMIKFRGHFLIEGDLNPDNSDVDKLFIQLVQT
YNQLFEENPINASGVDAKAILSARLSKSRRLENLIAQLPGEKKNGLFGNLIALSLGLTPNFKSNFDLAEDAKL
QLSKDTYDDDLDNLLAQIGDQYADLFLAAKNLSDAILLSDILRVNTEITKAPLSASMIKRYDEHHQDLTLLK
ALVRQQLPEKYKEIFFDQSKNGYAGYIDGGASQEEFYKFIKPILEKMDGTEELLVKLNREDLLRKQRTFDN
GSIPHQIHLGELHAILRRQEDFYPFLKDNREKIEKILTFRIPYYVGPLARGNSRFAWMTRKSEETITPWNFEEV
VDKGASAQSFIERMTNEDKNLPNEKVLPKHSLLYEYFTVYNELTKVKYVTEGMRKPAFLSGEQKKAIVDL
LFKTNRKVTVKQLKEDYFKKIECFDSVEISGVEDRENASLGTYHDLLKIIKDKDFLDNEENEDILEDIVLTLT
LFEDREMIEERLKTYAHLFDDKVMKQLKRRRYTGWGRLSRKLINGIRDKQSGKTILDFLKSDGFANRNEM
QLIHDDSLTFKEDIQKAQVSGQGDSLHEHIANLAGSPAIKKGILQTVKVVDELVKVMGRHKPENIVIEMARE
NQTTQKGQKNSRERMKRIEEGIKELGSQILKEHPVENTQLQNEKLYLYYLQNGRDMYVDQELDINRLSDY
DVDAIVPQSFLKDDSIDNKVLTRSDKNRGKSDNVPSEEVVKKMKNYWRQLLNAKLITQRKFDNLTKAERG
GLSELDKAGFIKRQLVETRQITKHVAQILDSRMNTKYDENDKLIREVKVITLKSKLVSDFRKDFQFYKVREI
NNYHHAHDAYLNAVVGTALIKKYPKLESEFVYGDYKVYDVRKMIAKSEQEIGKATAKYFFYSNIMNFFKT
EITLANGEIRKRPLIETNGETGEIVWDKGRDFATVRKVLSMPQVNIVKKTEVQTGGFSKESILPKRNSDKLIA
RKKDWDPKKYGGFDSPTVAYSVLVVAKVEKGKSKKLKSVKELLGITIMERSSFEKNPIDFLEAKGYKEVK
KDLIIKLPKYSLFELENGRKRMLASAGELQKGNELALPSKYVNFLYLASHYEKLKGSPEDNEQKQLFVEQH
KHYLDEIIEQISEFSKRVILADANLDKVLSAYNKHRDKPIREQAENIIHLFTLTNLGAPAAFKYFDTTIDRKRY
TSTKEVLDATLIHQSITGLYETRIDLSQLGGDSGGSTNLSDIIEKETGKQLVIQESILMLPEEVEEVIGNKPESD
ILVHTAYDESTDENVMLLTSDAPEYKPWALVIQDSNGENKIKMLSGGSPKKKRKV
rAPOBEC1-XTEN-Cas9 nickase-UGI-NLS
(BE3, SEQ ID NO: 297)
MSSETGPVAVDPTLRRRIEPHEFEVFFDPRELRKETCLLYEINWGGRHSIWRHTSQNTNKHVEVNFIEKFTT
ERYFCPNTRCSITWFLSWSPCGECSRAITEFLSRYPHVTLFIYIARLYHHADPRNRQGLRDLISSGVTIQIMTE
QESGYCWRNFVNYSPSNEAHWPRYPHLWVRLYVLELYCIILGLPPCLNILRRKQPQLTFFTIALQSCHYQRL
PPHILWATGLKSGSETPGTSESATPESDKKYSIGLAIGTNSVGWAVITDEYKVPSKKFKVLGNTDRHSIKKN
LIGALLFDSGETAEATRLKRTARRRYTRRKNRICYLQEIFSNEMAKVDDSFFHRLEESFLVEEDKKHERHPIF
GNIVDEVAYHEKYPTIYHLRKKLVDSTDKADLRLIYLALAHMIKFRGHFLIEGDLNPDNSDVDKLFIQLVQT
YNQLFEENPINASGVDAKAILSARLSKSRRLENLIAQLPGEKKNGLFGNLIALSLGLTPNFKSNEDLAEDAKL
QLSKDTYDDDLDNLLAQIGDQYADLFLAAKNLSDAILLSDILRVNTEITKAPLSASMIKRYDEHHQDLTLLK
ALVRQQLPEKYKEIFFDQSKNGYAGYIDGGASQEEFYKFIKPILEKMDGTEELLVKLNREDLLRKQRTEDN
GSIPHQIHLGELHAILRRQEDFYPFLKDNREKIEKILTFRIPYYVGPLARGNSRFAWMTRKSEETITPWNFEEV
VDKGASAQSFIERMTNFDKNLPNEKVLPKHSLLYEYFTVYNELTKVKYVTEGMRKPAFLSGEQKKAIVDL
LFKTNRKVTVKQLKEDYFKKIECFDSVEISGVEDRFNASLGTYHDLLKIIKDKDELDNEENEDILEDIVLTITL
FEDREMIEERLKTYAHLFDDKVMKQLKRRRYTGWGRLSRKLINGIRDKQSGKTILDFLKSDGFANRNFMQ
LIHDDSLTFKEDIQKAQVSGQGDSLHEHIANLAGSPAIKKGILQTVKVVDELVKVMGRHKPENIVIEMAREN
QTTQKGQKNSRERMKRIEEGIKELGSQILKEHPVENTQLQNEKLYLYYLQNGRDMYVDQELDINRLSDYD
VDHIVPQSFLKDDSIDNKVLTRSDKNRGKSDNVPSEEVVKKMKNYWRQLLNAKLITQRKEDNLTKAERGG
LSELDKAGFIKRQLVETRQITKHVAQILDSRMNTKYDENDKLIREVKVITLKSKLVSDFRKDFQFYKVREIN
NYHHAHDAYLNAVVGTALIKKYPKLESEFVYGDYKVYDVRKMIAKSEQEIGKATAKYFFYSNIMNFFKTE
ITLANGEIRKRPLIETNGETGEIVWDKGRDFATVRKVLSMPQVNIVKKTEVQTGGFSKESILPKRNSDKLIAR
KKDWDPKKYGGFDSPTVAYSVLVVAKVEKGKSKKLKSVKELLGITIMERSSFEKNPIDFLEAKGYKEVKK
DLIIKLPKYSLFELENGRKRMLASAGELQKGNELALPSKYVNFLYLASHYEKLKGSPEDNEQKQLFVEQHK
HYLDEIIEQISEFSKRVILADANLDKVLSAYNKHRDKPIREQAENIIHLFTLTNLGAPAAFKYFDTTIDRKRYT
STKEVLDATLIHQSITGLYETRIDLSQLGGDSGGSTNLSDIIEKETGKQLVIQESILMLPEEVEEVIGNKPESDI
LVHTAYDESTDENVMLLTSDAPEYKPWALVIQDSNGENKIKMLSGGSPKKKRKV
pmCDA1-XTEN-dCas9-UGI (bacteria)
(SEQ ID NO: 298)
MTDAEYVRIHEKLDIYTFKKQFFNNKKSVSHRCYVLFELKRRGERRACFWGYAVNKPQSGTERGIHAEIFSI
RKVEEYLRDNPGQFTINWYSSWSPCADCAEKILEWYNQELRGNGHTLKIWACKLYYEKNARNQIGLWNL
RDNGVGLNVMVSEHYQCCRKIFIQSSHNQLNENRWLEKTLKRAEKRRSELSIMIQVKILHTTKSPAVSGSET
PGTSESATPESDKKYSIGLAIGTNSVGWAVITDEYKVPSKKFKVLGNTDRHSIKKNLIGALLFDSGETAEAT
RLKRTARRRYTRRKNRICYLQEIFSNEMAKVDDSFFHRLEESFLVEEDKKHERHPIFGNIVDEVAYHEKYPT
IYHLRKKLVDSTDKADLRLIYLALAHMIKERGHFLIEGDLNPDNSDVDKLFIQLVQTYNQLFEENPINASGV
DAKAILSARLSKSRRLENLIAQLPGEKKNGLFGNLIALSLGLTPNFKSNFDLAEDAKLQLSKDTYDDDLDNL
LAQIGDQYADLFLAAKNLSDAILLSDILRVNTEITKAPLSASMIKRYDEHHQDLTLLKALVRQQLPEKYKEI
FFDQSKNGYAGYIDGGASQEEFYKFIKPILEKMDGTEELLVKLNREDLLRKQRTFDNGSIPHQIHLGELHAIL
RRQEDFYPFLKDNREKIEKILTFRIPYYVGPLARGNSRFAWMTRKSEETITPWNFEEVVDKGASAQSFIERM
TNFDKNLPNEKVLPKHSLLYEYFTVYNELTKVKYVTEGMRKPAFLSGEQKKAIVDLLFKTNRKVTVKQLK
EDYFKKIECFDSVEISGVEDRFNASLGTYHDLLKIIKDKDFLDNEENEDILEDIVLTLTLFEDREMIEERLKTY
AHLFDDKVMKQLKRRRYTGWGRLSRKLINGIRDKQSGKTILDFLKSDGFANRNFMQLIHDDSLTFKEDIQK
AQVSGQGDSLHEHIANLAGSPAIKKGILQTVKVVDELVKVMGRHKPENIVIEMARENQTTQKGQKNSRER
MKRIEEGIKELGSQILKEHPVENTQLQNEKLYLYYLQNGRDMYVDQELDINRLSDYDVDAIVPQSFLKDDSI
DNKVLTRSDKNRGKSDNVPSEEVVKKMKNYWRQLLNAKLITQRKFDNLTKAERGGLSELDKAGFIKRQL
VETRQITKHVAQILDSRMNTKYDENDKLIREVKVITLKSKLVSDERKDFQFYKVREINNYHHAHDAYLNAV
VGTALIKKYPKLESEFVYGDYKVYDVRKMIAKSEQEIGKATAKYFFYSNIMNFFKTEITLANGEIRKRPLIET
NGETGEIVWDKGRDFATVRKVLSMPQVNIVKKTEVQTGGFSKESILPKRNSDKLIARKKDWDPKKYGGFD
SPTVAYSVLVVAKVEKGKSKKLKSVKELLGITIMERSSFEKNPIDFLEAKGYKEVKKDLIIKLPKYSLFELEN
GRKRMLASAGELQKGNELALPSKYVNFLYLASHYEKLKGSPEDNEQKQLFVEQHKHYLDEIIEQISEFSKR
VILADANLDKVLSAYNKHRDKPIREQAENIIHLFTLTNLGAPAAFKYFDTTIDRKRYTSTKEVLDATLIHQSI
TGLYETRIDLSQLGGDSGGSMTNLSDIIEKETGKQLVIQESILMLPEEVEEVIGNKPESDILVHTAYDESTDEN
VMLLTSDAPEYKPWALVIQDSNGENKIKML
pmCDA1-XTEN-nCas9-UGI-NLS (mammalian construct)
(SEQ ID NO: 299)
MTDAEYVRIHEKLDIYTFKKQFFNNKKSVSHRCYVLFELKRRGERRACFWGYAVNKPQSGTERGIHAEIFSI
RKVEEYLRDNPGQFTINWYSSWSPCADCAEKILEWYNQELRGNGHTLKIWACKLYYEKNARNQIGLWNL
RDNGVGLNVMVSEHYQCCRKIFIQSSHNQLNENRWLEKTLKRAEKRRSELSIMIQVKILHTTKSPAVSGSET
PGTSESATPESDKKYSIGLAIGTNSVGWAVITDEYKVPSKKFKVLGNTDRHSIKKNLIGALLFDSGETAEAT
RLKRTARRRYTRRKNRICYLQEIFSNEMAKVDDSFFHRLEESFLVEEDKKHERHPIFGNIVDEVAYHEKYPT
IYHLRKKLVDSTDKADLRLIYLALAHMIKERGHFLIEGDLNPDNSDVDKLFIQLVQTYNQLFEENPINASGV
DAKAILSARLSKSRRLENLIAQLPGEKKNGLFGNLIALSLGLTPNFKSNFDLAEDAKLQLSKDTYDDDLDNL
LAQIGDQYADLFLAAKNLSDAILLSDILRVNTEITKAPLSASMIKRYDEHHQDLTLLKALVRQQLPEKYKEI
FFDQSKNGYAGYIDGGASQEEFYKFIKPILEKMDGTEELLVKLNREDLLRKQRTFDNGSIPHQIHLGELHAIL
RRQEDFYPFLKDNREKIEKILTFRIPYYVGPLARGNSRFAWMTRKSEETITPWNFEEVVDKGASAQSFIERM
TNFDKNLPNEKVLPKHSLLYEYFTVYNELTKVKYVTEGMRKPAFLSGEQKKAIVDLLFKTNRKVTVKQLK
EDYFKKIECFDSVEISGVEDRFNASLGTYHDLLKIIKDKDFLDNEENEDILEDIVLTLTLFEDREMIEERLKTY
AHLFDDKVMKQLKRRRYTGWGRLSRKLINGIRDKQSGKTILDFLKSDGFANRNFMQLIHDDSLTFKEDIQK
AQVSGQGDSLHEHIANLAGSPAIKKGILQTVKVVDELVKVMGRHKPENIVIEMARENQTTQKGQKNSRER
MKRIEEGIKELGSQILKEHPVENTQLQNEKLYLYYLQNGRDMYVDQELDINRLSDYDVDHIVPQSFLKDDSI
DNKVLTRSDKNRGKSDNVPSEEVVKKMKNYWRQLLNAKLITQRKFDNLTKAERGGLSELDKAGFIKRQL
VETRQITKHVAQILDSRMNTKYDENDKLIREVKVITLKSKLVSDFRKDFQFYKVREINNYHHAHDAYLNAV
VGTALIKKYPKLESEFVYGDYKVYDVRKMIAKSEQEIGKATAKYFFYSNIMNFFKTEITLANGEIRKRPLIET
NGETGEIVWDKGRDFATVRKVLSMPQVNIVKKTEVQTGGFSKESILPKRNSDKLIARKKDWDPKKYGGFD
SPTVAYSVLVVAKVEKGKSKKLKSVKELLGITIMERSSFEKNPIDFLEAKGYKEVKKDLIIKLPKYSLFELEN
GRKRMLASAGELQKGNELALPSKYVNFLYLASHYEKLKGSPEDNEQKQLFVEQHKHYLDEIIEQISEFSKR
VILADANLDKVLSAYNKHRDKPIREQAENIIHLFTLTNLGAPAAFKYFDTTIDRKRYTSTKEVLDATLIHQSI
TGLYETRIDLSQLGGDSGGSTNLSDIIEKETGKQLVIQESILMLPEEVEEVIGNKPESDILVHTAYDESTDENV
MLLTSDAPEYKPWALVIQDSNGENKIKMLSGGSPKKKRKV
huAPOBEC3G-XTEN-dCas9-UGI (bacteria)
(SEQ ID NO: 300)
MDPPTFTFNFNNEPWVRGRHETYLCYEVERMHNDTWVLLNQRRGFLCNQAPHKHGFLEGRHAELCFLDV
IPFWKLDLDQDYRVTCFTSWSPCFSCAQEMAKFISKNKHVSLCIFTARIYDDQGRCQEGLRTLAEAGAKISI
MTYSEFKHCWDTFVDHQGCPFQPWDGLDEHSQDLSGRLRAILQSGSETPGTSESATPESDKKYSIGLAIGTN
SVGWAVITDEYKVPSKKFKVLGNTDRHSIKKNLIGALLFDSGETAEATRLKRTARRRYTRRKNRICYLQEIF
SNEMAKVDDSFFHRLEESFLVEEDKKHERHPIFGNIVDEVAYHEKYPTIYHLRKKLVDSTDKADLRLIYLAL
AHMIKFRGHFLIEGDLNPDNSDVDKLFIQLVQTYNQLFEENPINASGVDAKAILSARLSKSRRLENLIAQLPG
EKKNGLFGNLIALSLGLTPNFKSNEDLAEDAKLQLSKDTYDDDLDNLLAQIGDQYADLFLAAKNLSDAILL
SDILRVNTEITKAPLSASMIKRYDEHHQDLTLLKALVRQQLPEKYKEIFFDQSKNGYAGYIDGGASQEEFYK
FIKPILEKMDGTEELLVKLNREDLLRKQRTFDNGSIPHQIHLGELHAILRRQEDFYPFLKDNREKIEKILTFRIP
YYVGPLARGNSRFAWMTRKSEETITPWNFEEVVDKGASAQSFIERMTNFDKNLPNEKVLPKHSLLYEYFT
VYNELTKVKYVTEGMRKPAFLSGEQKKAIVDLLFKTNRKVTVKQLKEDYFKKIECFDSVEISGVEDRENAS
LGTYHDLLKIIKDKDFLDNEENEDILEDIVLTLTLFEDREMIEERLKTYAHLFDDKVMKQLKRRRYTGWGR
LSRKLINGIRDKQSGKTILDFLKSDGFANRNFMQLIHDDSLTFKEDIQKAQVSGQGDSLHEHIANLAGSPAIK
KGILQTVKVVDELVKVMGRHKPENIVIEMARENQTTQKGQKNSRERMKRIEEGIKELGSQILKEHPVENTQ
LQNEKLYLYYLQNGRDMYVDQELDINRLSDYDVDAIVPQSFLKDDSIDNKVLTRSDKNRGKSDNVPSEEV
VKKMKNYWRQLLNAKLITQRKFDNLTKAERGGLSELDKAGFIKRQLVETRQITKHVAQILDSRMNTKYDE
NDKLIREVKVITLKSKLVSDFRKDFQFYKVREINNYHHAHDAYLNAVVGTALIKKYPKLESEFVYGDYKV
YDVRKMIAKSEQEIGKATAKYFFYSNIMNFFKTEITLANGEIRKRPLIEINGETGEIVWDKGRDFATVRKVL
SMPQVNIVKKTEVQTGGFSKESILPKRNSDKLIARKKDWDPKKYGGFDSPTVAYSVLVVAKVEKGKSKKL
KSVKELLGITIMERSSFEKNPIDFLEAKGYKEVKKDLIIKLPKYSLFELENGRKRMLASAGELQKGNELALPS
KYVNFLYLASHYEKLKGSPEDNEQKQLFVEQHKHYLDEIIEQISEFSKRVILADANLDKVLSAYNKHRDKPI
REQAENIIHLFTLTNLGAPAAFKYFDTTIDRKRYTSTKEVLDATLIHQSITGLYETRIDLSQLGGDSGGSMTN
LSDIIEKETGKQLVIQESILMLPEEVEEVIGNKPESDILVHTAYDESTDENVMLLTSDAPEYKPWALVIQDSN
GENKIKML
huAPOBEC3G-XTEN-nCas9-UGI-NLS (mammalian construct)
(SEQ ID NO: 301)
MDPPTFTFNENNEPWVRGRHETYLCYEVERMHNDTWVLLNQRRGFLCNQAPHKHGFLEGRHAELCFLDV
IPFWKLDLDQDYRVTCFTSWSPCFSCAQEMAKFISKNKHVSLCIFTARIYDDQGRCQEGLRTLAEAGAKISI
MTYSEFKHCWDTFVDHQGCPFQPWDGLDEHSQDLSGRLRAILQSGSETPGTSESATPESDKKYSIGLAIGTN
SVGWAVITDEYKVPSKKFKVLGNTDRHSIKKNLIGALLEDSGETAEATRLKRTARRRYTRRKNRICYLQEIF
SNEMAKVDDSFFHRLEESFLVEEDKKHERHPIFGNIVDEVAYHEKYPTIYHLRKKLVDSTDKADLRLIYLAL
AHMIKFRGHFLIEGDLNPDNSDVDKLFIQLVQTYNQLFEENPINASGVDAKAILSARLSKSRRLENLIAQLPG
EKKNGLFGNLIALSLGLTPNFKSNFDLAEDAKLQLSKDTYDDDLDNLLAQIGDQYADLFLAAKNLSDAILL
SDILRVNTEITKAPLSASMIKRYDEHHQDLTLLKALVRQQLPEKYKEIFFDQSKNGYAGYIDGGASQEEFYK
FIKPILEKMDGTEELLVKLNREDLLRKQRTFDNGSIPHQIHLGELHAILRRQEDFYPFLKDNREKIEKILTFRIP
YYVGPLARGNSRFAWMTRKSEETITPWNFEEVVDKGASAQSFIERMTNFDKNLPNEKVLPKHSLLYEYFT
VYNELTKVKYVTEGMRKPAFLSGEQKKAIVDLLFKTNRKVTVKQLKEDYFKKIECFDSVEISGVEDRENAS
LGTYHDLLKIIKDKDFLDNEENEDILEDIVLTLTLFEDREMIEERLKTYAHLFDDKVMKQLKRRRYTGWGR
LSRKLINGIRDKQSGKTILDFLKSDGFANRNFMQLIHDDSLTFKEDIQKAQVSGQGDSLHEHIANLAGSPAIK
KGILQTVKVVDELVKVMGRHKPENIVIEMARENQTTQKGQKNSRERMKRIEEGIKELGSQILKEHPVENTQ
LQNEKLYLYYLQNGRDMYVDQELDINRLSDYDVDHIVPQSFLKDDSIDNKVLTRSDKNRGKSDNVPSEEV
VKKMKNYWRQLLNAKLITQRKFDNLTKAERGGLSELDKAGFIKRQLVETRQITKHVAQILDSRMNTKYDE
NDKLIREVKVITLKSKLVSDFRKDFQFYKVREINNYHHAHDAYLNAVVGTALIKKYPKLESEFVYGDYKV
YDVRKMIAKSEQEIGKATAKYFFYSNIMNFFKTEITLANGEIRKRPLIETNGETGEIVWDKGRDFATVRKVL
SMPQVNIVKKTEVQTGGFSKESILPKRNSDKLIARKKDWDPKKYGGFDSPTVAYSVLVVAKVEKGKSKKL
KSVKELLGITIMERSSFEKNPIDFLEAKGYKEVKKDLIIKLPKYSLFELENGRKRMLASAGELQKGNELALPS
KYVNFLYLASHYEKLKGSPEDNEQKQLFVEQHKHYLDEIIEQISEFSKRVILADANLDKVLSAYNKHRDKPI
REQAENIIHLFTLTNLGAPAAFKYFDTTIDRKRYTSTKEVLDATLIHQSITGLYETRIDLSQLGGDSGGSTNLS
DIIEKETGKQLVIQESILMLPEEVEEVIGNKPESDILVHTAYDESTDENVMLLTSDAPEYKPWALVIQDSNGE
NKIKMLSGGSPKKKRKV
huAPOBEC3G (D316R_D317R)-XTEN-nCas9-UGI-NLS (mammalian construct)
(SEQ ID NO: 302)
MDPPTFTFNFNNEPWVRGRHETYLCYEVERMHNDTWVLLNQRRGFLCNQAPHKHGFLEGRHAELCFLDV
IPFWKLDLDQDYRVTCFTSWSPCFSCAQEMAKFISKNKHVSLCIFTARIYRRQGRCQEGLRTLAEAGAKISI
MTYSEFKHCWDTFVDHQGCPFQPWDGLDEHSQDLSGRLRAILQSGSETPGTSESATPESDKKYSIGLAIGTN
SVGWAVITDEYKVPSKKFKVLGNTDRHSIKKNLIGALLEDSGETAEATRLKRTARRRYTRRKNRICYLQEIF
SNEMAKVDDSFFHRLEESFLVEEDKKHERHPIFGNIVDEVAYHEKYPTIYHLRKKLVDSTDKADLRLIYLAL
AHMIKFRGHFLIEGDLNPDNSDVDKLFIQLVQTYNQLFEENPINASGVDAKAILSARLSKSRRLENLIAQLPG
EKKNGLFGNLIALSLGLTPNFKSNFDLAEDAKLQLSKDTYDDDLDNLLAQIGDQYADLFLAAKNLSDAILL
SDILRVNTEITKAPLSASMIKRYDEHHQDLTLLKALVRQQLPEKYKEIFFDQSKNGYAGYIDGGASQEEFYK
FIKPILEKMDGTEELLVKLNREDLLRKQRTFDNGSIPHQIHLGELHAILRRQEDFYPFLKDNREKIEKILTFRIP
YYVGPLARGNSRFAWMTRKSEETITPWNFEEVVDKGASAQSFIERMTNFDKNLPNEKVLPKHSLLYEYFT
VYNELTKVKYVTEGMRKPAFLSGEQKKAIVDLLFKTNRKVTVKQLKEDYFKKIECFDSVEISGVEDRENAS
LGTYHDLLKIIKDKDFLDNEENEDILEDIVLTLTLFEDREMIEERLKTYAHLEDDKVMKQLKRRRYTGWGR
LSRKLINGIRDKQSGKTILDFLKSDGFANRNFMQLIHDDSLTFKEDIQKAQVSGQGDSLHEHIANLAGSPAIK
KGILQTVKVVDELVKVMGRHKPENIVIEMARENQTTQKGQKNSRERMKRIEEGIKELGSQILKEHPVENTQ
LQNEKLYLYYLQNGRDMYVDQELDINRLSDYDVDHIVPQSFLKDDSIDNKVLTRSDKNRGKSDNVPSEEV
VKKMKNYWRQLLNAKLITQRKFDNLTKAERGGLSELDKAGFIKRQLVETRQITKHVAQILDSRMNTKYDE
NDKLIREVKVITLKSKLVSDFRKDFQFYKVREINNYHHAHDAYLNAVVGTALIKKYPKLESEFVYGDYKV
YDVRKMIAKSEQEIGKATAKYFFYSNIMNFFKTEITLANGEIRKRPLIETNGETGEIVWDKGRDFATVRKVL
SMPQVNIVKKTEVQTGGFSKESILPKRNSDKLIARKKDWDPKKYGGFDSPTVAYSVLVVAKVEKGKSKKL
KSVKELLGITIMERSSFEKNPIDFLEAKGYKEVKKDLIIKLPKYSLFELENGRKRMLASAGELQKGNELALPS
KYVNFLYLASHYEKLKGSPEDNEQKQLFVEQHKHYLDEIIEQISEFSKRVILADANLDKVLSAYNKHRDKPI
REQAENIIHLFTLTNLGAPAAFKYFDTTIDRKRYTSTKEVLDATLIHQSITGLYETRIDLSQLGGDSGGSTNLS
DIIEKETGKQLVIQESILMLPEEVEEVIGNKPESDILVHTAYDESTDENVMLLTSDAPEYKPWALVIQDSNGE
NKIKMLSGGSPKKKRKV
Base Editor 4 (BE4; APOBEC1-linker(32 aa)-Cas9n(D10A)-linker(9 aa)-
UGI-linker(9 aa)-UGI)
(SEQ ID NO: 2495)
MSSETGPVAVDPTLRRRIEPHEFEVFFDPRELRKETCLLYEINWGGRHSIWRHTSQNTNKHVEVNFIEKFTT
ERYFCPNTRCSITWFLSWSPCGECSRAITEFLSRYPHVTLFIYIARLYHHADPRNRQGLRDLISSGVTIQIMTE
QESGYCWRNFVNYSPSNEAHWPRYPHLWVRLYVLELYCIILGLPPCLNILRRKQPQLTFFTIALQSCHYQRL
PPHILWATGLKSGGSSGGSSGSETPGTSESATPESSGGSSGGSDKKYSIGLAIGTNSVGWAVITDEYKVPSKK
FKVLGNTDRHSIKKNLIGALLFDSGETAEATRLKRTARRRYTRRKNRICYLQEIFSNEMAKVDDSFFHRLEE
SFLVEEDKKHERHPIFGNIVDEVAYHEKYPTIYHLRKKLVDSTDKADLRLIYLALAHMIKFRGHFLIEGDLN
PDNSDVDKLFIQLVQTYNQLFEENPINASGVDAKAILSARLSKSRRLENLIAQLPGEKKNGLFGNLIALSLGL
TPNFKSNFDLAEDAKLQLSKDTYDDDLDNLLAQIGDQYADLFLAAKNLSDAILLSDILRVNTEITKAPLSAS
MIKRYDEHHQDLTLLKALVRQQLPEKYKEIFFDQSKNGYAGYIDGGASQEEFYKFIKPILEKMDGTEELLV
KLNREDLLRKQRTFDNGSIPHQIHLGELHAILRRQEDFYPFLKDNREKIEKILTFRIPYYVGPLARGNSRFAW
MTRKSEETITPWNFEEVVDKGASAQSFIERMTNFDKNLPNEKVLPKHSLLYEYFTVYNELTKVKYVTEGM
RKPAFLSGEQKKAIVDLLFKTNRKVTVKQLKEDYFKKIECFDSVEISGVEDRFNASLGTYHDLLKIIKDKDF
LDNEENEDILEDIVLTLTLFEDREMIEERLKTYAHLFDDKVMKQLKRRRYTGWGRLSRKLINGIRDKQSGK
TILDFLKSDGFANRNFMQLIHDDSLTFKEDIQKAQVSGQGDSLHEHIANLAGSPAIKKGILQTVKVVDELVK
VMGRHKPENIVIEMARENQTTQKGQKNSRERMKRIEEGIKELGSQILKEHPVENTQLQNEKLYLYYLQNGR
DMYVDQELDINRLSDYDVDHIVPQSFLKDDSIDNKVLTRSDKNRGKSDNVPSEEVVKKMKNYWRQLLNA
KLITQRKFDNLTKAERGGLSELDKAGFIKRQLVETRQITKHVAQILDSRMNTKYDENDKLIREVKVITLKSK
LVSDFRKDFQFYKVREINNYHHAHDAYLNAVVGTALIKKYPKLESEFVYGDYKVYDVRKMIAKSEQEIGK
ATAKYFFYSNIMNFFKTEITLANGEIRKRPLIETNGETGEIVWDKGRDFATVRKVLSMPQVNIVKKTEVQTG
GFSKESILPKRNSDKLIARKKDWDPKKYGGFDSPTVAYSVLVVAKVEKGKSKKLKSVKELLGITIMERSSFE
KNPIDFLEAKGYKEVKKDLIIKLPKYSLFELENGRKRMLASAGELQKGNELALPSKYVNFLYLASHYEKLK
GSPEDNEQKQLFVEQHKHYLDEIIEQISEFSKRVILADANLDKVLSAYNKHRDKPIREQAENIIHLFTLTNLG
APAAFKYFDTTIDRKRYTSTKEVLDATLIHQSITGLYETRIDLSQLGGDSGGSGGSGGSTNLSDIIEKETGKQ
LVIQESILMLPEEVEEVIGNKPESDILVHTAYDESTDENVMLLTSDAPEYKPWALVIQDSNGENKIKMLSGG
SGGSGGSTNLSDIIEKETGKQLVIQESILMLPEEVEEVIGNKPESDILVHTAYDESTDENVMLLTSDAPEYKP
WALVIQDSNGENKIKMLSGGSPKKKRK

### Example 2: CRISPR/Cas9 Genome/Base-Editing Methods for Modifying Ion Channels in Dorsal Root Ganglia (DRG)

Long-term chronic pain due to trauma and associated with advanced cancer remains an unmet medical need. Management of pain using painkillers is inherently limited by the development of tolerance, physiological dependence, progressive addiction, and potential for overdose. Current health policies in response to the massive demand for painkillers (˜80-100 million patients/year in the US) have led to extensive prescription of opioids, inadvertently contributing to broader public challenges associated with substance abuse and drug-related crime. Fundamentally, there is a pressing need for an innovative solution to address chronic pain that is non-addictive, generalizable, and permanent.

A normal physiological outcome of trauma, inflammation, and nerve injury is the induction of gene expression changes in neighboring nociceptive neurons during the period required for healing, for example by facilitating the firing of action potentials by neurons at a lower activation threshold. These gene expression changes underlie the sensations of hyperalgesia (increased pain sensitivity) and allodynia (pain following a normally innocuous stimulus). Chronic pain develops when the enhanced sensitization of sensory neurons becomes irreversibly established and becomes a persistent maladaptive condition. The functional specialization of sensory neurons is driven by the expression of dedicated ion channel genes that fine-tune the membrane polarization to trigger and propagate action potentials in response to stimuli (Table 12).¹ Therefore, in simple terms the etiology of chronic pain can be described as the dysregulated expression of just a few genes in just a few neurons. However, to date, genetic treatments of chronic pain have not been successfully translated into human therapeutics.^{2,3}

In general, the types of chronic pain that occur in most parts of the body and the extremities involve afferent neurons of the dorsal root ganglia (DRG), which reside in clusters of nerve cells near the spinal cord and have long axons extending towards the skin, muscles, and organs, etc. (FIG. 1).^{1,4} The mechanism of enhanced excitability involves voltage-gated ion channels and background/leak channels that set the resting membrane potential and firing threshold of DRG neurons (Table 12).¹ Under normal conditions, chemical, mechanical, or thermal stimuli are required to activate receptors and ion channels in peripheral nerve endings to initiate action potentials that propagate along the axons of DRG neurons (FIG. 1). Finally, the dendritic termini of the DRG neurons liberate glutamate and substance-P at synapses in the spinal cord dorsal horn, activating second-order neurons that communicate pain signals to the brain.¹

Described herein is a novel approach to address chronic pain by deploying various genome-editing agents to modify the genes responsible for propagation of pain signals in dysregulated DRG neurons, by selectively targeting the local nerve endings at the sites of pain using neuron-specific viral vectors (FIG. 2). The genome editing reactions described herein can be achieved by any of the major types of genome editing tools: (i) base-editors that catalyze chemical reactions on nucleobases (e.g., cytidine deaminase-Cas9 fusion⁵); (ii) programmable nucleases with DNA cutting activity (e.g., WT Cas9,⁶⁻⁸ paired Cas9 nickases9 or Fok1-nuclease-dCas⁹ fusions,^{10,11} or compatible analogs such as Cpf1,¹² NgAgo,¹³ etc.); as well as (iii) TALENs, zinc-finger nucleases, etc.^{14,15} The best studied example of a neurotropic virus that has potential as a delivery vector for genome editing constructs is Herpes Simplex Virus 1 (HSV-1),¹⁶ which targets nerve endings in vivo and usurps retrograde axon transport to move the viral DNA up to the cell body of DRG neurons.¹⁷ Therefore, using a neurotropic virus as a delivery strategy enables genome editing treatments aimed at the sites of pain, despite the fact that the genetic material within the nuclei of DRG neurons is quite distant and indistinguishable from unrelated cells within each ganglion (FIG. 2).⁴ HSV-1 vectors have the additional advantage of packaging a large double-stranded DNA genome (>150 kbp),¹⁸ which can easily accommodate an expression construct for any programmable genome-editing enzyme (4-5 kbp),¹⁹ multiple guide-RNAs, and regulatory sequences. Constructs up to 30 to 40 kbp can be inserted by replacing non-essential genes of HSV-1,16 and alternatively a designed 100 kbp amplicon can be packaged into helper HSV-1 capsids.²⁰ Examples of other potentially useful neurotropic viruses include the broader herpesviridae group,²¹ varicella-zoster, pseudorabies, cytomegalovirus. Epstein-Barr viruses, encephalitis viruses, polio, coxsackie, echo, mumps, measles, and rabies viruses.^{22,23}

This genome editing treatment incorporates multiple design elements that achieve precise and selective targeting of genome editing agents to pain-causative neurons, arising from: 1) localized delivery of a non-replicative viral vector that requires synaptic terminals, sparing the bulk of somatic tissues near the pain site, 2) neuron-specific promoters that drive expression of the genome editing construct, and 3) guide-RNA programmed targeting of non-essential ion channel genes exclusively expressed by DRG neurons to spare other types of neurons (efferent neurons, interneurons, etc.). Safety can be further enhanced using high-specificity Cas9 nuclease variants^{9,10,24,25} and guide-RNAs without off-target matches in the human genome,^{26,27} as well as limiting the stability, activity, or expression of the construct, etc.^{28,29} By contrast, traditional pharmacological modulation of localized DRG neuron signaling would be challenging to achieve using small molecules or antibodies,^{30,31} because these spread systemically and must be optimized for selectivity among many similar ion channel isoforms.³² Therefore, such strategies are still in early development.³³ Pharmacological analgesics and opioids act rapidly but reversibly, while genome editing is essentially permanent, therefore, standard medications may be co-administered over the period required for the delivery vector and the expression construct to take action.

Human DRG neurons constitutively express specific and specialized ion channels that have been implicated in afferent pain signaling,1 and fulfill the required criteria as targets for modulation of chronic pain conditions. Three sodium channels (NaV1.7, NaV1.8, and NaV1.9) are constitutively expressed in DRG neurons, and a fourth gene (NaV1.3) displays elevated expression after nerve injury (Table 12).¹ Genetic evidence from spontaneous mutations of NaV1.7 (SCN9A) in humans.³⁴⁻³⁷ and animal models,³⁸ strongly suggests that the phenotypic outcome of gene ablation, loss-of-function, destabilization of the transcript and/or protein folding will be the eradication of pain transmission.³⁴⁻³⁶ without compromising the normal function of the DRG neurons in triggering action potentials reaching a normal membrane depolarization threshold (FIG. 1). Disruption of SCN9A is only desirable at a localized level, because nociception is essentially a protective mechanism from overextension and deformation for our joints and muscles,³⁴⁻³⁶ and it is also necessary for our sense of smell.³⁹ In the extreme, humans presenting homozygous SCN9A loss-of-function mutations present Congenital Insensitivity to Pain (CIP).³⁴⁻³⁶ Conversely, gain-of-function mutations in the sodium channels NaV1.7 (SCN9A) or NaV1.8 (SCN10A) cause congenital pain syndromes such as Primary Erythermalgia.³⁷ Moreover, the SCN9A gene is also involved in itching.^{30,40}

Guide sequences for programming the disruption of SCN9A gene using cytidine deaminase base-editors are shown in Tables 2, 4, and 6. Top scoring guide sequences are listed in Tables 7-9. Top-scoring guide-RNAs for Cas9 nuclease and paired nickases are shown in Table 10 and 11. Interestingly, gain-of-function mutations in the sodium channel NaV1.9 (SCN11A) are known to reduce pain transmission,^{38,41} which can be potentially replicated using cytosine deamination base-editors. These strategies can be extended to other ion channels expressed in DRG neurons (FIG. 3). Alternative target genes include the voltage-gated calcium channel CaV3.2 (CACNA1H),⁴² the calcium-activated chloride channel (ANO1),^{43,44} and the hyperpolarization-activated cation channels (HCN1 and HCN2) (Table 12).^{45,46} Moreover, these concepts can be further extended by implementing functional genomic screening of guide-RNA libraries in cell lines,^{47,48} towards the unbiased identification of new target genes and genomic locations that indirectly modulate the DRG neuron ion channels and other mechanisms.¹

In conclusion, chronic pain could be permanently suppressed with unprecedented anatomical precision by genome editing treatment of a small number of DRG neurons, which are the root cause of the condition. This new treatment exploits numerous design elements for specificity and safety, and in principle, can be curative. By engaging the distal axon projections of DRG neurons in the region of chronic pain, this approach is both specific and potentially generalizable to theoretically any location in the body to treat dysregulated neuronal firing established by countless sources of trauma, and regardless of the time that may have elapsed since the onset of chronic pain.

TABLE 12
Ion Channel Genes in DRG Neurons Responsible for Pain Propagation

| Channel | Gene | Channel type and | | Target validation |
|---|---|---|---|---|
| name | name | function | Expression evidence | evidence |
| Nav1.7 | SCN9A | Tetrodotoxin (TTX)- | Peripheral neuron specific. | Loss-of-function and |
| | | sensitive rapidly | Constitutive, and elevated | gain-of-function |
| | | inactivating sodium | after nerve injury | mutations |
| | | current | and inflammation | |
| Nav1.8 | SCN10A | TTX-resistant slowly | DRG specific. | Loss-of-function |
| | | inactivating sodium | Constitutive, and elevated | mutations |
| | | current | after nerve injury | |
| | | | and inflammation | |
| Nav1.9 | SCN11A | TTX-resistant | DRG specific. | Gain-of-function |
| | | persistent sodium | Constitutive, and elevated | mutations |
| | | current | after nerve injury | |
| | | | and inflammation | |
| Nav1.3 | SCN3A | TTX-sensitive rapidly | Elevated expression after | |
| | | inactivating sodium | axotomy and nerve injury | |
| | | current | | |
| Cav3.2 | CACNA1H | T-type calcium current | Constitutive, and elevated | KO mice and genetic |
| | | | after nerve injury | models |
| | | | and inflammation | |
| HCN1 | HCN1 | Hyperpolarization- | Constitutive, and elevated | |
| | | activated cation | after inflammation | |
| | | current | and chemotherapy | |
| HCN2 | HCN2 | Hyperpolarization- | Constitutive, and elevated | |
| | | activated cation | after inflammation | |
| | | current | | |
| Ano1 | ANO1 | Calcium-activated | Constitutive, and elevated | |
| | | chloride current | after nerve injury. | |

Human SCN9A primary protein and cDNA sequence alignment. Underlined: examples of residues and codons predicted to produce a premature stop codon, inactivation, loss-of-function, or destabilization of protein folding, as a result of base-editing using a cytidine deaminase-Cas9 construct.

```
(SEQ ID NO: 2434)
gaggagctgaagaggaattaaaatatacaggatgaaaagatggcaatgttgcctccccca
                                      .. M A M L P P P
ggacctcagagctttgtccatttcacaaaacagtctcttgccctcattgaacaacgcatt

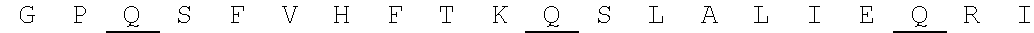

gctgaaagaaaatcaaaggaacccaaagaagaaaagaaagatgatgatgaagaagcccca
  A E P K S K E P K E E K K D D D E E A P
aagccaagcagtgacttggaagctggcaaacagctgcccttcatctatggggacattcct

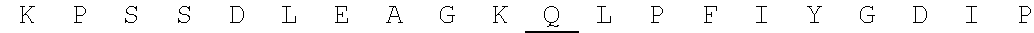

cccggcatggtgtcagagcccctggaggacttggacccctactatgcagacaaaaagact
  P G M V S E P L E D L D P Y Y A D K K I
ttcatagtattgaacaaagggaaaacaatcttccgtttcaatgccacacctgctttatat
  F I V L N K G K T I F R F N A I P A L Y
atgctttctcctttcagtcctctaagaagaatatctattaagattttagtacactcctta
  M L S P F S P L P R I S I K I L V H S L
ttcagcatgctcatcatgtgcactattctgacaaactgcatatttatgaccatgaataac

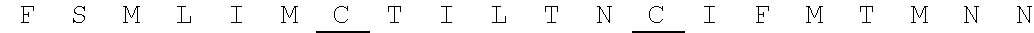

ccaccggactggaccaaaaatgtcgagtacacttttactggaatatatacttttgaatca

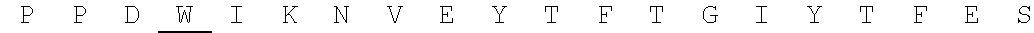

cttgtaaaaatccttgcaagaggcttctgtgtaggagaattcacttttcttcgtgacccg

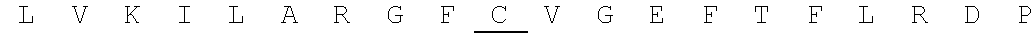

tggaactggctggattttgtcgtcattgtttttgagtatttaacagaatttgtaaaccta

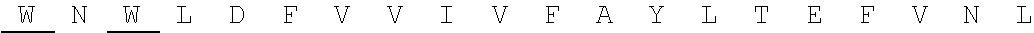

ggcaatgtttcagctcttcgaactttcagagtattgagagctttgaaaactatttctgta
  G N V S A L P T F R V L R A L K T I S V
atcccaggcctgaagacaattgtaggggctttgatccagtcagtgaagaagctttctgat

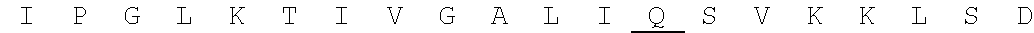

gtcatgatcctgactgtgttctgtctgagtgtgtttgcactaattggactacagctgttc

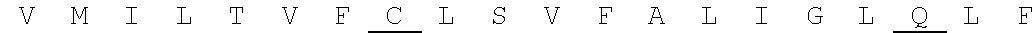

atgggaaacctgaagcataaatgttttcgaaattcacttgaaaataatgaaacattagaa

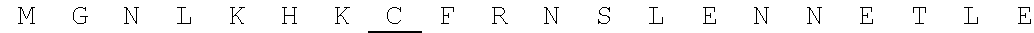

agcataatgaataccctagagagtgaagaagactttagaaaatatttttattacttggaa
  S I M N T L E S E E D F R K Y F Y Y L E
ggatccaaagatgctctcctttgtggtttcagcacagattcaggtcagtgtccagagggg

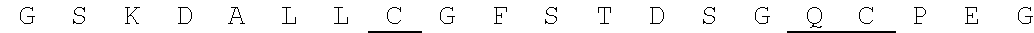

tacacctgtgtgaaaattggcagaaaccctgattatggctacacgagctttgacactttc

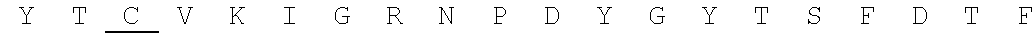

agctgggccttcttagccttgtttaggctaatgacccaagattactgggaaaacctttac

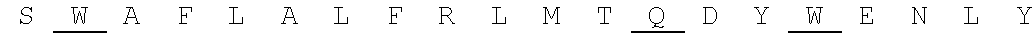

caacagacgctgcgtgctgctggcaaaacctacatgatcttctttgtcgtagtgattttc

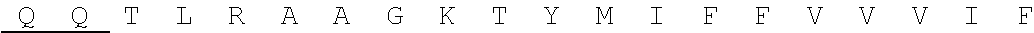

ctgggctccttttatctaataaacttgatcctggctgtggttgccatggcatatgaagaa
  L G S F Y L I N L I L A V V A M A Y E E
cagaaccaggcaaacattgaagaagctaaacagaaagaattagaatttcaacagatgtta

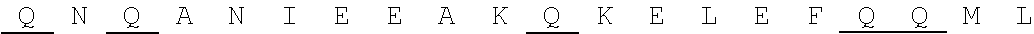

gaccgtcttaaaaaagagcaagaagaagctgaggcaattgcagcggcagcggctgaatat

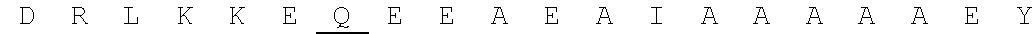

acaagtattaggagaagcagaattatgggcctctcagagagttcttctgaaacatccaaa
  T S I R R S R I M G L S E S S S E T S K
ctgagctctaaaagtgctaaagaaagaagaaacagaagaaagaaaaagaatcaaaagaag

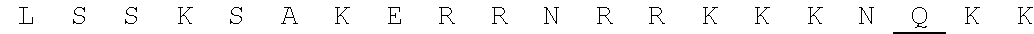

ctctccagtggagaggaaaagggagatgctgagaaattgtcgaaatcagaatcagaggac
  L S S G E E K G D A E K L S K S E S E D
agcatcagaagaaaaagtttccaccttggtgtcgaagggcataggcgagcacatgaaaag
  S I P R K S F H L G V E G H R R A H E K
aggttgtctacccccaatcagtcaccactcagcattcgtggctccttgttttctgcaagg

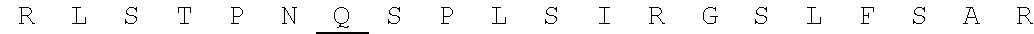

cgaagcagcagaacaagtctttttagtttcaaaggcagaggaagagatataggatctgag
  P S S R T S L F S P K G R G P D I G S E
actgaatttgccgatgatgagcacagcatttttggagacaatgagagcagaaggggctca
  T E F A D D E H S I F G D N E S R R G S
ctgtttgtgccccacagaccccaggagcgacgcagcagtaacatcagccaagccagtagg

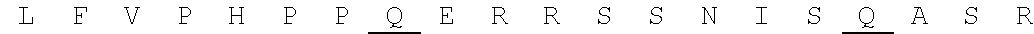

tccccaccaatgctgccggtgaacgggaaaatgcacagtgctgtggactgcaacggtgtg

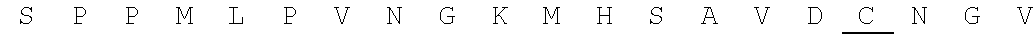

gtctccctggttgatggacgctcagccctcatgctccccaatggacagcttctgccagag

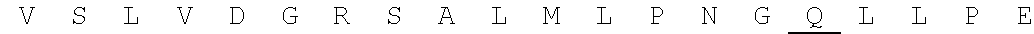

ggcacgaccaatcaaatacacaagaaaaggcgttgtagttcctatctcctttcagaggat

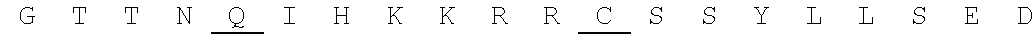

atgctgaatgatcccaacctcagacagagagcaatgagtagagcaagcatattaacaaac

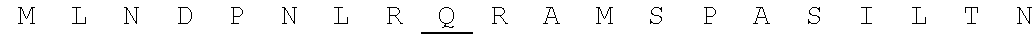

actgtggaagaacttgaagagtccagacaaaaatgtccaccttggtggtacagatttgca

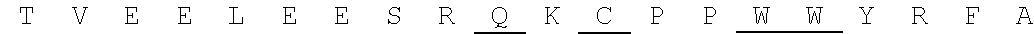

cacaaattcttgatctggaattgctctccatattggataaaattcaaaaagtgtatctat

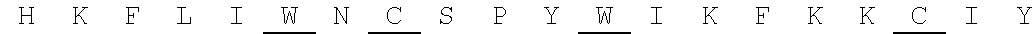

tttattgtaatggatccttttgtagatcttgcaattaccatttgcatagttttaaacaca

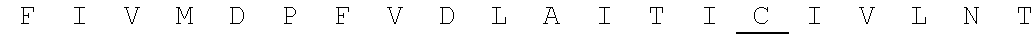

ttatttatggctatggaacaccacccaatgactgaggaattcaaaaatgtacttgctata
  L F M A M E H H P M T E E F K N V L A I
ggaaatttggtctttactggaatctttgcagctgaaatggtattaaaactgattgccatg
  G N L V F T G I F A A E M V L K L I A M
gatccatatgagtatttccaagtaggctggaatatttttgacagccttattgtgacttta

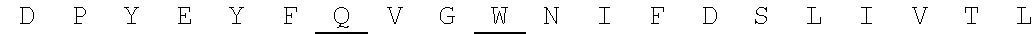

agtttagtggagctctttctagcagatgtggaaggattgtcagttctgcgatcattcaga
  S L V K L F L A D V E G L S V L P S F K
ctgctccgagtcttcaagctggcaaaatcctggccaacattgaacatgctgattaagatc

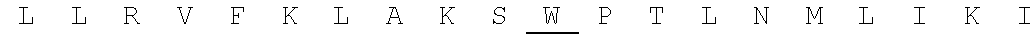

attggtaactcagtaggggctctaggtaacctcaccttagtgttggccatcatcgtcttc
  I G N S V G A L G N L T L V L A I I V F
atttttgctgtggtcggcatgcagctctttggtaagagctacaaagaatgtgtctgcaag

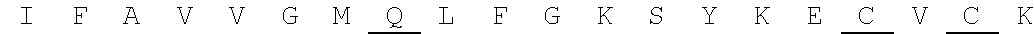

atcaatgatgactgtacgctcccacggtggcacatgaacgacttcttccactccttcctg

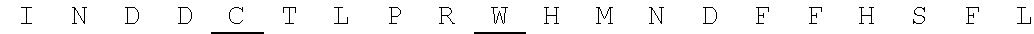

attgtgttccgcgtgctgtgtggagagtggatagagaccatgtgggactgtatggaggtc

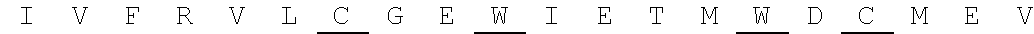

gctggtcaagctatgtgccttattgtttacatgatggtcatggtcattggaaacctggtg

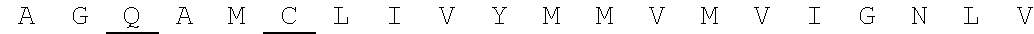

gtcctaaacctatttctggccttattattgagctcatttagttcagacaatcttacagca
  V L N L F L A L L L S S F S S D N L T A
attgaagaagaccctgatgcaaacaacctccagattgcagtgactagaattaaaaaggga

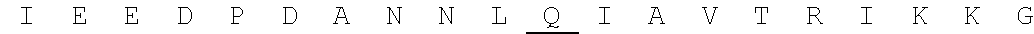

ataaattatgtgaaacaaaccttacgtgaatttattctaaaagcattttccaaaaagcca

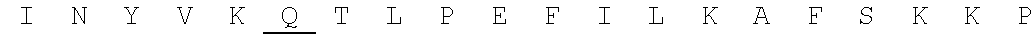

aagatttccagggagataagacaagcagaagatctgaatactaagaaggaaaactatatt

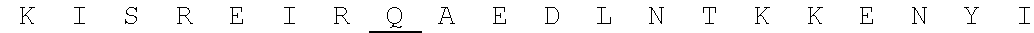

tctaaccatacacttgctgaaatgagcaaaggtcacaatttcctcaaggaaaaagataaa
  S N H T L A E M S K G H N F L K E K D K
atcagtggttttggaagcagcgtggacaaacacttgatggaagacagtgatggtcaatca

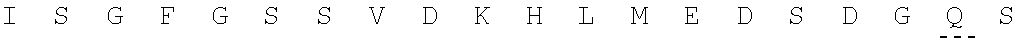

tttattcacaatcccagcctcacagtgacagtgccaattgcacctggggaatccgatttg
  F I H N P S L I V I V P I A P G E S D L
gaaaatatgaatgctgaggaacttagcagtgattcggatagtgaatacagcaaagtgaga
  E N M N A E E L S S D S D S E Y S K V R
ttaaaccggtcaagctcctcagagtgcagcacagttgataaccctttgcctggagaagga

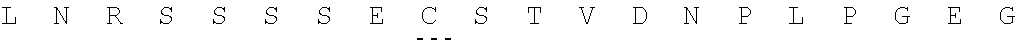

gaagaagcagaggctgaacctatgaattccgatgagccagaggcctgtttcacagatggt

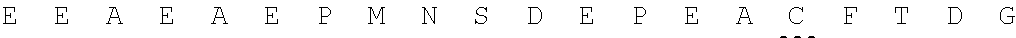

tgtgtatggaggttctcatgctgccaagttaacatagagtcagggaaaggaaaaatctgg

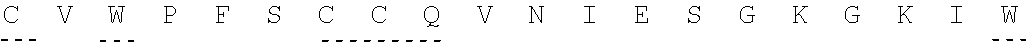

tggaacatcaggaaaacctgctacaagattgttgaacacagttggtttgaaagcttcatt

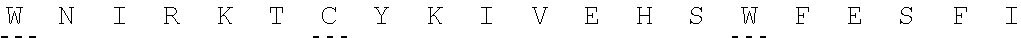

gtcctcatgatcctgctcagcagtggtgccctggcttttgaagatatttatattgaaagg
  V L M I L L S S G A L A F E D I Y I E R
aaaaagaccattaagattatcctggagtatgcagacaagatcttcacttacatcttcatt
  K K T I K I I L E Y A D E I F I Y I F I
ctggaaatgcttctaaaatggatagcatatggttataaaacatatttcaccaatgcctgg

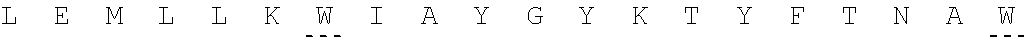

tgttggctggatttcctaattgttgatgtttctttggttactttagtggcaaacactctt

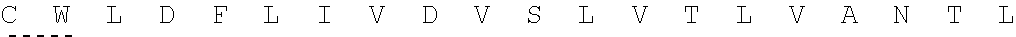

ggctactcagatcttggccccattaaatcccttcggacactgagagctttaagacctcta
  G Y S D L G P I K S L R I L P A L R P L
agagccttatctagatttgaaggaatgagggtcgttgtgaatgcactcataggagcaatt
  R A L S P F E G M R V V V N A L I G A I
ccttccatcatgaatgtgctacttgtgtgtcttatattctggctgatattcagcatcatg

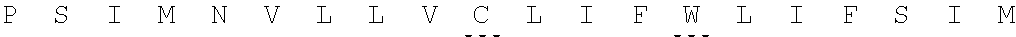

ggagtaaatttgtttgctggcaagttctatgagtgtattaacaccacagatgggtcacgg

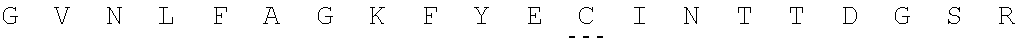

tttcctgcaagtcaagttccaaatcgttccgaatgttttgcccttatgaatgttagtcaa

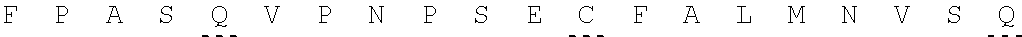

aatgtgagatggaaaaacctgaaagtgaactttgataatgtcggacttggttacctatct

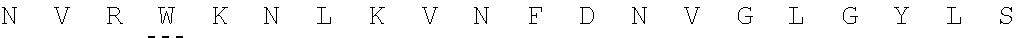

ctgcttcaagttgcaacttttaagggatggacgattattatgtatgcagcagtggattct

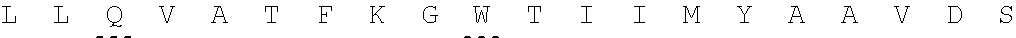

gttaatgtagacaagcagcccaaatatgaatatagcctctacatgtatatttattttgtc

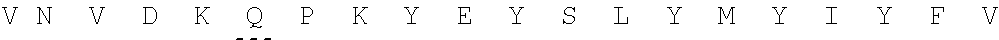

gtctttatcatctttgggtcattcttcactttgaacttgttcattggtgtcatcatagat
  V F I I F G S F F T L N L F I G V I I D
aatttcaaccaacagaaaaagaagcttggaggtcaagacatctttatgacagaagaacag

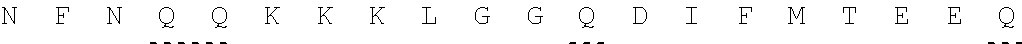

aagaaatactataatgcaatgaaaaagctggggtccaagaagccacaaaagccaattcct

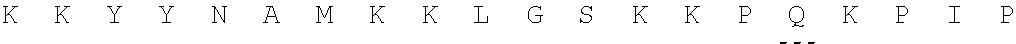

cgaccagggaacaaaatccaaggatgtatatttgacctagtgacaaatcaagcctttgat

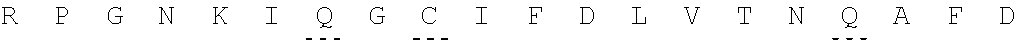

attagtatcatggttcttatctgtctcaacatggtaaccatgatggtagaaaaggagggt

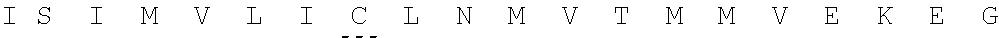

caaagtcaacatatgactgaagttttatattggataaatgtggtttttataatccttttc

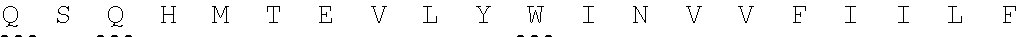

actggagaatgtgtgctaaaactgatctccctcagacactactacttcactgtaggatgg

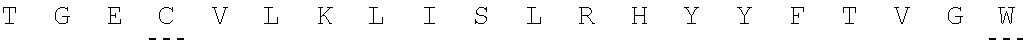

aatatttttgattttgtggttgtgattatctccattgtaggtatgtttctagctgatttg
  N I F D F V V V I I S I V G M F L A D L
attgaaacgtattttgtgtcccctaccctgttccgagtgatccgtcttgccaggattggc
  I E T Y F V S P T L F P V I R L A R I G
cgaatcctacgtctagtcaaaggagcaaaggggatccgcacgctgctctttgctttgatg
  R I L R L V K G A K G I R I L L F A L M
atgtcccttcctgcgttgtttaacatcggcctcctgctcttcctggtcatgttcatctac
  M S L P A L F N I G L L L F L V M F I Y
gccatctttggaatgtccaactttgcctatgttaaaaaggaagatggaattaatgacatg
  A I F G M S N F A Y V K K E D G I N D M
ttcaattttgagacctttggcaacagtatgatttgcctgttccaaattacaacctctgct

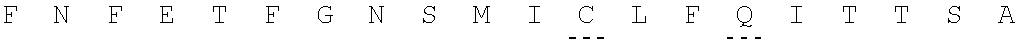

ggctgggatggattgctagcacctattcttaacagtaagccacccgactgtgacccaaaa

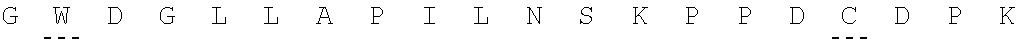

aaagttcatcctggaagttcagttgaaggagactgtggtaacccatctgttggaatattc

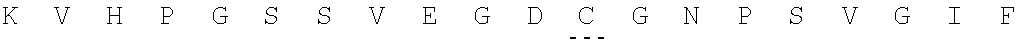

tactttgttagttatatcatcatatccttcctggttgtggtgaacatgtacattgcagtc
  Y F V S Y I I I S F L V V V N M Y I A V
atactggagaattttagtgttgccactgaagaaagtactgaacctctgagtgaggatgac
  I L E N F S V A T E E S T E P L S E D S
tttgagatgttctatgaggtttgggagaagtttgatcccgatgcgacccagtttatagag

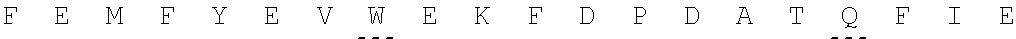

ttctctaaactctctgattttgcagctgccctggatcctcctcttctcatagcaaaaccc
  P S K L S D F A A A L D P P L L I A K P
aacaaagtccagctcattgccatggatctgcccatggttagtggtgaccggatccattgt

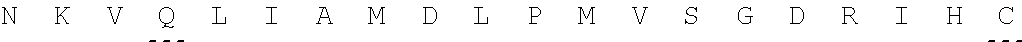

cttgacatcttatttgcttttacaaagcgtgttttgggtgagagtggggagatggattct
  L D I L F A F T K R V L G E S G E M D S
cttcgttcacagatggaagaaaggttcatgtctgcaaatccttccaaagtgtcctatgaa

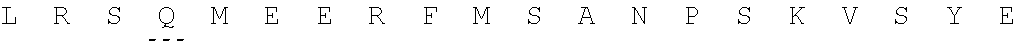

cccatcacaaccacactaaaacggaaacaagaggatgtgtctgctactgtcattcagcgt

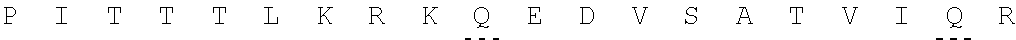

gcttatagacgttaccgcttaaggcaaaatgtcaaaaatatatcaagtatatacataaaa

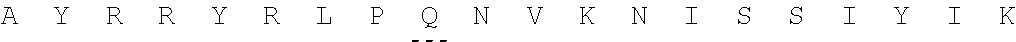

gatggagacagagatgatgatttactcaataaaaaagatatggcttttgataatgttaat
  D G D R D D D L L N K K D M A F D N V N
gagaactcaagtccagaaaaaacagatgccacttcatccaccacctctccaccttcatat
  E N S S P E K T D A T S S T T S P P S Y
gatagtgtaacaaagccagacaaagagaaatatgaacaagacagaacagaaaaggaagac
  D S V T K P D K E K Y E Q D R T E K E D
aaagggaaagacagcaaggaaagcaaaaaatagagcttcatttttgatatattgtttaca
  K G K D S K E S K K -
```

Human SCN9A gene sequence. Includes open reading frames (capitalized) and introns (lowercase, abridged). Underlined bases are predicted to disrupt the splicing of the RNA transcript, leading to diminished expression of functional protein. The start codon is also highlighted in bold.

(SEQ ID NO: 2435)
CGGGGCTGCTACCTCCACGGGCGCGCCCTGGCAGGAGGGGCGCAGTCTGC
TTGCAGGCGGTCGCCAGCGCTCCAGCGGCGGCTGTCGGCTTTCCAATTCC
GCCAGCTCGGCTGAGGCTGGGCTAGCCTGGGTGCCAGTGGCTGCTAGCGG
CAGGCGTCCCCTGAGCAACAGGAGCCCAGAGAAAAAGAAGCAGCCCTGAG
AGAGCGCCGGGGAAGGAGAGGCCCGCGCCCTCTCCTGGAGCCAGATTCTG

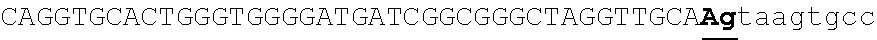

ttttcttttgctgcttctgtggggaggggaggagaagccctcggtctttc
. . . intron 1 . . .
tttatgttgttattattagtttttaatgggcctttcttggcaggcaaata

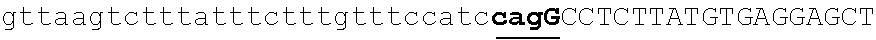

GAAGAGGAATTAAAATATACAGGATGAAAAGATGGCAATGTTGCCTCCCC
CAGGACCTCAGAGCTTTGTCCATTTCACAAAACAGTCTCTTGCCCTCATT
GAACAACGCATTGCTGAAAGAAAATCAAAGGAACCCAAAGAAGAAAAGAA
AGATGATGATGAAGAAGCCCCAAAGCCAAGCAGTGACTTGGAAGCTGGCA
AACAGCTGCCCTTCATCTATGGGGACATTCCTCCCGGCATGGTGTCAGAG

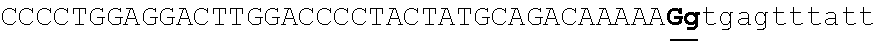

ttgacttcagtggtcagtttctgttggcttccttctgtataaaaattatt
. . . intron 2 . . .
atattgatgtgaaaaattgatattttggattctcaatttcatcctttctt

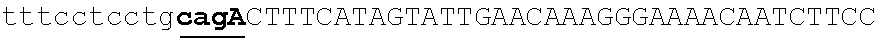

GTTTCAATGCCACACCTGCTTTATATATGCTTTCTCCTTTCAGTCCTCTA

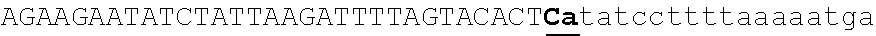

ttacatccagtggcactttatggtgtaatttttgctattttattcaaata
. . . intron 3 . . .
cccactgtcgtctcttttgttccttgattctaagctacCTTATTCAGCAT
GCTCATCATGTGCACTATTCTGACAAACTGCATATTTATGACCATGAATA

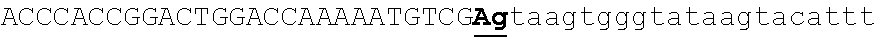

taatatagttttggtattatcatttcatcctttccttttcctgccaggaa
. . . intron 4 . . .
ttataaagatttacatggtggttgtattcttttcacatctagtatcccaa
tggaatcttgtgtttagGTACACTTTTACTGGAATATATACTTTTGAATC
ACTTGTAAAAATCCTTGCAAGAGGCTTCTGTGTAGGAGAATTCACTTTTC

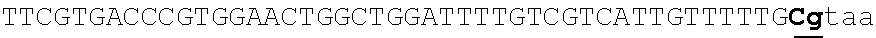

gtactttcagctttttgaaacggcaaatttatgaaaatctcaggcagcac
. . . intron 5 . . .
tcaggtaagtatcatagactctatctaaattctgaataattctgatttaa

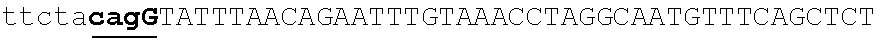

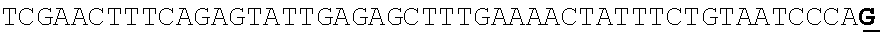

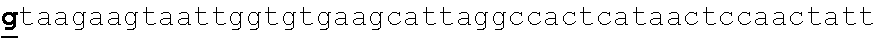

. . . intron 6 . . .

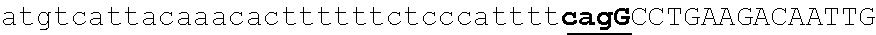

TAGGGGCTTTGATCCAGTCAGTGAAGAAGCTTTCTGATGTCATGATCCTG
ACTGTGTTCTGTCTGAGTGTGTTTGCACTAATTGGACTACAGCTGTTCAT
GGGAAACCTGAAGCATAAATGTTTTCGAAATTCACTTGAAAATAATGAAA

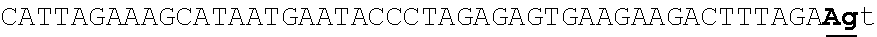

aagaatgtccttgcatttgttattaggttgaaataatgctaaaaacattg
. . . intron 7 . . .
attaatttacctcctttatcacaatcacagattaaagtctgtgatgttat

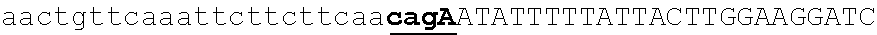

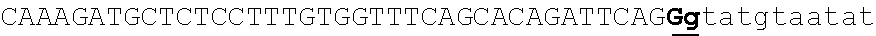

ttgttttctttttagtctaaaggctgaaagagaaggaaaagaatgttcag
. . . intron 8 . . .
cagtaaggctatttagcttgtgtcctgaagacactctcacctataatgtt

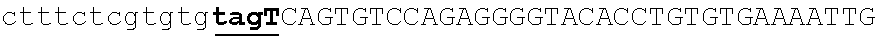

GCAGAAACCCTGATTATGGCTACACGAGCTTTGACACTTTCAGCTGGGCC
TTCTTAGCCTTGTTTAGGCTAATGACCCAAGATTACTGGGAAAACCTTTA

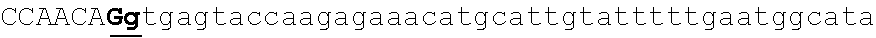

. . . intron 9 . . .
tgtacctggtgtatgttaagagcctgtattaggaggttttttatttattt

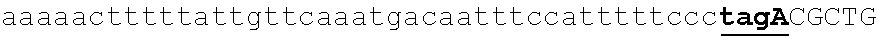

CGTGCTGCTGGCAAAACCTACATGATCTTCTTTGTCGTAGTGATTTTCCT
GGGCTCCTTTTATCTAATAAACTTGATCCTGGCTGTGGTTGCCATGGCAT
ATGAAGAACAGAACCAGGCAAACATTGAAGAAGCTAAACAGAAAGAATTA
GAATTTCAACAGATGTTAGACCGTCTTAAAAAAGAGCAAGAAGAAGCTGA

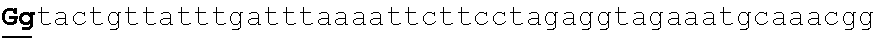

. . . intron 10 . . .
tgtcctagggtttcctaggatttggaaatgactcatttaagtgttaacgt

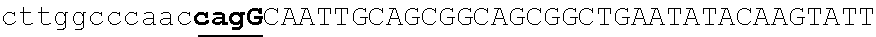

AGGAGAAGCAGAATTATGGGCCTCTCAGAGAGTTCTTCTGAAACATCCAA
ACTGAGCTCTAAAAGTGCTAAAGAAAGAAGAAACAGAAGAAAGAAAAAGA
ATCAAAAGAAGCTCTCCAGTGGAGAGGAAAAGGGAGATGCTGAGAAATTG
TCGAAATCAGAATCAGAGGACAGCATCAGAAGAAAAAGTTTCCACCTTGG
TGTCGAAGGGCATAGGCGAGCACATGAAAAGAGGTTGTCTACCCCCAATC

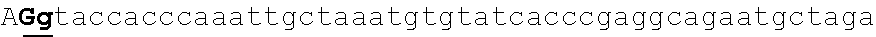

. . . intron 11 . . .
atatgaagtgtacttctatcagtaggtgcttcagcaaccacgtttttttt

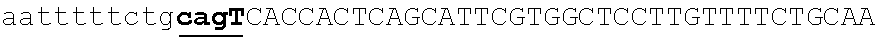

GGCGAAGCAGCAGAACAAGTCTTTTTAGTTTCAAAGGCAGAGGAAGAGAT
ATAGGATCTGAGACTGAATTTGCCGATGATGAGCACAGCATTTTTGGAGA
CAATGAGAGCAGAAGGGGCTCACTGTTTGTGCCCCACAGACCCCAGGAGC
GACGCAGCAGTAACATCAGCCAAGCCAGTAGGTCCCCACCAATGCTGCCG
GTGAACGGGAAAATGCACAGTGCTGTGGACTGCAACGGTGTGGTCTCCCT
GGTTGATGGACGCTCAGCCCTCATGCTCCCCAATGGACAGCTTCTGCCAG

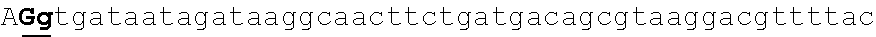

ctatataagcaagattttatcttatacctacaatttattaggattctgtt
. . . intron 12 . . .

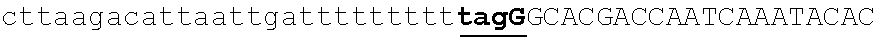

AAGAAAAGGCGTTGTAGTTCCTATCTCCTTTCAGAGGATATGCTGAATGA
TCCCAACCTCAGACAGAGAGCAATGAGTAGAGCAAGCATATTAACAAACA

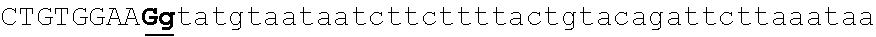

. . . intron 13 . . .

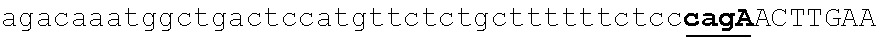

GAGTCCAGACAAAAATGTCCACCTTGGTGGTACAGATTTGCACACAAATT
CTTGATCTGGAATTGCTCTCCATATTGGATAAAATTCAAAAAGTGTATCT
ATTTTATTGTAATGGATCCTTTTGTAGATCTTGCAATTACCATTTGCATA
GTTTTAAACACATTATTTATGGCTATGGAACACCACCCAATGACTGAGGA

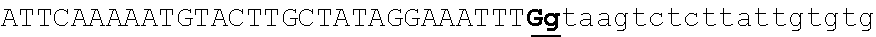

ttatgtactcatagtttctctttttagttgtcatcattgtcatttcatat
. . . intron 14 . . .
aacagtgattattatcattgtgttgatttcctgttttctaatattaacag

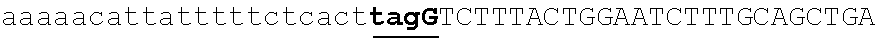

AATGGTATTAAAACTGATTGCCATGGATCCATATGAGTATTTCCAAGTAG
GCTGGAATATTTTTGACAGCCTTATTGTGACTTTAAGTTTAGTGGAGCTC

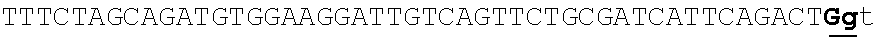

aaacataaactaaggttgccattatattctataataaaggggtatttctt
. . . intron 15 . . .

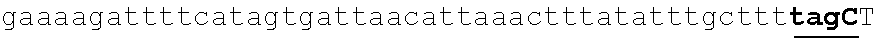

CCGAGTCTTCAAGTTGGCAAAATCCTGGCCAACATTGAACATGCTGATTA
AGATCATTGGTAACTCAGTAGGGGCTCTAGGTAACCTCACCTTAGTGTTG
GCCATCATCGTCTTCATTTTTGCTGTGGTCGGCATGCAGCTCTTTGGTAA
GAGCTACAAAGAATGTGTCTGCAAGATCAATGATGACTGTACGCTCCCAC
GGTGGCACATGAACGACTTCTTCCACTCCTTCCTGATTGTGTTCCGCGTG
CTGTGTGGAGAGTGGATAGAGACCATGTGGGACTGTATGGAGGTCGCTGG
TCAAGCTATGTGCCTTATTGTTTACATGATGGTCATGGTCATTGGAAACC

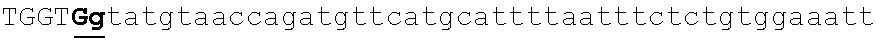

. . . intron 16 . . .
attttattttttatatttcctgtctccctatttctctaccccctctcccc

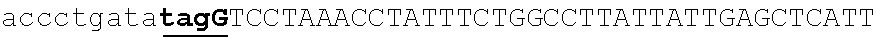

TAGTTCAGACAATCTTACAGCAATTGAAGAAGACCCTGATGCAAACAACC
TCCAGATTGCAGTGACTAGAATTAAAAAGGGAATAAATTATGTGAAACAA
ACCTTACGTGAATTTATTCTAAAAGCATTTTCCAAAAAGCCAAAGATTTC
CAGGGAGATAAGACAAGCAGAAGATCTGAATACTAAGAAGGAAAACTATA
TTTCTAACCATACACTTGCTGAAATGAGCAAAGGTCACAATTTCCTCAAG
GAAAAAGATAAAATCAGTGGTTTTGGAAGCAGCGTGGACAAACACTTGAT
GGAAGACAGTGATGGTCAATCATTTATTCACAATCCCAGCCTCACAGTGA
CAGTGCCAATTGCACCTGGGGAATCCGATTTGGAAAATATGAATGCTGAG

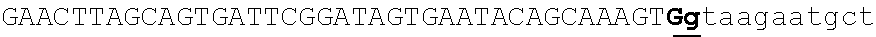

tcatatactttgtgtttcatattaacaattagtatgaaatgaatgaaaat
. . . intron 17 . . .
tttgaatgaactctaaatgaactacctggtggggtggtgaattcctttct
agAGATTAAACCGGTCAAGCTCCTCAGAGTGCAGCACAGTTGATAACCCT
TTGCCTGGAGAAGGAGAAGAAGCAGAGGCTGAACCTATGAATTCCGATGA

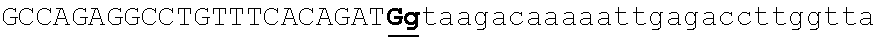

gcattccttaattagtgttetggggtttgtcttaacgcctaatacttacc
. . . intron 18 . . .

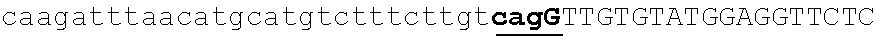

ATGCTGCCAAGTTAACATAGAGTCAGGGAAAGGAAAAATCTGGTGGAACA
TCAGGAAAACCTGCTACAAGATTGTTGAACACAGTTGGTTTGAAAGCTTC

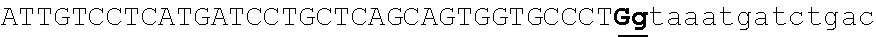

acctaagtcaatatattgattaagtcaatattctttaaaatgagctaaaa
. . . intron 19 . . .
tcctgttttttttaaatgaatcatgaagcttaagttgtgcatgattgaaa

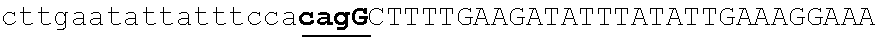

AAGACCATTAAGATTATCCTGGAGTATGCAGACAAGATCTTCACTTACAT
CTTCATTCTGGAAATGCTTCTAAAATGGATAGCATATGGTTATAAAACAT
ATTTCACCAATGCCTGGTGTTGGCTGGATTTCCTAATTGTTGATgtaggt
acttttgagtacattttaaaagaggatttattcttactgtgtgttgtgaa
. . . intron 20 . . .
agtttcagaattgactttttccttttatgcttcatcattttattgacaca

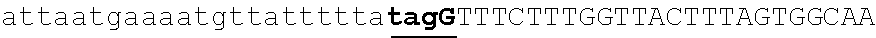

ACACTCTTGGCTACTCAGATCTTGGCCCCATTAAATCCCTTCGGACACTG

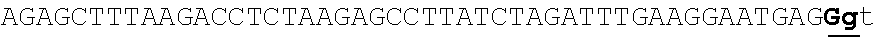

aagaaaatactaaactttataatgttcttattttttaatggggtttaaaa
. . . intron 21 . . .
ctttcatgttgcctatttaacatcttactaatcctaatcatgcttttctt

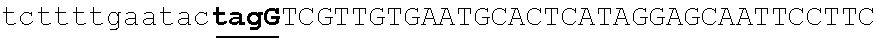

CATCATGAATGTGCTACTTGTGTGTCTTATATTCTGGCTGATATTCAGCA
TCATGGGAGTAAATTTGTTTGCTGGCAAGTTCTATGAGTGTATTAACACC
ACAGATGGGTCACGGTTTCCTGCAAGTCAAGTTCCAAATCGTTCCGAATG
TTTTGCCCTTATGAATGTTAGTCAAAATGTGCGATGGAAAAACCTGAAAG

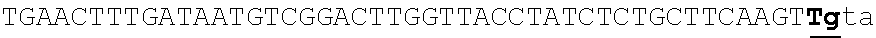

agtgtcccatttcatgagtgcttggtattttaatagatattggacgaagg
. . . intron 22 . . .
tctgtttatggctattttagaatatgagcttaacattcaaattctattaa

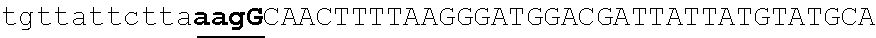

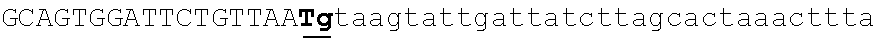

tttttaaaagcttcttagtttatttcagtgatttccaaactataacttca
. . . intron 23 . . .

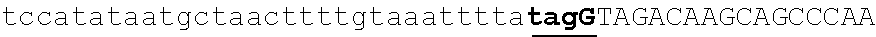

ATATGAATATAGCCTCTACATGTATATTTATTTTGTCGTCTTTATCATCT
TTGGGTCATTCTTCACTTTGAACTTGTTCATTGGTGTCATCATAGATAAT

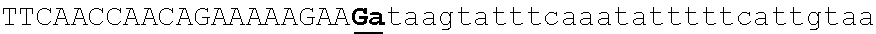

. . . intron 24 . . .
tttagtaatctatagaaagatgtagacaatgattctggttttaactacat
ttattttttgtttgtttctttacCTTGGAGGTCAAGACATCTTTATGACA
GAAGAACAGAAGAAATACTATAATGCAATGAAAAAGCTGGGGTCCAAGAA

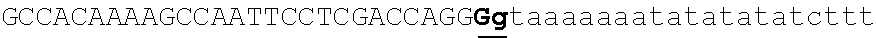

agcatatagattttcaaattatttctaattcatttttaatgcacatcttt
. . . intron 25 . . .
aatttctggataatacttgaaaagtttactctgcattegatattattctt

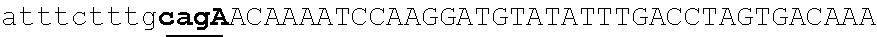

TCAAGCCTTTGATATTAGTATCATGGTTCTTATCTGTCTCAACATGGTAA
CCATGATGGTAGAAAAGGAGGGTCAAAGTCAACATATGACTGAAGTTTTA
TATTGGATAAATGTGGTTTTTATAATCCTTTTCACTGGAGAATGTGTGCT
AAAACTGATCTCCCTCAGACACTACTACTTCACTGTAGGATGGAATATTT

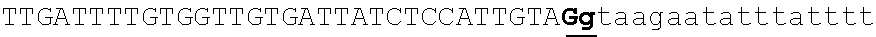

tcagattttattttttgagtaaagctaaacttcacttatgctcaaggaag
. . . intron 26 . . .
ctgtttagagtcatcatttcaggtagcatacatctttaaatattttattt

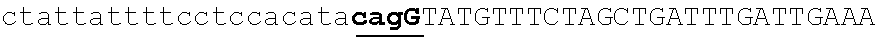

CGTATTTTGTGTCCCCTACCCTGTTCCGAGTGATCCGTCTTGCCAGGATT
GGCCGAATCCTACGTCTAGTCAAAGGAGCAAAGGGGATCCGCACGCTGCT
CTTTGCTTTGATGATGTCCCTTCCTGCGTTGTTTAACATCGGCCTCCTGC
TCTTCCTGGTCATGTTCATCTACGCCATCTTTGGAATGTCCAACTTTGCC
TATGTTAAAAAGGAAGATGGAATTAATGACATGTTCAATTTTGAGACCTT
TGGCAACAGTATGATTTGCCTGTTCCAAATTACAACCTCTGCTGGCTGGG
ATGGATTGCTAGCACCTATTCTTAACAGTAAGCCACCCGACTGTGACCCA
AAAAAAGTTCATCCTGGAAGTTCAGTTGAAGGAGACTGTGGTAACCCATC
TGTTGGAATATTCTACTTTGTTAGTTATATCATCATATCCTTCCTGGTTG
TGGTGAACATGTACATTGCAGTCATACTGGAGAATTTTAGTGTTGCCACT
GAAGAAAGTACTGAACCTCTGAGTGAGGATGACTTTGAGATGTTCTATGA
GGTTTGGGAGAAGTTTGATCCCGATGCGACCCAGTTTATAGAGTTCTCTA
AACTCTCTGATTTTGCAGCTGCCCTGGATCCTCCTCTTCTCATAGCAAAA
CCCAACAAAGTCCAGCTCATTGCCATGGATCTGCCCATGGTTAGTGGTGA
CCGGATCCATTGTCTTGACATCTTATTTGCTTTTACAAAGCGTGTTTTGG
GTGAGAGTGGGGAGATGGATTCTCTTCGTTCACAGATGGAAGAAAGGTTC
ATGTCTGCAAATCCTTCCAAAGTGTCCTATGAACCCATCACAACCACACT
AAAACGGAAACAAGAGGATGTGTCTGCTACTGTCATTCAGCGTGCTTATA
GACGTTACCGCTTAAGGCAAAATGTCAAAAATATATCAAGTATATACATA
AAAGATGGAGACAGAGATGATGATTTACTCAATAAAAAAGATATGGCTTT
TGATAATGTTAATGAGAACTCAAGTCCAGAAAAAACAGATGCCACTTCAT
CCACCACCTCTCCACCTTCATATGATAGTGTAACAAAGCCAGACAAAGAG
AAATATGAACAAGACAGAACAGAAAAGGAAGACAAAGGGAAAGACAGCAA
GGAAAGCAAAAAATAGAGCTTCATTTTTGATATATTGTTTACAGCCTGTG
AAAGTGATTTATTTGTGTTAATAAAACTCTTTTGAGGAAGTCTATGCCAA
AATCCTTTTTATCAAAATATTCTCGAAGGCAGTGCAGTCACTAACTCTGA
TTTCCTAAGAAAGGTGGGCAGCATTAGCAGATGGTTATTTTTGCACTGAT
GATTCTTTAAGAATCGTAAGAGAACTCTGTAGGAATTATTGATTATAGCA
TACAAAAGTGATTCAGTTTTTTGGTTTTTAATAAATCAGAAGACCATGTA
GAAAACTTTTACATCTGCCTTGTCATCTTTTCACAGGATTGTAATTAGTC
TTGTTTCCCATGTAAATAAACAACACACGCATACAGAAAAATCTATTATT
TATCTATTATTTGGAAATCAACAAAAGTATTTGCCTTGGCTTTGCAATGA
AATGCTTGATAGAAGTAATGGACATTAGTTATGAATGTTTAGTTAAAATG
CATTATTAGGGAGCTTGACTTTTTATCAATGTACAGAGGTTATTCTATAT
TTTGAGGTGCTTAAATTTATTCTACATTGCATCAGAACCAATTTATATGT
GCCTATAAAATGCCATGGGATTAAAAATATATGTAGGCTATTCATTTCTA
CAAATGTTTTTCATTCATCTTGACTCACATGCCAACAAGGATAAGACTTA
CCTTTAGAGTATTGTGTTTCATAGCCTTTCTTCTTTCATATCCCTTTTTG
TTCATAGAATAACCACAGAACTTGAAAAATTATTCTAAGTACATATTACA
CTCCTCAAAAAAAACAAAGATAACTGAGAAAAAAGTTATTGACAGAAGTT
CTATTTGCTATTATTTACATAGCCTAACATTTGACTGTGCTGCCCAAAAT
ACTGATAATAGTCTCTTAAACTCTTTTGTCAAATTTTCCTGCTTTCTTAT
GCAGTATTGTTTAGTCATCCTTTCGCTGTAAGCAAAGTTGATGAAATCCT
TCCTGATATGCAGTTAGTTGTTTGACCACGGTACATACTTGAGCAGATAA
TAACTTGGGCACAGTATTTATTGCATCACTTGTATACAATCCCGTGTTTG
GCAAGCTTTCAAATCATGTAATATGACAGACTTTACACAGATATGTGTTT
AGTATGAATAAAAAAGCATTGAAATAGGGATTCTTGCCAACTTGCTCTCT
TGCCACCAACTTACTTTCCTAAATTATGGAAGTAATCTTTTTTGGATATA
CTTCAATGTATACAATGAGGAAGATGTCACCTTCTCCTTAAAATTCTATG
ATGTGAAATATATTTTGCCTCAATCAACACAGTACCATGGGCTTCTAATT
TATCAAGCACATATTCATTTTGCATTAGCTGTAGACATCTAGTTTTTTGA
AAACACCTATTAATAGTAATTTGAAAAGAAATAACCATAATGCTTTTTTT
CGTGAGTTTATTTCAGGAATATGAGATCTTTCTTCTATAAAGTTATTCAT
GCACAGGCAAAAATTGAGCTACACAGGTAGAATGTAGTTTTACTTAGAAG
ATTTTTGTGGGAGGTTTTGAAGCAAATATATAAAACAACTTTCACTAATT
TGCTTTCCATATTTAAAAAATAATAAATTACATTTATATAATAAATGTTT
AAAGCACATATTTTTTGTTGTTCTGGCAATTTAAAAAGAAAGAGGATTTA
AACGTACCTATAGAAACAAAGATTTATGGTTAAAGAATGAGATCAGAAGT
CTAGAATGTTTTTAAATTGTGATATATTTTACAACATCCGTTATTACTTT
GAGACATTTGTCCTAATCTACGTATAAAACTCAATCTAGGGCTAAAGATT
CTTTATACCATCTTAGGTTCATTCATCTTAGGCTATTTGAACCACTTTTT
AATTTAATATGAAAGACACCATGCAGTGTTTTCCGAGACTACATAGATCA
TTTTATCACATACCTACCAAGCCTGTTGGAAATAGGTTTTGATAATTTAA
GTAGGGACCTATACAAAATATATTACATTTATCAGATTTTTAAATACATT
CAATTAAGAATTTAACATCACCTTAAATTTGAATTCAATCTACCGTTATT
TCAAACTCACAAATATAACTGCATTATGAATACTTACATAATGTAGTAAG
ACAAGATGTTTGACAGGTTCGTGTGTAATTTTCTATTAATGTTTTTACAT
TGCCTTGTTTTTATGTAAAATAAAAAATATGGGCAACTGGTTTGTTAACA
ACACAATTTCTTCTTAGCATTTCAAAAATATATATAAAGTTGTTCTTTTT
CCTATTTCATGAACTATGTTTTTTTTTAAAATAACATGGTTAAGTTTTAT
ATATATTTACGTTTGTTTCAGGAATGTCTACTTGTGACTTTTTATCAATT
AAAAATAATATTTGGAAGAAAGAGCTTATTAAGTATAAGCTTGAAGTAAA
ATTAGACCTCTCTTTCCATGTAGATTACTGTTTGTACTGATGGTTTCACC
CTTCAGAAGGCACTGTCATATTAATATTTAAATTTTATAATCGCTGAACT
TATTACACCCAACAATACAGAAAGGCAGTTACACTGAAGAACTTAACTTA
GAATAAAATGGAAGCAAACAGGTTTTCTAAAAACTTTTTTAAGTGACCAG
GTCTCGCTCTGTCACCCAGGCTAGAGTGCAATGGCATGATCATAGCTCTC
TGCAGCCTCAACTCTGGGCTCAAGCAACCCTCCTGCCTCAGCCTCCCAAG
TAGCTAAGACTACAGGTACATGCCACCATGCCTGGCTAATATTTAAATTT
TTGTAGATAAGGGGTCTTGCTATGTTGCCCAGGCTAGTCTCAAACTCCTG
GCTTCAAGTGTTCCTACTGTCATGACCTGCCAACATGCTGGGGTTACAGG
CATGAGCCACCATGCCCCAAACAGGTTTGAACACAAATCTTTCGGATGAA
AATTAGAGAACCTAATTTTAGCTTTTTGATAGTTACCTAGTTTGCAAAAG
ATTTGGGTGACTTGTGAGCTGTTTTTAAATGCTGATTGTTGAACATCACA
ACCCAAAATACTTAGCATGATTTTATAGAGTTTTGATAGCTTTATTAAAA
AGAGTGAAAATAAAATGCATATGTAAATAAAGCAGTTCTAAATAGCTATT
TCAGAGAAATGTTAATAGAAGTGCTGAAAGAAGGGCCAACTAAATTAGGA
TGGCCAGGGAATTGGCCTGGGTTTAGGACCTATGTATGAAGGCCACCAAT
TTTTTAAAAATATCTGTGGTTTATTATGTTATTATCTTCTTGAGGAAAAC
AATCAAGAATTGCTTCATGAAAATAAATAAATAGCCATGAATATCATAAA
GCTGTTTACATAGGATTCTTTACAAATTTCATAGATCTATGAATGCTCAA
AATGTTTGAGTTTGCCATAAATTATATTGTAGTTATATTGTAGTTATACT
TGAGACTGACACATTGTAATATAATCTAAGAATAAAAGTTATACAAAATA
A
SCN10A (Uniprot #: Q9Y5Y9)
Synonyms: NaV1.8, PN3, hPN3
(SEQ ID NO: 2436)
MEFPIGSLETNNFRRFTPESLVEIEKQTAAKQGTKKAREKHREQKDQEEKPRPQLDLKACNQLPKFYGELPA
ELIGEPLEDLDPFYSTHRTFMVLNKGRTTSRFSATRALWLESPENLIRRTAIKVSVHSWFSLFITVTILVNCVC
MTRTDLPEKIEYVFTVIYTFEALIKILARGFCLNEFTYLRDPWNWLDFSVITLAYVGTAIDLRGISGLRTFRV
LRALKTVSVIPGLKVIVGALIHSVKKLADVTILTIFCLSVFALVGLQLFKGNLKNKCVKNDMAVNETTNYSS
HRKPDIYINKRGTSDPLLCGNGSDSGHCPDGYICLKTSDNPDFNYTSFDSFAWAFLSLFRLMTQDSWERLY
QQTLRTSGKIYMIFFVLVIFLGSFYLVNLILAVVTMAYEEQNQATTDEIEAKEKKFQEALEMLRKEQEVLAA
LGIDTTSLHSHNGSPLTSKNASERRHRIKPRVSEGSTEDNKSPRSDPYNQRRMSFLGLASGKRRASHGSVFH
FRSPGRDISLPEGVTDDGVFPGDHESHRGSLLLGGGAGQQGPLPRSPLPQPSNPDSRHGEDEHQPPPTSELAP
GAVDVSAFDAGQKKTFLSAEYLDEPFRAQRAMSVVSIITSVLEELEESEQKCPPCLTSLSQKYLIWDCCPM
WVKLKTILFGLVTDPFAELTITLCIVVNTIFMAMEHHGMSPTFEAMLQIGNIVFTIFFTAEMVFKIIAFDPYYY
FQKKWNIFDCIIVTVSLLELGVAKKGSLSVLRSFRLLRVFKLAKSWPTLNTLIKIIGNSVGALGNLTIILAIIVF
VFALVGKQLLGENYRNNRKNISAPHEDWPRWHMHDFFHSFLIVFRILCGEWIENMWACMEVGQKSICLILF
LTVMVLGNLVVLNLFIALLLNSFSADNLTAPEDDGEVNNLQVALARIQVFGHRTKQALCSFFSRSCPFPQPK
AEPELVVKLPLSSSKAENHIAANTARGSSGGLQAPRGPRDEHSDFIANPTVWVSVPIAEGESDLDDLEDDGG
EDAQSFQQEVIPKGQQEQLQQVERCGDHLTPRSPGTGTSSEDLAPSLGETWKDESVPQVPAEGVDDTSSSE
GSTVDCLDPEEILRKIPELADDLEEPDDCFTEGCIRHCPCCKLDTTKSPWDVGWQVRKTCYRIVEHSWFESF
IIFMILLSSGSLAFEDYYLDQKPTVKALLEYTDRVFTFIFVFEMLLKWVAYGFKKYFTNAWCWLDFLIVNIS
LISLTAKILEYSEVAPIKALRTLRALRPLRALSRFEGMRVVVDALVGAIPSIMNVLLVCLIFWLIFSIMGVNLF
AGKFWRCINYTDGEFSLVPLSIVNNKSDCKIQNSTGSFFWVNVKVNFDNVAMGYLALLQVATFKGWMDI
MYAAVDSREVNMQPKWEDNVYMYLYFVIFIIFGGFFTLNLFVGVIIDNFNQQKKKLGGQDIFMTEEQKKY
YNAMKKLGSKKPQKPIPRPLNKFQGFVFDIVTRQAFDITIMVLICLNMITMMVETDDQSEEKTKILGKINQF
FVAVFTGECVMKMFALRQYYFINGWNVFDFIVVVLSIASLIFSAILKSLQSYFSPTLFRVIRLARIGRILRLIR
AAKGIRTLLFALMMSLPALFNIGLLLFLVMFIYSIFGMSSFPHVRWEAGIDDMFNFQTFANSMLCLFQITTSA
GWDGLLSPILNTGPPYCDPNLPNSNGTRGDCGSPAVGIIFFTTYHISFLIMVNMYIAVILENENVATEESTEPL
SEDDFDMFYETWEKFDPEATQFITFSALSDFADTLSGPLRIPKPNRNILIQMDLPLVPGDKIHCLDILFAFTKN
VLGESGELDSLKANMEEKFMATNLSKSSYEPIATTLRWKQEDISATVIQKAYRSYVLHRSMALSNTPCVPR
AEEEAASLPDEGFVAFTANENCVLPDKSETASATSFPPSYESVTRGLSDRVNMRTSSSIQNEDEATSMELIAP
GP
SCN11A (Uniprot #: Q9UI33)
Synonyms: NaV1.9, PN5, SCN12A, SNS2, hNaN
(SEQ ID NO: 2437)
MDDRCYPVIFPDERNFRPFTSDSLAAIEKRIAIQKEKKKSKDQTGEVPQPRPQLDLKASRKLPKLYGDIPREL
IGKPLEDLDPFYRNHKTFMVLNRKRTIYRFSAKHALFIFGPENSIRSLAIRVSVHSLFSMFIIGTVIINCVFMAT
GPAKNSNSNNTDIAECVFTGIYIFEALIKILARGFILDEFSFLRDPWNWLDSIVIGIAIVSYIPGITIKLLPLRTFR
VFRALKAISVVSRLKVIVGALLRSVKKLVNVIILTFFCLSIFALVGQQLFMGSLNLKCISRDCKNISNPEAYD
HCFEKKENSPEFKMCGIWMGNSACSIQYECKHTKINPDYNYTNFDNFGWSFLAMFRLMTQDSWEKLYQQ
TLRTTGLYSVFFFIVVIFLGSFYLINLTLAVVTMAYEEQNKNVAAEIEAKEKMFQEAQQLLKEEKEALVAM
GIDRSSLTSLETSYFTPKKRKLFGNKKRKSFFLRESGKDQPPGSDSDEDCQKKPQLLEQTKRLSQNLSLDHF
DEHGDPLQRQRALSAVSILTITMKEQEKSQEPCLPCGENLASKYLVWNCCPQWLCVKKVLRTVMTDPFTE
LAITICHIINTVFLAMEHHKMEASFEKMLNIGNLVFTSIFIAEMCLKIIALDPYHYFRRGWNIFDSIVALLSFAD
VMNCVLQKRSWPFLRSFRVLRVFKLAKSWPTLNTLIKIIGNSVGALGSLTVVLVIVIFIFSVVGMQLFGRSEN
SQKSPKLCNPTGPTVSCLRHWHMGDFWHSFLVVFRILCGEWIENMWECMQEANASSSLCVIVFILITVIGKL
VVLNLFIALLLNSFSNEERNGNLEGEARKTKVQLALDRFRRAFCFVRHTLEHFCHKWCRKQNLPQQKEVA
GGCAAQSKDIIPLVMEMKRGSETQEELGILTSVPKTLGVRHDWTWLAPLAEEEDDVEFSGEDNAQRITQPE
PEQQAYELHQENKKPTSQRVQSVEIDMFSEDEPHLTIQDPRKKSDVTSILSECSTIDLQDGFGWLPEMVPKK
QPERCLPKGFGCCFPCCSVDKRKPPWVIWWNLRKTCYQIVKHSWFESFIIFVILLSSGALIFEDVHLENQPKI
QELLNCTDIIFTHIFILEMVLKWVAFGFGKYFTSAWCCLDFIIVIVSVTTLINLMELKSFRTLRALRPLRALSQ
FEGMKVVVNALIGAIPAILNVLLVCLIFWLVFCILGVYFFSGKFGKCINGTDSVINYTIITNKSQCESGNESWI
NQKVNFDNVGNAYLALLQVATFKGWMDIIYAAVDSTEKEQQPEFESNSLGYIYFVVFUIFGSFFTLNLFIGVI
IDNENQQQKKLGGQDIFMTEEQKKYYNAMKKLGSKKPQKPIPRPLNKCQGLVEDIVTSQIFDIHUISLIILNM
ISMMAESYNQPKAMKSILDHLNWVFVVIFTLECLIKIFALRQYYFINGWNLFDCVVVLLSIVSTMISTLENQ
EHIPFPPTLFRIVRLARIGRILRLVRAARGIRTLLFALMMSLPSLFNIGLLLFLIMFIYAILGMNWFSKVNPESGI
DDIFNFKTFASSMLCLFQISTSAGWDSLLSPMLRSKESCNSSSENCHLPGIATSYFVSYHUSFLIVVNMYIAVIL
ENENTATEESEDPLGEDDFDIFYEVWEKFDPEATQFIKYSALSDFADALPEPLRVAKPNKYQFLVMDLPMV
SEDRLHCMDILFAFTARVLGGSDGLDSMKAMMEEKFMEANPLKKLYEPIVTTTKRKEEERGAAIIQKAFRK
YMMKVTKGDQGDQNDLENGPHSPLQTLCNGDLSSFGVAKGKVHCD
SCN3A (Uniprot #: Q9NY46)
Synonyms: NaV1.3, KIAA1356, NAC3
(SEQ ID NO: 2438)
MAQALLVPPGPESFRLFTRESLAAIEKRAAEEKAKKPKKEQDNDDENKPKPNSDLEAGKNLPFIYGDIPPEM
VSEPLEDLDPYYINKKTFIVMNKGKAIFRFSATSALYILTPLNPVRKIAIKILVHSLFSMLIMCTILTNCVFMTL
SNPPDWTKNVEYTFTGIYTFESLIKILARGFCLEDFTFLRDPWNWLDFSVIVMAYVTEFVSLGNVSALRTFR
VLRALKTISVIPGLKTIVGALIQSVKKLSDVMILTVFCLSVFALIGLQLFMGNLRNKCLQWPPSDSAFETNTT
SYFNGTMDSNGTFVNVTMSTFNWKDYIGDDSHFYVLDGQKDPLLCGNGSDAGQCPEGYICVKAGRNPNY
GYTSFDTFSWAFLSLFRLMTQDYWENLYQLTLRAAGKTYMIFFVLVIFLGSFYLVNLILAVVAMAYEEQN
QATLEEAEQKEAEFQQMLEQLKKQQEEAQAVAAASAASRDFSGIGGLGELLESSSEASKLSSKSAKEWRN
RRKKRRQREHLEGNNKGERDSFPKSESEDSVKRSSFLFSMDGNRLTSDKKFCSPHQSLLSIRGSLFSPRRNS
KTSIFSFRGRAKDVGSENDFADDEHSTFEDSESRRDSLFVPHRHGERRNSNVSQASMSSRMVPGLPANGKM
HSTVDCNGVVSLVGGPSALTSPTGQLPPEGTTTETEVRKRRLSSYQISMEMLEDSSGRQRAVSIASILTNTM
EELEESRQKCPPCWYRFANVFLIWDCCDAWLKVKHLVNLIVMDPFVDLAITICIVLNTLFMAMEHYPMTE
QFSSVLTVGNLVFTGIFTAEMVLKIIAMDPYYYFQEGWNIFDGIIVSLSLMELGLSNVEGLSVLRSFRLLRVF
KLAKSWPTLNMLIKIIGNSVGALGNLTLVLAIIVFIFAVVGMQLFGKSYKECVCKINDDCTLPRWHMNDFF
HSFLIVFRVLCGEWIETMWDCMEVAGQTMCLIVEMLVMVIGNLVVLNLFLALLLSSFSSDNLAATDDDNE
MNNLQIAVGRMQKGIDYVKNKMRECFQKAFFRKPKVIEIHEGNKIDSCMSNNTGIEISKELNYLRDGNGTT
SGVGTGSSVEKYVIDENDYMSFINNPSLTVTVPIAVGESDFENLNTEEFSSESELEESKEKLNATSSSEGSTV
DVVLPREGEQAETEPEEDLKPEACFTEGCIKKFPFCQVSTEEGKGKIWWNLRKTCYSIVEHNWFETFIVFMI
LLSSGALAFEDIYIEQRKTIKTMLEYADKVFTYIFILEMLLKWVAYGFQTYFTNAWCWLDFLIVDVSLVSLV
ANALGYSELGAIKSLRTLRALRPLRALSRFEGMRVVVNALVGAIPSIMNVLLVCLIFWLIFSIMGVNLFAGK
FYHCVNMTTGNMFDISDVNNLSDCQALGKQARWKNVKVNFDNVGAGYLALLQVATFKGWMDIMYAAV
DSRDVKLQPVYEENLYMYLYFVIFIIFGSFFTLNLFIGVIIDNFNQQKKKFGGQDIFMTEEQKKYYNAMKKL
GSKKPQKPIPRPANKFQGMVFDFVTRQVFDISIMILICLNMVTMMVETDDQGKYMTLVLSRINLVFIVLFTG
EFVLKLVSLRHYYFTIGWNIFDFVVVILSIVGMFLAEMIEKYFVSPTLFRVIRLARIGRILRLIKGAKGIRTLLF
ALMMSLPALFNIGLLLFLVMFIYAIFGMSNFAYVKKEAGIDDMENFETFGNSMICLFQITTSAGWDGLLAPI
LNSAPPDCDPDTIHPGSSVKGDCGNPSVGIFFFVSYIIISELVVVNMYIAVILENFSVATEESAEPLSEDDFEMF
YEVWEKFDPDATQFIEFSKLSDFAAALDPPLLIAKPNKVQLIAMDLPMVSGDRIHCLDILFAFTKRVLGESG
EMDALRIQMEDRFMASNPSKVSYEPITTTLKRKQEEVSAAIIQRNFRCYLLKQRLKNISSNYNKEAIKGRIDL
PIKQDMIIDKLNGNSTPEKTDGSSSTTSPPSYDSVTKPDKEKFEKDKPEKESKGKEVRENQK
CACNAIH (Uniprot #: 095180)
Synonyms: CaV3.2, CACIH
(SEQ ID NO: 2439)
MTEGARAADEVRVPLGAPPPGPAALVGASPESPGAPGREAERGSELGVSPSESPAAERGAELGADEEQRVP
YPALAATVFFCLGQTTRPRSWCLRLVCNPWFEHVSMLVIMLNCVTLGMFRPCEDVECGSERCNILEAFDAF
IFAFFAVEMVIKMVALGLFGQKCYLGDTWNRLDFFIVVAGMMEYSLDGHNVSLSAIRTVRVLRPLRAINR
VPSMRILVTLLLDTLPMLGNVLLLCFFVFFIFGIVGVQLWAGLLRNRCFLDSAFVRNNNLTFLRPYYQTEEG
EENPFICSSRRDNGMQKCSHIPGRRELRMPCTLGWEAYTQPQAEGVGAARNACINWNQYYNVCRSGDSNP
HNGAINFDNIGYAWIAIFQVITLEGWVDIMYYVMDAHSFYNFIYFILLIIVGSFFMINLCLVVIATQFSETKQR
ESQLMREQRARHLSNDSTLASFSEPGSCYEELLKYVGHIFRKVKRRSLRLYARWQSRWRKKVDPSAVQGQ
GPGHRQRRAGRHTASVHHLVYHHHHHHHHHYHFSHGSPRRPGPEPGACDTRLVRAGAPPSPPSPGRGPPD
AESVHSIYHADCHIEGPQERARVAHAAATAAASLRLATGLGTMNYPTILPSGVGSGKGSTSPGPKGKWAG
GPPGTGGHGPLSLNSPDPYEKIPHVVGEHGLGQAPGHLSGLSVPCPLPSPPAGTLTCELKSCPYCTRALEDPE
GELSGSESGDSDGRGVYEFTQDVRHGDRWDPTRPPRATDTPGPGPGSPQRRAQQRAAPGEPGWMGRLWV
TFSGKLRRIVDSKYFSRGIMMAILVNTLSMGVEYHEQPEELTNALEISNIVFTSMFALEMLLKLLACGPLGYI
RNPYNIFDGIIVVISVWEIVGQADGGLSVLRTFRLLRVLKLVRFLPALRRQLVVLVKTMDNVATFCTLLMLF
IFIFSILGMHLFGCKFSLKTDTGDTVPDRKNFDSLLWAIVTVFQILTQEDWNVVLYNGMASTSSWAALYFV
ALMTFGNYVLENLLVAILVEGFQAEGDANRSDTDEDKTSVHFEEDFHKLRELQTTELKMCSLAVTPNGHL
EGRGSLSPPLIMCTAATPMPTPKSSPFLDAAPSLPDSRRGSSSSGDPPLGDQKPPASLRSSPCAPWGPSGAWS
SRRSSWSSLGRAPSLKRRGQCGERESLLSGEGKGSTDDEAEDGRAAPGPRATPLRRAESLDPRPLRPAALPP
TKCRDRDGQVVALPSDFFLRIDSHREDAAELDDDSEDSCCLRLHKVLEPYKPQWCRSREAWALYLFSPQN
RFRVSCQKVITHKMFDHVVLVFIFLNCVTIALERPDIDPGSTERVFLSVSNYIFTAIFVAEMMVKVVALGLLS
GEHAYLQSSWNLLDGLLVLVSLVDIVVAMASAGGAKILGVLRVLRLLRTLRPLRVISRAPGLKLVVETLISS
LRPIGNIVLICCAFFIIFGILGVQLFKGKFYYCEGPDTRNISTKAQCRAAHYRWVRRKYNFDNLGQALMSLF
VLSSKDGWVNIMYDGLDAVGVDQQPVQNHNPWMLLYFISFLLIVSFFVLNMFVGVVVENFHKCRQHQEA
EEARRREEKRLRRLERRRRSTFPSPEAQRRPYYADYSPTRRSIHSLCTSHYLDLFITFIICVNVITMSMEHYNQ
PKSLDEALKYCNYVFTIVFVFEAALKLVAFGFRRFFKDRWNQLDLAIVLLSLMGITLEEIEMSAALPINPTIIR
IMRVLRIARVLKLLKMATGMRALLDTVVQALPQVGNLGLLFMLLFFIYAALGVELFGRLECSEDNPCEGLS
RHATFSNFGMAFLTLFRVSTGDNWNGIMKDTLRECSREDKHCLSYLPALSPVYFVTFVLVAQFVLVNVVV
AVLMKHLEESNKEAREDAELDAEIELEMAQGPGSARRVDADRPPLPQESPGARDAPNLVARKVSVSRMLS
LPNDSYMFRPVVPASAPHPRPLQEVEMETYGAGTPLGSVASVHSPPAESCASLQIPLAVSSPARSGEPLHAL
SPRGTARSPSLSRLLCRQEAVHTDSLEGKIDSPRDTLDPAEPGEKTPVRPVTQGGSLQSPPRSPRPASVRTRK
HTFGQRCVSSRPAAPGGEEAEASDPADEEVSHITSSACPWQPTAEPHGPEASPVAGGERDLRRLYSVDAQG
FLDKPGRADEQWRPSAELGSGEPGEAKAWGPEAEPALGARRKKKMSPPCISVEPPAEDEGSARPSAAEGGS
TTLRRRTPSCEATPHRDSLEPTEGSGAGGDPAAKGERWGQASCRAEHLTVPSFAFEPLDLGVPSGDPFLDGS
HSVTPESRASSSGAIVPLEPPESEPPMPVGDPPEKRRGLYLTVPQCPLEKPGSPSATPAPGGGADDPV
HCN1 (Uniprot #: 060741)
Synonyms: BCNG1
(SEQ ID NO: 2440)
MEGGGKPNSSSNSRDDGNSVFPAKASATGAGPAAAEKRLGTPPGGGGAGAKEHGNSVCFKVDGGGGGG
GGGGGGEEPAGGFEDAEGPRRQYGFMQRQFTSMLQPGVNKFSLRMFGSQKAVEKEQERVKTAGFWIIHP
YSDFRFYWDLIMLIMMVGNLVIIPVGITFFTEQTTTPWIIFNVASDTVFLLDLIMNFRTGTVNEDSSEIILDPK
VIKMNYLKSWFVVDFISSIPVDYIFLIVEKGMDSEVYKTARALRIVRFTKILSLLRLLRLSRLIRYIHQWEEIF
HMTYDLASAVVRIFNLIGMMLLLCHWDGCLQFLVPLLQDFPPDCWVSLNEMVNDSWGKQYSYALFKAM
SHMLCIGYGAQAPVSMSDLWITMLSMIVGATCYAMFVGHATALIQSLDSSRRQYQEKYKQVEQYMSFHK
LPADMRQKIHDYYEHRYQGKIFDEENILNELNDPLREEIVNFNCRKLVATMPLFANADPNFVTAMLSKLRF
EVFQPGDYIIREGAVGKKMYFIQHGVAGVITKSSKEMKLTDGSYFGEICLLTKGRRTASVRADTYCRLYSLS
VDNFNEVLEEYPMMRRAFETVAIDRLDRIGKKNSILLQKFQKDLNTGVENNQENEILKQIVKHDREMVQAI
APINYPQMTTLNSTSSTTTPTSRMRTQSPPVYTATSLSHSNLHSPSPSTQTPQPSAILSPCSYTTAVCSPPVQSP
LAARTFHYASPTASQLSLMQQQPQQQVQQSQPPQTQPQQPSPQPQTPGSSTPKNEVHKSTQALHNTNLTRE
VRPLSASQPSLPHEVSTLISRPHPTVGESLASIPQPVTAVPGTGLQAGGRSTVPQRVTLFRQMSSGAIPPNRG
VPPAPPPPAAALPRESSSVLNTDPDAEKPRFASNL
HCN2 (Uniprot #: Q9UL51)
Synonyms:BCNG2
(SEQ ID NO: 2441)
MDARGGGGRPGESPGATPAPGPPPPPPPAPPQQQPPPPPPPAPPPGPGPAPPQHPPRAEALPPEAADEGGPRG
RLRSRDSSCGRPGTPGAASTAKGSPNGECGRGEPQCSPAGPEGPARGPKVSFSCRGAASGPAPGPGPAEEA
GSEEAGPAGEPRGSQASFMQRQFGALLQPGVNKFSLRMFGSQKAVEREQERVKSAGAWIIHPYSDFRFYW
DFTMLLFMVGNLIIIPVGITFFKDETTAPWIVENVVSDTFFLMDLVLNFRTGIVIEDNTEIILDPEKIKKKYLRT
WFVVDFVSSIPVDYIFLIVEKGIDSEVYKTARALRIVRFTKILSLLRLLRLSRLIRYIHQWEEIFHMTYDLASA
VMRICNLISMMLLLCHWDGCLQFLVPMLQDFPRNCWVSINGMVNHSWSELYSFALFKAMSHMLCIGYGR
QAPESMTDIWLTMLSMIVGATCYAMFIGHATALIQSLDSSRRQYQEKYKQVEQYMSFHKLPADFRQKIHD
YYEHRYQGKMFDEDSILGELNGPLREEIVNFNCRKLVASMPLFANADPNFVTAMLTKLKFEVFQPGDYIIR
EGTIGKKMYFIQHGVVSVLTKGNKEMKLSDGSYFGEICLLTRGRRTASVRADTYCRLYSLSVDNFNEVLEE
YPMMRRAFETVAIDRLDRIGKKNSILLHKVQHDLNSGVFNNQENAIIQEIVKYDREMVQQAELGQRVGLFP
PPPPPPQVTSAIATLQQAAAMSFCPQVARPLVGPLALGSPRLVRRPPPGPAPAAASPGPPPPASPPGAPASPR
APRTSPYGGLPAAPLAGPALPARRLSRASRPLSASQPSLPHGAPGPAASTRPASSSTPRLGPTPAARAAAPSP
DRRDSASPGAAGGLDPQDSARSRLSSNL
ANO1 (Uniprot #: Q5XXA6)
Synonyms: DOG1, ORAOV2, TAOS2, TMEM16A
(SEQ ID NO: 2442)
MRVNEKYSTLPAEDRSVHIINICAIEDIGYLPSEGTLLNSLSVDPDAECKYGLYFRDGRRKVDYILVYHHKR
PSGNRTLVRRVQHSDTPSGARSVKQDHPLPGKGASLDAGSGEPPMDYHEDDKRFRREEYEGNLLEAGLEL
ERDEDTKIHGVGFVKIHAPWNVLCREAEFLKLKMPTKKMYHINETRGLLKKINSVLQKITDPIQPKVAEHR
PQTMKRLSYPFSREKQHLEDLSDKDSFFDSKTRSTIVYEILKRTTCTKAKYSMGITSLLANGVYAAAYPLHD
GDYNGENVEFNDRKLLYEEWARYGVFYKYQPIDLVRKYFGEKIGLYFAWLGVYTQMLIPASIVGIVFLYG
CATMDENIPSMEMCDQRHNITMCPLCDKTCSYWKMSSACATARASHLFDNPATVFFSVFMALWAATFME
HWKRKQMRLNYRWDLTGFEEEEEAVKDHPRAEYEARVLEKSLKKESRNKEKRRHIPEESTNKWKQRVKT
AMAGVKLTDKVKLTWRDRFPAYLINLVSIIFMIAVTFAIVLGVIIYRISMAAALAMNSSPSVRSNIRVTVTA
TAVIINLVVIILLDEVYGCIARWLTKIEVPKTEKSFEERLIFKAFLLKFVNSYTPIFYVAFFKGRFVGRPGDYV
YIFRSFRMEECAPGGCLMELCIQLSIIMLGKQLIQNNLFEIGIPKMKKLIRYLKLKQQSPPDHEECVKRKQRY
EVDYNLEPFAGLTPEYMEMIIQFGFVTLFVASFPLAPLFALLNNIIEIRLDAKKFVTELRRPVAVRAKDIGIW
YNILRGIGKLAVIINAFVISFTSDFIPRLVYLYMYSKNGTMHGFVNHTLSSENVSDFQNGTAPNDPLDLGYEV
QICRYKDYREPPWSENKYDISKDFWAVLAARLAFVIVFQNLVMFMSDFVDWVIPDIPKDISQQIHKEKVLM
VELFMREEQDKQQLLETWMEKERQKDEPPCNHHNTKACPDSLGSPAPSHAYHGGVL

### Example 3: C to T Base Editing to Introduce a Premature Stop Codon in Mouse Neuro-2a Cells

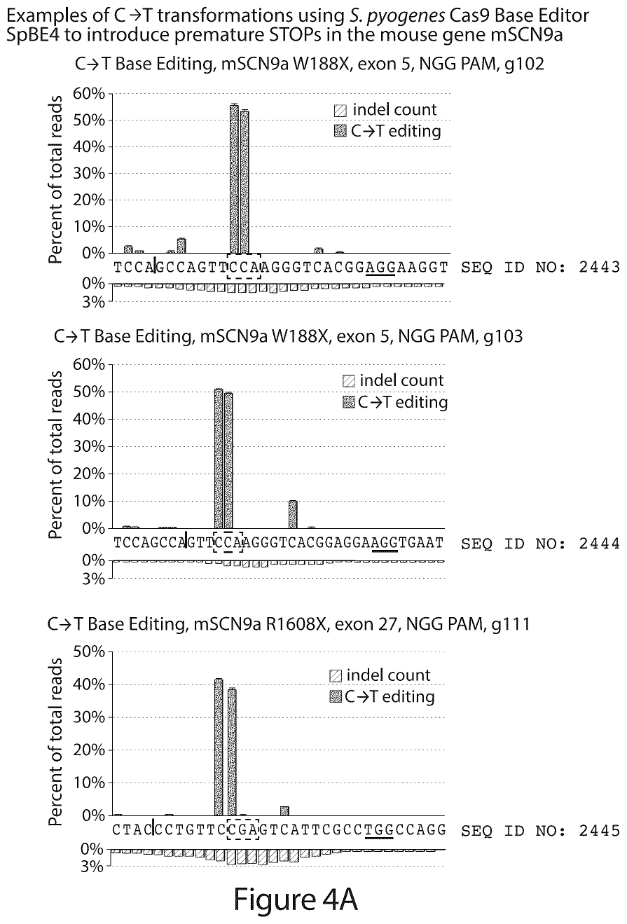

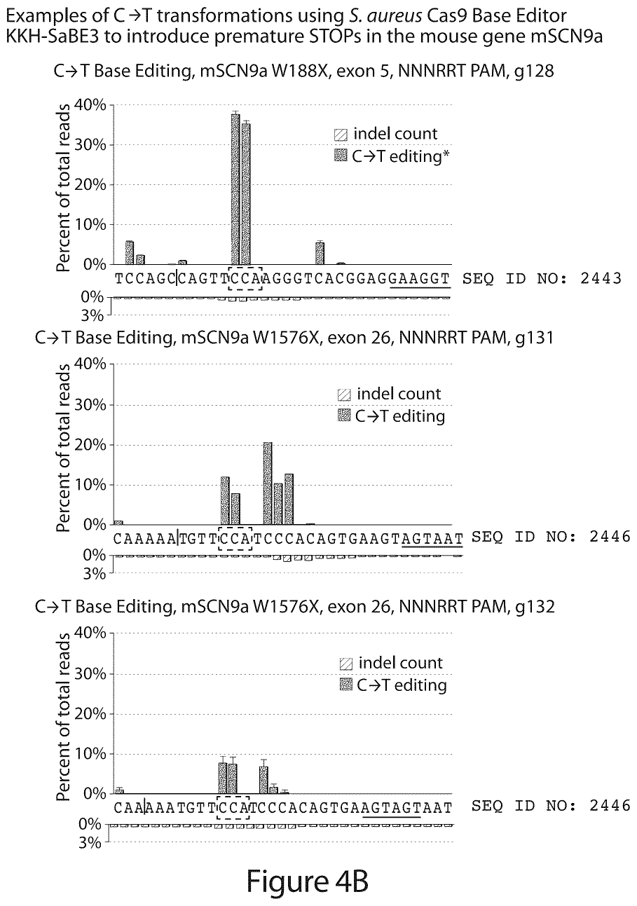

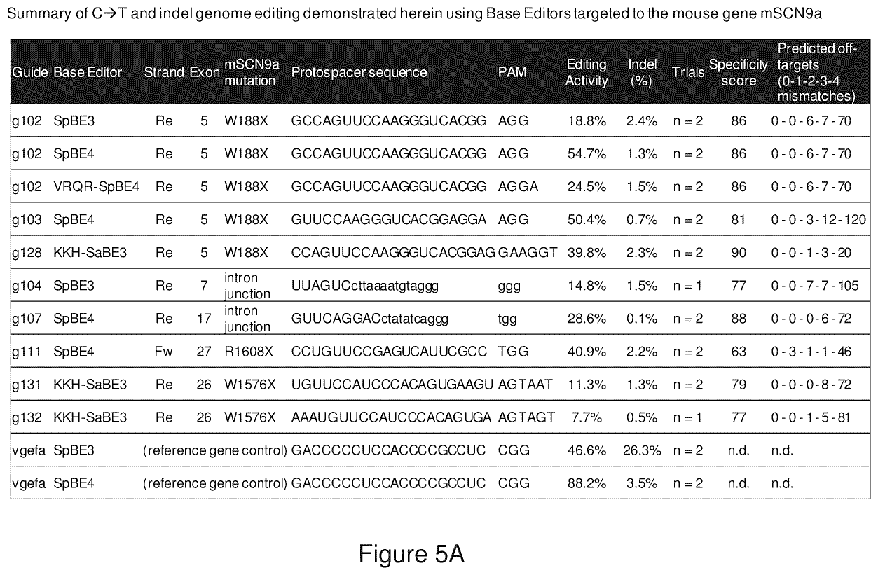

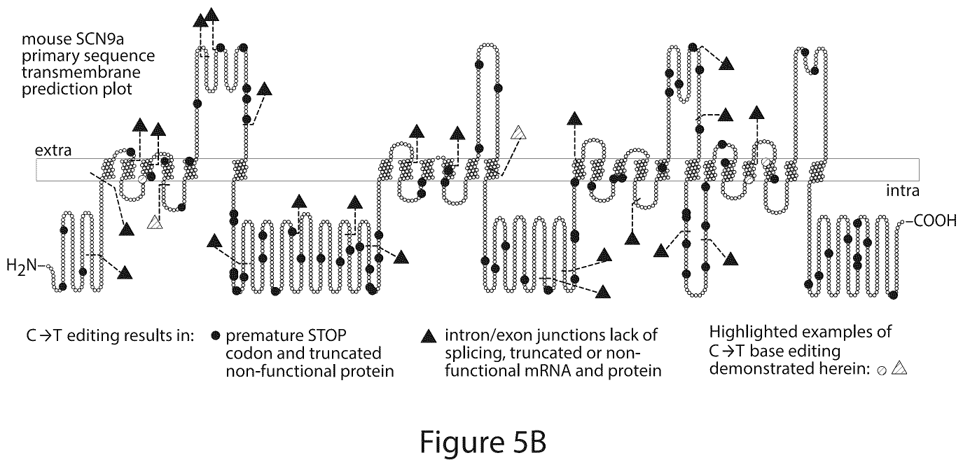

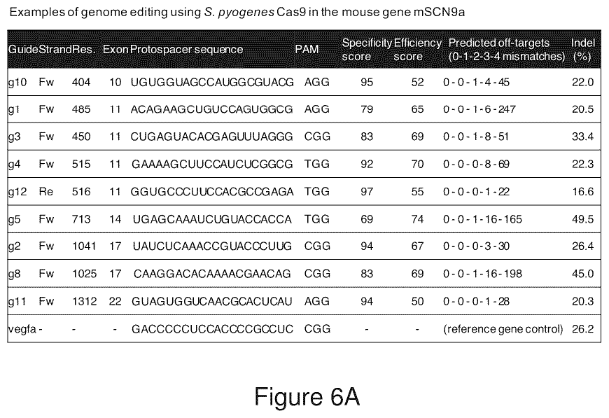

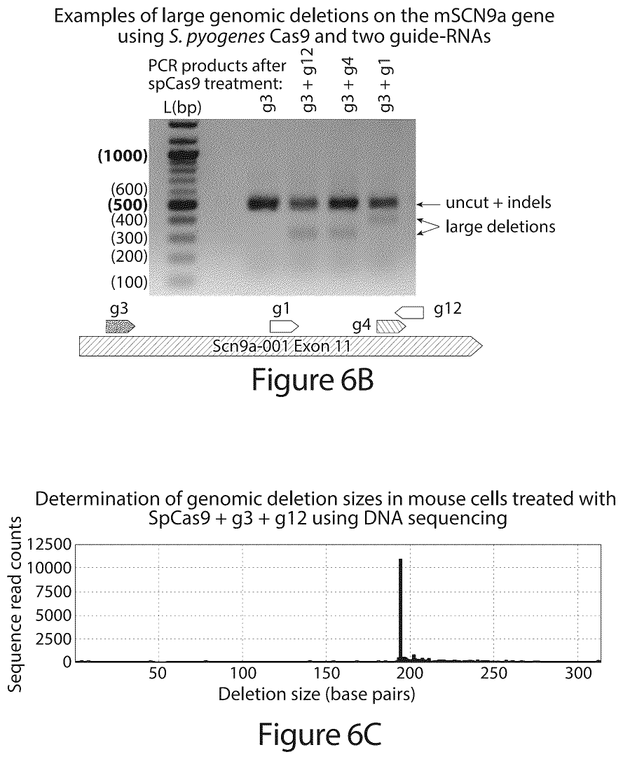

On day 1, a culture of mouse Neuro-2a cells (ATCC) was resuspended using trypsin (TrypLE), and diluted to 1.25×10⁵ cells/mL with DMEM supplemented with 10% FBS and no antibiotics. This suspension of cells (250 μL) was used to seed a 48-well plate coated with poly-D-Lysine, and incubated at 37° C. with 5% CO₂ for 24 hours. On day 2, each well was treated with a cationic lipid-DNA complex comprising 1.5 μL Lipofectamine 3000 and 1 μL of P3000 Reagent (ThermoFisher Scientific), 750 ng of base editor and 250 ng of sgRNA expression plasmids prepared as per the manufacturer's recommendation in a total volume of 25 μL DMEM. The base editing expression vector used the base editor 4 (BE4) architecture as described in Komor et al. 2017.⁵¹ The sgRNA expression plasmid contained the protospacer RNA sequence and the S. pyogenes guide-RNA scaffold driven by a U6 promoter. The transfected Neuro-2a cells were incubated for 72 hours in the same media. On day 5, the cells were resuspended with trypsin (TrypLE), centrifuged, and the cell pellets were washed three times with PBS. The cells were treated with 75 μL of lysis buffer (comprising: 10 mM Tris-HCl pH 8, 0.05% sodium dodecyl sulfate, 25 ug/mL Proteinase K) and incubated at 37° C. for 1 hour in a thermocycler, followed by 80° C. for 20 minutes. The lysate was diluted 1:25 in water, and the target genomic loci were PCR amplified for high-throughput DNA sequencing as described in Komor et al. 2016.⁵² See FIGS. 4-6 for non-limiting examples of the results obtained from C→T base editing treatments using guide-RNAs targeted to the NaV1.7/SCN9A gene in the mouse Neuro-2a cell line.

### REFERENCES

1 Waxman, S. G. & Zamponi, G. W. Regulating excitability of peripheral afferents: emerging ion channel targets. Nature neuroscience 17, 153-163, (2014).
2 Guedon. J. M. et al. Current gene therapy using viral vectors for chronic pain. Molecular pain 11, 27, (2015).
3 Kumar. S., Ruchi, R., James, S. R. & Chidiac, E. J. Gene therapy for chronic neuropathic pain: how does it work and where do we stand today? Pain Med 12, 808-822, (2011).
4 Sapunar, D., Kostic, S., Banozic, A. & Puljak, L. Dorsal root ganglion—a potential new therapeutic target for neuropathic pain. Journal of pain research 5, 31-38, (2012).
5 Komor, A. C., Kim, Y. B., Packer, M. S., Zuris, J. A. & Liu, D. R. Programmable editing of a target base in genomic DNA without double-stranded DNA cleavage. Nature 533, 420-424, (2016).
6 Cong, L. et al. Multiplex genome engineering using CRISPR/Cas systems. Science 339, 819-823, (2013).
7 Jinek. M. et al. A programmable dual-RNA-guided DNA endonuclease in adaptive bacterial immunity. Science 337, 816-821, (2012).
8 Mali, P. et al. RNA-guided human genome engineering via Cas9. Science 339, 823-826, (2013).
9 Ran, F. A. et al. Double nicking by RNA-guided CRISPR Cas9 for enhanced genome editing specificity. Cell 154, 1380-1389, (2013).
10 Guilinger, J. P., Thompson, D. B. & Liu, D. R. Fusion of catalytically inactive Cas9 to FokI nuclease improves the specificity of genome modification. Nature biotechnology 32, 577-582, (2014).
11 Tsai, S. Q. et al. Dimeric CRISPR RNA-guided FokI nucleases for highly specific genome editing. Nature Biotechnology 32, 569-576. (2014).
12 Zetsche, B. et al. Cpf1 is a single RNA-guided endonuclease of a class 2 CRISPR-Cas system. Cell 163, 759-771, (2015).
13 Gao, F., Shen, X. Z., Jiang, F., Wu, Y. & Han, C. DNA-guided genome editing using the Natronobacterium gregoryi Argonaute. Nature Biotechnology 34, 768-773, (2016).
14 Cradick, T. J., Fine, E. J., Antico, C. J. & Bao, G. CRISPR/Cas9 systems targeting β-globin and CCR5 genes have substantial off-target activity. Nucleic acids research, (2013).
15 Holt, N. et al. Human hematopoietic stem/progenitor cells modified by zinc-finger nucleases targeted to CCR5 control HIV-1 in vivo. Nature Biotechnology 28, 839-847, (2010).
16 Shen, Y. & Nemunaitis, J. Herpes simplex virus 1 (HSV-1) for cancer treatment. Cancer gene therapy 13, 975-992, (2006).
17 Smith, G. Herpesvirus transport to the nervous system and back again. Annual review of microbiology 66, 153-176, (2012).
18 Burton, E. A., Fink, D. J. & Glorioso, J. C. Gene delivery using herpes simplex virus vectors. DNA and cell biology 21, 915-936, (2002).
19 Kay, M. A., Glorioso, J. C. & Naldini, L. Viral vectors for gene therapy: the art of turning infectious agents into vehicles of therapeutics. Nature medicine 7, 33-40, (2001).
20 Epstein, A. L. HSV-1-based amplicon vectors: design and applications. Gene therapy 12 Suppl 1, S154-158, (2005).
21 Steiner. I., Kennedy, P. G. & Pachner, A. R. The neurotropic herpes viruses: herpes simplex and varicella-zoster. The Lancet. Neurology 6, 1015-1028, (2007).
22 Lancaster. K. Z. & Pfeiffer, J. K. Limited trafficking of a neurotropic virus through inefficient retrograde axonal transport and the type I interferon response. PLoS pathogens 6, e1000791, (2010).
23 Hotta, H. [Neurotropic viruses—classification, structure and characteristics]. Nihon rinsho. Japanese journal of clinical medicine 55, 777-782, (1997).
24 Kleinstiver, B. P. et al. High-fidelity CRISPR-Cas9 nucleases with no detectable genome-wide off-target effects. Nature 529, 490-495, (2016).
25 Slaymaker. I. M. et al. Rationally engineered Cas9 nucleases with improved specificity. Science 351, 84-88, (2016).
26 Pattanayak, V. et al. High-throughput profiling of off-target DNA cleavage reveals RNA-programmed Cas9 nuclease specificity. Nature biotechnology 31, 839-843. (2013).
27 Wu, X. et al. Genome-wide binding of the CRISPR endonuclease Cas9 in mammalian cells. Nature Biotechnology 32, 670-676, (2014).
28 Hsu, P. D. et al. DNA targeting specificity of RNA-guided Cas9 nucleases. Nature biotechnology 31, 827-832, (2013).
29 Davis, K. M., Pattanayak, V., Thompson, D. B., Zuris, J. A. & Liu, D. R. Small Molecule-Triggered Cas9 Protein With Improved Genome-Editing Specificity. Nature Chemical Biology in press, (2015).
30 Lee, J. H. et al. A monoclonal antibody that targets a NaV1.7 channel voltage sensor for pain and itch relief. Cell 157, 1393-1404, (2014).
31 Bagal, S. K. et al. Recent progress in sodium channel modulators for pain. Bioorganic & medicinal chemistry letters 24, 3690-3699, (2014).
32 King, G. F. & Vetter, I. No gain, no pain: NaV1.7 as an analgesic target. ACS chemical neuroscience 5, 749-751, (2014).
33 Martz, L. Targeting Nav1.7 in pain and itch. Science-Business eXchange 7, (2014, doi:10.1038/scibx.2014.662).
34 Cox, J. J. et al. An SCN9A channelopathy causes congenital inability to experience pain. Nature 444, 894-898, (2006).
35 Cox, J. J. et al. Congenital insensitivity to pain: novel SCN9A missense and in-frame deletion mutations. Human mutation 31, E1670-1686, (2010).
36 Goldberg, Y. P. et al. Loss-of-function mutations in the Nav1.7 gene underlie congenital indifference to pain in multiple human populations. Clinical genetics 71, 311-319, (2007).
37 Yang, Y. et al. Mutations in SCN9A, encoding a sodium channel alpha subunit, in patients with primary erythermalgia. Journal of medical genetics 41, 171-174, (2004).
38 Leipold, E. et al. A de novo gain-of-function mutation in SCN11A causes loss of pain perception. Nature genetics 45, 1399-1404, (2013).
39 Weiss, J. et al. Loss-of-function mutations in sodium channel Nav1.7 cause anosmia. Nature 472, 186-190, (2011).
40 Devigili, G. et al. Paroxysmal itch caused by gain-of-function Nav 1.7 mutation. Pain 155, 1702-1707. (2014).
41 Woods, C. G., Babiker, M. O., Horrocks, I., Tolmie. J. & Kurth, I. The phenotype of congenital insensitivity to pain due to the NaV1.9 variant p.L811P. European journal of human genetics: EJHG 23, 561-563. (2015).
42 Bourinet, E. et al. Silencing of the Cav3.2 T-type calcium channel gene in sensory neurons demonstrates its major role in nociception. The EMBO journal 24, 315-324. (2005).
43 Cho, H. et al. The calcium-activated chloride channel anoctamin 1 acts as a heat sensor in nociceptive neurons. Nature neuroscience 15, 1015-1021. (2012).
44 Andre, S. et al. Axotomy-induced expression of calcium-activated chloride current in subpopulations of mouse dorsal root ganglion neurons. Journal of neurophysiology 90, 3764-3773, (2003).
45 Benarroch, E. E. HCN channels: function and clinical implications. Neurology 80, 304-310. (2013).
46 Emery, E. C., Young, G. T., Berrocoso, E. M., Chen, L. & McNaughton, P. A. HCN2 ion channels play a central role in inflammatory and neuropathic pain. Science 333, 1462-1466, (2011).
47 Shalem. O., Sanjana. N. E. & Zhang, F. High-throughput functional genomics using CRISPR-Cas9. Nature reviews. Genetics 16, 299-311, (2015).
48 Chen, S. et al. Genome-wide CRISPR screen in a mouse model of tumor growth and metastasis. Cell 160, 1246-1260, (2015).
49 Kleinstiver. B. P. et al. Engineered CRISPR-Cas9 nucleases with altered PAM specificities. Nature 523, 481-485, (2015).
50 Doench, J. G. et al. Optimized sgRNA design to maximize activity and minimize off-target effects of CRISPR-Cas9. Nature biotechnology 34, 184-191, (2016).
51 Komor, A. C. et al. Improved base excision repair inhibition and bacteriophage Mu Gam protein yields C:G-toT:A base editors with higher efficiency and product purity. Science Advances 3, eaao4774, (2017).
52 Komor, A. C. et al. Programmable editing of a target base in genomic DNA without double-stranded DNA cleavage. Nature 533, 420-424 (2016).

### EQUIVALENTS AND SCOPE

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the invention encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein.

It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

1-62. (canceled)

63. A composition comprising:
(i) a fusion protein comprising: (a) a guide nucleotide sequence-programmable DNA binding protein domain; and (b) a cytosine deaminase domain; and
(ii) a guide nucleotide sequence targeting the fusion protein of (i) to a target cytosine (C) base in an ion channel-encoding polynucleotide.

64. A composition comprising a neurotropic viral delivery vector comprising a nucleic acid encoding:
(i) a fusion protein comprising: (a) a guide nucleotide sequence-programmable DNA binding protein domain; and (b) a cytosine deaminase domain; and
(ii) a guide nucleotide sequence targeting the fusion protein of (i) to a target cytosine (C) base in an ion channel-encoding polynucleotide.

65. The composition of claim 63, wherein the guide nucleotide sequence comprises the nucleotide sequence of any one of SEQ ID NOs: 339-1456 or 1504-2425.

66. The composition of claim 63, wherein the composition further comprises a pharmaceutically acceptable carrier.

67. (canceled)

68. A method of suppressing pain, the method comprising administering to a subject in need thereof a therapeutically effective amount of the composition of claim 63.

69-79. (canceled)

80. The composition of claim 63, wherein when the fusion protein of (i) targets the cytosine (C) base in an ion channel-encoding polynucleotide, a C to T change occurs in the coding region of the ion channel-encoding polynucleotide.

81. The composition of claim 63, wherein when the fusion protein of (i) targets the cytosine (C) base in an ion channel-encoding polynucleotide, a premature stop codon is introduced in the ion channel-coding sequence that leads to a truncated or non-functional ion channel.

82. The composition of claim 63, wherein when the fusion protein of (i) targets the cytosine (C) base in an ion channel-encoding polynucleotide, a mutation occurs that destabilizes ion-channel protein folding.

83. The composition of claim 63, wherein when the fusion protein of (i) targets the cytosine (C) base in an ion channel-encoding polynucleotide, a C to T change occurs at a C base-paired with the G base in a start codon (AUG).

84. The composition of claim 63, wherein when the fusion protein of (i) targets the cytosine (C) base in an ion channel-encoding polynucleotide, a C to T change occurs in the non-coding region of the ion channel-encoding polynucleotide.

85. The composition of claim 63, wherein the ion channel-encoding polynucleotide is selected from the group consisting of: NaV1.7, NaV1.8, NaV1.9, NaV1.3, CaV3.2, HCN1, HCN2, and Ano1.

86. The composition of claim 63, wherein the ion channel-encoding polynucleotide is NaV1.7 encoded by the SCN9A gene.

87. The composition of claim 63, wherein the guide nucleotide sequence-programmable DNA binding protein domain is selected from the group consisting of: nuclease inactive Cas9 (dCas9) domains, nuclease inactive Cpf1 domains, nuclease inactive Argonaute domains, and variants thereof.

88. The composition of claim 63, wherein the cytosine deaminase domain comprises an apolipoprotein B mRNA-editing complex (APOBEC) family deaminase.

89. The composition of claim 63, wherein the cytosine deaminase domain is selected from the group consisting of APOBEC1, APOBEC2, APOBEC3A, APOBEC3B, APOBEC3C, APOBEC3D, APOBEC3F, APOBEC3G deaminase, APOBEC3H deaminase, APOBEC4 deaminase, activation-induced deaminase (AID), and pmCDA1.

90. The composition of claim 63, wherein the cytosine deaminase domain comprises an amino acid sequence having at least 95% sequence identity to any one of SEQ ID NOs: 271-292, 303, and 2483-2494.

91. The composition of claim 63, wherein the fusion protein further comprises a uracil glycosylase inhibitor (UGI) domain.

92. The composition of claim 91, wherein the UGI domain comprises an amino acid sequence having at least 95% sequence identity to SEQ ID NO: 304.

93. The composition of claim 63, wherein the fusion protein comprises an amino acid sequence having at least 95% sequence identity to any one of SEQ ID NOs: 296-302 and 2495.

94. A method of editing a polynucleotide encoding an ion channel in a dorsal root ganglion (DRG) neuron, the method comprising contacting the ion channel-encoding polynucleotide with the composition of claim 63; wherein the guide nucleic acid molecule is selected from the nucleic acid sequence having at least 95% sequence identity to any one of SEQ ID NOs: 339-1456; and
whereby the contacting results in deamination of the target C base by the fusion protein, resulting in a C to T change in the ion channel-encoding polynucleotide.
